(19) United States
(12) Patent Application Publication
(10) Pub. No.: US 2013/0060013 A1
(43) Pub. Date: Mar. 7, 2013

(54) Compounds useful for treating neurodegenerative disorders

(71) Applicants: Brian Scott Bronk, East Lyme, CT (US); Wesley Francis Austin, Cambridge, MA (US); Steffen Phillip Creaser, Cambridge, MA (US); Nathan Oliver Fuller, Somerville, MA (US); Jed Lee Hubbs, Cambridge, MA (US); Jeffrey Lee Ives, Chester, CT (US); Ruichao Shen, West Roxbury, MA (US)

(72) Inventors: Brian Scott Bronk, East Lyme, CT (US); Wesley Francis Austin, Cambridge, MA (US); Steffen Phillip Creaser, Cambridge, MA (US); Nathan Oliver Fuller, Somerville, MA (US); Jed Lee Hubbs, Cambridge, MA (US); Jeffrey Lee Ives, Chester, CT (US); Ruichao Shen, West Roxbury, MA (US)

(73) Assignee: SATORI PHARMACEUTICALS, INC., Cambridge, MA (US)

(21) Appl. No.: 13/605,956

(22) Filed: Sep. 6, 2012

Related U.S. Application Data
(60) Provisional application No. 61/532,049, filed on Sep. 7, 2011.

(51) Int. Cl. C07J 71/00; C07D 491/052; C07H 17/04

(52) U.S. Cl. 536/6; 540/48; 540/4; 540/576

(57) ABSTRACT

The present invention provides compounds of formula I:

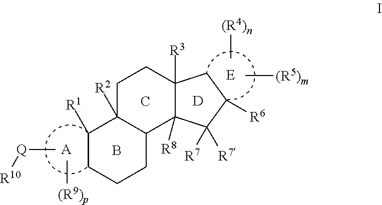

or a pharmaceutically acceptable salt thereof, wherein each of the variables listed above are as defined and described herein, compositions thereof, and methods of using the same.

### CROSS-REFERENCE TO RELATED APPLICATIONS

The present application is a non-provisional application which claims priority to U.S. provisional patent application Ser. No. 61/532,049, filed Sep. 7, 2011, the entirety of each of which is hereby incorporated herein by reference.

### TECHNICAL FIELD OF INVENTION

The present invention relates to pharmaceutically active compounds useful for treating, or lessening the severity of, neurodegenerative disorders.

### BACKGROUND OF THE INVENTION

The central role of the long form of amyloid beta-peptide (also referred to herein as A-beta, A beta, and Aβ), and in particular Aβ(1-42), in Alzheimer's disease has been established through a variety of histopathological, genetic and biochemical studies. See Selkoe, D J, Physiol. Rev. 2001, 81:741-766, Alzheimer's disease: genes, proteins, and therapy, and Younkin S G, J. Physiol. Paris. 1998, 92:289-92, The role of A beta 42 in Alzheimer's disease. Specifically, it has been found that deposition in the brain of Aβ(1-42) is an early and invariant feature of all forms of Alzheimer's disease. In fact, this occurs before a diagnosis of Alzheimer's disease is possible and before the deposition of the shorter primary form of A-beta, Aβ(1-40). See Parvathy S, et al., Arch. Neurol. 2001, 58:2025-32, Correlation between Abetax-40-, Abetax-42-, and Abetax-43-containing amyloid plaques and cognitive decline. Further implication of Aβ(1-42) in disease etiology comes from the observation that mutations in presenilin (gamma secretase) genes associated with early onset familial forms of Alzheimer's disease uniformly result in increased levels of Aβ(1-42). See Ishii K., et al., Neurosci. Lett. 1997, 228:17-20, Increased A beta 42(43)-plaque deposition in early-onset familial Alzheimer's disease brains with the deletion of exon 9 and the missense point mutation (H163R) in the PS-1 gene. Additional mutations in the amyloid precursor protein APP raise total Aβ and in some cases raise Aβ(1-42) alone. See Kosaka T, et al., Neurology, 48:741-5, The beta APP717 Alzheimer mutation increases the percentage of plasma amyloid-beta protein ending at A beta42(43). Although the various APP mutations may influence the type, quantity, and location of Aβ deposited, it has been found that the predominant and initial species deposited in the brain parenchyma is long Aβ (Mann). See Mann D M, et al., Am. J. Pathol. 1996, 148:1257-66, “Predominant deposition of amyloid-beta 42(43) in plaques in cases of Alzheimer's disease and hereditary cerebral hemorrhage associated with mutations in the amyloid precursor protein gene”.

In early deposits of Aβ, when most deposited protein is in the form of amorphous or diffuse plaques, virtually all of the Aβ is of the long form. See Gravina S A, et al., J. Biol. Chem., 270:7013-6, Amyloid beta protein (A beta) in Alzheimer's disease brain. Biochemical and immunocytochemical analysis with antibodies specific for forms ending at A beta 40 or A beta 42(43); Iwatsubo T, et al., Am. J. Pathol. 1996, 149:1823-30, Full-length amyloid-beta (1-42(43)) and amino-terminally modified and truncated amyloid-beta 42(43) deposit in diffuse plaques; and Roher A E, et al., Proc. Natl. Acad. Sci. USA. 1993, 90:10836-40, beta-Amyloid-(1-42) is a major component of cerebrovascular amyloid deposits: implications for the pathology of Alzheimer disease. These initial deposits of Aβ(1-42) then are able to seed the further deposition of both long and short forms of Aβ. See Tamaoka A, et al., Biochem. Biophys. Res. Commun. 1994, 205:834-42, Biochemical evidence for the long-tail form (A beta 1-42/43) of amyloid beta protein as a seed molecule in cerebral deposits of Alzheimer's disease.

In transgenic animals expressing Aβ, deposits were associated with elevated levels of Aβ(1-42), and the pattern of deposition is similar to that seen in human disease with Aβ(1-42) being deposited early followed by deposition of Aβ(1-40). See Rockenstein E, et al., J. Neurosci. Res. 2001, 66:573-82, Early formation of mature amyloid-beta protein deposits in a mutant APP transgenic model depends on levels of Abeta(1-42); and Terai K, et al., Neuroscience 2001, 104:299-310, beta-Amyloid deposits in transgenic mice expressing human beta-amyloid precursor protein have the same characteristics as those in Alzheimer's disease. Similar patterns and timing of deposition are seen in Down's syndrome patients in which Aβ expression is elevated and deposition is accelerated. See Iwatsubo T., et al., Ann. Neurol. 1995, 37:294-9, Amyloid beta protein (A beta) deposition: A beta 42(43) precedes A beta 40 in Down syndrome.

Accordingly, selective lowering of Aβ(1-42) thus emerges as a disease-specific strategy for reducing the amyloid forming potential of all forms of Aβ, slowing or stopping the formation of new deposits of Aβ, inhibiting the formation of soluble toxic oligomers of Aβ, and thereby slowing or halting the progression of neurodegeneration.

### SUMMARY OF THE INVENTION

As described herein, the present invention provides compounds useful for treating or lessening the severity of a neurodegenerative disorder. The present invention also provides methods of treating or lessening the severity of such disorders wherein said method comprises administering to a patient a compound of the present invention, or composition thereof. Said method is useful for treating or lessening the severity of, for example, Alzheimer's disease.

### DETAILED DESCRIPTION OF CERTAIN EMBODIMENTS OF THE INVENTION

### 1. General Description of Compounds of the Invention

According to one embodiment, the present invention provides a compound of formula I:

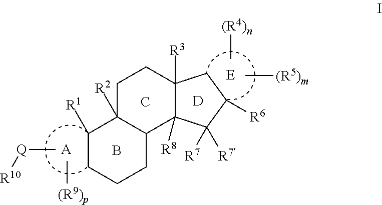

or a pharmaceutically acceptable salt thereof, wherein:
Ring A is a 4-7 membered saturated or partially unsaturated ring having 0-2 heteroatoms independently selected from nitrogen, oxygen, or sulfur;
each of Ring B, Ring C, and Ring D is independently saturated, partially unsaturated or aromatic, or a deuterated derivative thereof.
Ring E is a 4-7 membered saturated, partially unsaturated, or aromatic ring having 0-2 heteroatoms independently selected from nitrogen, oxygen, or sulfur;
R¹ and R² are each independently halogen, R, OR, a suitably protected hydroxyl group, SR, a suitably protected thiol group, N(R)₂, or a suitably protected amino group, or R¹ and R² are taken together to form a 3-7 membered saturated or partially unsaturated ring having 0-2 heteroatoms independently selected from nitrogen, oxygen, or sulfur;
each R is independently deuterium, hydrogen, an optionally substituted C₁₋₆ aliphatic group, or an optionally substituted 3-8 membered saturated, partially unsaturated, or aryl ring having 0-4 heteroatoms independently selected from nitrogen, oxygen, or sulfur, wherein:
two R on the same nitrogen atom are optionally taken together with said nitrogen atom to form an optionally substituted 3-8 membered, saturated, partially unsaturated, or aryl ring having 1-4 heteroatoms independently selected from nitrogen, oxygen, or sulfur;
n is 0-4;
R³, R⁴, and R⁸ are each independently selected from halogen, CN, R, OR, a suitably protected hydroxyl group, SR, a suitably protected thiol group, S(O)R, SO₂R, OSO₂R, N(R)₂, a suitably protected amino group, N(R)C(O)R, N(R)C(O)C(O)R, N(R)C(O)N(R)₂, N(R)C(O)OR, C(O)OR, OC(O)R, C(O)N(R)₂, or OC(O)N(R)₂, or:
two R⁴ on the same carbon are optionally taken together to form an optionally substituted 3-8 membered saturated or partially unsaturated spirofused ring having 0-4 heteroatoms independently selected from nitrogen, oxygen, or sulfur, or:
two R⁴ on the same carbon are optionally taken together to form an oxo moiety, an oxime, an optionally substituted hydrazone, an optionally substituted imine, or an optionally substituted C₂₋₆ alkylidene;
m is 0-4;
each R⁵ is independently T-C(R′)₃, T-C(R′)₂C(R″)₃, OR, a suitably protected hydroxyl group, SR, a suitably protected thiol group, S(O)R, SO₂R, OSO₂R, N(R)₂, a suitably protected amino group, N(R)C(O)R, N(R)C(O)C(O)R, N(R)C(O)N(R)₂, N(R)C(O)OR, C(O)OR, OC(O)R, C(O)N(R)₂, or OC(O)N(R)₂, an optionally substituted 3-8 membered saturated, partially unsaturated, or aryl monocyclic ring having 0-4 heteroatoms independently selected from nitrogen, oxygen, or sulfur, an optionally substituted 8-10 membered saturated, partially unsaturated, or aryl bicyclic ring having 0-4 heteroatoms independently selected from nitrogen, oxygen, or sulfur, or:
two R⁵ on the same carbon are optionally taken together to form an oxo moiety, an oxime, an optionally substituted hydrazone, an optionally substituted imine, an optionally substituted C₂₋₆ alkylidene, or an optionally substituted 3-8 membered saturated or partially unsaturated spirocycle having 0-4 heteroatoms independently selected from nitrogen, oxygen, or sulfur;
each T is independently a valence bond or an optionally substituted straight or branched, saturated or unsaturated, C₁₋₆ alkylene chain wherein up to two methylene units of T are optionally and independently replaced by —O—, —N(R)—, —S—, —C(O)—, —S(O)—, or —S(O)₂—;
each R′ and R″ is independently selected from halogen, R, OR, SR, S(O)R, SO₂R, OSO₂R, N(R)₂, N(R)C(O)R, N(R)C(O)C(O)R, N(R)C(O)N(R)₂, N(R)C(O)OR, N(R)S(O)R, N(R)SO₂R, N(R)SO₂ORC(O)OR, OC(O)R, C(O)N(R)₂, OC(O)N(R)₂, or an optionally substituted 3-8 membered saturated, partially unsaturated, or aryl monocyclic ring having 0-4 heteroatoms independently selected from nitrogen, oxygen, or sulfur, or an optionally substituted 8-10 membered saturated, partially unsaturated, or aryl bicyclic ring having 0-4 heteroatoms independently selected from nitrogen, oxygen, or sulfur, or:
two R′ are optionally taken together to form an oxo moiety, an oxime, an optionally substituted hydrazone, an optionally substituted imine, an optionally substituted C₂₋₆ alkylidene, or an optionally substituted 3-8 membered saturated or partially unsaturated ring having 0-4 heteroatoms independently selected from nitrogen, oxygen, or sulfur, or:
two R″ are optionally taken together to form an oxo moiety, an oxime, an optionally substituted hydrazone, an optionally substituted imine, an optionally substituted C₂₋₆ alkylidene, or an optionally substituted 3-8 membered saturated or partially unsaturated ring having 0-4 heteroatoms independently selected from nitrogen, oxygen, or sulfur;
R⁶ is halogen, R, OR, SR, S(O)R, SO₂R, OSO₂R, N(R)₂, N(R)C(O)R, N(R)C(O)C(O)R, N(R)C(O)N(R)₂, N(R)C(O)OR, C(O)OR, OC(O)R, C(O)N(R)₂, or OC(O)N(R)₂, or:
R⁶ and R⁵ are optionally taken together to form an optionally substituted 3-8 membered saturated, partially unsaturated, or aryl ring having 0-4 heteroatoms independently selected from nitrogen, oxygen, or sulfur;
each of R⁷ and R^{7′} is independently selected from halogen, CN, N₃, R, OR, a suitably protected hydroxyl group, SR, a suitably protected thiol group, S(O)R, SO₂R, OSO₂R, N(R)₂, a suitably protected amino group, NRC(O)R, NRC(O)C(O)R, N(R)C(O)N(R)₂, N(R)C(O)OR, C(O)OR, OC(O)R, C(O)N(R)₂, or OC(O)N(R)₂, or:
R⁷ and R^{7′} are taken together to form an oxo moiety, an oxime, an optionally substituted hydrazone, an optionally substituted imine, an optionally substituted C₂₋₆ alkylidene, or an optionally substituted 3-8 membered saturated or partially unsaturated spirocycle having 0-4 heteroatoms independently selected from nitrogen, oxygen, or sulfur, or:
R⁶ and R⁷ or R⁶ and R^{7′} are optionally taken together to form an optionally substituted 3-8 membered saturated or partially unsaturated ring having 0-4 heteroatoms selected from nitrogen, oxygen, or sulfur;
p is 0-4;
each R⁹ is independently selected from halogen, R, OR, SR, or N(R)₂, or:
two R⁹ on the same carbon are optionally taken together to form an optionally substituted 3-8 membered or partially unsaturated spirofused ring having 0-4 heteroatoms independently selected from nitrogen, oxygen, or sulfur, or:
two R⁹ on the same carbon atom are optionally taken together to form an oxo moiety, an oxime, an optionally substituted hydrazone, an optionally substituted imine, or an optionally substituted C₂₋₆ alkylidene;
Q is a valence bond or an optionally substituted C₁₋₁₀ alkylene chain wherein one, two, or three methylene units of Q are optionally and independently replaced by —O—, —N(R)—, —S—, —C(O)—, —OC(O)—, —C(O)O—, —OC(O)O—, —S(O)—, or —S(O)₂—, —OSO₂O—, —N(R)C(O)—, —C(O)N(R)—, —N(R)C(O)O—, —OC(O)NR—, —N(R)C(O)NR—, or —Cy-, wherein:
each —Cy- is independently a bivalent optionally substituted saturated, partially unsaturated, or aromatic monocyclic or bicyclic ring selected from a 6-10 membered arylene, a 5-10 membered heteroarylene having 1-4 heteroatoms independently selected from oxygen, nitrogen, or sulfur, a 3-8 membered carbocyclylene, or a 3-10 membered heterocyclylene having 1-4 heteroatoms independently selected from oxygen, nitrogen, or sulfur;
R¹⁰ is hydrogen, halogen, an optionally substituted C₁₋₁₀ aliphatic, a suitably protected hydroxyl group, a suitably protected thiol group, a suitably protected amino group, an optionally substituted 3-8 membered saturated, partially unsaturated, or aryl monocyclic ring having 0-4 heteroatoms independently selected from nitrogen, oxygen, or sulfur, an optionally substituted 8-10 membered saturated, partially unsaturated, or aryl bicyclic ring having 0-4 heteroatoms independently selected from nitrogen, oxygen, or sulfur, a detectable moiety, a polymer residue, a peptide, a sugar-containing or sugar-like moiety, or:
   wherein when R¹⁰ is a ring, R¹⁰ is optionally substituted at any substitutable carbon with 1-7 R¹¹ and at any substitutable nitrogen with R¹²;
each R¹¹ is independently halogen, R, OR, SR, N(R)₂, N(R)C(O)R, N(R)C(O)OR, N(R)C(O)N(R)₂, N(R)SO₂R, N(R)SO₂OR, S(O)R, SO₂R, OSO₂R, C(O)R, CO₂R, OCO₂R, C(O)N(R)₂, or OC(O)N(R)₂, or wherein:
   two R¹¹ are optionally taken together to form an oxo moiety, an oxime, an optionally substituted hydrazone, an optionally substituted imine, an optionally substituted C₂₋₆ alkylidene, or an optionally substituted 3-8 membered saturated or partially unsaturated fused or spirofused ring having 0-4 heteroatoms independently selected from nitrogen, oxygen, or sulfur; and
each R¹² is independently R, OR, S(O)R, SO₂R, OSO₂R, C(O)R, CO₂R, OCO₂R, C(O)N(R)₂, or OC(O)N(R)₂, an optionally substituted aliphatic group, a suitably protected amino group, an optionally substituted 3-8 membered saturated, partially unsaturated, or aryl monocyclic ring having 0-4 heteroatoms independently selected from nitrogen, oxygen, or sulfur, an optionally substituted 8-10 membered saturated, partially unsaturated, or aryl bicyclic ring having 0-4 heteroatoms independently selected from nitrogen, oxygen, or sulfur, or wherein: R¹² and R¹¹ are optionally taken together to form an optionally substituted 3-8 membered saturated or partially unsaturated fused ring having 0-4 heteroatoms independently selected from nitrogen, oxygen, or sulfur.

### 2. Definitions

Compounds of this invention include those described generally above, and are further illustrated by the embodiments, sub-embodiments, and species disclosed herein. As used herein, the following definitions shall apply unless otherwise indicated. For purposes of this invention, the chemical elements are identified in accordance with the Periodic Table of the Elements, CAS version, Handbook of Chemistry and Physics, 75^{th} Ed. Additionally, general principles of organic chemistry are described in “Organic Chemistry,” Thomas Sorrell, University Science Books, Sausalito: 1999, and “March's Advanced Organic Chemistry,” 5^{th} Ed., Ed.: Smith, M. B. and March, J., John Wiley & Sons, New York: 2001, the entire contents of which are hereby incorporated by reference.

As defined generally above, each of Ring A, Ring B, Ring C, Ring D, and Ring E is independently saturated, partially unsaturated or aromatic. It will be appreciated that compounds of the present invention are contemplated as chemically feasible compounds. Accordingly, it will be understood by one of ordinary skill in the art that when any of Ring A, Ring B, Ring C, Ring D, and Ring E is unsaturated, then certain substituents on that ring will be absent in order to satisfy general rules of valency. For example, if Ring D is unsaturated at the bond between Ring D and Ring E, then R⁶ will be absent. Alternatively, if Ring D is unsaturated at the bond between Ring D and Ring C, then R⁸ and R³ will be absent. All combinations of saturation and unsaturation of any of Ring A, Ring B, Ring C, Ring D, and Ring E are contemplated by the present invention. Thus, in order to satisfy general rules of valency, and depending on the degree of saturation or unsaturation of any of Ring A, Ring B, Ring C, Ring D, and Ring E, the requisite presence or absence of each of R¹, R², R³, R⁴, R⁵, R⁶, R⁷, R^{7′}, R⁸, R⁹, Q, and R¹⁰ is contemplated accordingly.

As described herein, compounds of the invention may optionally be substituted with one or more substituents, such as are illustrated generally above, or as exemplified by particular classes, subclasses, and species of the invention. It will be appreciated that the phrase “optionally substituted” is used interchangeably with the phrase “substituted or unsubstituted.” In general, the term “substituted,” whether preceded by the term “optionally” or not, refers to the replacement of hydrogen radicals in a given structure with the radical of a specified substituent. Unless otherwise indicated, an optionally substituted group may have a substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds.

The term “stable,” as used herein, refers to compounds that are not substantially altered when subjected to conditions to allow for their production, detection, and preferably their recovery, purification, and use for one or more of the purposes disclosed herein. In some embodiments, a stable compound or chemically feasible compound is one that is not substantially altered when kept at a temperature of 40° C. or less, in the absence of moisture or other chemically reactive conditions, for at least a week.

The term “aliphatic” or “aliphatic group,” as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle” “cycloaliphatic” or “cycloalkyl”), that has a single point of attachment to the rest of the molecule. Unless otherwise specified, aliphatic groups contain 1-20 aliphatic carbon atoms. In some embodiments, aliphatic groups contain 1-6 aliphatic carbon atoms. In yet other embodiments aliphatic groups contain 1-4 aliphatic carbon atoms. In some embodiments, “cycloaliphatic” (or “carbocycle” or “cycloalkyl”) refers to a monocyclic C₃-C₈ hydrocarbon or bicyclic C₈-C₁₂ hydrocarbon that is completely saturated or that contains one or more units of unsaturation, but which is not aromatic, that has a single point of attachment to the rest of the molecule wherein any individual ring in said bicyclic ring system has 3-7 members. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl. In other embodiments, an aliphatic group may have two geminal hydrogen atoms replaced with oxo (a bivalent carbonyl oxygen atom ═O), or a ring-forming substituent, such as —O-(straight or branched alkylene or alkylene)-O— to form an acetal or ketal.

In certain embodiments, exemplary aliphatic groups include, but are not limited to, ethynyl, 2-propynyl, 1-propenyl, 2-butenyl, 1,3-butadienyl, 2-pentenyl, vinyl (ethenyl), allyl, isopropenyl, methyl, ethyl, propyl, isopropyl, butyl, isobutyl, sec-butyl, tert-butyl, pentyl, isopentyl, sec-pentyl, neo-pentyl, tert-pentyl, cyclopentyl, hexyl, isohexyl, sec-hexyl, cyclohexyl, 2-methylpentyl, tert-hexyl, 2,3-dimethylbutyl, 3,3-dimethylbutyl, 1,3-dimethylbutyl, and 2,3-dimethyl but-2-yl.

The term “alkylene,” as used herein, refers to a bivalent straight or branched saturated or unsaturated hydrocarbon chain. In some embodiments, an alkylene group is saturated. In some embodiments, an alkylene group contains one or more units of unsaturation.

The term “alkylidene,” as used herein, refers to a divalent group formed from an alkane by removal of two hydrogen atoms from the same carbon atom, the free valencies of which are part of a double bond. By way of nonlimiting example, an alkylidene may be of the formula ═C(R^{q})₂, ═CHR^{q}, or ═CH₂, wherein R^{q} represents any suitable substituent other than hydrogen.

The terms “haloalkyl,” “haloalkenyl” and “haloalkoxy” means alkyl, alkenyl or alkoxy, as the case may be, substituted with one or more halogen atoms. The term “halogen” means F, Cl, Br, or I. Such “haloalkyl,” “haloalkenyl” and “haloalkoxy” groups may have two or more halo substituents which may or may not be the same halogen and may or may not be on the same carbon atom. Examples include chloromethyl, periodomethyl, 3,3-dichloropropyl, 1,3-difluorobutyl, trifluoromethyl, and 1-bromo-2-chloropropyl.

The term “heterocycle,” “heterocyclyl,” “heterocycloaliphatic,” or “heterocyclic” as used herein means non-aromatic, monocyclic, bicyclic, or tricyclic ring systems in which one or more ring members is an independently selected heteroatom. In some embodiments, the “heterocycle,” “heterocyclyl,” “heterocycloaliphatic,” or “heterocyclic” group has three to fourteen ring members in which one or more ring members is a heteroatom independently selected from oxygen, sulfur, nitrogen, or phosphorus, and each ring in the system contains 3 to 7 ring members.

A heterocyclic ring can be attached to its pendant group at any heteroatom or carbon atom that results in a stable structure and, when specified, any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR⁺ (as in N-substituted pyrrolidinyl).

The term “unsaturated,” as used herein, means that a moiety has one or more units of unsaturation.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

The term “alkoxy,” or “thioalkyl,” as used herein, refers to an alkyl group, as previously defined, attached to the principal carbon chain through an oxygen (“alkoxy”) or sulfur (“thioalkyl”) atom.

The term “aryl” used alone or as part of a larger moiety as in “aralkyl,” “aralkoxy,” or “aryloxyalkyl,” refers to monocyclic, bicyclic, and tricyclic ring systems having a total of five to fourteen ring members, wherein one or more ring in the system is aromatic and wherein each ring in the system contains 3 to 7 ring members. The term “aryl” may be used interchangeably with the term “aryl ring”. The term “aryl” also refers to heteroaryl ring systems as defined hereinbelow. In certain embodiments of the present invention, “aryl” refers to an aromatic ring system which includes, but not limited to, phenyl, biphenyl, naphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like.

The term “heteroaryl,” used alone or as part of a larger moiety as in “heteroaralkyl” or “heteroarylalkoxy,” refers to monocyclic, bicyclic, and tricyclic ring systems having a total of five to fourteen ring members, wherein one or more ring in the system is aromatic, one or more ring in the system contains one or more heteroatoms, and wherein each ring in the system contains 3 to 7 ring members. The term “heteroaryl” may be used interchangeably with the term “heteroaryl ring” or the term “heteroaromatic”. Heteroaryl groups include thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl.

The terms “heteroaryl” and “heteroar-,” as used herein, also include groups in which a heteroaromatic ring is fused to one or more aryl, cycloaliphatic, or heterocyclyl rings. Exemplary heteroaryl rings include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one.

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₄R^{o}; —(CH₂)₀₋₄OR^{o}; —O(CH₂)₀₋₄R^{o}, —O—(CH₂)₀₋₄C(O)OR^{o}; —(CH₂)₀₋₄CH(OR^{o)}₂; —(CH₂)₀₋₄SR^{o}; —(CH₂)₀₋₄Ph, which may be substituted with R^{o}; —(CH₂)₀₋₄O(CH₂)₀₋₄O(CH₂)₀₋₁Ph which may be substituted with R^{o}; —CH═CHPh, which may be substituted with R^{o}; —(CH₂)₀₋₄O(CH₂)₀₋₁-Pyridyl which may be substituted with R^{o}; —NO₂; —CN; —N₃; —(CH₂)₀₋₄N(R^{o})₂; —(CH₂)₀₋₄N(R^{o})C(O)R^{o}; —N(R^{o})C(S)R^{o}; —(CH₂)₀₋₄N(R^{o})C(O)NR^{o}₂; —N(R^{o})C(S)NR^{o}₂; —(CH₂)₀₋₄N(R^{o})C(O)OR^{o}; —N(R^{o})N(R^{o})C(O)R^{o}; —N(R^{o}N(R^{o})C(O)NR^{o})₂; —N(R^{o}N(R^{o}C(O)OR^{o}; —(CH₂)₀₋₄C(O)R^{o}; —C(S)R^{o}; —(CH₂)₀₋₄C(O)OR^{o}; —(CH₂)₀₋₄C(O)SR^{o}; —(CH₂)₀₋₄C(O)OSiR^{o}₃; —(CH₂)₀₋₄OC(O)R^{o}; —OC(O)(CH₂)₀₋₄SR^{o}, SC(S)SR^{o}; —(CH₂)₀₋₄SC(O)R^{o}; —(CH₂)₀₋₄C(O)NR^{o}₂; —C(S)NR^{o}₂; —C(S)SR^{o}; —SC(S)SR^{o}, —(CH₂)₀₋₄OC(O)NR^{o})₂; —C(O)N(OR^{o})R^{o}; —C(O)C(O)R^{o}; —C(O)CH₂C(O)R^{o}; —C(NOR^{o})R^{o}; —(CH₂)₀₋₄SSR^{o}; —(CH₂)₀₋₄S(O)₂R^{o}; —(CH₂)₀₋₄S(O)₂OR^{o}; —(CH₂)₀₋₄OS(O)₂R^{o}; —S(O)₂NR^{o}₂; —(CH₂)₀₋₄S(O)R^{o}; —N(R^{o})S(O)₂NR^{o}₂; —N(R^{o})S(O)₂R^{o}; —N(OR^{o}R^{o}; —C(NH)NR^{o}₂; —P(O)₂R^{o}; —P(O)R^{o}₂; —OP(O)R^{o}₂; —OP(O)(OR^{o)}₂; SiR^{o}₃; —(C₁₋₄ straight or branched)alkylene)O—N(R^{o})₂; or —(C₁₋₄ straight or branched)alkylene)C(O)O—N(R^{o})₂, wherein each R^{o} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{o}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{o} (or the ring formed by taking two independent occurrences of R^{o} together with their intervening atoms), are independently halogen, —(CH₂)₀₋₂R^{}, -(haloR^{}), —(CH₂)₀₋₂OH, —(CH₂)₀₋₂OR^{}, —(CH₂)₀₋₂CH(OR^{})₂; —O(haloR^{}), —CN, —N₃, —(CH₂)₀₋₂C(O)R^{}, —(CH₂)₀₋₂C(O)OH, —(CH₂)₀₋₂C(O)OR^{}, —(CH₂)₀₋₂SR^{}, —(CH₂)₀₋₂SH, —(CH₂)₀₋₂NH₂, —(CH₂)₀₋₂NHR^{}, —(CH₂)₀₋₂NR^{}₂, —NO₂, —SiR^{}₃, —OSiR^{}₃, —C(O)SR^{}, —(C₁₋₄ straight or branched alkylene)C(O)OR^{}, or —SSR^{} wherein each R^{} is unsubstituted or where preceded by “halo” is substituted only with one or more halogens, and is independently selected from C₁ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{o} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, and ═C(R*)₂, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{}, -(haloR^{}), —OH, —OR^{}, —O(haloR^{}), —CN, —C(O)OH, —C(O)OR^{}, —NH₂, —NHR^{}, —NR^{}₂, or —NO₂, wherein each R^{} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{}, -(haloR^{}), —OH, —OR^{}, —O(haloR^{}), —CN, —C(O)OH, —C(O)OR^{}, —NH₂, —NHR^{}, —NR^{}₂, or —NO₂, wherein each R^{} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

As used herein, the term “detectable moiety” is used interchangeably with the term “label” and relates to any moiety capable of being detected, e.g., primary labels and secondary labels. Primary labels, such as radioisotopes (e.g., ³²P, ³³P, ³⁵S, or ¹⁴C), mass-tags, and fluorescent labels are signal generating reporter groups which can be detected without further modifications.

The term “secondary label” as used herein refers to moieties such as biotin and various protein antigens that require the presence of a second intermediate for production of a detectable signal. For biotin, the secondary intermediate may include streptavidin-enzyme conjugates. For antigen labels, secondary intermediates may include antibody-enzyme conjugates. Some fluorescent groups act as secondary labels because they transfer energy to another group in the process of nonradiative fluorescent resonance energy transfer (FRET), and the second group produces the detected signal.

The terms “fluorescent label,” “fluorescent dye,” and “fluorophore” as used herein refer to moieties that absorb light energy at a defined excitation wavelength and emit light energy at a different wavelength. Examples of fluorescent labels include, but are not limited to: Alexa Fluor dyes (Alexa Fluor 350, Alexa Fluor 488, Alexa Fluor 532, Alexa Fluor 546, Alexa Fluor 568, Alexa Fluor 594, Alexa Fluor 633, Alexa Fluor 660, and Alexa Fluor 680), AMCA, AMCA-S, BODIPY dyes (BODIPY FL, BODIPY R6G, BODIPY TMR, BODIPY TR, BODIPY 530/550, BODIPY 558/568, BODIPY 564/570, BODIPY 576/589, BODIPY 581/591, BODIPY 630/650, and BODIPY 650/665), Carboxyrhodamine 6G, carboxy-X-rhodamine (ROX), Cascade Blue, Cascade Yellow, Coumarin 343, Cyanine dyes (Cy3, Cy5, Cy3.5, and Cy5.5), Dansyl, Dapoxyl, Dialkylaminocoumarin, 4′,5′-Dichloro-2′,7′-dimethoxy-fluorescein, DM-NERF, Eosin, Erythrosin, Fluorescein, FAM, Hydroxycoumarin, IRDyes (IRD40, IRD 700, and IRD 800), JOE, Lissamine rhodamine B, Marina Blue, Methoxycoumarin, Naphthofluorescein, Oregon Green 488, Oregon Green 500, Oregon Green 514, Pacific Blue, PyMPO, Pyrene, Rhodamine B, Rhodamine 6G, Rhodamine Green, Rhodamine Red, Rhodol Green, 2′,4′,5′,7′-Tetra-bromosulfone-fluorescein, Tetramethyl-rhodamine (TMR), Carboxytetramethylrhodamine (TAMRA), Texas Red, and Texas Red-X.

The term “mass-tag” as used herein refers to any moiety that is capable of being uniquely detected by virtue of its mass using mass spectrometry (MS) detection techniques. Examples of mass-tags include electrophore release tags such as N-[3-[4′-[(p-methoxytetrafluorobenzyl)oxy]phenyl]-3-methylglyceronyl]isonipecotic acid, 4′42,3,5,6-tetrafluoro-4-(pentafluorophenoxyl)]methyl acetophenone, and their derivatives. The synthesis and utility of these mass-tags is described in U.S. Pat. Nos. 4,650,750, 4,709,016, 5,360,8191, 5,516,931, 5,602,273, 5,604,104, 5,610,020, and 5,650,270. Other examples of mass-tags include, but are not limited to, nucleotides, dideoxynucleotides, oligonucleotides of varying length and base composition, oligopeptides, oligosaccharides, and other synthetic polymers of varying length and monomer composition. A large variety of organic molecules, both neutral and charged (biomolecules or synthetic compounds) of an appropriate mass range (100-2000 Daltons) may also be used as mass-tags.

The term “substrate,” as used herein refers to any material or macromolecular complex to which a functionalized end-group of a compound of the present invention can be attached. Examples of commonly used substrates include, but are not limited to, glass surfaces, silica surfaces, plastic surfaces, metal surfaces, surfaces containing a metallic or chemical coating, membranes (e.g., nylon, polysulfone, or silica), micro-beads (e.g., latex, polystyrene, or other polymer), porous polymer matrices (e.g., polyacrylamide gel, polysaccharide, or polymethacrylate), and macromolecular complexes (e.g., protein, or polysaccharide).

Unless otherwise stated, structures depicted herein are also meant to include all isomeric (e.g., enantiomeric, diastereomeric, and geometric (or conformational)) forms of the structure; for example, the R and S configurations for each asymmetric center, (Z) and (E) double bond isomers, and (Z) and (E) conformational isomers. Therefore, single stereochemical isomers as well as enantiomeric, diastereomeric, and geometric (or conformational) mixtures of the present compounds are within the scope of the invention.

Unless otherwise stated, all tautomeric forms of the compounds of the invention are within the scope of the invention.

Additionally, unless otherwise stated, structures depicted herein are also meant to include compounds that differ only in the presence of one or more isotopically enriched atoms. For example, compounds having the present structures except for the replacement of hydrogen by deuterium or tritium, or the replacement of a carbon by a ¹¹C- or ¹³C- or ¹⁴C-enriched carbon are within the scope of this invention. Such compounds are useful, for example, as analytical tools or probes in biological assays.

### 3. Description of Exemplary Compounds

In some embodiments, the present invention provides a compound of formula I:

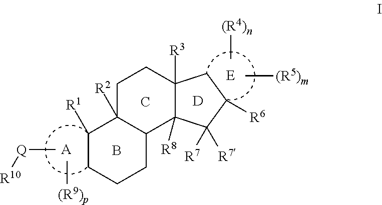

or a pharmaceutically acceptable salt thereof, wherein:
Ring A is a 4-7 membered saturated or partially unsaturated ring having 0-2 heteroatoms independently selected from nitrogen, oxygen, or sulfur;
each of Ring B, Ring C, and Ring D is independently saturated, partially unsaturated or aromatic, or a deuterated derivative thereof;
Ring E is a 4-7 membered saturated, partially unsaturated, or aromatic ring having 0-2 heteroatoms independently selected from nitrogen, oxygen, or sulfur;
R¹ and R² are each independently halogen, R, OR, a suitably protected hydroxyl group, SR, a suitably protected thiol group, N(R)₂, or a suitably protected amino group, or R¹ and R² are taken together to form a 3-7 membered saturated or partially unsaturated ring having 0-2 heteroatoms independently selected from nitrogen, oxygen, or sulfur;
each R is independently deuterium, hydrogen, an optionally substituted C₁₋₆ aliphatic group, or an optionally substituted 3-8 membered saturated, partially unsaturated, or aryl ring having 0-4 heteroatoms independently selected from nitrogen, oxygen, or sulfur, wherein:
two R on the same nitrogen atom are optionally taken together with said nitrogen atom to form an optionally substituted 3-8 membered, saturated, partially unsaturated, or aryl ring having 1-4 heteroatoms independently selected from nitrogen, oxygen, or sulfur;
n is 0-4;
R³, R⁴, and R⁸ are each independently selected from halogen, CN, R, OR, a suitably protected hydroxyl group, SR, a suitably protected thiol group, S(O)R, SO₂R, OSO₂R, N(R)₂, a suitably protected amino group, N(R)C(O)R, N(R)C(O)C(O)R, N(R)C(O)N(R)₂, N(R)C(O)OR, C(O)OR, OC(O)R, C(O)N(R)₂, or OC(O)N(R)₂, or:
two R⁴ on the same carbon are optionally taken together to form an optionally substituted 3-8 membered saturated or partially unsaturated spirofused ring having 0-4 heteroatoms independently selected from nitrogen, oxygen, or sulfur, or:
two R⁴ on the same carbon are optionally taken together to form an oxo moiety, an oxime, an optionally substituted hydrazone, an optionally substituted imine, or an optionally substituted C₂₋₆ alkylidene;
m is 0-4;
each R⁵ is independently T-C(R′)₃, T-C(R′)₂C(R″)₃, OR, a suitably protected hydroxyl group, SR, a suitably protected thiol group, S(O)R, SO₂R, OSO₂R, N(R)₂, a suitably protected amino group, N(R)C(O)R, N(R)C(O)C(O)R, N(R)C(O)N(R)₂, N(R)C(O)OR, C(O)OR, OC(O)R, C(O)N(R)₂, or OC(O)N(R)₂, an optionally substituted 3-8 membered saturated, partially unsaturated, or aryl monocyclic ring having 0-4 heteroatoms independently selected from nitrogen, oxygen, or sulfur, an optionally substituted 8-10 membered saturated, partially unsaturated, or aryl bicyclic ring having 0-4 heteroatoms independently selected from nitrogen, oxygen, or sulfur, or:
two R⁵ on the same carbon are optionally taken together to form an oxo moiety, an oxime, an optionally substituted hydrazone, an optionally substituted imine, an optionally substituted C₂₋₆ alkylidene, or an optionally substituted 3-8 membered saturated or partially unsaturated spirocycle having 0-4 heteroatoms independently selected from nitrogen, oxygen, or sulfur;
each T is independently a valence bond or an optionally substituted straight or branched, saturated or unsaturated, C₁₋₆ alkylene chain wherein up to two methylene units of T are optionally and independently replaced by —O—, —N(R)—, —S—, —C(O)—, —S(O)—, or —S(O)₂—;
each R′ and R″ is independently selected from halogen, R, OR, SR, S(O)R, SO₂R, OSO₂R, N(R)₂, N(R)C(O)R, N(R)C(O)C(O)R, N(R)C(O)N(R)₂, N(R)C(O)OR, N(R)S(O)R, N(R)SO₂R, N(R)SO₂OR C(O)OR, OC(O)R, C(O)N(R)₂, OC(O)N(R)₂, or an optionally substituted 3-8 membered saturated, partially unsaturated, or aryl monocyclic ring having 0-4 heteroatoms independently selected from nitrogen, oxygen, or sulfur, or an optionally substituted 8-10 membered saturated, partially unsaturated, or aryl bicyclic ring having 0-4 heteroatoms independently selected from nitrogen, oxygen, or sulfur, or:
two R′ are optionally taken together to form an oxo moiety, an oxime, an optionally substituted hydrazone, an optionally substituted imine, an optionally substituted C₂₋₆ alkylidene, or an optionally substituted 3-8 membered saturated or partially unsaturated ring having 0-4 heteroatoms independently selected from nitrogen, oxygen, or sulfur, or:
two R″ are optionally taken together to form an oxo moiety, an oxime, an optionally substituted hydrazone, an optionally substituted imine, an optionally substituted C₂₋₆ alkylidene, or an optionally substituted 3-8 membered saturated or partially unsaturated ring having 0-4 heteroatoms independently selected from nitrogen, oxygen, or sulfur;
R⁶ is halogen, R, OR, SR, S(O)R, SO₂R, OSO₂R, N(R)₂, N(R)C(O)R, N(R)C(O)C(O)R, N(R)C(O)N(R)₂, N(R)C(O)OR, C(O)OR, OC(O)R, C(O)N(R)₂, or OC(O)N(R)₂, or:
R⁶ and R⁵ are optionally taken together to form an optionally substituted 3-8 membered saturated, partially unsaturated, or aryl ring having 0-4 heteroatoms independently selected from nitrogen, oxygen, or sulfur;
each of R⁷ and R^{7′} is independently selected from halogen, CN, N₃, R, OR, a suitably protected hydroxyl group, SR, a suitably protected thiol group, S(O)R, SO₂R, OSO₂R, N(R)₂, a suitably protected amino group, NRC(O)R, NRC(O)C(O)R, N(R)C(O)N(R)₂, N(R)C(O)OR, C(O)OR, OC(O)R, C(O)N(R)₂, or OC(O)N(R)₂, or:
R⁷ and R^{7′} are taken together to form an oxo moiety, an oxime, an optionally substituted hydrazone, an optionally substituted imine, an optionally substituted C₂₋₆ alkylidene, or an optionally substituted 3-8 membered saturated or partially unsaturated spirocycle having 0-4 heteroatoms independently selected from nitrogen, oxygen, or sulfur, or:
R⁶ and R⁷ or R⁶ and R^{7′} are optionally taken together to form an optionally substituted 3-8 membered saturated or partially unsaturated ring having 0-4 heteroatoms selected from nitrogen, oxygen, or sulfur;
p is 0-4;
each R⁹ is independently selected from halogen, R, OR, SR, or N(R)₂, or:
two R⁹ on the same carbon are optionally taken together to form an optionally substituted 3-8 membered or partially unsaturated spirofused ring having 0-4 heteroatoms independently selected from nitrogen, oxygen, or sulfur, or:
two R⁹ on the same carbon atom are optionally taken together to form an oxo moiety, an oxime, an optionally substituted hydrazone, an optionally substituted imine, or an optionally substituted C₂₋₆ alkylidene;
Q is a valence bond or an optionally substituted C₁₋₁₀ alkylene chain wherein one, two, or three methylene units of Q are optionally and independently replaced by —O—, —N(R)—, —S—, —C(O)—, —OC(O)—, —C(O)O—, —OC(O)O—, —S(O)—, or —S(O)₂—, —OSO₂O—, —N(R)C(O)—, —C(O)N(R)—, —N(R)C(O)O—, —OC(O)NR—, —N(R)C(O)NR—, or —Cy-, wherein:
each —Cy- is independently a bivalent optionally substituted saturated, partially unsaturated, or aromatic monocyclic or bicyclic ring selected from a 6-10 membered arylene, a 5-10 membered heteroarylene having 1-4 heteroatoms independently selected from oxygen, nitrogen, or sulfur, a 3-8 membered carbocyclylene, or a 3-10 membered heterocyclylene having 1-4 heteroatoms independently selected from oxygen, nitrogen, or sulfur;
R¹⁰ is hydrogen, halogen, an optionally substituted C₁₋₁₀ aliphatic, a suitably protected hydroxyl group, a suitably protected thiol group, a suitably protected amino group, an optionally substituted 3-8 membered saturated, partially unsaturated, or aryl monocyclic ring having 0-4 heteroatoms independently selected from nitrogen, oxygen, or sulfur, an optionally substituted 8-10 membered saturated, partially unsaturated, or aryl bicyclic ring having 0-4 heteroatoms independently selected from nitrogen, oxygen, or sulfur, a detectable moiety, a polymer residue, a peptide, a sugar-containing or sugar-like moiety, or:
   wherein when R¹⁰ is a ring, R¹⁰ is optionally substituted at any substitutable carbon with 1-7 R¹¹ and at any substitutable nitrogen with R¹²;
each R¹¹ is independently halogen, R, OR, SR, N(R)₂, N(R)C(O)R, N(R)C(O)OR, N(R)C(O)N(R)₂, N(R)SO₂R, N(R)SO₂OR, S(O)R, SO₂R, OSO₂R, C(O)R, CO₂R, OCO₂R, C(O)N(R)₂, or OC(O)N(R)₂, or wherein:
   two R¹¹ are optionally taken together to form an oxo moiety, an oxime, an optionally substituted hydrazone, an optionally substituted imine, an optionally substituted C₂₋₆ alkylidene, or an optionally substituted 3-8 membered saturated or partially unsaturated fused or spirofused ring having 0-4 heteroatoms independently selected from nitrogen, oxygen, or sulfur; and
each R¹² is independently R, OR, S(O)R, SO₂R, OSO₂R, C(O)R, CO₂R, OCO₂R, C(O)N(R)₂, or OC(O)N(R)₂, an optionally substituted aliphatic group, a suitably protected amino group, an optionally substituted 3-8 membered saturated, partially unsaturated, or aryl monocyclic ring having 0-4 heteroatoms independently selected from nitrogen, oxygen, or sulfur, an optionally substituted 8-10 membered saturated, partially unsaturated, or aryl bicyclic ring having 0-4 heteroatoms independently selected from nitrogen, oxygen, or sulfur, or wherein: R¹² and R¹¹ are optionally taken together to form an optionally substituted 3-8 membered saturated or partially unsaturated fused ring having 0-4 heteroatoms independently selected from nitrogen, oxygen, or sulfur.

### 4. Embodiments of R¹ and R²

As defined generally above, R¹ and R² of formula I are each independently halogen, R, OR, a suitably protected hydroxyl group, SR, a suitably protected thiol group, N(R)₂, or a suitably protected amino group, or R¹ and R² are taken together to form a 3-7 membered saturated, partially unsaturated, or aryl ring having 0-2 heteroatoms independently selected from nitrogen, oxygen, or sulfur. In certain embodiments, R¹ and R² of formula I are each independently R or OR. In other embodiments, R¹ and R² of formula I are each independently R, wherein R is hydrogen or an optionally substituted C₁₋₆ aliphatic group. According to another aspect of the present invention, R¹ and R² of formula I are taken together to form a 3-6 membered saturated, partially unsaturated, or aryl ring having 0-2 heteroatoms independently selected from nitrogen, oxygen, or sulfur. Yet another aspect of the present invention provides a compound of formula I wherein R¹ and R² are taken together to form a 3-6 membered saturated carbocyclic ring. In other embodiments, R¹ and R² of formula I are taken together to form a cyclopropyl ring.

### 5. Stereochemistry Embodiments

As described generally above, the present invention provides a compound of formula I:

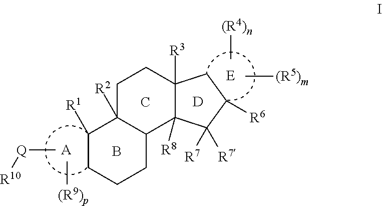

or a pharmaceutically acceptable salt thereof, wherein each variable is defined above and in classes and subclasses described above and herein.

In certain embodiments, the present invention provides a compound of formula I having the stereochemistry as depicted in formula I-a:

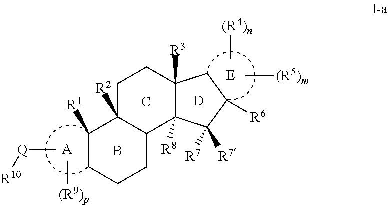

or a pharmaceutically acceptable salt thereof, wherein each variable is defined above and in classes and subclasses described above and herein for compounds of formula I.

In certain embodiments, the present invention provides a compound of formula I having the stereochemistry as depicted in formula I-b or I-c:

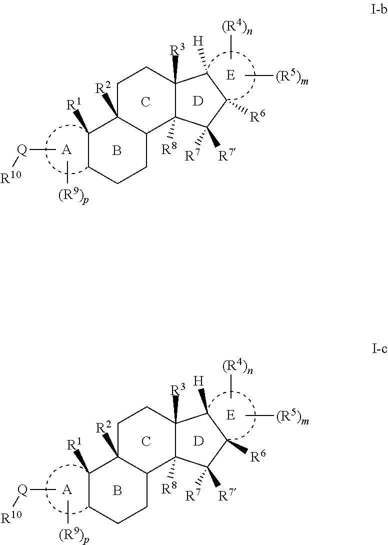

or a pharmaceutically acceptable salt thereof, wherein each variable is defined above and in classes and subclasses described above and herein for compounds of formula I.

In certain embodiments, the present invention provides a compound of formula I having the stereochemistry as depicted in formula I-d or I-e:

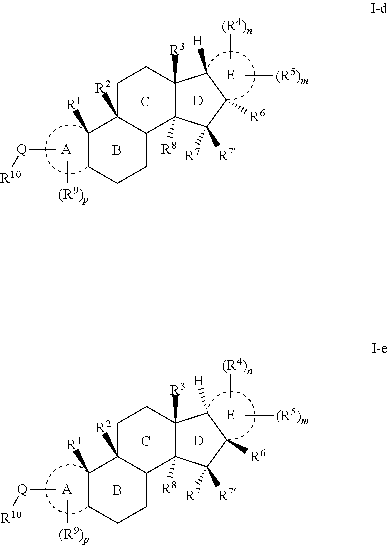

or a pharmaceutically acceptable salt thereof, wherein each variable is defined above and in classes and subclasses described above and herein for compounds of formula I.

In certain embodiments, the R¹ and R² groups of formula I are taken together to form a 3-7 membered saturated or partially unsaturated ring having 0-2 heteroatoms independently selected from nitrogen, oxygen, or sulfur. In other embodiments, the R¹ and R² groups of formula I are taken together to form a 3-6 membered saturated ring having 0-2 heteroatoms independently selected from nitrogen, oxygen, or sulfur. In still other embodiments, the R¹ and R² groups of formula I are taken together to form a 3-6 membered saturated carbocyclic ring. According to yet another aspect of the present invention, a compound of formula II is provided:

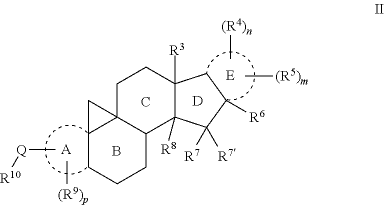

or a pharmaceutically acceptable salt thereof, wherein each variable is defined above and in classes and subclasses described above and herein for compounds of formula I.

In certain embodiments, the present invention provides a compound of formula II having the stereochemistry as depicted in formula II-a:

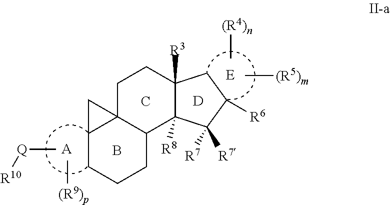

or a pharmaceutically acceptable salt thereof, wherein each variable is defined above and in classes and subclasses described above and herein for compounds of formula I.

In certain embodiments, the present invention provides a compound of formula II having the stereochemistry as depicted in formula II-b or II-c:

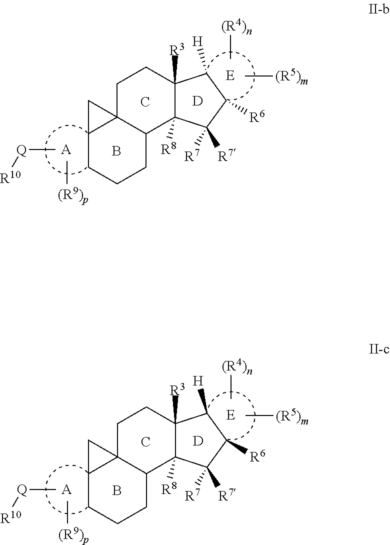

or a pharmaceutically acceptable salt thereof, wherein each variable is defined above and in classes and subclasses described above and herein for compounds of formula I.

In certain embodiments, the present invention provides a compound of formula II having the stereochemistry as depicted in formula II-d or II-e:

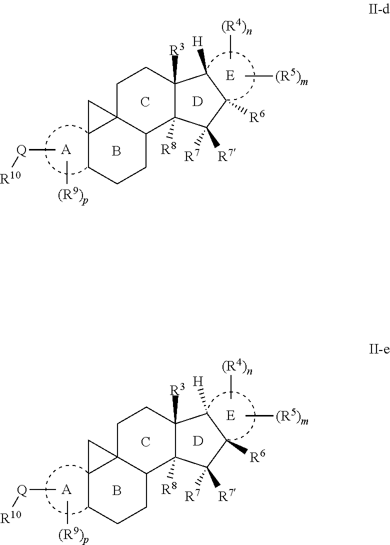

or a pharmaceutically acceptable salt thereof, wherein each variable is defined above and in classes and subclasses described above and herein for compounds of formula I.

In certain embodiments, the present invention provides a compound of formula II having the stereochemistry as depicted in formula II-f or II-g:

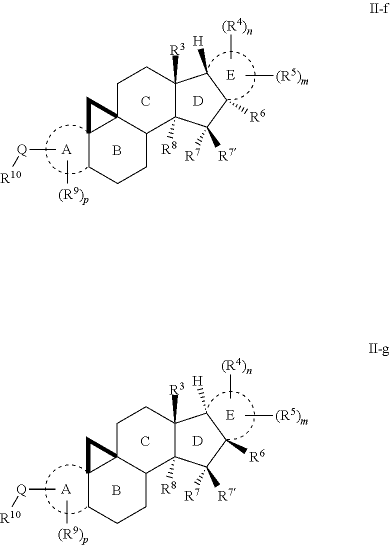

or a pharmaceutically acceptable salt thereof, wherein each variable is defined above and in classes and subclasses described above and herein for compounds of formula I.

In some embodiments, the present invention provides a compound of formula III:

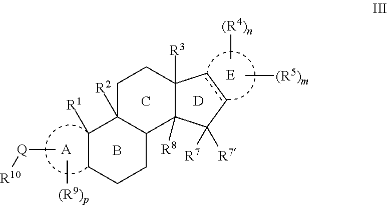

or a pharmaceutically acceptable salt thereof, wherein each variable is defined above and in classes and subclasses described above and herein for compounds of formula I. As used herein, [symbol] designates a single or double bond. It will be understood to one of ordinary skill in the art that when [symbol] designates a double bond, then R⁶ is absent. In contrast, when [symbol] designates a single bond, then R⁶ is present. Accordingly, in certain embodiments, [symbol] designates a double bond and R⁶ is absent. In other embodiments, [symbol] designates a single bond and R⁶ is as defined above.

In some embodiments, the present invention provides a compound of formula IV:

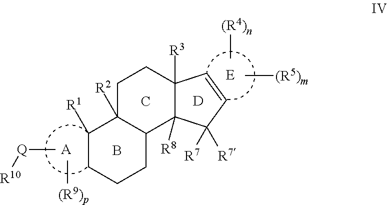

or a pharmaceutically acceptable salt thereof, wherein each variable is defined above and in classes and subclasses described above and herein for compounds of formula I.

In certain embodiments, the present invention provides a compound of formula I having the stereochemistry as depicted in formula IV-a:

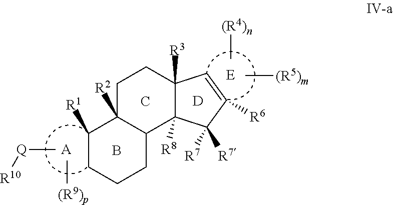

or a pharmaceutically acceptable salt thereof, wherein each variable is defined above and in classes and subclasses described above and herein for compounds of formula I.

In certain embodiments, the R¹ and R² groups of formula IV-a are taken together to form a 3-7 membered saturated or partially unsaturated ring having 0-2 heteroatoms independently selected from nitrogen, oxygen, or sulfur. In other embodiments, the R¹ and R² groups of formula IV-a are taken together to form a 3-6 membered saturated ring having 0-2 heteroatoms independently selected from nitrogen, oxygen, or sulfur. In still other embodiments, the R¹ and R² groups of formula IV-a are taken together to form a 3-6 membered saturated carbocyclic ring.

According to yet another aspect of the present invention, a compound of formula IV-b is provided:

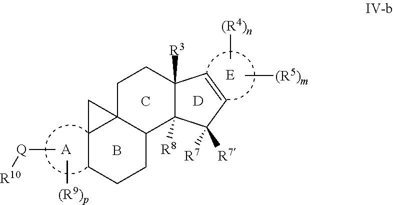

or a pharmaceutically acceptable salt thereof, wherein each variable is defined above and in classes and subclasses described above and herein for compounds of formula I.

In some embodiments, a compound of formula IV-c is provided:

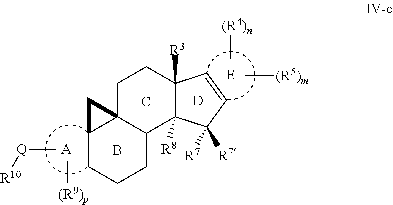

or a pharmaceutically acceptable salt thereof, wherein each variable is defined above and in classes and subclasses described above and herein for compounds of formula I.

### 6. Q, R¹⁰, R¹¹, and R¹² Embodiments

As defined generally above and herein, Q is a valence bond or an optionally substituted C₁₋₁₀ alkylene chain wherein one, two, or three methylene units of Q are optionally and independently replaced by —O—, —N(R)—, —S—, —C(O)—, —OC(O)—, —C(O)O—, —OC(O)O—, —S(O)—, or —S(O)₂—, —OSO₂O—, —N(R)C(O)—, —C(O)NR—, —N(R)C(O)O—, —OC(O)NR—, —N(R)C(O)NR—, or —Cy-, wherein:
each —Cy- is independently a bivalent optionally substituted saturated, partially unsaturated, or aromatic monocyclic or bicyclic ring selected from a 6-10 membered arylene, a 5-10 membered heteroarylene having 1-4 heteroatoms independently selected from oxygen, nitrogen, or sulfur, a 3-8 membered carbocyclylene, or a 3-10 membered heterocyclylene having 1-4 heteroatoms independently selected from oxygen, nitrogen, or sulfur.

In some embodiments, Q is a valence bond. In some embodiments, Q is an optionally substituted C₁₋₁₀ alkylene chain wherein one, two, or three methylene units are independently replaced by —O—, —N(R)—, —S—, —C(O)—, —OC(O)—, —C(O)O—, —OC(O)O—, —S(O)—, or —S(O)₂—, —OSO₂O—, —NRC(O)—, —C(O)NR—, —N(R)C(O)O—, —OC(O)NR—, —N(R)C(O)NR—, or —Cy-. In certain embodiments, Q is —O—. In certain embodiments, Q is —N(R)—. In certain embodiments, Q is —S—. In certain embodiments, Q is —N(Me)-.

In certain embodiments, Q is an optionally substituted C₁₋₁₀ alkylene chain wherein one, two, or three methylene units are independently replaced by —O—, —N(R)—, —S—, —C(O)—, —SO₂—, or —Cy-. In certain embodiments, Q is an optionally substituted C₂₋₁₀ alkylene chain wherein two or more methylene units are independently replaced by —O— and —Cy-. In certain embodiments, Q is an optionally substituted C₂₋₁₀ alkylene chain wherein two or more methylene units are independently replaced by —O— and —C(O)—. In certain embodiments, Q is an optionally substituted C₂₋₁₀ alkylene chain wherein two or more methylene units are independently replaced by —N(R)— and —C(O)—. In certain embodiments, Q is an optionally substituted C₂₋₁₀ alkylene chain wherein two or more methylene units are independently replaced by —N(R)— and —SO₂—. In certain embodiments, Q is an optionally substituted C₂₋₁₀ alkylene chain wherein two adjacent methylene units are independently replaced by —O— and —C(O)—. In certain embodiments, Q is an optionally substituted C₂₋₁₀ alkylene chain wherein two adjacent methylene units are independently replaced by —N(R)— and —C(O)—. In certain embodiments, Q is an optionally substituted C₃₋₁₀ alkylene chain wherein two methylene units are independently replaced by two —Cy- groups and one methylene unit is replaced by —O—, —N(R)—, or —S—. In certain embodiments, Q is an optionally substituted C₃₋₁₀ alkylene chain wherein two methylene units are independently replaced by —O—, —N(R)—, or —S— and one methylene unit is replaced by —Cy-.

In some embodiments, Q is an optionally substituted C₂₋₁₀ alkylene chain wherein one or more methylene unit is independently replaced by —Cy-, and wherein one or more —Cy- is independently a bivalent optionally substituted saturated monocyclic ring. In some embodiments, Q is an optionally substituted C₂₋₁₀ alkylene chain wherein two methylene units are independently replaced by —Cy-, and wherein each —Cy- is independently a bivalent optionally substituted saturated monocyclic ring. In some embodiments, one or more —Cy- is independently a bivalent optionally substituted partially unsaturated monocyclic ring. In some embodiments, one or more —Cy- is independently a bivalent optionally substituted aromatic monocyclic ring. In certain embodiments, —Cy- is optionally substituted phenylene.

In some embodiments, one or more —Cy- is independently a bivalent optionally substituted saturated bicyclic ring. In some embodiments, one or more —Cy- is independently a bivalent optionally substituted partially unsaturated bicyclic ring. In some embodiments, one or more —Cy- is independently a bivalent optionally substituted aromatic bicyclic ring. In certain embodiments, —Cy- is optionally substituted naphthylene.

In some embodiments, one or more —Cy- is independently an optionally substituted 6-10 membered arylene. In some embodiments, one or more —Cy- is independently an optionally substituted a 5-10 membered heteroarylene having 1-4 heteroatoms independently selected from oxygen, nitrogen, or sulfur. In some embodiments, one or more —Cy- is independently an optionally substituted a 5-6 membered heteroarylene having 1-4 heteroatoms independently selected from oxygen, nitrogen, or sulfur. In some embodiments, one or more —Cy- is independently an optionally substituted 5 membered heteroarylene having 1-4 heteroatoms independently selected from oxygen, nitrogen, or sulfur. In some embodiments, one or more —Cy- is independently an optionally substituted a 6 membered heteroarylene having 1-4 heteroatoms independently selected from oxygen, nitrogen, or sulfur.

Exemplary optionally substituted —Cy- heteroarylene groups include thienylene, furanylene, pyrrolylene, imidazolylene, pyrazolylene, triazolylene, tetrazolylene, oxazolylene, isoxazolylene, oxadiazolylene, thiazolylene, isothiazolylene, thiadiazolylene, pyridylene, pyridazinylene, pyrimidinylene, pyrazinylene, indolizinylene, purinylene, naphthyridinylene, pteridinylene, indolylene, isoindolylene, benzothienylene, benzofuranylene, dibenzofuranylene, indazolylene, benzimidazolylene, benzthiazolylene, quinolylene, isoquinolylene, cinnolinylene, phthalazinylene, quinazolinylene, quinoxalinylene, 4H-quinolizinylene, carbazolylene, acridinylene, phenazinylene, phenothiazinylene, phenoxazinylene, tetrahydroquinolinylene, tetrahydroisoquinolinylene, pyrido[2,3-b]-1,4-oxazin-3(4H)-onylene, and chromanylene.

In certain embodiments, —Cy- is selected from the group consisting of tetrahydropyranylene, tetrahydrofuranylene, morpholinylene, thiomorpholinylene, piperidinylene, piperazinylene, pyrrolidinylene, tetrahydrothiophenylene, and tetrahydrothiopyranylene, wherein each ring is optionally substituted.

In some embodiments, one or more —Cy- is independently an optionally substituted 3-8 membered carbocyclylene. In some embodiments, one or more —Cy- is independently an optionally substituted 3-6 membered carbocyclylene. In some embodiments, one or more —Cy- is independently an optionally substituted 3-8 membered carbocyclylene. In some embodiments, one or more —Cy- is independently an optionally substituted cyclopropylene, cyclobutylene, cyclopentylene, cyclohexylene, cycloheptylene, or cyclooctylene.

In some embodiments, one or more —Cy- is independently an optionally substituted 3-10 membered heterocyclylene having 1-4 heteroatoms independently selected from oxygen, nitrogen, or sulfur. In some embodiments, one or more —Cy- is independently an optionally substituted 3-8 membered heterocyclylene having 1-4 heteroatoms independently selected from oxygen, nitrogen, or sulfur. In some embodiments, one or more —Cy- is independently an optionally substituted 5-7 membered heterocyclylene having 1-3 heteroatoms independently selected from oxygen, nitrogen, or sulfur. In some embodiments, one or more —Cy- is independently an optionally substituted 3 membered heterocyclylene having 1 heteroatom independently selected from oxygen, nitrogen, or sulfur. In some embodiments, one or more —Cy- is independently an optionally substituted 4 membered heterocyclylene having 1 heteroatom independently selected from oxygen, nitrogen, or sulfur. In some embodiments, one or more —Cy- is independently an optionally substituted 5 membered heterocyclylene having 1-2 heteroatoms independently selected from oxygen, nitrogen, or sulfur. In some embodiments, one or more —Cy- is independently an optionally substituted 6 membered heterocyclylene having 1-3 heteroatoms independently selected from oxygen, nitrogen, or sulfur.

In some embodiments, one or more —Cy- is independently an optionally substituted partially unsaturated 4-10 membered heterocyclylene having 1-4 heteroatoms independently selected from oxygen, nitrogen, or sulfur. In some embodiments, one or more —Cy- is independently an optionally substituted partially unsaturated 5-7 membered heterocyclylene having 1-3 heteroatoms independently selected from oxygen, nitrogen, or sulfur. In some embodiments, one or more —Cy- is independently an optionally substituted partially unsaturated 5 membered heterocyclylene having 1-2 heteroatoms independently selected from oxygen, nitrogen, or sulfur. In some embodiments, one or more —Cy- is independently an optionally substituted partially unsaturated 6 membered heterocyclylene having 1-3 heteroatoms independently selected from oxygen, nitrogen, or sulfur.

Exemplary —Cy- partially unsaturated 5 membered optionally substituted heterocyclylenes include dihydroimidazolylene, dihydrooxazolylene, dihydrothiazolylene, dihydrothiadiazolylene, and dihydrooxadiazolylene.

Exemplary —Cy- saturated 3-8 membered optionally substituted heterocyclenes include oxiranylene, oxetanylene, tetrahydrofuranylene, tetrahydropyranylene, oxepaneylene, aziridineylene, azetidineylene, pyrrolidinylene, piperidinylene, azepanylene, thiiranylene, thietanylene, tetrahydrothiophenylene, tetrahydrothiopyranylene, thiepanylene, dioxolanylene, oxathiolanylene, oxazolidinylene, imidazolidinylene, thiazolidinylene, dithiolanylene, dioxanylene, morpholinylene, oxathianylene, piperazinylene, thiomorpholinylene, dithianylene, dioxepanylene, oxazepanylene, oxathiepanylene, dithiepanylene, diazepanylene, dihydrofuranonylene, tetrahydropyranonylene, oxepanonylene, pyrrolidinonylene, piperidinonylene, azepanonylene, dihydrothiophenonylene, tetrahydrothiopyranonylene, thiepanonylene, oxazolidinonylene, oxazinanonylene, oxazepanonylene, dioxolanonylene, dioxanonylene, dioxepanonylene, oxathiolinonylene, oxathianonylene, oxathiepanonylene, thiazolidinonylene, thiazinanonylene, thiazepanonylene, imidazolidinonylene, tetrahydropyrimidinonylene, diazepanonylene, imidazolidinedionylene, oxazolidinedionylene, thiazolidinedionylene, dioxolanedionylene, oxathiolane dionylene, piperazinedionylene, morpholinedionylene, and thiomorpholinedionylene.

In some embodiments, Q is an optionally substituted C₂₋₁₀ alkylene chain wherein two or three methylene units are independently replaced by —OC(O)NR— and —Cy-. In some embodiments, Q is an optionally substituted C₂₋₁₀ alkylene chain wherein two methylene units are independently replaced by —OC(O)NR— and —Cy-. In some embodiments, Q is an optionally substituted C₂₋₁₀ alkylene chain wherein two methylene units are independently replaced by —OC(O)NR— and —Cy-, and wherein —Cy- is independently an optionally substituted 3-10 membered heterocyclylene having 1-4 heteroatoms independently selected from oxygen, nitrogen, or sulfur. In some embodiments, Q is an optionally substituted C₂₋₁₀ alkylene chain wherein two methylene units are independently replaced by —OC(O)NR— and —Cy-, and wherein —Cy- is independently an optionally substituted 3-4 membered heterocyclylene having 1-4 heteroatoms independently selected from oxygen, nitrogen, or sulfur. In some embodiments, Q is an optionally substituted C₂₋₁₀ alkylene chain wherein two methylene units are independently replaced by —OC(O)NR— and —Cy-, and wherein —Cy- is independently an optionally substituted 4 membered heterocyclylene having 1 heteroatom independently selected from oxygen, nitrogen, or sulfur. In some embodiments, Q is an optionally substituted C₂₋₁₀ alkylene chain wherein two methylene units are independently replaced by —OC(O)NR— and —Cy-, and wherein —Cy- is independently an optionally substituted 4 membered heterocyclylene having 1 heteroatom independently selected from oxygen or nitrogen.

In some embodiments, Q-R¹⁰ is of any of the following formulae:

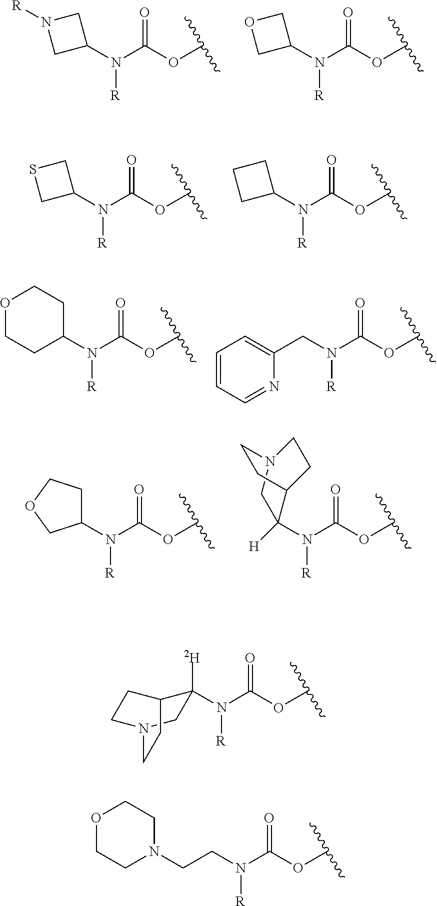

wherein each R is independently as defined above and described herein.

In some embodiments, R¹⁰ is hydrogen and Q is an optionally substituted C₂₋₁₀ alkylene chain wherein two or three methylene units are independently replaced by —OC(O)NR— or —Cy-. Exemplary such Q-R¹⁰ groups are depicted below:

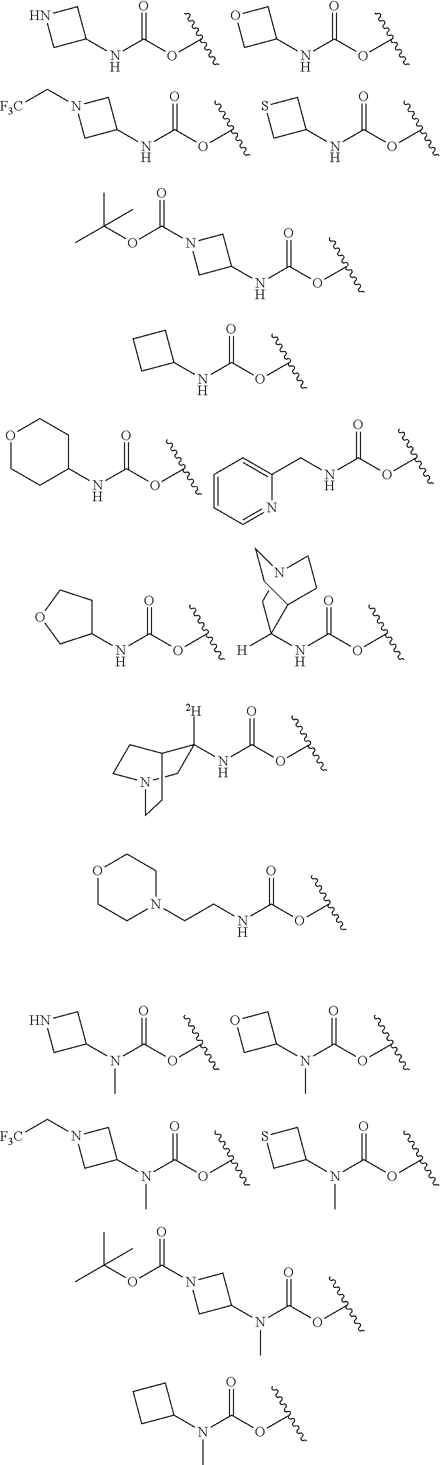

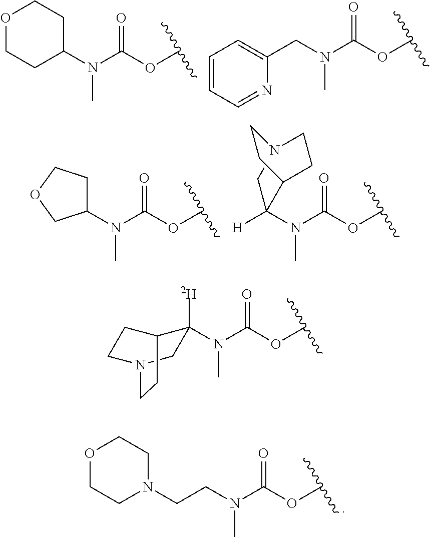

In some embodiments, Q is an optionally substituted C₂₋₁₀ alkylene chain wherein two or three methylene units are independently replaced by —OC(O)— and —Cy-. In some embodiments, Q is an optionally substituted C₂₋₁₀ alkylene chain wherein two methylene units are independently replaced by —OC(O)— and —Cy-. In some embodiments, Q is an optionally substituted C₂₋₁₀ alkylene chain wherein two methylene units are independently replaced by —OC(O)— and —Cy-, wherein —Cy- is independently an optionally substituted 3-10 membered heterocyclylene having 1-4 heteroatoms independently selected from oxygen, nitrogen, or sulfur. In some embodiments, Q is an optionally substituted C₂₋₁₀ alkylene chain wherein two methylene units are independently replaced by —OC(O)— and —Cy-, wherein —Cy- is independently an optionally substituted 4-6 membered heterocyclylene having 1-4 heteroatoms independently selected from oxygen, nitrogen, or sulfur. In some embodiments, Q is an optionally substituted C₂₋₁₀ alkylene chain wherein two methylene units are independently replaced by —OC(O)— and —Cy-, wherein —Cy- is independently an optionally substituted 4-6 membered heterocyclylene having 2 heteroatom independently selected from oxygen, nitrogen, or sulfur. In some embodiments, Q is an optionally substituted C₂₋₁₀ alkylene chain wherein two methylene units are independently replaced by —OC(O)— and —Cy-, wherein —Cy- is independently an optionally substituted 6 membered heterocyclylene having 2 heteroatoms independently selected from oxygen or nitrogen.

In some embodiments, R¹⁰ is hydrogen and Q is an optionally substituted C₂₋₁₀ alkylene chain wherein two or three methylene units are independently replaced by —OC(O)— or —Cy-.

Exemplary Q-R¹⁰ groups are depicted below:

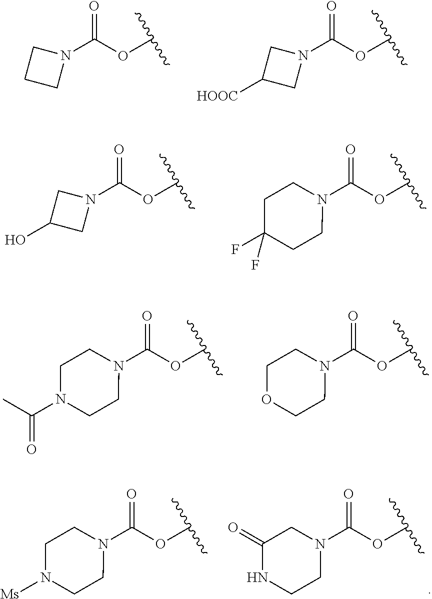

In some embodiments, Q is an optionally substituted C₂₋₁₀ alkylene chain wherein one, two, or three methylene units are independently replaced by —N(R)C(O)—, —N(R)C(O)O—, —N(R)C(O)NR—, or —Cy-.

In some embodiments, Q-R¹⁰ is of any of the following formulae:

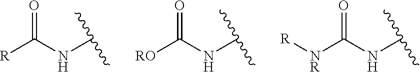

wherein R is as defined above and described herein.

Exemplary Q-R¹⁰ groups are depicted below:

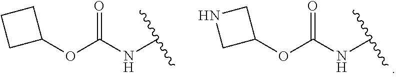 [SMILES: *NC(=O)OC1CCC1 ; *NC(=O)OC1CNC1]

As defined above and herein, R¹⁰ is hydrogen, halogen, an optionally substituted C₁₋₁₀ aliphatic, a suitably protected hydroxyl group, a suitably protected thiol group, a suitably protected amino group, an optionally substituted 3-8 membered saturated, partially unsaturated, or aryl monocyclic ring having 0-4 heteroatoms independently selected from nitrogen, oxygen, or sulfur, an optionally substituted 8-10 membered saturated, partially unsaturated, or aryl bicyclic ring having 0-4 heteroatoms independently selected from nitrogen, oxygen, or sulfur, a detectable moiety, a polymer residue, a peptide, a sugar-containing or sugar-like moiety, or:
wherein when R¹⁰ is a ring, R¹⁰ is optionally substituted at any substitutable carbon with 1-7 R¹¹ and at any substitutable nitrogen with R¹²;
each R¹¹ is independently halogen, R, OR, SR, N(R)₂, N(R)C(O)R, N(R)C(O)OR, N(R)C(O)N(R)₂, N(R)SO₂R, N(R)SO₂OR, S(O)R, SO₂R, OSO₂R, C(O)R, CO₂R, OCO₂R, C(O)N(R)₂, or OC(O)N(R)₂, or wherein:
   two R¹¹ are optionally taken together to form an oxo moiety, an oxime, an optionally substituted hydrazone, an optionally substituted imine, an optionally substituted C₂₋₆ alkylidene, or an optionally substituted 3-8 membered saturated or partially unsaturated fused or spirofused ring having 0-4 heteroatoms independently selected from nitrogen, oxygen, or sulfur; and
each R¹² is independently R, OR, S(O)R, SO₂R, OSO₂R, C(O)R, CO₂R, OCO₂R, C(O)N(R)₂, or OC(O)N(R)₂, an optionally substituted C₁₋₁₀ aliphatic group, a suitably protected amino group, an optionally substituted 3-8 membered saturated, partially unsaturated, or aryl monocyclic ring having 0-4 heteroatoms independently selected from nitrogen, oxygen, or sulfur, an optionally substituted 8-10 membered saturated, partially unsaturated, or aryl bicyclic ring having 0-4 heteroatoms independently selected from nitrogen, oxygen, or sulfur, or wherein:
   R¹² and R¹¹ are optionally taken together to form an optionally substituted 3-8 membered saturated or partially unsaturated fused ring having 0-4 heteroatoms independently selected from nitrogen, oxygen, or sulfur.

In certain embodiments, R¹⁰ is hydrogen. In certain embodiments, R¹⁰ is optionally substituted C₁₋₁₀ aliphatic. In certain embodiments, R¹⁰ is optionally substituted methyl, ethyl, propyl, or butyl. In certain embodiments, R¹⁰ is a suitably protected hydroxyl group, a suitably protected thiol group, or a suitably protected amino group. In certain embodiments wherein Q is a valence bond, R¹⁰ is a suitably protected amino group. In certain embodiments wherein Q is a valence bond, R¹⁰ is an optionally substituted C₁₋₁₀ aliphatic.

In certain embodiments, R¹⁰ is an optionally substituted 3-8 membered saturated monocyclic ring having 1-3 heteroatoms independently selected from nitrogen, oxygen, or sulfur. In certain embodiments, R¹⁰ is an optionally substituted 3-8 membered saturated monocyclic carbocycle. In certain embodiments, R¹⁰ is an optionally substituted 5-6 membered saturated monocyclic ring having 1-2 heteroatoms independently selected from nitrogen, oxygen, or sulfur. In certain embodiments, R¹⁰ is an optionally substituted 5-6 membered saturated monocyclic carbocycle. In certain embodiments, R¹⁰ is an optionally substituted 4 membered saturated monocyclic ring having 1 heteroatom selected from nitrogen, oxygen, or sulfur. In certain embodiments, R¹⁰ is an optionally substituted 4 membered saturated monocyclic carbocycle. In certain embodiments, R¹⁰ is an optionally substituted 7 membered saturated monocyclic ring having 1-2 heteroatoms independently selected from nitrogen, oxygen, or sulfur. In certain embodiments, R¹⁰ is an optionally substituted 7 membered saturated monocyclic carbocycle.

Exemplary R¹⁰ saturated 3-8 membered optionally substituted heterocycles include oxirane, oxetane, tetrahydrofuran, tetrahydropyran, oxepane, aziridine, azetidine, pyrrolidine, piperidine, azepane, thiirane, thietane, tetrahydrothiophene, tetrahydrothiopyran, thiepane, dioxolane, oxathiolane, oxazolidine, imidazolidine, thiazolidine, dithiolane, dioxane, morpholine, oxathiane, piperazine, thiomorpholine, dithiane, dioxepane, oxazepane, oxathiepane, dithiepane, diazepane, dihydrofuranone, tetrahydropyranone, oxepanone, pyrrolidinone, piperidinone, azepanone, dihydrothiophenone, tetrahydrothiopyranone, thiepanone, oxazolidinone, oxazinanone, oxazepanone, dioxolanone, dioxanone, dioxepanone, oxathiolinone, oxathianone, oxathiepanone, thiazolidinone, thiazinanone, thiazepanone, imidazolidinone, tetrahydropyrimidinone, diazepanone, imidazolidinedione, oxazolidinedione, thiazolidinedione, dioxolanedione, oxathiolanedione, piperazinedione, morpholinedione, and thiomorpholinedione.

In some embodiments, R¹⁰ is an optionally substituted oxazepane. In certain embodiments, R¹⁰ is an oxazepane optionally substituted with 1-3 R¹¹ groups and optionally substituted with R¹². In certain embodiments, R¹⁰ is an oxazepane optionally substituted with 1-3 R¹¹ groups and substituted with R¹², wherein one R¹¹ group is taken together with R¹² to form an optionally substituted 3-8 membered saturated or partially unsaturated fused ring having 1-4 heteroatoms independently selected from nitrogen, oxygen, or sulfur. In certain embodiments, the compound is as described above and R¹¹ and R¹² taken together form an optionally substituted 5-6 membered saturated or partially unsaturated fused ring having 1-4 heteroatoms independently selected from nitrogen, oxygen, or sulfur. In certain embodiments, the compound is as described above and R¹¹ and R¹² taken together form an optionally substituted 6 membered saturated fused ring having 1-2 heteroatoms independently selected from nitrogen, oxygen, or sulfur. In certain embodiments, the compound is as described above and R¹¹ and R¹² taken together form an optionally substituted 7 membered saturated fused ring having 1-2 heteroatoms independently selected from nitrogen, oxygen, or sulfur.

In certain embodiments, R¹⁰ is an optionally substituted 3-8 membered partially unsaturated monocyclic ring having 1-3 heteroatoms independently selected from nitrogen, oxygen, or sulfur. In certain embodiments, R¹⁰ is an optionally substituted 3-8 membered partially unsaturated monocyclic carbocycle. In certain embodiments, R¹⁰ is an optionally substituted 5-6 membered partially unsaturated monocyclic ring having 1-2 heteroatoms independently selected from nitrogen, oxygen, or sulfur. In certain embodiments, R¹⁰ is an optionally substituted 5-6 membered partially unsaturated monocyclic carbocycle. In certain embodiments, R¹⁰ is an optionally substituted 5-6 membered aryl ring having 0-3 heteroatoms independently selected from nitrogen, oxygen, or sulfur. In certain embodiments, R¹⁰ is an optionally substituted 5 membered aryl ring having 1-3 heteroatoms independently selected from nitrogen, oxygen, or sulfur. In certain embodiments, R¹⁰ is an optionally substituted 6 membered aryl ring having 1-3 heteroatoms independently selected from nitrogen, oxygen, or sulfur. In certain embodiments, R¹⁰ is an optionally substituted phenyl.

In certain embodiments, R¹⁰ is an optionally substituted 8-10 membered saturated bicyclic ring having 0-4 heteroatoms independently selected from nitrogen, oxygen, or sulfur. In certain embodiments, R¹⁰ is an optionally substituted 8 membered saturated bicyclic ring having 1-3 heteroatoms independently selected from nitrogen, oxygen, or sulfur. In certain embodiments, R¹⁰ is an optionally substituted 8 membered saturated bicyclic carbocycle. In certain embodiments, R¹⁰ is an optionally substituted 9 membered saturated bicyclic ring having 1-3 heteroatoms independently selected from nitrogen, oxygen, or sulfur. In certain embodiments, R¹⁰ is an optionally substituted 9 membered saturated bicyclic carbocycle. In certain embodiments, R¹⁰ is an optionally substituted 10 membered saturated bicyclic ring having 1-3 heteroatoms independently selected from nitrogen, oxygen, or sulfur. In certain embodiments, R¹⁰ is an optionally substituted 10 membered saturated bicyclic carbocycle.

In certain embodiments, R¹⁰ is an optionally substituted 8-10 membered partially unsaturated bicyclic ring having 0-4 heteroatoms independently selected from nitrogen, oxygen, or sulfur. In certain embodiments, R¹⁰ is an optionally substituted 8 membered partially unsaturated bicyclic ring having 1-3 heteroatoms independently selected from nitrogen, oxygen, or sulfur. In certain embodiments, R¹⁰ is an optionally substituted 8 membered partially unsaturated bicyclic carbocycle. In certain embodiments, R¹⁰ is an optionally substituted 9 membered partially unsaturated bicyclic ring having 1-3 heteroatoms independently selected from nitrogen, oxygen, or sulfur. In certain embodiments, R¹⁰ is an optionally substituted 9 membered partially unsaturated bicyclic carbocycle. In certain embodiments, R¹⁰ is an optionally substituted 10 membered partially unsaturated bicyclic ring having 1-3 heteroatoms independently selected from nitrogen, oxygen, or sulfur. In certain embodiments, R¹⁰ is an optionally substituted 10 membered partially unsaturated bicyclic carbocycle.

In certain embodiments, R¹⁰ is an optionally substituted 9-10 membered aryl bicyclic ring having 0-4 heteroatoms independently selected from nitrogen, oxygen, or sulfur. In certain embodiments, R¹⁰ is an optionally substituted 9 membered aryl bicyclic ring having 1-4 heteroatoms independently selected from nitrogen, oxygen, or sulfur. In certain embodiments, R¹⁰ is an optionally substituted 9 membered aryl bicyclic ring having 3 heteroatoms independently selected from nitrogen, oxygen, or sulfur. In certain embodiments, R¹⁰ is an optionally substituted 9 membered aryl bicyclic ring having 2 heteroatoms independently selected from nitrogen, oxygen, or sulfur. In certain embodiments, R¹⁰ is an optionally substituted 9 membered aryl bicyclic ring having 1 heteroatom selected from nitrogen, oxygen, or sulfur. In certain embodiments, R¹⁰ is an optionally substituted 10 membered aryl bicyclic ring having 0-3 heteroatoms independently selected from nitrogen, oxygen, or sulfur. In certain embodiments, R¹⁰ is an optionally substituted 10 membered aryl bicyclic ring having 1-2 heteroatoms independently selected from nitrogen, oxygen, or sulfur. In certain embodiments, R¹⁰ is an optionally substituted naphthyl.

Exemplary optionally substituted R¹⁰ heteroaryl groups include thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, pteridinyl, indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one, or chromanyl.

In certain embodiments, R¹⁰ is a ring, wherein R¹⁰ is optionally substituted at any substitutable carbon with 1-7 R¹¹ and at any substitutable nitrogen with R¹². In certain embodiments, R¹⁰ is a 5-6 membered heterocycle containing 1-2 heteroatoms selected from nitrogen, oxygen, or sulfur and optionally substituted at any substitutable carbon with 1-7 R¹¹ and at any substitutable nitrogen with R¹². In certain embodiments, R¹⁰ is a 5-6 membered heterocycle containing 1-2 heteroatoms selected from nitrogen, oxygen, or sulfur and optionally substituted at any substitutable carbon with 1-5 R¹¹ and at any substitutable nitrogen with R¹². In certain embodiments, R¹⁰ is a 5 membered heterocycle containing 1-2 heteroatoms selected from nitrogen, oxygen, or sulfur and optionally substituted at any substitutable carbon with 1-5 R¹¹ and at any substitutable nitrogen with R¹². In certain embodiments, R¹⁰ is a 6 membered heterocycle containing 1-2 heteroatoms selected from nitrogen, oxygen, or sulfur and optionally substituted at any substitutable carbon with 1-7 R¹¹ and at any substitutable nitrogen with R¹². In certain embodiments, R¹⁰ is a 6 membered heterocycle containing 1-2 heteroatoms selected from nitrogen, oxygen, or sulfur and optionally substituted at any substitutable carbon with 1-5 R¹¹ and at any substitutable nitrogen with R¹². In certain embodiments, R¹⁰ is a 6 membered heterocycle containing one or more nitrogens optionally substituted at any substitutable carbon with 1-5 R¹¹ and at any substitutable nitrogen with R¹². In certain embodiments, R¹⁰ is a 6 membered heterocycle containing one or more oxygens and optionally substituted at any substitutable carbon with 1-5 R¹¹ and at any substitutable nitrogen with R¹².

In certain embodiments, R¹⁰ is selected from the group consisting of tetrahydropyranyl, tetrahydrofuranyl, morpholinyl, thiomorpholinyl, piperidinyl, piperazinyl, pyrrolidinyl, tetrahydrothiophenyl, and tetrahydrothiopyranyl, wherein each ring is optionally substituted at any substitutable carbon with 1-7 R¹¹ and at any substitutable nitrogen with R¹².

In certain embodiments, R¹⁰ is selected from the group consisting of tetrahydropyranyl, morpholinyl, piperidinyl, or piperazinyl, wherein each ring is optionally substituted with 1-7 R¹¹ groups selected from halogen, R, OR, SR, N(R)₂, N(R)C(O)R, N(R)C(O)OR, N(R)C(O)N(R)₂, N(R)SO₂R, N(R)SO₂OR, S(O)R, SO₂R, OSO₂R, C(O)R, CO₂R, OCO₂R, C(O)N(R)₂, or OC(O)N(R)₂, and wherein any substitutable nitrogen is optionally substituted with R¹², wherein R¹² is selected from R, OR, S(O)R, SO₂R, OSO₂R, C(O)R, CO₂R, OCO₂R, C(O)N(R)₂, or OC(O)N(R)₂.

In certain embodiments, R¹⁰ is selected from the group consisting of tetrahydropyranyl, morpholinyl, piperidinyl, piperazinyl, or oxazepanyl, wherein each ring is optionally substituted with 2-3 R¹¹ groups, wherein two R¹¹ are taken together to form an optionally substituted 3-8 membered saturated or partially unsaturated fused or spirofused ring having 0-4 heteroatoms independently selected from nitrogen, oxygen, or sulfur. In certain embodiments, R¹⁰ is as described above, wherein two R¹¹ are taken together to form an optionally substituted 5-6 membered saturated ring having 1-3 heteroatoms. In certain embodiments, R¹⁰ is as described above, wherein two R¹¹ are taken together to form an oxo moiety.

In certain embodiments, R¹⁰ is selected from the group consisting of tetrahydropyranyl, morpholinyl, piperidinyl, piperazinyl, or oxazepanyl, wherein each ring is optionally substituted with at least one R¹¹ group and at least one R¹² group, wherein R¹¹ and R¹² are taken together to form an optionally substituted 3-8 membered saturated or partially unsaturated fused or spirofused ring having 0-4 heteroatoms independently selected from nitrogen, oxygen, or sulfur. In certain embodiments, R¹⁰ is as described above, wherein R¹¹ and R¹² are taken together to form an optionally substituted 5-6 membered saturated ring having 1-3 heteroatoms.

In certain embodiments, R¹⁰ is a detectable moiety. In certain embodiments, R¹⁰ is a polymer residue. In certain embodiments, R¹⁰ is a peptide, a sugar-containing or sugar-like moiety.

Exemplary R¹⁰ groups are depicted below:

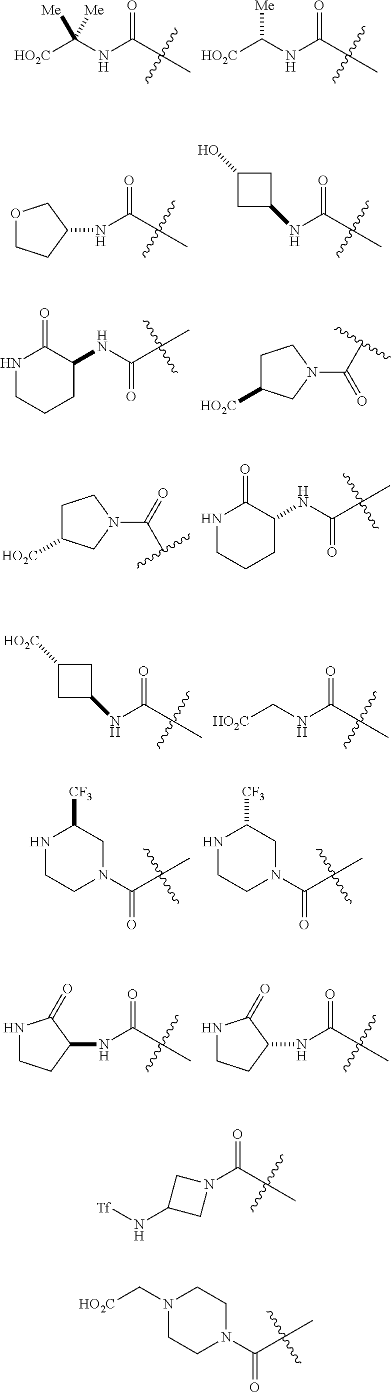

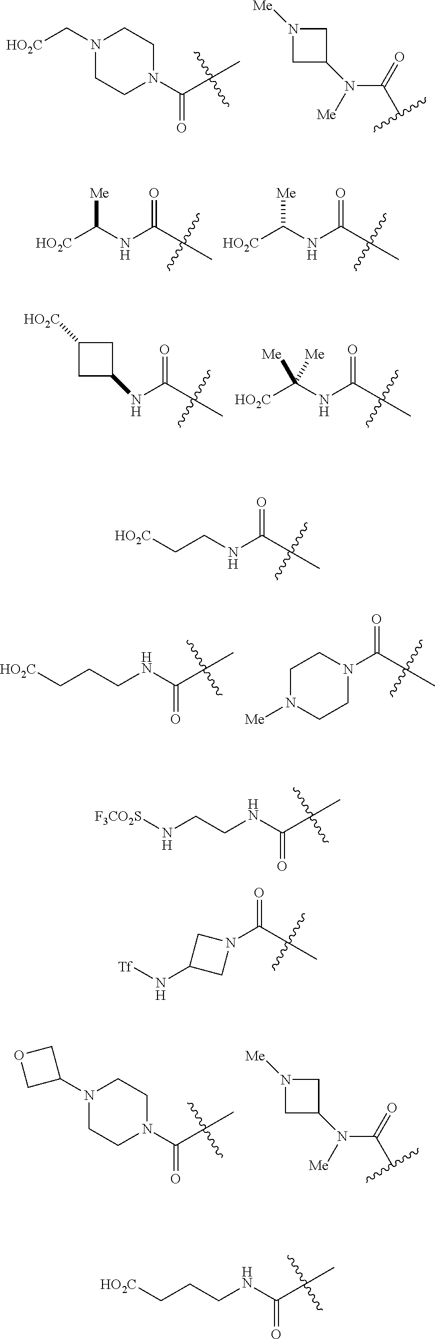

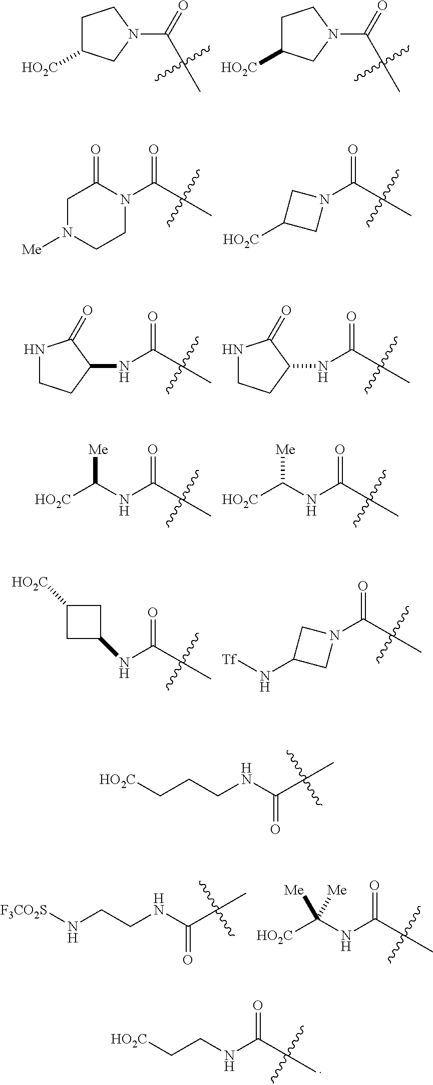

As defined generally above and herein, each R¹¹ is independently halogen, R, OR, SR, N(R)₂, N(R)C(O)R, N(R)C(O)OR, N(R)C(O)N(R)₂, N(R)SO₂R, N(R)SO₂OR, S(O)R, SO₂R, OSO₂R, C(O)R, CO₂R, OCO₂R, C(O)N(R)₂, or OC(O)N(R)₂, or wherein:
two R¹¹ are optionally taken together to form an oxo moiety, an oxime, an optionally substituted hydrazone, an optionally substituted imine, an optionally substituted C₂₋₆ alkylidene, or an optionally substituted 3-8 membered saturated or partially unsaturated fused or spirofused ring having 0-4 heteroatoms independently selected from nitrogen, oxygen, or sulfur.

In some embodiments, one or more R¹¹ is independently halogen, R, OR, SR, or N(R)₂. In some embodiments, one or more R¹¹ is independently halogen. In some embodiments, one or more R¹¹ is independently R. In some embodiments, one or more R¹¹ is independently selected from the group consisting of OR, SR, or N(R)₂. In some embodiments, one or more R¹¹ is independently selected from the group consisting OH, OMe, F, and OCF₃.

In some embodiments, R¹¹ is —C(O)N(R)₂. In certain embodiments, R¹¹ is —C(O)N(R)₂, wherein one or more R is hydrogen. In certain embodiments, R¹¹ is —C(O)N(R)₂, wherein one or more R is optionally substituted C₁₋₆ aliphatic. Exemplary such optionally substituted C₁₋₆ aliphatic groups include optionally substituted alkyl or cycloalkyl groups selected from methyl, ethyl, CF₃, CF₂CF₃, cyclopropyl, cyclopentyl, and cyclohexyl. In certain embodiments, R¹¹ is —C(O)N(R)₂, wherein two R on the same nitrogen atom are optionally taken together with said nitrogen atom to form an optionally substituted 3-8 membered, partially unsaturated, or aryl ring having 1-4 heteroatoms independently selected from nitrogen, oxygen, or sulfur.

In some embodiments, R¹¹ is a C₂₋₆ aliphatic group optionally substituted with a cyclopropyl, cyclobutyl, cyclopentyl, or cyclohexyl moiety. In certain embodiments, R¹¹ is a C₂₋₆ aliphatic group optionally substituted with an oxirane, oxetane, tetrahydrofuran, or tetrahydropyran moiety. In certain embodiments, R¹¹ is a C₂₋₆ aliphatic group optionally substituted with an aziridine, azetidine, pyrrolidine, or piperidine moiety. In certain embodiments, R¹¹ is a C₂₋₆ aliphatic group optionally substituted with an oxazolidine or morpholine moiety. In certain embodiments, R¹¹ is a C₂₋₆ aliphatic group optionally substituted with a dioxolane or dioxane moiety.

In some embodiments, two R¹¹ are taken together to form an oxo moiety or an optionally substituted 3-8 membered saturated or partially unsaturated fused or spirofused ring having 0-4 heteroatoms independently selected from nitrogen, oxygen, or sulfur.

In certain embodiments, two R¹¹ on the same carbon are taken together to form an optionally substituted 3-8 membered saturated spirofused ring having 0-4 heteroatoms independently selected from nitrogen, oxygen, or sulfur. In certain embodiments, two R¹¹ on the same carbon are taken together to form an optionally substituted 3-8 membered saturated spirofused ring having 0-2 heteroatoms independently selected from nitrogen, oxygen, or sulfur. In certain embodiments, two R¹¹ on the same carbon are taken together to form an optionally substituted 5-6 membered saturated spirofused ring having 0-2 heteroatoms independently selected from nitrogen, oxygen, or sulfur.

In certain embodiments, two R¹¹ on the same carbon are taken together to form an optionally substituted 5-8 membered partially unsaturated spirofused ring having 0-4 heteroatoms independently selected from nitrogen, oxygen, or sulfur. In certain embodiments, two R¹¹ on the same carbon are taken together to form an optionally substituted 5-8 membered partially unsaturated spirofused ring having 0-2 heteroatoms independently selected from nitrogen, oxygen, or sulfur. In certain embodiments, two R¹¹ on the same carbon are taken together to form an optionally substituted 5-6 membered partially unsaturated spirofused ring having 0-2 heteroatoms independently selected from nitrogen, oxygen, or sulfur.

In some embodiments, two R¹¹ are taken together to form an optionally substituted 3-8 membered saturated fused ring having 0-4 heteroatoms independently selected from nitrogen, oxygen, or sulfur. In some embodiments, two R¹¹ are taken together to form an optionally substituted 3-8 membered saturated fused ring having 0-2 heteroatoms independently selected from nitrogen, oxygen, or sulfur. In some embodiments, two R¹¹ are taken together to form an optionally substituted 5-6 membered saturated fused ring having 0-2 heteroatoms independently selected from nitrogen, oxygen, or sulfur.

In some embodiments, two R¹¹ are taken together to form an optionally substituted 5-8 membered partially unsaturated fused ring having 0-4 heteroatoms independently selected from nitrogen, oxygen, or sulfur. In some embodiments, two R¹¹ are taken together to form an optionally substituted 5-8 membered partially unsaturated fused ring having 0-2 heteroatoms independently selected from nitrogen, oxygen, or sulfur. In some embodiments, two R¹¹ are taken together to form an optionally substituted 5-6 membered partially unsaturated fused ring having 0-2 heteroatoms independently selected from nitrogen, oxygen, or sulfur.

As defined generally above and herein, each R¹² is independently R, OR, S(O)R, SO₂R, OSO₂R, C(O)R, CO₂R, OCO₂R, C(O)N(R)₂, or OC(O)N(R)₂, an optionally substituted aliphatic group, a suitably protected amino group, an optionally substituted 3-8 membered saturated, partially unsaturated, or aryl monocyclic ring having 0-4 heteroatoms independently selected from nitrogen, oxygen, or sulfur, an optionally substituted 8-10 membered saturated, partially unsaturated, or aryl bicyclic ring having 0-4 heteroatoms independently selected from nitrogen, oxygen, or sulfur, or wherein:
R¹² and R¹¹ are optionally taken together to form an optionally substituted 3-8 membered saturated or partially unsaturated fused ring having 0-4 heteroatoms independently selected from nitrogen, oxygen, or sulfur.

In some embodiments, R¹² and R¹¹ are taken together to form an optionally substituted 3-8 membered saturated or partially unsaturated fused ring having 0-4 heteroatoms independently selected from nitrogen, oxygen, or sulfur.

In some embodiments, R¹² and R¹¹ are taken together to form an optionally substituted 3-8 membered saturated fused ring having 0-4 heteroatoms independently selected from nitrogen, oxygen, or sulfur. In some embodiments, R¹² and R¹¹ are taken together to form an optionally substituted 3-8 membered saturated fused ring having 1-3 heteroatoms independently selected from nitrogen, oxygen, or sulfur. In some embodiments, R¹² and R¹¹ are taken together to form an optionally substituted 5-6 membered saturated fused ring having 0-4 heteroatoms independently selected from nitrogen, oxygen, or sulfur. In some embodiments, R¹² and R¹¹ are taken together to form an optionally substituted 5-6 membered saturated fused ring having 1-3 heteroatoms independently selected from nitrogen, oxygen, or sulfur. In some embodiments, R¹² and R¹¹ are taken together to form an optionally substituted 5 membered saturated fused ring having 0-2 heteroatoms independently selected from nitrogen, oxygen, or sulfur. In some embodiments, R¹² and R¹¹ are taken together to form an optionally substituted 6 membered saturated fused ring having 0-2 heteroatoms independently selected from nitrogen, oxygen, or sulfur.

In some embodiments, R¹² and R¹¹ are taken together to form an optionally substituted 5-8 membered partially unsaturated fused ring having 0-4 heteroatoms independently selected from nitrogen, oxygen, or sulfur. In some embodiments, R¹² and R¹¹ are taken together to form an optionally substituted 5-8 membered partially unsaturated fused ring having 1-3 heteroatoms independently selected from nitrogen, oxygen, or sulfur. In some embodiments, R¹² and R¹¹ are taken together to form an optionally substituted 5-6 membered partially unsaturated fused ring having 0-4 heteroatoms independently selected from nitrogen, oxygen, or sulfur. In some embodiments, R¹² and R¹¹ are taken together to form an optionally substituted 5-6 membered partially unsaturated fused ring having 1-3 heteroatoms independently selected from nitrogen, oxygen, or sulfur. In some embodiments, R¹² and R¹¹ are taken together to form an optionally substituted 5 membered partially unsaturated fused ring having 0-2 heteroatoms independently selected from nitrogen, oxygen, or sulfur. In some embodiments, R¹² and R¹¹ are taken together to form an optionally substituted 6 membered partially unsaturated fused ring having 0-2 heteroatoms independently selected from nitrogen, oxygen, or sulfur.

In some embodiments, R¹² is —C(O)N(R)₂. In certain embodiments, R¹² is —C(O)N(R)₂, wherein one or more R is hydrogen. In certain embodiments, R¹² is —C(O)N(R)₂, wherein one or more R is optionally substituted C₁₋₆ aliphatic. Exemplary such optionally substituted C₁₋₆ aliphatic groups include optionally substituted alkyl or cycloalkyl groups selected from methyl, ethyl, CF₃, CF₂CF₃, cyclopropyl, cyclopentyl, and cyclohexyl. In certain embodiments, R¹² is —C(O)N(R)₂, wherein two R on the same nitrogen atom are optionally taken together with said nitrogen atom to form an optionally substituted 3-8 membered, partially unsaturated, or aryl ring having 1-4 heteroatoms independently selected from nitrogen, oxygen, or sulfur.

In some embodiments, R¹² is an optionally substituted aliphatic group. In some embodiments, R¹² is an optionally substituted C₁₋₁₉ aliphatic group. In some embodiments, R¹² is an optionally substituted C₁₋₁₈ aliphatic group. In some embodiments, R¹² is an optionally substituted C₁₋₁₇ aliphatic group. In some embodiments, R¹² is an optionally substituted C₁₋₁₆ aliphatic group. In some embodiments, R¹² is an optionally substituted C₁₋₁₅ aliphatic group. In some embodiments, R¹² is an optionally substituted C₁₋₁₄ aliphatic group. In some embodiments, R¹² is an optionally substituted C₁₋₁₃ aliphatic group. In some embodiments, R¹² is an optionally substituted C₁₋₁₂ aliphatic group. In some embodiments, R¹² is an optionally substituted C₁₋₁₁ aliphatic group. In some embodiments, R¹² is an optionally substituted C₁₋₁₀ aliphatic group. In some embodiments, R¹² is an optionally substituted C₁₋₉ aliphatic group. In some embodiments, R¹² is an optionally substituted C₁₋₈ aliphatic group. In some embodiments, R¹² is an optionally substituted C₁₋₇ aliphatic group. In some embodiments, R¹² is an optionally substituted C₁₋₆ aliphatic group. In some embodiments, R¹² is an optionally substituted C₆ aliphatic group. In some embodiments, R¹² is an optionally substituted C₅ aliphatic group. In some embodiments, R¹² is an optionally substituted C₄ aliphatic group. In some embodiments, R¹² is an optionally substituted C₃ aliphatic group. In some embodiments, R¹² is an optionally substituted C₂ aliphatic group. In some embodiments, R¹² is an optionally substituted C₁ aliphatic group.

In some embodiments, R¹² is a C₂₋₆ aliphatic group optionally substituted with a cyclopropyl, cyclobutyl, cyclopentyl, or cyclohexyl moiety. In certain embodiments, R¹² is a C₂₋₆ aliphatic group optionally substituted with an oxirane, oxetane, tetrahydrofuran, or tetrahydropyran moiety. In certain embodiments, R¹² is a C₂₋₆ aliphatic group optionally substituted with an aziridine, azetidine, oxetane, oxirane, pyrrolidine, or piperidine moiety. In certain embodiments, R¹² is a C₂₋₆ aliphatic group optionally substituted with a cyclopropyl or cyclobutyl moiety. In certain embodiments, R¹² is a C₂₋₆ aliphatic group optionally substituted with a dioxolane or dioxane moiety.

In certain embodiments, R¹² is an optionally substituted 3-8 membered saturated monocyclic ring having 1-3 heteroatoms independently selected from nitrogen, oxygen, or sulfur. In certain embodiments, R¹² is an optionally substituted 3-8 membered saturated monocyclic carbocycle. In certain embodiments, R¹² is an optionally substituted 5-6 membered saturated monocyclic ring having 1-2 heteroatoms independently selected from nitrogen, oxygen, or sulfur. In certain embodiments, R¹² is an optionally substituted 5-6 membered saturated monocyclic carbocycle. In certain embodiments, R¹² is an optionally substituted 7 membered saturated monocyclic ring having 1-2 heteroatoms independently selected from nitrogen, oxygen, or sulfur. In certain embodiments, R¹² is an optionally substituted 7 membered saturated monocyclic carbocycle.

Exemplary R¹² saturated 3-8 membered optionally substituted heterocycles include oxirane, oxetane, tetrahydrofuran, tetrahydropyran, oxepane, aziridine, azetidine, pyrrolidine, piperidine, azepane, thiirane, thietane, tetrahydrothiophene, tetrahydrothiopyran, thiepane, dioxolane, oxathiolane, oxazolidine, imidazolidine, thiazolidine, dithiolane, dioxane, morpholine, oxathiane, piperazine, thiomorpholine, dithiane, dioxepane, oxazepane, oxathiepane, dithiepane, diazepane, dihydrofuranone, tetrahydropyranone, oxepanone, pyrrolidinone, piperidinone, azepanone, dihydrothiophenone, tetrahydrothiopyranone, thiepanone, oxazolidinone, oxazinanone, oxazepanone, dioxolanone, dioxanone, dioxepanone, oxathiolinone, oxathianone, oxathiepanone, thiazolidinone, thiazinanone, thiazepanone, imidazolidinone, tetrahydropyrimidinone, diazepanone, imidazolidinedione, oxazolidinedione, thiazolidinedione, dioxolanedione, oxathiolanedione, piperazinedione, morpholinedione, and thiomorpholinedione.

In certain embodiments, R¹² is an optionally substituted 3-8 membered partially unsaturated monocyclic ring having 1-3 heteroatoms independently selected from nitrogen, oxygen, or sulfur. In certain embodiments, R¹² is an optionally substituted 3-8 membered partially unsaturated monocyclic carbocycle. In certain embodiments, R¹² is an optionally substituted 5-6 membered partially unsaturated monocyclic ring having 1-2 heteroatoms independently selected from nitrogen, oxygen, or sulfur. In certain embodiments, R¹² is an optionally substituted 5-6 membered partially unsaturated monocyclic carbocycle. In certain embodiments, R¹² is an optionally substituted 5-6 membered aryl ring having 0-3 heteroatoms independently selected from nitrogen, oxygen, or sulfur. In certain embodiments, R¹² is an optionally substituted 5 membered aryl ring having 1-3 heteroatoms independently selected from nitrogen, oxygen, or sulfur. In certain embodiments, R¹² is an optionally substituted 6 membered aryl ring having 1-3 heteroatoms independently selected from nitrogen, oxygen, or sulfur. In certain embodiments, R¹² is an optionally substituted phenyl.

In certain embodiments, R¹² is an optionally substituted 8-10 membered saturated bicyclic ring having 0-4 heteroatoms independently selected from nitrogen, oxygen, or sulfur. In certain embodiments, R¹² is an optionally substituted 8 membered saturated bicyclic ring having 1-3 heteroatoms independently selected from nitrogen, oxygen, or sulfur. In certain embodiments, R¹² is an optionally substituted 8 membered saturated bicyclic carbocycle. In certain embodiments, R¹² is an optionally substituted 9 membered saturated bicyclic ring having 1-3 heteroatoms independently selected from nitrogen, oxygen, or sulfur. In certain embodiments, R¹² is an optionally substituted 9 membered saturated bicyclic carbocycle. In certain embodiments, R¹² is an optionally substituted 10 membered saturated bicyclic ring having 1-3 heteroatoms independently selected from nitrogen, oxygen, or sulfur. In certain embodiments, R¹² is an optionally substituted 10 membered saturated bicyclic carbocycle.

In certain embodiments, R¹² is an optionally substituted 8-10 membered partially unsaturated bicyclic ring having 0-4 heteroatoms independently selected from nitrogen, oxygen, or sulfur. In certain embodiments, R¹² is an optionally substituted 8 membered partially unsaturated bicyclic ring having 1-3 heteroatoms independently selected from nitrogen, oxygen, or sulfur. In certain embodiments, R¹² is an optionally substituted 8 membered partially unsaturated bicyclic carbocycle. In certain embodiments, R¹² is an optionally substituted 9 membered partially unsaturated bicyclic ring having 1-3 heteroatoms independently selected from nitrogen, oxygen, or sulfur. In certain embodiments, R¹² is an optionally substituted 9 membered partially unsaturated bicyclic carbocycle. In certain embodiments, R¹² is an optionally substituted 10 membered partially unsaturated bicyclic ring having 1-3 heteroatoms independently selected from nitrogen, oxygen, or sulfur. In certain embodiments, R¹² is an optionally substituted 10 membered partially unsaturated bicyclic carbocycle.

In certain embodiments, R¹² is an optionally substituted 9-10 membered aryl bicyclic ring having 0-4 heteroatoms independently selected from nitrogen, oxygen, or sulfur. In certain embodiments, R¹² is an optionally substituted 9 membered aryl bicyclic ring having 1-4 heteroatoms independently selected from nitrogen, oxygen, or sulfur. In certain embodiments, R¹² is an optionally substituted 9 membered aryl bicyclic ring having 3 heteroatoms independently selected from nitrogen, oxygen, or sulfur. In certain embodiments, R¹² is an optionally substituted 9 membered aryl bicyclic ring having 2 heteroatoms independently selected from nitrogen, oxygen, or sulfur. In certain embodiments, R¹² is an optionally substituted 9 membered aryl bicyclic ring having 1 heteroatom selected from nitrogen, oxygen, or sulfur. In certain embodiments, R¹² is an optionally substituted 10 membered aryl bicyclic ring having 0-3 heteroatoms independently selected from nitrogen, oxygen, or sulfur. In certain embodiments, R¹² is an optionally substituted 10 membered aryl bicyclic ring having 1-2 heteroatoms independently selected from nitrogen, oxygen, or sulfur. In certain embodiments, R¹² is an optionally substituted naphthyl.

Exemplary optionally substituted R¹² heteroaryl groups include thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, pteridinyl, indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one, or chromanyl.

Exemplary R¹² groups are depicted below:

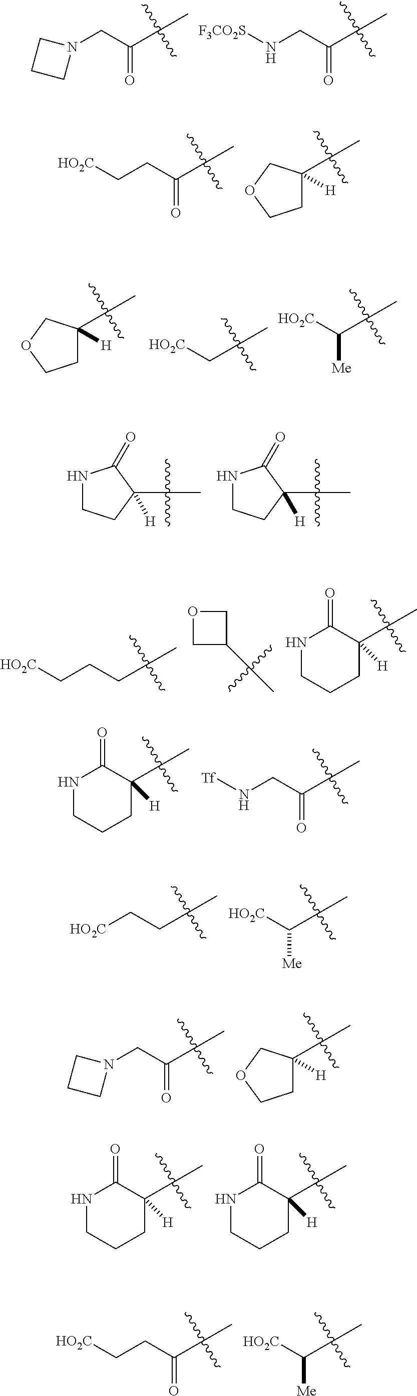

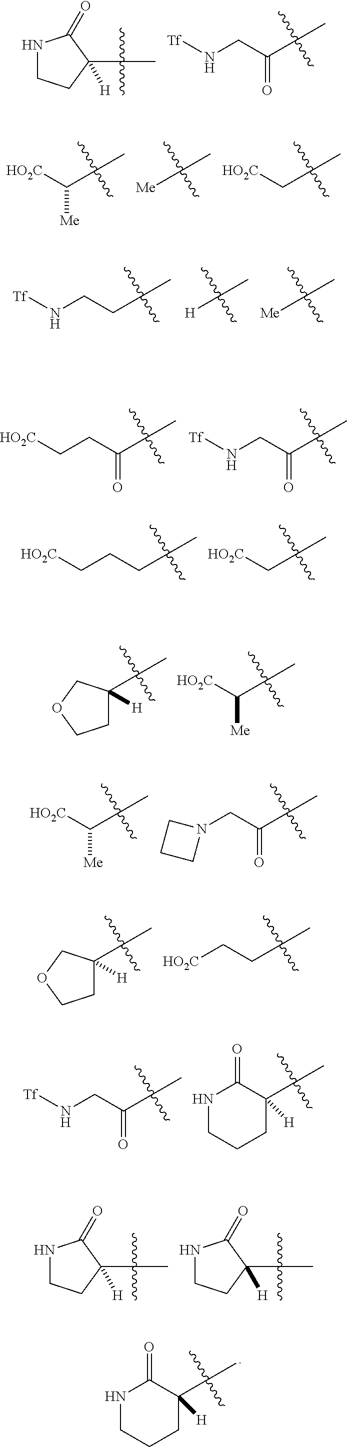

In some embodiments, the present invention provides a compound of the formula V-a:

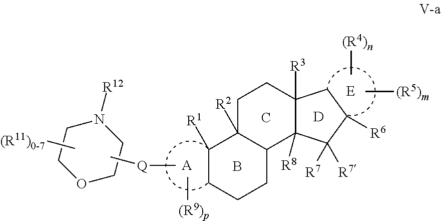

or a pharmaceutically acceptable salt thereof, wherein each variable is defined above and in classes and subclasses herein.

In some embodiments, the present invention provides a compound of either of the formulae V-a(1) or V-a(2):

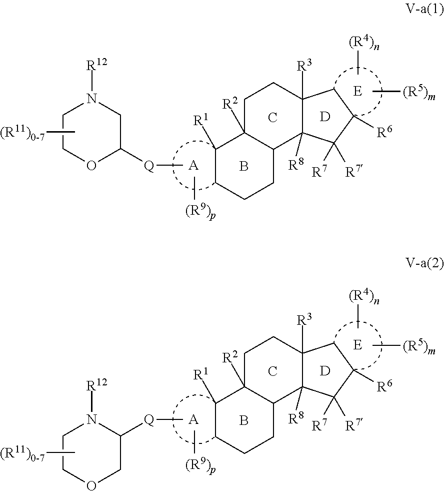

or a pharmaceutically acceptable salt thereof, wherein each variable is defined above and in classes and subclasses herein.

In some embodiments, the present invention provides a compound of either of the formulae V-a(1)a or V-a(1)b:

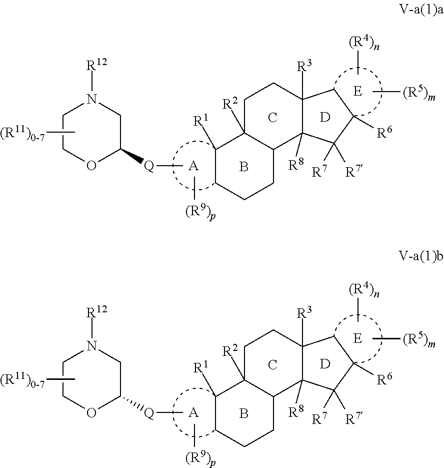

or a pharmaceutically acceptable salt thereof, wherein each variable is defined above and in classes and subclasses herein.

In some embodiments, the present invention provides a compound of either of the formulae V-a(2)a or V-a(2)b:

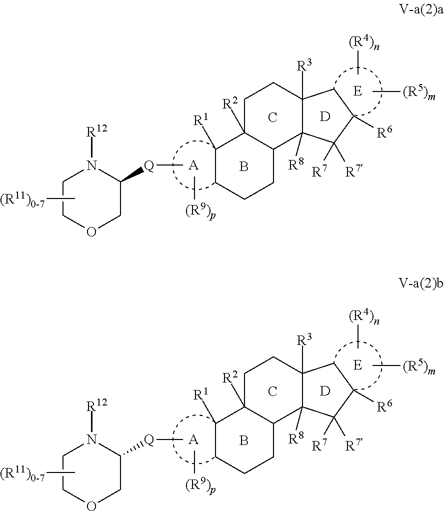

or a pharmaceutically acceptable salt thereof, wherein each variable is defined above and in classes and subclasses herein.

In some embodiments, R¹⁰ is of the following formula:

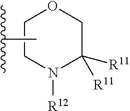

wherein each R¹¹ and R¹² are as defined above and described herein. In certain embodiments, R¹⁰ is of the formula shown above wherein one or more R¹¹ is R. In certain embodiments, R¹⁰ is of the formula shown above wherein R¹¹ are taken together to form an oxo moiety. In certain embodiments, R¹⁰ is of the formula shown above wherein R¹¹ are taken together to form an optionally substituted 3-8 membered saturated or partially unsaturated fused or spirofused ring having 0-4 heteroatoms independently selected from nitrogen, oxygen, or sulfur. In certain embodiments, R¹⁰ is of the formula shown above wherein R¹¹ and R¹² are taken together to form an optionally substituted 3-8 membered saturated or partially unsaturated fused ring having 0-4 heteroatoms independently selected from nitrogen, oxygen, or sulfur.

In some embodiments, R¹⁰ is of the following formula:

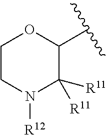

wherein each R¹¹ and R¹² are as defined above and described herein.

In some embodiments, R¹⁰ is of either of the following formulae:

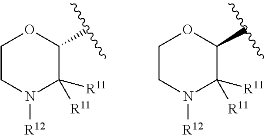

wherein each R¹¹ and R¹² are as defined above and described herein.

In some embodiments, R¹⁰ is of either of the following formulae:

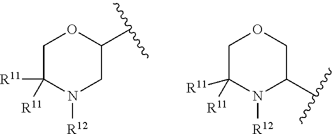

wherein each R¹¹ and R¹² are as defined above and described herein. In certain embodiments, R¹⁰ is of the formula shown above wherein one or more R¹¹ is R. In certain embodiments, R¹⁰ is of the formula shown above wherein R¹¹ are taken together to form an oxo moiety. In certain embodiments, R¹⁰ is of the formula shown above wherein R¹¹ are taken together to form an optionally substituted 3-8 membered saturated or partially unsaturated spirofused ring having 0-4 heteroatoms independently selected from nitrogen, oxygen, or sulfur.

In some embodiments, R¹⁰ is of any one of the following formulae:

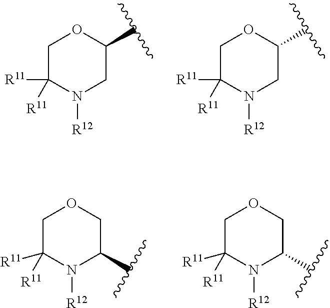

wherein each R¹¹ and R¹² are as defined above and described herein.

In some embodiments, R¹⁰ is of either of the following formulae:

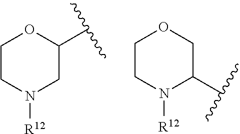

wherein R¹² is as defined above and described herein. In some embodiments, R¹⁰ is of the formula shown above, wherein R¹² is an optionally substituted aliphatic group as described and defined generally above and herein. In certain embodiments, R¹⁰ is as depicted above and R¹² is an optionally substituted aliphatic group wherein one, two, three, or four carbon atoms are independently substituted with a suitable monovalent substituent as defined and described herein. In certain embodiments, R¹⁰ is as depicted above and R¹² is an optionally substituted aliphatic group wherein one, two, three, or four carbon atoms are independently substituted with a suitable divalent substituent as defined and described herein. In certain embodiments, R¹⁰ is as depicted above and R¹² is an optionally substituted aliphatic group wherein one, two, three, or four carbon atoms are independently substituted with a suitable monovalent substituent as defined and described herein and wherein one of the one, two, three, or four carbon atoms is further substituted with a suitable divalent substituent as defined and described herein.

In certain embodiments, R¹⁰ is of the formula shown above, wherein R¹² is an optionally substituted aliphatic group wherein one or two carbon atoms are independently substituted with a suitable monovalent substituent and wherein one or two carbon atoms are independently substituted with a suitable divalent substituent.

In certain embodiments, R¹⁰ is of the formula shown above, wherein R¹² is of any of the following formulae:

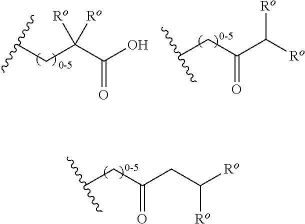

wherein R^{o} is as defined and described generally above and herein. In certain embodiments, each R^{o} is independently hydrogen, C₁₋₆ aliphatic, or a 5-6-membered saturated, partially unsaturated, or an aryl ring having 0-4 heteroatoms independently selected from nitrogen, oxygen, or sulfur. In certain embodiments, R¹² is of one of the formula depicted above, wherein two independent occurrences of R^{o} taken together form an optionally substituted 3-12-membered saturated, partially unsaturated, or aryl mono- or bicyclic ring having 0-4 heteroatoms independently selected from nitrogen, oxygen, or sulfur. In certain embodiments, R¹² is of any one of the formulae depicted above, wherein two independent occurrences of R^{o} taken together form an optionally substituted 3-8-membered saturated ring having 0-4 heteroatoms independently selected from nitrogen, oxygen, or sulfur. In certain embodiments, R¹² is of any one of the formulae depicted above, wherein two independent occurrences of R^{o} taken together form an optionally substituted 3-membered saturated ring having 0-1 heteroatom selected from nitrogen, oxygen, or sulfur. In certain embodiments, R¹² is of any one of the formulae depicted above, wherein two independent occurrences of R^{o} taken together form an optionally substituted cyclopropyl, cyclobutyl, cyclopentyl, or cyclohexyl ring. In certain embodiments, R¹² is of any one of the formulae depicted above, wherein two independent occurrences of R^{o} taken together form an optionally substituted 3-membered saturated ring having 1 heteroatom selected from nitrogen, oxygen, or sulfur. In certain embodiments, R¹² is of any one of the formulae depicted above, wherein two independent occurrences of R^{o} taken together form an optionally substituted 4-membered saturated ring having 1 heteroatom selected from nitrogen, oxygen, or sulfur. In certain embodiments, R¹² is of any one of the formulae depicted above, wherein two independent occurrences of R^{o} taken together form an optionally substituted 5-membered saturated ring having 1 heteroatom selected from nitrogen, oxygen, or sulfur. In certain embodiments, R¹² is of any one of the formulae depicted above, wherein two independent occurrences of R^{o} taken together form an optionally substituted 6-membered saturated ring having 1 heteroatom selected from nitrogen, oxygen, or sulfur.

Exemplary R¹² groups are depicted below:

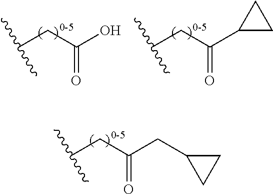

In some embodiments, R¹⁰ is of any of the following formulae:

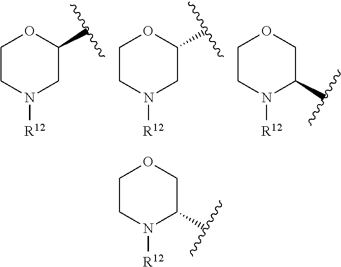

wherein R¹² is as defined above and described herein. In some embodiments, R¹⁰ is of any one of the formulae shown above, wherein R¹² is an optionally substituted 5-6 membered saturated, partially unsaturated, or aryl monocyclic ring having 0-2 heteroatoms independently selected from nitrogen, oxygen, or sulfur. In some embodiments, R¹⁰ is of any one of the formulae shown above, wherein R¹² is an optionally substituted 8-10 membered saturated, partially unsaturated, or aryl bicyclic ring having 0-4 heteroatoms independently selected from nitrogen, oxygen, or sulfur.

In some embodiments, R¹⁰ is of the following formula:

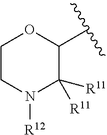

wherein R¹¹ and R¹² are as defined above and described herein. In certain embodiments, R¹⁰ is of the formula shown above, wherein R¹² is hydrogen. In certain embodiments, R¹⁰ is of the formula shown above, wherein R¹² is an optionally substituted C₁₋₂₀ aliphatic group. In certain embodiments, R¹⁰ is of the formula shown above, wherein R¹² is an optionally substituted C₁₋₆ aliphatic group. Exemplary such optionally substituted C₁₋₆ aliphatic groups include cycloalkyl groups such as cyclopropyl, cyclopentyl, and cyclohexyl groups. In certain other embodiments, R¹⁰ is of the formula shown above, wherein R¹² is an optionally substituted 3-8 membered saturated, partially unsaturated, or aryl ring having 0-4 heteroatoms independently selected from nitrogen, oxygen, or sulfur.

In some embodiments, R¹⁰ is of either of the following formulae:

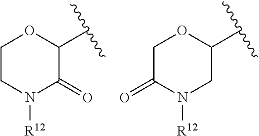

wherein R¹² is as defined above and described herein.

In some embodiments, R¹⁰ is of any one of the following formulae:

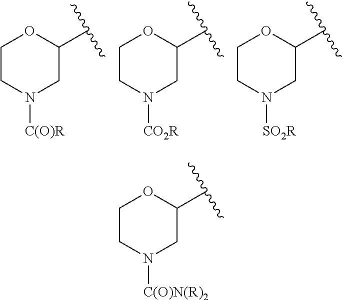

wherein each R is as defined above and described herein, and wherein R is not hydrogen when R¹⁰ is

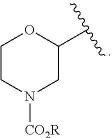

In some embodiments, R¹⁰ is of any one of the following formulae:

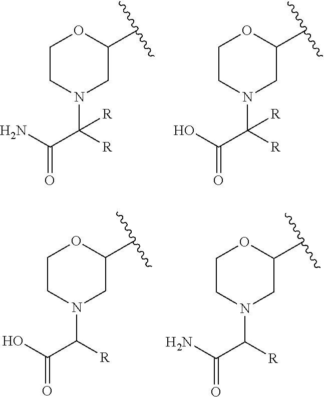

wherein each R is as defined above and described herein.

In some embodiments, R¹⁰ is of the following formula:

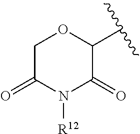

wherein R¹² is as defined above and described herein. In some embodiments, R¹⁰ is of the formula shown above wherein R¹² is an optionally substituted C₁₋₆ aliphatic group. In certain embodiments, R¹² is an optionally substituted C₂ aliphatic group.

In some embodiments, R¹⁰ is of the following formula:

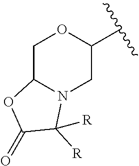

wherein each R is as defined above and described herein.

In some embodiments, R¹⁰ is of either of the following formula:

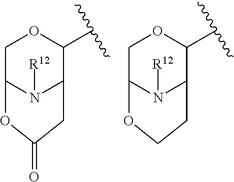

wherein each R¹² is as defined above and described herein.

In some embodiments, R¹⁰ is of either of the following formula:

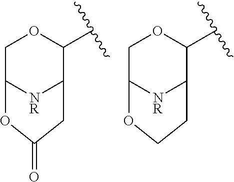

wherein each R is as defined above and described herein.

In some embodiments, R¹⁰ is of any one of the following formulae:

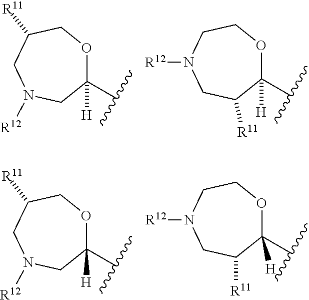

wherein R¹¹ and R¹² are as defined above and described herein.

In some embodiments, R¹⁰ is of any one of the following formulae:

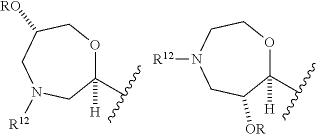

wherein R¹¹ and R¹² are as defined above and described herein.

In some embodiments, R¹⁰ is of any one of the following formulae:

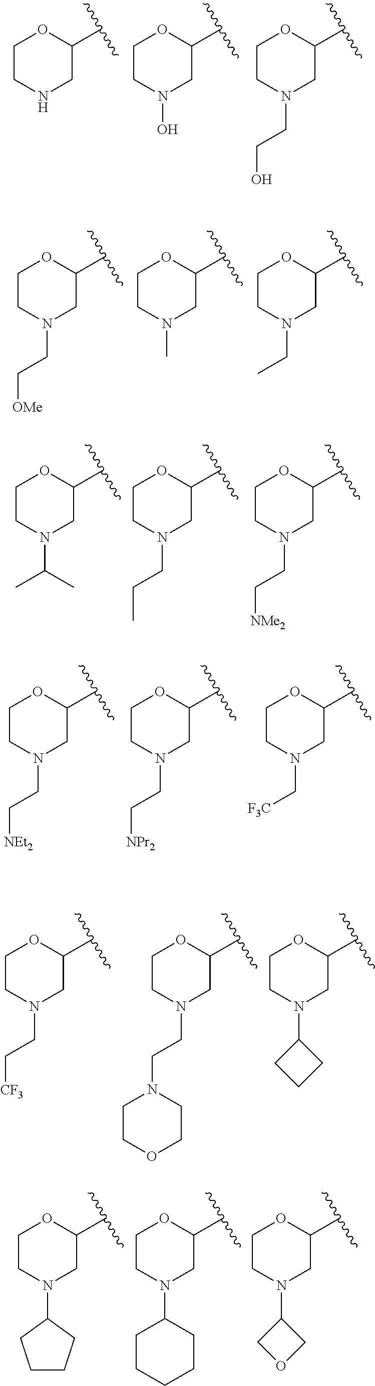

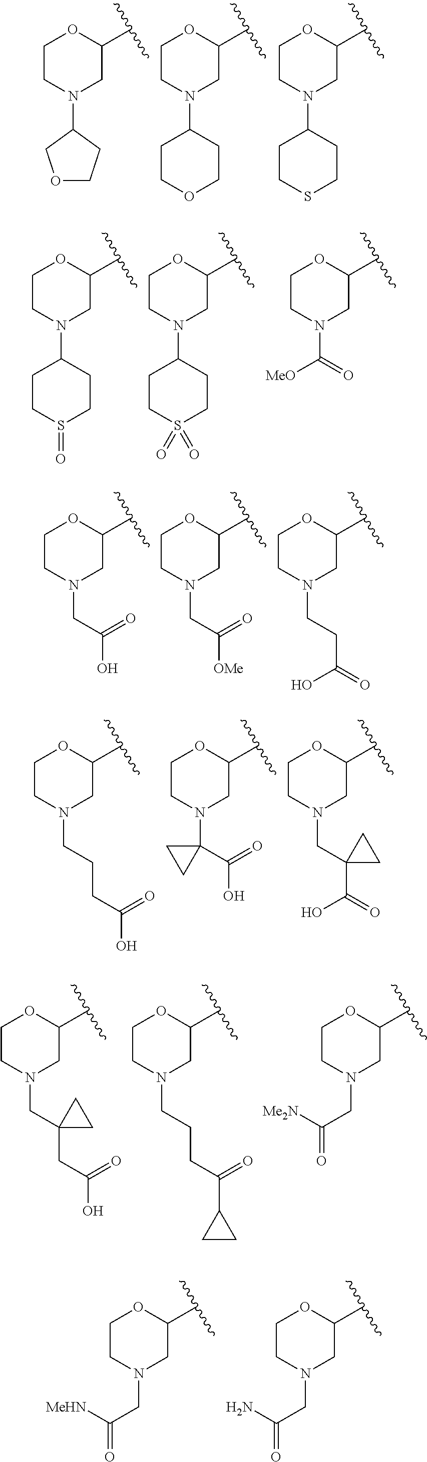

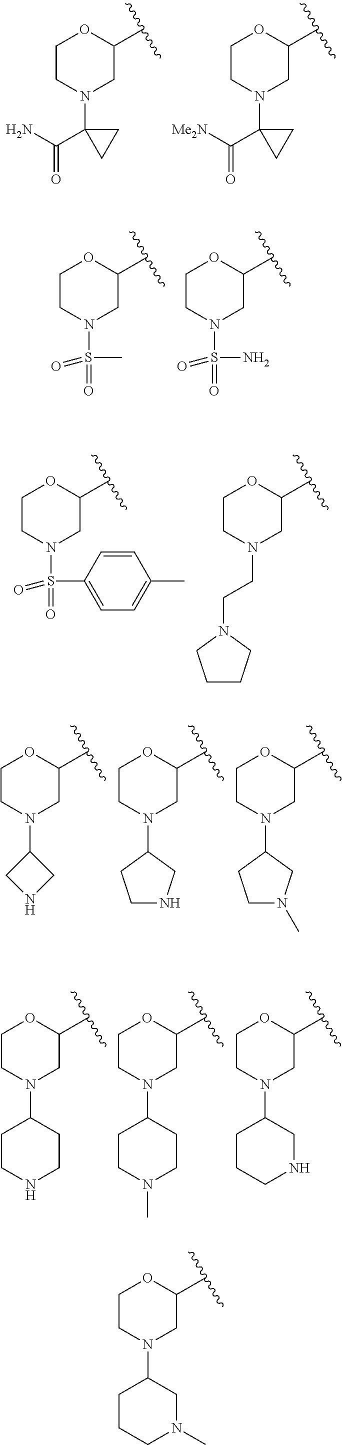

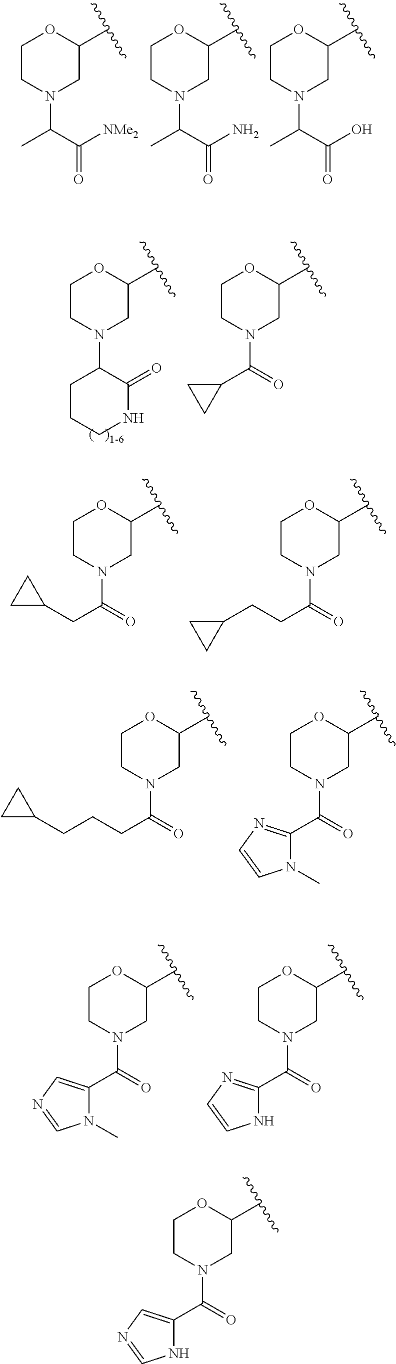

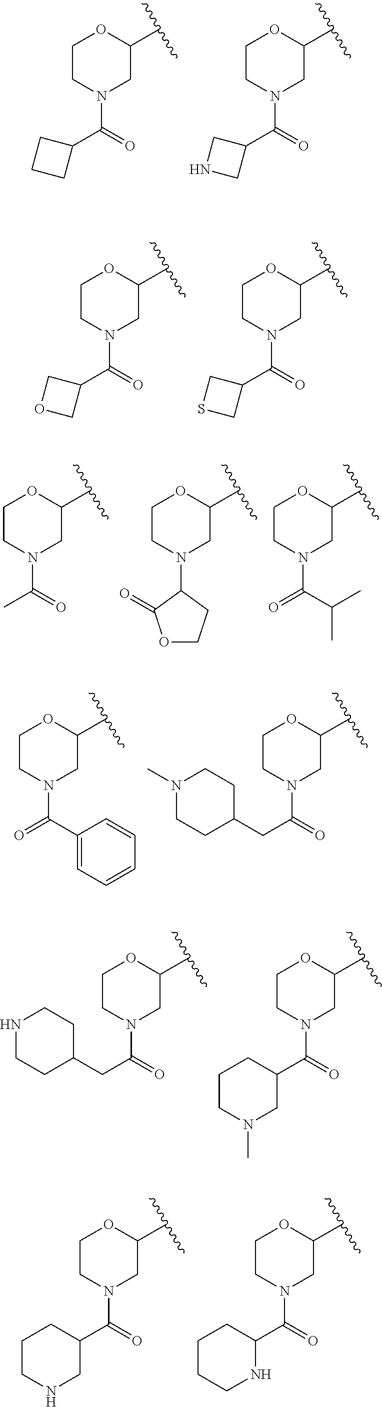

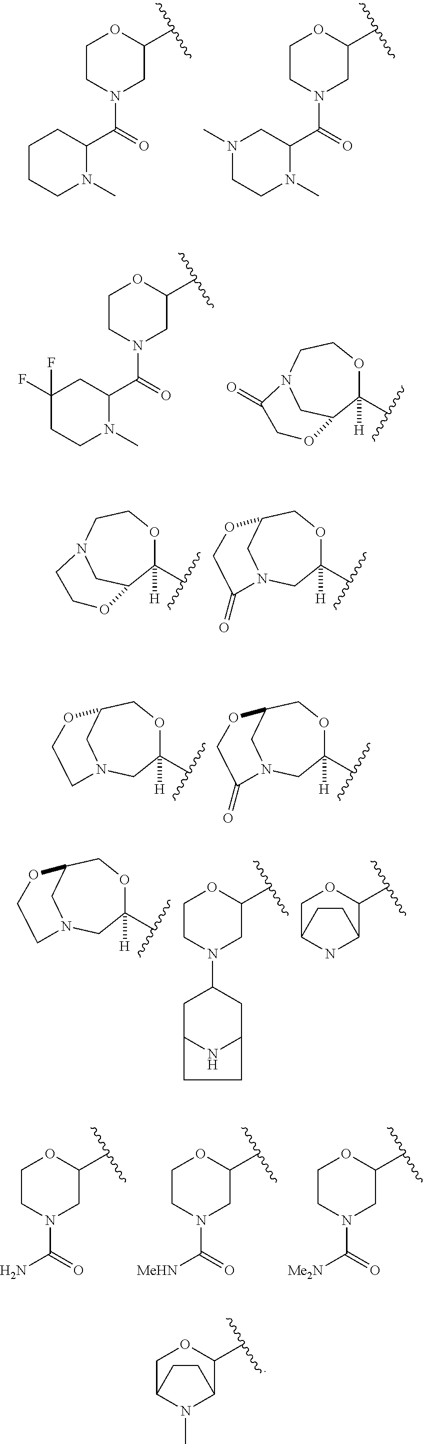

One of skill in the art would appreciate that the present invention contemplates any possible stereoisomeric forms of the above-depicted R¹⁰ groups. Exemplary such possible chiral centers are as shown below:

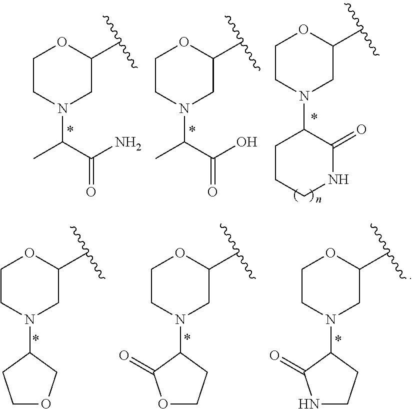

In certain embodiments, R¹⁰ is of either of the following formulae:

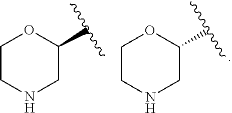

In certain embodiments, R¹⁰ is of either of the following formulae:

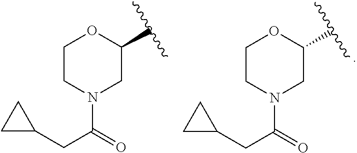 [SMILES: *[C@H]1CN(C(=O)CC2CC2)CCO1 ; *[C@@H]1CN(C(=O)CC2CC2)CCO1]

In certain embodiments, R¹⁰ is of either of the following formulae:

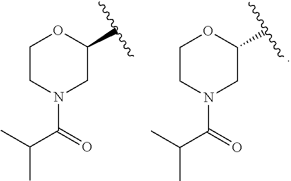 [SMILES: *[C@H]1CN(C(=O)C(C)C)CCO1 ; *[C@@H]1CN(C(=O)C(C)C)CCO1]

In certain embodiments, R¹⁰ is of either of the following formulae:

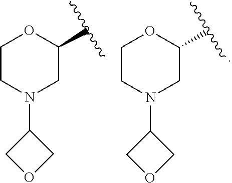

In some embodiments, R¹⁰ is of either of the following formulae:

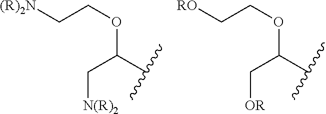

wherein each R is as defined above and described herein.

In some embodiments, R¹⁰ is of the following formula:

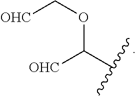 [SMILES: *C(C=O)OCC=O]

In some embodiments, R¹⁰ is of the following formula:

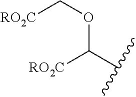

wherein each R is as defined above and described herein.

In some embodiments, R¹⁰ is of the following formula:

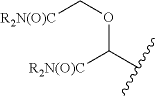

wherein each R is as defined above and described herein.

n some embodiments, R¹⁰ is of the following formula:

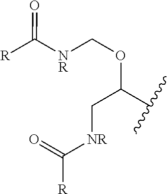

wherein each R is as defined above and described herein.

In some embodiments, R¹⁰ is of any of the following formulae:

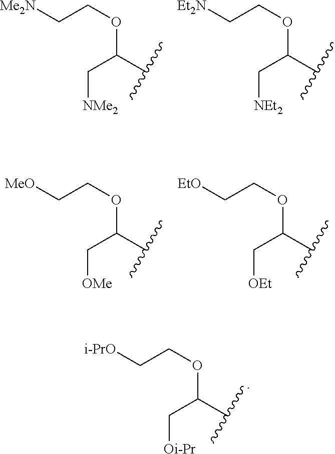

In some embodiments, the present invention provides a compound of the formula V-a-i:

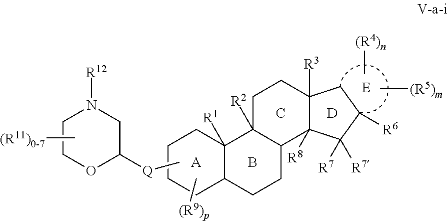

or a pharmaceutically acceptable salt thereof, wherein each variable is defined above and in classes and subclasses herein.

In some embodiments, the present invention provides a compound of either of the formulae V-a-ii or V-a-iii:

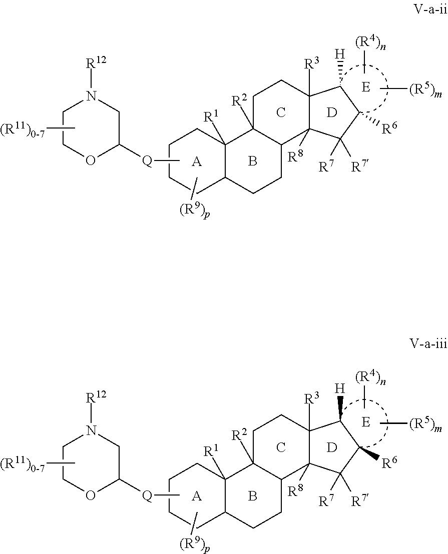

or a pharmaceutically acceptable salt thereof, wherein each variable is defined above and in classes and subclasses herein.

In some embodiments, the present invention provides a compound of either of the formulae V-a-iv or V-a-v:

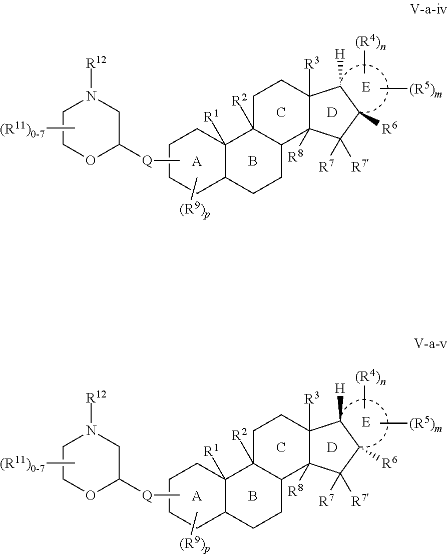

or a pharmaceutically acceptable salt thereof, wherein each variable is defined above and in classes and subclasses herein.

In some embodiments, the present invention provides a compound of the formula V-a-vi:

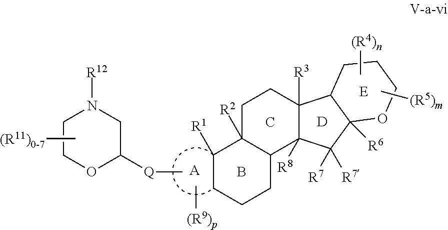

or a pharmaceutically acceptable salt thereof, wherein each variable is defined above and in classes and subclasses herein.

In some embodiments, the present invention provides a compound of either of the formulae V-a-vii or V-a-viii:

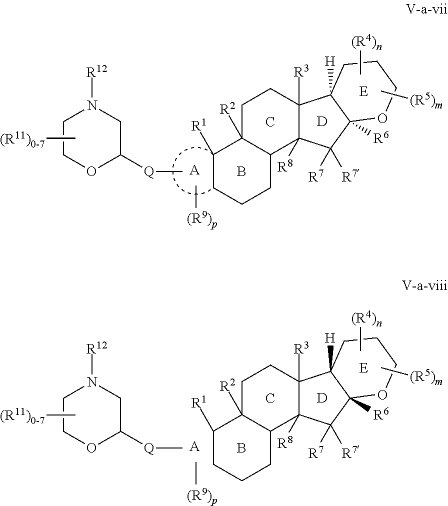

or a pharmaceutically acceptable salt thereof, wherein each variable is defined above and in classes and subclasses herein.

In some embodiments, the present invention provides a compound of either of the formulae V-a-ix or V-a-x:

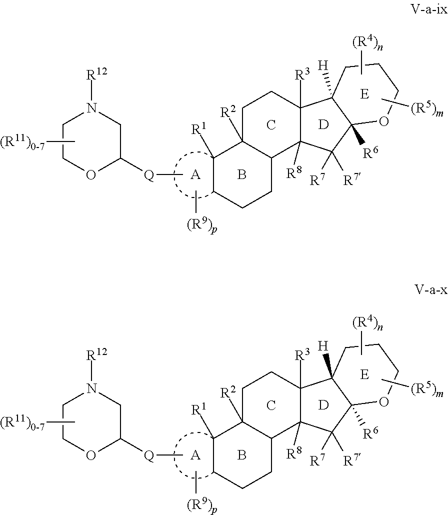

or a pharmaceutically acceptable salt thereof, wherein each variable is defined above and in classes and subclasses herein.

In some embodiments, the present invention provides a compound of the formula V-a-xi:

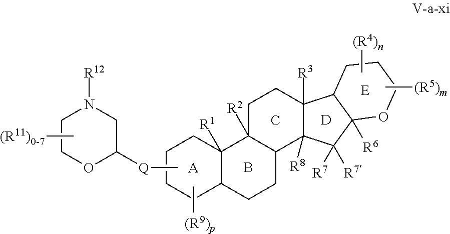

or a pharmaceutically acceptable salt thereof, wherein each variable is defined above and in classes and subclasses herein.

In some embodiments, the present invention provides a compound of either of the formulae V-a-xii or V-a-xiii:

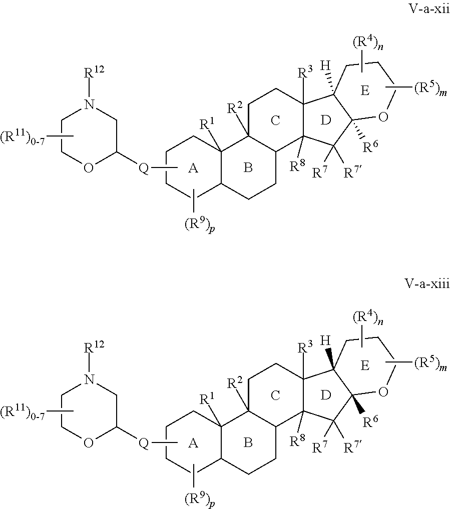

or a pharmaceutically acceptable salt thereof, wherein each variable is defined above and in classes and subclasses herein.

In some embodiments, the present invention provides a compound of either of the formulae V-a-xiv or V-a-xv:

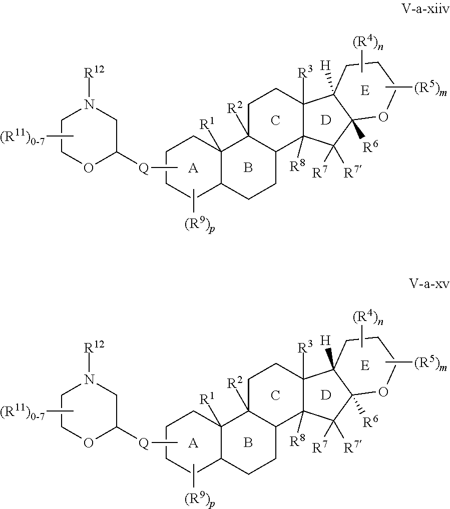

or a pharmaceutically acceptable salt thereof, wherein each variable is defined above and in classes and subclasses herein.

In some embodiments, the present invention provides a compound of either of the formulae V-a-xvi(a) or V-a-xvi(b):

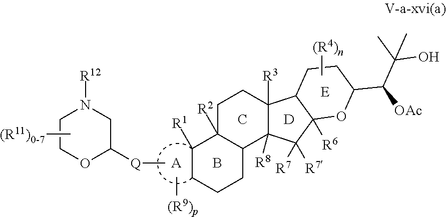

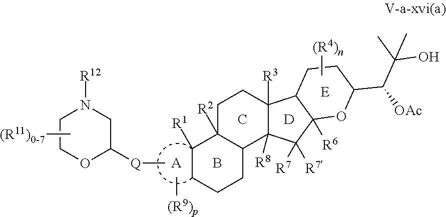

or a pharmaceutically acceptable salt thereof, wherein each variable is defined above and in classes and subclasses herein.

In some embodiments, the present invention provides a compound of either of the formulae V-a-xvii or V-a-xviii:

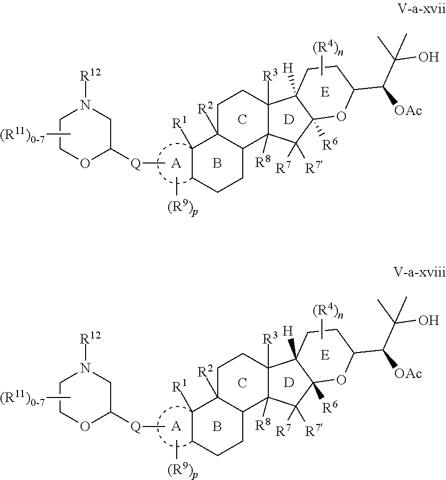

or a pharmaceutically acceptable salt thereof, wherein each variable is defined above and in classes and subclasses herein.

In some embodiments, the present invention provides a compound of either of the formulae V-a-xix or V-a-xx:

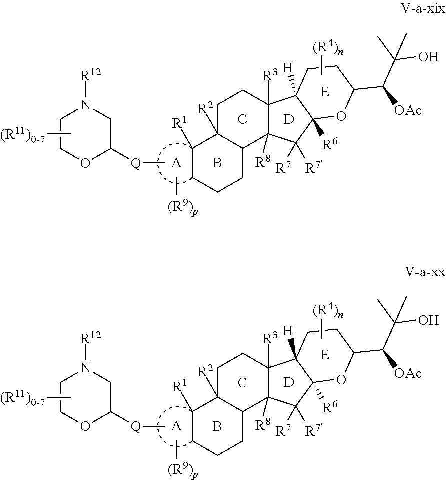

or a pharmaceutically acceptable salt thereof, wherein each variable is defined above and in classes and subclasses herein.

In some embodiments, the present invention provides a compound of the formula V-a-xxi:

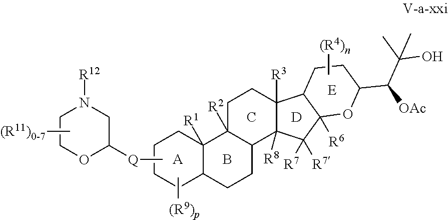

or a pharmaceutically acceptable salt thereof, wherein each variable is defined above and in classes and subclasses herein.

In some embodiments, the present invention provides a compound of either of the formulae V-a-xxii or V-a-xxiii:

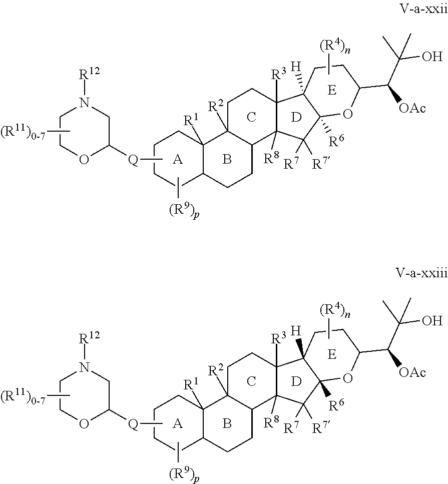

or a pharmaceutically acceptable salt thereof, wherein each variable is defined above and in classes and subclasses herein.

In some embodiments, the present invention provides a compound of either of the formulae V-a-xxiv or V-a-xxv:

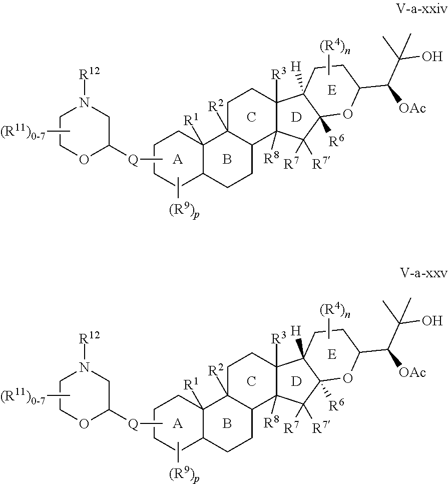

or a pharmaceutically acceptable salt thereof, wherein each variable is defined above and in classes and subclasses herein.

In some embodiments, the present invention provides a compound of formula V-a-xxvi:

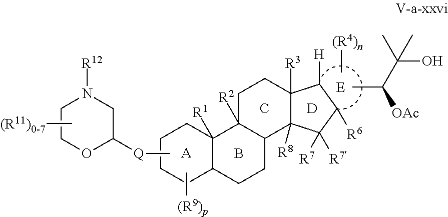

or a pharmaceutically acceptable salt thereof, wherein each variable is defined above and in classes and subclasses herein.

In some embodiments, the present invention provides a compound of either of the formulae V-a-xxvii or V-a-xxviii:

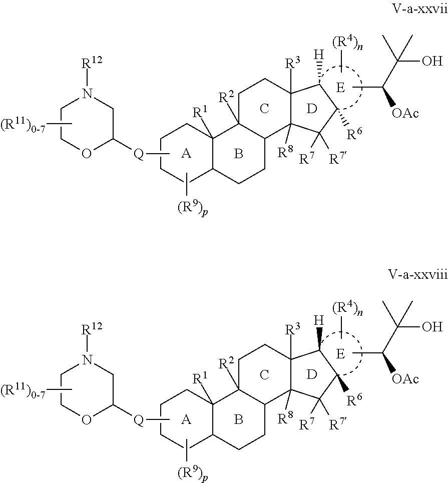

or a pharmaceutically acceptable salt thereof, wherein each variable is defined above and in classes and subclasses herein.

In some embodiments, the present invention provides a compound of either of the formulae V-a-xxix or V-a-xxx:

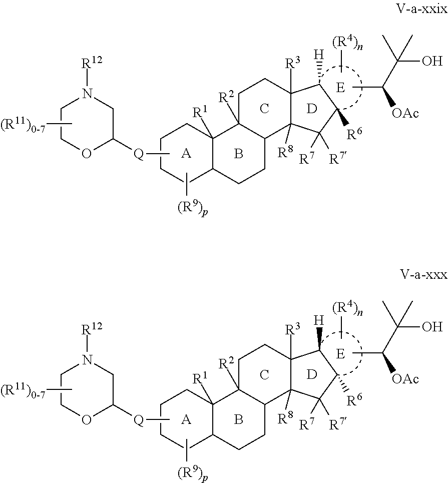

or a pharmaceutically acceptable salt thereof, wherein each variable is defined above and in classes and subclasses herein.

In some embodiments, the present invention provides a compound of the formula V-a-xxxi:

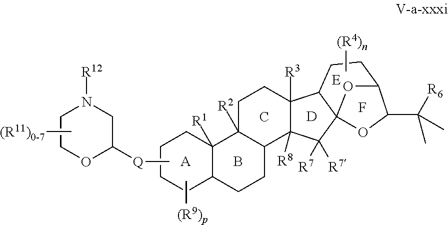

or a pharmaceutically acceptable salt thereof, wherein each variable is defined above and in classes and subclasses herein.

In certain embodiments, the R¹⁰ group of formula I is a sugar-containing group. Such sugar-containing groups are well known to one of ordinary skill in the art and include those described in detail in “Essentials of Glycobiology” Edited by Varki, A., et al., Cold Spring Harbor Laboratory Press, Cold Spring Harbor, N.Y. 2002.

In some embodiments, the R¹⁰ group of formula I is a glycoside.

In some embodiments, the present invention provides a compound of the formula V-b:

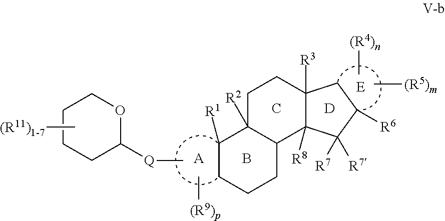

or a pharmaceutically acceptable salt thereof, wherein each variable is defined above and in classes and subclasses herein.

In some embodiments, R¹⁰ is of one of the following formulae:

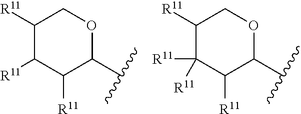

wherein each R¹¹ is as defined above and described herein. In certain embodiments, R¹⁰ is of one of the formulae shown above wherein one or more R¹¹ is independently fluorine. In certain embodiments, R¹⁰ is of one of the formulae shown above wherein one or more R¹¹ is independently —N(R)₂ or —CH₂N(R)₂. In certain embodiments, R¹⁰ is of one of the formulae shown above wherein one or more R¹¹ is independently OR, wherein R is optionally substituted C₁₋₆ aliphatic. Exemplary such optionally substituted C₁₋₆ aliphatic groups include optionally substituted alkyl or cycloalkyl groups selected from methyl, ethyl, CF₃, CF₂CF₃, cyclopropyl, cyclopentyl, and cyclohexyl.

In some embodiments, R¹⁰ is of the following formula:

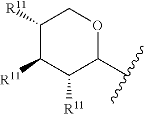

wherein each R¹¹ is as defined above and described herein.

In some embodiments, R¹⁰ is of one of the following formulae:

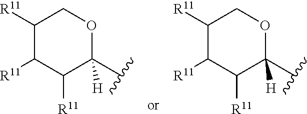

wherein each R¹¹ is as defined above and described herein.

In some embodiments, R¹⁰ is of one of the following formulae:

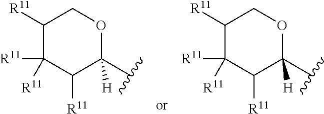

wherein each R¹¹ is as defined above and described herein.

In some embodiments, R¹⁰ is of one of the following formulae:

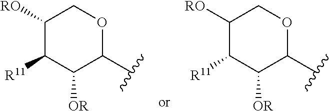

wherein each R and R¹¹ are as defined above and described herein.

In some embodiments, R¹⁰ is of the following formula:

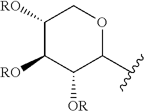

wherein each R is as defined above and described herein.

In some embodiments, R¹⁰ is of any of the following formulae:

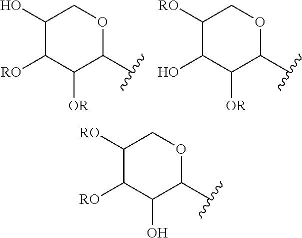

wherein each R is as defined above and described herein.

In some embodiments, R¹⁰ is of any of the following formulae:

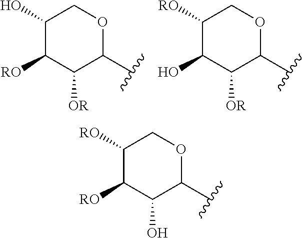

wherein each R is as defined above and described herein.

In some embodiments, R¹⁰ is of any of the following formulae:

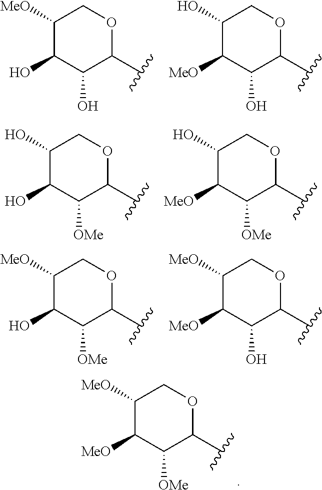

In some embodiments, R¹⁰ is of the following formula:

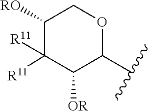

wherein each R and R¹¹ are as defined above and described herein. In certain embodiments, R¹⁰ is of the formula shown above wherein one or more R¹¹ is independently OR. In certain embodiments, R¹⁰ is of the formula shown above wherein one or more R¹¹ is independently OH. In certain embodiments, R¹⁰ is of the formula shown above wherein one or more R¹¹ is independently an optionally substituted C₁₋₆ aliphatic group. In certain embodiments, R¹⁰ is of the formula shown above wherein one or more R¹¹ is independently an optionally substituted aliphatic moiety of the formula —(CH₂)₁₋₆N(R)₂. In certain embodiments, R¹⁰ is of the formula shown above wherein one R¹¹ is independently an optionally substituted aliphatic moiety of the formula —CH₂N(R)₂.

Exemplary R¹⁰ groups include arabinopyranosides and xylopyranosides. In certain embodiments, R¹⁰ is a xylopyranoside. In certain embodiments, R¹⁰ is an arabinopyranoside. In still other embodiments, R¹⁰ is

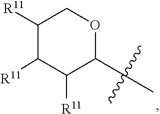

wherein each R¹¹ is as defined above and described herein. According to another embodiment, R¹⁰ is

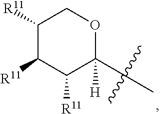

wherein each R¹¹ is as defined above and described herein. Yet another embodiment provides a compound of formula I wherein R¹⁰ is

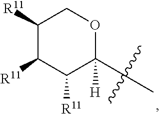

wherein each R¹¹ is as defined above and described herein. In some embodiments, R¹⁰ is

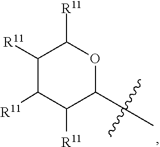

wherein each R¹¹ is as defined above and described herein. In certain embodiments, R¹⁰ is

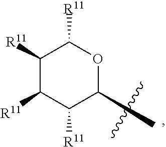

wherein each R¹¹ is as defined above and described herein. In certain embodiments, R¹⁰ is of any of the formulae shown above, wherein one or more R¹¹ groups is fluorine. In certain embodiments, R¹⁰ is of any of the formulae shown above, wherein two R¹¹ groups are fluorine. In certain embodiments, R¹⁰ is of any of the formulae shown above, wherein one or more R¹¹ groups is OH. In certain embodiments, R¹⁰ is of any of the formulae shown above, wherein two or more R¹¹ groups is OH. In certain embodiments, R¹⁰ is of any of the formulae shown above, wherein each R¹¹ group is OH. In certain embodiments, R¹⁰ is of any of the formulae shown above, wherein one or more R¹¹ groups is OCF₃. In certain embodiments, R¹⁰ is of any of the formulae shown above, wherein one or more R¹¹ groups is OMe. In certain embodiments, R¹⁰ is of any of the formulae shown above, wherein each R¹¹ group is OMe.

According to another aspect of the present invention, the R¹⁰ group of formula I is a sugar-mimetic. Such sugar-mimetics are well known to one of ordinary skill in the art and include those described in detail in “Essentials of Glycobiology.” For example, sugar-mimetic groups contemplated by the present invention include cyclitols and the like. In certain embodiments, R¹⁰ is a cyclitol moiety, wherein said cyclitol is a cycloalkane containing one hydroxyl group on each of three or more ring atoms, as defined by IUPAC convention. In other embodiments, such cyclitol moieties include inositols such as scyllo-inositol.

Suitable sugar-like moieties of the R¹⁰ group of formula I include acyclic sugar groups. Such groups include linear alkylols and erythritols, to name but a few. It will be appreciated that sugar groups can exist in either cyclic or acyclic form. Accordingly, acyclic forms of a sugar group are contemplated by the present invention as a suitable sugar-like moiety of the R¹⁰ group of formula I.

### 7. Additional R¹⁰ Embodiments

In certain embodiments, the R¹⁰ group of formula I is a detectable moiety. In other embodiments, the R¹⁰ group of formula I is a fluorescent label, fluorescent dye, or fluorophore as defined herein, supra. In certain embodiments, the detectable moiety comprises biotin.

For example, in some embodiments, the R¹⁰ group of formula I is as depicted below

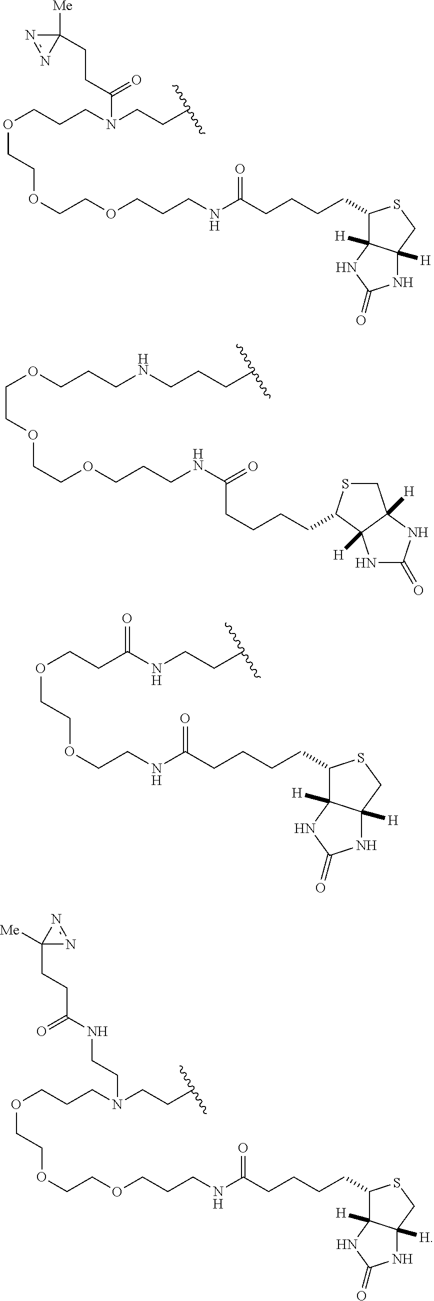

According to another aspect of the present invention, the R¹⁰ group of formula I is a polymer residue. Polymer residues are well known in the art and include those described in detail in “Chemistry of Protein Conjugation and Cross-Linking” Shan S. Wong, CRC Press. Boca Raton, Fla. 1991. Suitable polymer residues of the R¹⁰ group of formula I include poly(alkylene oxides), such as PEG, poly(amino acids), and other polymer residues capable of conjugation to a compound of the present invention.

As defined generally above, the R¹⁰ group of formula I is, inter alia, a suitably protected hydroxyl group, a suitably protected thiol group, or a suitably protected amino group. Hydroxyl protecting groups are well known in the art and include those described in detail in Protecting Groups in Organic Synthesis, T. W. Greene and P. G. M. Wuts, 3^{rd} edition, John Wiley & Sons, 1999, the entirety of which is incorporated herein by reference. Examples of suitable hydroxyl protecting groups of the R¹⁰ group of formula I further include, but are not limited to, esters, allyl ethers, ethers, silyl ethers, alkyl ethers, arylalkyl ethers, and alkoxyalkyl ethers. Examples of such esters include formates, acetates, carbonates, and sulfonates. Specific examples include formate, benzoyl formate, chloroacetate, trifluoroacetate, methoxyacetate, triphenylmethoxyacetate, p-chlorophenoxyacetate, 3-phenylpropionate, 4-oxopentanoate, 4,4-(ethylenedithio)pentanoate, pivaloate (trimethylacetyl), crotonate, 4-methoxy-crotonate, benzoate, p-benzylbenzoate, 2,4,6-trimethylbenzoate, carbonates such as methyl, 9-fluorenylmethyl, ethyl, 2,2,2-trichloroethyl, 2-(trimethylsilyl)ethyl, 2-(phenylsulfonyl)ethyl, vinyl, allyl, and p-nitrobenzyl. Examples of such silyl ethers include trimethylsilyl, triethylsilyl, t-butyldimethylsilyl, t-butyldiphenylsilyl, triisopropylsilyl, and other trialkylsilyl ethers. Alkyl ethers include methyl, benzyl, p-methoxybenzyl, 3,4-dimethoxybenzyl, trityl, t-butyl, allyl, and allyloxycarbonyl ethers or derivatives. Alkoxyalkyl ethers include acetals such as methoxymethyl, methylthiomethyl, (2-methoxyethoxy)methyl, benzyloxymethyl, beta-(trimethylsilyl)ethoxymethyl, and tetrahydropyranyl ethers. Examples of arylalkyl ethers include benzyl, p-methoxybenzyl (MPM), 3,4-dimethoxybenzyl, O-nitrobenzyl, p-nitrobenzyl, p-halobenzyl, 2,6-dichlorobenzyl, p-cyanobenzyl, and 2- and 4-picolyl.

Thiol protecting groups are well known in the art and include those described in detail in Protecting Groups in Organic Synthesis, T. W. Greene and P. G. M. Wuts, 3^{rd} edition, John Wiley & Sons, 1999, the entirety of which is incorporated herein by reference. Suitable thiol protecting groups of the R¹⁰ moiety of formula I include, but are not limited to, disulfides, thioethers, silyl thioethers, thioesters, thiocarbonates, thiocarbamates, and the like. Examples of such groups include, but are not limited to, alkyl thioethers, benzyl and substituted benzyl thioethers, triphenylmethyl thioethers, trichloroethoxycarbonyl, to name but a few.

According to another aspect of the present invention, the R¹⁰ moiety of formula I is a thiol protecting group that is removable under neutral conditions e.g. with AgNO₃, HgCl₂, and the like. Other neutral conditions include reduction using a suitable reducing agent. Suitable reducing agents include dithiothreitol (DTT), mercaptoethanol, dithionite, reduced glutathione, reduced glutaredoxin, reduced thioredoxin, substituted phosphines such as tris carboxyethyl phosphine (TCEP), and any other peptide or organic based reducing agent, or other reagents known to those of ordinary skill in the art. According to yet another aspect of the present invention, the R¹⁰ moiety of formula I is a thiol protecting group that is “photocleavable”. Such suitable thiol protecting groups are known in the art and include, but are not limited to, a nitrobenzyl group, a tetrahydropyranyl (THP) group, a trityl group, —CH₂SCH₃ (MTM), dimethylmethoxymethyl, or —CH₂—S—S-pyridin-2-yl. One of ordinary skill in the art would recognize that many of the suitable hydroxyl protecting groups, as described herein, are also suitable as thiol protecting groups.

In certain embodiments, the R¹⁰ group of formula I is a suitably protected amino group. Amino protecting groups are well known in the art and include those described in detail in Protecting Groups in Organic Synthesis, T. W. Greene and P. G. M. Wuts, 3^{rd} edition, John Wiley & Sons, 1999, the entirety of which is incorporated herein by reference. Suitable amino protecting groups of said R¹⁰ moiety further include, but are not limited to, aralkylamines, carbamates, cyclic imides, allyl amines, amides, and the like. Examples of such groups include t-butyloxycarbonyl (BOC), ethyloxycarbonyl, methyloxycarbonyl, trichloroethyloxycarbonyl, allyloxycarbonyl (Alloc), benzyloxocarbonyl (CBZ), allyl, phthalimide, benzyl (Bn), fluorenylmethylcarbonyl (Fmoc), formyl, acetyl, chloroacetyl, dichloroacetyl, trichloroacetyl, phenylacetyl, trifluoroacetyl, benzoyl, and the like. In certain embodiments, the amino protecting group of the R¹⁰ moiety is phthalimido. In still other embodiments, the amino protecting group of the R¹⁰ moiety is a tert-butyloxycarbonyl (BOC) group. In certain embodiments, the amino protecting group is a sulphone (SO₂R).

In some embodiments, R¹⁰ is SO₂R. In some embodiments, R¹⁰ is C(O)N(R)₂. In some embodiments, R¹⁰ is CO₂R.

In some embodiments, Q is a valence bond and R¹⁰ is fluorine. In other embodiments, Q is a valence bond and R¹⁰ hydrogen. In other embodiments, Q is a valence bond and R¹⁰ is R, OR or N(R)₂.

In some embodiments, Q-R¹⁰ of formula I is of any of the following formulae:

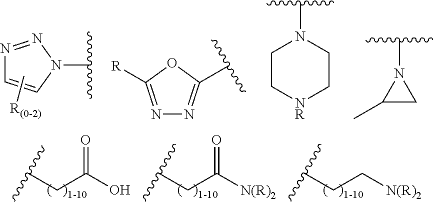

wherein R is as defined above and described herein.

### 8. Ring A Embodiments

As defined generally above, Ring A is a 4-7 membered saturated or partially unsaturated ring having 0-2 heteroatoms independently selected from nitrogen, oxygen, or sulfur.

In some embodiments, Ring A is a 4-7 membered saturated ring having 0-2 heteroatoms independently selected from nitrogen, oxygen, or sulfur. In some embodiments, Ring A is a 4 membered saturated ring having 1-2 heteroatoms independently selected from nitrogen, oxygen, or sulfur. In some embodiments, Ring A is a 4 membered saturated carbocycle. In some embodiments, Ring A is a 5 membered saturated ring having 1-2 heteroatoms independently selected from nitrogen, oxygen, or sulfur. In some embodiments, Ring A is a 5 membered saturated carbocycle. In some embodiments, Ring A is a 6 membered saturated ring having 1-2 heteroatoms independently selected from nitrogen, oxygen, or sulfur. In some embodiments, Ring A is a 6 membered saturated carbocycle. In some embodiments, Ring A is a 7 membered saturated ring having 1-2 heteroatoms independently selected from nitrogen, oxygen, or sulfur. In some embodiments, Ring A is a 7 membered saturated carbocycle.

In some embodiments, Ring A is a 5-7 membered partially unsaturated ring having 0-2 heteroatoms independently selected from nitrogen, oxygen, or sulfur. In some embodiments, Ring A is a 5 membered partially unsaturated ring having 1-2 heteroatoms independently selected from nitrogen, oxygen, or sulfur. In some embodiments, Ring A is a 5 membered partially unsaturated carbocycle. In some embodiments, Ring A is a 6 membered partially unsaturated ring having 1-2 heteroatoms independently selected from nitrogen, oxygen, or sulfur. In some embodiments, Ring A is a 6 membered partially unsaturated carbocycle. In some embodiments, Ring A is a 7 membered partially unsaturated ring having 1-2 heteroatoms independently selected from nitrogen, oxygen, or sulfur. In some embodiments, Ring A is a 7 membered partially unsaturated carbocycle.

As defined generally above and herein, p is 0-4. In some embodiments, p is 0. In some embodiments, p is 1. In some embodiments, p is 2. In some embodiments, p is 3. In some embodiments, p is 4.

As defined generally above, each R⁹ is independently selected from halogen, R, OR, SR, or N(R)₂, or:

wherein two R⁹ are optionally taken together to form a 3-7 membered saturated or partially unsaturated ring having 0-4 heteroatoms independently selected from nitrogen, oxygen, or sulfur, or:

wherein two R⁹ on the same carbon atom are optionally taken together to form an oxo moiety, an oxime, an optionally substituted hydrazone, an optionally substituted imine, or an optionally substituted C₂₋₆ alkylidene.

In some embodiments, each R⁹ is independently selected from halogen, R, OR, SR, or N(R)₂.

In certain embodiments, two R⁹ are taken together to form a 3-7 membered saturated ring having 1-2 heteroatoms independently selected from nitrogen, oxygen, or sulfur. In certain embodiments, two R⁹ are taken together to form a 3-7 membered saturated carbocycle. In certain embodiments, two R⁹ on the same carbon are taken together to form a 3-7 membered saturated or partially unsaturated spirocycle having 0-2 heteroatoms independently selected from nitrogen, oxygen, or sulfur. In certain embodiments, two R⁹ are taken together to form a 5-6 membered partially unsaturated ring having 1-2 heteroatoms independently selected from nitrogen, oxygen, or sulfur. In certain embodiments, two R⁹ are taken together to form a 5-6 membered partially unsaturated carbocycle. In some embodiments, two R⁹ on the same carbon atom are optionally taken together to form an oxo moiety.

In some embodiments, the present invention provides a compound of the formula V-c:

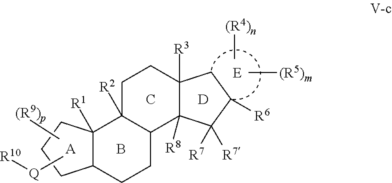

or a pharmaceutically acceptable salt thereof, wherein each variable is defined above and in classes and subclasses herein.

In some embodiments, Ring A is a 5 membered saturated monocyclic ring having the following formula:

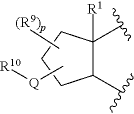

wherein each of R¹, R⁹, R¹⁰, p, and Q are as defined above and described herein.

In some embodiments, Ring A is of the following formula:

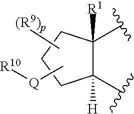

wherein each of R¹, R⁹, R¹⁰, p, and Q are as defined above and described herein.

In some embodiments, Ring A is of the following formula:

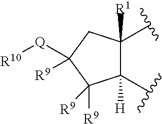

wherein each of R¹, R⁹, R¹⁰, and Q are as defined above and described herein.

In some embodiments, Ring A is of the following formula:

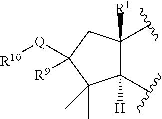

wherein each of R¹, R⁹, R¹⁰, and Q are as defined above and described herein.

In some embodiments, Ring A is of either of the following formulae:

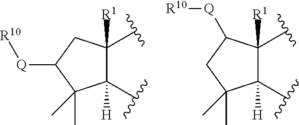

wherein each of R¹, R¹⁰, and Q are as defined above and described herein.

In some embodiments, Ring A is of any of the following formulae:

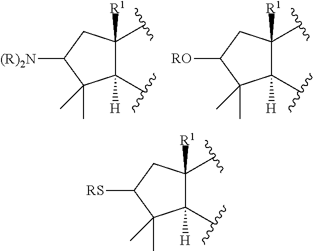

wherein each of R¹ and R are as defined above and described herein.

In some embodiments, a the present invention provides a compound of the formula V-d:

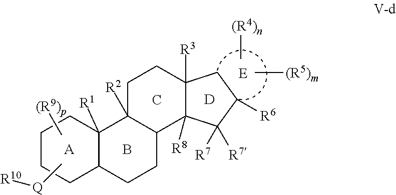

or a pharmaceutically acceptable salt thereof, wherein each variable is defined above and in classes and subclasses herein.

In some embodiments, the compound is of the following formula:

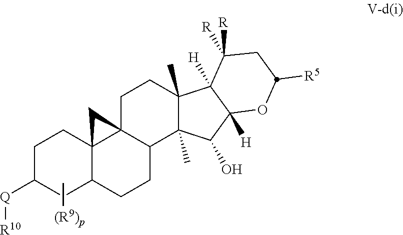

wherein each variable is defined above and in classes and subclasses herein.

In some embodiments, the compound is of the following formula:

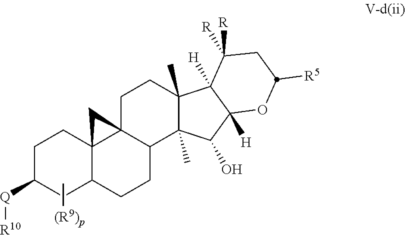

wherein each variable is defined above and in classes and subclasses herein.

In some embodiments, Ring A is a 6 membered saturated monocyclic ring having the following formula:

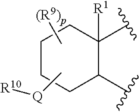

wherein each of R¹, R⁹, R¹⁰, p, and Q are as defined above and described herein.

In some embodiments, Ring A is of the following formula:

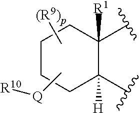

wherein each of R¹, R⁹, R¹⁰, p, and Q are as defined above and described herein.

In some embodiments, Ring A is of the following formula:

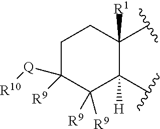

wherein each of R¹, R⁹, R¹⁰, and Q are as defined above and described herein.

In some embodiments, Ring A is of any one of the following formulae:

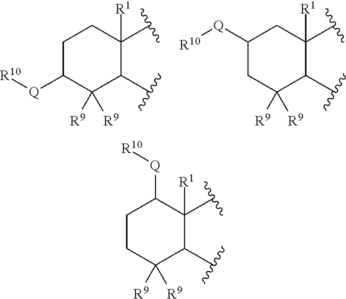

wherein each of R¹, R⁹, R¹⁰, and Q are as defined above and described herein.

In some embodiments, Ring A is of any one of the following formulae:

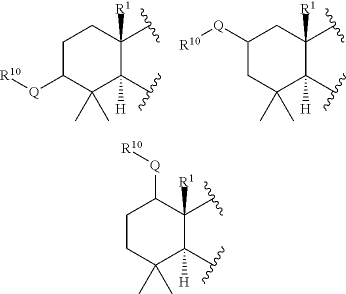

wherein each of R¹, R¹⁰, and Q are as defined above and described herein.

In some embodiments, Ring A is a 7 membered saturated ring containing one or more nitrogens. In certain embodiments, Ring A is an azepane. In certain embodiments, Ring A is an azepane substituted with 2-4 R⁹ groups. In certain embodiments, Ring A is an azepanone. In certain embodiments, Ring A is an azepanone substituted with 2-4 R⁹ groups.

In some embodiments, a the present invention provides a compound of the formula V-e:

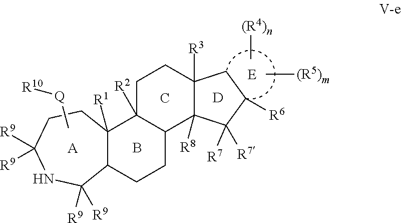

or a pharmaceutically acceptable salt thereof, wherein each variable is defined above and in classes and subclasses herein.

In certain embodiments Ring A is of the following formula:

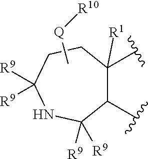

wherein each of R¹, R⁹, R¹⁰, and Q are as defined above and described herein.

In certain embodiments Ring A is of either the following formulae:

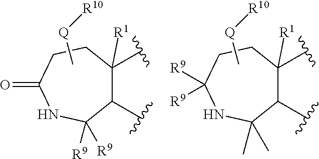

wherein each of R¹, R⁹, R¹⁰, and Q are as defined above and described herein.

In certain embodiments Ring A is of either the following formulae:

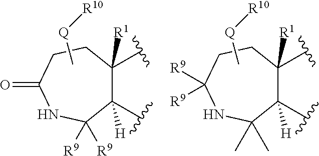

wherein each of R¹, R⁹, R¹⁰, and Q are as defined above and described herein.

### 9. Ring D Embodiments

As defined generally above, R³ and R⁸ are each independently selected from halogen, CN, R, OR, a suitably protected hydroxyl group, SR, a suitably protected thiol group, S(O)R, SO₂R, OSO₂R, N(R)₂, a suitably protected amino group, N(R)C(O)R, N(R)C(O)C(O)R, N(R)C(O)N(R)₂, N(R)C(O)OR, C(O)OR, OC(O)R, C(O)N(R)₂, or OC(O)N(R)₂.

In certain embodiments, R³ or R⁸ are each independently selected from halogen, R, OR, or a suitably protected hydroxyl group. In certain embodiments, at least one of R³ or R⁸ is independently selected from SR, a suitably protected thiol group, S(O)R, SO₂R, or OSO₂R. In certain embodiments, at least one of R³ or R⁸ is independently selected from N(R)₂, a suitably protected amino group, N(R)C(O)R, N(R)C(O)C(O)R, N(R)C(O)N(R)₂, or N(R)C(O)OR. In certain embodiments, at least one of R³ or R⁸ is independently selected from C(O)OR, OC(O)R, C(O)N(R)₂, or OC(O)N(R)₂. In certain embodiments, at least one of R³ or R⁸ is independently R. In certain embodiments, at least one of R³ or R⁸ is independently hydrogen, fluorine, methyl, or trifluoromethyl.

As defined generally above, each of R⁷ and R^{7′} is independently selected from halogen, CN, N₃, R, OR, a suitably protected hydroxyl group, SR, a suitably protected thiol group, SO₂R, OSO₂R, N(R)₂, a suitably protected amino group, N(R)C(O)R, N(R)C(O)C(O)R, N(R)C(O)N(R)₂, N(R)C(O)OR, C(O)OR, OC(O)R, C(O)N(R)₂, or OC(O)N(R)₂, or:
R⁷ and R^{7′} are taken together to form an oxo moiety, an oxime, an optionally substituted hydrazone, an optionally substituted imine, an optionally substituted C₂₋₆ alkylidene, or an optionally substituted 3-8 membered saturated or partially unsaturated spirocycle having 0-4 heteroatoms independently selected from nitrogen, oxygen, or sulfur, or:
R⁶ and R⁷ or R⁶ and R^{7′} are optionally taken together to form an optionally substituted 3-8 membered saturated or partially unsaturated ring having 0-4 heteroatoms selected from nitrogen, oxygen, or sulfur.

In some embodiments, R⁷ and R^{7′} are taken together to form an oxo moiety. In some embodiments, R⁷ and R^{7′} are taken together to form an optionally substituted 3-8 membered saturated or partially unsaturated spirocycle having 0-4 heteroatoms independently selected from nitrogen, oxygen, or sulfur.

In some embodiments, R⁷ and R^{7′} are taken together to form an optionally substituted 3-8 membered saturated spirocycle having 0-4 heteroatoms independently selected from nitrogen, oxygen, or sulfur. In some embodiments, R⁷ and R^{7′} are taken together to form an optionally substituted 3-8 membered saturated spirocycle having 1-2 heteroatoms independently selected from nitrogen, oxygen, or sulfur. In some embodiments, R⁷ and R^{7′} are taken together to form an optionally substituted 5-6 membered saturated spirocycle having 0-4 heteroatoms independently selected from nitrogen, oxygen, or sulfur. In some embodiments, R⁷ and R^{7′} are taken together to form an optionally substituted 5-6 membered saturated spirocycle having 1-2 heteroatoms independently selected from nitrogen, oxygen, or sulfur.

In some embodiments, R⁷ and R^{7′} are taken together to form an optionally substituted 5-8 membered partially unsaturated spirocycle having 0-4 heteroatoms independently selected from nitrogen, oxygen, or sulfur. In some embodiments, R⁷ and R^{7′} are taken together to form an optionally substituted 5-8 membered partially unsaturated spirocycle having 1-2 heteroatoms independently selected from nitrogen, oxygen, or sulfur.

In some embodiments, R⁶ and R⁷ are optionally taken together to form an optionally substituted 3-8 membered saturated or partially unsaturated monocyclic ring having 0-4 heteroatoms selected from nitrogen, oxygen, or sulfur.

In some embodiments, R⁶ and R⁷ are optionally taken together to form an optionally substituted 3-8 membered saturated monocyclic ring having 1-3 heteroatoms independently selected from nitrogen, oxygen, or sulfur. In certain embodiments, R⁶ and R⁷ are optionally taken together to form an optionally substituted 3-8 membered saturated monocyclic carbocycle. In certain embodiments, R⁶ and R⁷ are optionally taken together to form an optionally substituted 5-6 membered saturated monocyclic ring having 1-2 heteroatoms independently selected from nitrogen, oxygen, or sulfur. In certain embodiments, R⁶ and R⁷ are optionally taken together to form an optionally substituted 5-6 membered saturated monocyclic carbocycle. In certain embodiments, R⁶ and R⁷ are optionally taken together to form an optionally substituted 7 membered saturated monocyclic ring having 1-2 heteroatoms independently selected from nitrogen, oxygen, or sulfur. In certain embodiments, R⁶ and R⁷ are optionally taken together to form an optionally substituted 7 membered saturated monocyclic carbocycle.

In certain embodiments, R⁶ and R⁷ are optionally taken together to form an optionally substituted 3-8 membered partially unsaturated monocyclic ring having 1-3 heteroatoms independently selected from nitrogen, oxygen, or sulfur. In certain embodiments, R⁶ and R⁷ are optionally taken together to form an optionally substituted 3-8 membered partially unsaturated monocyclic carbocycle. In certain embodiments, R⁶ and R⁷ are optionally taken together to form an optionally substituted 5-6 membered partially unsaturated monocyclic ring having 1-2 heteroatoms independently selected from nitrogen, oxygen, or sulfur. In certain embodiments, R⁶ and R⁷ are optionally taken together to form an optionally substituted 5-6 membered partially unsaturated monocyclic carbocycle.

In some embodiments, R⁶ and R^{7′} are optionally taken together to form an optionally substituted 3-8 membered saturated or partially unsaturated having 0-4 heteroatoms selected from nitrogen, oxygen, or sulfur.

In some embodiments, R⁶ and R^{7′} are optionally taken together to form an optionally substituted 3-8 membered saturated monocyclic ring having 1-3 heteroatoms independently selected from nitrogen, oxygen, or sulfur. In certain embodiments, R⁶ and R^{7′} are optionally taken together to form an optionally substituted 3-8 membered saturated monocyclic carbocycle. In certain embodiments, R⁶ and R^{7′} are optionally taken together to form an optionally substituted 5-6 membered saturated monocyclic ring having 1-2 heteroatoms independently selected from nitrogen, oxygen, or sulfur. In certain embodiments, R⁶ and R^{7′} are optionally taken together to form an optionally substituted 5-6 membered saturated monocyclic carbocycle. In certain embodiments, R⁶ and R^{7′} are optionally taken together to form an optionally substituted 7 membered saturated monocyclic ring having 1-2 heteroatoms independently selected from nitrogen, oxygen, or sulfur. In certain embodiments, R⁶ and R^{7′} are optionally taken together to form an optionally substituted 7 membered saturated monocyclic carbocycle.

In certain embodiments, R⁶ and R^{7″} are optionally taken together to form an optionally substituted 3-8 membered partially unsaturated monocyclic ring having 1-3 heteroatoms independently selected from nitrogen, oxygen, or sulfur. In certain embodiments, R⁶ and R^{7″} are optionally taken together to form an optionally substituted 3-8 membered partially unsaturated monocyclic carbocycle. In certain embodiments, R⁶ and R^{7″} are optionally taken together to form an optionally substituted 5-6 membered partially unsaturated monocyclic ring having 1-2 heteroatoms independently selected from nitrogen, oxygen, or sulfur. In certain embodiments, R⁶ and R^{7″} are optionally taken together to form an optionally substituted 5-6 membered partially unsaturated monocyclic carbocycle.

In other embodiments, one of R⁷ and R^{7′} is OR and the other of R⁷ and R^{7′} is CN, N₃, C₁₋₆ alkyl, C₁₋₆ alkenyl, or C₁₋₆ alkynyl.

In certain embodiments, the R⁷ group of formula I is halogen. In some embodiments, R⁷ is fluoro. In certain embodiments, R⁷ is R. In some embodiments, R⁷ is R wherein R is hydrogen. In other embodiments, R⁷ is R wherein R is optionally substituted C₁₋₆ alkyl. In certain embodiments, the R⁷ group of formula I is OR. In some embodiments, R⁷ is OR wherein R is hydrogen. In other embodiments, R⁷ is OR wherein R is C₁₋₆ alkyl. In some embodiments, R⁷ is N(R)₂. In certain embodiments, R⁷ is NH₂.

In certain embodiments, the R^{7′} group of formula I is halogen. In some embodiments, R⁷ is fluoro. In certain embodiments, R^{7′} is R. In some embodiments, R^{7′} is R wherein R is hydrogen. In other embodiments, R^{7′} is R wherein R is optionally substituted C₁₋₆ alkyl. In certain embodiments, the R^{7′} group of formula I is OR. In some embodiments, R⁷ is OR wherein R is hydrogen. In certain embodiments, R^{7′} is OR wherein R is C₁₋₆ alkyl.

In some embodiments, a the present invention provides a compound of the formula V-f:

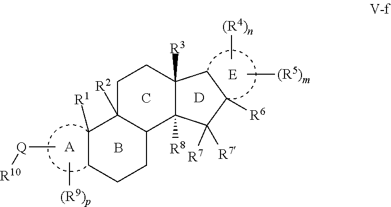

or a pharmaceutically acceptable salt thereof, wherein each variable is defined above and in classes and subclasses herein.

In some embodiments, Ring D is of either of the following formulae:

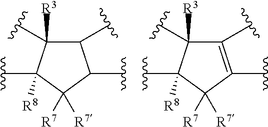

wherein each of R³, R⁷, R^{7′}, and R⁸ are as defined above and described herein.

In some embodiments, Ring D is of any of the following formulae:

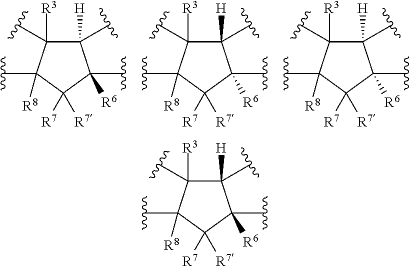

wherein each of R³, R⁶, R⁷, R^{7′}, and R⁸ are as defined above and described herein.

In some embodiments, Ring D is of any of the following formulae:

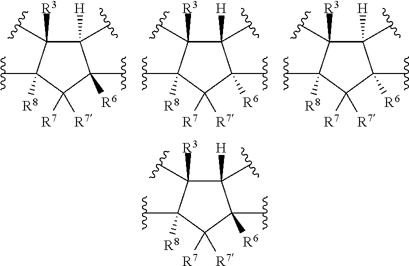

wherein each of R³, R⁶, R⁷, R^{7′}, and R⁸ are as defined above and described herein.

In some embodiments, Ring D is of any of the following formulae:

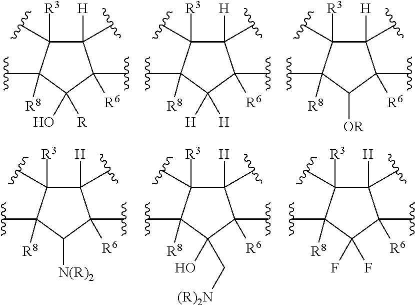

wherein each of R, R³, R⁶, and R⁸ are as defined above and described herein.

In some embodiments, Ring D is of either of the following formulae:

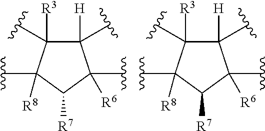

wherein each of R³, R⁶, R⁷, and R⁸ are as defined above and described herein.

In some embodiments, Ring D is of either of the following formulae:

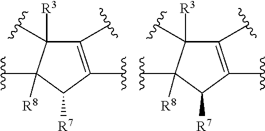

wherein each of R³, R⁷, and R⁸ are as defined above and described herein.

In some embodiments, Ring D is of any of the following formulae:

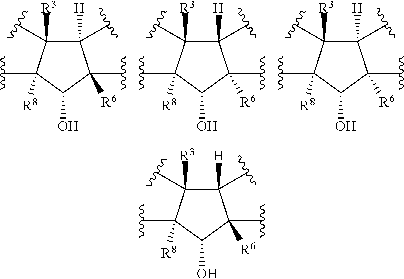

wherein each of R³, R⁶, and R⁸ are as defined above and described herein.

In some embodiments, Ring D is of any of the following formulae:

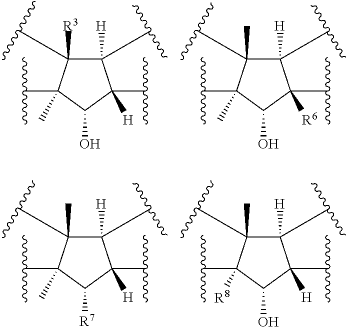

wherein each of R³, R⁶, R⁷, and R⁸ are as defined above and described herein.

### 10. Ring E Embodiments

As described generally above and herein, Ring E is a 4-7 membered saturated, partially unsaturated, or aromatic ring having 0-2 heteroatoms independently selected from nitrogen, oxygen, or sulfur. In certain embodiments wherein Ring E contains sulfur, the sulfur may optionally exist in an oxidized state, i.e., a sulfoxide, sulfone, or sulfate. Similarly, in certain embodiments wherein Ring E contains nitrogen, the nitrogen may optionally exist in an oxidized state such as, for instance, an n-oxide.

In some embodiments, Ring E is a 4-7 membered saturated ring having 0-2 heteroatoms independently selected from nitrogen, oxygen, or sulfur. In certain embodiments, Ring E is a 4 membered saturated ring having 1-2 heteroatoms independently selected from nitrogen, oxygen, or sulfur. In certain embodiments, Ring E is a 4 membered saturated carbocycle. In certain embodiments, Ring E is a 5 membered saturated ring having 1-2 heteroatoms independently selected from nitrogen, oxygen, or sulfur. In certain embodiments, Ring E is a 5 membered saturated carbocycle. In certain embodiments, Ring E is a 6 membered saturated ring having 1-2 heteroatoms independently selected from nitrogen, oxygen, or sulfur. In certain embodiments, Ring E is a 6 membered saturated carbocycle. In certain embodiments, Ring E is a 7 membered saturated ring having 1-2 heteroatoms independently selected from nitrogen, oxygen, or sulfur. In certain embodiments, Ring E is a 7 membered saturated carbocycle.

In certain embodiments, Ring E is an optionally substituted 5-7 membered saturated heterocyclic or carbocyclic ring selected from the group consisting of cyclopentane, dioxolane, oxazolidine, oxathiolane, imidazolidine, cyclohexane, morpholine, piperazine, piperidine, tetrahydropyran, dioxane, thiomorphaline, oxathiane, dithiane, oxepane, azepane, thiepane, oxapenone, azepanone, and thiepanone.

As defined generally above and herein, n is 0-4. In some embodiments, n is 0. In some embodiments, n is 1. In some embodiments, n is 2. In some embodiments, n is 3. In some embodiments, n is 4.

As defined generally above and herein, each R⁴ is independently selected from halogen, CN, R, OR, a suitably protected hydroxyl group, SR, a suitably protected thiol group, S(O)R, SO₂R, OSO₂R, N(R)₂, a suitably protected amino group, N(R)C(O)R, N(R)C(O)C(O)R, N(R)C(O)N(R)₂, N(R)C(O)OR, C(O)OR, OC(O)R, C(O)N(R)₂, or OC(O)N(R)₂, or:
two R⁴ on the same carbon are optionally taken together to form an optionally substituted 3-8 membered saturated or partially unsaturated spirofused ring having 0-4 heteroatoms independently selected from nitrogen, oxygen, or sulfur, or:
two R⁴ on the same carbon are optionally taken together to form an oxo moiety, an oxime, an optionally substituted hydrazone, an optionally substituted imine, or an optionally substituted C₂₋₆ alkylidene.

As defined generally above and herein, each R⁵ is independently T-C(R′)₃, T-C(R′)₂C(R″)₃, OR, a suitably protected hydroxyl group, SR, a suitably protected thiol group, S(O)R, SO₂R, OSO₂R, N(R)₂, a suitably protected amino group, N(R)C(O)R, N(R)C(O)C(O)R, N(R)C(O)N(R)₂, N(R)C(O)OR, C(O)OR, OC(O)R, C(O)N(R)₂, or OC(O)N(R)₂, an optionally substituted 3-8 membered saturated, partially unsaturated, or aryl monocyclic ring having 0-4 heteroatoms independently selected from nitrogen, oxygen, or sulfur, an optionally substituted 8-10 membered saturated, partially unsaturated, or aryl bicyclic ring having 0-4 heteroatoms independently selected from nitrogen, oxygen, or sulfur, or:
two R⁵ on the same carbon are optionally taken together to form an oxo moiety, an oxime, an optionally substituted hydrazone, an optionally substituted imine, an optionally substituted C₂₋₆ alkylidene, or an optionally substituted 3-8 membered saturated or partially unsaturated spirocycle having 0-4 heteroatoms independently selected from nitrogen, oxygen, or sulfur;
each T is independently a valence bond or an optionally substituted straight or branched, saturated or unsaturated, C₁₋₆ alkylene chain wherein up to two methylene units of T are optionally and independently replaced by —O—, —N(R)—, —S—, —C(O)—, —S(O)—, or —S(O)₂—;
each R′ and R″ is independently selected from halogen, R, OR, SR, S(O)R, SO₂R, OSO₂R, N(R)₂, N(R)C(O)R, N(R)C(O)C(O)R, N(R)C(O)N(R)₂, N(R)C(O)OR, N(R)S(O)R, N(R)SO₂R, N(R)SO₂OR C(O)OR, OC(O)R, C(O)N(R)₂, OC(O)N(R)₂, or an optionally substituted 3-8 membered saturated, partially unsaturated, or aryl monocyclic ring having 0-4 heteroatoms independently selected from nitrogen, oxygen, or sulfur, or an optionally substituted 8-10 membered saturated, partially unsaturated, or aryl bicyclic ring having 0-4 heteroatoms independently selected from nitrogen, oxygen, or sulfur, or:
two R′ are optionally taken together to form an oxo moiety, an oxime, an optionally substituted hydrazone, an optionally substituted imine, an optionally substituted C₂₋₆ alkylidene, or an optionally substituted 3-8 membered saturated or partially unsaturated ring having 0-4 heteroatoms independently selected from nitrogen, oxygen, or sulfur, or:
two R″ are optionally taken together to form an oxo moiety, an oxime, an optionally substituted hydrazone, an optionally substituted imine, an optionally substituted C₂₋₆ alkylidene, or an optionally substituted 3-8 membered saturated or partially unsaturated ring having 0-4 heteroatoms independently selected from nitrogen, oxygen, or sulfur.

As defined generally above and herein, m is 0-4. In some embodiments, m is 0. In some embodiments, m is 1. In some embodiments, m is 2. In some embodiments, m is 3. In some embodiments, m is 4.

In some embodiments, the present invention provides a compound of the formula V-g:

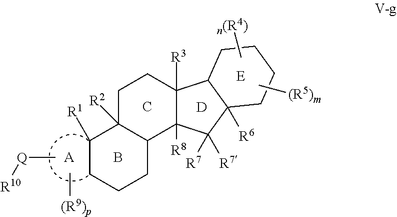

or a pharmaceutically acceptable salt thereof, wherein each variable is defined above and in classes and subclasses herein.

In some embodiments, Ring E is of any of the following formulae:

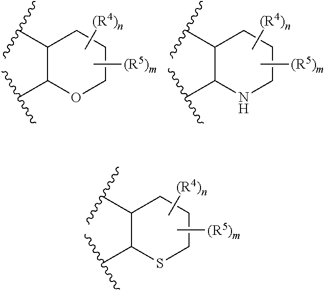

wherein each of R⁴, R⁵, n, and m are as defined above and described herein.

In some embodiments, Ring E is of any of the following formulae:

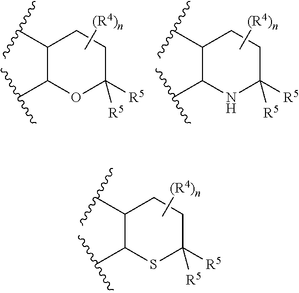

wherein each of R⁴, R⁵, and n are as defined above and described herein.

In some embodiments, Ring E is of any of the following formulae:

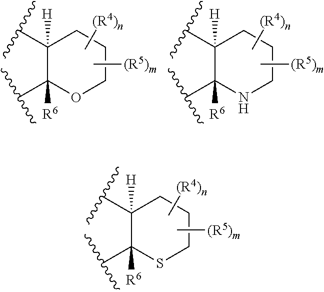

wherein each of R⁴, R⁵, R⁶, n, and m are as defined above and described herein.

In some embodiments, Ring E is of any of the following formulae:

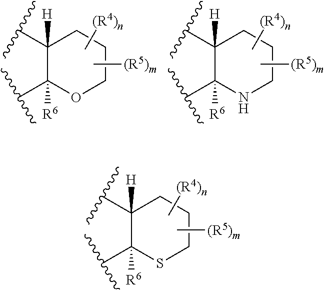

wherein each of R⁴, R⁵, R⁶, n, and m are as defined above and described herein.

In some embodiments, Ring E is of any of the following formulae:

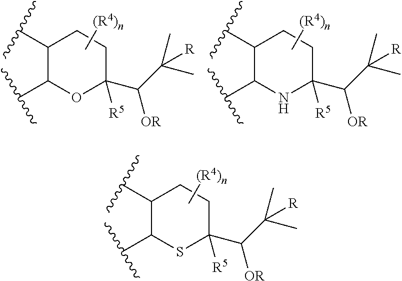

wherein each of R⁴, R⁵, R and n are as defined above and described herein.

In some embodiments, Ring E is of any of the following formulae:

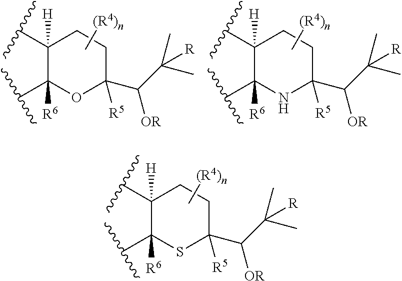

wherein each of R⁴, R⁵, R⁶, R and n are as defined above and described herein.

In some embodiments, Ring E is of any of the following formulae:

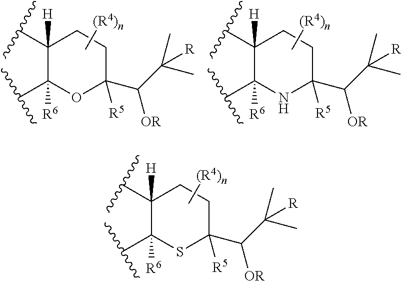

wherein each of R⁴, R⁵, R⁶, R and n are as defined above and described herein.

In some embodiments, Ring E is of any of the following formulae:

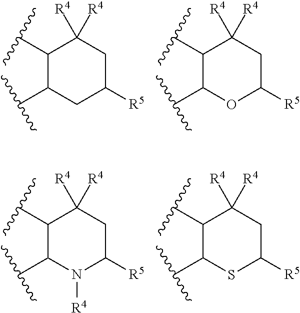

wherein each of R⁴ and R⁵ are as defined above and herein. In certain embodiments, Ring E is of one of the formulae shown above and one or more R⁴ is R. In certain embodiments, Ring E is of one of the formulae shown above and one or more R⁴ is methyl. In certain embodiments, Ring E is of one of the formulae shown above and one or more R⁴ is trifluoromethyl. In certain embodiments, Ring E is of one of the formulae shown above and one or more R⁴ is fluorine. In certain embodiments, Ring E is of one of the formulae shown above wherein two R⁴ on the same carbon form a gem-dimethyl group. In some embodiments, Ring E is of one of the formulae shown above and two R⁴ on the same carbon are taken together to form an oxo moiety, an oxime, an optionally substituted hydrazone, or an optionally substituted imine.

In some embodiments, Ring E is of any of the following formulae:

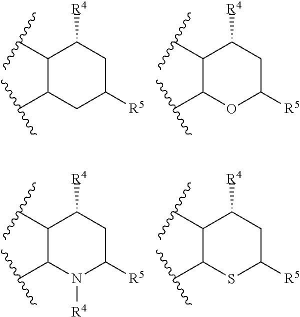

wherein each of R⁴ and R⁵ are as defined above and described herein.

In some embodiments, Ring E is of any of the following formulae:

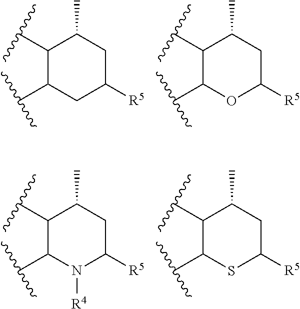

wherein each of R⁴ and R⁵ are as defined above and described herein.

In certain embodiments, Ring E is of the following formula:

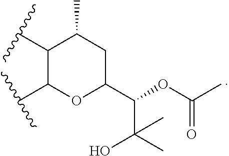 [SMILES: *C1OC([C@H](OC(C)=O)C(C)(C)O)C[C@@H](C)C1* ; "."]

In certain embodiments, Ring E is of either of the following formulae:

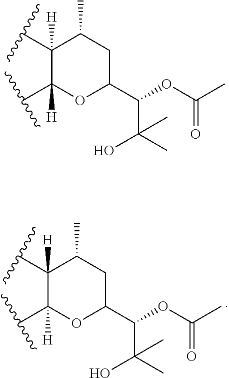 [SMILES: *[C@H]1[C@H](C)CC([C@H](OC(C)=O)C(C)(C)O)O[C@@H]1* ; *[C@@H]1[C@H](C)CC([C@H](OC(C)=O)C(C)(C)O)O[C@H]1* ; "."]

In some embodiments, Ring E is of any of the following formulae:

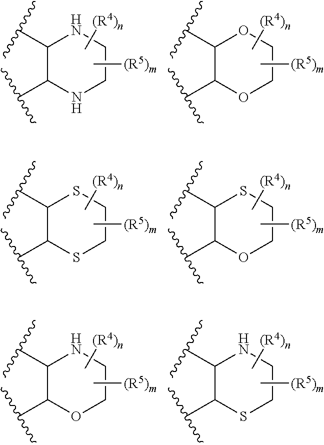

wherein each of R⁴, R⁵, m and n are as defined above and described herein. In certain embodiments wherein Ring E is of any one of the above formulae, isomeric forms are also contemplated. For example, it would be apparent to one of ordinary skill in the art that although 1,4-dioxane is described above, 1,3-dioxane and 1,2-dioxane are also contemplated herein.

In some embodiments, Ring E is a 5-7 membered partially unsaturated ring having 0-2 heteroatoms independently selected from nitrogen, oxygen, or sulfur. In certain embodiments, Ring E is a 5 membered partially unsaturated ring having 1-2 heteroatoms independently selected from nitrogen, oxygen, or sulfur. In certain embodiments, Ring E is a 5 membered partially unsaturated carbocycle. In certain embodiments, Ring E is a 6 membered partially unsaturated ring having 1-2 heteroatoms independently selected from nitrogen, oxygen, or sulfur. In certain embodiments, Ring E is a 6 membered partially unsaturated carbocycle. In certain embodiments, Ring E is a 7 membered partially unsaturated ring having 1-2 heteroatoms independently selected from nitrogen, oxygen, or sulfur. In certain embodiments, Ring E is a 7 membered partially unsaturated carbocycle.

Exemplary 5 membered partially unsaturated optionally substituted fused E rings include cyclopentene, dihydrofuran, dihydropyrrole, dihydrothiophene, dihydroimidazole, dihydrothiozole, and dihydrooxaaole. Exemplary 6 membered partially unsaturated optionally substituted E rings include cyclohexene, tetrahydropyrazine, dihydrooxazine, dihydrothiazine, dihydrodioxine, dihydrooxathiine, dihydropyran, tetrahydropyridine, dihydrothiopyran, and dihydrodithiine. Exemplary 7 membered partially unsaturated optionally substituted E rings include tetrahydrooxepine, dihydrooxepine, tetrahydroazepine, dihydroazepine, tetrahydrothiepine, and dihydrothiepine.

In some embodiments, a the present invention provides a compound of the formula V-h:

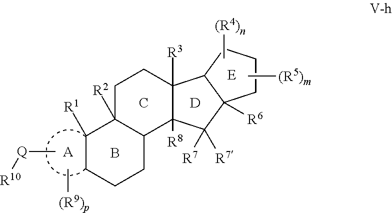

or a pharmaceutically acceptable salt thereof, wherein each variable is defined above and in classes and subclasses herein.

In some embodiments, Ring E is of any of the following formulae:

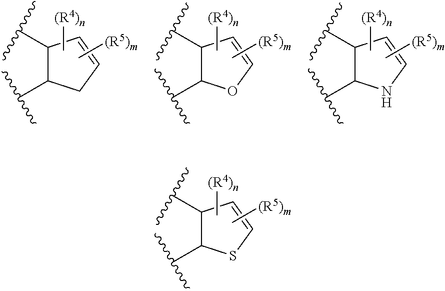

wherein each of R⁴, R⁵, n, and m are as defined above and described herein.

In some embodiments, Ring E is of any of the following formulae:

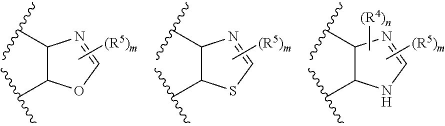

wherein each of R⁴, R⁵, n, and m are as defined above and described herein.

In some embodiments, Ring E is of any of the following formulae:

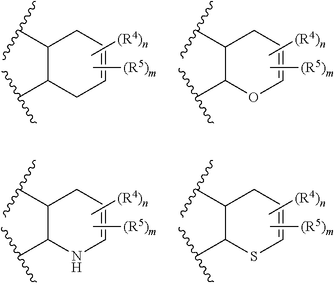

wherein R⁴, R⁵, n, and m are as defined above and described herein.

In some embodiments, Ring E is a 5-6 membered aromatic ring having 0-2 heteroatoms independently selected from nitrogen, oxygen, or sulfur. In certain embodiments, Ring E is a 5 membered aromatic ring having 1-2 heteroatoms independently selected from nitrogen, oxygen, or sulfur. In some embodiments, Ring E is a 6 membered aromatic ring having 1-2 heteroatoms independently selected from nitrogen, oxygen, or sulfur. In some embodiments, Ring E is benzo.

Exemplary 5 membered aromatic E rings include fused furano, pyrrolo, thiopheno, oxazolo, thiazolo, and imidazolo. Exemplary 6 membered aromatic E rings include benzo, pyridino, pyrimidino, triazino, and tetrazino.

In some embodiments, Ring E is of any of the following formulae:

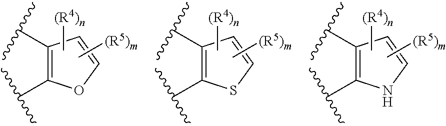

wherein each of R⁴, R⁵, n, and m are as defined above and described herein.

In some embodiments, Ring E is of any of the following formulae:

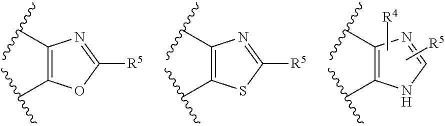

wherein each of R⁴ and R⁵ are as defined above and described herein.

In some embodiments, Ring E is of any of the following formulae:

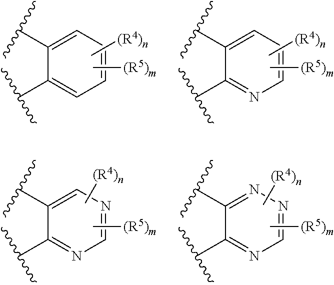

wherein each of R⁴, R⁵, n, and m are as defined above and described herein.

In some embodiments, the compound is of any one of the following formulae:

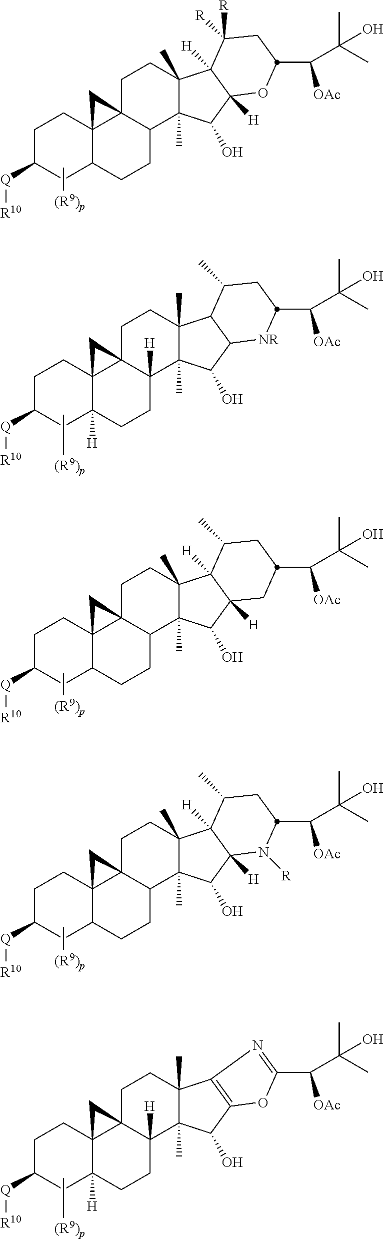

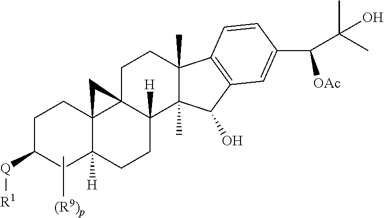

wherein each of R, R⁹, R¹⁰, and p are as defined above and described herein.

In certain embodiments, each R⁴ is independently selected from halogen, R, OR, or a suitably protected hydroxyl group. In certain embodiments, each R⁴ is independently selected from SR, a suitably protected thiol group, S(O)R, SO₂R, or OSO₂R. In certain embodiments, each R⁴ is independently selected from N(R)₂, a suitably protected amino group, N(R)C(O)R, N(R)C(O)C(O)R, N(R)C(O)N(R)₂, or N(R)C(O)OR. In certain embodiments, each R⁴ is independently selected from C(O)OR, OC(O)R, C(O)N(R)₂, or OC(O)N(R)₂. In certain embodiments, one or more R⁴ is independently R. In certain embodiments, one or more R⁴ is independently fluorine, methyl, or trifluoromethyl.

In some embodiments, two R⁴ on the same carbon are taken together to form an optionally substituted 3-8 membered spirofused ring having 0-4 heteroatoms independently selected from nitrogen, oxygen, or sulfur. In some embodiments, two R⁴ on the same carbon are taken together to form an optionally substituted 5-6 membered saturated spirofused ring having 0-2 heteroatoms independently selected from nitrogen, oxygen, or sulfur. In some embodiments, two R⁴ on the same carbon are taken together to form an optionally substituted 3-8 membered partially unsaturated spirofused ring having 0-4 heteroatoms independently selected from nitrogen, oxygen, or sulfur. In some embodiments, two R⁴ on the same carbon are taken together to form an optionally substituted 5-6 membered partially unsaturated spirofused ring having 0-2 heteroatoms independently selected from nitrogen, oxygen, or sulfur. In some embodiments, two R⁴ on the same carbon are taken together to form an oxo moiety. In some embodiments, two R⁴ on the same carbon are taken together to form an oxime. In some embodiments, two R⁴ on the same carbon are taken together to form a substituted hydrazone or substituted imine. In some embodiments, two R⁴ on the same carbon are taken together to form a unsubstituted hydrazone or unsubstituted imine. In some embodiments, two R⁴ on the same carbon are taken together to form an optionally substituted C₂₋₆ alkylidene. In some embodiments, two R⁴ on the same carbon are taken together to form an unsubstituted C₂ alkylidene. In some embodiments, two R⁴ on the same carbon are taken together to form a substituted C₂ alkylidene. In some embodiments, two R⁴ on the same carbon are taken together to form an unsubstituted C₃ alkylidene. In some embodiments, two R⁴ on the same carbon are taken together to form a substituted C₃ alkylidene. In some embodiments, two R⁴ on the same carbon are taken together to form an unsubstituted C₄ alkylidene. In some embodiments, two R⁴ on the same carbon are taken together to form a substituted C₄ alkylidene. In some embodiments, two R⁴ on the same carbon are taken together to form an unsubstituted C₅ alkylidene. In some embodiments, two R⁴ on the same carbon are taken together to form a substituted C₅ alkylidene. In some embodiments, two R⁴ on the same carbon are taken together to form an unsubstituted C₆ alkylidene. In some embodiments, two R⁴ on the same carbon are taken together to form a substituted C₆ alkylidene.

### 11. R⁵ Embodiments

As defined generally above and herein, each R⁵ is independently T-C(R′)₃, T-C(R′)₂C(R″)₃, OR, a suitably protected hydroxyl group, SR, a suitably protected thiol group, S(O)R, SO₂R, OSO₂R, N(R)₂, a suitably protected amino group, N(R)C(O)R, N(R)C(O)C(O)R, N(R)C(O)N(R)₂, N(R)C(O)OR, C(O)OR, OC(O)R, C(O)N(R)₂, or OC(O)N(R)₂, an optionally substituted 3-8 membered saturated, partially unsaturated, or aryl monocyclic ring having 0-4 heteroatoms independently selected from nitrogen, oxygen, or sulfur, an optionally substituted 8-10 membered saturated, partially unsaturated, or aryl bicyclic ring having 0-4 heteroatoms independently selected from nitrogen, oxygen, or sulfur, or:
two R⁵ on the same carbon are optionally taken together to form an oxo moiety, an oxime, an optionally substituted hydrazone, an optionally substituted imine, an optionally substituted C₂₋₆ alkylidene, or an optionally substituted 3-8 membered saturated or partially unsaturated spirocycle having 0-4 heteroatoms independently selected from nitrogen, oxygen, or sulfur;
each T is independently a valence bond or an optionally substituted straight or branched, saturated or unsaturated, C₁₋₆ alkylene chain wherein up to two methylene units of T are optionally and independently replaced by —O—, —N(R)—, —S—, —C(O)—, —S(O)—, or —S(O)₂—;
each R′ and R″ is independently selected from halogen, R, OR, SR, S(O)R, SO₂R, OSO₂R, N(R)₂, N(R)C(O)R, N(R)C(O)C(O)R, N(R)C(O)N(R)₂, N(R)C(O)OR, N(R)S(O)R, N(R)SO₂R, N(R)SO₂OR C(O)OR, OC(O)R, C(O)N(R)₂, OC(O)N(R)₂, or an optionally substituted 3-8 membered saturated, partially unsaturated, or aryl monocyclic ring having 0-4 heteroatoms independently selected from nitrogen, oxygen, or sulfur, or an optionally substituted 8-10 membered saturated, partially unsaturated, or aryl bicyclic ring having 0-4 heteroatoms independently selected from nitrogen, oxygen, or sulfur, or:
two R′ are optionally taken together to form an oxo moiety, an oxime, an optionally substituted hydrazone, an optionally substituted imine, an optionally substituted C₂₋₆ alkylidene, or an optionally substituted 3-8 membered saturated or partially unsaturated ring having 0-4 heteroatoms independently selected from nitrogen, oxygen, or sulfur, or:
two R″ are optionally taken together to form an oxo moiety, an oxime, an optionally substituted hydrazone, an optionally substituted imine, an optionally substituted C₂₋₆ alkylidene, or an optionally substituted 3-8 membered saturated or partially unsaturated ring having 0-4 heteroatoms independently selected from nitrogen, oxygen, or sulfur.

In certain embodiments, R⁵ is an optionally substituted 3-8 membered saturated monocyclic ring having 1-3 heteroatoms independently selected from nitrogen, oxygen, or sulfur. In certain embodiments, R⁵ is an optionally substituted 3-8 membered saturated monocyclic carbocycle. In certain embodiments, R⁵ is an optionally substituted 5-6 membered saturated monocyclic ring having 1-2 heteroatoms independently selected from nitrogen, oxygen, or sulfur. In certain embodiments, R⁵ is an optionally substituted 5-6 membered saturated monocyclic carbocycle.

Exemplary R⁵ saturated 3-8 membered optionally substituted heterocycles include oxirane, oxetane, tetrahydrofuran, tetrahydropyran, oxepane, aziridine, azetidine, pyrrolidine, piperidine, azepane, thiirane, thietane, tetrahydrothiophene, tetrahydrothiopyran, thiepane, dioxolane, oxathiolane, oxazolidine, imidazolidine, thiazolidine, dithiolane, dioxane, morpholine, oxathiane, piperazine, thiomorpholine, dithiane, dioxepane, oxazepane, oxathiepane, dithiepane, diazepane, dihydrofuranone, tetrahydropyranone, oxepanone, pyrrolidinone, piperidinone, azepanone, dihydrothiophenone, tetrahydrothiopyranone, thiepanone, oxazolidinone, oxazinanone, oxazepanone, dioxolanone, dioxanone, dioxepanone, oxathiolinone, oxathianone, oxathiepanone, thiazolidinone, thiazinanone, thiazepanone, imidazolidinone, tetrahydropyrimidinone, diazepanone, imidazolidinedione, oxazolidinedione, thiazolidinedione, dioxolanedione, oxathiolanedione, piperazinedione, morpholinedione, and thiomorpholinedione.

In certain embodiments, R⁵ is an optionally substituted 3-8 membered partially unsaturated monocyclic ring having 1-3 heteroatoms independently selected from nitrogen, oxygen, or sulfur. In certain embodiments, R⁵ is an optionally substituted 3-8 membered partially unsaturated monocyclic carbocycle. In certain embodiments, R⁵ is an optionally substituted 5-6 membered partially unsaturated monocyclic ring having 1-2 heteroatoms independently selected from nitrogen, oxygen, or sulfur. In certain embodiments, R⁵ is an optionally substituted 5-6 membered partially unsaturated monocyclic carbocycle. In certain embodiments, R⁵ is an optionally substituted 5-6 membered aryl ring having 0-3 heteroatoms independently selected from nitrogen, oxygen, or sulfur. In certain embodiments, R⁵ is an optionally substituted 5 membered aryl ring having 1-3 heteroatoms independently selected from nitrogen, oxygen, or sulfur. In certain embodiments, R⁵ is an optionally substituted 6 membered aryl ring having 1-3 heteroatoms independently selected from nitrogen, oxygen, or sulfur. In certain embodiments, R⁵ is an optionally substituted phenyl.

Exemplary optionally substituted R⁵ partially unsaturated monocyclic heterocycles include dihydrofuran, dihydropyran, tetrahydrooxepine, dihydropyrrole, tetrahydropyridine, tetrahydroazepine, dihydrothiophene, dihydrothiopyran, tetrahydrothiepine, furanone, dihydropyranone, dihydrooxepinone, pyrrolone, dihydropyridinone, dihydroazepinone, thiophenone, dihydrothiopyranone, dihydrothiepinone, pyrrolidione, furandione, dihydrooxazole, dihydrothiazole, oxathiole, oxathiine, dihydrooxazine, dihydrothiazine, tetrahydropyrimidine, tetrahydrooxazepine, tetrahydrothiazepine, and tetrahydrodiazepine.

In certain embodiments, R⁵ is an optionally substituted 8-10 membered saturated bicyclic ring having 0-4 heteroatoms independently selected from nitrogen, oxygen, or sulfur. In certain embodiments, R⁵ is an optionally substituted 8 membered saturated bicyclic ring having 1-3 heteroatoms independently selected from nitrogen, oxygen, or sulfur. In certain embodiments, R⁵ is an optionally substituted 8 membered saturated bicyclic carbocycle. In certain embodiments, R⁵ is an optionally substituted 9 membered saturated bicyclic ring having 1-3 heteroatoms independently selected from nitrogen, oxygen, or sulfur. In certain embodiments, R⁵ is an optionally substituted 9 membered saturated bicyclic carbocycle. In certain embodiments, R⁵ is an optionally substituted 10 membered saturated bicyclic ring having 1-3 heteroatoms independently selected from nitrogen, oxygen, or sulfur. In certain embodiments, R⁵ is an optionally substituted 10 membered saturated bicyclic carbocycle.

In certain embodiments, R⁵ is an optionally substituted 8-10 membered partially unsaturated bicyclic ring having 0-4 heteroatoms independently selected from nitrogen, oxygen, or sulfur. In certain embodiments, R⁵ is an optionally substituted 8 membered partially unsaturated bicyclic ring having 1-3 heteroatoms independently selected from nitrogen, oxygen, or sulfur. In certain embodiments, R⁵ is an optionally substituted 8 membered partially unsaturated bicyclic carbocycle. In certain embodiments, R⁵ is an optionally substituted 9 membered partially unsaturated bicyclic ring having 1-3 heteroatoms independently selected from nitrogen, oxygen, or sulfur. In certain embodiments, R⁵ is an optionally substituted 9 membered partially unsaturated bicyclic carbocycle. In certain embodiments, R⁵ is an optionally substituted 10 membered partially unsaturated bicyclic ring having 1-3 heteroatoms independently selected from nitrogen, oxygen, or sulfur. In certain embodiments, R⁵ is an optionally substituted 10 membered partially unsaturated bicyclic carbocycle.

In certain embodiments, R⁵ is an optionally substituted 9-10 membered aryl bicyclic ring having 0-4 heteroatoms independently selected from nitrogen, oxygen, or sulfur. In certain embodiments, R⁵ is an optionally substituted 9 membered aryl bicyclic ring having 1-4 heteroatoms independently selected from nitrogen, oxygen, or sulfur. In certain embodiments, R⁵ is an optionally substituted 9 membered aryl bicyclic ring having 3 heteroatoms independently selected from nitrogen, oxygen, or sulfur. In certain embodiments, R⁵ is an optionally substituted 9 membered aryl bicyclic ring having 2 heteroatoms independently selected from nitrogen, oxygen, or sulfur. In certain embodiments, R⁵ is an optionally substituted 9 membered aryl bicyclic ring having 1 heteroatom selected from nitrogen, oxygen, or sulfur. In certain embodiments, R⁵ is an optionally substituted 10 membered aryl bicyclic ring having 0-3 heteroatoms independently selected from nitrogen, oxygen, or sulfur. In certain embodiments, R⁵ is an optionally substituted 10 membered aryl bicyclic ring having 1-2 heteroatoms independently selected from nitrogen, oxygen, or sulfur. In certain embodiments, R⁵ is an optionally substituted naphthyl.

Exemplary optionally substituted R⁵ heteroaryl groups include thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, pteridinyl, indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, pyrido[2,3-b]-1,4-oxazin-3(4H)-one, or chromanyl.

In some embodiments, two R′ are optionally taken together to form an oxo moiety, an oxime, an optionally substituted hydrazone, an optionally substituted imine, or an optionally substituted C₂₋₆ alkylidene. In some embodiments, two R″ are optionally taken together to form an oxo moiety, an oxime, an optionally substituted hydrazone, an optionally substituted imine, or an optionally substituted C₂₋₆ alkylidene.

In some embodiments, two R⁵ on the same carbon are taken together to form an optionally substituted 3-8 membered saturated spirocycle having 0-2 heteroatoms independently selected from nitrogen, oxygen, or sulfur. In certain embodiments, two R⁵ on the same carbon are taken together to form an optionally substituted 3-6 membered saturated spirocycle having 0-2 heteroatoms independently selected from nitrogen, oxygen, or sulfur. In certain embodiments, two R⁵ on the same carbon are taken together to form an optionally substituted 3 membered saturated spirocycle having 0-1 heteroatom independently selected from nitrogen, oxygen, or sulfur.

In some embodiments, two R⁵ on the same carbon are taken together to form an optionally substituted 3-8 membered partially unsaturated spirocycle having 0-2 heteroatoms independently selected from nitrogen, oxygen, or sulfur. In certain embodiments, two R⁵ on the same carbon are taken together to form an optionally substituted 3-6 membered partially unsaturated spirocycle having 0-2 heteroatoms independently selected from nitrogen, oxygen, or sulfur. In certain embodiments, two R⁵ on the same carbon are taken together to form an optionally substituted 3 membered partially unsaturated spirocycle having 0-1 heteroatom independently selected from nitrogen, oxygen, or sulfur.

In some embodiments, two R⁵ on the same carbon are optionally taken together to form an oxo moiety. In some embodiments, two R⁵ on the same carbon are optionally taken together to form an oxime. In some embodiments, two R⁵ on the same carbon are optionally taken together to form a substituted hydrazone or substituted imine. In some embodiments, two R⁵ on the same carbon are optionally taken together to form an unsubstituted hydrazone or an unsubstituted imine.

In some embodiments, R⁵ and R⁶ are taken together to form an optionally substituted 3-8 membered saturated ring having 0-4 heteroatoms independently selected from nitrogen, oxygen, or sulfur. In some embodiments, R⁵ and R⁶ are taken together to form an optionally substituted 3-8 membered saturated ring having 1-3 heteroatoms independently selected from nitrogen, oxygen, or sulfur. In some embodiments, R⁵ and R⁶ are taken together to form an optionally substituted 5-6 membered saturated ring having 1-2 heteroatoms independently selected from nitrogen, oxygen, or sulfur.

In some embodiments, R⁵ and R⁶ are taken together to form an optionally substituted 3-8 membered partially unsaturated ring having 0-4 heteroatoms independently selected from nitrogen, oxygen, or sulfur. In some embodiments, R⁵ and R⁶ are taken together to form an optionally substituted 3-8 membered partially unsaturated ring having 1-3 heteroatoms independently selected from nitrogen, oxygen, or sulfur. In some embodiments, R⁵ and R⁶ are taken together to form an optionally substituted 5-6 membered partially unsaturated ring having 1-2 heteroatoms independently selected from nitrogen, oxygen, or sulfur.

In some embodiments, R⁵ and R⁶ are taken together to form an optionally substituted 3-8 membered aryl ring having 0-4 heteroatoms independently selected from nitrogen, oxygen, or sulfur. In some embodiments, R⁵ and R⁶ are taken together to form an optionally substituted 3-8 membered aryl ring having 1-3 heteroatoms independently selected from nitrogen, oxygen, or sulfur. In some embodiments, R⁵ and R⁶ are taken together to form an optionally substituted 5-6 membered aryl ring having 1-2 heteroatoms independently selected from nitrogen, oxygen, or sulfur.

In certain embodiments, when the R⁵ group of formula I is T-C(R′)₃ or T-C(R′)₂C(R″)₃, each T is independently a valence bond or a straight or branched C₁₋₄ alkylene chain wherein one methylene unit of T is optionally replaced by —O—, —N(R)—, or —S—. In other embodiments, each T is independently a valence bond or a straight or branched C₁₋₄ alkylene chain. In still other embodiments, each T is a valence bond.

In certain embodiments, as described generally above, when the R⁵ group of formula I is T-C(R′)₃ or T-C(R′)₂C(R″)₃, each R′ and R″ is independently selected from halogen, R, OR, SR, S(O)R, SO₂R, OSO₂R, N(R)₂, N(R)C(O)R, N(R)C(O)(CO)R, N(R)C(O)N(R)₂, N(R)C(O)OR, N(R)S(O)R, N(R)SO₂R, N(R)SO₂OR C(O)OR, OC(O)R, C(O)N(R)₂, or OC(O)N(R)₂. In certain embodiments, each R′ and R″ is independently selected from halogen, R, OR, SR, N(R)₂, N(R)C(O)R, N(R)C(O)N(R)₂, N(R)SO₂R, N(R)C(O)OR, C(O)OR, OC(O)R, C(O)N(R)₂, or OC(O)N(R)₂. In certain embodiments, each R′ and R″ is independently halogen, R, OR, OC(O)R, SR, or N(R)₂. In other embodiments, each R′ and R″ is independently halogen, R, OR, or OC(O)R.

In certain embodiments, one or more occurrence of R′ is independently an aliphatic group optionally substituted with one or more halo substituents. In certain embodiments, one or more occurrence of R′ is independently optionally substituted with one or more fluorine substituents. In certain embodiments, one or more occurrence of R′ is independently haloalkyl.

In certain embodiments, one or more occurrence of R″ is independently an aliphatic group optionally substituted with one or more halo substituents. In certain embodiments, one or more occurrence of R″ is independently optionally substituted with one or more fluorine substituents. In certain embodiments, one or more occurrence of R″ is independently haloalkyl.

In certain embodiments, the R⁵ group of formula I is T-CF(R′)₂, T-CF₂(R′), T-C(R′)₂C(R″)₃, T-CF(R′)C(R″)₃, T-CF(R′)CF(R″)₂, T-CF(R′)CF₂(R″), T-CF(R′)CF₃, T-CF₂C(R″)₃, T-CF₂CF(R″)₂, T-CF₂CF₂(R″), or T-CF₂CF₃.

In certain embodiments, T is a valence bond and one or more R′ is independently fluorine. In certain embodiments, T is a valence bond and one or more R′ is independently a C₁₋₆ aliphatic group optionally substituted with fluorine. In certain embodiments, T is a valence bond and one or more R′ is independently OC(O)R, wherein R is an aliphatic group optionally substituted with fluorine.

In certain embodiments, as defined generally above and herein, when the R⁵ group of formula I is T-C(R′)₃ or T-C(R′)₂C(R″)₃, one or more R′ or R″ is independently selected from an optionally substituted 3-8 membered saturated, partially unsaturated, or aryl monocyclic ring having 0-4 heteroatoms independently selected from nitrogen, oxygen, or sulfur, or an optionally substituted 8-10 membered saturated, partially unsaturated, or aryl bicyclic ring having 0-4 heteroatoms independently selected from nitrogen, oxygen, or sulfur. In certain embodiments, one or more of R′ or R″ is independently an optionally substituted 3-8 membered saturated monocyclic ring having 0-3 heteroatoms independently selected from nitrogen, oxygen, or sulfur. In certain embodiments, one or more of R′ or R″ is independently an optionally substituted 3-6 membered saturated monocyclic ring having 1-2 heteroatoms independently selected from nitrogen, oxygen, or sulfur. In certain embodiments, one or more of R′ or R″ is independently an optionally substituted 3-6 membered saturated monocyclic carbocycle. In certain embodiments, one or more of R′ or R″ is independently cyclopropyl, cyclobutyl, cyclopentyl, or cyclohexyl.

As defined generally above and herein, in certain embodiments, two R′ are optionally taken together to form an optionally substituted 3-8 membered saturated or partially unsaturated ring having 0-4 heteroatoms independently selected from nitrogen, oxygen, or sulfur. In certain embodiments, two R′ are optionally taken together to form an optionally substituted 3-6 membered saturated ring having 1-4 heteroatoms independently selected from nitrogen, oxygen, or sulfur. In certain embodiments, two R′ are optionally taken together to form an optionally substituted 3-6 membered saturated carbocycle. In certain embodiments, two R′ are optionally taken together to form an optionally substituted 3 membered saturated carbocycle. In certain embodiments, two R′ are optionally taken together to form an optionally substituted 5-8 membered partially unsaturated ring having 1-4 heteroatoms independently selected from nitrogen, oxygen, or sulfur. In certain embodiments, two R′ are optionally taken together to form an optionally substituted 5-8 membered partially unsaturated carbocycle.

As defined generally above and herein, in certain embodiments, two R″ are optionally taken together to form an optionally substituted 3-8 membered saturated or partially unsaturated ring having 0-4 heteroatoms independently selected from nitrogen, oxygen, or sulfur. In certain embodiments, two R″ are optionally taken together to form an optionally substituted 3-6 membered saturated ring having 1-4 heteroatoms independently selected from nitrogen, oxygen, or sulfur. In certain embodiments, two R″ are optionally taken together to form an optionally substituted 3-6 membered saturated carbocycle. In certain embodiments, two R″ are optionally taken together to form an optionally substituted 3 membered saturated carbocycle. In certain embodiments, two R″ are optionally taken together to form an optionally substituted 5-8 membered partially unsaturated ring having 1-4 heteroatoms independently selected from nitrogen, oxygen, or sulfur. In certain embodiments, two R″ are optionally taken together to form an optionally substituted 5-8 membered partially unsaturated carbocycle.

Exemplary optionally substituted R′ and R″ saturated monocyclic heterocycles include oxirane, oxetane, tetrahydrofuran, tetrahydropyran, oxepane, aziridine, azetidine, pyrrolidine, piperidin, azepanes, thiiranes, thietane, tetrahydrothiophene, tetrahydrothiopyran, thiepane, dioxolane, oxathiolane, oxazolidine, imidazolidine, thiazolidine, dithiolane, dioxanes, morpholine, oxathiane, piperazine, thiomorpholine, dithiane, dioxepane, oxazepane, oxathiepane, dithiepane, diazepane, dihydrofuranone, tetrahydropyranone, oxepanone, pyrrolidinone, piperidinone, azepanone, dihydrothiophenone, tetrahydrothiopyranone, thiepanone, oxazolidinone, oxazinanone, oxazepanone, dioxolanone, dioxanone, dioxepanone, oxathiolinone, oxathianone, oxathiepanone, thiazolidinone, thiazinanone, thiazepanone, imidazolidinone, tetrahydropyrimidinone, diazepanone, imidazolidinedione, oxazolidinedione, thiazolidinedione, dioxolanedione, oxathiolanedione, piperazinedione, morpholinedione, and thiomorpholinedione.

In certain embodiments, one or more of R′ or R″ is independently an optionally substituted 3-8 membered partially unsaturated monocyclic ring having 0-3 heteroatoms independently selected from nitrogen, oxygen, or sulfur. In certain embodiments, one or more of R′ or R″ is independently an optionally substituted 5-6 membered partially unsaturated monocyclic ring having 1-2 heteroatoms independently selected from nitrogen, oxygen, or sulfur. In certain embodiments, one or more of R′ or R″ is independently an optionally substituted 5-6 membered partially unsaturated monocyclic carbocycle.

Exemplary optionally substituted R′ and R″ partially unsaturated monocyclic heterocycles include dihydrofuran, dihydropyran, tetrahydrooxepine, dihydropyrrole, tetrahydropyridine, tetrahydroazepine, dihydrothiophene, dihydrothiopyran, tetrahydrothiepine, furanone, dihydropyranone, dihydrooxepinone, pyrrolone, dihydropyridinone, dihydroazepinone, thiophenone, dihydrothiopyranone, dihydrothiepinone, pyrrolidione, furandione, dihydrooxazole, dihydrothiazole, oxathiole, oxathiine, dihydrooxazine, dihydrothiazine, tetrahydropyrimidine, tetrahydrooxazepine, tetrahydrothiazepine, and tetrahydrodiazepine.

In certain embodiments, one or more of R′ or R″ is independently an optionally substituted 5-6 membered aryl ring having 0-3 heteroatoms independently selected from nitrogen, oxygen, or sulfur. In certain embodiments, one or more of R′ or R″ is independently an optionally substituted 5 membered aryl ring having 1-3 heteroatoms independently selected from nitrogen, oxygen, or sulfur. In certain embodiments, one or more of R′ or R″ is independently an optionally substituted 6 membered aryl ring having 1-3 heteroatoms independently selected from nitrogen, oxygen, or sulfur. In certain embodiments, one or more of R′ or R″ is independently is an optionally substituted phenyl.

In certain embodiments, one or more of R′ or R″ is independently an optionally substituted 8-10 membered saturated bicyclic ring having 0-4 heteroatoms independently selected from nitrogen, oxygen, or sulfur. In certain embodiments, one or more of R′ or R″ is an optionally substituted 8 membered saturated bicyclic ring having 0-2 heteroatoms independently selected from nitrogen, oxygen, or sulfur. In certain embodiments, one or more of R′ or R″ is an optionally substituted 9 membered saturated bicyclic ring having 0-2 heteroatoms independently selected from nitrogen, oxygen, or sulfur. In certain embodiments, one or more of R′ or R″ is an optionally substituted 10 membered saturated bicyclic ring having 0-3 heteroatoms independently selected from nitrogen, oxygen, or sulfur.

In certain embodiments, one or more of R′ or R″ is an optionally substituted 8-10 membered partially unsaturated bicyclic ring having 0-4 heteroatoms independently selected from nitrogen, oxygen, or sulfur. In certain embodiments, one or more of R′ or R″ is an optionally substituted 8 membered partially unsaturated bicyclic ring having 0-3 heteroatoms independently selected from nitrogen, oxygen, or sulfur. In certain embodiments, one or more of R′ or R″ is an optionally substituted 9 membered partially unsaturated bicyclic ring having 0-3 heteroatoms independently selected from nitrogen, oxygen, or sulfur. In certain embodiments, one or more of R′ or R″ is an optionally substituted 10 membered partially unsaturated bicyclic ring having 0-3 heteroatoms independently selected from nitrogen, oxygen, or sulfur.

In certain embodiments, one or more of R′ or R″ is an optionally substituted 9-10 membered aryl bicyclic ring having 0-4 heteroatoms independently selected from nitrogen, oxygen, or sulfur. In certain embodiments, one or more of R′ or R″ is an optionally substituted 9 membered aryl bicyclic ring having 1-4 heteroatoms independently selected from nitrogen, oxygen, or sulfur. In certain embodiments, one or more of R′ or R″ is an optionally substituted 10 membered aryl bicyclic ring having 0-3 heteroatoms independently selected from nitrogen, oxygen, or sulfur. In certain embodiments, one or more of R′ or R″ is an optionally substituted 10 membered aryl bicyclic ring having 1-2 heteroatoms independently selected from nitrogen, oxygen, or sulfur. In certain embodiments, one or more of R′ or R″ is optionally substituted naphthyl.

Exemplary optionally substituted R′ or R″ heteroaryl groups include thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, pteridinyl, indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one, or chromanyl.

In some embodiments, T is an optionally substituted C₁₋₄ alkylene chain wherein one or more methylene units of T is independently replaced by —O—. In some embodiments, T is an optionally substituted C₁₋₄ alkylene chain wherein one or more methylene units of T is independently replaced by —C(O)—. In some embodiments, T is an optionally substituted C₂₋₄ alkylene chain wherein two methylene units of T are independently replaced by —O— and —C(O)—. In some embodiments, T is an optionally substituted C₂₋₄ alkylene chain wherein two methylene units of T are independently replaced by —O— and —S(O)—. In some embodiments, T is an optionally substituted C₂₋₄ alkylene chain wherein two methylene units of T are independently replaced by —O— and —S(O)₂—. In some embodiments, T is an optionally substituted C₁₋₄ alkylene chain wherein two methylene units of T are independently replaced by —O— and —C(O)— and wherein the one or more methylene unit is optionally substituted with fluorine. In some embodiments, T is an optionally substituted C₁₋₄ alkylene chain wherein two methylene units of T are independently replaced by —O— and —C(O)— and wherein one or more occurrence of R′ is independently OR. In some embodiments, T is an optionally substituted C₁₋₄ alkylene chain wherein two methylene units of T are independently replaced by —O— and —C(O)— and wherein one or more occurrence of R′ is fluorine. In some embodiments, T is an optionally substituted C₁₋₄ alkylene chain wherein two methylene units of T are independently replaced by —O— and —C(O)— and wherein one or more occurrence of R′ is independently optionally substituted C₁ aliphatic. In some embodiments, T is an optionally substituted C₁₋₄ alkylene chain wherein two methylene units of T are independently replaced by —O— and —C(O)— and wherein one or more occurrence of R′ is independently CF₃.

In some embodiments, T is a C₁₋₆ aliphatic group optionally substituted with one or more fluorine atoms. In some embodiments, T is a C₁₋₆ aliphatic group optionally substituted with one or more OR, wherein each occurrence of R is independently an optionally substituted C₁₋₆ aliphatic group. In certain embodiments, one or more occurrence of R is substituted with one or more fluorine moieties. By way of non-limiting example, exemplary OR groups include OCF₃, OCF₂H, OCFH₂, and OCF₂CF₃.

Exemplary R′ and R″ groups include hydrogen, F, CH₃, CF₃, CF₂H, CFH₂, CF₂CF₃, CF₂CHF₂, CF₂CH₂F, CF₂CH₃, CHFCH₃, CHFCH₂F, CHFCHF₂, CHFCF₃, OH, OCF₃, OCF₂H, OCFH₂, OCF₂CF₃, OCF₂CHF₂, OCF₂CH₂F, OCF₂CH₃, OCHFCH₃, OCHFCH₂F, OCHFCHF₂, OCHFCF₃, OC(O)CH₃, OC(O)CH₂CH₃, OC(O)CH(CH₃)₂, OC(O)CF₃, OC(O)CF₂H, OC(O)CFH₂, OC(O)CF₂CF₃, OC(O)CF₂CHF₂, OC(O)CF₂CH₂F, OC(O)CF₂CH₃, OC(O)CHFCH₃, OC(O)CHFCH₂F, OC(O)CHFCHF₂, OC(O)CHFCF₃, OC(O)CF(CH₃)₂, OC(O)CF(CF₃)₂, OC(O)CF(CF₃)(CF₂H), OC(O)CF(CF₃)(CFH₂), OC(O)CF(CF₃)(CH₃), OC(O)CF(CF₂H)(CH₃), and OC(O)CF(CFH₂)(CH₃).

As defined generally above, the R⁵ group of formula I is, inter alia, a suitably protected hydroxyl group, a suitably protected thiol group, or a suitably protected amino group. Hydroxyl protecting groups are well known in the art and include those described in detail in Protecting Groups in Organic Synthesis, T. W. Greene and P. G. M. Wuts, 3^{rd} edition, John Wiley & Sons, 1999, the entirety of which is incorporated herein by reference. Examples of suitably protected hydroxyl groups of the R⁵ group of formula I further include, but are not limited to, esters, allyl ethers, ethers, silyl ethers, alkyl ethers, arylalkyl ethers, and alkoxyalkyl ethers. Examples of such esters include formates, acetates, carbonates, and sulfonates. Specific examples include formate, benzoyl formate, chloroacetate, trifluoroacetate, methoxyacetate, triphenylmethoxyacetate, p-chlorophenoxyacetate, 3-phenylpropionate, 4-oxopentanoate, 4,4-(ethylenedithio)pentanoate, pivaloate (trimethylacetyl), crotonate, 4-methoxy-crotonate, benzoate, p-benzylbenzoate, 2,4,6-trimethylbenzoate, and carbonates such as methyl, 9-fluorenylmethyl, ethyl, 2,2,2-trichloroethyl, 2-(trimethylsilyl)ethyl, 2-(phenylsulfonyl)ethyl, vinyl, allyl, and p-nitrobenzyl. Examples of such silyl ethers include trimethylsilyl, triethylsilyl, t-butyldimethylsilyl, t-butyldiphenylsilyl, triisopropylsilyl, and other trialkylsilyl ethers. Alkyl ethers include methyl, benzyl, p-methoxybenzyl, 3,4-dimethoxybenzyl, trityl, t-butyl, allyl, and allyloxycarbonyl ethers or derivatives. Alkoxyalkyl ethers include acetals such as methoxymethyl, methylthiomethyl, (2-methoxyethoxy)methyl, benzyloxymethyl, beta-(trimethylsilyl)ethoxymethyl, and tetrahydropyranyl ethers. Examples of arylalkyl ethers include benzyl, p-methoxybenzyl (MPM), 3,4-dimethoxybenzyl, O-nitrobenzyl, p-nitrobenzyl, p-halobenzyl, 2,6-dichlorobenzyl, p-cyanobenzyl, and 2- and 4-picolyl.

Thiol protecting groups are well known in the art and include those described in detail in Protecting Groups in Organic Synthesis, T. W. Greene and P. G. M. Wuts, 3^{rd} edition, John Wiley & Sons, 1999, the entirety of which is incorporated herein by reference. Suitably protected thiol groups of the R⁵ moiety of formula I include, but are not limited to, disulfides, thioethers, silyl thioethers, thioesters, thiocarbonates, thiocarbamates, and the like. Examples of such groups include, but are not limited to, alkyl thioethers, benzyl and substituted benzyl thioethers, triphenylmethyl thioethers, trichloroethoxycarbonyl, to name but a few.

According to another aspect of the present invention, the R⁵ moiety of formula I is a thiol protecting group that is removable under neutral conditions e.g. with AgNO₃, HgCl₂, and the like. Other neutral conditions include reduction using a suitable reducing agent. Suitable reducing agents include dithiothreitol (DTT), mercaptoethanol, dithionite, reduced glutathione, reduced glutaredoxin, reduced thioredoxin, substituted phosphines such as tris carboxyethyl phosphine (TCEP), and any other peptide or organic based reducing agent, or other reagents known to those of ordinary skill in the art. According to yet another aspect of the present invention, the R⁵ moiety of formula I is a thiol protecting group that is “photocleavable”. Such suitable thiol protecting groups are known in the art and include, but are not limited to, a nitrobenzyl group, a tetrahydropyranyl (THP) group, a trityl group, —CH₂SCH₃ (MTM), dimethylmethoxymethyl, or —CH₂—S—S-pyridin-2-yl. One of ordinary skill in the art would recognize that many of the suitable hydroxyl protecting groups, as described herein, are also suitable as thiol protecting groups.

In certain embodiments, the R⁵ group of formula I is a suitably protected amino group. Amino protecting groups are well known in the art and include those described in detail in Protecting Groups in Organic Synthesis, T. W. Greene and P. G. M. Wuts, 3^{rd} edition, John Wiley & Sons, 1999, the entirety of which is incorporated herein by reference. Suitably protected amino groups of said R⁵ moiety further include, but are not limited to, aralkylamines, carbamates, cyclic imides, allyl amines, amides, and the like. Examples of such groups include t-butyloxycarbonyl (BOC), ethyloxycarbonyl, methyloxycarbonyl, trichloroethyloxycarbonyl, allyloxycarbonyl (Alloc), benzyloxocarbonyl (CBZ), allyl, phthalimide, benzyl (Bn), fluorenylmethylcarbonyl (Fmoc), formyl, acetyl, chloroacetyl, dichloroacetyl, trichloroacetyl, phenylacetyl, trifluoroacetyl, benzoyl, and the like. In certain embodiments, the amino protecting group of the R⁵ moiety is phthalimido. In still other embodiments, the amino protecting group of the R⁵ moiety is a tert-butyloxycarbonyl (BOC) group.

In some embodiments, R⁵ is of the following formula:

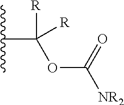

wherein R is as defined and descrined above and herein.

In some embodiments, R⁵ is of either of the following formulae:

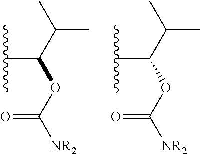

wherein R is as defined and descrined above and herein. In some embodiments, R⁵ is as depicted above, wherein two R on the same nitrogen atom of R⁵ are taken together with said nitrogen atom to form an optionally substituted 3-8 membered saturated, partially unsaturated, or aryl ring having 0-4 heteroatoms independently selected from nitrogen, oxygen, or sulfur. In some embodiments, R⁵ is as depicted above, wherein two R on the same nitrogen atom of R⁵ are taken together with said nitrogen atom to form an optionally substituted 4 membered saturated ring. In some embodiments, R⁵ is as depicted above, wherein each R of R⁵ is independently hydrogen or an optionally substituted C₁₋₆ aliphatic group. In certain embodiments, each R of R⁵ is methyl. In certain embodiments, one R of R⁵ is methyl and one R of R⁵ is hydrogen.

In some embodiments, R⁵ is of either of the following formulae:

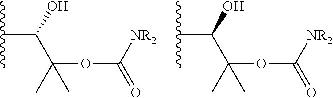

wherein each R is as defined and descrined above and herein.

In some embodiments, R⁵ is of either of the following formulae:

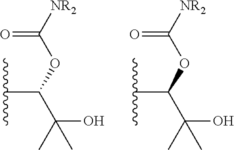

wherein each R is as defined and descrined above and herein.

In some embodiments, R⁵ is of either of the following formulae:

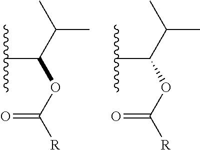

wherein each R is as defined and descrined above and herein.

In some embodiments, R⁵ is of either of the following formulae:

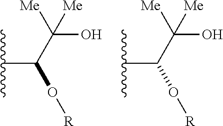

wherein each R is as defined and descrined above and herein.

In some embodiments, R⁵ is of any of the following formulae:

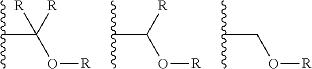

wherein R is as defined and descrined above and herein.

In some embodiments, R⁵ is of the following formula:

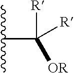

wherein each R is as defined and descrined above and herein, and wherein R′ are taken together to form a C₂₋₆ alkylidene moiety. In some embodiments, R⁵ is

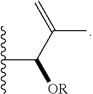

In some embodiments, R⁵ is of either of the following formulae:

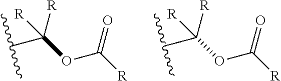

wherein each R is as defined and descrined above and herein.

In some embodiments, R⁵ is of either of the following formulae:

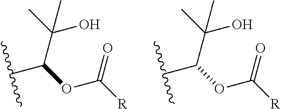

wherein each R is as defined and descrined above and herein.

In some embodiments, R⁵ is of either of the following formulae:

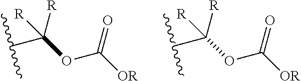

wherein each R is as defined and described above and herein.

Exemplary R⁵ groups are depicted below:

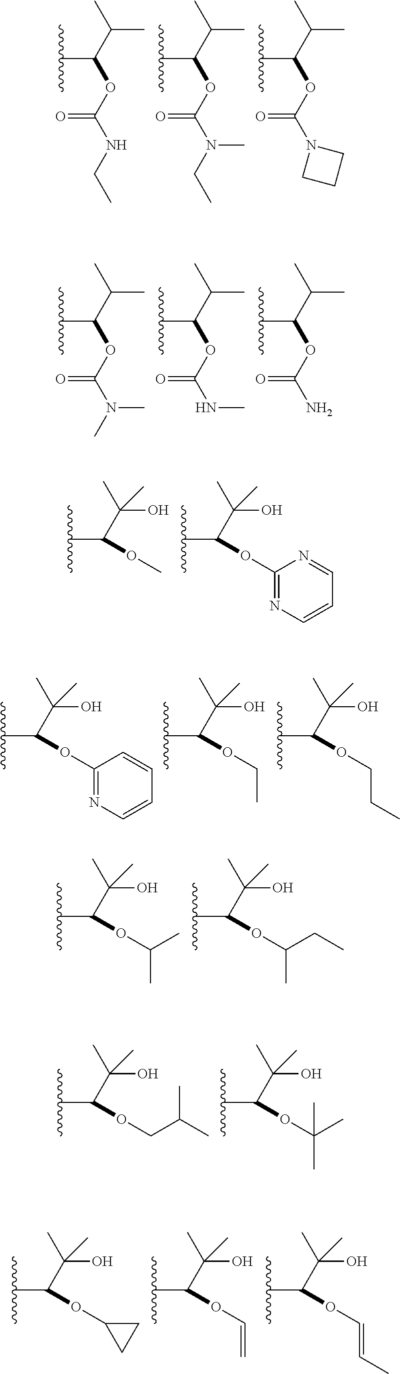

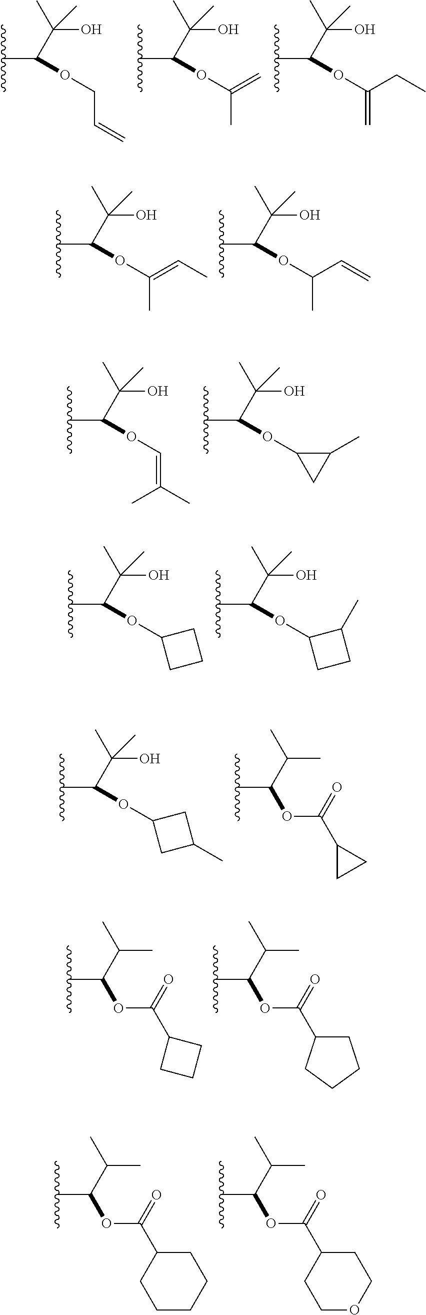

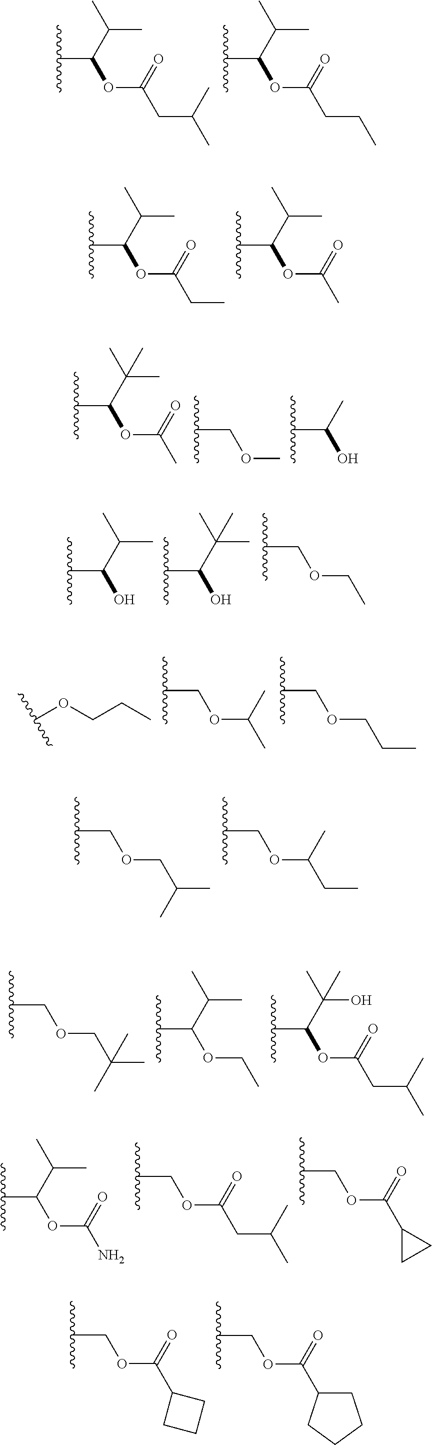

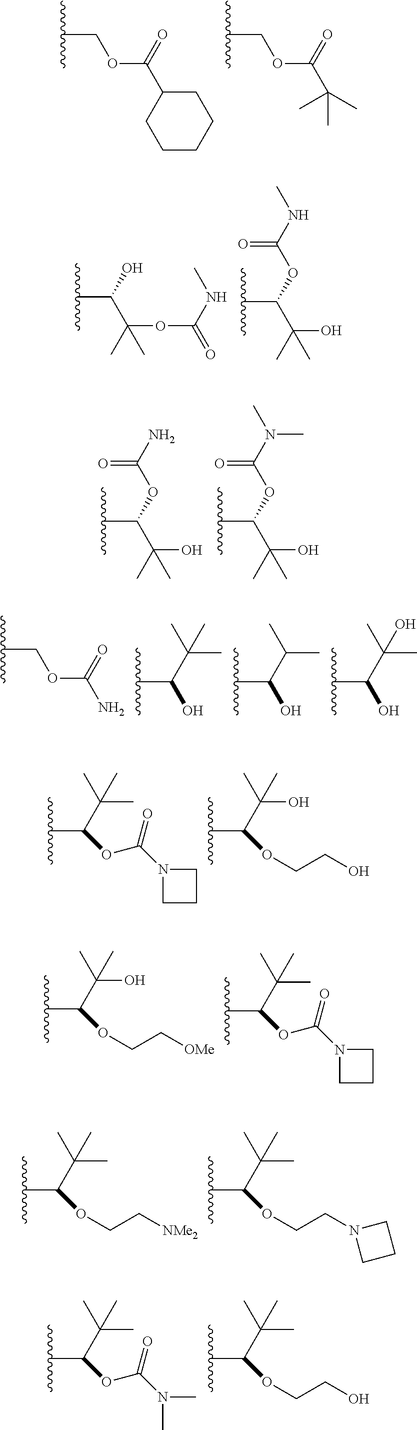

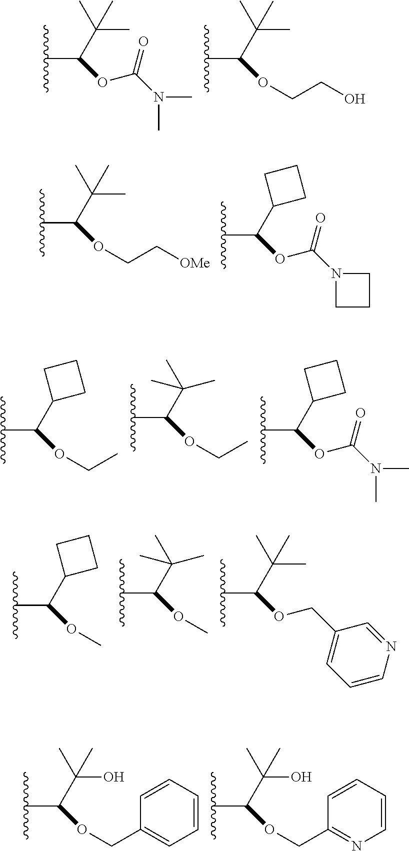

In some embodiments, wherein the present invention provides an R⁵ group containing one or more oxygen atoms, the present invention contemplates the independent replacement of the one or more oxygen atoms with one or more sulfur atoms. Such sulfur atoms may exist in any available oxidation state. For instance, in some embodiments, one or more —O— is independently replaced with —S—, —S(O)—, or —SO₂—. Exemplary such replacements are depicted below:

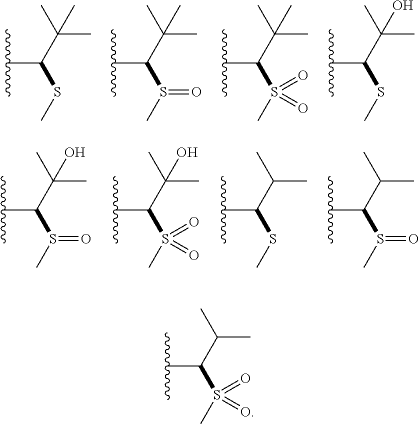

In some embodiments, R⁵ is of any of the following formulae:

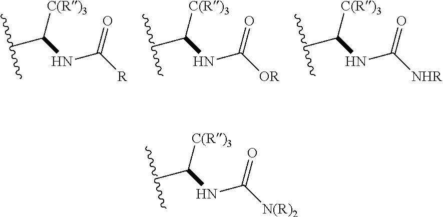

wherein each R″ is independently selected from halogen, R, OR, SR, S(O)R, SO₂R, OSO₂R, N(R)₂, N(R)C(O)R, N(R)C(O)C(O)R, N(R)C(O)N(R)₂, N(R)C(O)OR, N(R)S(O)R, N(R)SO₂R, N(R)SO₂OR C(O)OR, OC(O)R, C(O)N(R)₂, OC(O)N(R)₂, or an optionally substituted 3-8 membered saturated, partially unsaturated, or aryl monocyclic ring having 0-4 heteroatoms independently selected from nitrogen, oxygen, or sulfur, or an optionally substituted 8-10 membered saturated, partially unsaturated, or aryl bicyclic ring having 0-4 heteroatoms independently selected from nitrogen, oxygen, or sulfur, and wherein each R is as defined and described generally above and herein. In some embodiments, R⁵ is of any one of the formulae depicted above and each R″ is independently R, an optionally substituted 5-6 membered aryl ring having 0-4 heteroatoms independently selected from nitrogen, oxygen, or sulfur, or an optionally substituted 8-10 membered aryl bicyclic ring having 0-4 heteroatoms independently selected from nitrogen, oxygen, or sulfur.

Exemplary R⁵ groups are depicted below:

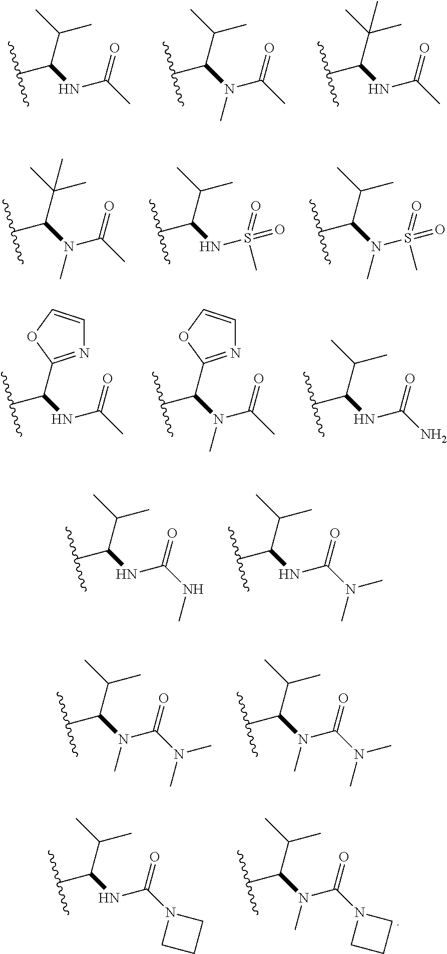

In some embodiments, R⁵ is of any of the following formulae:

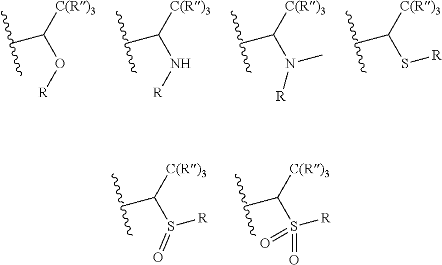

wherein each R and R″ is independently as defined and described above and herein.

In some embodiments, R⁵ is of any of the following formulae:

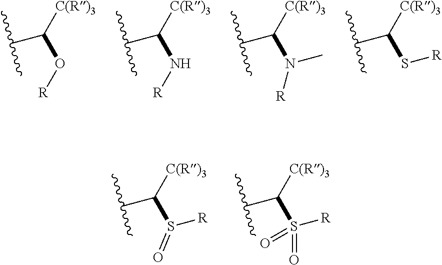

wherein each R″ is independently selected from R, an optionally substituted 3-8 membered saturated, partially unsaturated, or aryl monocyclic ring having 0-4 heteroatoms independently selected from nitrogen, oxygen, or sulfur, or an optionally substituted 8-10 membered saturated, partially unsaturated, or aryl bicyclic ring having 0-4 heteroatoms independently selected from nitrogen, oxygen, or sulfur, and wherein each R is as defined and described generally above and herein. In certain embodiments, R⁵ is of any one of the formulae depicted above and R″ is R. In certain embodiments, R⁵ is of any one of the formulae depicted above and R″ is a 5-6 membered aryl ring having 0-3 heteroatoms independently selected from nitrogen, oxygen, or sulfur.

Exemplary R⁵ groups are depicted below:

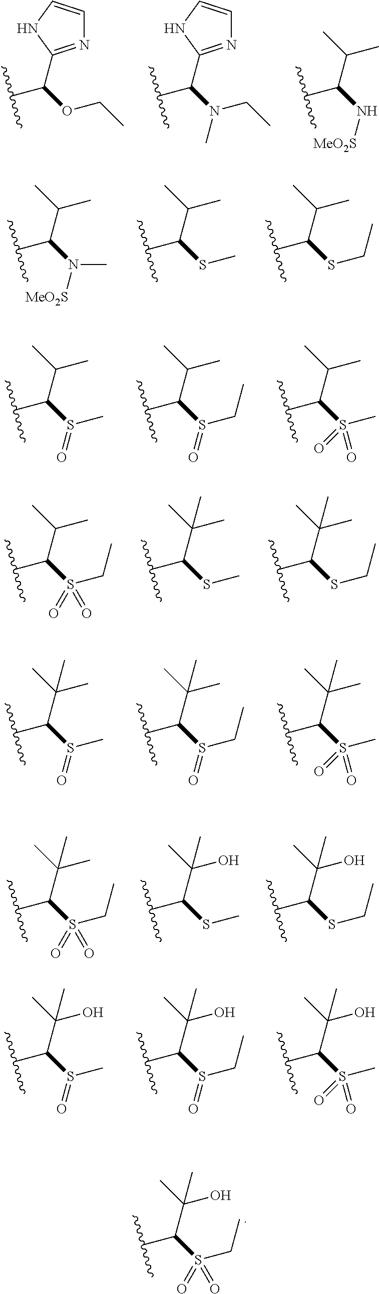

In certain embodiments, the compound is of any of the following formulae:

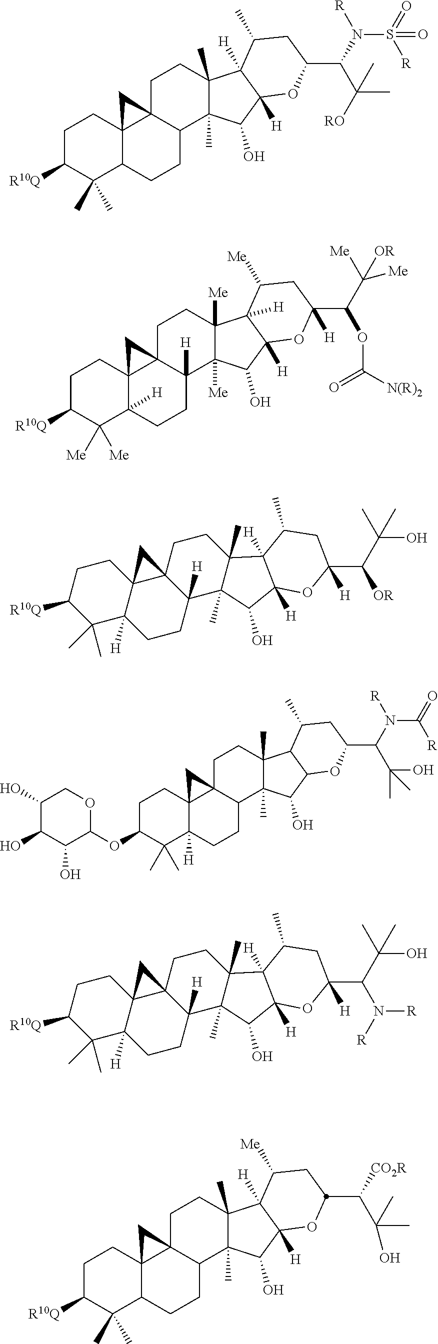

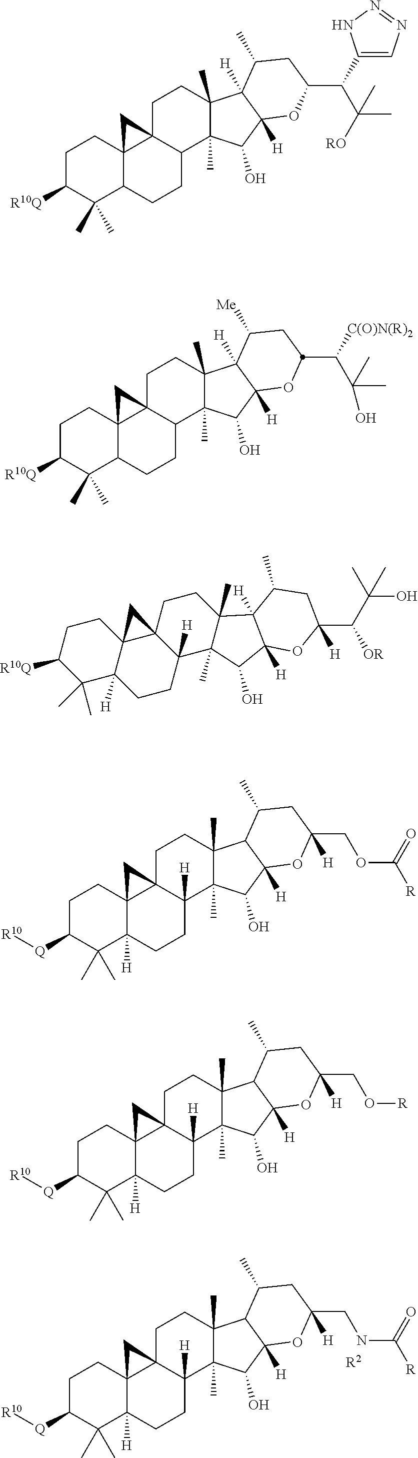

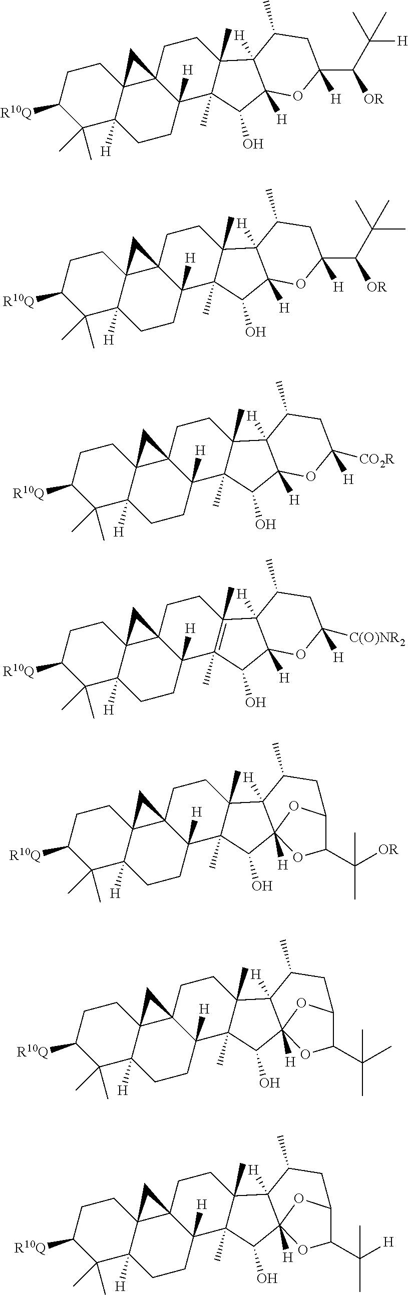

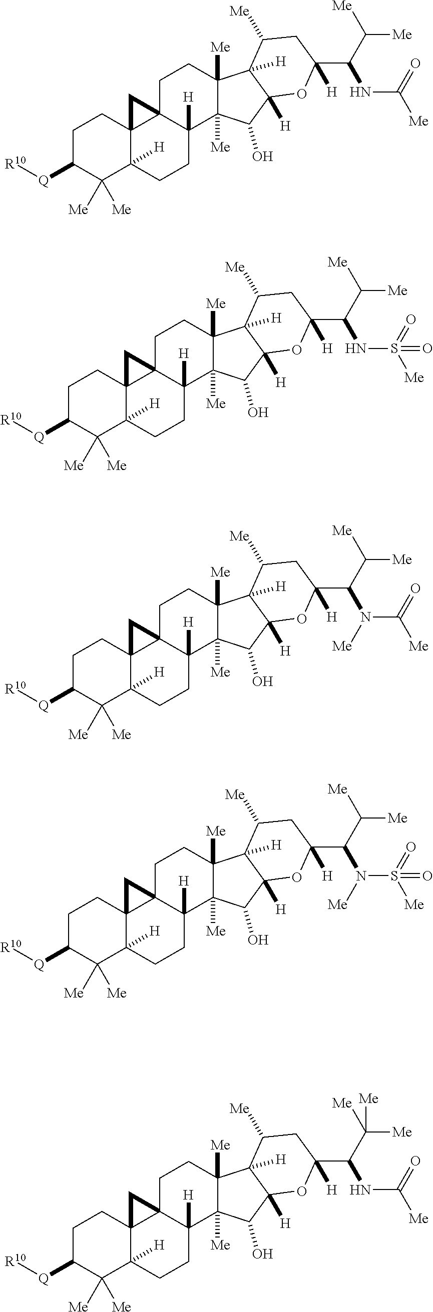

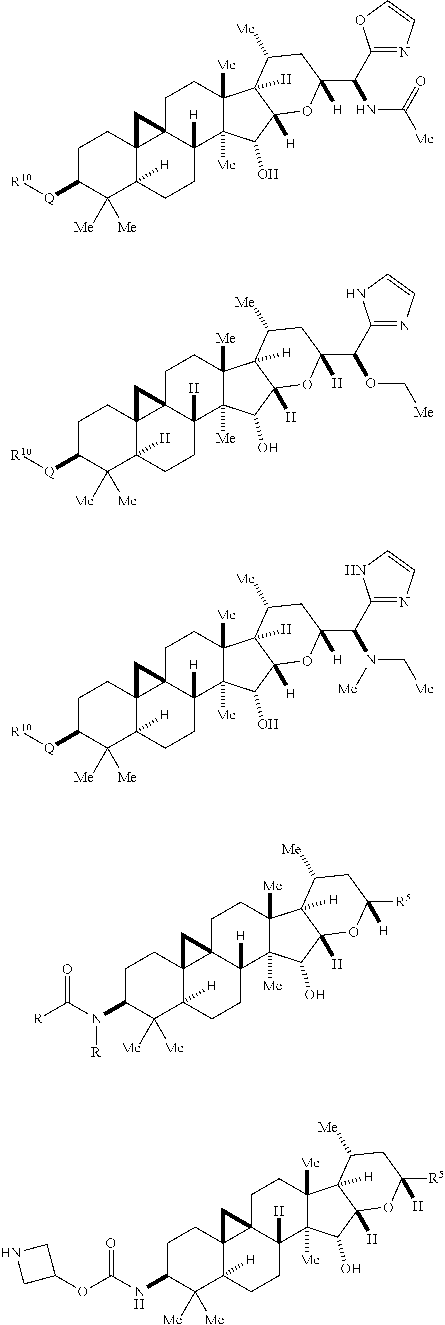

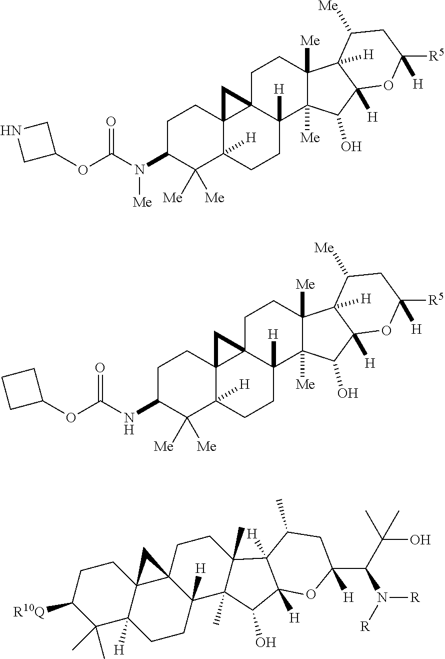

wherein R, R⁵, R¹⁰, and Q are as defined above and herein.

### 12. Exemplary Combinations

It will be appreciated that all combinations of embodiments, as described herein, are contemplated. In some embodiments, the present invention provides a compound having one or more of, or any combination of, the characteristics described below. It will further be appreciated that wherein a specific ring is described (e.g., Ring A, Ring B, Ring C, Ring D, and/or Ring E), the present invention additionally contemplates all embodiments of substituents on that ring. For instance, it will be appreciated that a description of Ring A of the present invention also contemplates all embodiments of R⁹, p, Q, R¹, and R¹⁰, unless otherwise specified.

One of skill in the art, based on the teachings herein, would understand how to make the following exemplary combinations and other embodiments described herein. In particular, one of skill in the art would recognize that numerous compounds of the present invention can be accessed via common synthetic intermediates described herein and that the scope of compounds described herein is therefore extensive. Exemplary such synthetic intermediates and reactions are depicted and described in the Exemplification section. Exemplary such combinations are generally described below.

### Exemplary Ring A/Q-R¹⁰ Combinations

In some embodiments, the present invention provides a compound wherein Ring A is a 5 membered saturated or partially unsaturated carbocycle, wherein Q is a valence bond, and wherein R¹⁰ of the Q-R¹⁰ moiety is selected from the group consisting of hydrogen, halogen, an optionally substituted C₁₋₁₀ aliphatic, a suitably protected hydroxyl group, a suitably protected thiol group, a suitably protected amino group, an optionally substituted 3-8 membered saturated, partially unsaturated, or aryl monocyclic ring having 0-4 heteroatoms independently selected from nitrogen, oxygen, or sulfur, an optionally substituted 8-10 membered saturated, partially unsaturated, or aryl bicyclic ring having 0-4 heteroatoms independently selected from nitrogen, oxygen, or sulfur, a detectable moiety, a polymer residue, a peptide, a sugar-containing or sugar-like moiety, or:

wherein when R¹⁰ is a ring, R¹⁰ is optionally substituted at any substitutable carbon with 1-5 R¹¹ and at any substitutable nitrogen with R¹²;

each R¹¹ is independently halogen, R, OR, SR, N(R)₂, N(R)C(O)R, N(R)C(O)OR, N(R)C(O)N(R)₂, N(R)SO₂R, N(R)SO₂OR, S(O)R, SO₂R, OSO₂R, C(O)R, CO₂R, OCO₂R, C(O)N(R)₂, or OC(O)N(R)₂, or wherein:

two R¹¹ are optionally taken together to form an oxo moiety or an optionally substituted 3-8 membered saturated or partially unsaturated fused or spirofused ring having 0-4 heteroatoms independently selected from nitrogen, oxygen, or sulfur; and

each R¹² is independently R, OR, S(O)R, SO₂R, OSO₂R, C(O)R, CO₂R, OCO₂R, C(O)N(R)₂, or OC(O)N(R)₂, or wherein:

R¹² and R¹¹ are optionally taken together to form an optionally substituted 3-8 membered saturated or partially unsaturated fused ring having 0-4 heteroatoms independently selected from nitrogen, oxygen, or sulfur.

In certain embodiments, the present invention provides a compound wherein Ring A is a 5 membered saturated carbocycle, wherein Q is a valence bond, and wherein R¹⁰ of the Q-R¹⁰ moiety is selected from the group consisting of hydrogen, halogen, a suitably protected hydroxyl group, a suitably protected thiol group, or a suitably protected amino group.

In certain embodiments, the present invention provides a compound wherein Ring A is a 5 membered saturated carbocycle, wherein Q is a valence bond, and wherein R¹⁰ of the Q-R¹⁰ moiety is a ring optionally substituted with 1-5 R¹¹, wherein each R¹¹ is independently halogen, R, OR, SR, N(R)₂, N(R)C(O)R, N(R)C(O)OR, N(R)C(O)N(R)₂, N(R)SO₂R, N(R)SO₂OR, S(O)R, SO₂R, OSO₂R, C(O)R, CO₂R, OCO₂R, C(O)N(R)₂, or OC(O)N(R)₂, or wherein:

two R¹¹ are optionally taken together to form an oxo moiety or an optionally substituted 3-8 membered saturated or partially unsaturated fused or spirofused ring having 0-4 heteroatoms independently selected from nitrogen, oxygen, or sulfur; and

each R¹² is independently R, OR, S(O)R, SO₂R, OSO₂R, C(O)R, CO₂R, OCO₂R, C(O)N(R)₂, or OC(O)N(R)₂, or wherein:

R¹² and R¹¹ are optionally taken together to form an optionally substituted 3-8 membered saturated or partially unsaturated fused ring having 0-4 heteroatoms independently selected from nitrogen, oxygen, or sulfur.

In certain embodiments, the present invention provides a compound wherein Ring A is a 5 membered saturated carbocycle, wherein Q is a valence bond, and wherein R¹⁰ of the Q-R¹⁰ moiety is a sugar-containing or sugar-like moiety.

In certain embodiments, the present invention provides a compound wherein Ring A is a 5 membered partially unsaturated carbocycle, wherein Q is a valence bond, and wherein R¹⁰ of the Q-R¹⁰ moiety is selected from the group consisting of hydrogen, halogen, a suitably protected hydroxyl group, a suitably protected thiol group, or a suitably protected amino group.

In certain embodiments, the present invention provides a compound wherein Ring A is a 5 membered partially unsaturated carbocycle, wherein Q is a valence bond, and wherein R¹⁰ of the Q-R¹⁰ moiety is a ring optionally substituted at any substitutable carbon with 1-5 R¹¹ and at any substitutable nitrogen with R¹², wherein each R¹¹ is independently selected from halogen, R, OR, SR, N(R)₂, N(R)C(O)R, N(R)C(O)OR, N(R)C(O)N(R)₂, N(R)SO₂R, N(R)SO₂OR, S(O)R, SO₂R, OSO₂R, C(O)R, CO₂R, OCO₂R, C(O)N(R)₂, or OC(O)N(R)₂.

In certain embodiments, the present invention provides a compound wherein Ring A is a 5 membered partially unsaturated carbocycle, wherein Q is a valence bond, and wherein R¹⁰ of the Q-R¹⁰ moiety is a sugar-containing or sugar-like moiety.

In some embodiments, the present invention provides a compound wherein Ring A is a 5 membered saturated or partially unsaturated ring having 1-2 heteroatoms independently selected from nitrogen, oxygen, or sulfur, and wherein Q is a valence bond and wherein R¹⁰ of the Q-R¹⁰ moiety is selected from the group consisting of hydrogen, halogen, an optionally substituted C₁₋₁₀ aliphatic, a suitably protected hydroxyl group, a suitably protected thiol group, a suitably protected amino group, an optionally substituted 3-8 membered saturated, partially unsaturated, or aryl monocyclic ring having 0-4 heteroatoms independently selected from nitrogen, oxygen, or sulfur, an optionally substituted 8-10 membered saturated, partially unsaturated, or aryl bicyclic ring having 0-4 heteroatoms independently selected from nitrogen, oxygen, or sulfur, a detectable moiety, a polymer residue, a peptide, a sugar-containing or sugar-like moiety, or:

wherein when R¹⁰ is a ring, R¹⁰ is optionally substituted at any substitutable carbon with 1-5 R¹¹ and at any substitutable nitrogen with R¹²;

each R¹¹ is independently halogen, R, OR, SR, N(R)₂, N(R)C(O)R, N(R)C(O)OR, N(R)C(O)N(R)₂, N(R)SO₂R, N(R)SO₂OR, S(O)R, SO₂R, OSO₂R, C(O)R, CO₂R, OCO₂R, C(O)N(R)₂, or OC(O)N(R)₂, or wherein:

two R¹¹ are optionally taken together to form an oxo moiety or an optionally substituted 3-8 membered saturated or partially unsaturated fused or spirofused ring having 0-4 heteroatoms independently selected from nitrogen, oxygen, or sulfur; and

each R¹² is independently R, OR, S(O)R, SO₂R, OSO₂R, C(O)R, CO₂R, OCO₂R, C(O)N(R)₂, or OC(O)N(R)₂, or wherein:

R¹² and R¹¹ are optionally taken together to form an optionally substituted 3-8 membered saturated or partially unsaturated fused ring having 0-4 heteroatoms independently selected from nitrogen, oxygen, or sulfur.

In certain embodiments, the present invention provides a compound wherein Ring A is a 5 membered saturated ring having 1-2 heteroatoms independently selected from nitrogen, oxygen, or sulfur, wherein Q is a valence bond, and wherein R¹⁰ of the Q-R¹⁰ moiety is selected from the group consisting of hydrogen, halogen, a suitably protected hydroxyl group, a suitably protected thiol group, or a suitably protected amino group.

In certain embodiments, the present invention provides a compound wherein Ring A is a 5 membered saturated ring having 1-2 heteroatoms independently selected from nitrogen, oxygen, or sulfur, wherein Q is a valence bond, and wherein R¹⁰ of the Q-R¹⁰ moiety is a ring optionally substituted at any substitutable carbon with 1-5 R¹¹ and at any substitutable nitrogen with R¹², wherein each R¹¹ is independently selected from halogen, R, OR, SR, N(R)₂, N(R)C(O)R, N(R)C(O)OR, N(R)C(O)N(R)₂, N(R)SO₂R, N(R)SO₂OR, S(O)R, SO₂R, OSO₂R, C(O)R, CO₂R, OCO₂R, C(O)N(R)₂, or OC(O)N(R)₂.

In certain embodiments, the present invention provides a compound wherein Ring A is a 5 membered saturated ring having 1-2 heteroatoms independently selected from nitrogen, oxygen, or sulfur, wherein Q is a valence bond, and wherein R¹⁰ of the Q-R¹⁰ moiety is a sugar-containing or sugar-like moiety.

In certain embodiments, the present invention provides a compound wherein Ring A is a 5 membered partially unsaturated ring having 1-2 heteroatoms independently selected from nitrogen, oxygen, or sulfur, wherein Q is a valence bond, and wherein R¹⁰ of the Q-R¹⁰ moiety is selected from the group consisting of hydrogen, halogen, a suitably protected hydroxyl group, a suitably protected thiol group, or a suitably protected amino group.

In certain embodiments, the present invention provides a compound wherein Ring A is a 5 membered partially unsaturated ring having 1-2 heteroatoms independently selected from nitrogen, oxygen, or sulfur, wherein Q is a valence bond, and wherein R¹⁰ of the Q-R¹⁰ moiety is a ring optionally substituted at any substitutable carbon with 1-5 R¹¹ and at any substitutable nitrogen with R¹², wherein each R¹¹ is independently selected from halogen, R, OR, SR, N(R)₂, N(R)C(O)R, N(R)C(O)OR, N(R)C(O)N(R)₂, N(R)SO₂R, N(R)SO₂OR, S(O)R, SO₂R, OSO₂R, C(O)R, CO₂R, OCO₂R, C(O)N(R)₂, or OC(O)N(R)₂.

In certain embodiments, the present invention provides a compound wherein Ring A is a 5 membered partially unsaturated ring having 1-2 heteroatoms independently selected from nitrogen, oxygen, or sulfur, wherein Q is a valence bond, and wherein R¹⁰ of the Q-R¹⁰ moiety is a sugar-containing or sugar-like moiety.

In some embodiments, the present invention provides a compound wherein Ring A is a 6 membered saturated or partially unsaturated carbocycle, wherein Q is a valence bond, and wherein R¹⁰ of the Q-R¹⁰ moiety is selected from the group consisting of hydrogen, halogen, an optionally substituted C₁₋₁₀ aliphatic, a suitably protected hydroxyl group, a suitably protected thiol group, a suitably protected amino group, an optionally substituted 3-8 membered saturated, partially unsaturated, or aryl monocyclic ring having 0-4 heteroatoms independently selected from nitrogen, oxygen, or sulfur, an optionally substituted 8-10 membered saturated, partially unsaturated, or aryl bicyclic ring having 0-4 heteroatoms independently selected from nitrogen, oxygen, or sulfur, a detectable moiety, a polymer residue, a peptide, a sugar-containing or sugar-like moiety, or:

wherein when R¹⁰ is a ring, R¹⁰ is optionally substituted at any substitutable carbon with 1-5 R¹¹ and at any substitutable nitrogen with R¹²;

each R¹¹ is independently halogen, R, OR, SR, N(R)₂, N(R)C(O)R, N(R)C(O)OR, N(R)C(O)N(R)₂, N(R)SO₂R, N(R)SO₂OR, S(O)R, SO₂R, OSO₂R, C(O)R, CO₂R, OCO₂R, C(O)N(R)₂, or OC(O)N(R)₂, or wherein:

two R¹¹ are optionally taken together to form an oxo moiety or an optionally substituted 3-8 membered saturated or partially unsaturated fused or spirofused ring having 0-4 heteroatoms independently selected from nitrogen, oxygen, or sulfur; and

each R¹² is independently R, OR, S(O)R, SO₂R, OSO₂R, C(O)R, CO₂R, OCO₂R, C(O)N(R)₂, or OC(O)N(R)₂, or wherein:

R¹² and R¹¹ are optionally taken together to form an optionally substituted 3-8 membered saturated or partially unsaturated fused ring having 0-4 heteroatoms independently selected from nitrogen, oxygen, or sulfur.

In certain embodiments, the present invention provides a compound wherein Ring A is a 6 membered saturated carbocycle, wherein Q is a valence bond, and wherein R¹⁰ of the Q-R¹⁰ moiety is selected from the group consisting of hydrogen, halogen, a suitably protected hydroxyl group, a suitably protected thiol group, or a suitably protected amino group.

In certain embodiments, the present invention provides a compound wherein Ring A is a 6 membered saturated carbocycle, wherein Q is a valence bond, and wherein R¹⁰ of the Q-R¹⁰ moiety is a ring optionally substituted at any substitutable carbon with 1-5 R¹¹ and at any substitutable nitrogen with R¹², wherein each R¹¹ is independently selected from halogen, R, OR, SR, N(R)₂, N(R)C(O)R, N(R)C(O)OR, N(R)C(O)N(R)₂, N(R)SO₂R, N(R)SO₂OR, S(O)R, SO₂R, OSO₂R, C(O)R, CO₂R, OCO₂R, C(O)N(R)₂, or OC(O)N(R)₂.

In certain embodiments, the present invention provides a compound wherein Ring A is a 6 membered saturated carbocycle, wherein Q is a valence bond, and wherein R¹⁰ of the Q-R¹⁰ moiety is a sugar-containing or sugar-like moiety.

In certain embodiments, the present invention provides a compound wherein Ring A is a 6 membered partially unsaturated carbocycle, wherein Q is a valence bond, and wherein R¹⁰ of the Q-R¹⁰ moiety is selected from the group consisting of hydrogen, halogen, a suitably protected hydroxyl group, a suitably protected thiol group, or a suitably protected amino group.

In certain embodiments, the present invention provides a compound wherein Ring A is a 6 membered partially unsaturated carbocycle, wherein Q is a valence bond, and wherein R¹⁰ of the Q-R¹⁰ moiety is a ring optionally substituted at any substitutable carbon with 1-5 R¹¹ and at any substitutable nitrogen with R¹², wherein each R¹¹ is independently selected from halogen, R, OR, SR, N(R)₂, N(R)C(O)R, N(R)C(O)OR, N(R)C(O)N(R)₂, N(R)SO₂R, N(R)SO₂OR, S(O)R, SO₂R, OSO₂R, C(O)R, CO₂R, OCO₂R, C(O)N(R)₂, or OC(O)N(R)₂.

In certain embodiments, the present invention provides a compound wherein Ring A is a 6 membered partially unsaturated carbocycle, wherein Q is a valence bond, and wherein R¹⁰ of the Q-R¹⁰ moiety is a sugar-containing or sugar-like moiety.

In some embodiments, the present invention provides a compound wherein Ring A is a 6 membered saturated or partially unsaturated ring having 1-2 heteroatoms independently selected from nitrogen, oxygen, or sulfur, and wherein Q is a valence bond and wherein R¹⁰ of the Q-R¹⁰ moiety is selected from the group consisting of hydrogen, halogen, an optionally substituted C₁₋₁₀ aliphatic, a suitably protected hydroxyl group, a suitably protected thiol group, a suitably protected amino group, an optionally substituted 3-8 membered saturated, partially unsaturated, or aryl monocyclic ring having 0-4 heteroatoms independently selected from nitrogen, oxygen, or sulfur, an optionally substituted 8-10 membered saturated, partially unsaturated, or aryl bicyclic ring having 0-4 heteroatoms independently selected from nitrogen, oxygen, or sulfur, a detectable moiety, a polymer residue, a peptide, a sugar-containing or sugar-like moiety, or:

wherein when R¹⁰ is a ring, R¹⁰ is optionally substituted at any substitutable carbon with 1-5 R¹¹ and at any substitutable nitrogen with R¹²;

each R¹¹ is independently halogen, R, OR, SR, N(R)₂, N(R)C(O)R, N(R)C(O)OR, N(R)C(O)N(R)₂, N(R)SO₂R, N(R)SO₂OR, S(O)R, SO₂R, OSO₂R, C(O)R, CO₂R, OCO₂R, C(O)N(R)₂, or OC(O)N(R)₂, or wherein:

two R¹¹ are optionally taken together to form an oxo moiety or an optionally substituted 3-8 membered saturated or partially unsaturated fused or spirofused ring having 0-4 heteroatoms independently selected from nitrogen, oxygen, or sulfur; and

each R¹² is independently R, OR, S(O)R, SO₂R, OSO₂R, C(O)R, CO₂R, OCO₂R, C(O)N(R)₂, or OC(O)N(R)₂, or wherein:

R¹² and R¹¹ are optionally taken together to form an optionally substituted 3-8 membered saturated or partially unsaturated fused ring having 0-4 heteroatoms independently selected from nitrogen, oxygen, or sulfur.

In certain embodiments, the present invention provides a compound wherein Ring A is a 6 membered saturated ring having 1-2 heteroatoms independently selected from nitrogen, oxygen, or sulfur, wherein Q is a valence bond, and wherein R¹⁰ of the Q-R¹⁰ moiety is selected from the group consisting of hydrogen, halogen, a suitably protected hydroxyl group, a suitably protected thiol group, or a suitably protected amino group.

In certain embodiments, the present invention provides a compound wherein Ring A is a 6 membered saturated ring having 1-2 heteroatoms independently selected from nitrogen, oxygen, or sulfur, wherein Q is a valence bond, and wherein R¹⁰ of the Q-R¹⁰ moiety is a ring optionally substituted at any substitutable carbon with 1-5 R¹¹ and at any substitutable nitrogen with R¹², wherein each R¹¹ is independently selected from halogen, R, OR, SR, N(R)₂, N(R)C(O)R, N(R)C(O)OR, N(R)C(O)N(R)₂, N(R)SO₂R, N(R)SO₂OR, S(O)R, SO₂R, OSO₂R, C(O)R, CO₂R, OCO₂R, C(O)N(R)₂, or OC(O)N(R)₂.

In certain embodiments, the present invention provides a compound wherein Ring A is a 6 membered saturated ring having 1-2 heteroatoms independently selected from nitrogen, oxygen, or sulfur, wherein Q is a valence bond, and wherein R¹⁰ of the Q-R¹⁰ moiety is a sugar-containing or sugar-like moiety.

In certain embodiments, the present invention provides a compound wherein Ring A is a 6 membered partially unsaturated ring having 1-2 heteroatoms independently selected from nitrogen, oxygen, or sulfur, wherein Q is a valence bond, and wherein R¹⁰ of the Q-R¹⁰ moiety is selected from the group consisting of hydrogen, halogen, a suitably protected hydroxyl group, a suitably protected thiol group, or a suitably protected amino group.

In certain embodiments, the present invention provides a compound wherein Ring A is a 6 membered partially unsaturated ring having 1-2 heteroatoms independently selected from nitrogen, oxygen, or sulfur, wherein Q is a valence bond, and wherein R¹⁰ of the Q-R¹⁰ moiety is a ring optionally substituted at any substitutable carbon with 1-5 R¹¹ and at any substitutable nitrogen with R¹², wherein each R¹¹ is independently selected from halogen, R, OR, SR, N(R)₂, N(R)C(O)R, N(R)C(O)OR, N(R)C(O)N(R)₂, N(R)SO₂R, N(R)SO₂OR, S(O)R, SO₂R, OSO₂R, C(O)R, CO₂R, OCO₂R, C(O)N(R)₂, or OC(O)N(R)₂.

In certain embodiments, the present invention provides a compound wherein Ring A is a 6 membered partially unsaturated ring having 1-2 heteroatoms independently selected from nitrogen, oxygen, or sulfur, wherein Q is a valence bond, and wherein R¹⁰ of the Q-R¹⁰ moiety is a sugar-containing or sugar-like moiety.

In some embodiments, the present invention provides a compound wherein Ring A is a 7 membered saturated or partially unsaturated carbocycle, wherein Q is a valence bond, and wherein R¹⁰ of the Q-R¹⁰ moiety is selected from the group consisting of hydrogen, halogen, an optionally substituted C₁₋₁₀ aliphatic, a suitably protected hydroxyl group, a suitably protected thiol group, a suitably protected amino group, an optionally substituted 3-8 membered saturated, partially unsaturated, or aryl monocyclic ring having 0-4 heteroatoms independently selected from nitrogen, oxygen, or sulfur, an optionally substituted 8-10 membered saturated, partially unsaturated, or aryl bicyclic ring having 0-4 heteroatoms independently selected from nitrogen, oxygen, or sulfur, a detectable moiety, a polymer residue, a peptide, a sugar-containing or sugar-like moiety, or:

wherein when R¹⁰ is a ring, R¹⁰ is optionally substituted at any substitutable carbon with 1-5 R¹¹ and at any substitutable nitrogen with R¹²;

each R¹¹ is independently halogen, R, OR, SR, N(R)₂, N(R)C(O)R, N(R)C(O)OR, N(R)C(O)N(R)₂, N(R)SO₂R, N(R)SO₂OR, S(O)R, SO₂R, OSO₂R, C(O)R, CO₂R, OCO₂R, C(O)N(R)₂, or OC(O)N(R)₂, or wherein:

two R¹¹ are optionally taken together to form an oxo moiety or an optionally substituted 3-8 membered saturated or partially unsaturated fused or spirofused ring having 0-4 heteroatoms independently selected from nitrogen, oxygen, or sulfur; and

each R¹² is independently R, OR, S(O)R, SO₂R, OSO₂R, C(O)R, CO₂R, OCO₂R, C(O)N(R)₂, or OC(O)N(R)₂, or wherein:

R¹² and R¹¹ are optionally taken together to form an optionally substituted 3-8 membered saturated or partially unsaturated fused ring having 0-4 heteroatoms independently selected from nitrogen, oxygen, or sulfur.

In certain embodiments, the present invention provides a compound wherein Ring A is a 7 membered saturated carbocycle, wherein Q is a valence bond, and wherein R¹⁰ of the Q-R¹⁰ moiety is selected from the group consisting of hydrogen, halogen, a suitably protected hydroxyl group, a suitably protected thiol group, or a suitably protected amino group.

In certain embodiments, the present invention provides a compound wherein Ring A is a 7 membered saturated carbocycle, wherein Q is a valence bond, and wherein R¹⁰ of the Q-R¹⁰ moiety is a ring optionally substituted at any substitutable carbon with 1-5 R¹¹ and at any substitutable nitrogen with R¹², wherein each R¹¹ is independently selected from halogen, R, OR, SR, N(R)₂, N(R)C(O)R, N(R)C(O)OR, N(R)C(O)N(R)₂, N(R)SO₂R, N(R)SO₂OR, S(O)R, SO₂R, OSO₂R, C(O)R, CO₂R, OCO₂R, C(O)N(R)₂, or OC(O)N(R)₂.

In certain embodiments, the present invention provides a compound wherein Ring A is a 7 membered saturated carbocycle, wherein Q is a valence bond, and wherein R¹⁰ of the Q-R¹⁰ moiety is a sugar-containing or sugar-like moiety.

In certain embodiments, the present invention provides a compound wherein Ring A is a 7 membered partially unsaturated carbocycle, wherein Q is a valence bond, and wherein R¹⁰ of the Q-R¹⁰ moiety is selected from the group consisting of hydrogen, halogen, a suitably protected hydroxyl group, a suitably protected thiol group, or a suitably protected amino group.

In certain embodiments, the present invention provides a compound wherein Ring A is a 7 membered partially unsaturated carbocycle, wherein Q is a valence bond, and wherein R¹⁰ of the Q-R¹⁰ moiety is a ring optionally substituted at any substitutable carbon with 1-5 R¹¹ and at any substitutable nitrogen with R¹², wherein each R¹¹ is independently selected from halogen, R, OR, SR, N(R)₂, N(R)C(O)R, N(R)C(O)OR, N(R)C(O)N(R)₂, N(R)SO₂R, N(R)SO₂OR, S(O)R, SO₂R, OSO₂R, C(O)R, CO₂R, OCO₂R, C(O)N(R)₂, or OC(O)N(R)₂.

In certain embodiments, the present invention provides a compound wherein Ring A is a 7 membered partially unsaturated carbocycle, wherein Q is a valence bond, and wherein R¹⁰ of the Q-R¹⁰ moiety is a sugar-containing or sugar-like moiety.

In some embodiments, the present invention provides a compound wherein Ring A is a 7 membered saturated or partially unsaturated ring having 1-2 heteroatoms independently selected from nitrogen, oxygen, or sulfur, and wherein Q is a valence bond and wherein R¹⁰ of the Q-R¹⁰ moiety is selected from the group consisting of hydrogen, halogen, an optionally substituted C₁₋₁₀ aliphatic, a suitably protected hydroxyl group, a suitably protected thiol group, a suitably protected amino group, an optionally substituted 3-8 membered saturated, partially unsaturated, or aryl monocyclic ring having 0-4 heteroatoms independently selected from nitrogen, oxygen, or sulfur, an optionally substituted 8-10 membered saturated, partially unsaturated, or aryl bicyclic ring having 0-4 heteroatoms independently selected from nitrogen, oxygen, or sulfur, a detectable moiety, a polymer residue, a peptide, a sugar-containing or sugar-like moiety, or:

wherein when R¹⁰ is a ring, R¹⁰ is optionally substituted at any substitutable carbon with 1-5 R¹¹ and at any substitutable nitrogen with R¹²;

each R¹¹ is independently halogen, R, OR, SR, N(R)₂, N(R)C(O)R, N(R)C(O)OR, N(R)C(O)N(R)₂, N(R)SO₂R, N(R)SO₂OR, S(O)R, SO₂R, OSO₂R, C(O)R, CO₂R, OCO₂R, C(O)N(R)₂, or OC(O)N(R)₂, or wherein:

two R¹¹ are optionally taken together to form an oxo moiety or an optionally substituted 3-8 membered saturated or partially unsaturated fused or spirofused ring having 0-4 heteroatoms independently selected from nitrogen, oxygen, or sulfur; and

each R¹² is independently R, OR, S(O)R, SO₂R, OSO₂R, C(O)R, CO₂R, OCO₂R, C(O)N(R)₂, or OC(O)N(R)₂, or wherein:

R¹² and R¹¹ are optionally taken together to form an optionally substituted 3-8 membered saturated or partially unsaturated fused ring having 0-4 heteroatoms independently selected from nitrogen, oxygen, or sulfur.

In certain embodiments, the present invention provides a compound wherein Ring A is a 7 membered saturated ring having 1-2 heteroatoms independently selected from nitrogen, oxygen, or sulfur, wherein Q is a valence bond, and wherein R¹⁰ of the Q-R¹⁰ moiety is selected from the group consisting of hydrogen, halogen, a suitably protected hydroxyl group, a suitably protected thiol group, or a suitably protected amino group.

In certain embodiments, the present invention provides a compound wherein Ring A is a 7 membered saturated ring having 1-2 heteroatoms independently selected from nitrogen, oxygen, or sulfur, wherein Q is a valence bond, and wherein R¹⁰ of the Q-R¹⁰ moiety is a ring optionally substituted at any substitutable carbon with 1-5 R¹¹ and at any substitutable nitrogen with R¹², wherein each R¹¹ is independently selected from halogen, R, OR, SR, N(R)₂, N(R)C(O)R, N(R)C(O)OR, N(R)C(O)N(R)₂, N(R)SO₂R, N(R)SO₂OR, S(O)R, SO₂R, OSO₂R, C(O)R, CO₂R, OCO₂R, C(O)N(R)₂, or OC(O)N(R)₂.

In certain embodiments, the present invention provides a compound wherein Ring A is a 7 membered saturated ring having 1-2 heteroatoms independently selected from nitrogen, oxygen, or sulfur, wherein Q is a valence bond, and wherein R¹⁰ of the Q-R¹⁰ moiety is a sugar-containing or sugar-like moiety.

In certain embodiments, the present invention provides a compound wherein Ring A is a 7 membered partially unsaturated ring having 1-2 heteroatoms independently selected from nitrogen, oxygen, or sulfur, wherein Q is a valence bond, and wherein R¹⁰ of the Q-R¹⁰ moiety is selected from the group consisting of hydrogen, halogen, a suitably protected hydroxyl group, a suitably protected thiol group, or a suitably protected amino group.

In certain embodiments, the present invention provides a compound wherein Ring A is a 7 membered partially unsaturated ring having 1-2 heteroatoms independently selected from nitrogen, oxygen, or sulfur, wherein Q is a valence bond, and wherein R¹⁰ of the Q-R¹⁰ moiety is a ring optionally substituted at any substitutable carbon with 1-5 R¹¹ and at any substitutable nitrogen with R¹², wherein each R¹¹ is independently selected from halogen, R, OR, SR, N(R)₂, N(R)C(O)R, N(R)C(O)OR, N(R)C(O)N(R)₂, N(R)SO₂R, N(R)SO₂OR, S(O)R, SO₂R, OSO₂R, C(O)R, CO₂R, OCO₂R, C(O)N(R)₂, or OC(O)N(R)₂.

In certain embodiments, the present invention provides a compound wherein Ring A is a 7 membered partially unsaturated ring having 1-2 heteroatoms independently selected from nitrogen, oxygen, or sulfur, wherein Q is a valence bond, and wherein R¹⁰ of the Q-R¹⁰ moiety is a sugar-containing or sugar-like moiety.

In some embodiments, the present invention provides a compound wherein Ring A is a 5 membered saturated or partially unsaturated carbocycle, wherein Q is an optionally substituted C₁₋₁₀ alkylene chain wherein one, two, or three methylene units of Q are optionally and independently replaced by —O—, —N(R)—, —S—, —C(O)—, —OC(O)—, —C(O)O—, —OC(O)O—, —S(O)—, or —S(O)₂—, —OSO₂O—, —N(R)C(O)—, —C(O)NR—, —N(R)C(O)O—, —OC(O)NR—, —N(R)C(O)NR—, or —Cy-, wherein:

each —Cy- is independently a bivalent optionally substituted saturated, partially unsaturated, or aromatic monocyclic or bicyclic ring selected from a 6-10 membered arylene, a 5-10 membered heteroarylene having 1-4 heteroatoms independently selected from oxygen, nitrogen, or sulfur, a 3-8 membered carbocyclylene, or a 3-10 membered heterocyclylene having 1-4 heteroatoms independently selected from oxygen, nitrogen, or sulfur, and:

wherein R¹⁰ of the Q-R¹⁰ moiety is selected from the group consisting of hydrogen, halogen, an optionally substituted C₁₋₁₀ aliphatic, a suitably protected hydroxyl group, a suitably protected thiol group, a suitably protected amino group, an optionally substituted 3-8 membered saturated, partially unsaturated, or aryl monocyclic ring having 0-4 heteroatoms independently selected from nitrogen, oxygen, or sulfur, an optionally substituted 8-10 membered saturated, partially unsaturated, or aryl bicyclic ring having 0-4 heteroatoms independently selected from nitrogen, oxygen, or sulfur, a detectable moiety, a polymer residue, a peptide, a sugar-containing or sugar-like moiety, or:

wherein when R¹⁰ is a ring, R¹⁰ is optionally substituted at any substitutable carbon with 1-5 R¹¹ and at any substitutable nitrogen with R¹²;

each R¹¹ is independently halogen, R, OR, SR, N(R)₂, N(R)C(O)R, N(R)C(O)OR, N(R)C(O)N(R)₂, N(R)SO₂R, N(R)SO₂OR, S(O)R, SO₂R, OSO₂R, C(O)R, CO₂R, OCO₂R, C(O)N(R)₂, or OC(O)N(R)₂, or wherein:

two R¹¹ are optionally taken together to form an oxo moiety or an optionally substituted 3-8 membered saturated or partially unsaturated fused or spirofused ring having 0-4 heteroatoms independently selected from nitrogen, oxygen, or sulfur; and

each R¹² is independently R, OR, S(O)R, SO₂R, OSO₂R, C(O)R, CO₂R, OCO₂R, C(O)N(R)₂, or OC(O)N(R)₂, or wherein:

R¹² and R¹¹ are optionally taken together to form an optionally substituted 3-8 membered saturated or partially unsaturated fused ring having 0-4 heteroatoms independently selected from nitrogen, oxygen, or sulfur.

In certain embodiments, the present invention provides a compound wherein Ring A is a 5 membered saturated carbocycle, wherein Q is an optionally substituted C₁₋₁₀ alkylene chain wherein one, two, or three methylene units of Q are optionally and independently replaced by —O—, —N(R)—, —S—, or —Cy-, and wherein R¹⁰ of the Q-R¹⁰ moiety is selected from the group consisting of hydrogen, halogen, a suitably protected hydroxyl group, a suitably protected thiol group, or a suitably protected amino group.

In certain embodiments, the present invention provides a compound wherein Ring A is a 5 membered saturated carbocycle, wherein Q is an optionally substituted C₁₋₁₀ alkylene chain wherein one, two, or three methylene units of Q are optionally and independently replaced by —O—, —N(R)—, —S—, or —Cy-, and wherein R¹⁰ of the Q-R¹⁰ moiety is a ring optionally substituted at any substitutable carbon with 1-5 R¹¹ and at any substitutable nitrogen with R¹², wherein each R¹¹ is independently selected from halogen, R, OR, SR, N(R)₂, N(R)C(O)R, N(R)C(O)OR, N(R)C(O)N(R)₂, N(R)SO₂R, N(R)SO₂OR, S(O)R, SO₂R, OSO₂R, C(O)R, CO₂R, OCO₂R, C(O)N(R)₂, or OC(O)N(R)₂. In certain embodiments, the compound is as described above and R¹⁰ is an optionally substituted heterocycle. In certain embodiments, the compound is as described above and R¹⁰ is an optionally substituted 5-6 membered heterocycle with 1-3 heteroatoms. In certain embodiments, the compound is as described above and R¹⁰ is an optionally substituted 6 membered heterocycle with 2 heteroatoms. In certain embodiments, the compound is as described above and R¹⁰ is optionally substituted morpholine.

In certain embodiments, the present invention provides a compound wherein Ring A is a 5 membered saturated carbocycle, wherein Q is an optionally substituted C₁₋₁₀ alkylene chain wherein one, two, or three methylene units of Q are optionally and independently replaced by —O—, —N(R)—, —S—, or —Cy-, and wherein R¹⁰ of the Q-R¹⁰ moiety is a sugar-containing or sugar-like moiety.

In certain embodiments, the present invention provides a compound wherein Ring A is a 5 membered partially unsaturated carbocycle, wherein Q is an optionally substituted C₁₋₁₀ alkylene chain wherein one, two, or three methylene units of Q are optionally and independently replaced by —O—, —N(R)—, —S—, or —Cy-, and wherein R¹⁰ of the Q-R¹⁰ moiety is selected from the group consisting of hydrogen, halogen, a suitably protected hydroxyl group, a suitably protected thiol group, or a suitably protected amino group.

In certain embodiments, the present invention provides a compound wherein Ring A is a 5 membered partially unsaturated carbocycle, wherein Q is an optionally substituted C₁₋₁₀ alkylene chain wherein one, two, or three methylene units of Q are optionally and independently replaced by —O—, —N(R)—, —S—, or —Cy-, and wherein R¹⁰ of the Q-R¹⁰ moiety is a ring optionally substituted at any substitutable carbon with 1-5 R¹¹ and at any substitutable nitrogen with R¹², wherein each R¹¹ is independently selected from halogen, R, OR, SR, N(R)₂, N(R)C(O)R, N(R)C(O)OR, N(R)C(O)N(R)₂, N(R)SO₂R, N(R)SO₂OR, S(O)R, SO₂R, OSO₂R, C(O)R, CO₂R, OCO₂R, C(O)N(R)₂, or OC(O)N(R)₂. In certain embodiments, the compound is as described above and R¹⁰ is an optionally substituted heterocycle. In certain embodiments, the compound is as described above and R¹⁰ is an optionally substituted 5-6 membered heterocycle with 1-3 heteroatoms. In certain embodiments, the compound is as described above and R¹⁰ is an optionally substituted 6 membered heterocycle with 2 heteroatoms. In certain embodiments, the compound is as described above and R¹⁰ is optionally substituted morpholine.

In certain embodiments, the present invention provides a compound wherein Ring A is a 5 membered partially unsaturated carbocycle, wherein Q is an optionally substituted C₁₋₁₀ alkylene chain wherein one, two, or three methylene units of Q are optionally and independently replaced by —O—, —N(R)—, —S—, or —Cy-, and wherein R¹⁰ of the Q-R¹⁰ moiety is a sugar-containing or sugar-like moiety.

In some embodiments, the present invention provides a compound wherein Ring A is a 5 membered saturated or partially unsaturated ring having 1-2 heteroatoms independently selected from nitrogen, oxygen, or sulfur, and wherein Q is an optionally substituted C₁₋₁₀ alkylene chain wherein one, two, or three methylene units of Q are optionally and independently replaced by —O—, —N(R)—, —S—, —C(O)—, —OC(O)—, —C(O)O—, —OC(O)O—, —S(O)—, or —S(O)₂—, —OSO₂O—, —N(R)C(O)—, —C(O)NR—, —N(R)C(O)O—, —OC(O)NR—, —N(R)C(O)NR—, or —Cy-, wherein:

each —Cy- is independently a bivalent optionally substituted saturated, partially unsaturated, or aromatic monocyclic or bicyclic ring selected from a 6-10 membered arylene, a 5-10 membered heteroarylene having 1-4 heteroatoms independently selected from oxygen, nitrogen, or sulfur, a 3-8 membered carbocyclylene, or a 3-10 membered heterocyclylene having 1-4 heteroatoms independently selected from oxygen, nitrogen, or sulfur, and:

wherein R¹⁰ of the Q-R¹⁰ moiety is selected from the group consisting of hydrogen, halogen, an optionally substituted C₁₋₁₀ aliphatic, a suitably protected hydroxyl group, a suitably protected thiol group, a suitably protected amino group, an optionally substituted 3-8 membered saturated, partially unsaturated, or aryl monocyclic ring having 0-4 heteroatoms independently selected from nitrogen, oxygen, or sulfur, an optionally substituted 8-10 membered saturated, partially unsaturated, or aryl bicyclic ring having 0-4 heteroatoms independently selected from nitrogen, oxygen, or sulfur, a detectable moiety, a polymer residue, a peptide, a sugar-containing or sugar-like moiety, or:

wherein when R¹⁰ is a ring, R¹⁰ is optionally substituted at any substitutable carbon with 1-5 R¹¹ and at any substitutable nitrogen with R¹²;

each R¹¹ is independently halogen, R, OR, SR, N(R)₂, N(R)C(O)R, N(R)C(O)OR, N(R)C(O)N(R)₂, N(R)SO₂R, N(R)SO₂OR, S(O)R, SO₂R, OSO₂R, C(O)R, CO₂R, OCO₂R, C(O)N(R)₂, or OC(O)N(R)₂, or wherein:

two R¹¹ are optionally taken together to form an oxo moiety or an optionally substituted 3-8 membered saturated or partially unsaturated fused or spirofused ring having 0-4 heteroatoms independently selected from nitrogen, oxygen, or sulfur; and

each R¹² is independently R, OR, S(O)R, SO₂R, OSO₂R, C(O)R, CO₂R, OCO₂R, C(O)N(R)₂, or OC(O)N(R)₂, or wherein:

R¹² and R¹¹ are optionally taken together to form an optionally substituted 3-8 membered saturated or partially unsaturated fused ring having 0-4 heteroatoms independently selected from nitrogen, oxygen, or sulfur.

In certain embodiments, the present invention provides a compound wherein Ring A is a 5 membered saturated ring having 1-2 heteroatoms independently selected from nitrogen, oxygen, or sulfur, wherein Q is an optionally substituted C₁₋₁₀ alkylene chain wherein one, two, or three methylene units of Q are optionally and independently replaced by —O—, —N(R)—, —S—, or —Cy-, and wherein R¹⁰ of the Q-R¹⁰ moiety is selected from the group consisting of hydrogen, halogen, a suitably protected hydroxyl group, a suitably protected thiol group, or a suitably protected amino group.

In certain embodiments, the present invention provides a compound wherein Ring A is a 5 membered saturated ring having 1-2 heteroatoms independently selected from nitrogen, oxygen, or sulfur, wherein Q is an optionally substituted C₁₋₁₀ alkylene chain wherein one, two, or three methylene units of Q are optionally and independently replaced by —O—, —N(R)—, —S—, or —Cy-, and wherein R¹⁰ of the Q-R¹⁰ moiety is a ring optionally substituted at any substitutable carbon with 1-5 R¹¹ and at any substitutable nitrogen with R¹², wherein each R¹¹ is independently selected from halogen, R, OR, SR, N(R)₂, N(R)C(O)R, N(R)C(O)OR, N(R)C(O)N(R)₂, N(R)SO₂R, N(R)SO₂OR, S(O)R, SO₂R, OSO₂R, C(O)R, CO₂R, OCO₂R, C(O)N(R)₂, or OC(O)N(R)₂. In certain embodiments, the compound is as described above and R¹⁰ is an optionally substituted heterocycle. In certain embodiments, the compound is as described above and R¹⁰ is an optionally substituted 5-6 membered heterocycle with 1-3 heteroatoms. In certain embodiments, the compound is as described above and R¹⁰ is an optionally substituted 6 membered heterocycle with 2 heteroatoms. In certain embodiments, the compound is as described above and R¹⁰ is optionally substituted morpholine.

In certain embodiments, the present invention provides a compound wherein Ring A is a 5 membered saturated ring having 1-2 heteroatoms independently selected from nitrogen, oxygen, or sulfur, wherein Q is an optionally substituted C₁₋₁₀ alkylene chain wherein one, two, or three methylene units of Q are optionally and independently replaced by —O—, —N(R)—, —S—, or —Cy-, and wherein R¹⁰ of the Q-R¹⁰ moiety is a sugar-containing or sugar-like moiety.

In certain embodiments, the present invention provides a compound wherein Ring A is a 5 membered partially unsaturated ring having 1-2 heteroatoms independently selected from nitrogen, oxygen, or sulfur, wherein Q is an optionally substituted C₁₋₁₀ alkylene chain wherein one, two, or three methylene units of Q are optionally and independently replaced by —O—, —N(R)—, —S—, or —Cy-, and wherein R¹⁰ of the Q-R¹⁰ moiety is selected from the group consisting of hydrogen, halogen, a suitably protected hydroxyl group, a suitably protected thiol group, or a suitably protected amino group.

In certain embodiments, the present invention provides a compound wherein Ring A is a 5 membered partially unsaturated ring having 1-2 heteroatoms independently selected from nitrogen, oxygen, or sulfur, wherein Q is an optionally substituted C₁₋₁₀ alkylene chain wherein one, two, or three methylene units of Q are optionally and independently replaced by —O—, —N(R)—, —S—, or —Cy-, and wherein R¹⁰ of the Q-R¹⁰ moiety is a ring optionally substituted at any substitutable carbon with 1-5 R¹¹ and at any substitutable nitrogen with R¹², wherein each R¹¹ is independently selected from halogen, R, OR, SR, N(R)₂, N(R)C(O)R, N(R)C(O)OR, N(R)C(O)N(R)₂, N(R)SO₂R, N(R)SO₂OR, S(O)R, SO₂R, OSO₂R, C(O)R, CO₂R, OCO₂R, C(O)N(R)₂, or OC(O)N(R)₂. In certain embodiments, the compound is as described above and R¹⁰ is an optionally substituted heterocycle. In certain embodiments, the compound is as described above and R¹⁰ is an optionally substituted 5-6 membered heterocycle with 1-3 heteroatoms. In certain embodiments, the compound is as described above and R¹⁰ is an optionally substituted 6 membered heterocycle with 2 heteroatoms. In certain embodiments, the compound is as described above and R¹⁰ is optionally substituted morpholine.

In certain embodiments, the present invention provides a compound wherein Ring A is a 5 membered partially unsaturated ring having 1-2 heteroatoms independently selected from nitrogen, oxygen, or sulfur, wherein Q is an optionally substituted C₁₋₁₀ alkylene chain wherein one, two, or three methylene units of Q are optionally and independently replaced by —O—, —N(R)—, —S—, or —Cy-, and wherein R¹⁰ of the Q-R¹⁰ moiety is a sugar-containing or sugar-like moiety.

In some embodiments, the present invention provides a compound wherein Ring A is a 6 membered saturated or partially unsaturated carbocycle, wherein Q is an optionally substituted C₁₋₁₀ alkylene chain wherein one, two, or three methylene units of Q are optionally and independently replaced by —O—, —N(R)—, —S—, —C(O)—, —OC(O)—, —C(O)O—, —OC(O)O—, —S(O)—, or —S(O)₂—, —OSO₂O—, —N(R)C(O)—, —C(O)NR—, —N(R)C(O)O—, —OC(O)NR—, —N(R)C(O)NR—, or —Cy-, wherein:

each —Cy- is independently a bivalent optionally substituted saturated, partially unsaturated, or aromatic monocyclic or bicyclic ring selected from a 6-10 membered arylene, a 5-10 membered heteroarylene having 1-4 heteroatoms independently selected from oxygen, nitrogen, or sulfur, a 3-8 membered carbocyclylene, or a 3-10 membered heterocyclylene having 1-4 heteroatoms independently selected from oxygen, nitrogen, or sulfur, and:

wherein R¹⁰ of the Q-R¹⁰ moiety is selected from the group consisting of hydrogen, halogen, an optionally substituted C₁₋₁₀ aliphatic, a suitably protected hydroxyl group, a suitably protected thiol group, a suitably protected amino group, an optionally substituted 3-8 membered saturated, partially unsaturated, or aryl monocyclic ring having 0-4 heteroatoms independently selected from nitrogen, oxygen, or sulfur, an optionally substituted 8-10 membered saturated, partially unsaturated, or aryl bicyclic ring having 0-4 heteroatoms independently selected from nitrogen, oxygen, or sulfur, a detectable moiety, a polymer residue, a peptide, a sugar-containing or sugar-like moiety, or:

wherein when R¹⁰ is a ring, R¹⁰ is optionally substituted at any substitutable carbon with 1-5 R¹¹ and at any substitutable nitrogen with R¹²;

each R¹¹ is independently halogen, R, OR, SR, N(R)₂, N(R)C(O)R, N(R)C(O)OR, N(R)C(O)N(R)₂, N(R)SO₂R, N(R)SO₂OR, S(O)R, SO₂R, OSO₂R, C(O)R, CO₂R, OCO₂R, C(O)N(R)₂, or OC(O)N(R)₂, or wherein:

two R¹¹ are optionally taken together to form an oxo moiety or an optionally substituted 3-8 membered saturated or partially unsaturated fused or spirofused ring having 0-4 heteroatoms independently selected from nitrogen, oxygen, or sulfur; and

each R¹² is independently R, OR, S(O)R, SO₂R, OSO₂R, C(O)R, CO₂R, OCO₂R, C(O)N(R)₂, or OC(O)N(R)₂, or wherein:

R¹² and R¹¹ are optionally taken together to form an optionally substituted 3-8 membered saturated or partially unsaturated fused ring having 0-4 heteroatoms independently selected from nitrogen, oxygen, or sulfur.

In certain embodiments, the present invention provides a compound wherein Ring A is a 6 membered saturated carbocycle, wherein Q is an optionally substituted C₁₋₁₀ alkylene chain wherein one, two, or three methylene units of Q are optionally and independently replaced by —O—, —N(R)—, —S—, or —Cy-, and wherein R¹⁰ of the Q-R¹⁰ moiety is selected from the group consisting of hydrogen, halogen, a suitably protected hydroxyl group, a suitably protected thiol group, or a suitably protected amino group.

In certain embodiments, the present invention provides a compound wherein Ring A is a 6 membered saturated carbocycle, wherein Q is an optionally substituted C₁₋₁₀ alkylene chain wherein one, two, or three methylene units of Q are optionally and independently replaced by —O—, —N(R)—, —S—, or —Cy-, and wherein R¹⁰ of the Q-R¹⁰ moiety is a ring optionally substituted at any substitutable carbon with 1-5 R¹¹ and at any substitutable nitrogen with R¹², wherein each R¹¹ is independently selected from halogen, R, OR, SR, N(R)₂, N(R)C(O)R, N(R)C(O)OR, N(R)C(O)N(R)₂, N(R)SO₂R, N(R)SO₂OR, S(O)R, SO₂R, OSO₂R, C(O)R, CO₂R, OCO₂R, C(O)N(R)₂, or OC(O)N(R)₂. In certain embodiments, the compound is as described above and R¹⁰ is an optionally substituted heterocycle. In certain embodiments, the compound is as described above and R¹⁰ is an optionally substituted 5-6 membered heterocycle with 1-3 heteroatoms. In certain embodiments, the compound is as described above and R¹⁰ is an optionally substituted 6 membered heterocycle with 2 heteroatoms. In certain embodiments, the compound is as described above and R¹⁰ is optionally substituted morpholine.

In certain embodiments, the present invention provides a compound wherein Ring A is a 6 membered saturated carbocycle, wherein Q is an optionally substituted C₁₋₁₀ alkylene chain wherein one, two, or three methylene units of Q are optionally and independently replaced by —O—, —N(R)—, —S—, or —Cy-, and wherein R¹⁰ of the Q-R¹⁰ moiety is a sugar-containing or sugar-like moiety.

In certain embodiments, the present invention provides a compound wherein Ring A is a 6 membered partially unsaturated carbocycle, wherein Q is an optionally substituted C₁₋₁₀ alkylene chain wherein one, two, or three methylene units of Q are optionally and independently replaced by —O—, —N(R)—, —S—, or —Cy-, and wherein R¹⁰ of the Q-R¹⁰ moiety is selected from the group consisting of hydrogen, halogen, a suitably protected hydroxyl group, a suitably protected thiol group, or a suitably protected amino group.

In certain embodiments, the present invention provides a compound wherein Ring A is a 6 membered partially unsaturated carbocycle, wherein Q is an optionally substituted C₁₋₁₀ alkylene chain wherein one, two, or three methylene units of Q are optionally and independently replaced by —O—, —N(R)—, —S—, or —Cy-, and wherein R¹⁰ of the Q-R¹⁰ moiety is a ring optionally substituted at any substitutable carbon with 1-5 R¹¹ and at any substitutable nitrogen with R¹², wherein each R¹¹ is independently selected from halogen, R, OR, SR, N(R)₂, N(R)C(O)R, N(R)C(O)OR, N(R)C(O)N(R)₂, N(R)SO₂R, N(R)SO₂OR, S(O)R, SO₂R, OSO₂R, C(O)R, CO₂R, OCO₂R, C(O)N(R)₂, or OC(O)N(R)₂. In certain embodiments, the compound is as described above and R¹⁰ is an optionally substituted heterocycle. In certain embodiments, the compound is as described above and R¹⁰ is an optionally substituted 5-6 membered heterocycle with 1-3 heteroatoms. In certain embodiments, the compound is as described above and R¹⁰ is an optionally substituted 6 membered heterocycle with 2 heteroatoms. In certain embodiments, the compound is as described above and R¹⁰ is optionally substituted morpholine.

In certain embodiments, the present invention provides a compound wherein Ring A is a 6 membered partially unsaturated carbocycle, wherein Q is an optionally substituted C₁₋₁₀ alkylene chain wherein one, two, or three methylene units of Q are optionally and independently replaced by —O—, —N(R)—, —S—, or —Cy-, and wherein R¹⁰ of the Q-R¹⁰ moiety is a sugar-containing or sugar-like moiety.

In some embodiments, the present invention provides a compound wherein Ring A is a 6 membered saturated or partially unsaturated ring having 1-2 heteroatoms independently selected from nitrogen, oxygen, or sulfur, and wherein Q is an optionally substituted C₁₋₁₀ alkylene chain wherein one, two, or three methylene units of Q are optionally and independently replaced by —O—, —N(R)—, —S—, —C(O)—, —OC(O)—, —C(O)O—, —OC(O)O—, —S(O)—, or —S(O)₂—, —OSO₂O—, —N(R)C(O)—, —C(O)NR—, —N(R)C(O)O—, —OC(O)NR—, —N(R)C(O)NR—, or —Cy-, wherein:

each —Cy- is independently a bivalent optionally substituted saturated, partially unsaturated, or aromatic monocyclic or bicyclic ring selected from a 6-10 membered arylene, a 5-10 membered heteroarylene having 1-4 heteroatoms independently selected from oxygen, nitrogen, or sulfur, a 3-8 membered carbocyclylene, or a 3-10 membered heterocyclylene having 1-4 heteroatoms independently selected from oxygen, nitrogen, or sulfur, and:

wherein R¹⁰ of the Q-R¹⁰ moiety is selected from the group consisting of hydrogen, halogen, an optionally substituted C₁₋₁₀ aliphatic, a suitably protected hydroxyl group, a suitably protected thiol group, a suitably protected amino group, an optionally substituted 3-8 membered saturated, partially unsaturated, or aryl monocyclic ring having 0-4 heteroatoms independently selected from nitrogen, oxygen, or sulfur, an optionally substituted 8-10 membered saturated, partially unsaturated, or aryl bicyclic ring having 0-4 heteroatoms independently selected from nitrogen, oxygen, or sulfur, a detectable moiety, a polymer residue, a peptide, a sugar-containing or sugar-like moiety, or:

wherein when R¹⁰ is a ring, R¹⁰ is optionally substituted at any substitutable carbon with 1-5 R¹¹ and at any substitutable nitrogen with R¹²;

each R¹¹ is independently halogen, R, OR, SR, N(R)₂, N(R)C(O)R, N(R)C(O)OR, N(R)C(O)N(R)₂, N(R)SO₂R, N(R)SO₂OR, S(O)R, SO₂R, OSO₂R, C(O)R, CO₂R, OCO₂R, C(O)N(R)₂, or OC(O)N(R)₂, or wherein:

two R¹¹ are optionally taken together to form an oxo moiety or an optionally substituted 3-8 membered saturated or partially unsaturated fused or spirofused ring having 0-4 heteroatoms independently selected from nitrogen, oxygen, or sulfur; and

each R¹² is independently R, OR, S(O)R, SO₂R, OSO₂R, C(O)R, CO₂R, OCO₂R, C(O)N(R)₂, or OC(O)N(R)₂, or wherein:

R¹² and R¹¹ are optionally taken together to form an optionally substituted 3-8 membered saturated or partially unsaturated fused ring having 0-4 heteroatoms independently selected from nitrogen, oxygen, or sulfur.

In certain embodiments, the present invention provides a compound wherein Ring A is a 6 membered saturated ring having 1-2 heteroatoms independently selected from nitrogen, oxygen, or sulfur, wherein Q is an optionally substituted C₁₋₁₀ alkylene chain wherein one, two, or three methylene units of Q are optionally and independently replaced by —O—, —N(R)—, —S—, or —Cy-, and wherein R¹⁰ of the Q-R¹⁰ moiety is selected from the group consisting of hydrogen, halogen, a suitably protected hydroxyl group, a suitably protected thiol group, or a suitably protected amino group.

In certain embodiments, the present invention provides a compound wherein Ring A is a 6 membered saturated ring having 1-2 heteroatoms independently selected from nitrogen, oxygen, or sulfur, wherein Q is an optionally substituted C₁₋₁₀ alkylene chain wherein one, two, or three methylene units of Q are optionally and independently replaced by —O—, —N(R)—, —S—, or —Cy-, and wherein R¹⁰ of the Q-R¹⁰ moiety is a ring optionally substituted at any substitutable carbon with 1-5 R¹¹ and at any substitutable nitrogen with R¹², wherein each R¹¹ is independently selected from halogen, R, OR, SR, N(R)₂, N(R)C(O)R, N(R)C(O)OR, N(R)C(O)N(R)₂, N(R)SO₂R, N(R)SO₂OR, S(O)R, SO₂R, OSO₂R, C(O)R, CO₂R, OCO₂R, C(O)N(R)₂, or OC(O)N(R)₂. In certain embodiments, the compound is as described above and R¹⁰ is an optionally substituted heterocycle. In certain embodiments, the compound is as described above and R¹⁰ is an optionally substituted 5-6 membered heterocycle with 1-3 heteroatoms. In certain embodiments, the compound is as described above and R¹⁰ is an optionally substituted 6 membered heterocycle with 2 heteroatoms. In certain embodiments, the compound is as described above and R¹⁰ is optionally substituted morpholine.

In certain embodiments, the present invention provides a compound wherein Ring A is a 6 membered saturated ring having 1-2 heteroatoms independently selected from nitrogen, oxygen, or sulfur, wherein Q is an optionally substituted C₁₋₁₀ alkylene chain wherein one, two, or three methylene units of Q are optionally and independently replaced by —O—, —N(R)—, —S—, or —Cy-, and wherein R¹⁰ of the Q-R¹⁰ moiety is a sugar-containing or sugar-like moiety.

In certain embodiments, the present invention provides a compound wherein Ring A is a 6 membered partially unsaturated ring having 1-2 heteroatoms independently selected from nitrogen, oxygen, or sulfur, wherein Q is an optionally substituted C₁₋₁₀ alkylene chain wherein one, two, or three methylene units of Q are optionally and independently replaced by —O—, —N(R)—, —S—, or —Cy-, and wherein R¹⁰ of the Q-R¹⁰ moiety is selected from the group consisting of hydrogen, halogen, a suitably protected hydroxyl group, a suitably protected thiol group, or a suitably protected amino group.

In certain embodiments, the present invention provides a compound wherein Ring A is a 6 membered partially unsaturated ring having 1-2 heteroatoms independently selected from nitrogen, oxygen, or sulfur, wherein Q is an optionally substituted C₁₋₁₀ alkylene chain wherein one, two, or three methylene units of Q are optionally and independently replaced by —O—, —N(R)—, —S—, or —Cy-, and wherein R¹⁰ of the Q-R¹⁰ moiety is a ring optionally substituted at any substitutable carbon with 1-5 R¹¹ and at any substitutable nitrogen with R¹², wherein each R¹¹ is independently selected from halogen, R, OR, SR, N(R)₂, N(R)C(O)R, N(R)C(O)OR, N(R)C(O)N(R)₂, N(R)SO₂R, N(R)SO₂OR, S(O)R, SO₂R, OSO₂R, C(O)R, CO₂R, OCO₂R, C(O)N(R)₂, or OC(O)N(R)₂. In certain embodiments, the compound is as described above and R¹⁰ is an optionally substituted heterocycle. In certain embodiments, the compound is as described above and R¹⁰ is an optionally substituted 5-6 membered heterocycle with 1-3 heteroatoms. In certain embodiments, the compound is as described above and R¹⁰ is an optionally substituted 6 membered heterocycle with 2 heteroatoms. In certain embodiments, the compound is as described above and R¹⁰ is optionally substituted morpholine.

In certain embodiments, the present invention provides a compound wherein Ring A is a 6 membered partially unsaturated ring having 1-2 heteroatoms independently selected from nitrogen, oxygen, or sulfur, wherein Q is an optionally substituted C₁₋₁₀ alkylene chain wherein one, two, or three methylene units of Q are optionally and independently replaced by —O—, —N(R)—, —S—, or —Cy-, and wherein R¹⁰ of the Q-R¹⁰ moiety is a sugar-containing or sugar-like moiety.

In some embodiments, the present invention provides a compound wherein Ring A is a 7 membered saturated or partially unsaturated carbocycle, wherein Q is an optionally substituted C₁₋₁₀ alkylene chain wherein one, two, or three methylene units of Q are optionally and independently replaced by —O—, —N(R)—, —S—, —C(O)—, —OC(O)—, —C(O)O—, —OC(O)O—, —S(O)—, or —S(O)₂—, —OSO₂O—, —N(R)C(O)—, —C(O)NR—, —N(R)C(O)O—, —OC(O)NR—, —N(R)C(O)NR—, or —Cy-, wherein:

each —Cy- is independently a bivalent optionally substituted saturated, partially unsaturated, or aromatic monocyclic or bicyclic ring selected from a 6-10 membered arylene, a 5-10 membered heteroarylene having 1-4 heteroatoms independently selected from oxygen, nitrogen, or sulfur, a 3-8 membered carbocyclylene, or a 3-10 membered heterocyclylene having 1-4 heteroatoms independently selected from oxygen, nitrogen, or sulfur, and:

wherein R¹⁰ of the Q-R¹⁰ moiety is selected from the group consisting of hydrogen, halogen, an optionally substituted C₁₋₁₀ aliphatic, a suitably protected hydroxyl group, a suitably protected thiol group, a suitably protected amino group, an optionally substituted 3-8 membered saturated, partially unsaturated, or aryl monocyclic ring having 0-4 heteroatoms independently selected from nitrogen, oxygen, or sulfur, an optionally substituted 8-10 membered saturated, partially unsaturated, or aryl bicyclic ring having 0-4 heteroatoms independently selected from nitrogen, oxygen, or sulfur, a detectable moiety, a polymer residue, a peptide, a sugar-containing or sugar-like moiety, or:

wherein when R¹⁰ is a ring, R¹⁰ is optionally substituted at any substitutable carbon with 1-5 R¹¹ and at any substitutable nitrogen with R¹²;

each R¹¹ is independently halogen, R, OR, SR, N(R)₂, N(R)C(O)R, N(R)C(O)OR, N(R)C(O)N(R)₂, N(R)SO₂R, N(R)SO₂OR, S(O)R, SO₂R, OSO₂R, C(O)R, CO₂R, OCO₂R, C(O)N(R)₂, or OC(O)N(R)₂, or wherein:

two R¹¹ are optionally taken together to form an oxo moiety or an optionally substituted 3-8 membered saturated or partially unsaturated fused or spirofused ring having 0-4 heteroatoms independently selected from nitrogen, oxygen, or sulfur; and

each R¹² is independently R, OR, S(O)R, SO₂R, OSO₂R, C(O)R, CO₂R, OCO₂R, C(O)N(R)₂, or OC(O)N(R)₂, or wherein:

R¹² and R¹¹ are optionally taken together to form an optionally substituted 3-8 membered saturated or partially unsaturated fused ring having 0-4 heteroatoms independently selected from nitrogen, oxygen, or sulfur.

In certain embodiments, the present invention provides a compound wherein Ring A is a 7 membered saturated carbocycle, wherein Q is an optionally substituted C₁₋₁₀ alkylene chain wherein one, two, or three methylene units of Q are optionally and independently replaced by —O—, —N(R)—, —S—, or —Cy-, and wherein R¹⁰ of the Q-R¹⁰ moiety is selected from the group consisting of hydrogen, halogen, a suitably protected hydroxyl group, a suitably protected thiol group, or a suitably protected amino group.

In certain embodiments, the present invention provides a compound wherein Ring A is a 7 membered saturated carbocycle, wherein Q is an optionally substituted C₁₋₁₀ alkylene chain wherein one, two, or three methylene units of Q are optionally and independently replaced by —O—, —N(R)—, —S—, or —Cy-, and wherein R¹⁰ of the Q-R¹⁰ moiety is a ring optionally substituted at any substitutable carbon with 1-5 R¹¹ and at any substitutable nitrogen with R¹², wherein each R¹¹ is independently selected from halogen, R, OR, SR, N(R)₂, N(R)C(O)R, N(R)C(O)OR, N(R)C(O)N(R)₂, N(R)SO₂R, N(R)SO₂OR, S(O)R, SO₂R, OSO₂R, C(O)R, CO₂R, OCO₂R, C(O)N(R)₂, or OC(O)N(R)₂. In certain embodiments, the compound is as described above and R¹⁰ is an optionally substituted heterocycle. In certain embodiments, the compound is as described above and R¹⁰ is an optionally substituted 5-6 membered heterocycle with 1-3 heteroatoms. In certain embodiments, the compound is as described above and R¹⁰ is an optionally substituted 6 membered heterocycle with 2 heteroatoms. In certain embodiments, the compound is as described above and R¹⁰ is optionally substituted morpholine.

In certain embodiments, the present invention provides a compound wherein Ring A is a 7 membered saturated carbocycle, wherein Q is an optionally substituted C₁₋₁₀ alkylene chain wherein one, two, or three methylene units of Q are optionally and independently replaced by —O—, —N(R)—, —S—, or —Cy-, and wherein R¹⁰ of the Q-R¹⁰ moiety is a sugar-containing or sugar-like moiety.

In certain embodiments, the present invention provides a compound wherein Ring A is a 7 membered partially unsaturated carbocycle, wherein Q is an optionally substituted C₁₋₁₀ alkylene chain wherein one, two, or three methylene units of Q are optionally and independently replaced by —O—, —N(R)—, —S—, or —Cy-, and wherein R¹⁰ of the Q-R¹⁰ moiety is selected from the group consisting of hydrogen, halogen, a suitably protected hydroxyl group, a suitably protected thiol group, or a suitably protected amino group.

In certain embodiments, the present invention provides a compound wherein Ring A is a 7 membered partially unsaturated carbocycle, wherein Q is an optionally substituted C₁₋₁₀ alkylene chain wherein one, two, or three methylene units of Q are optionally and independently replaced by —O—, —N(R)—, —S—, or —Cy-, and wherein R¹⁰ of the Q-R¹⁰ moiety is a ring optionally substituted at any substitutable carbon with 1-5 R¹¹ and at any substitutable nitrogen with R¹², wherein each R¹¹ is independently selected from halogen, R, OR, SR, N(R)₂, N(R)C(O)R, N(R)C(O)OR, N(R)C(O)N(R)₂, N(R)SO₂R, N(R)SO₂OR, S(O)R, SO₂R, OSO₂R, C(O)R, CO₂R, OCO₂R, C(O)N(R)₂, or OC(O)N(R)₂. In certain embodiments, the compound is as described above and R¹⁰ is an optionally substituted heterocycle. In certain embodiments, the compound is as described above and R¹⁰ is an optionally substituted 5-6 membered heterocycle with 1-3 heteroatoms. In certain embodiments, the compound is as described above and R¹⁰ is an optionally substituted 6 membered heterocycle with 2 heteroatoms. In certain embodiments, the compound is as described above and R¹⁰ is optionally substituted morpholine.

In certain embodiments, the present invention provides a compound wherein Ring A is a 7 membered partially unsaturated carbocycle, wherein Q is an optionally substituted C₁₋₁₀ alkylene chain wherein one, two, or three methylene units of Q are optionally and independently replaced by —O—, —N(R)—, —S—, or —Cy-, and wherein R¹⁰ of the Q-R¹⁰ moiety is a sugar-containing or sugar-like moiety.

In some embodiments, the present invention provides a compound wherein Ring A is a 7 membered saturated or partially unsaturated ring having 1-2 heteroatoms independently selected from nitrogen, oxygen, or sulfur, and wherein Q are optionally and independently replaced by —O—, —N(R)—, —S—, —C(O)—, —OC(O)—, —C(O)O—, —OC(O)O—, —S(O)—, or —S(O)₂—, —OSO₂O—, —N(R)C(O)—, —C(O)NR—, —N(R)C(O)O—, —OC(O)NR—, —N(R)C(O)NR—, or —Cy-, wherein: each —Cy- is independently a bivalent optionally substituted saturated, partially unsaturated, or aromatic monocyclic or bicyclic ring selected from a 6-10 membered arylene, a 5-10 membered heteroarylene having 1-4 heteroatoms independently selected from oxygen, nitrogen, or sulfur, a 3-8 membered carbocyclylene, or a 3-10 membered heterocyclylene having 1-4 heteroatoms independently selected from oxygen, nitrogen, or sulfur, and:

wherein R¹⁰ of the Q-R¹⁰ moiety is selected from the group consisting of hydrogen, halogen, an optionally substituted C₁₋₁₀ aliphatic, a suitably protected hydroxyl group, a suitably protected thiol group, a suitably protected amino group, an optionally substituted 3-8 membered saturated, partially unsaturated, or aryl monocyclic ring having 0-4 heteroatoms independently selected from nitrogen, oxygen, or sulfur, an optionally substituted 8-10 membered saturated, partially unsaturated, or aryl bicyclic ring having 0-4 heteroatoms independently selected from nitrogen, oxygen, or sulfur, a detectable moiety, a polymer residue, a peptide, a sugar-containing or sugar-like moiety, or:

wherein when R¹⁰ is a ring, R¹⁰ is optionally substituted at any substitutable carbon with 1-5 R¹¹ and at any substitutable nitrogen with R¹²;

each R¹¹ is independently halogen, R, OR, SR, N(R)₂, N(R)C(O)R, N(R)C(O)OR, N(R)C(O)N(R)₂, N(R)SO₂R, N(R)SO₂OR, S(O)R, SO₂R, OSO₂R, C(O)R, CO₂R, OCO₂R, C(O)N(R)₂, or OC(O)N(R)₂, or wherein:

two R¹¹ are optionally taken together to form an oxo moiety or an optionally substituted 3-8 membered saturated or partially unsaturated fused or spirofused ring having 0-4 heteroatoms independently selected from nitrogen, oxygen, or sulfur; and

each R¹² is independently R, OR, S(O)R, SO₂R, OSO₂R, C(O)R, CO₂R, OCO₂R, C(O)N(R)₂, or OC(O)N(R)₂, or wherein:

R¹² and R¹¹ are optionally taken together to form an optionally substituted 3-8 membered saturated or partially unsaturated fused ring having 0-4 heteroatoms independently selected from nitrogen, oxygen, or sulfur.

In certain embodiments, the present invention provides a compound wherein Ring A is a 7 membered saturated ring having 1-2 heteroatoms independently selected from nitrogen, oxygen, or sulfur, wherein Q is an optionally substituted C₁₋₁₀ alkylene chain wherein one, two, or three methylene units of Q are optionally and independently replaced by —O—, —N(R)—, —S—, or —Cy-, and wherein R¹⁰ of the Q-R¹⁰ moiety is selected from the group consisting of hydrogen, halogen, a suitably protected hydroxyl group, a suitably protected thiol group, or a suitably protected amino group.

In certain embodiments, the present invention provides a compound wherein Ring A is a 7 membered saturated ring having 1-2 heteroatoms independently selected from nitrogen, oxygen, or sulfur, wherein Q is an optionally substituted C₁₋₁₀ alkylene chain wherein one, two, or three methylene units of Q are optionally and independently replaced by —O—, —N(R)—, —S—, or —Cy-, and wherein R¹⁰ of the Q-R¹⁰ moiety is a ring optionally substituted at any substitutable carbon with 1-5 R¹¹ and at any substitutable nitrogen with R¹², wherein each R¹¹ is independently selected from halogen, R, OR, SR, N(R)₂, N(R)C(O)R, N(R)C(O)OR, N(R)C(O)N(R)₂, N(R)SO₂R, N(R)SO₂OR, S(O)R, SO₂R, OSO₂R, C(O)R, CO₂R, OCO₂R, C(O)N(R)₂, or OC(O)N(R)₂. In certain embodiments, the compound is as described above and R¹⁰ is an optionally substituted heterocycle. In certain embodiments, the compound is as described above and R¹⁰ is an optionally substituted 5-6 membered heterocycle with 1-3 heteroatoms. In certain embodiments, the compound is as described above and R¹⁰ is an optionally substituted 6 membered heterocycle with 2 heteroatoms. In certain embodiments, the compound is as described above and R¹⁰ is optionally substituted morpholine.

In certain embodiments, the present invention provides a compound wherein Ring A is a 7 membered saturated ring having 1-2 heteroatoms independently selected from nitrogen, oxygen, or sulfur, wherein Q is an optionally substituted C₁₋₁₀ alkylene chain wherein one, two, or three methylene units of Q are optionally and independently replaced by —O—, —N(R)—, —S—, or —Cy-, and wherein R¹⁰ of the Q-R¹⁰ moiety is a sugar-containing or sugar-like moiety.

In certain embodiments, the present invention provides a compound wherein Ring A is a 7 membered partially unsaturated ring having 1-2 heteroatoms independently selected from nitrogen, oxygen, or sulfur, wherein Q is an optionally substituted C₁₋₁₀ alkylene chain wherein one, two, or three methylene units of Q are optionally and independently replaced by —O—, —N(R)—, —S—, or —Cy-, and wherein R¹⁰ of the Q-R¹⁰ moiety is selected from the group consisting of hydrogen, halogen, a suitably protected hydroxyl group, a suitably protected thiol group, or a suitably protected amino group.

In certain embodiments, the present invention provides a compound wherein Ring A is a 7 membered partially unsaturated ring having 1-2 heteroatoms independently selected from nitrogen, oxygen, or sulfur, wherein Q is an optionally substituted C₁₋₁₀ alkylene chain wherein one, two, or three methylene units of Q are optionally and independently replaced by —O—, —N(R)—, —S—, or —Cy-, and wherein R¹⁰ of the Q-R¹⁰ moiety is a ring optionally substituted at any substitutable carbon with 1-5 R¹¹ and at any substitutable nitrogen with R¹², wherein each R¹¹ is independently selected from halogen, R, OR, SR, N(R)₂, N(R)C(O)R, N(R)C(O)OR, N(R)C(O)N(R)₂, N(R)SO₂R, N(R)SO₂OR, S(O)R, SO₂R, OSO₂R, C(O)R, CO₂R, OCO₂R, C(O)N(R)₂, or OC(O)N(R)₂. In certain embodiments, the compound is as described above and R¹⁰ is an optionally substituted heterocycle. In certain embodiments, the compound is as described above and R¹⁰ is an optionally substituted 5-6 membered heterocycle with 1-3 heteroatoms. In certain embodiments, the compound is as described above and R¹⁰ is an optionally substituted 6 membered heterocycle with 2 heteroatoms. In certain embodiments, the compound is as described above and R¹⁰ is optionally substituted morpholine.

In certain embodiments, the present invention provides a compound wherein Ring A is a 7 membered partially unsaturated ring having 1-2 heteroatoms independently selected from nitrogen, oxygen, or sulfur, wherein Q is an optionally substituted C₁₋₁₀ alkylene chain wherein one, two, or three methylene units of Q are optionally and independently replaced by —O—, —N(R)—, —S—, or —Cy-, and wherein R¹⁰ of the Q-R¹⁰ moiety is a sugar-containing or sugar-like moiety.

### Exemplary Ring A/Ring D Combinations

In some embodiments, the present invention provides a compound wherein Ring A is a 5 membered saturated or partially unsaturated carbocycle, and wherein R³ and R⁸ of Ring D are each independently selected from halogen, CN, R, OR, a suitably protected hydroxyl group, SR, a suitably protected thiol group, S(O)R, SO₂R, OSO₂R, N(R)₂, a suitably protected amino group, N(R)C(O)R, N(R)C(O)C(O)R, N(R)C(O)N(R)₂, N(R)C(O)OR, C(O)OR, OC(O)R, C(O)N(R)₂, or OC(O)N(R)₂, and wherein R⁷ and R^{7′} of Ring D are each independently selected from halogen, CN, N₃, R, OR, a suitably protected hydroxyl group, SR, a suitably protected thiol group, SO₂R, OSO₂R, N(R)₂, a suitably protected amino group, N(R)C(O)R, N(R)C(O)C(O)R, N(R)C(O)N(R)₂, N(R)C(O)OR, C(O)OR, OC(O)R, C(O)N(R)₂, or OC(O)N(R)₂, or:

R⁷ and R^{7′} are taken together to form an oxo moiety or an optionally substituted 3-8 membered saturated or partially unsaturated spirocycle having 0-4 heteroatoms independently selected from nitrogen, oxygen, or sulfur.

In some embodiments, the present invention provides a compound wherein Ring A is a 5 membered saturated carbocycle, and wherein R³ and R⁸ of Ring D are each independently selected from halogen, R, OR, or a suitably protected hydroxyl group, and wherein R⁷ and R^{7′} of Ring D are each independently selected from halogen, R, OR, a suitably protected hydroxyl group, or R⁷ and R^{7′} are taken together to form an oxo moiety.

In some embodiments, the present invention provides a compound wherein Ring A is a 5 membered partially unsaturated carbocycle, and wherein R³ and R⁸ of Ring D are each independently selected from halogen, R, OR, or a suitably protected hydroxyl group, and wherein R⁷ and R^{7′} of Ring D are each independently selected from halogen, R, OR, a suitably protected hydroxyl group, or R⁷ and R^{7′} are taken together to form an oxo moiety.

In some embodiments, the present invention provides a compound wherein Ring A is a 5 membered saturated or partially unsaturated ring having 1-2 heteroatoms independently selected from nitrogen, oxygen, or sulfur, and wherein R³ and R⁸ of Ring D are each independently selected from halogen, CN, R, OR, a suitably protected hydroxyl group, SR, a suitably protected thiol group, S(O)R, SO₂R, OSO₂R, N(R)₂, a suitably protected amino group, N(R)C(O)R, N(R)C(O)C(O)R, N(R)C(O)N(R)₂, N(R)C(O)OR, C(O)OR, OC(O)R, C(O)N(R)₂, or OC(O)N(R)₂, and wherein R⁷ and R^{7′} of Ring D are each independently selected from halogen, CN, N₃, R, OR, a suitably protected hydroxyl group, SR, a suitably protected thiol group, SO₂R, OSO₂R, N(R)₂, a suitably protected amino group, N(R)C(O)R, N(R)C(O)C(O)R, N(R)C(O)N(R)₂, N(R)C(O)OR, C(O)OR, OC(O)R, C(O)N(R)₂, OC(O)N(R)₂, OC(O)N(R)₂, or:

R⁷ and R^{7′} are taken together to form an oxo moiety or an optionally substituted 3-8 membered saturated or partially unsaturated spirocycle having 0-4 heteroatoms independently selected from nitrogen, oxygen, or sulfur.

In some embodiments, the present invention provides a compound wherein Ring A is a 5 membered saturated ring having 1-2 heteroatoms independently selected from nitrogen, oxygen, or sulfur, and wherein R³ and R⁸ of Ring D are each independently selected from halogen, R, OR, or a suitably protected hydroxyl group, and wherein R⁷ and R^{7′} of Ring D are each independently selected from halogen, R, OR, a suitably protected hydroxyl group, or R⁷ and R^{7′} are taken together to form an oxo moiety.

In some embodiments, the present invention provides a compound wherein Ring A is a 5 membered partially unsaturated ring having 1-2 heteroatoms independently selected from nitrogen, oxygen, or sulfur, and wherein R³ and R⁸ of Ring D are each independently selected from halogen, R, OR, or a suitably protected hydroxyl group, and wherein R⁷ and R^{7′} of Ring D are each independently selected from halogen, R, OR, a suitably protected hydroxyl group, OC(O)N(R)₂, or:

R⁷ and R^{7′} are taken together to form an oxo moiety or an optionally substituted 3-8 membered saturated or partially unsaturated spirocycle having 0-4 heteroatoms independently selected from nitrogen, oxygen, or sulfur.

In some embodiments, the present invention provides a compound wherein Ring A is a 6 membered saturated or partially unsaturated carbocycle, and wherein R³ and R⁸ of Ring D are each independently selected from halogen, CN, R, OR, a suitably protected hydroxyl group, SR, a suitably protected thiol group, S(O)R, SO₂R, OSO₂R, N(R)₂, a suitably protected amino group, N(R)C(O)R, N(R)C(O)C(O)R, N(R)C(O)N(R)₂, N(R)C(O)OR, C(O)OR, OC(O)R, C(O)N(R)₂, or OC(O)N(R)₂, and wherein R⁷ and R^{7′} of Ring D are each independently selected from halogen, CN, N₃, R, OR, a suitably protected hydroxyl group, SR, a suitably protected thiol group, SO₂R, OSO₂R, N(R)₂, a suitably protected amino group, N(R)C(O)R, N(R)C(O)C(O)R, N(R)C(O)N(R)₂, N(R)C(O)OR, C(O)OR, OC(O)R, C(O)N(R)₂, OC(O)N(R)₂, or:

R⁷ and R^{7′} are taken together to form an oxo moiety or an optionally substituted 3-8 membered saturated or partially unsaturated spirocycle having 0-4 heteroatoms independently selected from nitrogen, oxygen, or sulfur.

In some embodiments, the present invention provides a compound wherein Ring A is a 6 membered saturated carbocycle, and wherein R³ and R⁸ of Ring D are each independently selected from halogen, R, OR, or a suitably protected hydroxyl group, and wherein R⁷ and R^{7′} of Ring D are each independently selected from halogen, R, OR, a suitably protected hydroxyl group, or R⁷ and R^{7′} are taken together to form an oxo moiety.

In some embodiments, the present invention provides a compound wherein Ring A is a 6 membered partially unsaturated carbocycle, and wherein R³ and R⁸ of Ring D are each independently selected from halogen, R, OR, or a suitably protected hydroxyl group, and wherein R⁷ and R^{7′} of Ring D are each independently selected from halogen, R, OR, a suitably protected hydroxyl group, or R⁷ and R^{7′} are taken together to form an oxo moiety.

In some embodiments, the present invention provides a compound wherein Ring A is a 6 membered saturated or partially unsaturated ring having 1-2 heteroatoms independently selected from nitrogen, oxygen, or sulfur, and wherein R³ and R⁸ of Ring D are each independently selected from halogen, CN, R, OR, a suitably protected hydroxyl group, SR, a suitably protected thiol group, S(O)R, SO₂R, OSO₂R, N(R)₂, a suitably protected amino group, N(R)C(O)R, N(R)C(O)C(O)R, N(R)C(O)N(R)₂, N(R)C(O)OR, C(O)OR, OC(O)R, C(O)N(R)₂, or OC(O)N(R)₂, and wherein R⁷ and R^{7′} of Ring D are each independently selected from halogen, CN, N₃, R, OR, a suitably protected hydroxyl group, SR, a suitably protected thiol group, SO₂R, OSO₂R, N(R)₂, a suitably protected amino group, N(R)C(O)R, N(R)C(O)C(O)R, N(R)C(O)N(R)₂, N(R)C(O)OR, C(O)OR, OC(O)R, C(O)N(R)₂, OC(O)N(R)₂, or:

R⁷ and R^{7′} are taken together to form an oxo moiety or an optionally substituted 3-8 membered saturated or partially unsaturated spirocycle having 0-4 heteroatoms independently selected from nitrogen, oxygen, or sulfur.

In some embodiments, the present invention provides a compound wherein Ring A is a 6 membered saturated ring having 1-2 heteroatoms independently selected from nitrogen, oxygen, or sulfur, and wherein R³ and R⁸ of Ring D are each independently selected from halogen, R, OR, or a suitably protected hydroxyl group, and wherein R⁷ and R^{7′} of Ring D are each independently selected from halogen, R, OR, a suitably protected hydroxyl group, or R⁷ and R^{7′} are taken together to form an oxo moiety.

In some embodiments, the present invention provides a compound wherein Ring A is a 6 membered partially unsaturated ring having 1-2 heteroatoms independently selected from nitrogen, oxygen, or sulfur, and wherein R³ and R⁸ of Ring D are each independently selected from halogen, R, OR, or a suitably protected hydroxyl group, and wherein R⁷ and R^{7′} of Ring D are each independently selected from halogen, R, OR, a suitably protected hydroxyl group, or R⁷ and R^{7′} are taken together to form an oxo moiety.

In some embodiments, the present invention provides a compound wherein Ring A is a 7 membered saturated or partially unsaturated carbocycle, and wherein R³ and R⁸ of Ring D are each independently selected from halogen, CN, R, OR, a suitably protected hydroxyl group, SR, a suitably protected thiol group, S(O)R, SO₂R, OSO₂R, N(R)₂, a suitably protected amino group, N(R)C(O)R, N(R)C(O)C(O)R, N(R)C(O)N(R)₂, N(R)C(O)OR, C(O)OR, OC(O)R, C(O)N(R)₂, or OC(O)N(R)₂, and wherein R⁷ and R^{7′} of Ring D are each independently selected from halogen, CN, N₃, R, OR, a suitably protected hydroxyl group, SR, a suitably protected thiol group, SO₂R, OSO₂R, N(R)₂, a suitably protected amino group, N(R)C(O)R, N(R)C(O)C(O)R, N(R)C(O)N(R)₂, N(R)C(O)OR, C(O)OR, OC(O)R, C(O)N(R)₂, OC(O)N(R)₂, or:

R⁷ and R^{7′} are taken together to form an oxo moiety or an optionally substituted 3-8 membered saturated or partially unsaturated spirocycle having 0-4 heteroatoms independently selected from nitrogen, oxygen, or sulfur.

In some embodiments, the present invention provides a compound wherein Ring A is a 7 membered saturated carbocycle, and wherein R³ and R⁸ of Ring D are each independently selected from halogen, R, OR, or a suitably protected hydroxyl group, and wherein R⁷ and R^{7′} of Ring D are each independently selected from halogen, R, OR, a suitably protected hydroxyl group, or R⁷ and R^{7′} are taken together to form an oxo moiety.

In some embodiments, the present invention provides a compound wherein Ring A is a 7 membered partially unsaturated carbocycle, and wherein R³ and R⁸ of Ring D are each independently selected from halogen, R, OR, or a suitably protected hydroxyl group, and wherein R⁷ and R^{7′} of Ring D are each independently selected from halogen, R, OR, a suitably protected hydroxyl group, or R⁷ and R^{7′} are taken together to form an oxo moiety.

In some embodiments, the present invention provides a compound wherein Ring A is a 7 membered saturated or partially unsaturated ring having 1-2 heteroatoms independently selected from nitrogen, oxygen, or sulfur, and wherein R³ and R⁸ of Ring D are each independently selected from halogen, CN, R, OR, a suitably protected hydroxyl group, SR, a suitably protected thiol group, S(O)R, SO₂R, OSO₂R, N(R)₂, a suitably protected amino group, N(R)C(O)R, N(R)C(O)C(O)R, N(R)C(O)N(R)₂, N(R)C(O)OR, C(O)OR, OC(O)R, C(O)N(R)₂, or OC(O)N(R)₂, and wherein R⁷ and R^{7′} of Ring D are each independently selected from halogen, CN, N₃, R, OR, a suitably protected hydroxyl group, SR, a suitably protected thiol group, SO₂R, OSO₂R, N(R)₂, a suitably protected amino group, N(R)C(O)R, N(R)C(O)C(O)R, N(R)C(O)N(R)₂, N(R)C(O)OR, C(O)OR, OC(O)R, C(O)N(R)₂, OC(O)N(R)₂, or:

R⁷ and R^{7′} are taken together to form an oxo moiety or an optionally substituted 3-8 membered saturated or partially unsaturated spirocycle having 0-4 heteroatoms independently selected from nitrogen, oxygen, or sulfur.

In some embodiments, the present invention provides a compound wherein Ring A is a 7 membered saturated ring having 1-2 heteroatoms independently selected from nitrogen, oxygen, or sulfur, and wherein R³ and R⁸ of Ring D are each independently selected from halogen, R, OR, or a suitably protected hydroxyl group, and wherein R⁷ and R^{7′} of Ring D are each independently selected from halogen, R, OR, a suitably protected hydroxyl group, or R⁷ and R^{7′} are taken together to form an oxo moiety.

In some embodiments, the present invention provides a compound wherein Ring A is a 7 membered partially unsaturated ring having 1-2 heteroatoms independently selected from nitrogen, oxygen, or sulfur, and wherein R³ and R⁸ of Ring D are each independently selected from halogen, R, OR, or a suitably protected hydroxyl group, and wherein R⁷ and R^{7′} of Ring D are each independently selected from halogen, R, OR, a suitably protected hydroxyl group, or R⁷ and R^{7′} are taken together to form an oxo moiety.

### Exemplary Ring A/Ring E Combinations

In some embodiments, the present invention provides a compound wherein Ring A is a 5 membered saturated or partially unsaturated carbocycle, and wherein Ring E is a 4-7 membered saturated, partially unsaturated, or aromatic ring having 0-2 heteroatoms independently selected from nitrogen, oxygen, or sulfur, and wherein each R⁴ of Ring E is independently selected from halogen, CN, R, OR, a suitably protected hydroxyl group, SR, a suitably protected thiol group, S(O)R, SO₂R, OSO₂R, N(R)₂, a suitably protected amino group, N(R)C(O)R, N(R)C(O)C(O)R, N(R)C(O)N(R)₂, N(R)C(O)OR, C(O)OR, OC(O)R, C(O)N(R)₂, or OC(O)N(R)₂, or:

two R⁴ on the same carbon are optionally taken together to form an optionally substituted 3-8 membered saturated or partially unsaturated spirofused ring having 0-4 heteroatoms independently selected from nitrogen, oxygen, or sulfur, or:

two R⁴ on the same carbon are optionally taken together to form an oxo moiety, an oxime, an optionally substituted hydrazone, or an optionally substituted imine;

and wherein each R⁵ of Ring E is independently T-C(R′)₃, T-C(R′)₂C(R″)₃, OR, a suitably protected hydroxyl group, SR, a suitably protected thiol group, S(O)R, SO₂R, OSO₂R, N(R)₂, a suitably protected amino group, N(R)C(O)R, N(R)C(O)C(O)R, N(R)C(O)N(R)₂, N(R)C(O)OR, C(O)OR, OC(O)R, C(O)N(R)₂, or OC(O)N(R)₂, an optionally substituted 3-8 membered saturated, partially unsaturated, or aryl monocyclic ring having 0-4 heteroatoms independently selected from nitrogen, oxygen, or sulfur, an optionally substituted 8-10 membered saturated, partially unsaturated, or aryl bicyclic ring having 0-4 heteroatoms independently selected from nitrogen, oxygen, or sulfur, or:

two R⁵ on the same carbon are optionally taken together to form an oxo moiety, an oxime, an optionally substituted hydrazone, or an optionally substituted imine;

each T is independently a valence bond or an optionally substituted straight or branched, saturated or unsaturated, C₁₋₆ alkylene chain wherein up to two methylene units of T are optionally and independently replaced by —O—, —N(R)—, —S—, —C(O)—, —S(O)—, or —S(O)₂—;

each R′ and R″ is independently selected from halogen, R, OR, SR, S(O)R, SO₂R, OSO₂R, N(R)₂, N(R)C(O)R, N(R)C(O)C(O)R, N(R)C(O)N(R)₂, N(R)C(O)OR, N(R)S(O)R, N(R)SO₂R, N(R)SO₂OR C(O)OR, OC(O)R, C(O)N(R)₂, OC(O)N(R)₂, or an optionally substituted 3-8 membered saturated, partially unsaturated, or aryl monocyclic ring having 0-4 heteroatoms independently selected from nitrogen, oxygen, or sulfur, or an optionally substituted 8-10 membered saturated, partially unsaturated, or aryl bicyclic ring having 0-4 heteroatoms independently selected from nitrogen, oxygen, or sulfur, or:

two R′ are optionally taken together to form an oxo moiety, an oxime, an optionally substituted hydrazone, an optionally substituted imine, or an optionally substituted 3-8 membered saturated or partially unsaturated ring having 0-4 heteroatoms independently selected from nitrogen, oxygen, or sulfur, or:

two R″ are optionally taken together to form an oxo moiety, an oxime, an optionally substituted hydrazone, an optionally substituted imine, or an optionally substituted 3-8 membered saturated or partially unsaturated ring having 0-4 heteroatoms independently selected from nitrogen, oxygen, or sulfur; or

R⁶ and R⁵ are optionally taken together to form an optionally substituted 3-8 membered saturated, partially unsaturated, or aryl ring having 0-4 heteroatoms independently selected from nitrogen, oxygen, or sulfur.

In certain embodiments, the present invention provides a compound wherein Ring A is a 5 membered saturated or partially unsaturated carbocycle, and wherein Ring E is a 4 membered saturated ring having 1-2 heteroatoms independently selected from nitrogen, oxygen, or sulfur, and wherein each R⁴ and R⁵ of Ring E is independently as described above.

In certain embodiments, the present invention provides a compound wherein Ring A is a 5 membered saturated or partially unsaturated carbocycle, and wherein Ring E is a 4 membered saturated carbocycle, wherein each R⁴ and R⁵ of Ring E is independently as described above.

In certain embodiments, the present invention provides a compound wherein Ring A is a 5 membered saturated or partially unsaturated carbocycle, and wherein Ring E is a 5 membered saturated, partially unsaturated, or aromatic ring having 1-2 heteroatoms independently selected from nitrogen, oxygen, or sulfur, and wherein each R⁴ and R⁵ of Ring E is independently as described above.

In certain embodiments, the present invention provides a compound wherein Ring A is a 5 membered saturated or partially unsaturated carbocycle, and wherein Ring E is a 5 membered saturated or partially unsaturated carbocycle, wherein each R⁴ and R⁵ of Ring E is independently as described above.

In certain embodiments, the present invention provides a compound wherein Ring A is a 5 membered saturated or partially unsaturated carbocycle, and wherein Ring E is a 6 membered saturated, partially unsaturated, or aromatic ring having 1-2 heteroatoms independently selected from nitrogen, oxygen, or sulfur, and wherein each R⁴ and R⁵ of Ring E is independently as described above.

In certain embodiments, the present invention provides a compound wherein Ring A is a 5 membered saturated or partially unsaturated carbocycle, and wherein Ring E is a 6 membered saturated, partially unsaturated, or aromatic carbocycle, and wherein each R⁴ and R⁵ of Ring E is independently as described above.

In certain embodiments, the present invention provides a compound wherein Ring A is a 5 membered saturated or partially unsaturated carbocycle, and wherein Ring E is a 7 membered saturated or partially unsaturated ring having 1-2 heteroatoms independently selected from nitrogen, oxygen, or sulfur, and wherein each R⁴ and R⁵ of Ring E is independently as described above.

In certain embodiments, the present invention provides a compound wherein Ring A is a 5 membered saturated or partially unsaturated carbocycle, and wherein Ring E is a 7 membered saturated or partially unsaturated carbocycle, wherein each R⁴ and R⁵ of Ring E is independently as described above.

In some embodiments, the present invention provides a compound wherein Ring A is a 5 membered saturated or partially unsaturated ring having 1-2 heteroatoms independently selected from nitrogen, oxygen, or sulfur, and wherein Ring E is a 4-7 membered saturated, partially unsaturated, or aromatic ring having 0-2 heteroatoms independently selected from nitrogen, oxygen, or sulfur, and wherein each R⁴ of Ring E is independently selected from halogen, CN, R, OR, a suitably protected hydroxyl group, SR, a suitably protected thiol group, S(O)R, SO₂R, OSO₂R, N(R)₂, a suitably protected amino group, N(R)C(O)R, N(R)C(O)C(O)R, N(R)C(O)N(R)₂, N(R)C(O)OR, C(O)OR, OC(O)R, C(O)N(R)₂, or OC(O)N(R)₂, or:

two R⁴ on the same carbon are optionally taken together to form an optionally substituted 3-8 membered saturated or partially unsaturated spirofused ring having 0-4 heteroatoms independently selected from nitrogen, oxygen, or sulfur, or:

two R⁴ on the same carbon are optionally taken together to form an oxo moiety, an oxime, an optionally substituted hydrazone, or an optionally substituted imine;

and wherein each R⁵ of Ring E is independently T-C(R′)₃, T-C(R′)₂C(R″)₃, OR, a suitably protected hydroxyl group, SR, a suitably protected thiol group, S(O)R, SO₂R, OSO₂R, N(R)₂, a suitably protected amino group, N(R)C(O)R, N(R)C(O)C(O)R, N(R)C(O)N(R)₂, N(R)C(O)OR, C(O)OR, OC(O)R, C(O)N(R)₂, or OC(O)N(R)₂, an optionally substituted 3-8 membered saturated, partially unsaturated, or aryl monocyclic ring having 0-4 heteroatoms independently selected from nitrogen, oxygen, or sulfur, an optionally substituted 8-10 membered saturated, partially unsaturated, or aryl bicyclic ring having 0-4 heteroatoms independently selected from nitrogen, oxygen, or sulfur, or:

two R⁵ on the same carbon are optionally taken together to form an oxo moiety, an oxime, an optionally substituted hydrazone, or an optionally substituted imine;

each T is independently a valence bond or an optionally substituted straight or branched, saturated or unsaturated, C₁₋₆ alkylene chain wherein up to two methylene units of T are optionally and independently replaced by —O—, —N(R)—, —S—, —C(O)—, —S(O)—, or —S(O)₂—;

each R′ and R″ is independently selected from halogen, R, OR, SR, S(O)R, SO₂R, OSO₂R, N(R)₂, N(R)C(O)R, N(R)C(O)C(O)R, N(R)C(O)N(R)₂, N(R)C(O)OR, N(R)S(O)R, N(R)SO₂R, N(R)SO₂OR C(O)OR, OC(O)R, C(O)N(R)₂, OC(O)N(R)₂, or an optionally substituted 3-8 membered saturated, partially unsaturated, or aryl monocyclic ring having 0-4 heteroatoms independently selected from nitrogen, oxygen, or sulfur, or an optionally substituted 8-10 membered saturated, partially unsaturated, or aryl bicyclic ring having 0-4 heteroatoms independently selected from nitrogen, oxygen, or sulfur, or:

two R′ are optionally taken together to form an oxo moiety, an oxime, an optionally substituted hydrazone, an optionally substituted imine, or an optionally substituted 3-8 membered saturated or partially unsaturated ring having 0-4 heteroatoms independently selected from nitrogen, oxygen, or sulfur, or:

two R″ are optionally taken together to form an oxo moiety, an oxime, an optionally substituted hydrazone, an optionally substituted imine, or an optionally substituted 3-8 membered saturated or partially unsaturated ring having 0-4 heteroatoms independently selected from nitrogen, oxygen, or sulfur; or

R⁶ and R⁵ are optionally taken together to form an optionally substituted 3-8 membered saturated, partially unsaturated, or aryl ring having 0-4 heteroatoms independently selected from nitrogen, oxygen, or sulfur.

In certain embodiments, the present invention provides a compound wherein Ring A is a 5 membered saturated or partially unsaturated ring having 1-2 heteroatoms independently selected from nitrogen, oxygen, or sulfur, and wherein Ring E is a 4 membered saturated ring having 1-2 heteroatoms independently selected from nitrogen, oxygen, or sulfur, and wherein each R⁴ and R⁵ of Ring E is independently as described above.

In certain embodiments, the present invention provides a compound wherein Ring A is a 5 membered saturated or partially unsaturated ring having 1-2 heteroatoms independently selected from nitrogen, oxygen, or sulfur, and wherein Ring E is a 4 membered saturated carbocycle, wherein each R⁴ and R⁵ of Ring E is independently as described above.

In certain embodiments, the present invention provides a compound wherein Ring A is a 5 membered saturated or partially unsaturated ring having 1-2 heteroatoms independently selected from nitrogen, oxygen, or sulfur, and wherein Ring E is a 5 membered saturated, partially unsaturated, or aromatic ring having 1-2 heteroatoms independently selected from nitrogen, oxygen, or sulfur, and wherein each R⁴ and R⁵ of Ring E is independently as described above.

In certain embodiments, the present invention provides a compound wherein Ring A is a 5 membered saturated or partially unsaturated ring having 1-2 heteroatoms independently selected from nitrogen, oxygen, or sulfur, and wherein Ring E is a 5 membered saturated or partially unsaturated carbocycle, wherein each R⁴ and R⁵ of Ring E is independently as described above.

In certain embodiments, the present invention provides a compound wherein Ring A is a 5 membered saturated or partially unsaturated ring having 1-2 heteroatoms independently selected from nitrogen, oxygen, or sulfur, and wherein Ring E is a 6 membered saturated, partially unsaturated, or aromatic ring having 1-2 heteroatoms independently selected from nitrogen, oxygen, or sulfur, and wherein each R⁴ and R⁵ of Ring E is independently as described above.

In certain embodiments, the present invention provides a compound wherein Ring A is a 5 membered saturated or partially unsaturated ring having 1-2 heteroatoms independently selected from nitrogen, oxygen, or sulfur, and wherein Ring E is a 6 membered saturated, partially unsaturated, or aromatic carbocycle, wherein each R⁴ and R⁵ of Ring E is independently as described above.

In certain embodiments, the present invention provides a compound wherein Ring A is a 5 membered saturated or partially unsaturated ring having 1-2 heteroatoms independently selected from nitrogen, oxygen, or sulfur, and wherein Ring E is a 7 membered saturated or partially unsaturated ring having 1-2 heteroatoms independently selected from nitrogen, oxygen, or sulfur, and wherein each R⁴ and R⁵ of Ring E is independently as described above.

In certain embodiments, the present invention provides a compound wherein Ring A is a 5 membered saturated or partially unsaturated ring having 1-2 heteroatoms independently selected from nitrogen, oxygen, or sulfur, and wherein Ring E is a 7 membered saturated or partially unsaturated carbocycle, wherein each R⁴ and R⁵ of Ring E is independently as described above.

In some embodiments, the present invention provides a compound wherein Ring A is a 6 membered saturated or partially unsaturated carbocycle, and wherein Ring E is a 4-7 membered saturated, partially unsaturated, or aromatic ring having 0-2 heteroatoms independently selected from nitrogen, oxygen, or sulfur, and wherein each R⁴ of Ring E is independently selected from halogen, CN, R, OR, a suitably protected hydroxyl group, SR, a suitably protected thiol group, S(O)R, SO₂R, OSO₂R, N(R)₂, a suitably protected amino group, N(R)C(O)R, N(R)C(O)C(O)R, N(R)C(O)N(R)₂, N(R)C(O)OR, C(O)OR, OC(O)R, C(O)N(R)₂, or OC(O)N(R)₂, or:

two R⁴ on the same carbon are optionally taken together to form an optionally substituted 3-8 membered saturated or partially unsaturated spirofused ring having 0-4 heteroatoms independently selected from nitrogen, oxygen, or sulfur, or:

two R⁴ on the same carbon are optionally taken together to form an oxo moiety, an oxime, an optionally substituted hydrazone, or an optionally substituted imine;

and wherein each R⁵ of Ring E is independently T-C(R′)₃, T-C(R′)₂C(R″)₃, OR, a suitably protected hydroxyl group, SR, a suitably protected thiol group, S(O)R, SO₂R, OSO₂R, N(R)₂, a suitably protected amino group, N(R)C(O)R, N(R)C(O)C(O)R, N(R)C(O)N(R)₂, N(R)C(O)OR, C(O)OR, OC(O)R, C(O)N(R)₂, or OC(O)N(R)₂, an optionally substituted 3-8 membered saturated, partially unsaturated, or aryl monocyclic ring having 0-4 heteroatoms independently selected from nitrogen, oxygen, or sulfur, an optionally substituted 8-10 membered saturated, partially unsaturated, or aryl bicyclic ring having 0-4 heteroatoms independently selected from nitrogen, oxygen, or sulfur, or:

two R⁵ on the same carbon are optionally taken together to form an oxo moiety, an oxime, an optionally substituted hydrazone, an optionally substituted imine, or an optionally substituted 3-8 membered saturated or partially unsaturated spirocycle having 0-4 heteroatoms independently selected from nitrogen, oxygen, or sulfur;

each T is independently a valence bond or an optionally substituted straight or branched, saturated or unsaturated, C₁₋₆ alkylene chain wherein up to two methylene units of T are optionally and independently replaced by —O—, —N(R)—, —S—, —C(O)—, —S(O)—, or —S(O)₂—;

each R′ and R″ is independently selected from halogen, R, OR, SR, S(O)R, SO₂R, OSO₂R, N(R)₂, N(R)C(O)R, N(R)C(O)C(O)R, N(R)C(O)N(R)₂, N(R)C(O)OR, N(R)S(O)R, N(R)SO₂R, N(R)SO₂OR C(O)OR, OC(O)R, C(O)N(R)₂, OC(O)N(R)₂, or an optionally substituted 3-8 membered saturated, partially unsaturated, or aryl monocyclic ring having 0-4 heteroatoms independently selected from nitrogen, oxygen, or sulfur, or an optionally substituted 8-10 membered saturated, partially unsaturated, or aryl bicyclic ring having 0-4 heteroatoms independently selected from nitrogen, oxygen, or sulfur, or:

two R′ are optionally taken together to form an oxo moiety, an oxime, an optionally substituted hydrazone, an optionally substituted imine, or an optionally substituted 3-8 membered saturated or partially unsaturated ring having 0-4 heteroatoms independently selected from nitrogen, oxygen, or sulfur, or:

two R″ are optionally taken together to form an oxo moiety, an oxime, an optionally substituted hydrazone, an optionally substituted imine, or an optionally substituted 3-8 membered saturated or partially unsaturated ring having 0-4 heteroatoms independently selected from nitrogen, oxygen, or sulfur; or

R⁶ and R⁵ are optionally taken together to form an optionally substituted 3-8 membered saturated, partially unsaturated, or aryl ring having 0-4 heteroatoms independently selected from nitrogen, oxygen, or sulfur.

In certain embodiments, the present invention provides a compound wherein Ring A is a 6 membered saturated or partially unsaturated carbocycle, and wherein Ring E is a 4 membered saturated ring having 1-2 heteroatoms independently selected from nitrogen, oxygen, or sulfur, and wherein each R⁴ and R⁵ of Ring E is independently as described above.

In certain embodiments, the present invention provides a compound wherein Ring A is a 6 membered saturated or partially unsaturated carbocycle, and wherein Ring E is a 4 membered saturated carbocycle, wherein each R⁴ and R⁵ of Ring E is independently as described above.

In certain embodiments, the present invention provides a compound wherein Ring A is a 6 membered saturated or partially unsaturated carbocycle, and wherein Ring E is a 5 membered saturated, partially unsaturated, or aromatic ring having 1-2 heteroatoms independently selected from nitrogen, oxygen, or sulfur, and wherein each R⁴ and R⁵ of Ring E is independently as described above.

In certain embodiments, the present invention provides a compound wherein Ring A is a 6 membered saturated or partially unsaturated carbocycle, and wherein Ring E is a 5 membered saturated or partially unsaturated carbocycle, wherein each R⁴ and R⁵ of Ring E is independently as described above.

In certain embodiments, the present invention provides a compound wherein Ring A is a 6 membered saturated or partially unsaturated carbocycle, and wherein Ring E is a 6 membered saturated, partially unsaturated, or aromatic ring having 1-2 heteroatoms independently selected from nitrogen, oxygen, or sulfur, and wherein each R⁴ and R⁵ of Ring E is independently as described above.

In certain embodiments, the present invention provides a compound wherein Ring A is a 6 membered saturated or partially unsaturated carbocycle, and wherein Ring E is a 6 membered saturated, partially unsaturated, or aromatic carbocycle, wherein each R⁴ and R⁵ of Ring E is independently as described above.

In certain embodiments, the present invention provides a compound wherein Ring A is a 6 membered saturated or partially unsaturated carbocycle, and wherein Ring E is a 7 membered saturated or partially unsaturated ring having 1-2 heteroatoms independently selected from nitrogen, oxygen, or sulfur, and wherein each R⁴ and R⁵ of Ring E is independently as described above.

In certain embodiments, the present invention provides a compound wherein Ring A is a 6 membered saturated or partially unsaturated carbocycle, and wherein Ring E is a 7 membered saturated or partially unsaturated carbocycle, wherein each R⁴ and R⁵ of Ring E is independently as described above.

In some embodiments, the present invention provides a compound wherein Ring A is a 6 membered saturated or partially unsaturated ring having 1-2 heteroatoms independently selected from nitrogen, oxygen, or sulfur, and wherein Ring E is a 4-7 membered saturated, partially unsaturated, or aromatic ring having 0-2 heteroatoms independently selected from nitrogen, oxygen, or sulfur, and wherein Ring E is a 4-7 membered saturated, partially unsaturated, or aromatic ring having 0-2 heteroatoms independently selected from nitrogen, oxygen, or sulfur, and wherein each R⁴ of Ring E is independently selected from halogen, CN, R, OR, a suitably protected hydroxyl group, SR, a suitably protected thiol group, S(O)R, SO₂R, OSO₂R, N(R)₂, a suitably protected amino group, N(R)C(O)R, N(R)C(O)C(O)R, N(R)C(O)N(R)₂, N(R)C(O)OR, C(O)OR, OC(O)R, C(O)N(R)₂, or OC(O)N(R)₂, or:

two R⁴ on the same carbon are optionally taken together to form an optionally substituted 3-8 membered saturated or partially unsaturated spirofused ring having 0-4 heteroatoms independently selected from nitrogen, oxygen, or sulfur, or:

two R⁴ on the same carbon are optionally taken together to form an oxo moiety, an oxime, an optionally substituted hydrazone, or an optionally substituted imine;

and wherein each R⁵ of Ring E is independently T-C(R′)₃, T-C(R′)₂C(R″)₃, OR, a suitably protected hydroxyl group, SR, a suitably protected thiol group, S(O)R, SO₂R, OSO₂R, N(R)₂, a suitably protected amino group, N(R)C(O)R, N(R)C(O)C(O)R, N(R)C(O)N(R)₂, N(R)C(O)OR, C(O)OR, OC(O)R, C(O)N(R)₂, or OC(O)N(R)₂, an optionally substituted 3-8 membered saturated, partially unsaturated, or aryl monocyclic ring having 0-4 heteroatoms independently selected from nitrogen, oxygen, or sulfur, an optionally substituted 8-10 membered saturated, partially unsaturated, or aryl bicyclic ring having 0-4 heteroatoms independently selected from nitrogen, oxygen, or sulfur, or:

two R⁵ on the same carbon are optionally taken together to form an oxo moiety, an oxime, an optionally substituted hydrazone, an optionally substituted imine, or an optionally substituted 3-8 membered saturated or partially unsaturated spirocycle having 0-4 heteroatoms independently selected from nitrogen, oxygen, or sulfur;

each T is independently a valence bond or an optionally substituted straight or branched, saturated or unsaturated, C₁₋₆ alkylene chain wherein up to two methylene units of T are optionally and independently replaced by —O—, —N(R)—, —S—, —C(O)—, —S(O)—, or —S(O)₂—;

each R′ and R″ is independently selected from halogen, R, OR, SR, S(O)R, SO₂R, OSO₂R, N(R)₂, N(R)C(O)R, N(R)C(O)C(O)R, N(R)C(O)N(R)₂, N(R)C(O)OR, N(R)S(O)R, N(R)SO₂R, N(R)SO₂OR C(O)OR, OC(O)R, C(O)N(R)₂, OC(O)N(R)₂, or an optionally substituted 3-8 membered saturated, partially unsaturated, or aryl monocyclic ring having 0-4 heteroatoms independently selected from nitrogen, oxygen, or sulfur, or an optionally substituted 8-10 membered saturated, partially unsaturated, or aryl bicyclic ring having 0-4 heteroatoms independently selected from nitrogen, oxygen, or sulfur, or:

two R′ are optionally taken together to form an oxo moiety, an oxime, an optionally substituted hydrazone, an optionally substituted imine, or an optionally substituted 3-8 membered saturated or partially unsaturated ring having 0-4 heteroatoms independently selected from nitrogen, oxygen, or sulfur, or:

two R″ are optionally taken together to form an oxo moiety, an oxime, an optionally substituted hydrazone, an optionally substituted imine, or an optionally substituted 3-8 membered saturated or partially unsaturated ring having 0-4 heteroatoms independently selected from nitrogen, oxygen, or sulfur; or

R⁶ and R⁵ are optionally taken together to form an optionally substituted 3-8 membered saturated, partially unsaturated, or aryl ring having 0-4 heteroatoms independently selected from nitrogen, oxygen, or sulfur.

In certain embodiments, the present invention provides a compound wherein Ring A is a 6 membered saturated or partially unsaturated ring having 1-2 heteroatoms independently selected from nitrogen, oxygen, or sulfur, and wherein Ring E is a 4 membered saturated ring having 1-2 heteroatoms independently selected from nitrogen, oxygen, or sulfur, and wherein each R⁴ and R⁵ of Ring E is independently as described above.

In certain embodiments, the present invention provides a compound wherein Ring A is a 6 membered saturated or partially unsaturated ring having 1-2 heteroatoms independently selected from nitrogen, oxygen, or sulfur, and wherein Ring E is a 4 membered saturated carbocycle, wherein each R⁴ and R⁵ of Ring E is independently as described above.

In certain embodiments, the present invention provides a compound wherein Ring A is a 6 membered saturated or partially unsaturated ring having 1-2 heteroatoms independently selected from nitrogen, oxygen, or sulfur, and wherein Ring E is a 5 membered saturated, partially unsaturated, or aromatic ring having 1-2 heteroatoms independently selected from nitrogen, oxygen, or sulfur, and wherein each R⁴ and R⁵ of Ring E is independently as described above.

In certain embodiments, the present invention provides a compound wherein Ring A is a 6 membered saturated or partially unsaturated ring having 1-2 heteroatoms independently selected from nitrogen, oxygen, or sulfur, and wherein Ring E is a 5 membered saturated or partially unsaturated carbocycle, wherein each R⁴ and R⁵ of Ring E is independently as described above.

In certain embodiments, the present invention provides a compound wherein Ring A is a 6 membered saturated or partially unsaturated ring having 1-2 heteroatoms independently selected from nitrogen, oxygen, or sulfur, and wherein Ring E is a 6 membered saturated, partially unsaturated, or aromatic ring having 1-2 heteroatoms independently selected from nitrogen, oxygen, or sulfur, and wherein each R⁴ and R⁵ of Ring E is independently as described above.

In certain embodiments, the present invention provides a compound wherein Ring A is a 6 membered saturated or partially unsaturated ring having 1-2 heteroatoms independently selected from nitrogen, oxygen, or sulfur, and wherein Ring E is a 6 membered saturated, partially unsaturated, or aromatic carbocycle, wherein each R⁴ and R⁵ of Ring E is independently as described above.

In certain embodiments, the present invention provides a compound wherein Ring A is a 6 membered saturated or partially unsaturated ring having 1-2 heteroatoms independently selected from nitrogen, oxygen, or sulfur, and wherein Ring E is a 7 membered saturated or partially unsaturated ring having 1-2 heteroatoms independently selected from nitrogen, oxygen, or sulfur, and wherein each R⁴ and R⁵ of Ring E is independently as described above.

In certain embodiments, the present invention provides a compound wherein Ring A is a 6 membered saturated or partially unsaturated ring having 1-2 heteroatoms independently selected from nitrogen, oxygen, or sulfur, and wherein Ring E is a 7 membered saturated or partially unsaturated carbocycle, wherein each R⁴ and R⁵ of Ring E is independently as described above.

In some embodiments, the present invention provides a compound wherein Ring A is a 7 membered saturated or partially unsaturated carbocycle, and wherein Ring E is a 4-7 membered saturated, partially unsaturated, or aromatic ring having 0-2 heteroatoms independently selected from nitrogen, oxygen, or sulfur, and wherein each R⁴ of Ring E is independently selected from halogen, CN, R, OR, a suitably protected hydroxyl group, SR, a suitably protected thiol group, S(O)R, SO₂R, OSO₂R, N(R)₂, a suitably protected amino group, N(R)C(O)R, N(R)C(O)C(O)R, N(R)C(O)N(R)₂, N(R)C(O)OR, C(O)OR, OC(O)R, C(O)N(R)₂, or OC(O)N(R)₂, or:

two R⁴ on the same carbon are optionally taken together to form an optionally substituted 3-8 membered saturated or partially unsaturated spirofused ring having 0-4 heteroatoms independently selected from nitrogen, oxygen, or sulfur, or:

two R⁴ on the same carbon are optionally taken together to form an oxo moiety, an oxime, an optionally substituted hydrazone, or an optionally substituted imine;

and wherein each R⁵ of Ring E is independently T-C(R′)₃, T-C(R′)₂C(R″)₃, OR, a suitably protected hydroxyl group, SR, a suitably protected thiol group, S(O)R, SO₂R, OSO₂R, N(R)₂, a suitably protected amino group, N(R)C(O)R, N(R)C(O)C(O)R, N(R)C(O)N(R)₂, N(R)C(O)OR, C(O)OR, OC(O)R, C(O)N(R)₂, or OC(O)N(R)₂, an optionally substituted 3-8 membered saturated, partially unsaturated, or aryl monocyclic ring having 0-4 heteroatoms independently selected from nitrogen, oxygen, or sulfur, an optionally substituted 8-10 membered saturated, partially unsaturated, or aryl bicyclic ring having 0-4 heteroatoms independently selected from nitrogen, oxygen, or sulfur, or:

two R⁵ on the same carbon are optionally taken together to form an oxo moiety, an oxime, an optionally substituted hydrazone, an optionally substituted imine, or an optionally substituted 3-8 membered saturated or partially unsaturated spirocycle having 0-4 heteroatoms independently selected from nitrogen, oxygen, or sulfur;

each T is independently a valence bond or an optionally substituted straight or branched, saturated or unsaturated, C₁₋₆ alkylene chain wherein up to two methylene units of T are optionally and independently replaced by —O—, —N(R)—, —S—, —C(O)—, —S(O)—, or —S(O)₂—;

each R′ and R″ is independently selected from halogen, R, OR, SR, S(O)R, SO₂R, OSO₂R, N(R)₂, N(R)C(O)R, N(R)C(O)C(O)R, N(R)C(O)N(R)₂, N(R)C(O)OR, N(R)S(O)R, N(R)SO₂R, N(R)SO₂OR C(O)OR, OC(O)R, C(O)N(R)₂, OC(O)N(R)₂, or an optionally substituted 3-8 membered saturated, partially unsaturated, or aryl monocyclic ring having 0-4 heteroatoms independently selected from nitrogen, oxygen, or sulfur, or an optionally substituted 8-10 membered saturated, partially unsaturated, or aryl bicyclic ring having 0-4 heteroatoms independently selected from nitrogen, oxygen, or sulfur, or:

two R′ are optionally taken together to form an oxo moiety, an oxime, an optionally substituted hydrazone, an optionally substituted imine, or an optionally substituted 3-8 membered saturated or partially unsaturated ring having 0-4 heteroatoms independently selected from nitrogen, oxygen, or sulfur, or:

two R″ are optionally taken together to form an oxo moiety, an oxime, an optionally substituted hydrazone, an optionally substituted imine, or an optionally substituted 3-8 membered saturated or partially unsaturated ring having 0-4 heteroatoms independently selected from nitrogen, oxygen, or sulfur; or

R⁶ and R⁵ are optionally taken together to form an optionally substituted 3-8 membered saturated, partially unsaturated, or aryl ring having 0-4 heteroatoms independently selected from nitrogen, oxygen, or sulfur.

In certain embodiments, the present invention provides a compound wherein Ring A is a 7 membered saturated or partially unsaturated carbocycle, and wherein Ring E is a 4 membered saturated ring having 1-2 heteroatoms independently selected from nitrogen, oxygen, or sulfur, and wherein each R⁴ and R⁵ of Ring E is independently as described above.

In certain embodiments, the present invention provides a compound wherein Ring A is a 7 membered saturated or partially unsaturated carbocycle, and wherein Ring E is a 4 membered saturated carbocycle, wherein each R⁴ and R⁵ of Ring E is independently as described above.

In certain embodiments, the present invention provides a compound wherein Ring A is a 7 membered saturated or partially unsaturated carbocycle, and wherein Ring E is a 5 membered saturated, partially unsaturated, or aromatic ring having 1-2 heteroatoms independently selected from nitrogen, oxygen, or sulfur, and wherein each R⁴ and R⁵ of Ring E is independently as described above.

In certain embodiments, the present invention provides a compound wherein Ring A is a 7 membered saturated or partially unsaturated carbocycle, and wherein Ring E is a 5 membered saturated or partially unsaturated carbocycle, wherein each R⁴ and R⁵ of Ring E is independently as described above.

In certain embodiments, the present invention provides a compound wherein Ring A is a 7 membered saturated or partially unsaturated carbocycle, and wherein Ring E is a 6 membered saturated, partially unsaturated, or aromatic ring having 1-2 heteroatoms independently selected from nitrogen, oxygen, or sulfur, and wherein each R⁴ and R⁵ of Ring E is independently as described above.

In certain embodiments, the present invention provides a compound wherein Ring A is a 7 membered saturated or partially unsaturated carbocycle, and wherein Ring E is a 6 membered saturated, partially unsaturated, or aromatic carbocycle, wherein each R⁴ and R⁵ of Ring E is independently as described above.

In certain embodiments, the present invention provides a compound wherein Ring A is a 7 membered saturated or partially unsaturated carbocycle, and wherein Ring E is a 7 membered saturated or partially unsaturated ring having 1-2 heteroatoms independently selected from nitrogen, oxygen, or sulfur, and wherein each R⁴ and R⁵ of Ring E is independently as described above.

In certain embodiments, the present invention provides a compound wherein Ring A is a 7 membered saturated or partially unsaturated carbocycle, and wherein Ring E is a 7 membered saturated or partially unsaturated carbocycle, wherein each R⁴ and R⁵ of Ring E is independently as described above.

In some embodiments, the present invention provides a compound wherein Ring A is a 7 membered saturated or partially unsaturated ring having 1-2 heteroatoms independently selected from nitrogen, oxygen, or sulfur, and wherein Ring E is a 4-7 membered saturated, partially unsaturated, or aromatic ring having 0-2 heteroatoms independently selected from nitrogen, oxygen, or sulfur, and wherein each R⁴ of Ring E is independently selected from halogen, CN, R, OR, a suitably protected hydroxyl group, SR, a suitably protected thiol group, S(O)R, SO₂R, OSO₂R, N(R)₂, a suitably protected amino group, N(R)C(O)R, N(R)C(O)C(O)R, N(R)C(O)N(R)₂, N(R)C(O)OR, C(O)OR, OC(O)R, C(O)N(R)₂, or OC(O)N(R)₂, or:

two R⁴ on the same carbon are optionally taken together to form an optionally substituted 3-8 membered saturated or partially unsaturated spirofused ring having 0-4 heteroatoms independently selected from nitrogen, oxygen, or sulfur, or:

two R⁴ on the same carbon are optionally taken together to form an oxo moiety, an oxime, an optionally substituted hydrazone, or an optionally substituted imine;

and wherein each R⁵ of Ring E is independently T-C(R′)₃, T-C(R′)₂C(R″)₃, OR, a suitably protected hydroxyl group, SR, a suitably protected thiol group, S(O)R, SO₂R, OSO₂R, N(R)₂, a suitably protected amino group, N(R)C(O)R, N(R)C(O)C(O)R, N(R)C(O)N(R)₂, N(R)C(O)OR, C(O)OR, OC(O)R, C(O)N(R)₂, or OC(O)N(R)₂, an optionally substituted 3-8 membered saturated, partially unsaturated, or aryl monocyclic ring having 0-4 heteroatoms independently selected from nitrogen, oxygen, or sulfur, an optionally substituted 8-10 membered saturated, partially unsaturated, or aryl bicyclic ring having 0-4 heteroatoms independently selected from nitrogen, oxygen, or sulfur, or:

two R⁵ on the same carbon are optionally taken together to form an oxo moiety, an oxime, an optionally substituted hydrazone, an optionally substituted imine, or an optionally substituted 3-8 membered saturated or partially unsaturated spirocycle having 0-4 heteroatoms independently selected from nitrogen, oxygen, or sulfur;

each T is independently a valence bond or an optionally substituted straight or branched, saturated or unsaturated, C₁₋₆ alkylene chain wherein up to two methylene units of T are optionally and independently replaced by —O—, —N(R)—, —S—, —C(O)—, —S(O)—, or —S(O)₂—;

each R′ and R″ is independently selected from halogen, R, OR, SR, S(O)R, SO₂R, OSO₂R, N(R)₂, N(R)C(O)R, N(R)C(O)C(O)R, N(R)C(O)N(R)₂, N(R)C(O)OR, N(R)S(O)R, N(R)SO₂R, N(R)SO₂OR C(O)OR, OC(O)R, C(O)N(R)₂, OC(O)N(R)₂, or an optionally substituted 3-8 membered saturated, partially unsaturated, or aryl monocyclic ring having 0-4 heteroatoms independently selected from nitrogen, oxygen, or sulfur, or an optionally substituted 8-10 membered saturated, partially unsaturated, or aryl bicyclic ring having 0-4 heteroatoms independently selected from nitrogen, oxygen, or sulfur, or:

two R′ are optionally taken together to form an oxo moiety, an oxime, an optionally substituted hydrazone, an optionally substituted imine, or an optionally substituted 3-8 membered saturated or partially unsaturated ring having 0-4 heteroatoms independently selected from nitrogen, oxygen, or sulfur, or:

two R″ are optionally taken together to form an oxo moiety, an oxime, an optionally substituted hydrazone, an optionally substituted imine, or an optionally substituted 3-8 membered saturated or partially unsaturated ring having 0-4 heteroatoms independently selected from nitrogen, oxygen, or sulfur; or

R⁶ and R⁵ are optionally taken together to form an optionally substituted 3-8 membered saturated, partially unsaturated, or aryl ring having 0-4 heteroatoms independently selected from nitrogen, oxygen, or sulfur.

In certain embodiments, the present invention provides a compound wherein Ring A is a 7 membered saturated or partially unsaturated ring having 1-2 heteroatoms independently selected from nitrogen, oxygen, or sulfur, and wherein Ring E is a 4 membered saturated ring having 1-2 heteroatoms independently selected from nitrogen, oxygen, or sulfur, and wherein each R⁴ and R⁵ of Ring E is independently as described above.

In certain embodiments, the present invention provides a compound wherein Ring A is a 7 membered saturated or partially unsaturated ring having 1-2 heteroatoms independently selected from nitrogen, oxygen, or sulfur, and wherein Ring E is a 4 membered saturated carbocycle, wherein each R⁴ and R⁵ of Ring E is independently as described above.

In certain embodiments, the present invention provides a compound wherein Ring A is a 7 membered saturated or partially unsaturated ring having 1-2 heteroatoms independently selected from nitrogen, oxygen, or sulfur, and wherein Ring E is a 5 membered saturated, partially unsaturated, or aromatic ring having 1-2 heteroatoms independently selected from nitrogen, oxygen, or sulfur, and wherein each R⁴ and R⁵ of Ring E is independently as described above.

In certain embodiments, the present invention provides a compound wherein Ring A is a 7 membered saturated or partially unsaturated ring having 1-2 heteroatoms independently selected from nitrogen, oxygen, or sulfur, and wherein Ring E is a 5 membered saturated or partially unsaturated carbocycle, wherein each R⁴ and R⁵ of Ring E is independently as described above.

In certain embodiments, the present invention provides a compound wherein Ring A is a 7 membered saturated or partially unsaturated ring having 1-2 heteroatoms independently selected from nitrogen, oxygen, or sulfur, and wherein Ring E is a 6 membered saturated, partially unsaturated, or aromatic ring having 1-2 heteroatoms independently selected from nitrogen, oxygen, or sulfur, and wherein each R⁴ and R⁵ of Ring E is independently as described above.

In certain embodiments, the present invention provides a compound wherein Ring A is a 7 membered saturated or partially unsaturated ring having 1-2 heteroatoms independently selected from nitrogen, oxygen, or sulfur, and wherein Ring E is a 6 membered saturated, partially unsaturated, or aromatic carbocycle, wherein each R⁴ and R⁵ of Ring E is independently as described above.

In certain embodiments, the present invention provides a compound wherein Ring A is a 7 membered saturated or partially unsaturated ring having 1-2 heteroatoms independently selected from nitrogen, oxygen, or sulfur, and wherein Ring E is a 7 membered saturated or partially unsaturated ring having 1-2 heteroatoms independently selected from nitrogen, oxygen, or sulfur, and wherein each R⁴ and R⁵ of Ring E is independently as described above.

In certain embodiments, the present invention provides a compound wherein Ring A is a 7 membered saturated or partially unsaturated ring having 1-2 heteroatoms independently selected from nitrogen, oxygen, or sulfur, and wherein Ring E is a 7 membered saturated or partially unsaturated carbocycle, wherein each R⁴ and R⁵ of Ring E is independently as described above.

### Exemplary Ring D/Ring E Combinations

In some embodiments, the present invention provides a compound wherein Ring E is a 4 membered saturated carbocycle, wherein R³ and R⁸ of Ring D are each independently selected from halogen, CN, R, OR, a suitably protected hydroxyl group, SR, a suitably protected thiol group, S(O)R, SO₂R, OSO₂R, N(R)₂, a suitably protected amino group, N(R)C(O)R, N(R)C(O)C(O)R, N(R)C(O)N(R)₂, N(R)C(O)OR, C(O)OR, OC(O)R, C(O)N(R)₂, or OC(O)N(R)₂, and wherein R⁷ and R^{7′} of Ring D are each independently selected from halogen, CN, N₃, R, OR, a suitably protected hydroxyl group, SR, a suitably protected thiol group, SO₂R, OSO₂R, N(R)₂, a suitably protected amino group, N(R)C(O)R, N(R)C(O)C(O)R, N(R)C(O)N(R)₂, N(R)C(O)OR, C(O)OR, OC(O)R, C(O)N(R)₂, or OC(O)N(R)₂, or R⁷ and R^{7′} are taken together to form an oxo moiety.

In certain embodiments, the present invention provides a compound wherein Ring E is a 4 membered saturated carbocycle, wherein R³ and R⁸ of Ring D are each independently selected from halogen, R, OR, or a suitably protected hydroxyl group, and wherein R⁷ and R^{7′} of Ring D are each independently selected from halogen, R, OR, a suitably protected hydroxyl group, or R⁷ and R^{7′} are taken together to form an oxo moiety.

In some embodiments, the present invention provides a compound wherein Ring E is a 4 membered saturated ring having 1-2 heteroatoms independently selected from nitrogen, oxygen, or sulfur, and wherein R³ and R⁸ of Ring D are each independently selected from halogen, CN, R, OR, a suitably protected hydroxyl group, SR, a suitably protected thiol group, S(O)R, SO₂R, OSO₂R, N(R)₂, a suitably protected amino group, N(R)C(O)R, N(R)C(O)C(O)R, N(R)C(O)N(R)₂, N(R)C(O)OR, C(O)OR, OC(O)R, C(O)N(R)₂, or OC(O)N(R)₂, and wherein R⁷ and R^{7′} of Ring D are each independently selected from halogen, CN, N₃, R, OR, a suitably protected hydroxyl group, SR, a suitably protected thiol group, SO₂R, OSO₂R, N(R)₂, a suitably protected amino group, N(R)C(O)R, N(R)C(O)C(O)R, N(R)C(O)N(R)₂, N(R)C(O)OR, C(O)OR, OC(O)R, C(O)N(R)₂, or OC(O)N(R)₂, or R⁷ and R^{7′} are taken together to form an oxo moiety.

In certain embodiments, the present invention provides a compound wherein Ring E is a 4 membered saturated ring having 1-2 heteroatoms independently selected from nitrogen, oxygen, or sulfur, and wherein R³ and R⁸ of Ring D are each independently selected from halogen, R, OR, or a suitably protected hydroxyl group, and wherein R⁷ and R^{7′} of Ring D are each independently selected from halogen, R, OR, a suitably protected hydroxyl group, or R⁷ and R^{7′} are taken together to form an oxo moiety.

In some embodiments, the present invention provides a compound wherein Ring E is a 5 membered saturated or partially unsaturated carbocycle, wherein R³ and R⁸ of Ring D are each independently selected from halogen, CN, R, OR, a suitably protected hydroxyl group, SR, a suitably protected thiol group, S(O)R, SO₂R, OSO₂R, N(R)₂, a suitably protected amino group, N(R)C(O)R, N(R)C(O)C(O)R, N(R)C(O)N(R)₂, N(R)C(O)OR, C(O)OR, OC(O)R, C(O)N(R)₂, or OC(O)N(R)₂, and wherein R⁷ and R^{7′} of Ring D are each independently selected from halogen, CN, N₃, R, OR, a suitably protected hydroxyl group, SR, a suitably protected thiol group, SO₂R, OSO₂R, N(R)₂, a suitably protected amino group, N(R)C(O)R, N(R)C(O)C(O)R, N(R)C(O)N(R)₂, N(R)C(O)OR, C(O)OR, OC(O)R, C(O)N(R)₂, or OC(O)N(R)₂, or R⁷ and R^{7′} are taken together to form an oxo moiety

In certain embodiments, the present invention provides a compound wherein Ring E is a 5 membered saturated carbocycle, wherein R³ and R⁸ of Ring D are each independently selected from halogen, R, OR, or a suitably protected hydroxyl group, and wherein R⁷ and R^{7′} of Ring D are each independently selected from halogen, R, OR, a suitably protected hydroxyl group, or R⁷ and R^{7′} are taken together to form an oxo moiety.

In certain embodiments, the present invention provides a compound wherein Ring E is a 5 membered partially unsaturated carbocycle, wherein R³ and R⁸ of Ring D are each independently selected from halogen, R, OR, or a suitably protected hydroxyl group, and wherein R⁷ and R^{7′} of Ring D are each independently selected from halogen, R, OR, a suitably protected hydroxyl group, or R⁷ and R^{7′} are taken together to form an oxo moiety.

In some embodiments, the present invention provides a compound wherein Ring E is a 5 membered saturated, partially unsaturated, or aromatic ring having 1-2 heteroatoms independently selected from nitrogen, oxygen, or sulfur, and wherein R³ and R⁸ of Ring D are each independently selected from halogen, CN, R, OR, a suitably protected hydroxyl group, SR, a suitably protected thiol group, S(O)R, SO₂R, OSO₂R, N(R)₂, a suitably protected amino group, N(R)C(O)R, N(R)C(O)C(O)R, N(R)C(O)N(R)₂, N(R)C(O)OR, C(O)OR, OC(O)R, C(O)N(R)₂, or OC(O)N(R)₂, and wherein R⁷ and R^{7′} of Ring D are each independently selected from halogen, CN, N₃, R, OR, a suitably protected hydroxyl group, SR, a suitably protected thiol group, SO₂R, OSO₂R, N(R)₂, a suitably protected amino group, N(R)C(O)R, N(R)C(O)C(O)R, N(R)C(O)N(R)₂, N(R)C(O)OR, C(O)OR, OC(O)R, C(O)N(R)₂, or OC(O)N(R)₂, or R⁷ and R^{7′} are taken together to form an oxo moiety.

In certain embodiments, the present invention provides a compound wherein Ring E is a 5 membered saturated ring having 1-2 heteroatoms independently selected from nitrogen, oxygen, or sulfur, and wherein R³ and R⁸ of Ring D are each independently selected from halogen, R, OR, or a suitably protected hydroxyl group, and wherein R⁷ and R^{7′} of Ring D are each independently selected from halogen, R, OR, a suitably protected hydroxyl group, or R⁷ and R^{7′} are taken together to form an oxo moiety.

In certain embodiments, the present invention provides a compound wherein Ring E is a 5 membered partially unsaturated ring having 1-2 heteroatoms independently selected from nitrogen, oxygen, or sulfur, and wherein R³ and R⁸ of Ring D are each independently selected from halogen, R, OR, or a suitably protected hydroxyl group, and wherein R⁷ and R^{7′} of Ring D are each independently selected from halogen, R, OR, a suitably protected hydroxyl group, or R⁷ and R^{7′} are taken together to form an oxo moiety.

In certain embodiments, the present invention provides a compound wherein Ring E is a 5 membered aromatic ring having 1-2 heteroatoms independently selected from nitrogen, oxygen, or sulfur, and wherein R³ and R⁸ of Ring D are each independently selected from halogen, R, OR, or a suitably protected hydroxyl group, and wherein R⁷ and R^{7′} of Ring D are each independently selected from halogen, R, OR, a suitably protected hydroxyl group, or R⁷ and R^{7′} are taken together to form an oxo moiety.

In some embodiments, the present invention provides a compound wherein Ring E is a 6 membered saturated, partially unsaturated, or aromatic carbocycle, and wherein R³ and R⁸ of Ring D are each independently selected from halogen, CN, R, OR, a suitably protected hydroxyl group, SR, a suitably protected thiol group, S(O)R, SO₂R, OSO₂R, N(R)₂, a suitably protected amino group, N(R)C(O)R, N(R)C(O)C(O)R, N(R)C(O)N(R)₂, N(R)C(O)OR, C(O)OR, OC(O)R, C(O)N(R)₂, or OC(O)N(R)₂, and wherein R⁷ and R^{7′} of Ring D are each independently selected from halogen, CN, N₃, R, OR, a suitably protected hydroxyl group, SR, a suitably protected thiol group, SO₂R, OSO₂R, N(R)₂, a suitably protected amino group, N(R)C(O)R, N(R)C(O)C(O)R, N(R)C(O)N(R)₂, N(R)C(O)OR, C(O)OR, OC(O)R, C(O)N(R)₂, or OC(O)N(R)₂, or R⁷ and R^{7′} are taken together to form an oxo moiety.

In certain embodiments, the present invention provides a compound wherein Ring E is a 6 membered saturated carbocycle, wherein R³ and R⁸ of Ring D are each independently selected from halogen, R, OR, or a suitably protected hydroxyl group, and wherein R⁷ and R^{7′} of Ring D are each independently selected from halogen, R, OR, a suitably protected hydroxyl group, or R⁷ and R^{7′} are taken together to form an oxo moiety.

In certain embodiments, the present invention provides a compound wherein Ring E is a 6 membered partially unsaturated carbocycle, wherein R³ and R⁸ of Ring D are each independently selected from halogen, R, OR, or a suitably protected hydroxyl group, and wherein R⁷ and R^{7′} of Ring D are each independently selected from halogen, R, OR, a suitably protected hydroxyl group, or R⁷ and R^{7′} are taken together to form an oxo moiety.

In certain embodiments, the present invention provides a compound wherein Ring E is a 6 membered aromatic carbocycle, wherein R³ and R⁸ of Ring D are each independently selected from halogen, R, OR, or a suitably protected hydroxyl group, and wherein R⁷ and R^{7′} of Ring D are each independently selected from halogen, R, OR, a suitably protected hydroxyl group, or R⁷ and R^{7′} are taken together to form an oxo moiety.

In some embodiments, the present invention provides a compound wherein Ring E is a 6 membered saturated, partially unsaturated, or aromatic ring having 1-2 heteroatoms independently selected from nitrogen, oxygen, or sulfur, and wherein R³ and R⁸ of Ring D are each independently selected from halogen, CN, R, OR, a suitably protected hydroxyl group, SR, a suitably protected thiol group, S(O)R, SO₂R, OSO₂R, N(R)₂, a suitably protected amino group, N(R)C(O)R, N(R)C(O)C(O)R, N(R)C(O)N(R)₂, N(R)C(O)OR, C(O)OR, OC(O)R, C(O)N(R)₂, or OC(O)N(R)₂, and wherein R⁷ and R^{7′} of Ring D are each independently selected from halogen, CN, N₃, R, OR, a suitably protected hydroxyl group, SR, a suitably protected thiol group, SO₂R, OSO₂R, N(R)₂, a suitably protected amino group, N(R)C(O)R, N(R)C(O)C(O)R, N(R)C(O)N(R)₂, N(R)C(O)OR, C(O)OR, OC(O)R, C(O)N(R)₂, or OC(O)N(R)₂, or R⁷ and R^{7′} are taken together to form an oxo moiety.

In certain embodiments, the present invention provides a compound wherein Ring E is a 6 membered saturated ring having 1-2 heteroatoms independently selected from nitrogen, oxygen, or sulfur, and wherein R³ and R⁸ of Ring D are each independently selected from halogen, R, OR, or a suitably protected hydroxyl group, and wherein R⁷ and R^{7′} of Ring D are each independently selected from halogen, R, OR, a suitably protected hydroxyl group, or R⁷ and R^{7′} are taken together to form an oxo moiety.

In certain embodiments, the present invention provides a compound wherein Ring E is a 6 membered partially unsaturated ring having 1-2 heteroatoms independently selected from nitrogen, oxygen, or sulfur, and wherein R³ and R⁸ of Ring D are each independently selected from halogen, R, OR, or a suitably protected hydroxyl group, and wherein R⁷ and R^{7′} of Ring D are each independently selected from halogen, R, OR, a suitably protected hydroxyl group, or R⁷ and R^{7′} are taken together to form an oxo moiety.

In certain embodiments, the present invention provides a compound wherein Ring E is a 6 membered aromatic ring having 1-2 heteroatoms independently selected from nitrogen, oxygen, or sulfur, and wherein R³ and R⁸ of Ring D are each independently selected from halogen, R, OR, or a suitably protected hydroxyl group, and wherein R⁷ and R^{7′} of Ring D are each independently selected from halogen, R, OR, a suitably protected hydroxyl group, or R⁷ and R^{7′} are taken together to form an oxo moiety.

In some embodiments, the present invention provides a compound wherein Ring E is a 7 membered saturated or partially unsaturated carbocycle, and wherein R³ and R⁸ of Ring D are each independently selected from halogen, CN, R, OR, a suitably protected hydroxyl group, SR, a suitably protected thiol group, S(O)R, SO₂R, OSO₂R, N(R)₂, a suitably protected amino group, N(R)C(O)R, N(R)C(O)C(O)R, N(R)C(O)N(R)₂, N(R)C(O)OR, C(O)OR, OC(O)R, C(O)N(R)₂, or OC(O)N(R)₂, and wherein R⁷ and R^{7′} of Ring D are each independently selected from halogen, CN, N₃, R, OR, a suitably protected hydroxyl group, SR, a suitably protected thiol group, SO₂R, OSO₂R, N(R)₂, a suitably protected amino group, N(R)C(O)R, N(R)C(O)C(O)R, N(R)C(O)N(R)₂, N(R)C(O)OR, C(O)OR, OC(O)R, C(O)N(R)₂, or OC(O)N(R)₂, or R⁷ and R^{7′} are taken together to form an oxo moiety.

In certain embodiments, the present invention provides a compound wherein Ring E is a 7 membered saturated carbocycle, wherein R³ and R⁸ of Ring D are each independently selected from halogen, R, OR, or a suitably protected hydroxyl group, and wherein R⁷ and R^{7′} of Ring D are each independently selected from halogen, R, OR, a suitably protected hydroxyl group, or R⁷ and R^{7′} are taken together to form an oxo moiety.

In certain embodiments, the present invention provides a compound wherein Ring E is a 7 membered partially unsaturated carbocycle, wherein R³ and R⁸ of Ring D are each independently selected from halogen, R, OR, or a suitably protected hydroxyl group, and wherein R⁷ and R^{7′} of Ring D are each independently selected from halogen, R, OR, a suitably protected hydroxyl group, or R⁷ and R^{7′} are taken together to form an oxo moiety.

In some embodiments, the present invention provides a compound wherein Ring E is a 7 membered saturated or partially unsaturated ring having 1-2 heteroatoms independently selected from nitrogen, oxygen, or sulfur, and wherein R³ and R⁸ of Ring D are each independently selected from halogen, CN, R, OR, a suitably protected hydroxyl group, SR, a suitably protected thiol group, S(O)R, SO₂R, OSO₂R, N(R)₂, a suitably protected amino group, N(R)C(O)R, N(R)C(O)C(O)R, N(R)C(O)N(R)₂, N(R)C(O)OR, C(O)OR, OC(O)R, C(O)N(R)₂, or OC(O)N(R)₂, and wherein R⁷ and R^{7′} of Ring D are each independently selected from halogen, CN, N₃, R, OR, a suitably protected hydroxyl group, SR, a suitably protected thiol group, SO₂R, OSO₂R, N(R)₂, a suitably protected amino group, N(R)C(O)R, N(R)C(O)C(O)R, N(R)C(O)N(R)₂, N(R)C(O)OR, C(O)OR, OC(O)R, C(O)N(R)₂, or OC(O)N(R)₂, or R⁷ and R^{7′} are taken together to form an oxo moiety.

In certain embodiments, the present invention provides a compound wherein Ring E is a 7 membered saturated ring having 1-2 heteroatoms independently selected from nitrogen, oxygen, or sulfur, and wherein R³ and R⁸ of Ring D are each independently selected from halogen, R, OR, or a suitably protected hydroxyl group, and wherein R⁷ and R^{7′} of Ring D are each independently selected from halogen, R, OR, a suitably protected hydroxyl group, or R⁷ and R^{7′} are taken together to form an oxo moiety.

In certain embodiments, the present invention provides a compound wherein Ring E is a 7 membered partially unsaturated ring having 1-2 heteroatoms independently selected from nitrogen, oxygen, or sulfur, and wherein R³ and R⁸ of Ring D are each independently selected from halogen, R, OR, or a suitably protected hydroxyl group, and wherein R⁷ and R^{7′} of Ring D are each independently selected from halogen, R, OR, a suitably protected hydroxyl group, or R⁷ and R^{7′} are taken together to form an oxo moiety.

### Exemplary Ring E/Q-R¹⁰ Combinations

In some embodiments, the present invention provides a compound wherein Ring E is a 4 membered saturated or partially unsaturated carbocycle, wherein Q is a valence bond, and wherein R¹⁰ of the Q-R¹⁰ moiety is selected from the group consisting of hydrogen, halogen, an optionally substituted C₁₋₁₀ aliphatic, a suitably protected hydroxyl group, a suitably protected thiol group, a suitably protected amino group, an optionally substituted 3-8 membered saturated, partially unsaturated, or aryl monocyclic ring having 0-4 heteroatoms independently selected from nitrogen, oxygen, or sulfur, an optionally substituted 8-10 membered saturated, partially unsaturated, or aryl bicyclic ring having 0-4 heteroatoms independently selected from nitrogen, oxygen, or sulfur, a detectable moiety, a polymer residue, a peptide, a sugar-containing or sugar-like moiety, or:

wherein when R¹⁰ is a ring, R¹⁰ is optionally substituted at any substitutable carbon with 1-5 R¹¹ and at any substitutable nitrogen with R¹²;

each R¹¹ is independently halogen, R, OR, SR, N(R)₂, N(R)C(O)R, N(R)C(O)OR, N(R)C(O)N(R)₂, N(R)SO₂R, N(R)SO₂OR, S(O)R, SO₂R, OSO₂R, C(O)R, CO₂R, OCO₂R, C(O)N(R)₂, or OC(O)N(R)₂, or wherein:

two R¹¹ are optionally taken together to form an optionally substituted 3-8 membered saturated or partially unsaturated fused or spirofused ring having 0-4 heteroatoms independently selected from nitrogen, oxygen, or sulfur; and

each R¹² is independently R, OR, S(O)R, SO₂R, OSO₂R, C(O)R, CO₂R, OCO₂R, C(O)N(R)₂, or OC(O)N(R)₂, or wherein:

R¹² and R¹¹ are optionally taken together to form an oxo moiety or an optionally substituted 3-8 membered saturated or partially unsaturated fused ring having 0-4 heteroatoms independently selected from nitrogen, oxygen, or sulfur.

In certain embodiments, the present invention provides a compound wherein Ring E is a 4 membered saturated carbocycle, wherein Q is a valence bond, and wherein R¹⁰ of the Q-R¹⁰ moiety is selected from the group consisting of hydrogen, halogen, a suitably protected hydroxyl group, a suitably protected thiol group, or a suitably protected amino group.

In certain embodiments, the present invention provides a compound wherein Ring E is a 4 membered saturated carbocycle, wherein Q is a valence bond, and wherein R¹⁰ of the Q-R¹⁰ moiety is a ring optionally substituted at any substitutable carbon with 1-5 R¹¹ and at any substitutable nitrogen with R¹², wherein each R¹¹ is independently selected from halogen, R, OR, SR, N(R)₂, N(R)C(O)R, N(R)C(O)OR, N(R)C(O)N(R)₂, N(R)SO₂R, N(R)SO₂OR, S(O)R, SO₂R, OSO₂R, C(O)R, CO₂R, OCO₂R, C(O)N(R)₂, or OC(O)N(R)₂.

In certain embodiments, the present invention provides a compound wherein Ring E is a 4 membered saturated carbocycle, wherein Q is a valence bond, and wherein R¹⁰ of the Q-R¹⁰ moiety is a sugar-containing or sugar-like moiety.

In some embodiments, the present invention provides a compound wherein Ring E is a 4 membered saturated or partially unsaturated ring having 1-2 heteroatoms independently selected from nitrogen, oxygen, or sulfur, and wherein Q is a valence bond and wherein R¹⁰ of the Q-R¹⁰ moiety is selected from the group consisting of hydrogen, halogen, an optionally substituted C₁₋₁₀ aliphatic, a suitably protected hydroxyl group, a suitably protected thiol group, a suitably protected amino group, an optionally substituted 3-8 membered saturated, partially unsaturated, or aryl monocyclic ring having 0-4 heteroatoms independently selected from nitrogen, oxygen, or sulfur, an optionally substituted 8-10 membered saturated, partially unsaturated, or aryl bicyclic ring having 0-4 heteroatoms independently selected from nitrogen, oxygen, or sulfur, a detectable moiety, a polymer residue, a peptide, a sugar-containing or sugar-like moiety, or:

wherein when R¹⁰ is a ring, R¹⁰ is optionally substituted at any substitutable carbon with 1-5 R¹¹ and at any substitutable nitrogen with R¹²;

each R¹¹ is independently halogen, R, OR, SR, N(R)₂, N(R)C(O)R, N(R)C(O)OR, N(R)C(O)N(R)₂, N(R)SO₂R, N(R)SO₂OR, S(O)R, SO₂R, OSO₂R, C(O)R, CO₂R, OCO₂R, C(O)N(R)₂, or OC(O)N(R)₂, or wherein:

two R¹¹ are optionally taken together to form an oxo moiety or an optionally substituted 3-8 membered saturated or partially unsaturated fused or spirofused ring having 0-4 heteroatoms independently selected from nitrogen, oxygen, or sulfur; and

each R¹² is independently R, OR, S(O)R, SO₂R, OSO₂R, C(O)R, CO₂R, OCO₂R, C(O)N(R)₂, or OC(O)N(R)₂, or wherein:

R¹² and R¹¹ are optionally taken together to form an optionally substituted 3-8 membered saturated or partially unsaturated fused ring having 0-4 heteroatoms independently selected from nitrogen, oxygen, or sulfur.

In certain embodiments, the present invention provides a compound wherein Ring E is a 4 membered saturated ring having 1-2 heteroatoms independently selected from nitrogen, oxygen, or sulfur, wherein Q is a valence bond, and wherein R¹⁰ of the Q-R¹⁰ moiety is selected from the group consisting of hydrogen, halogen, a suitably protected hydroxyl group, a suitably protected thiol group, or a suitably protected amino group.

In certain embodiments, the present invention provides a compound wherein Ring E is a 4 membered saturated ring having 1-2 heteroatoms independently selected from nitrogen, oxygen, or sulfur, wherein Q is a valence bond, and wherein R¹⁰ of the Q-R¹⁰ moiety is a ring optionally substituted at any substitutable carbon with 1-5 R¹¹ and at any substitutable nitrogen with R¹², wherein each R¹¹ is independently selected from halogen, R, OR, SR, N(R)₂, N(R)C(O)R, N(R)C(O)OR, N(R)C(O)N(R)₂, N(R)SO₂R, N(R)SO₂OR, S(O)R, SO₂R, OSO₂R, C(O)R, CO₂R, OCO₂R, C(O)N(R)₂, or OC(O)N(R)₂.

In certain embodiments, the present invention provides a compound wherein Ring E is a 4 membered saturated ring having 1-2 heteroatoms independently selected from nitrogen, oxygen, or sulfur, wherein Q is a valence bond, and wherein R¹⁰ of the Q-R¹⁰ moiety is a sugar-containing or sugar-like moiety.

In some embodiments, the present invention provides a compound wherein Ring E is a 5 membered saturated or partially unsaturated carbocycle, wherein Q is a valence bond, and wherein R¹⁰ of the Q-R¹⁰ moiety is selected from the group consisting of hydrogen, halogen, an optionally substituted C₁₋₁₀ aliphatic, a suitably protected hydroxyl group, a suitably protected thiol group, a suitably protected amino group, an optionally substituted 3-8 membered saturated, partially unsaturated, or aryl monocyclic ring having 0-4 heteroatoms independently selected from nitrogen, oxygen, or sulfur, an optionally substituted 8-10 membered saturated, partially unsaturated, or aryl bicyclic ring having 0-4 heteroatoms independently selected from nitrogen, oxygen, or sulfur, a detectable moiety, a polymer residue, a peptide, a sugar-containing or sugar-like moiety, or:

wherein when R¹⁰ is a ring, R¹⁰ is optionally substituted at any substitutable carbon with 1-5 R¹¹ and at any substitutable nitrogen with R¹²;

each R¹¹ is independently halogen, R, OR, SR, N(R)₂, N(R)C(O)R, N(R)C(O)OR, N(R)C(O)N(R)₂, N(R)SO₂R, N(R)SO₂OR, S(O)R, SO₂R, OSO₂R, C(O)R, CO₂R, OCO₂R, C(O)N(R)₂, or OC(O)N(R)₂, or wherein:

two R¹¹ are optionally taken together to form an oxo moiety or an optionally substituted 3-8 membered saturated or partially unsaturated fused or spirofused ring having 0-4 heteroatoms independently selected from nitrogen, oxygen, or sulfur; and

each R¹² is independently R, OR, S(O)R, SO₂R, OSO₂R, C(O)R, CO₂R, OCO₂R, C(O)N(R)₂, or OC(O)N(R)₂, or wherein:

R¹² and R¹¹ are optionally taken together to form an optionally substituted 3-8 membered saturated or partially unsaturated fused ring having 0-4 heteroatoms independently selected from nitrogen, oxygen, or sulfur.

In certain embodiments, the present invention provides a compound wherein Ring E is a 5 membered saturated carbocycle, wherein Q is a valence bond, and wherein R¹⁰ of the Q-R¹⁰ moiety is selected from the group consisting of hydrogen, halogen, a suitably protected hydroxyl group, a suitably protected thiol group, or a suitably protected amino group.

In certain embodiments, the present invention provides a compound wherein Ring E is a 5 membered saturated carbocycle, wherein Q is a valence bond, and wherein R¹⁰ of the Q-R¹⁰ moiety is a ring optionally substituted at any substitutable carbon with 1-5 R¹¹ and at any substitutable nitrogen with R¹², wherein each R¹¹ is independently selected from halogen, R, OR, SR, N(R)₂, N(R)C(O)R, N(R)C(O)OR, N(R)C(O)N(R)₂, N(R)SO₂R, N(R)SO₂OR, S(O)R, SO₂R, OSO₂R, C(O)R, CO₂R, OCO₂R, C(O)N(R)₂, or OC(O)N(R)₂.

In certain embodiments, the present invention provides a compound wherein Ring E is a 5 membered saturated carbocycle, wherein Q is a valence bond, and wherein R¹⁰ of the Q-R¹⁰ moiety is a sugar-containing or sugar-like moiety.

In certain embodiments, the present invention provides a compound wherein Ring E is a 5 membered partially unsaturated carbocycle, wherein Q is a valence bond, and wherein R¹⁰ of the Q-R¹⁰ moiety is selected from the group consisting of hydrogen, halogen, a suitably protected hydroxyl group, a suitably protected thiol group, or a suitably protected amino group.

In certain embodiments, the present invention provides a compound wherein Ring E is a 5 membered partially unsaturated carbocycle, wherein Q is a valence bond, and wherein R¹⁰ of the Q-R¹⁰ moiety is a ring optionally substituted at any substitutable carbon with 1-5 R¹¹ and at any substitutable nitrogen with R¹², wherein each R¹¹ is independently selected from halogen, R, OR, SR, N(R)₂, N(R)C(O)R, N(R)C(O)OR, N(R)C(O)N(R)₂, N(R)SO₂R, N(R)SO₂OR, S(O)R, SO₂R, OSO₂R, C(O)R, CO₂R, OCO₂R, C(O)N(R)₂, or OC(O)N(R)₂.

In certain embodiments, the present invention provides a compound wherein Ring E is a 5 membered partially unsaturated carbocycle, wherein Q is a valence bond, and wherein R¹⁰ of the Q-R¹⁰ moiety is a sugar-containing or sugar-like moiety.

In some embodiments, the present invention provides a compound wherein Ring E is a 5 membered saturated, partially unsaturated, or aromatic ring having 1-2 heteroatoms independently selected from nitrogen, oxygen, or sulfur, and wherein Q is a valence bond and wherein R¹⁰ of the Q-R¹⁰ moiety is selected from the group consisting of hydrogen, halogen, an optionally substituted C₁₋₁₀ aliphatic, a suitably protected hydroxyl group, a suitably protected thiol group, a suitably protected amino group, an optionally substituted 3-8 membered saturated, partially unsaturated, or aryl monocyclic ring having 0-4 heteroatoms independently selected from nitrogen, oxygen, or sulfur, an optionally substituted 8-10 membered saturated, partially unsaturated, or aryl bicyclic ring having 0-4 heteroatoms independently selected from nitrogen, oxygen, or sulfur, a detectable moiety, a polymer residue, a peptide, a sugar-containing or sugar-like moiety, or:

wherein when R¹⁰ is a ring, R¹⁰ is optionally substituted at any substitutable carbon with 1-5 R¹¹ and at any substitutable nitrogen with R¹²;

each R¹¹ is independently halogen, R, OR, SR, N(R)₂, N(R)C(O)R, N(R)C(O)OR, N(R)C(O)N(R)₂, N(R)SO₂R, N(R)SO₂OR, S(O)R, SO₂R, OSO₂R, C(O)R, CO₂R, OCO₂R, C(O)N(R)₂, or OC(O)N(R)₂, or wherein:

two R¹¹ are optionally taken together to form an oxo moiety or an optionally substituted 3-8 membered saturated or partially unsaturated fused or spirofused ring having 0-4 heteroatoms independently selected from nitrogen, oxygen, or sulfur; and

each R¹² is independently R, OR, S(O)R, SO₂R, OSO₂R, C(O)R, CO₂R, OCO₂R, C(O)N(R)₂, or OC(O)N(R)₂, or wherein:

R¹² and R¹¹ are optionally taken together to form an optionally substituted 3-8 membered saturated or partially unsaturated fused ring having 0-4 heteroatoms independently selected from nitrogen, oxygen, or sulfur.

In certain embodiments, the present invention provides a compound wherein Ring E is a 5 membered saturated ring having 1-2 heteroatoms independently selected from nitrogen, oxygen, or sulfur, wherein Q is a valence bond, and wherein R¹⁰ of the Q-R¹⁰ moiety is selected from the group consisting of hydrogen, halogen, a suitably protected hydroxyl group, a suitably protected thiol group, or a suitably protected amino group.

In certain embodiments, the present invention provides a compound wherein Ring E is a 5 membered saturated ring having 1-2 heteroatoms independently selected from nitrogen, oxygen, or sulfur, wherein Q is a valence bond, and wherein R¹⁰ of the Q-R¹⁰ moiety is a ring optionally substituted at any substitutable carbon with 1-5 R¹¹ and at any substitutable nitrogen with R¹², wherein each R¹¹ is independently selected from halogen, R, OR, SR, N(R)₂, N(R)C(O)R, N(R)C(O)OR, N(R)C(O)N(R)₂, N(R)SO₂R, N(R)SO₂OR, S(O)R, SO₂R, OSO₂R, C(O)R, CO₂R, OCO₂R, C(O)N(R)₂, or OC(O)N(R)₂.

In certain embodiments, the present invention provides a compound wherein Ring E is a 5 membered saturated ring having 1-2 heteroatoms independently selected from nitrogen, oxygen, or sulfur, wherein Q is a valence bond, and wherein R¹⁰ of the Q-R¹⁰ moiety is a sugar-containing or sugar-like moiety.

In certain embodiments, the present invention provides a compound wherein Ring E is a 5 membered partially unsaturated ring having 1-2 heteroatoms independently selected from nitrogen, oxygen, or sulfur, wherein Q is a valence bond, and wherein R¹⁰ of the Q-R¹⁰ moiety is selected from the group consisting of hydrogen, halogen, a suitably protected hydroxyl group, a suitably protected thiol group, or a suitably protected amino group.

In certain embodiments, the present invention provides a compound wherein Ring E is a 5 membered partially unsaturated ring having 1-2 heteroatoms independently selected from nitrogen, oxygen, or sulfur, wherein Q is a valence bond, and wherein R¹⁰ of the Q-R¹⁰ moiety is a ring optionally substituted at any substitutable carbon with 1-5 R¹¹ and at any substitutable nitrogen with R¹², wherein each R¹¹ is independently selected from halogen, R, OR, SR, N(R)₂, N(R)C(O)R, N(R)C(O)OR, N(R)C(O)N(R)₂, N(R)SO₂R, N(R)SO₂OR, S(O)R, SO₂R, OSO₂R, C(O)R, CO₂R, OCO₂R, C(O)N(R)₂, or OC(O)N(R)₂.

In certain embodiments, the present invention provides a compound wherein Ring E is a 5 membered partially unsaturated ring having 1-2 heteroatoms independently selected from nitrogen, oxygen, or sulfur, wherein Q is a valence bond, and wherein R¹⁰ of the Q-R¹⁰ moiety is a sugar-containing or sugar-like moiety.

In certain embodiments, the present invention provides a compound wherein Ring E is a 5 membered aromatic ring having 1-2 heteroatoms independently selected from nitrogen, oxygen, or sulfur, wherein Q is a valence bond, and wherein R¹⁰ of the Q-R¹⁰ moiety is selected from the group consisting of hydrogen, halogen, a suitably protected hydroxyl group, a suitably protected thiol group, or a suitably protected amino group.

In certain embodiments, the present invention provides a compound wherein Ring E is a 5 membered aromatic ring having 1-2 heteroatoms independently selected from nitrogen, oxygen, or sulfur, wherein Q is a valence bond, and wherein R¹⁰ of the Q-R¹⁰ moiety is a ring optionally substituted at any substitutable carbon with 1-5 R¹¹ and at any substitutable nitrogen with R¹², wherein each R¹¹ is independently selected from halogen, R, OR, SR, N(R)₂, N(R)C(O)R, N(R)C(O)OR, N(R)C(O)N(R)₂, N(R)SO₂R, N(R)SO₂OR, S(O)R, SO₂R, OSO₂R, C(O)R, CO₂R, OCO₂R, C(O)N(R)₂, or OC(O)N(R)₂.

In certain embodiments, the present invention provides a compound wherein Ring E is a 5 membered aromatic ring having 1-2 heteroatoms independently selected from nitrogen, oxygen, or sulfur, wherein Q is a valence bond, and wherein R¹⁰ of the Q-R¹⁰ moiety is a sugar-containing or sugar-like moiety.

In some embodiments, the present invention provides a compound wherein Ring E is a 6 membered saturated, partially unsaturated, or aromatic carbocycle, wherein Q is a valence bond, and wherein R¹⁰ of the Q-R¹⁰ moiety is selected from the group consisting of hydrogen, halogen, an optionally substituted C₁₋₁₀ aliphatic, a suitably protected hydroxyl group, a suitably protected thiol group, a suitably protected amino group, an optionally substituted 3-8 membered saturated, partially unsaturated, or aryl monocyclic ring having 0-4 heteroatoms independently selected from nitrogen, oxygen, or sulfur, an optionally substituted 8-10 membered saturated, partially unsaturated, or aryl bicyclic ring having 0-4 heteroatoms independently selected from nitrogen, oxygen, or sulfur, a detectable moiety, a polymer residue, a peptide, a sugar-containing or sugar-like moiety, or:

wherein when R¹⁰ is a ring, R¹⁰ is optionally substituted at any substitutable carbon with 1-5 R¹¹ and at any substitutable nitrogen with R¹²;

each R¹¹ is independently halogen, R, OR, SR, N(R)₂, N(R)C(O)R, N(R)C(O)OR, N(R)C(O)N(R)₂, N(R)SO₂R, N(R)SO₂OR, S(O)R, SO₂R, OSO₂R, C(O)R, CO₂R, OCO₂R, C(O)N(R)₂, or OC(O)N(R)₂, or wherein:

two R¹¹ are optionally taken together to form an oxo moiety or an optionally substituted 3-8 membered saturated or partially unsaturated fused or spirofused ring having 0-4 heteroatoms independently selected from nitrogen, oxygen, or sulfur; and

each R¹² is independently R, OR, S(O)R, SO₂R, OSO₂R, C(O)R, CO₂R, OCO₂R, C(O)N(R)₂, or OC(O)N(R)₂, or wherein:

R¹² and R¹¹ are optionally taken together to form an optionally substituted 3-8 membered saturated or partially unsaturated fused ring having 0-4 heteroatoms independently selected from nitrogen, oxygen, or sulfur.

In certain embodiments, the present invention provides a compound wherein Ring E is a 6 membered saturated carbocycle, wherein Q is a valence bond, and wherein R¹⁰ of the Q-R¹⁰ moiety is selected from the group consisting of hydrogen, halogen, a suitably protected hydroxyl group, a suitably protected thiol group, or a suitably protected amino group.

In certain embodiments, the present invention provides a compound wherein Ring E is a 6 membered saturated carbocycle, wherein Q is a valence bond, and wherein R¹⁰ of the Q-R¹⁰ moiety is a ring optionally substituted at any substitutable carbon with 1-5 R¹¹ and at any substitutable nitrogen with R¹², wherein each R¹¹ is independently selected from halogen, R, OR, SR, N(R)₂, N(R)C(O)R, N(R)C(O)OR, N(R)C(O)N(R)₂, N(R)SO₂R, N(R)SO₂OR, S(O)R, SO₂R, OSO₂R, C(O)R, CO₂R, OCO₂R, C(O)N(R)₂, or OC(O)N(R)₂.

In certain embodiments, the present invention provides a compound wherein Ring E is a 6 membered saturated carbocycle, wherein Q is a valence bond, and wherein R¹⁰ of the Q-R¹⁰ moiety is a sugar-containing or sugar-like moiety.

In certain embodiments, the present invention provides a compound wherein Ring E is a 6 membered partially unsaturated carbocycle, wherein Q is a valence bond, and wherein R¹⁰ of the Q-R¹⁰ moiety is selected from the group consisting of hydrogen, halogen, a suitably protected hydroxyl group, a suitably protected thiol group, or a suitably protected amino group.

In certain embodiments, the present invention provides a compound wherein Ring E is a 6 membered partially unsaturated carbocycle, wherein Q is a valence bond, and wherein R¹⁰ of the Q-R¹⁰ moiety is a ring optionally substituted at any substitutable carbon with 1-5 R¹¹ and at any substitutable nitrogen with R¹², wherein each R¹¹ is independently selected from halogen, R, OR, SR, N(R)₂, N(R)C(O)R, N(R)C(O)OR, N(R)C(O)N(R)₂, N(R)SO₂R, N(R)SO₂OR, S(O)R, SO₂R, OSO₂R, C(O)R, CO₂R, OCO₂R, C(O)N(R)₂, or OC(O)N(R)₂.

In certain embodiments, the present invention provides a compound wherein Ring E is a 6 membered partially unsaturated carbocycle, wherein Q is a valence bond, and wherein R¹⁰ of the Q-R¹⁰ moiety is a sugar-containing or sugar-like moiety.

In certain embodiments, the present invention provides a compound wherein Ring E is a 6 membered aromatic carbocycle, wherein Q is a valence bond, and wherein R¹⁰ of the Q-R¹⁰ moiety is selected from the group consisting of hydrogen, halogen, a suitably protected hydroxyl group, a suitably protected thiol group, or a suitably protected amino group.

In certain embodiments, the present invention provides a compound wherein Ring E is a 6 membered aromatic carbocycle, wherein Q is a valence bond, and wherein R¹⁰ of the Q-R¹⁰ moiety is a ring optionally substituted at any substitutable carbon with 1-5 R¹¹ and at any substitutable nitrogen with R¹², wherein each R¹¹ is independently selected from halogen, R, OR, SR, N(R)₂, N(R)C(O)R, N(R)C(O)OR, N(R)C(O)N(R)₂, N(R)SO₂R, N(R)SO₂OR, S(O)R, SO₂R, OSO₂R, C(O)R, CO₂R, OCO₂R, C(O)N(R)₂, or OC(O)N(R)₂.

In certain embodiments, the present invention provides a compound wherein Ring E is a 6 membered aromatic carbocycle, wherein Q is a valence bond, and wherein R¹⁰ of the Q-R¹⁰ moiety is a sugar-containing or sugar-like moiety.

In some embodiments, the present invention provides a compound wherein Ring E is a 6 membered saturated, partially unsaturated, or aromatic ring having 1-2 heteroatoms independently selected from nitrogen, oxygen, or sulfur, and wherein Q is a valence bond and wherein R¹⁰ of the Q-R¹⁰ moiety is selected from the group consisting of hydrogen, halogen, an optionally substituted C₁₋₁₀ aliphatic, a suitably protected hydroxyl group, a suitably protected thiol group, a suitably protected amino group, an optionally substituted 3-8 membered saturated, partially unsaturated, or aryl monocyclic ring having 0-4 heteroatoms independently selected from nitrogen, oxygen, or sulfur, an optionally substituted 8-10 membered saturated, partially unsaturated, or aryl bicyclic ring having 0-4 heteroatoms independently selected from nitrogen, oxygen, or sulfur, a detectable moiety, a polymer residue, a peptide, a sugar-containing or sugar-like moiety, or:

wherein when R¹⁰ is a ring, R¹⁰ is optionally substituted at any substitutable carbon with 1-5 R¹¹ and at any substitutable nitrogen with R¹²;

each R¹¹ is independently halogen, R, OR, SR, N(R)₂, N(R)C(O)R, N(R)C(O)OR, N(R)C(O)N(R)₂, N(R)SO₂R, N(R)SO₂OR, S(O)R, SO₂R, OSO₂R, C(O)R, CO₂R, OCO₂R, C(O)N(R)₂, or OC(O)N(R)₂, or wherein:

two R¹¹ are optionally taken together to form an oxo moiety or an optionally substituted 3-8 membered saturated or partially unsaturated fused or spirofused ring having 0-4 heteroatoms independently selected from nitrogen, oxygen, or sulfur; and

each R¹² is independently R, OR, S(O)R, SO₂R, OSO₂R, C(O)R, CO₂R, OCO₂R, C(O)N(R)₂, or OC(O)N(R)₂, or wherein:

R¹² and R¹¹ are optionally taken together to form an optionally substituted 3-8 membered saturated or partially unsaturated fused ring having 0-4 heteroatoms independently selected from nitrogen, oxygen, or sulfur.

In certain embodiments, the present invention provides a compound wherein Ring E is a 6 membered saturated ring having 1-2 heteroatoms independently selected from nitrogen, oxygen, or sulfur, wherein Q is a valence bond, and wherein R¹⁰ of the Q-R¹⁰ moiety is selected from the group consisting of hydrogen, halogen, a suitably protected hydroxyl group, a suitably protected thiol group, or a suitably protected amino group.

In certain embodiments, the present invention provides a compound wherein Ring E is a 6 membered saturated ring having 1-2 heteroatoms independently selected from nitrogen, oxygen, or sulfur, wherein Q is a valence bond, and wherein R¹⁰ of the Q-R¹⁰ moiety is a ring optionally substituted at any substitutable carbon with 1-5 R¹¹ and at any substitutable nitrogen with R¹², wherein each R¹¹ is independently selected from halogen, R, OR, SR, N(R)₂, N(R)C(O)R, N(R)C(O)OR, N(R)C(O)N(R)₂, N(R)SO₂R, N(R)SO₂OR, S(O)R, SO₂R, OSO₂R, C(O)R, CO₂R, OCO₂R, C(O)N(R)₂, or OC(O)N(R)₂.

In certain embodiments, the present invention provides a compound wherein Ring E is a 6 membered saturated ring having 1-2 heteroatoms independently selected from nitrogen, oxygen, or sulfur, wherein Q is a valence bond, and wherein R¹⁰ of the Q-R¹⁰ moiety is a sugar-containing or sugar-like moiety.

In certain embodiments, the present invention provides a compound wherein Ring E is a 6 membered partially unsaturated ring having 1-2 heteroatoms independently selected from nitrogen, oxygen, or sulfur, wherein Q is a valence bond, and wherein R¹⁰ of the Q-R¹⁰ moiety is selected from the group consisting of hydrogen, halogen, a suitably protected hydroxyl group, a suitably protected thiol group, or a suitably protected amino group.

In certain embodiments, the present invention provides a compound wherein Ring E is a 6 membered partially unsaturated ring having 1-2 heteroatoms independently selected from nitrogen, oxygen, or sulfur, wherein Q is a valence bond, and wherein R¹⁰ of the Q-R¹⁰ moiety is a ring optionally substituted at any substitutable carbon with 1-5 R¹¹ and at any substitutable nitrogen with R¹², wherein each R¹¹ is independently selected from halogen, R, OR, SR, N(R)₂, N(R)C(O)R, N(R)C(O)OR, N(R)C(O)N(R)₂, N(R)SO₂R, N(R)SO₂OR, S(O)R, SO₂R, OSO₂R, C(O)R, CO₂R, OCO₂R, C(O)N(R)₂, or OC(O)N(R)₂.

In certain embodiments, the present invention provides a compound wherein Ring E is a 6 membered partially unsaturated ring having 1-2 heteroatoms independently selected from nitrogen, oxygen, or sulfur, wherein Q is a valence bond, and wherein R¹⁰ of the Q-R¹⁰ moiety is a sugar-containing or sugar-like moiety.

In certain embodiments, the present invention provides a compound wherein Ring E is a 6 membered aromatic ring having 1-2 heteroatoms independently selected from nitrogen, oxygen, or sulfur, wherein Q is a valence bond, and wherein R¹⁰ of the Q-R¹⁰ moiety is selected from the group consisting of hydrogen, halogen, a suitably protected hydroxyl group, a suitably protected thiol group, or a suitably protected amino group.

In certain embodiments, the present invention provides a compound wherein Ring E is a 6 membered aromatic ring having 1-2 heteroatoms independently selected from nitrogen, oxygen, or sulfur, wherein Q is a valence bond, and wherein R¹⁰ of the Q-R¹⁰ moiety is a ring optionally substituted at any substitutable carbon with 1-5 R¹¹ and at any substitutable nitrogen with R¹², wherein each R¹¹ is independently selected from halogen, R, OR, SR, N(R)₂, N(R)C(O)R, N(R)C(O)OR, N(R)C(O)N(R)₂, N(R)SO₂R, N(R)SO₂OR, S(O)R, SO₂R, OSO₂R, C(O)R, CO₂R, OCO₂R, C(O)N(R)₂, or OC(O)N(R)₂.

In certain embodiments, the present invention provides a compound wherein Ring E is a 6 membered aromatic ring having 1-2 heteroatoms independently selected from nitrogen, oxygen, or sulfur, wherein Q is a valence bond, and wherein R¹⁰ of the Q-R¹⁰ moiety is a sugar-containing or sugar-like moiety.

In some embodiments, the present invention provides a compound wherein Ring E is a 7 membered saturated or partially unsaturated carbocycle, wherein Q is a valence bond, and wherein R¹⁰ of the Q-R¹⁰ moiety is selected from the group consisting of hydrogen, halogen, an optionally substituted C₁₋₁₀ aliphatic, a suitably protected hydroxyl group, a suitably protected thiol group, a suitably protected amino group, an optionally substituted 3-8 membered saturated, partially unsaturated, or aryl monocyclic ring having 0-4 heteroatoms independently selected from nitrogen, oxygen, or sulfur, an optionally substituted 8-10 membered saturated, partially unsaturated, or aryl bicyclic ring having 0-4 heteroatoms independently selected from nitrogen, oxygen, or sulfur, a detectable moiety, a polymer residue, a peptide, a sugar-containing or sugar-like moiety, or:

wherein when R¹⁰ is a ring, R¹⁰ is optionally substituted at any substitutable carbon with 1-5 R¹¹ and at any substitutable nitrogen with R¹²;

each R¹¹ is independently halogen, R, OR, SR, N(R)₂, N(R)C(O)R, N(R)C(O)OR, N(R)C(O)N(R)₂, N(R)SO₂R, N(R)SO₂OR, S(O)R, SO₂R, OSO₂R, C(O)R, CO₂R, OCO₂R, C(O)N(R)₂, or OC(O)N(R)₂, or wherein:

two R¹¹ are optionally taken together to form an oxo moiety or an optionally substituted 3-8 membered saturated or partially unsaturated fused or spirofused ring having 0-4 heteroatoms independently selected from nitrogen, oxygen, or sulfur; and

each R¹² is independently R, OR, S(O)R, SO₂R, OSO₂R, C(O)R, CO₂R, OCO₂R, C(O)N(R)₂, or OC(O)N(R)₂, or wherein:

R¹² and R¹¹ are optionally taken together to form an optionally substituted 3-8 membered saturated or partially unsaturated fused ring having 0-4 heteroatoms independently selected from nitrogen, oxygen, or sulfur.

In certain embodiments, the present invention provides a compound wherein Ring E is a 7 membered saturated carbocycle, wherein Q is a valence bond, and wherein R¹⁰ of the Q-R¹⁰ moiety is selected from the group consisting of hydrogen, halogen, a suitably protected hydroxyl group, a suitably protected thiol group, or a suitably protected amino group.

In certain embodiments, the present invention provides a compound wherein Ring E is a 7 membered saturated carbocycle, wherein Q is a valence bond, and wherein R¹⁰ of the Q-R¹⁰ moiety is a ring optionally substituted at any substitutable carbon with 1-5 R¹¹ and at any substitutable nitrogen with R¹², wherein each R¹¹ is independently selected from halogen, R, OR, SR, N(R)₂, N(R)C(O)R, N(R)C(O)OR, N(R)C(O)N(R)₂, N(R)SO₂R, N(R)SO₂OR, S(O)R, SO₂R, OSO₂R, C(O)R, CO₂R, OCO₂R, C(O)N(R)₂, or OC(O)N(R)₂.

In certain embodiments, the present invention provides a compound wherein Ring E is a 7 membered saturated carbocycle, wherein Q is a valence bond, and wherein R¹⁰ of the Q-R¹⁰ moiety is a sugar-containing or sugar-like moiety.

In certain embodiments, the present invention provides a compound wherein Ring E is a 7 membered partially unsaturated carbocycle, wherein Q is a valence bond, and wherein R¹⁰ of the Q-R¹⁰ moiety is selected from the group consisting of hydrogen, halogen, a suitably protected hydroxyl group, a suitably protected thiol group, or a suitably protected amino group.

In certain embodiments, the present invention provides a compound wherein Ring E is a 7 membered partially unsaturated carbocycle, wherein Q is a valence bond, and wherein R¹⁰ of the Q-R¹⁰ moiety is a ring optionally substituted at any substitutable carbon with 1-5 R¹¹ and at any substitutable nitrogen with R¹², wherein each R¹¹ is independently selected from halogen, R, OR, SR, N(R)₂, N(R)C(O)R, N(R)C(O)OR, N(R)C(O)N(R)₂, N(R)SO₂R, N(R)SO₂OR, S(O)R, SO₂R, OSO₂R, C(O)R, CO₂R, OCO₂R, C(O)N(R)₂, or OC(O)N(R)₂.

In certain embodiments, the present invention provides a compound wherein Ring E is a 7 membered partially unsaturated carbocycle, wherein Q is a valence bond, and wherein R¹⁰ of the Q-R¹⁰ moiety is a sugar-containing or sugar-like moiety.

In some embodiments, the present invention provides a compound wherein Ring E is a 7 membered saturated or partially unsaturated ring having 1-2 heteroatoms independently selected from nitrogen, oxygen, or sulfur, and wherein Q is a valence bond and wherein R¹⁰ of the Q-R¹⁰ moiety is selected from the group consisting of hydrogen, halogen, an optionally substituted C₁₋₁₀ aliphatic, a suitably protected hydroxyl group, a suitably protected thiol group, a suitably protected amino group, an optionally substituted 3-8 membered saturated, partially unsaturated, or aryl monocyclic ring having 0-4 heteroatoms independently selected from nitrogen, oxygen, or sulfur, an optionally substituted 8-10 membered saturated, partially unsaturated, or aryl bicyclic ring having 0-4 heteroatoms independently selected from nitrogen, oxygen, or sulfur, a detectable moiety, a polymer residue, a peptide, a sugar-containing or sugar-like moiety, or:

wherein when R¹⁰ is a ring, R¹⁰ is optionally substituted at any substitutable carbon with 1-5 R¹¹ and at any substitutable nitrogen with R¹²;

each R¹¹ is independently halogen, R, OR, SR, N(R)₂, N(R)C(O)R, N(R)C(O)OR, N(R)C(O)N(R)₂, N(R)SO₂R, N(R)SO₂OR, S(O)R, SO₂R, OSO₂R, C(O)R, CO₂R, OCO₂R, C(O)N(R)₂, or OC(O)N(R)₂, or wherein:

two R¹¹ are optionally taken together to form an oxo moiety or an optionally substituted 3-8 membered saturated or partially unsaturated fused or spirofused ring having 0-4 heteroatoms independently selected from nitrogen, oxygen, or sulfur; and

each R¹² is independently R, OR, S(O)R, SO₂R, OSO₂R, C(O)R, CO₂R, OCO₂R, C(O)N(R)₂, or OC(O)N(R)₂, or wherein:

R¹² and R¹¹ are optionally taken together to form an optionally substituted 3-8 membered saturated or partially unsaturated fused ring having 0-4 heteroatoms independently selected from nitrogen, oxygen, or sulfur.

In certain embodiments, the present invention provides a compound wherein Ring E is a 7 membered saturated ring having 1-2 heteroatoms independently selected from nitrogen, oxygen, or sulfur, wherein Q is a valence bond, and wherein R¹⁰ of the Q-R¹⁰ moiety is selected from the group consisting of hydrogen, halogen, a suitably protected hydroxyl group, a suitably protected thiol group, or a suitably protected amino group.

In certain embodiments, the present invention provides a compound wherein Ring E is a 7 membered saturated ring having 1-2 heteroatoms independently selected from nitrogen, oxygen, or sulfur, wherein Q is a valence bond, and wherein R¹⁰ of the Q-R¹⁰ moiety is a ring optionally substituted at any substitutable carbon with 1-5 R¹¹ and at any substitutable nitrogen with R¹², wherein each R¹¹ is independently selected from halogen, R, OR, SR, N(R)₂, N(R)C(O)R, N(R)C(O)OR, N(R)C(O)N(R)₂, N(R)SO₂R, N(R)SO₂OR, S(O)R, SO₂R, OSO₂R, C(O)R, CO₂R, OCO₂R, C(O)N(R)₂, or OC(O)N(R)₂.

In certain embodiments, the present invention provides a compound wherein Ring E is a 7 membered saturated ring having 1-2 heteroatoms independently selected from nitrogen, oxygen, or sulfur, wherein Q is a valence bond, and wherein R¹⁰ of the Q-R¹⁰ moiety is a sugar-containing or sugar-like moiety.

In certain embodiments, the present invention provides a compound wherein Ring E is a 7 membered partially unsaturated ring having 1-2 heteroatoms independently selected from nitrogen, oxygen, or sulfur, wherein Q is a valence bond, and wherein R¹⁰ of the Q-R¹⁰ moiety is selected from the group consisting of hydrogen, halogen, a suitably protected hydroxyl group, a suitably protected thiol group, or a suitably protected amino group.

In certain embodiments, the present invention provides a compound wherein Ring E is a 7 membered partially unsaturated ring having 1-2 heteroatoms independently selected from nitrogen, oxygen, or sulfur, wherein Q is a valence bond, and wherein R¹⁰ of the Q-R¹⁰ moiety is a ring optionally substituted at any substitutable carbon with 1-5 R¹¹ and at any substitutable nitrogen with R¹², wherein each R¹¹ is independently selected from halogen, R, OR, SR, N(R)₂, N(R)C(O)R, N(R)C(O)OR, N(R)C(O)N(R)₂, N(R)SO₂R, N(R)SO₂OR, S(O)R, SO₂R, OSO₂R, C(O)R, CO₂R, OCO₂R, C(O)N(R)₂, or OC(O)N(R)₂.

In certain embodiments, the present invention provides a compound wherein Ring E is a 7 membered partially unsaturated ring having 1-2 heteroatoms independently selected from nitrogen, oxygen, or sulfur, wherein Q is a valence bond, and wherein R¹⁰ of the Q-R¹⁰ moiety is a sugar-containing or sugar-like moiety.

In some embodiments, the present invention provides a compound wherein Ring E is a 4 membered saturated or partially unsaturated carbocycle, wherein Q is an optionally substituted C₁₋₁₀ alkylene chain wherein one, two, or three methylene units of Q are optionally and independently replaced by —O—, —N(R)—, —S—, —C(O)—, —OC(O)—, —C(O)O—, —OC(O)O—, —S(O)—, or —S(O)₂—, —OSO₂O—, —N(R)C(O)—, —C(O)NR—, —N(R)C(O)O—, —OC(O)NR—, —N(R)C(O)NR—, or —Cy-, wherein:

each —Cy- is independently a bivalent optionally substituted saturated, partially unsaturated, or aromatic monocyclic or bicyclic ring selected from a 6-10 membered arylene, a 5-10 membered heteroarylene having 1-4 heteroatoms independently selected from oxygen, nitrogen, or sulfur, a 3-8 membered carbocyclylene, or a 3-10 membered heterocyclylene having 1-4 heteroatoms independently selected from oxygen, nitrogen, or sulfur, and:

wherein R¹⁰ of the Q-R¹⁰ moiety is selected from the group consisting of hydrogen, halogen, an optionally substituted C₁₋₁₀ aliphatic, a suitably protected hydroxyl group, a suitably protected thiol group, a suitably protected amino group, an optionally substituted 3-8 membered saturated, partially unsaturated, or aryl monocyclic ring having 0-4 heteroatoms independently selected from nitrogen, oxygen, or sulfur, an optionally substituted 8-10 membered saturated, partially unsaturated, or aryl bicyclic ring having 0-4 heteroatoms independently selected from nitrogen, oxygen, or sulfur, a detectable moiety, a polymer residue, a peptide, a sugar-containing or sugar-like moiety, or:

wherein when R¹⁰ is a ring, R¹⁰ is optionally substituted at any substitutable carbon with 1-5 R¹¹ and at any substitutable nitrogen with R¹²;

each R¹¹ is independently halogen, R, OR, SR, N(R)₂, N(R)C(O)R, N(R)C(O)OR, N(R)C(O)N(R)₂, N(R)SO₂R, N(R)SO₂OR, S(O)R, SO₂R, OSO₂R, C(O)R, CO₂R, OCO₂R, C(O)N(R)₂, or OC(O)N(R)₂, or wherein:

two R¹¹ are optionally taken together to form an oxo moiety or an optionally substituted 3-8 membered saturated or partially unsaturated fused or spirofused ring having 0-4 heteroatoms independently selected from nitrogen, oxygen, or sulfur; and

each R¹² is independently R, OR, S(O)R, SO₂R, OSO₂R, C(O)R, CO₂R, OCO₂R, C(O)N(R)₂, or OC(O)N(R)₂, or wherein:

R¹² and R¹¹ are optionally taken together to form an optionally substituted 3-8 membered saturated or partially unsaturated fused ring having 0-4 heteroatoms independently selected from nitrogen, oxygen, or sulfur.

In certain embodiments, the present invention provides a compound wherein Ring E is a 4 membered saturated carbocycle, wherein Q is an optionally substituted C₁₋₁₀ alkylene chain wherein one, two, or three methylene units of Q are optionally and independently replaced by —O—, —N(R)—, —S—, or —Cy-, and wherein R¹⁰ of the Q-R¹⁰ moiety is selected from the group consisting of hydrogen, halogen, a suitably protected hydroxyl group, a suitably protected thiol group, or a suitably protected amino group.

In certain embodiments, the present invention provides a compound wherein Ring E is a 4 membered saturated carbocycle, wherein Q is an optionally substituted C₁₋₁₀ alkylene chain wherein one, two, or three methylene units of Q are optionally and independently replaced by —O—, —N(R)—, —S—, or —Cy-, and wherein R¹⁰ of the Q-R¹⁰ moiety is a ring optionally substituted at any substitutable carbon with 1-5 R¹¹ and at any substitutable nitrogen with R¹², wherein each R¹¹ is independently selected from halogen, R, OR, SR, N(R)₂, N(R)C(O)R, N(R)C(O)OR, N(R)C(O)N(R)₂, N(R)SO₂R, N(R)SO₂OR, S(O)R, SO₂R, OSO₂R, C(O)R, CO₂R, OCO₂R, C(O)N(R)₂, or OC(O)N(R)₂. In certain embodiments, the compound is as described above and R¹⁰ is an optionally substituted heterocycle. In certain embodiments, the compound is as described above and R¹⁰ is an optionally substituted 5-6 membered heterocycle with 1-3 heteroatoms. In certain embodiments, the compound is as described above and R¹⁰ is an optionally substituted 6 membered heterocycle with 2 heteroatoms. In certain embodiments, the compound is as described above and R¹⁰ is optionally substituted morpholine.

In certain embodiments, the present invention provides a compound wherein Ring E is a 4 membered saturated carbocycle, wherein Q is an optionally substituted C₁₋₁₀ alkylene chain wherein one, two, or three methylene units of Q are optionally and independently replaced by —O—, —N(R)—, —S—, or —Cy-, and wherein R¹⁰ of the Q-R¹⁰ moiety is a sugar-containing or sugar-like moiety.

In some embodiments, the present invention provides a compound wherein Ring E is a 4 membered saturated or partially unsaturated ring having 1-2 heteroatoms independently selected from nitrogen, oxygen, or sulfur, and wherein Q is an optionally substituted C₁₋₁₀ alkylene chain wherein one, two, or three methylene units of Q are optionally and independently replaced by —O—, —N(R)—, —S—, —C(O)—, —OC(O)—, —C(O)O—, —OC(O)O—, —S(O)—, or —S(O)₂—, —OSO₂O—, —N(R)C(O)—, —C(O)NR—, —N(R)C(O)O—, —OC(O)NR—, —N(R)C(O)NR—, or —Cy-, wherein:

each —Cy- is independently a bivalent optionally substituted saturated, partially unsaturated, or aromatic monocyclic or bicyclic ring selected from a 6-10 membered arylene, a 5-10 membered heteroarylene having 1-4 heteroatoms independently selected from oxygen, nitrogen, or sulfur, a 3-8 membered carbocyclylene, or a 3-10 membered heterocyclylene having 1-4 heteroatoms independently selected from oxygen, nitrogen, or sulfur, and:

wherein R¹⁰ of the Q-R¹⁰ moiety is selected from the group consisting of hydrogen, halogen, an optionally substituted C₁₋₁₀ aliphatic, a suitably protected hydroxyl group, a suitably protected thiol group, a suitably protected amino group, an optionally substituted 3-8 membered saturated, partially unsaturated, or aryl monocyclic ring having 0-4 heteroatoms independently selected from nitrogen, oxygen, or sulfur, an optionally substituted 8-10 membered saturated, partially unsaturated, or aryl bicyclic ring having 0-4 heteroatoms independently selected from nitrogen, oxygen, or sulfur, a detectable moiety, a polymer residue, a peptide, a sugar-containing or sugar-like moiety, or:

wherein when R¹⁰ is a ring, R¹⁰ is optionally substituted at any substitutable carbon with 1-5 R¹¹ and at any substitutable nitrogen with R¹²;

each R¹¹ is independently halogen, R, OR, SR, N(R)₂, N(R)C(O)R, N(R)C(O)OR, N(R)C(O)N(R)₂, N(R)SO₂R, N(R)SO₂OR, S(O)R, SO₂R, OSO₂R, C(O)R, CO₂R, OCO₂R, C(O)N(R)₂, or OC(O)N(R)₂, or wherein:

two R¹¹ are optionally taken together to form an oxo moiety or an optionally substituted 3-8 membered saturated or partially unsaturated fused or spirofused ring having 0-4 heteroatoms independently selected from nitrogen, oxygen, or sulfur; and

each R¹² is independently R, OR, S(O)R, SO₂R, OSO₂R, C(O)R, CO₂R, OCO₂R, C(O)N(R)₂, or OC(O)N(R)₂, or wherein:

R¹² and R¹¹ are optionally taken together to form an optionally substituted 3-8 membered saturated or partially unsaturated fused ring having 0-4 heteroatoms independently selected from nitrogen, oxygen, or sulfur.

In certain embodiments, the present invention provides a compound wherein Ring E is a 4 membered saturated ring having 1-2 heteroatoms independently selected from nitrogen, oxygen, or sulfur, wherein Q is an optionally substituted C₁₋₁₀ alkylene chain wherein one, two, or three methylene units of Q are optionally and independently replaced by —O—, —N(R)—, —S—, or —Cy-, and wherein R¹⁰ of the Q-R¹⁰ moiety is selected from the group consisting of hydrogen, halogen, a suitably protected hydroxyl group, a suitably protected thiol group, or a suitably protected amino group.

In certain embodiments, the present invention provides a compound wherein Ring E is a 4 membered saturated ring having 1-2 heteroatoms independently selected from nitrogen, oxygen, or sulfur, wherein Q is an optionally substituted C₁₋₁₀ alkylene chain wherein one, two, or three methylene units of Q are optionally and independently replaced by —O—, —N(R)—, —S—, or —Cy-, and wherein R¹⁰ of the Q-R¹⁰ moiety is a ring optionally substituted at any substitutable carbon with 1-5 R¹¹ and at any substitutable nitrogen with R¹², wherein each R¹¹ is independently selected from halogen, R, OR, SR, N(R)₂, N(R)C(O)R, N(R)C(O)OR, N(R)C(O)N(R)₂, N(R)SO₂R, N(R)SO₂OR, S(O)R, SO₂R, OSO₂R, C(O)R, CO₂R, OCO₂R, C(O)N(R)₂, or OC(O)N(R)₂. In certain embodiments, the compound is as described above and R¹⁰ is an optionally substituted heterocycle. In certain embodiments, the compound is as described above and R¹⁰ is an optionally substituted 5-6 membered heterocycle with 1-3 heteroatoms. In certain embodiments, the compound is as described above and R¹⁰ is an optionally substituted 6 membered heterocycle with 2 heteroatoms. In certain embodiments, the compound is as described above and R¹⁰ is optionally substituted morpholine.

In certain embodiments, the present invention provides a compound wherein Ring E is a 4 membered saturated ring having 1-2 heteroatoms independently selected from nitrogen, oxygen, or sulfur, wherein Q is an optionally substituted C₁₋₁₀ alkylene chain wherein one, two, or three methylene units of Q are optionally and independently replaced by —O—, —N(R)—, —S—, or —Cy-, and wherein R¹⁰ of the Q-R¹⁰ moiety is a sugar-containing or sugar-like moiety.

In some embodiments, the present invention provides a compound wherein Ring E is a 5 membered saturated or partially unsaturated carbocycle, wherein Q is an optionally substituted C₁₋₁₀ alkylene chain wherein one, two, or three methylene units of Q are optionally and independently replaced by —O—, —N(R)—, —S—, —C(O)—, —OC(O)—, —C(O)O—, —OC(O)O—, —S(O)—, or —S(O)₂—, —OSO₂O—, —N(R)C(O)—, —C(O)NR—, —N(R)C(O)O—, —OC(O)NR—, —N(R)C(O)NR—, or —Cy-, wherein:

each —Cy- is independently a bivalent optionally substituted saturated, partially unsaturated, or aromatic monocyclic or bicyclic ring selected from a 6-10 membered arylene, a 5-10 membered heteroarylene having 1-4 heteroatoms independently selected from oxygen, nitrogen, or sulfur, a 3-8 membered carbocyclylene, or a 3-10 membered heterocyclylene having 1-4 heteroatoms independently selected from oxygen, nitrogen, or sulfur, and:

wherein R¹⁰ of the Q-R¹⁰ moiety is selected from the group consisting of hydrogen, halogen, an optionally substituted C₁₋₁₀ aliphatic, a suitably protected hydroxyl group, a suitably protected thiol group, a suitably protected amino group, an optionally substituted 3-8 membered saturated, partially unsaturated, or aryl monocyclic ring having 0-4 heteroatoms independently selected from nitrogen, oxygen, or sulfur, an optionally substituted 8-10 membered saturated, partially unsaturated, or aryl bicyclic ring having 0-4 heteroatoms independently selected from nitrogen, oxygen, or sulfur, a detectable moiety, a polymer residue, a peptide, a sugar-containing or sugar-like moiety, or:

wherein when R¹⁰ is a ring, R¹⁰ is optionally substituted at any substitutable carbon with 1-5 R¹¹ and at any substitutable nitrogen with R¹²;

each R¹¹ is independently halogen, R, OR, SR, N(R)₂, N(R)C(O)R, N(R)C(O)OR, N(R)C(O)N(R)₂, N(R)SO₂R, N(R)SO₂OR, S(O)R, SO₂R, OSO₂R, C(O)R, CO₂R, OCO₂R, C(O)N(R)₂, or OC(O)N(R)₂, or wherein:

two R¹¹ are optionally taken together to form an oxo moiety or an optionally substituted 3-8 membered saturated or partially unsaturated fused or spirofused ring having 0-4 heteroatoms independently selected from nitrogen, oxygen, or sulfur; and

each R¹² is independently R, OR, S(O)R, SO₂R, OSO₂R, C(O)R, CO₂R, OCO₂R, C(O)N(R)₂, or OC(O)N(R)₂, or wherein:

R¹² and R¹¹ are optionally taken together to form an optionally substituted 3-8 membered saturated or partially unsaturated fused ring having 0-4 heteroatoms independently selected from nitrogen, oxygen, or sulfur.

In certain embodiments, the present invention provides a compound wherein Ring E is a 5 membered saturated carbocycle, wherein Q is an optionally substituted C₁₋₁₀ alkylene chain wherein one, two, or three methylene units of Q are optionally and independently replaced by —O—, —N(R)—, —S—, or —Cy-, and wherein R¹⁰ of the Q-R¹⁰ moiety is selected from the group consisting of hydrogen, halogen, a suitably protected hydroxyl group, a suitably protected thiol group, or a suitably protected amino group.

In certain embodiments, the present invention provides a compound wherein Ring E is a 5 membered saturated carbocycle, wherein Q is an optionally substituted C₁₋₁₀ alkylene chain wherein one, two, or three methylene units of Q are optionally and independently replaced by —O—, —N(R)—, —S—, or —Cy-, and wherein R¹⁰ of the Q-R¹⁰ moiety is a ring optionally substituted at any substitutable carbon with 1-5 R¹¹ and at any substitutable nitrogen with R¹², wherein each R¹¹ is independently selected from halogen, R, OR, SR, N(R)₂, N(R)C(O)R, N(R)C(O)OR, N(R)C(O)N(R)₂, N(R)SO₂R, N(R)SO₂OR, S(O)R, SO₂R, OSO₂R, C(O)R, CO₂R, OCO₂R, C(O)N(R)₂, or OC(O)N(R)₂. In certain embodiments, the compound is as described above and R¹⁰ is an optionally substituted heterocycle. In certain embodiments, the compound is as described above and R¹⁰ is an optionally substituted 5-6 membered heterocycle with 1-3 heteroatoms. In certain embodiments, the compound is as described above and R¹⁰ is an optionally substituted 6 membered heterocycle with 2 heteroatoms. In certain embodiments, the compound is as described above and R¹⁰ is optionally substituted morpholine.

In certain embodiments, the present invention provides a compound wherein Ring E is a 5 membered saturated carbocycle, wherein Q is an optionally substituted C₁₋₁₀ alkylene chain wherein one, two, or three methylene units of Q are optionally and independently replaced by —O—, —N(R)—, —S—, or —Cy-, and wherein R¹⁰ of the Q-R¹⁰ moiety is a sugar-containing or sugar-like moiety.

In certain embodiments, the present invention provides a compound wherein Ring E is a 5 membered partially unsaturated carbocycle, wherein Q is an optionally substituted C₁₋₁₀ alkylene chain wherein one, two, or three methylene units of Q are optionally and independently replaced by —O—, —N(R)—, —S—, or —Cy-, and wherein R¹⁰ of the Q-R¹⁰ moiety is selected from the group consisting of hydrogen, halogen, a suitably protected hydroxyl group, a suitably protected thiol group, or a suitably protected amino group.

In certain embodiments, the present invention provides a compound wherein Ring E is a 5 membered partially unsaturated carbocycle, wherein Q is an optionally substituted C₁₋₁₀ alkylene chain wherein one, two, or three methylene units of Q are optionally and independently replaced by —O—, —N(R)—, —S—, or —Cy-, and wherein R¹⁰ of the Q-R¹⁰ moiety is a ring optionally substituted at any substitutable carbon with 1-5 R¹¹ and at any substitutable nitrogen with R¹², wherein each R¹¹ is independently selected from halogen, R, OR, SR, N(R)₂, N(R)C(O)R, N(R)C(O)OR, N(R)C(O)N(R)₂, N(R)SO₂R, N(R)SO₂OR, S(O)R, SO₂R, OSO₂R, C(O)R, CO₂R, OCO₂R, C(O)N(R)₂, or OC(O)N(R)₂. In certain embodiments, the compound is as described above and R¹⁰ is an optionally substituted heterocycle. In certain embodiments, the compound is as described above and R¹⁰ is an optionally substituted 5-6 membered heterocycle with 1-3 heteroatoms. In certain embodiments, the compound is as described above and R¹⁰ is an optionally substituted 6 membered heterocycle with 2 heteroatoms. In certain embodiments, the compound is as described above and R¹⁰ is optionally substituted morpholine.

In certain embodiments, the present invention provides a compound wherein Ring E is a 5 membered partially unsaturated carbocycle, wherein Q is an optionally substituted C₁₋₁₀ alkylene chain wherein one, two, or three methylene units of Q are optionally and independently replaced by —O—, —N(R)—, —S—, or —Cy-, and wherein R¹⁰ of the Q-R¹⁰ moiety is a sugar-containing or sugar-like moiety.

In some embodiments, the present invention provides a compound wherein Ring E is a 5 membered saturated, partially unsaturated, or aromatic ring having 1-2 heteroatoms independently selected from nitrogen, oxygen, or sulfur, and wherein Q is an optionally substituted C₁₋₁₀ alkylene chain wherein one, two, or three methylene units of Q are optionally and independently replaced by —O—, —N(R)—, —S—, —C(O)—, —OC(O)—, —C(O)O—, —OC(O)O—, —S(O)—, or —S(O)₂—, —OSO₂O—, —N(R)C(O)—, —C(O)NR—, —N(R)C(O)O—, —OC(O)NR—, —N(R)C(O)NR—, or —Cy-, wherein:

each —Cy- is independently a bivalent optionally substituted saturated, partially unsaturated, or aromatic monocyclic or bicyclic ring selected from a 6-10 membered arylene, a 5-10 membered heteroarylene having 1-4 heteroatoms independently selected from oxygen, nitrogen, or sulfur, a 3-8 membered carbocyclylene, or a 3-10 membered heterocyclylene having 1-4 heteroatoms independently selected from oxygen, nitrogen, or sulfur, and:

wherein R¹⁰ of the Q-R¹⁰ moiety is selected from the group consisting of hydrogen, halogen, an optionally substituted C₁₋₁₀ aliphatic, a suitably protected hydroxyl group, a suitably protected thiol group, a suitably protected amino group, an optionally substituted 3-8 membered saturated, partially unsaturated, or aryl monocyclic ring having 0-4 heteroatoms independently selected from nitrogen, oxygen, or sulfur, an optionally substituted 8-10 membered saturated, partially unsaturated, or aryl bicyclic ring having 0-4 heteroatoms independently selected from nitrogen, oxygen, or sulfur, a detectable moiety, a polymer residue, a peptide, a sugar-containing or sugar-like moiety, or:

wherein when R¹⁰ is a ring, R¹⁰ is optionally substituted at any substitutable carbon with 1-5 R¹¹ and at any substitutable nitrogen with R¹²;

each R¹¹ is independently halogen, R, OR, SR, N(R)₂, N(R)C(O)R, N(R)C(O)OR, N(R)C(O)N(R)₂, N(R)SO₂R, N(R)SO₂OR, S(O)R, SO₂R, OSO₂R, C(O)R, CO₂R, OCO₂R, C(O)N(R)₂, or OC(O)N(R)₂, or wherein:

two R¹¹ are optionally taken together to form an oxo moiety or an optionally substituted 3-8 membered saturated or partially unsaturated fused or spirofused ring having 0-4 heteroatoms independently selected from nitrogen, oxygen, or sulfur; and

each R¹² is independently R, OR, S(O)R, SO₂R, OSO₂R, C(O)R, CO₂R, OCO₂R, C(O)N(R)₂, or OC(O)N(R)₂, or wherein:

R¹² and R¹¹ are optionally taken together to form an optionally substituted 3-8 membered saturated or partially unsaturated fused ring having 0-4 heteroatoms independently selected from nitrogen, oxygen, or sulfur.

In certain embodiments, the present invention provides a compound wherein Ring E is a 5 membered saturated ring having 1-2 heteroatoms independently selected from nitrogen, oxygen, or sulfur, wherein Q is an optionally substituted C₁₋₁₀ alkylene chain wherein one, two, or three methylene units of Q are optionally and independently replaced by —O—, —N(R)—, —S—, or —Cy-, and wherein R¹⁰ of the Q-R¹⁰ moiety is selected from the group consisting of hydrogen, halogen, a suitably protected hydroxyl group, a suitably protected thiol group, or a suitably protected amino group.

In certain embodiments, the present invention provides a compound wherein Ring E is a 5 membered saturated ring having 1-2 heteroatoms independently selected from nitrogen, oxygen, or sulfur, wherein Q is an optionally substituted C₁₋₁₀ alkylene chain wherein one, two, or three methylene units of Q are optionally and independently replaced by —O—, —N(R)—, —S—, or —Cy-, and wherein R¹⁰ of the Q-R¹⁰ moiety is a ring optionally substituted at any substitutable carbon with 1-5 R¹¹ and at any substitutable nitrogen with R¹², wherein each R¹¹ is independently selected from halogen, R, OR, SR, N(R)₂, N(R)C(O)R, N(R)C(O)OR, N(R)C(O)N(R)₂, N(R)SO₂R, N(R)SO₂OR, S(O)R, SO₂R, OSO₂R, C(O)R, CO₂R, OCO₂R, C(O)N(R)₂, or OC(O)N(R)₂. In certain embodiments, the compound is as described above and R¹⁰ is an optionally substituted heterocycle. In certain embodiments, the compound is as described above and R¹⁰ is an optionally substituted 5-6 membered heterocycle with 1-3 heteroatoms. In certain embodiments, the compound is as described above and R¹⁰ is an optionally substituted 6 membered heterocycle with 2 heteroatoms. In certain embodiments, the compound is as described above and R¹⁰ is optionally substituted morpholine.

In certain embodiments, the present invention provides a compound wherein Ring E is a 5 membered saturated ring having 1-2 heteroatoms independently selected from nitrogen, oxygen, or sulfur, wherein Q is an optionally substituted C₁₋₁₀ alkylene chain wherein one, two, or three methylene units of Q are optionally and independently replaced by —O—, —N(R)—, —S—, or —Cy-, and wherein R¹⁰ of the Q-R¹⁰ moiety is a sugar-containing or sugar-like moiety.

In certain embodiments, the present invention provides a compound wherein Ring E is a 5 membered partially unsaturated ring having 1-2 heteroatoms independently selected from nitrogen, oxygen, or sulfur, wherein Q is an optionally substituted C₁₋₁₀ alkylene chain wherein one, two, or three methylene units of Q are optionally and independently replaced by —O—, —N(R)—, —S—, or —Cy-, and wherein R¹⁰ of the Q-R¹⁰ moiety is selected from the group consisting of hydrogen, halogen, a suitably protected hydroxyl group, a suitably protected thiol group, or a suitably protected amino group.

In certain embodiments, the present invention provides a compound wherein Ring E is a 5 membered partially unsaturated ring having 1-2 heteroatoms independently selected from nitrogen, oxygen, or sulfur, wherein Q is an optionally substituted C₁₋₁₀ alkylene chain wherein one, two, or three methylene units of Q are optionally and independently replaced by —O—, —N(R)—, —S—, or —Cy-, and wherein R¹⁰ of the Q-R¹⁰ moiety is a ring optionally substituted at any substitutable carbon with 1-5 R¹¹ and at any substitutable nitrogen with R¹², wherein each R¹¹ is independently selected from halogen, R, OR, SR, N(R)₂, N(R)C(O)R, N(R)C(O)OR, N(R)C(O)N(R)₂, N(R)SO₂R, N(R)SO₂OR, S(O)R, SO₂R, OSO₂R, C(O)R, CO₂R, OCO₂R, C(O)N(R)₂, or OC(O)N(R)₂. In certain embodiments, the compound is as described above and R¹⁰ is an optionally substituted heterocycle. In certain embodiments, the compound is as described above and R¹⁰ is an optionally substituted 5-6 membered heterocycle with 1-3 heteroatoms. In certain embodiments, the compound is as described above and R¹⁰ is an optionally substituted 6 membered heterocycle with 2 heteroatoms. In certain embodiments, the compound is as described above and R¹⁰ is optionally substituted morpholine.

In certain embodiments, the present invention provides a compound wherein Ring E is a 5 membered partially unsaturated ring having 1-2 heteroatoms independently selected from nitrogen, oxygen, or sulfur, wherein Q is an optionally substituted C₁₋₁₀ alkylene chain wherein one, two, or three methylene units of Q are optionally and independently replaced by —O—, —N(R)—, —S—, or —Cy-, and wherein R¹⁰ of the Q-R¹⁰ moiety is a sugar-containing or sugar-like moiety.

In certain embodiments, the present invention provides a compound wherein Ring E is a 5 membered aromatic ring having 1-2 heteroatoms independently selected from nitrogen, oxygen, or sulfur, wherein Q is an optionally substituted C₁₋₁₀ alkylene chain wherein one, two, or three methylene units of Q are optionally and independently replaced by —O—, —N(R)—, —S—, or —Cy-, and wherein R¹⁰ of the Q-R¹⁰ moiety is selected from the group consisting of hydrogen, halogen, a suitably protected hydroxyl group, a suitably protected thiol group, or a suitably protected amino group.

In certain embodiments, the present invention provides a compound wherein Ring E is a 5 membered aromatic ring having 1-2 heteroatoms independently selected from nitrogen, oxygen, or sulfur, wherein Q is an optionally substituted C₁₋₁₀ alkylene chain wherein one, two, or three methylene units of Q are optionally and independently replaced by —O—, —N(R)—, —S—, or —Cy-, and wherein R¹⁰ of the Q-R¹⁰ moiety is a ring optionally substituted at any substitutable carbon with 1-5 R¹¹ and at any substitutable nitrogen with R¹², wherein each R¹¹ is independently selected from halogen, R, OR, SR, N(R)₂, N(R)C(O)R, N(R)C(O)OR, N(R)C(O)N(R)₂, N(R)SO₂R, N(R)SO₂OR, S(O)R, SO₂R, OSO₂R, C(O)R, CO₂R, OCO₂R, C(O)N(R)₂, or OC(O)N(R)₂. In certain embodiments, the compound is as described above and R¹⁰ is an optionally substituted heterocycle. In certain embodiments, the compound is as described above and R¹⁰ is an optionally substituted 5-6 membered heterocycle with 1-3 heteroatoms. In certain embodiments, the compound is as described above and R¹⁰ is an optionally substituted 6 membered heterocycle with 2 heteroatoms. In certain embodiments, the compound is as described above and R¹⁰ is optionally substituted morpholine.

In certain embodiments, the present invention provides a compound wherein Ring E is a 5 membered aromatic ring having 1-2 heteroatoms independently selected from nitrogen, oxygen, or sulfur, wherein Q is an optionally substituted C₁₋₁₀ alkylene chain wherein one, two, or three methylene units of Q are optionally and independently replaced by —O—, —N(R)—, —S—, or —Cy-, and wherein R¹⁰ of the Q-R¹⁰ moiety is a sugar-containing or sugar-like moiety.

In some embodiments, the present invention provides a compound wherein Ring E is a 6 membered saturated, partially unsaturated, or aromatic carbocycle, wherein Q is an optionally substituted C₁₋₁₀ alkylene chain wherein one, two, or three methylene units of Q are optionally and independently replaced by —O—, —N(R)—, —S—, —C(O)—, —OC(O)—, —C(O)O—, —OC(O)O—, —S(O)—, or —S(O)₂—, —OSO₂O—, —N(R)C(O)—, —C(O)NR—, —N(R)C(O)O—, —OC(O)NR—, —N(R)C(O)NR—, or —Cy-, wherein:

each —Cy- is independently a bivalent optionally substituted saturated, partially unsaturated, or aromatic monocyclic or bicyclic ring selected from a 6-10 membered arylene, a 5-10 membered heteroarylene having 1-4 heteroatoms independently selected from oxygen, nitrogen, or sulfur, a 3-8 membered carbocyclylene, or a 3-10 membered heterocyclylene having 1-4 heteroatoms independently selected from oxygen, nitrogen, or sulfur, and:

wherein R¹⁰ of the Q-R¹⁰ moiety is selected from the group consisting of hydrogen, halogen, an optionally substituted C₁₋₁₀ aliphatic, a suitably protected hydroxyl group, a suitably protected thiol group, a suitably protected amino group, an optionally substituted 3-8 membered saturated, partially unsaturated, or aryl monocyclic ring having 0-4 heteroatoms independently selected from nitrogen, oxygen, or sulfur, an optionally substituted 8-10 membered saturated, partially unsaturated, or aryl bicyclic ring having 0-4 heteroatoms independently selected from nitrogen, oxygen, or sulfur, a detectable moiety, a polymer residue, a peptide, a sugar-containing or sugar-like moiety, or:

wherein when R¹⁰ is a ring, R¹⁰ is optionally substituted at any substitutable carbon with 1-5 R¹¹ and at any substitutable nitrogen with R¹²;

each R¹¹ is independently halogen, R, OR, SR, N(R)₂, N(R)C(O)R, N(R)C(O)OR, N(R)C(O)N(R)₂, N(R)SO₂R, N(R)SO₂OR, S(O)R, SO₂R, OSO₂R, C(O)R, CO₂R, OCO₂R, C(O)N(R)₂, or OC(O)N(R)₂, or wherein:

two R¹¹ are optionally taken together to form an oxo moiety or an optionally substituted 3-8 membered saturated or partially unsaturated fused or spirofused ring having 0-4 heteroatoms independently selected from nitrogen, oxygen, or sulfur; and

each R¹² is independently R, OR, S(O)R, SO₂R, OSO₂R, C(O)R, CO₂R, OCO₂R, C(O)N(R)₂, or OC(O)N(R)₂, or wherein:

R¹² and R¹¹ are optionally taken together to form an optionally substituted 3-8 membered saturated or partially unsaturated fused ring having 0-4 heteroatoms independently selected from nitrogen, oxygen, or sulfur.

In certain embodiments, the present invention provides a compound wherein Ring E is a 6 membered saturated carbocycle, wherein Q is an optionally substituted C₁₋₁₀ alkylene chain wherein one, two, or three methylene units of Q are optionally and independently replaced by —O—, —N(R)—, —S—, or —Cy-, and wherein R¹⁰ of the Q-R¹⁰ moiety is selected from the group consisting of hydrogen, halogen, a suitably protected hydroxyl group, a suitably protected thiol group, or a suitably protected amino group.

In certain embodiments, the present invention provides a compound wherein Ring E is a 6 membered saturated carbocycle, wherein Q is an optionally substituted C₁₋₁₀ alkylene chain wherein one, two, or three methylene units of Q are optionally and independently replaced by —O—, —N(R)—, —S—, or —Cy-, and wherein R¹⁰ of the Q-R¹⁰ moiety is a ring optionally substituted at any substitutable carbon with 1-5 R¹¹ and at any substitutable nitrogen with R¹², wherein each R¹¹ is independently selected from halogen, R, OR, SR, N(R)₂, N(R)C(O)R, N(R)C(O)OR, N(R)C(O)N(R)₂, N(R)SO₂R, N(R)SO₂OR, S(O)R, SO₂R, OSO₂R, C(O)R, CO₂R, OCO₂R, C(O)N(R)₂, or OC(O)N(R)₂. In certain embodiments, the compound is as described above and R¹⁰ is an optionally substituted heterocycle. In certain embodiments, the compound is as described above and R¹⁰ is an optionally substituted 5-6 membered heterocycle with 1-3 heteroatoms. In certain embodiments, the compound is as described above and R¹⁰ is an optionally substituted 6 membered heterocycle with 2 heteroatoms. In certain embodiments, the compound is as described above and R¹⁰ is optionally substituted morpholine.

In certain embodiments, the present invention provides a compound wherein Ring E is a 6 membered saturated carbocycle, wherein Q is an optionally substituted C₁₋₁₀ alkylene chain wherein one, two, or three methylene units of Q are optionally and independently replaced by —O—, —N(R)—, —S—, or —Cy-, and wherein R¹⁰ of the Q-R¹⁰ moiety is a sugar-containing or sugar-like moiety.

In certain embodiments, the present invention provides a compound wherein Ring E is a 6 membered partially unsaturated carbocycle, wherein Q is an optionally substituted C₁₋₁₀ alkylene chain wherein one, two, or three methylene units of Q are optionally and independently replaced by —O—, —N(R)—, —S—, or —Cy-, and wherein R¹⁰ of the Q-R¹⁰ moiety is selected from the group consisting of hydrogen, halogen, a suitably protected hydroxyl group, a suitably protected thiol group, or a suitably protected amino group.

In certain embodiments, the present invention provides a compound wherein Ring E is a 6 membered partially unsaturated carbocycle, wherein Q is an optionally substituted C₁₋₁₀ alkylene chain wherein one, two, or three methylene units of Q are optionally and independently replaced by —O—, —N(R)—, —S—, or —Cy-, and wherein R¹⁰ of the Q-R¹⁰ moiety is a ring optionally substituted at any substitutable carbon with 1-5 R¹¹ and at any substitutable nitrogen with R¹², wherein each R¹¹ is independently selected from halogen, R, OR, SR, N(R)₂, N(R)C(O)R, N(R)C(O)OR, N(R)C(O)N(R)₂, N(R)SO₂R, N(R)SO₂OR, S(O)R, SO₂R, OSO₂R, C(O)R, CO₂R, OCO₂R, C(O)N(R)₂, or OC(O)N(R)₂. In certain embodiments, the compound is as described above and R¹⁰ is an optionally substituted heterocycle. In certain embodiments, the compound is as described above and R¹⁰ is an optionally substituted 5-6 membered heterocycle with 1-3 heteroatoms. In certain embodiments, the compound is as described above and R¹⁰ is an optionally substituted 6 membered heterocycle with 2 heteroatoms. In certain embodiments, the compound is as described above and R¹⁰ is optionally substituted morpholine.

In certain embodiments, the present invention provides a compound wherein Ring E is a 6 membered partially unsaturated carbocycle, wherein Q is an optionally substituted C₁₋₁₀ alkylene chain wherein one, two, or three methylene units of Q are optionally and independently replaced by —O—, —N(R)—, —S—, or —Cy-, and wherein R¹⁰ of the Q-R¹⁰ moiety is a sugar-containing or sugar-like moiety.

In certain embodiments, the present invention provides a compound wherein Ring E is a 6 membered aromatic carbocycle, wherein Q is an optionally substituted C₁₋₁₀ alkylene chain wherein one, two, or three methylene units of Q are optionally and independently replaced by —O—, —N(R)—, —S—, or —Cy-, and wherein R¹⁰ of the Q-R¹⁰ moiety is selected from the group consisting of hydrogen, halogen, a suitably protected hydroxyl group, a suitably protected thiol group, or a suitably protected amino group.

In certain embodiments, the present invention provides a compound wherein Ring E is a 6 membered aromatic carbocycle, wherein Q is an optionally substituted C₁₋₁₀ alkylene chain wherein one, two, or three methylene units of Q are optionally and independently replaced by —O—, —N(R)—, —S—, or —Cy-, and wherein R¹⁰ of the Q-R¹⁰ moiety is a ring optionally substituted at any substitutable carbon with 1-5 R¹¹ and at any substitutable nitrogen with R¹², wherein each R¹¹ is independently selected from halogen, R, OR, SR, N(R)₂, N(R)C(O)R, N(R)C(O)OR, N(R)C(O)N(R)₂, N(R)SO₂R, N(R)SO₂OR, S(O)R, SO₂R, OSO₂R, C(O)R, CO₂R, OCO₂R, C(O)N(R)₂, or OC(O)N(R)₂. In certain embodiments, the compound is as described above and R¹⁰ is an optionally substituted heterocycle. In certain embodiments, the compound is as described above and R¹⁰ is an optionally substituted 5-6 membered heterocycle with 1-3 heteroatoms. In certain embodiments, the compound is as described above and R¹⁰ is an optionally substituted 6 membered heterocycle with 2 heteroatoms. In certain embodiments, the compound is as described above and R¹⁰ is optionally substituted morpholine.

In certain embodiments, the present invention provides a compound wherein Ring E is a 6 membered aromatic carbocycle, wherein Q is an optionally substituted C₁₋₁₀ alkylene chain wherein one, two, or three methylene units of Q are optionally and independently replaced by —O—, —N(R)—, —S—, or —Cy-, and wherein R¹⁰ of the Q-R¹⁰ moiety is a sugar-containing or sugar-like moiety.

In some embodiments, the present invention provides a compound wherein Ring E is a 6 membered saturated, partially unsaturated, or aromatic ring having 1-2 heteroatoms independently selected from nitrogen, oxygen, or sulfur, and wherein Q is an optionally substituted C₁₋₁₀ alkylene chain wherein one, two, or three methylene units of Q are optionally and independently replaced by —O—, —N(R)—, —S—, —C(O)—, —OC(O)—, —C(O)O—, —OC(O)O—, —S(O)—, or —S(O)₂—, —OSO₂O—, —N(R)C(O)—, —C(O)NR—, —N(R)C(O)O—, —OC(O)NR—, —N(R)C(O)NR—, or —Cy-, wherein:

each —Cy- is independently a bivalent optionally substituted saturated, partially unsaturated, or aromatic monocyclic or bicyclic ring selected from a 6-10 membered arylene, a 5-10 membered heteroarylene having 1-4 heteroatoms independently selected from oxygen, nitrogen, or sulfur, a 3-8 membered carbocyclylene, or a 3-10 membered heterocyclylene having 1-4 heteroatoms independently selected from oxygen, nitrogen, or sulfur, and:

wherein R¹⁰ of the Q-R¹⁰ moiety is selected from the group consisting of hydrogen, halogen, an optionally substituted C₁₋₁₀ aliphatic, a suitably protected hydroxyl group, a suitably protected thiol group, a suitably protected amino group, an optionally substituted 3-8 membered saturated, partially unsaturated, or aryl monocyclic ring having 0-4 heteroatoms independently selected from nitrogen, oxygen, or sulfur, an optionally substituted 8-10 membered saturated, partially unsaturated, or aryl bicyclic ring having 0-4 heteroatoms independently selected from nitrogen, oxygen, or sulfur, a detectable moiety, a polymer residue, a peptide, a sugar-containing or sugar-like moiety, or:

wherein when R¹⁰ is a ring, R¹⁰ is optionally substituted at any substitutable carbon with 1-5 R¹¹ and at any substitutable nitrogen with R¹²;

each R¹¹ is independently halogen, R, OR, SR, N(R)₂, N(R)C(O)R, N(R)C(O)OR, N(R)C(O)N(R)₂, N(R)SO₂R, N(R)SO₂OR, S(O)R, SO₂R, OSO₂R, C(O)R, CO₂R, OCO₂R, C(O)N(R)₂, or OC(O)N(R)₂, or wherein:

two R¹¹ are optionally taken together to form an oxo moiety or an optionally substituted 3-8 membered saturated or partially unsaturated fused or spirofused ring having 0-4 heteroatoms independently selected from nitrogen, oxygen, or sulfur; and

each R¹² is independently R, OR, S(O)R, SO₂R, OSO₂R, C(O)R, CO₂R, OCO₂R, C(O)N(R)₂, or OC(O)N(R)₂, or wherein:

R¹² and R¹¹ are optionally taken together to form an optionally substituted 3-8 membered saturated or partially unsaturated fused ring having 0-4 heteroatoms independently selected from nitrogen, oxygen, or sulfur.

In certain embodiments, the present invention provides a compound wherein Ring E is a 6 membered saturated ring having 1-2 heteroatoms independently selected from nitrogen, oxygen, or sulfur, wherein Q is an optionally substituted C₁₋₁₀ alkylene chain wherein one, two, or three methylene units of Q are optionally and independently replaced by —O—, —N(R)—, —S—, or —Cy-, and wherein R¹⁰ of the Q-R¹⁰ moiety is selected from the group consisting of hydrogen, halogen, a suitably protected hydroxyl group, a suitably protected thiol group, or a suitably protected amino group.

In certain embodiments, the present invention provides a compound wherein Ring E is a 6 membered saturated ring having 1-2 heteroatoms independently selected from nitrogen, oxygen, or sulfur, wherein Q is an optionally substituted C₁₋₁₀ alkylene chain wherein one, two, or three methylene units of Q are optionally and independently replaced by —O—, —N(R)—, —S—, or —Cy-, and wherein R¹⁰ of the Q-R¹⁰ moiety is a ring optionally substituted at any substitutable carbon with 1-5 R¹¹ and at any substitutable nitrogen with R¹², wherein each R¹¹ is independently selected from halogen, R, OR, SR, N(R)₂, N(R)C(O)R, N(R)C(O)OR, N(R)C(O)N(R)₂, N(R)SO₂R, N(R)SO₂OR, S(O)R, SO₂R, OSO₂R, C(O)R, CO₂R, OCO₂R, C(O)N(R)₂, or OC(O)N(R)₂. In certain embodiments, the compound is as described above and R¹⁰ is an optionally substituted heterocycle. In certain embodiments, the compound is as described above and R¹⁰ is an optionally substituted 5-6 membered heterocycle with 1-3 heteroatoms. In certain embodiments, the compound is as described above and R¹⁰ is an optionally substituted 6 membered heterocycle with 2 heteroatoms. In certain embodiments, the compound is as described above and R¹⁰ is optionally substituted morpholine.

In certain embodiments, the present invention provides a compound wherein Ring E is a 6 membered saturated ring having 1-2 heteroatoms independently selected from nitrogen, oxygen, or sulfur, wherein Q is an optionally substituted C₁₋₁₀ alkylene chain wherein one, two, or three methylene units of Q are optionally and independently replaced by —O—, —N(R)—, —S—, or —Cy-, and wherein R¹⁰ of the Q-R¹⁰ moiety is a sugar-containing or sugar-like moiety.

In certain embodiments, the present invention provides a compound wherein Ring E is a 6 membered partially unsaturated ring having 1-2 heteroatoms independently selected from nitrogen, oxygen, or sulfur, wherein Q is an optionally substituted C₁₋₁₀ alkylene chain wherein one, two, or three methylene units of Q are optionally and independently replaced by —O—, —N(R)—, —S—, or —Cy-, and wherein R¹⁰ of the Q-R¹⁰ moiety is selected from the group consisting of hydrogen, halogen, a suitably protected hydroxyl group, a suitably protected thiol group, or a suitably protected amino group.

In certain embodiments, the present invention provides a compound wherein Ring E is a 6 membered partially unsaturated ring having 1-2 heteroatoms independently selected from nitrogen, oxygen, or sulfur, wherein Q is an optionally substituted C₁₋₁₀ alkylene chain wherein one, two, or three methylene units of Q are optionally and independently replaced by —O—, —N(R)—, —S—, or —Cy-, and wherein R¹⁰ of the Q-R¹⁰ moiety is a ring optionally substituted at any substitutable carbon with 1-5 R¹¹ and at any substitutable nitrogen with R¹², wherein each R¹¹ is independently selected from halogen, R, OR, SR, N(R)₂, N(R)C(O)R, N(R)C(O)OR, N(R)C(O)N(R)₂, N(R)SO₂R, N(R)SO₂OR, S(O)R, SO₂R, OSO₂R, C(O)R, CO₂R, OCO₂R, C(O)N(R)₂, or OC(O)N(R)₂. In certain embodiments, the compound is as described above and R¹⁰ is an optionally substituted heterocycle. In certain embodiments, the compound is as described above and R¹⁰ is an optionally substituted 5-6 membered heterocycle with 1-3 heteroatoms. In certain embodiments, the compound is as described above and R¹⁰ is an optionally substituted 6 membered heterocycle with 2 heteroatoms. In certain embodiments, the compound is as described above and R¹⁰ is optionally substituted morpholine.

In certain embodiments, the present invention provides a compound wherein Ring E is a 6 membered partially unsaturated ring having 1-2 heteroatoms independently selected from nitrogen, oxygen, or sulfur, wherein Q is an optionally substituted C₁₋₁₀ alkylene chain wherein one, two, or three methylene units of Q are optionally and independently replaced by —O—, —N(R)—, —S—, or —Cy-, and wherein R¹⁰ of the Q-R¹⁰ moiety is a sugar-containing or sugar-like moiety.

In certain embodiments, the present invention provides a compound wherein Ring E is a 6 membered aromatic ring having 1-2 heteroatoms independently selected from nitrogen, oxygen, or sulfur, wherein Q is an optionally substituted C₁₋₁₀ alkylene chain wherein one, two, or three methylene units of Q are optionally and independently replaced by —O—, —N(R)—, —S—, or —Cy-, and wherein R¹⁰ of the Q-R¹⁰ moiety is selected from the group consisting of hydrogen, halogen, a suitably protected hydroxyl group, a suitably protected thiol group, or a suitably protected amino group.

In certain embodiments, the present invention provides a compound wherein Ring E is a 6 membered aromatic ring having 1-2 heteroatoms independently selected from nitrogen, oxygen, or sulfur, wherein Q is an optionally substituted C₁₋₁₀ alkylene chain wherein one, two, or three methylene units of Q are optionally and independently replaced by —O—, —N(R)—, —S—, or —Cy-, and wherein R¹⁰ of the Q-R¹⁰ moiety is a ring optionally substituted at any substitutable carbon with 1-5 R¹¹ and at any substitutable nitrogen with R¹², wherein each R¹¹ is independently selected from halogen, R, OR, SR, N(R)₂, N(R)C(O)R, N(R)C(O)OR, N(R)C(O)N(R)₂, N(R)SO₂R, N(R)SO₂OR, S(O)R, SO₂R, OSO₂R, C(O)R, CO₂R, OCO₂R, C(O)N(R)₂, or OC(O)N(R)₂. In certain embodiments, the compound is as described above and R¹⁰ is an optionally substituted heterocycle. In certain embodiments, the compound is as described above and R¹⁰ is an optionally substituted 5-6 membered heterocycle with 1-3 heteroatoms. In certain embodiments, the compound is as described above and R¹⁰ is an optionally substituted 6 membered heterocycle with 2 heteroatoms. In certain embodiments, the compound is as described above and R¹⁰ is optionally substituted morpholine.

In certain embodiments, the present invention provides a compound wherein Ring E is a 6 membered aromatic ring having 1-2 heteroatoms independently selected from nitrogen, oxygen, or sulfur, wherein Q is an optionally substituted C₁₋₁₀ alkylene chain wherein one, two, or three methylene units of Q are optionally and independently replaced by —O—, —N(R)—, —S—, or —Cy-, and wherein R¹⁰ of the Q-R¹⁰ moiety is a sugar-containing or sugar-like moiety.

In some embodiments, the present invention provides a compound wherein Ring E is a 7 membered saturated or partially unsaturated carbocycle, wherein Q is an optionally substituted C₁₋₁₀ alkylene chain wherein one, two, or three methylene units of Q are optionally and independently replaced by —O—, —N(R)—, —S—, —C(O)—, —OC(O)—, —C(O)O—, —OC(O)O—, —S(O)—, or —S(O)₂—, —OSO₂O—, —N(R)C(O)—, —C(O)NR—, —N(R)C(O)O—, —OC(O)NR—, —N(R)C(O)NR—, or —Cy-, wherein:

each —Cy- is independently a bivalent optionally substituted saturated, partially unsaturated, or aromatic monocyclic or bicyclic ring selected from a 6-10 membered arylene, a 5-10 membered heteroarylene having 1-4 heteroatoms independently selected from oxygen, nitrogen, or sulfur, a 3-8 membered carbocyclylene, or a 3-10 membered heterocyclylene having 1-4 heteroatoms independently selected from oxygen, nitrogen, or sulfur, and:

wherein R¹⁰ of the Q-R¹⁰ moiety is selected from the group consisting of hydrogen, halogen, an optionally substituted C₁₋₁₀ aliphatic, a suitably protected hydroxyl group, a suitably protected thiol group, a suitably protected amino group, an optionally substituted 3-8 membered saturated, partially unsaturated, or aryl monocyclic ring having 0-4 heteroatoms independently selected from nitrogen, oxygen, or sulfur, an optionally substituted 8-10 membered saturated, partially unsaturated, or aryl bicyclic ring having 0-4 heteroatoms independently selected from nitrogen, oxygen, or sulfur, a detectable moiety, a polymer residue, a peptide, a sugar-containing or sugar-like moiety, or:

wherein when R¹⁰ is a ring, R¹⁰ is optionally substituted at any substitutable carbon with 1-5 R¹¹ and at any substitutable nitrogen with R¹²;

each R¹¹ is independently halogen, R, OR, SR, N(R)₂, N(R)C(O)R, N(R)C(O)OR, N(R)C(O)N(R)₂, N(R)SO₂R, N(R)SO₂OR, S(O)R, SO₂R, OSO₂R, C(O)R, CO₂R, OCO₂R, C(O)N(R)₂, or OC(O)N(R)₂, or wherein:

two R¹¹ are optionally taken together to form an oxo moiety or an optionally substituted 3-8 membered saturated or partially unsaturated fused or spirofused ring having 0-4 heteroatoms independently selected from nitrogen, oxygen, or sulfur; and

each R¹² is independently R, OR, S(O)R, SO₂R, OSO₂R, C(O)R, CO₂R, OCO₂R, C(O)N(R)₂, or OC(O)N(R)₂, or wherein:

R¹² and R¹¹ are optionally taken together to form an optionally substituted 3-8 membered saturated or partially unsaturated fused ring having 0-4 heteroatoms independently selected from nitrogen, oxygen, or sulfur.

In certain embodiments, the present invention provides a compound wherein Ring E is a 7 membered saturated carbocycle, wherein Q is an optionally substituted C₁₋₁₀ alkylene chain wherein one, two, or three methylene units of Q are optionally and independently replaced by —O—, —N(R)—, —S—, or —Cy-, and wherein R¹⁰ of the Q-R¹⁰ moiety is selected from the group consisting of hydrogen, halogen, a suitably protected hydroxyl group, a suitably protected thiol group, or a suitably protected amino group.

In certain embodiments, the present invention provides a compound wherein Ring E is a 7 membered saturated carbocycle, wherein Q is an optionally substituted C₁₋₁₀ alkylene chain wherein one, two, or three methylene units of Q are optionally and independently replaced by —O—, —N(R)—, —S—, or —Cy-, and wherein R¹⁰ of the Q-R¹⁰ moiety is a ring optionally substituted at any substitutable carbon with 1-5 R¹¹ and at any substitutable nitrogen with R¹², wherein each R¹¹ is independently selected from halogen, R, OR, SR, N(R)₂, N(R)C(O)R, N(R)C(O)OR, N(R)C(O)N(R)₂, N(R)SO₂R, N(R)SO₂OR, S(O)R, SO₂R, OSO₂R, C(O)R, CO₂R, OCO₂R, C(O)N(R)₂, or OC(O)N(R)₂. In certain embodiments, the compound is as described above and R¹⁰ is an optionally substituted heterocycle. In certain embodiments, the compound is as described above and R¹⁰ is an optionally substituted 5-6 membered heterocycle with 1-3 heteroatoms. In certain embodiments, the compound is as described above and R¹⁰ is an optionally substituted 6 membered heterocycle with 2 heteroatoms. In certain embodiments, the compound is as described above and R¹⁰ is optionally substituted morpholine.

In certain embodiments, the present invention provides a compound wherein Ring E is a 7 membered saturated carbocycle, wherein Q is an optionally substituted C₁₋₁₀ alkylene chain wherein one, two, or three methylene units of Q are optionally and independently replaced by —O—, —N(R)—, —S—, or —Cy-, and wherein R¹⁰ of the Q-R¹⁰ moiety is a sugar-containing or sugar-like moiety.

In certain embodiments, the present invention provides a compound wherein Ring E is a 7 membered partially unsaturated carbocycle, wherein Q is an optionally substituted C₁₋₁₀ alkylene chain wherein one, two, or three methylene units of Q are optionally and independently replaced by —O—, —N(R)—, —S—, or —Cy-, and wherein R¹⁰ of the Q-R¹⁰ moiety is selected from the group consisting of hydrogen, halogen, a suitably protected hydroxyl group, a suitably protected thiol group, or a suitably protected amino group.

In certain embodiments, the present invention provides a compound wherein Ring E is a 7 membered partially unsaturated carbocycle, wherein Q is an optionally substituted C₁₋₁₀ alkylene chain wherein one, two, or three methylene units of Q are optionally and independently replaced by —O—, —N(R)—, —S—, or —Cy-, and wherein R¹⁰ of the Q-R¹⁰ moiety is a ring optionally substituted at any substitutable carbon with 1-5 R¹¹ and at any substitutable nitrogen with R¹², wherein each R¹¹ is independently selected from halogen, R, OR, SR, N(R)₂, N(R)C(O)R, N(R)C(O)OR, N(R)C(O)N(R)₂, N(R)SO₂R, N(R)SO₂OR, S(O)R, SO₂R, OSO₂R, C(O)R, CO₂R, OCO₂R, C(O)N(R)₂, or OC(O)N(R)₂. In certain embodiments, the compound is as described above and R¹⁰ is an optionally substituted heterocycle. In certain embodiments, the compound is as described above and R¹⁰ is an optionally substituted 5-6 membered heterocycle with 1-3 heteroatoms. In certain embodiments, the compound is as described above and R¹⁰ is an optionally substituted 6 membered heterocycle with 2 heteroatoms. In certain embodiments, the compound is as described above and R¹⁰ is optionally substituted morpholine.

In certain embodiments, the present invention provides a compound wherein Ring E is a 7 membered partially unsaturated carbocycle, wherein Q is an optionally substituted C₁₋₁₀ alkylene chain wherein one, two, or three methylene units of Q are optionally and independently replaced by —O—, —N(R)—, —S—, or —Cy-, and wherein R¹⁰ of the Q-R¹⁰ moiety is a sugar-containing or sugar-like moiety.

In some embodiments, the present invention provides a compound wherein Ring E is a 7 membered saturated or partially unsaturated ring having 1-2 heteroatoms independently selected from nitrogen, oxygen, or sulfur, and wherein Q are optionally and independently replaced by —O—, —N(R)—, —S—, —C(O)—, —OC(O)—, —C(O)O—, —OC(O)O—, —S(O)—, or —S(O)₂—, —OSO₂O—, —N(R)C(O)—, —C(O)NR—, —N(R)C(O)O—, —OC(O)NR—, —N(R)C(O)NR—, or —Cy-, wherein:

each —Cy- is independently a bivalent optionally substituted saturated, partially unsaturated, or aromatic monocyclic or bicyclic ring selected from a 6-10 membered arylene, a 5-10 membered heteroarylene having 1-4 heteroatoms independently selected from oxygen, nitrogen, or sulfur, a 3-8 membered carbocyclylene, or a 3-10 membered heterocyclylene having 1-4 heteroatoms independently selected from oxygen, nitrogen, or sulfur, and:

wherein R¹⁰ of the Q-R¹⁰ moiety is selected from the group consisting of hydrogen, halogen, an optionally substituted C₁₋₁₀ aliphatic, a suitably protected hydroxyl group, a suitably protected thiol group, a suitably protected amino group, an optionally substituted 3-8 membered saturated, partially unsaturated, or aryl monocyclic ring having 0-4 heteroatoms independently selected from nitrogen, oxygen, or sulfur, an optionally substituted 8-10 membered saturated, partially unsaturated, or aryl bicyclic ring having 0-4 heteroatoms independently selected from nitrogen, oxygen, or sulfur, a detectable moiety, a polymer residue, a peptide, a sugar-containing or sugar-like moiety, or:

wherein when R¹⁰ is a ring, R¹⁰ is optionally substituted at any substitutable carbon with 1-5 R¹¹ and at any substitutable nitrogen with R¹²;

each R¹¹ is independently halogen, R, OR, SR, N(R)₂, N(R)C(O)R, N(R)C(O)OR, N(R)C(O)N(R)₂, N(R)SO₂R, N(R)SO₂OR, S(O)R, SO₂R, OSO₂R, C(O)R, CO₂R, OCO₂R, C(O)N(R)₂, or OC(O)N(R)₂, or wherein:

two R¹¹ are optionally taken together to form an oxo moiety or an optionally substituted 3-8 membered saturated or partially unsaturated fused or spirofused ring having 0-4 heteroatoms independently selected from nitrogen, oxygen, or sulfur; and

each R¹² is independently R, OR, S(O)R, SO₂R, OSO₂R, C(O)R, CO₂R, OCO₂R, C(O)N(R)₂, or OC(O)N(R)₂, or wherein:

R¹² and R¹¹ are optionally taken together to form an optionally substituted 3-8 membered saturated or partially unsaturated fused ring having 0-4 heteroatoms independently selected from nitrogen, oxygen, or sulfur.

In certain embodiments, the present invention provides a compound wherein Ring E is a 7 membered saturated ring having 1-2 heteroatoms independently selected from nitrogen, oxygen, or sulfur, wherein Q is an optionally substituted C₁₋₁₀ alkylene chain wherein one, two, or three methylene units of Q are optionally and independently replaced by —O—, —N(R)—, —S—, or —Cy-, and wherein R¹⁰ of the Q-R¹⁰ moiety is selected from the group consisting of hydrogen, halogen, a suitably protected hydroxyl group, a suitably protected thiol group, or a suitably protected amino group.

In certain embodiments, the present invention provides a compound wherein Ring E is a 7 membered saturated ring having 1-2 heteroatoms independently selected from nitrogen, oxygen, or sulfur, wherein Q is an optionally substituted C₁₋₁₀ alkylene chain wherein one, two, or three methylene units of Q are optionally and independently replaced by —O—, —N(R)—, —S—, or —Cy-, and wherein R¹⁰ of the Q-R¹⁰ moiety is a ring optionally substituted at any substitutable carbon with 1-5 R¹¹ and at any substitutable nitrogen with R¹², wherein each R¹¹ is independently selected from halogen, R, OR, SR, N(R)₂, N(R)C(O)R, N(R)C(O)OR, N(R)C(O)N(R)₂, N(R)SO₂R, N(R)SO₂OR, S(O)R, SO₂R, OSO₂R, C(O)R, CO₂R, OCO₂R, C(O)N(R)₂, or OC(O)N(R)₂. In certain embodiments, the compound is as described above and R¹⁰ is an optionally substituted heterocycle. In certain embodiments, the compound is as described above and R¹⁰ is an optionally substituted 5-6 membered heterocycle with 1-3 heteroatoms. In certain embodiments, the compound is as described above and R¹⁰ is an optionally substituted 6 membered heterocycle with 2 heteroatoms. In certain embodiments, the compound is as described above and R¹⁰ is optionally substituted morpholine.

In certain embodiments, the present invention provides a compound wherein Ring E is a 7 membered saturated ring having 1-2 heteroatoms independently selected from nitrogen, oxygen, or sulfur, wherein Q is an optionally substituted C₁₋₁₀ alkylene chain wherein one, two, or three methylene units of Q are optionally and independently replaced by —O—, —N(R)—, —S—, or —Cy-, and wherein R¹⁰ of the Q-R¹⁰ moiety is a sugar-containing or sugar-like moiety.

In certain embodiments, the present invention provides a compound wherein Ring E is a 7 membered partially unsaturated ring having 1-2 heteroatoms independently selected from nitrogen, oxygen, or sulfur, wherein Q is an optionally substituted C₁₋₁₀ alkylene chain wherein one, two, or three methylene units of Q are optionally and independently replaced by —O—, —N(R)—, —S—, or —Cy-, and wherein R¹⁰ of the Q-R¹⁰ moiety is selected from the group consisting of hydrogen, halogen, a suitably protected hydroxyl group, a suitably protected thiol group, or a suitably protected amino group.

In certain embodiments, the present invention provides a compound wherein Ring E is a 7 membered partially unsaturated ring having 1-2 heteroatoms independently selected from nitrogen, oxygen, or sulfur, wherein Q is an optionally substituted C₁₋₁₀ alkylene chain wherein one, two, or three methylene units of Q are optionally and independently replaced by —O—, —N(R)—, —S—, or —Cy-, and wherein R¹⁰ of the Q-R¹⁰ moiety is a ring optionally substituted at any substitutable carbon with 1-5 R¹¹ and at any substitutable nitrogen with R¹², wherein each R¹¹ is independently selected from halogen, R, OR, SR, N(R)₂, N(R)C(O)R, N(R)C(O)OR, N(R)C(O)N(R)₂, N(R)SO₂R, N(R)SO₂OR, S(O)R, SO₂R, OSO₂R, C(O)R, CO₂R, OCO₂R, C(O)N(R)₂, or OC(O)N(R)₂. In certain embodiments, the compound is as described above and R¹⁰ is an optionally substituted heterocycle. In certain embodiments, the compound is as described above and R¹⁰ is an optionally substituted 5-6 membered heterocycle with 1-3 heteroatoms. In certain embodiments, the compound is as described above and R¹⁰ is an optionally substituted 6 membered heterocycle with 2 heteroatoms. In certain embodiments, the compound is as described above and R¹⁰ is optionally substituted morpholine.

In certain embodiments, the present invention provides a compound wherein Ring E is a 7 membered partially unsaturated ring having 1-2 heteroatoms independently selected from nitrogen, oxygen, or sulfur, wherein Q is an optionally substituted C₁₋₁₀ alkylene chain wherein one, two, or three methylene units of Q are optionally and independently replaced by —O—, —N(R)—, —S—, or —Cy-, and wherein R¹⁰ of the Q-R¹⁰ moiety is a sugar-containing or sugar-like moiety.

In certain embodiments, the present invention provides a compound wherein Ring E is a 7 membered aromatic ring having 1-2 heteroatoms independently selected from nitrogen, oxygen, or sulfur, wherein Q is an optionally substituted C₁₋₁₀ alkylene chain wherein one, two, or three methylene units of Q are optionally and independently replaced by —O—, —N(R)—, —S—, or —Cy-, and wherein R¹⁰ of the Q-R¹⁰ moiety is selected from the group consisting of hydrogen, halogen, a suitably protected hydroxyl group, a suitably protected thiol group, or a suitably protected amino group.

In certain embodiments, the present invention provides a compound wherein Ring E is a 7 membered aromatic ring having 1-2 heteroatoms independently selected from nitrogen, oxygen, or sulfur, wherein Q is an optionally substituted C₁₋₁₀ alkylene chain wherein one, two, or three methylene units of Q are optionally and independently replaced by —O—, —N(R)—, —S—, or —Cy-, and wherein R¹⁰ of the Q-R¹⁰ moiety is a ring optionally substituted at any substitutable carbon with 1-5 R¹¹ and at any substitutable nitrogen with R¹², wherein each R¹¹ is independently selected from halogen, R, OR, SR, N(R)₂, N(R)C(O)R, N(R)C(O)OR, N(R)C(O)N(R)₂, N(R)SO₂R, N(R)SO₂OR, S(O)R, SO₂R, OSO₂R, C(O)R, CO₂R, OCO₂R, C(O)N(R)₂, or OC(O)N(R)₂. In certain embodiments, the compound is as described above and R¹⁰ is an optionally substituted heterocycle. In certain embodiments, the compound is as described above and R¹⁰ is an optionally substituted 5-6 membered heterocycle with 1-3 heteroatoms. In certain embodiments, the compound is as described above and R¹⁰ is an optionally substituted 6 membered heterocycle with 2 heteroatoms. In certain embodiments, the compound is as described above and R¹⁰ is optionally substituted morpholine.

In certain embodiments, the present invention provides a compound wherein Ring E is a 7 membered aromatic ring having 1-2 heteroatoms independently selected from nitrogen, oxygen, or sulfur, wherein Q is an optionally substituted C₁₋₁₀ alkylene chain wherein one, two, or three methylene units of Q are optionally and independently replaced by —O—, —N(R)—, —S—, or —Cy-, and wherein R¹⁰ of the Q-R¹⁰ moiety is a sugar-containing or sugar-like moiety.

### Exemplary R⁵/Q-R¹⁰ Combinations

In some embodiments, the present invention provides a compound wherein each R⁵ of Ring E is independently T-C(R′)₃, T-C(R′)₂C(R″)₃, OR, a suitably protected hydroxyl group, SR, a suitably protected thiol group, S(O)R, SO₂R, OSO₂R, N(R)₂, a suitably protected amino group, N(R)C(O)R, N(R)C(O)C(O)R, N(R)C(O)N(R)₂, N(R)C(O)OR, C(O)OR, OC(O)R, C(O)N(R)₂, or OC(O)N(R)₂, an optionally substituted 3-8 membered saturated, partially unsaturated, or aryl monocyclic ring having 0-4 heteroatoms independently selected from nitrogen, oxygen, or sulfur, an optionally substituted 8-10 membered saturated, partially unsaturated, or aryl bicyclic ring having 0-4 heteroatoms independently selected from nitrogen, oxygen, or sulfur;

each T is independently a valence bond or an optionally substituted straight or branched, saturated or unsaturated, C₁₋₆ alkylene chain wherein up to two methylene units of T are optionally and independently replaced by —O—, —N(R)—, —S—, —C(O)—, —S(O)—, or —S(O)₂—;

each R′ and R″ is independently selected from halogen, R, OR, SR, S(O)R, SO₂R, OSO₂R, N(R)₂, N(R)C(O)R, N(R)C(O)C(O)R, N(R)C(O)N(R)₂, N(R)C(O)OR, N(R)S(O)R, N(R)SO₂R, N(R)SO₂OR C(O)OR, OC(O)R, C(O)N(R)₂, OC(O)N(R)₂, or an optionally substituted 3-8 membered saturated, partially unsaturated, or aryl monocyclic ring having 0-4 heteroatoms independently selected from nitrogen, oxygen, or sulfur, or an optionally substituted 8-10 membered saturated, partially unsaturated, or aryl bicyclic ring having 0-4 heteroatoms independently selected from nitrogen, oxygen, or sulfur, or:

two R′ are optionally taken together to form an oxo moiety, an oxime, an optionally substituted hydrazone, an optionally substituted imine, or an optionally substituted 3-8 membered saturated or partially unsaturated ring having 0-4 heteroatoms independently selected from nitrogen, oxygen, or sulfur, or:

two R″ are optionally taken together to form an oxo moiety, an oxime, an optionally substituted hydrazone, an optionally substituted imine, or an optionally substituted 3-8 membered saturated or partially unsaturated ring having 0-4 heteroatoms independently selected from nitrogen, oxygen, or sulfur; and

wherein Q is a valence bond and wherein R¹⁰ of the Q-R¹⁰ moiety is selected from the group consisting of hydrogen, halogen, an optionally substituted C₁₋₁₀ aliphatic, a suitably protected hydroxyl group, a suitably protected thiol group, a suitably protected amino group, an optionally substituted 3-8 membered saturated, partially unsaturated, or aryl monocyclic ring having 0-4 heteroatoms independently selected from nitrogen, oxygen, or sulfur, an optionally substituted 8-10 membered saturated, partially unsaturated, or aryl bicyclic ring having 0-4 heteroatoms independently selected from nitrogen, oxygen, or sulfur, a detectable moiety, a polymer residue, a peptide, a sugar-containing or sugar-like moiety, or:

wherein when R¹⁰ is a ring, R¹⁰ is optionally substituted at any substitutable carbon with 1-5 R¹¹ and at any substitutable nitrogen with R¹²;

each R¹¹ is independently halogen, R, OR, SR, N(R)₂, N(R)C(O)R, N(R)C(O)OR, N(R)C(O)N(R)₂, N(R)SO₂R, N(R)SO₂OR, S(O)R, SO₂R, OSO₂R, C(O)R, CO₂R, OCO₂R, C(O)N(R)₂, or OC(O)N(R)₂, or wherein:

two R¹¹ are optionally taken together to form an oxo moiety or an optionally substituted 3-8 membered saturated or partially unsaturated fused or spirofused ring having 0-4 heteroatoms independently selected from nitrogen, oxygen, or sulfur; and

each R¹² is independently R, OR, S(O)R, SO₂R, OSO₂R, C(O)R, CO₂R, OCO₂R, C(O)N(R)₂, or OC(O)N(R)₂, or wherein:

R¹² and R¹¹ are optionally taken together to form an optionally substituted 3-8 membered saturated or partially unsaturated fused ring having 0-4 heteroatoms independently selected from nitrogen, oxygen, or sulfur.

In certain embodiments, the present invention provides a compound wherein each R⁵ of Ring E is independently T-C(R′)₃, T-C(R′)₂C(R″)₃, OR, or a suitably protected hydroxyl group, wherein Q is a valence bond and wherein R¹⁰ of the Q-R¹⁰ moiety is selected from the group consisting of hydrogen, halogen, a suitably protected hydroxyl group, a suitably protected thiol group, or a suitably protected amino group.

In certain embodiments, the present invention provides a compound wherein each R⁵ of Ring E is independently T-C(R′)₃, T-C(R′)₂C(R″)₃, OR, or a suitably protected hydroxyl group, wherein Q is a valence bond, and wherein R¹⁰ of the Q-R¹⁰ moiety is a ring optionally substituted at any substitutable carbon with 1-5 R¹¹ and at any substitutable nitrogen with R¹², wherein each R¹¹ is independently selected from halogen, R, OR, SR, N(R)₂, N(R)C(O)R, N(R)C(O)OR, N(R)C(O)N(R)₂, N(R)SO₂R, N(R)SO₂OR, S(O)R, SO₂R, OSO₂R, C(O)R, CO₂R, OCO₂R, C(O)N(R)₂, or OC(O)N(R)₂.

In certain embodiments, the present invention provides a compound wherein each R⁵ of Ring E is independently T-C(R′)₃, T-C(R′)₂C(R″)₃, OR, or a suitably protected hydroxyl group, wherein Q is a valence bond, and wherein R¹⁰ of the Q-R¹⁰ moiety is a sugar-containing or sugar-like moiety.

In some embodiments, the present invention provides a compound wherein each R⁵ of Ring E is independently T-C(R′)₃, T-C(R′)₂C(R″)₃, OR, a suitably protected hydroxyl group, SR, a suitably protected thiol group, S(O)R, SO₂R, OSO₂R, N(R)₂, a suitably protected amino group, N(R)C(O)R, N(R)C(O)C(O)R, N(R)C(O)N(R)₂, N(R)C(O)OR, C(O)OR, OC(O)R, C(O)N(R)₂, or OC(O)N(R)₂, an optionally substituted 3-8 membered saturated, partially unsaturated, or aryl monocyclic ring having 0-4 heteroatoms independently selected from nitrogen, oxygen, or sulfur, an optionally substituted 8-10 membered saturated, partially unsaturated, or aryl bicyclic ring having 0-4 heteroatoms independently selected from nitrogen, oxygen, or sulfur;

each T is independently a valence bond or an optionally substituted straight or branched, saturated or unsaturated, C₁₋₆ alkylene chain wherein up to two methylene units of T are optionally and independently replaced by —O—, —N(R)—, —S—, —C(O)—, —S(O)—, or —S(O)₂—;

each R′ and R″ is independently selected from halogen, R, OR, SR, S(O)R, SO₂R, OSO₂R, N(R)₂, N(R)C(O)R, N(R)C(O)C(O)R, N(R)C(O)N(R)₂, N(R)C(O)OR, N(R)S(O)R, N(R)SO₂R, N(R)SO₂OR C(O)OR, OC(O)R, C(O)N(R)₂, OC(O)N(R)₂, or an optionally substituted 3-8 membered saturated, partially unsaturated, or aryl monocyclic ring having 0-4 heteroatoms independently selected from nitrogen, oxygen, or sulfur, or an optionally substituted 8-10 membered saturated, partially unsaturated, or aryl bicyclic ring having 0-4 heteroatoms independently selected from nitrogen, oxygen, or sulfur, or:

two R′ are optionally taken together to form an oxo moiety, an oxime, an optionally substituted hydrazone, an optionally substituted imine, or an optionally substituted 3-8 membered saturated or partially unsaturated ring having 0-4 heteroatoms independently selected from nitrogen, oxygen, or sulfur, or:

two R″ are optionally taken together to form an oxo moiety, an oxime, an optionally substituted hydrazone, an optionally substituted imine, or an optionally substituted 3-8 membered saturated or partially unsaturated ring having 0-4 heteroatoms independently selected from nitrogen, oxygen, or sulfur; and

wherein Q is an optionally substituted C₁₋₁₀ alkylene chain wherein one, two, or three methylene units of Q are optionally and independently replaced by —O—, —N(R)—, —S—, —C(O)—, —OC(O)—, —C(O)O—, —OC(O)O—, —S(O)—, or —S(O)₂—, —OSO₂O—, —N(R)C(O)—, —C(O)NR—, —N(R)C(O)O—, —OC(O)NR—, —N(R)C(O)NR—, or —Cy-, wherein:

each —Cy- is independently a bivalent optionally substituted saturated, partially unsaturated, or aromatic monocyclic or bicyclic ring selected from a 6-10 membered arylene, a 5-10 membered heteroarylene having 1-4 heteroatoms independently selected from oxygen, nitrogen, or sulfur, a 3-8 membered carbocyclylene, or a 3-10 membered heterocyclylene having 1-4 heteroatoms independently selected from oxygen, nitrogen, or sulfur, and:

wherein R¹⁰ of the Q-R¹⁰ moiety is selected from the group consisting of hydrogen, halogen, an optionally substituted C₁₋₁₀ aliphatic, a suitably protected hydroxyl group, a suitably protected thiol group, a suitably protected amino group, an optionally substituted 3-8 membered saturated, partially unsaturated, or aryl monocyclic ring having 0-4 heteroatoms independently selected from nitrogen, oxygen, or sulfur, an optionally substituted 8-10 membered saturated, partially unsaturated, or aryl bicyclic ring having 0-4 heteroatoms independently selected from nitrogen, oxygen, or sulfur, a detectable moiety, a polymer residue, a peptide, a sugar-containing or sugar-like moiety, or:

wherein when R¹⁰ is a ring, R¹⁰ is optionally substituted at any substitutable carbon with 1-5 R¹¹ and at any substitutable nitrogen with R¹²;

each R¹¹ is independently halogen, R, OR, SR, N(R)₂, N(R)C(O)R, N(R)C(O)OR, N(R)C(O)N(R)₂, N(R)SO₂R, N(R)SO₂OR, S(O)R, SO₂R, OSO₂R, C(O)R, CO₂R, OCO₂R, C(O)N(R)₂, or OC(O)N(R)₂, or wherein:

two R¹¹ are optionally taken together to form an oxo moiety or an optionally substituted 3-8 membered saturated or partially unsaturated fused or spirofused ring having 0-4 heteroatoms independently selected from nitrogen, oxygen, or sulfur; and

each R¹² is independently R, OR, S(O)R, SO₂R, OSO₂R, C(O)R, CO₂R, OCO₂R, C(O)N(R)₂, or OC(O)N(R)₂, or wherein:

R¹² and R¹¹ are optionally taken together to form an optionally substituted 3-8 membered saturated or partially unsaturated fused ring having 0-4 heteroatoms independently selected from nitrogen, oxygen, or sulfur.

In certain embodiments, the present invention provides a compound wherein each R⁵ of Ring E is independently T-C(R′)₃, T-C(R′)₂C(R″)₃, OR, or a suitably protected hydroxyl group, wherein Q is an optionally substituted C₁₋₁₀ alkylene chain wherein one, two, or three methylene units of Q are optionally and independently replaced by —O—, —N(R)—, —S—, or —Cy-, and wherein R¹⁰ of the Q-R¹⁰ moiety is selected from the group consisting of hydrogen, halogen, a suitably protected hydroxyl group, a suitably protected thiol group, or a suitably protected amino group.

In certain embodiments, the present invention provides a compound wherein each R⁵ of Ring E is independently T-C(R′)₃, T-C(R′)₂C(R″)₃, OR, or a suitably protected hydroxyl group, wherein Q is an optionally substituted C₁₋₁₀ alkylene chain wherein one, two, or three methylene units of Q are optionally and independently replaced by —O—, —N(R)—, —S—, or —Cy-, and wherein R¹⁰ of the Q-R¹⁰ moiety is a ring optionally substituted at any substitutable carbon with 1-5 R¹¹ and at any substitutable nitrogen with R¹², wherein each R¹¹ is independently selected from halogen, R, OR, SR, N(R)₂, N(R)C(O)R, N(R)C(O)OR, N(R)C(O)N(R)₂, N(R)SO₂R, N(R)SO₂OR, S(O)R, SO₂R, OSO₂R, C(O)R, CO₂R, OCO₂R, C(O)N(R)₂, or OC(O)N(R)₂. In certain embodiments, the compound is as described above and R¹⁰ is an optionally substituted heterocycle. In certain embodiments, the compound is as described above and R¹⁰ is an optionally substituted 5-6 membered heterocycle with 1-3 heteroatoms. In certain embodiments, the compound is as described above and R¹⁰ is an optionally substituted 6 membered heterocycle with 2 heteroatoms. In certain embodiments, the compound is as described above and R¹⁰ is optionally substituted morpholine.

In certain embodiments, the present invention provides a compound wherein each R⁵ of Ring E is independently T-C(R′)₃, T-C(R′)₂C(R″)₃, OR, or a suitably protected hydroxyl group, wherein Q is an optionally substituted C₁₋₁₀ alkylene chain wherein one, two, or three methylene units of Q are optionally and independently replaced by —O—, —N(R)—, —S—, or —Cy-, and wherein R¹⁰ of the Q-R¹⁰ moiety is a sugar-containing or sugar-like moiety.

In certain embodiments, the present invention provides a compound wherein each of Q-R¹⁰ and R⁵ are as described in any one of the above embodiments and the compound is of the general formula:

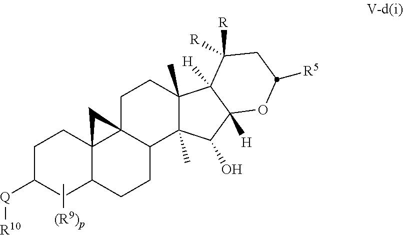

wherein each variable is defined above and in classes and subclasses herein.

In certain embodiments, the present invention provides a compound wherein each of Q-R¹⁰ and R⁵ are as described in any one of the above embodiments and the compound is of the general formula:

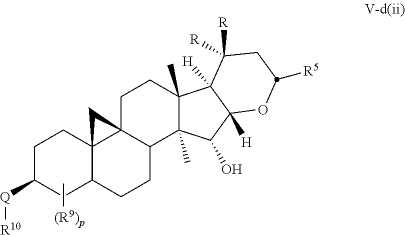

wherein each variable is defined above and in classes and subclasses herein.

In certain embodiments, the present invention provides a compound of the general formula:

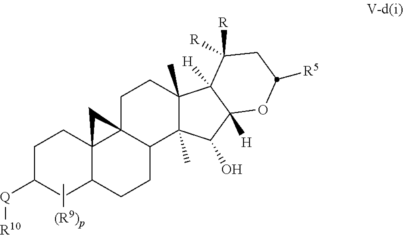

wherein R, R⁹, and p are as defined above and in classes and subclasses herein, and wherein:

R¹⁰ is hydrogen and Q is an optionally substituted C₂₋₁₀ alkylene chain wherein two or three methylene units are independently replaced by —OC(O)NR— and —Cy-; or

R¹⁰ is hydrogen and Q is an optionally substituted C₂₋₁₀ alkylene chain wherein two or three methylene units are independently replaced by —OC(O)— and —Cy-; or

R¹⁰ is selected from the group consisting of tetrahydropyranyl, tetrahydrofuranyl, morpholinyl, thiomorpholinyl, piperidinyl, piperazinyl, pyrrolidinyl, tetrahydrothiophenyl, and tetrahydrothiopyranyl, wherein each ring is optionally substituted at any substitutable carbon with 1-5 R¹¹ and at any substitutable nitrogen with R¹²; and wherein:

R⁵ of Ring E is independently T-C(R′)₃, T-C(R′)₂C(R″)₃, OR, a suitably protected hydroxyl group, SR, a suitably protected thiol group, S(O)R, SO₂R, OSO₂R, N(R)₂, a suitably protected amino group, N(R)C(O)R, N(R)C(O)C(O)R, N(R)C(O)N(R)₂, N(R)C(O)OR, C(O)OR, OC(O)R, C(O)N(R)₂, or OC(O)N(R)₂, an optionally substituted 3-8 membered saturated, partially unsaturated, or aryl monocyclic ring having 0-4 heteroatoms independently selected from nitrogen, oxygen, or sulfur, an optionally substituted 8-10 membered saturated, partially unsaturated, or aryl bicyclic ring having 0-4 heteroatoms independently selected from nitrogen, oxygen, or sulfur;

each T is independently a valence bond or an optionally substituted straight or branched, saturated or unsaturated, C₁₋₆ alkylene chain wherein up to two methylene units of T are optionally and independently replaced by —O—, —N(R)—, —S—, —C(O)—, —S(O)—, or —S(O)₂—;

each R′ and R″ is independently selected from halogen, R, OR, SR, S(O)R, SO₂R, OSO₂R, N(R)₂, N(R)C(O)R, N(R)C(O)C(O)R, N(R)C(O)N(R)₂, N(R)C(O)OR, N(R)S(O)R, N(R)SO₂R, N(R)SO₂OR C(O)OR, OC(O)R, C(O)N(R)₂, OC(O)N(R)₂, or an optionally substituted 3-8 membered saturated, partially unsaturated, or aryl monocyclic ring having 0-4 heteroatoms independently selected from nitrogen, oxygen, or sulfur, or an optionally substituted 8-10 membered saturated, partially unsaturated, or aryl bicyclic ring having 0-4 heteroatoms independently selected from nitrogen, oxygen, or sulfur, or:

two R′ are optionally taken together to form an oxo moiety, an oxime, an optionally substituted hydrazone, an optionally substituted imine, or an optionally substituted 3-8 membered saturated or partially unsaturated ring having 0-4 heteroatoms independently selected from nitrogen, oxygen, or sulfur, or:

two R″ are optionally taken together to form an oxo moiety, an oxime, an optionally substituted hydrazone, an optionally substituted imine, or an optionally substituted 3-8 membered saturated or partially unsaturated ring having 0-4 heteroatoms independently selected from nitrogen, oxygen, or sulfur.

In certain embodiments, the present invention provides a compound of the general formula:

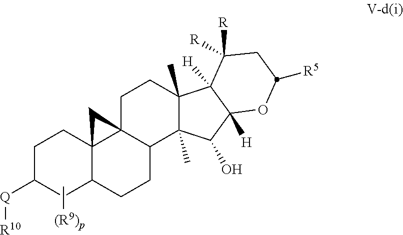

wherein R, R⁹, and p are as defined above and in classes and subclasses herein, and wherein:

R¹⁰ is hydrogen and Q is an optionally substituted C₂₋₁₀ alkylene chain wherein two or three methylene units are independently replaced by —OC(O)NR— and —Cy-; or

R¹⁰ is hydrogen and Q is an optionally substituted C₂₋₁₀ alkylene chain wherein two or three methylene units are independently replaced by —OC(O)— and —Cy-; or

R¹⁰ is selected from the group consisting of tetrahydropyranyl, tetrahydrofuranyl, morpholinyl, thiomorpholinyl, piperidinyl, piperazinyl, pyrrolidinyl, tetrahydrothiophenyl, and tetrahydrothiopyranyl, wherein each ring is optionally substituted at any substitutable carbon with 1-5 R¹¹ and at any substitutable nitrogen with R¹²; and wherein:

R⁵ is of any of the following formulae:

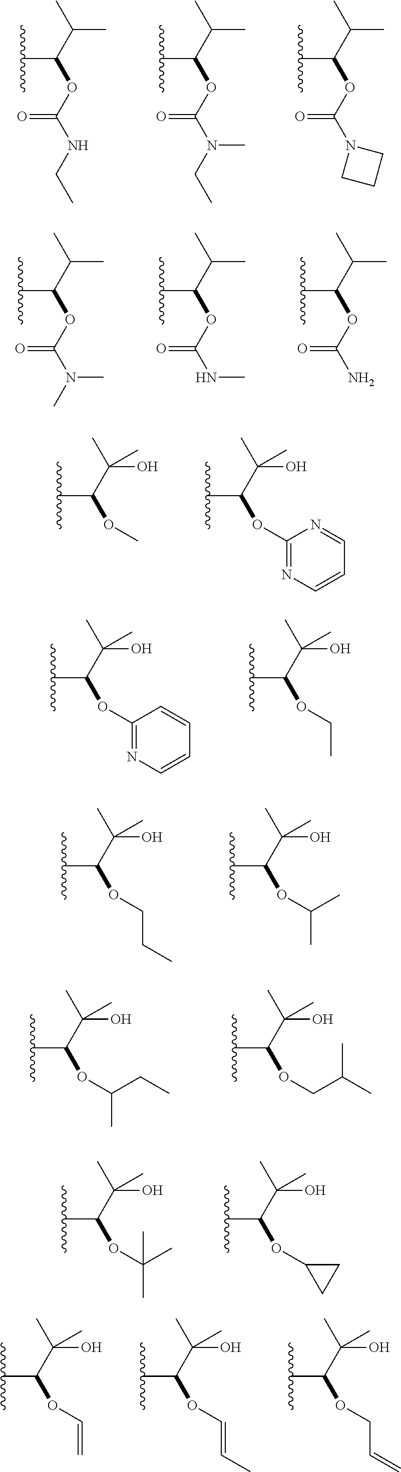

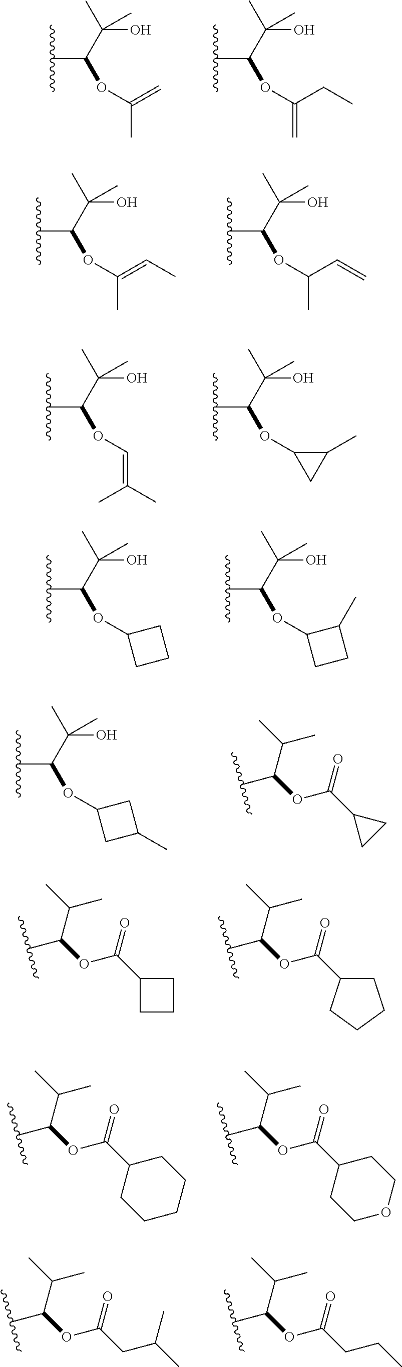

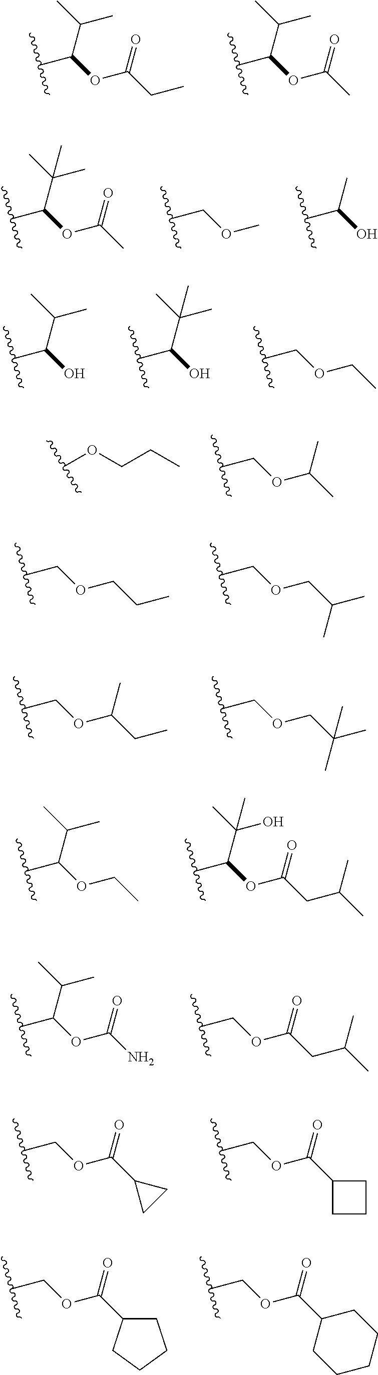

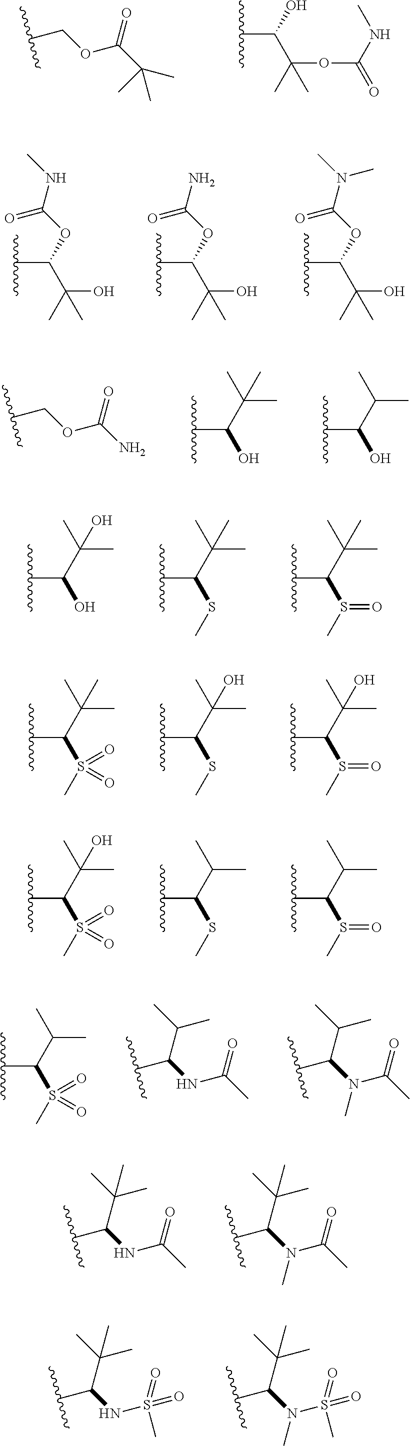

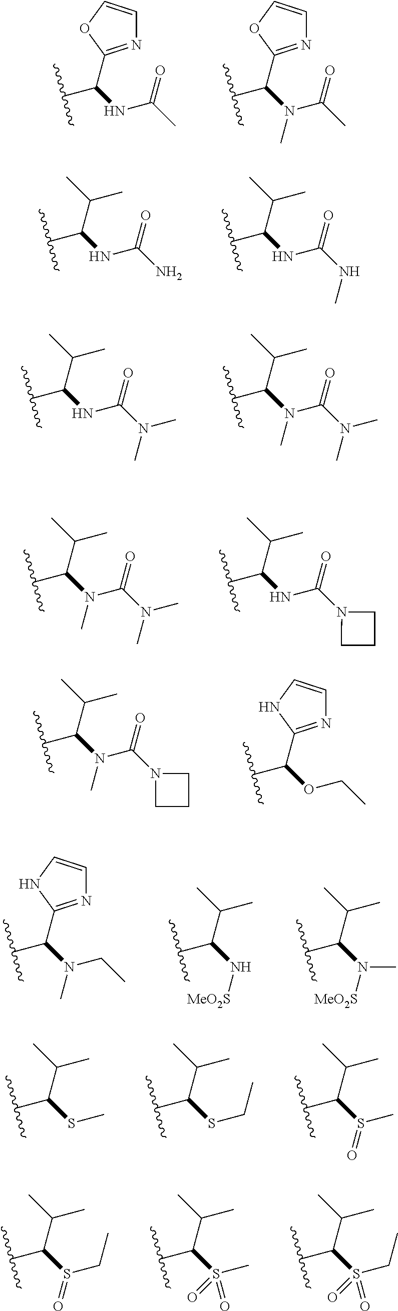

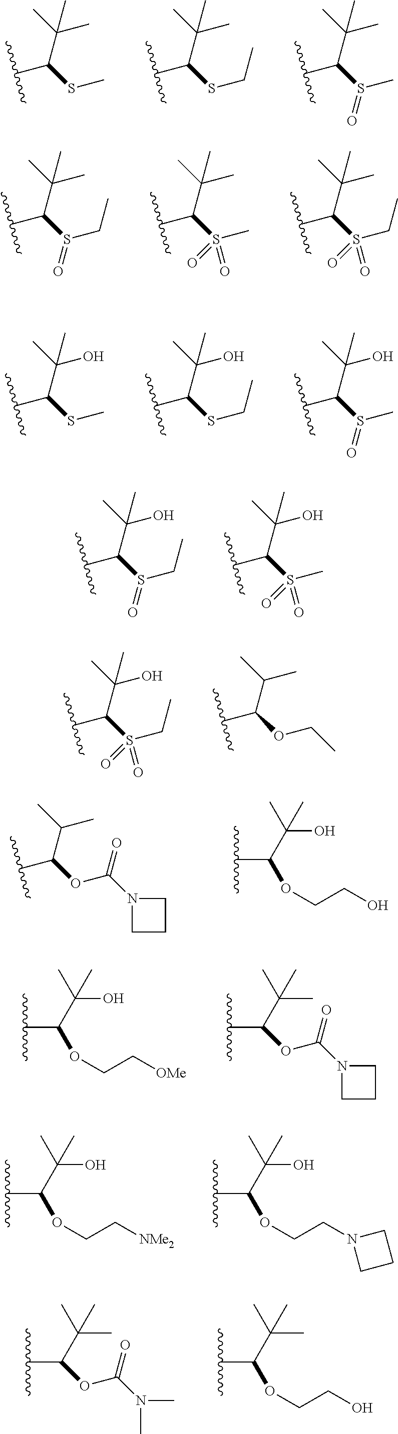

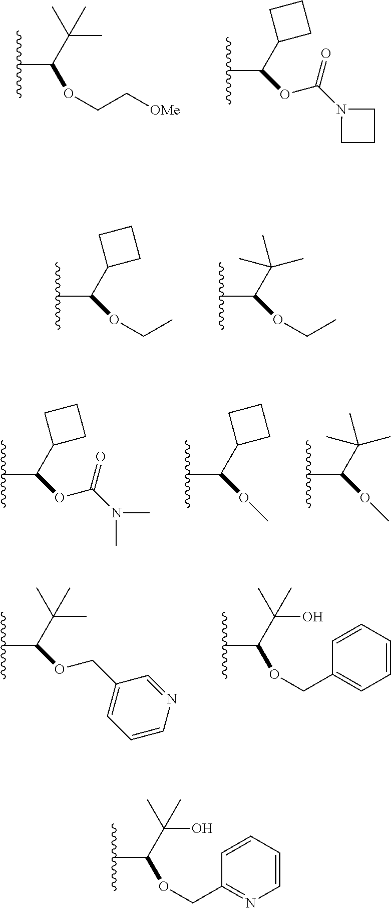

In certain embodiments, the present invention provides a compound of the general formula:

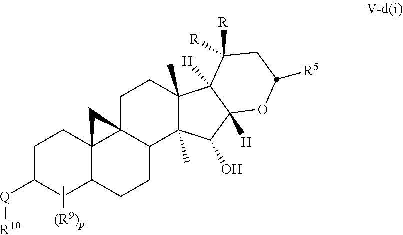

wherein R, R⁹, and p are as defined above and in classes and subclasses herein, and wherein: R¹⁰ is as depicted below:

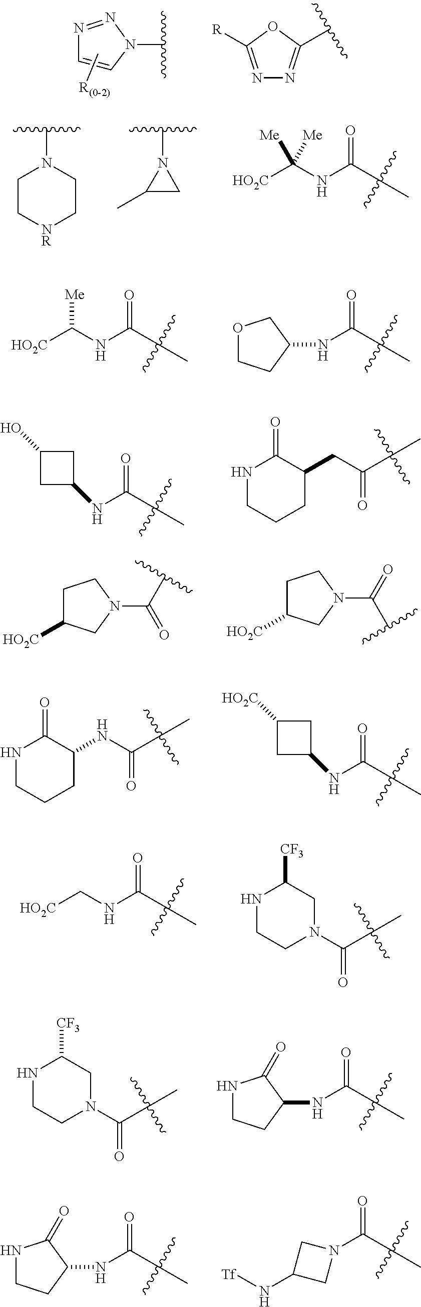

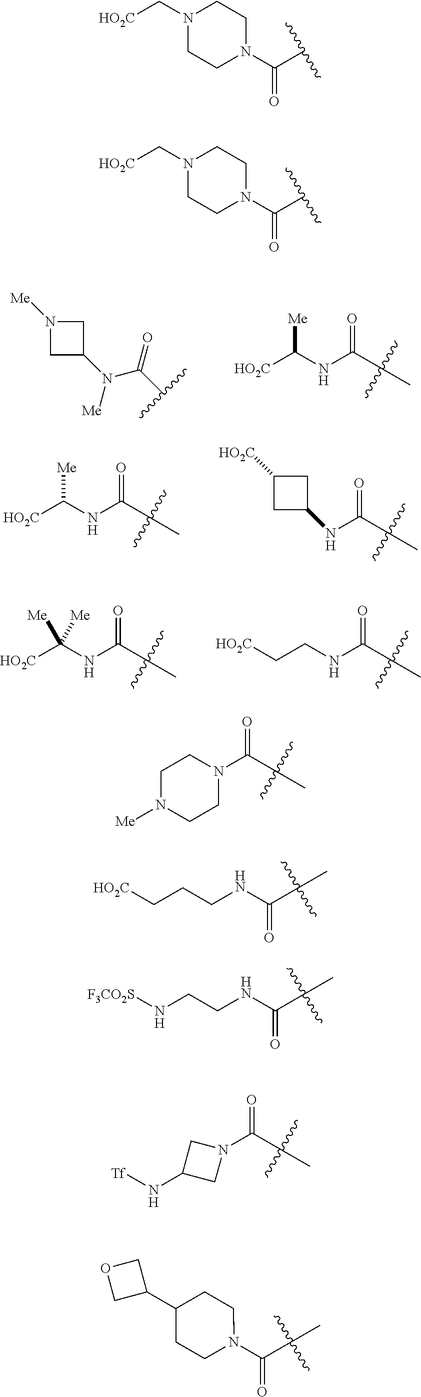

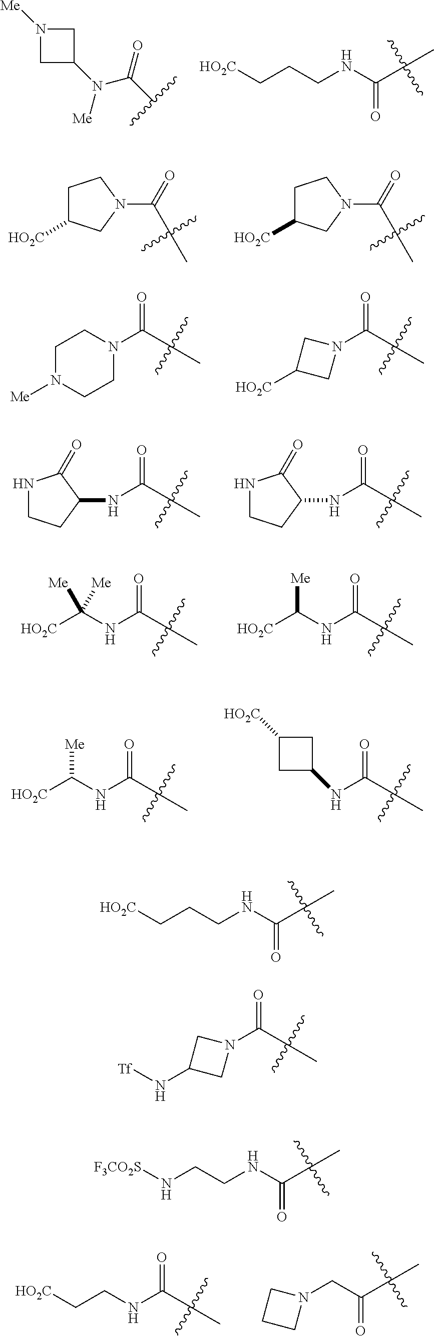

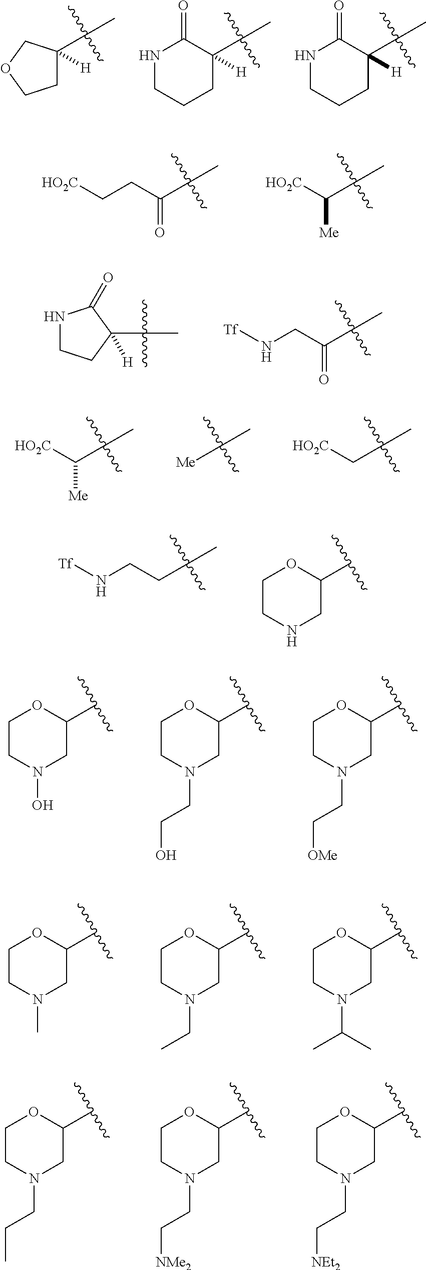

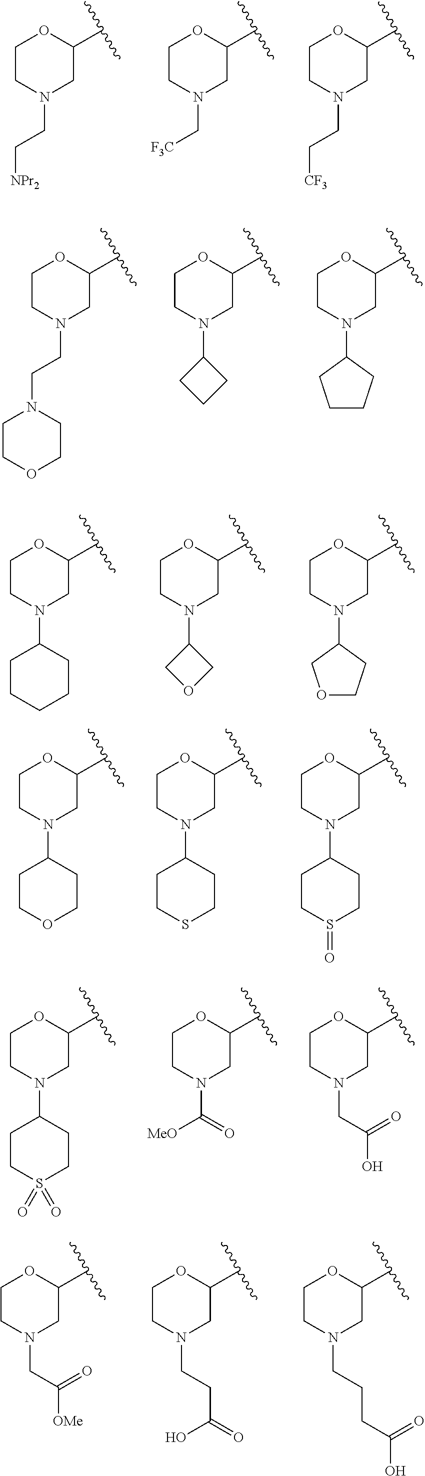

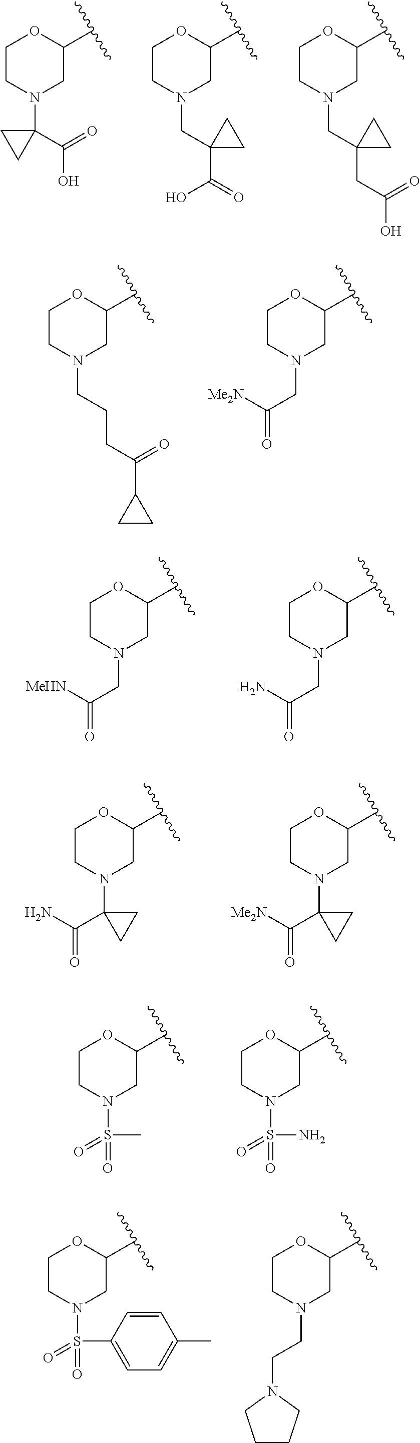

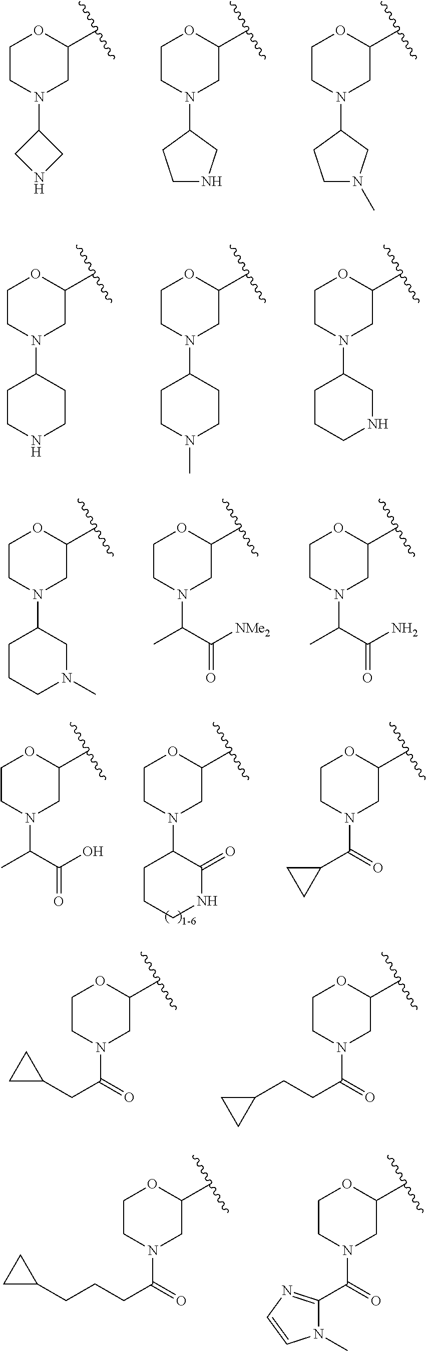

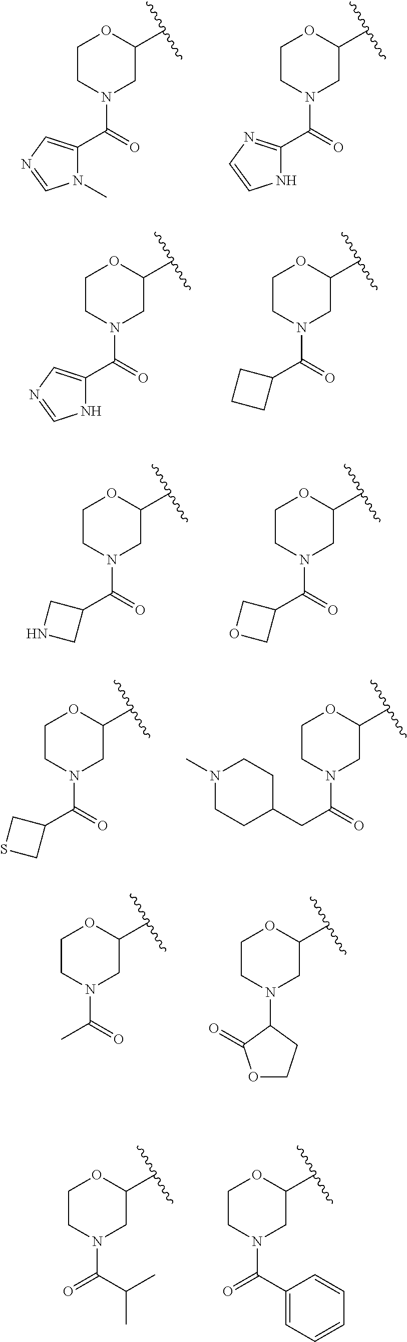

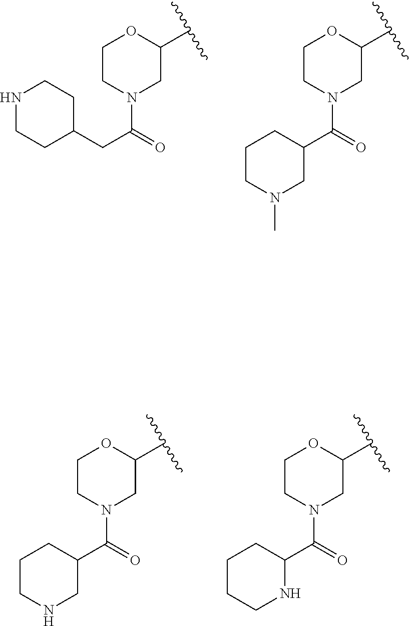 [SMILES: *C1CN(C(=O)CC2CCNCC2)CCO1 ; *C1CN(C(=O)C2CCCN(C)C2)CCO1 ; *C1CN(C(=O)C2CCCNC2)CCO1 ; *C1CN(C(=O)C2CCCCN2)CCO1]

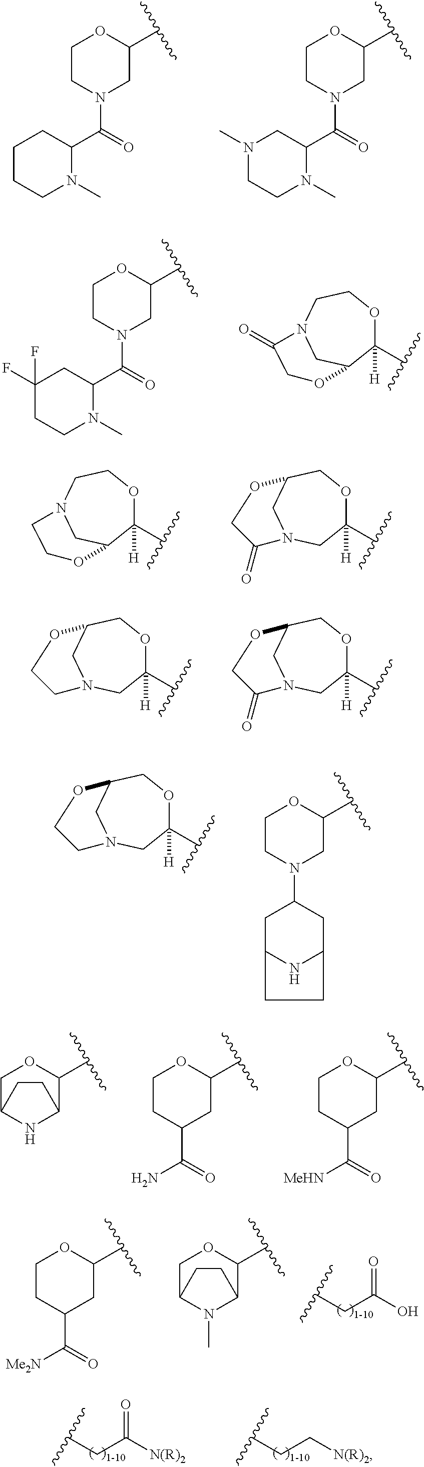

or wherein Q-R¹⁰ is as depicted below:

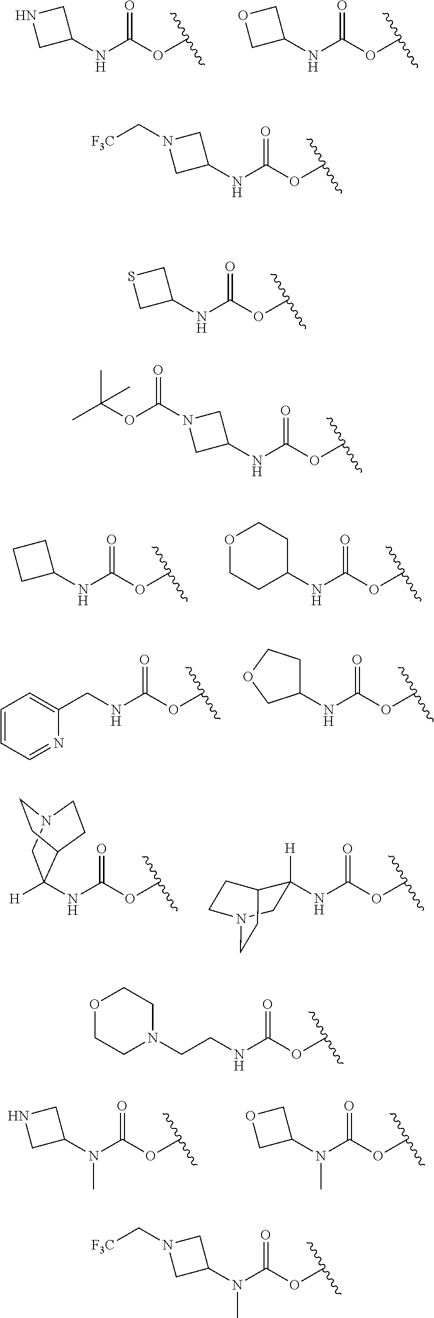

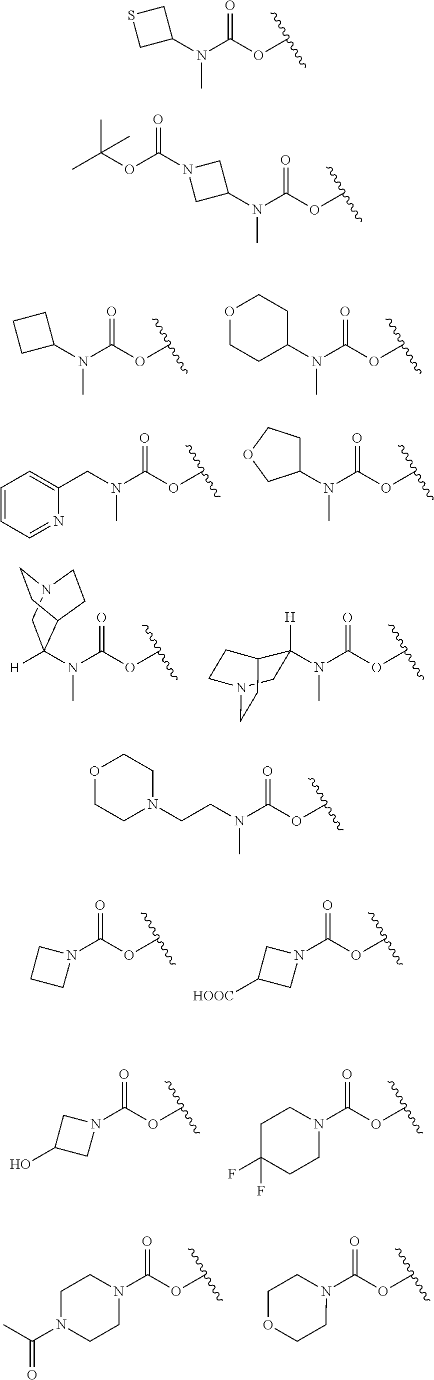

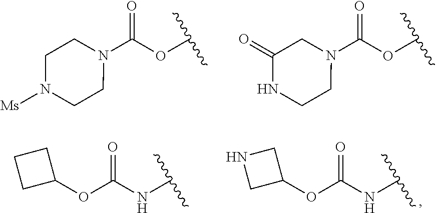 [SMILES: *OC(=O)N1CCN(S(C)(=O)=O)CC1 ; *OC(=O)N1CCNC(=O)C1 ; *NC(=O)OC1CCC1 ; *NC(=O)OC1CNC1]

and wherein:

R⁵ of Ring E is independently T-C(R′)₃, T-C(R′)₂C(R″)₃, OR, a suitably protected hydroxyl group, SR, a suitably protected thiol group, S(O)R, SO₂R, OSO₂R, N(R)₂, a suitably protected amino group, N(R)C(O)R, N(R)C(O)C(O)R, N(R)C(O)N(R)₂, N(R)C(O)OR, C(O)OR, OC(O)R, C(O)N(R)₂, or OC(O)N(R)₂, an optionally substituted 3-8 membered saturated, partially unsaturated, or aryl monocyclic ring having 0-4 heteroatoms independently selected from nitrogen, oxygen, or sulfur, an optionally substituted 8-10 membered saturated, partially unsaturated, or aryl bicyclic ring having 0-4 heteroatoms independently selected from nitrogen, oxygen, or sulfur;

each T is independently a valence bond or an optionally substituted straight or branched, saturated or unsaturated, C₁₋₆ alkylene chain wherein up to two methylene units of T are optionally and independently replaced by —O—, —N(R)—, —S—, —C(O)—, —S(O)—, or —S(O)₂—;

each R′ and R″ is independently selected from halogen, R, OR, SR, S(O)R, SO₂R, OSO₂R, N(R)₂, N(R)C(O)R, N(R)C(O)C(O)R, N(R)C(O)N(R)₂, N(R)C(O)OR, N(R)S(O)R, N(R)SO₂R, N(R)SO₂OR C(O)OR, OC(O)R, C(O)N(R)₂, OC(O)N(R)₂, or an optionally substituted 3-8 membered saturated, partially unsaturated, or aryl monocyclic ring having 0-4 heteroatoms independently selected from nitrogen, oxygen, or sulfur, or an optionally substituted 8-10 membered saturated, partially unsaturated, or aryl bicyclic ring having 0-4 heteroatoms independently selected from nitrogen, oxygen, or sulfur, or:

two R′ are optionally taken together to form an oxo moiety, an oxime, an optionally substituted hydrazone, an optionally substituted imine, or an optionally substituted 3-8 membered saturated or partially unsaturated ring having 0-4 heteroatoms independently selected from nitrogen, oxygen, or sulfur, or:

two R″ are optionally taken together to form an oxo moiety, an oxime, an optionally substituted hydrazone, an optionally substituted imine, or an optionally substituted 3-8 membered saturated or partially unsaturated ring having 0-4 heteroatoms independently selected from nitrogen, oxygen, or sulfur.

In certain embodiments, the present invention provides a compound of the general formula:

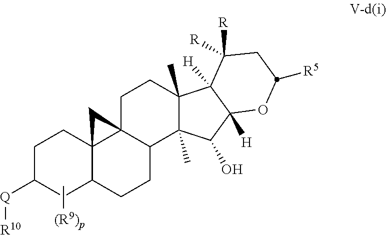

wherein R, R⁹, and p are as defined above and in classes and subclasses herein, and wherein: R¹⁰ is as depicted below:

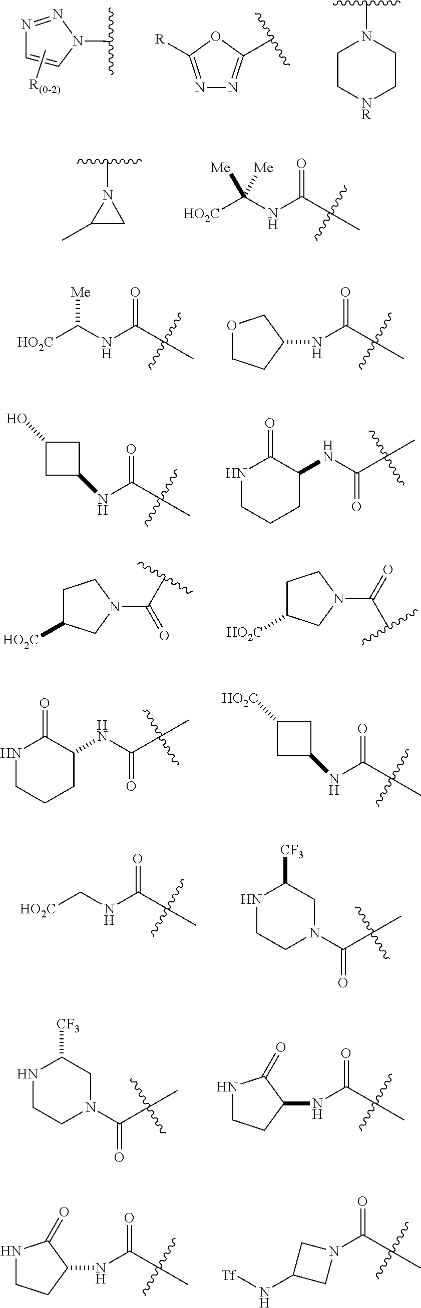

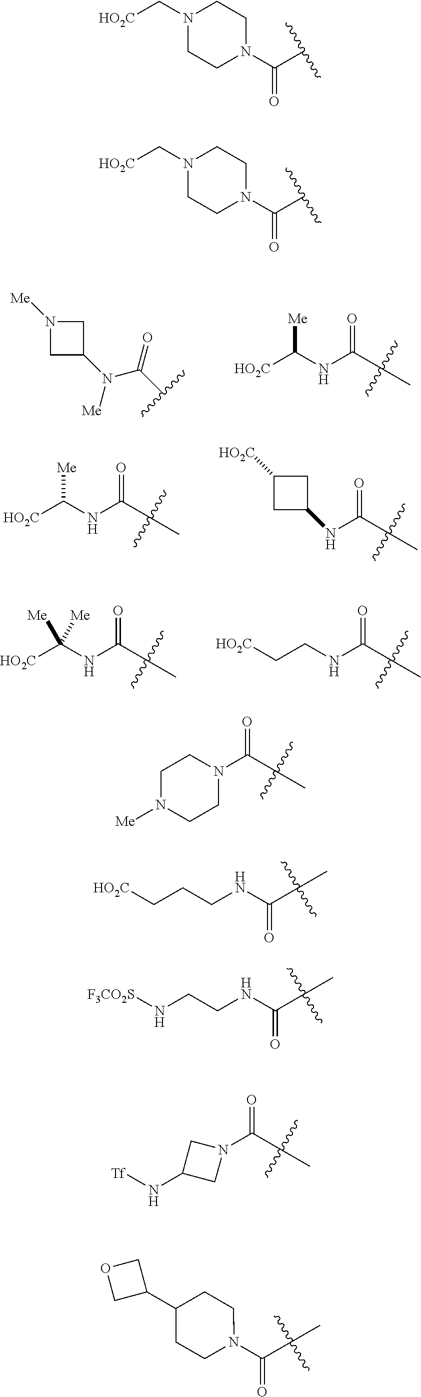

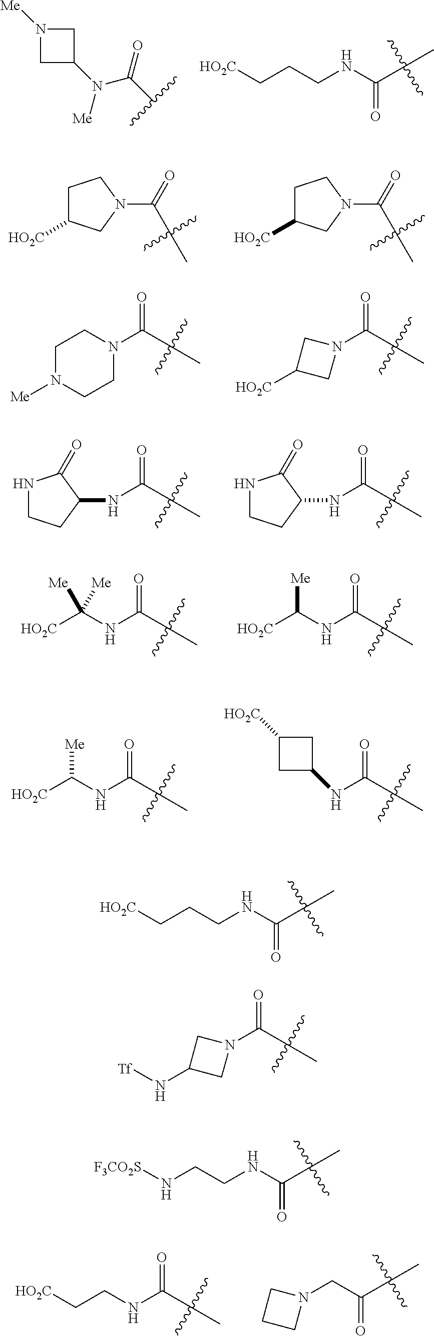

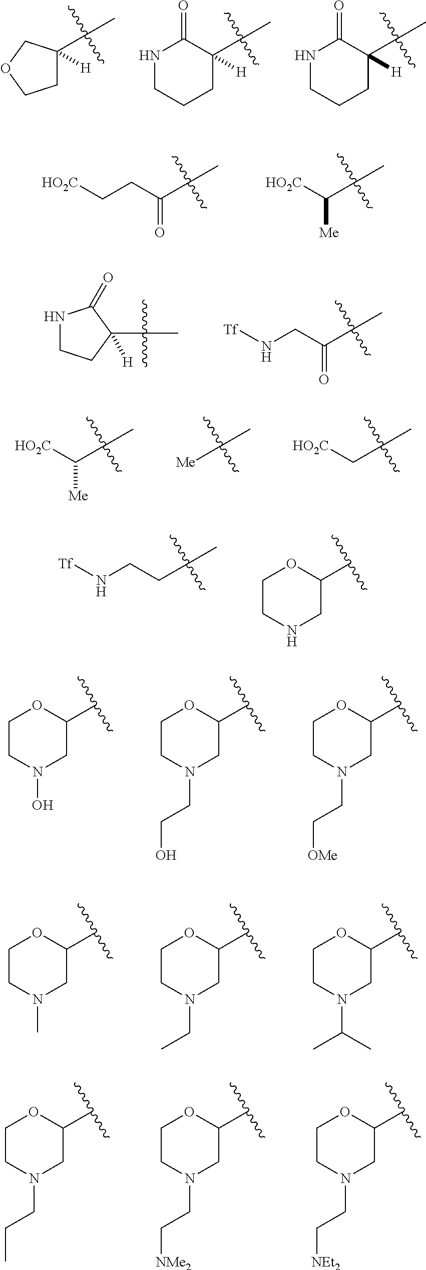

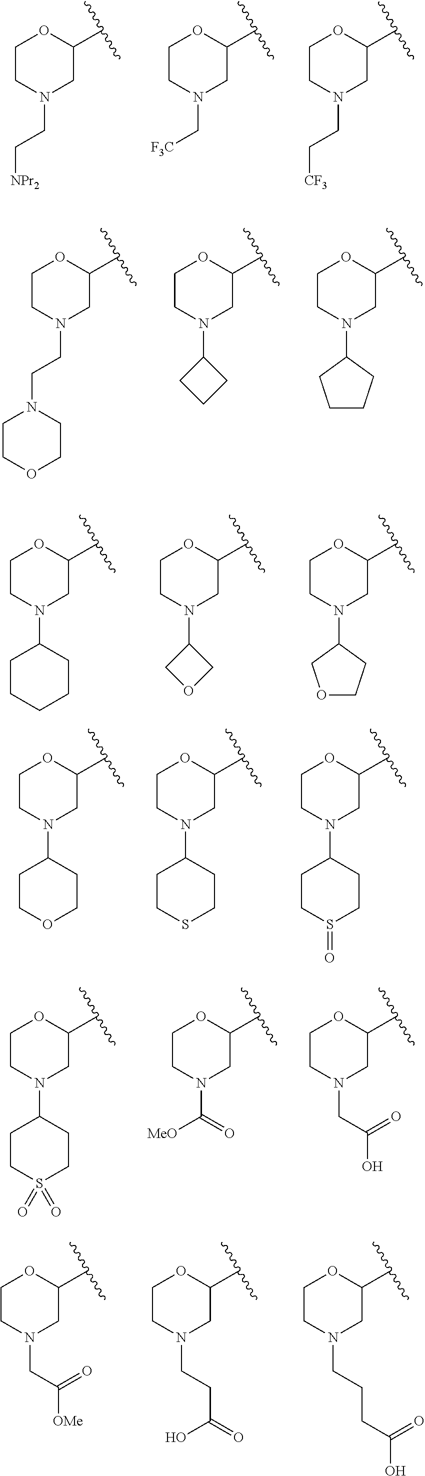

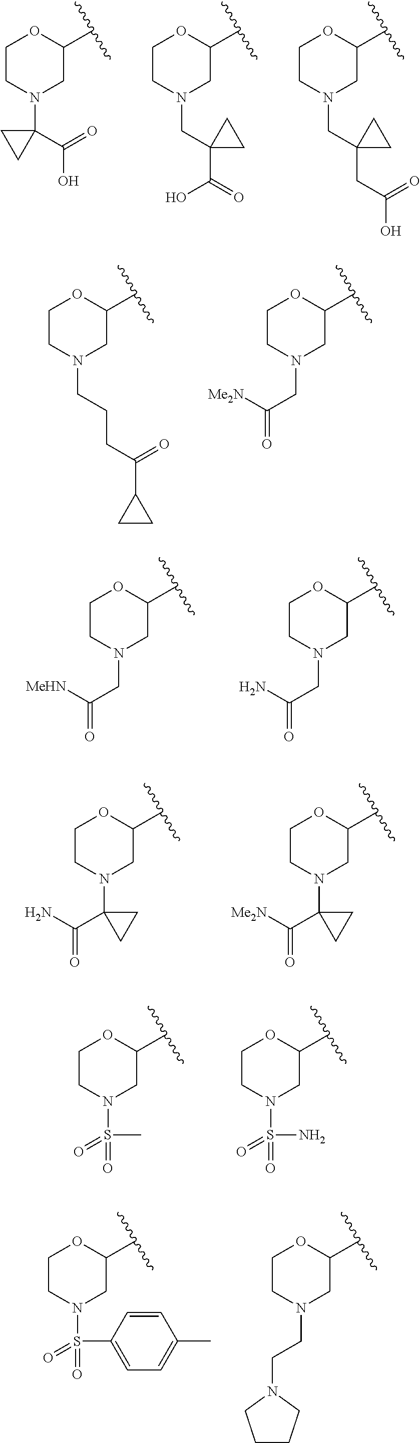

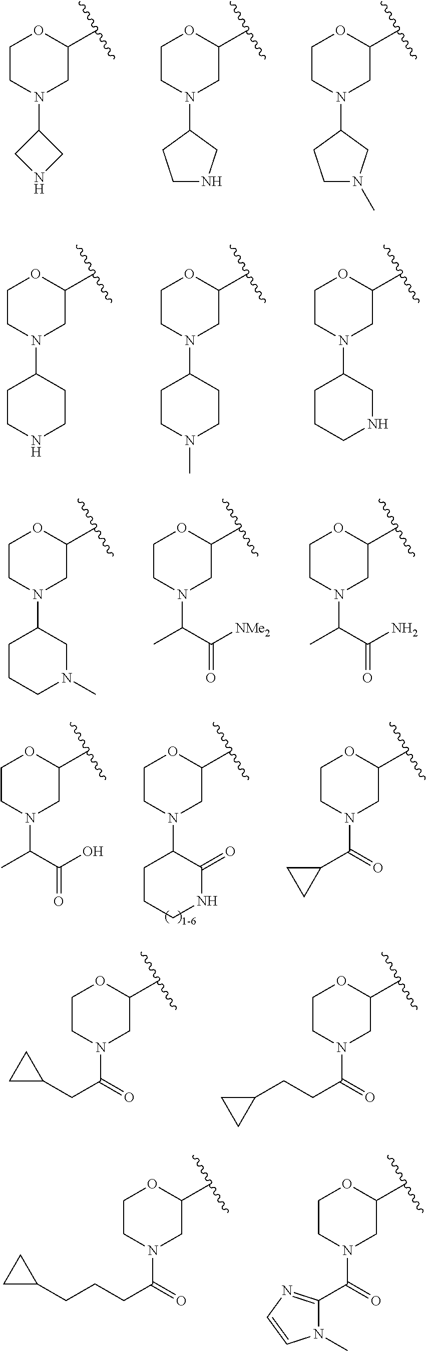

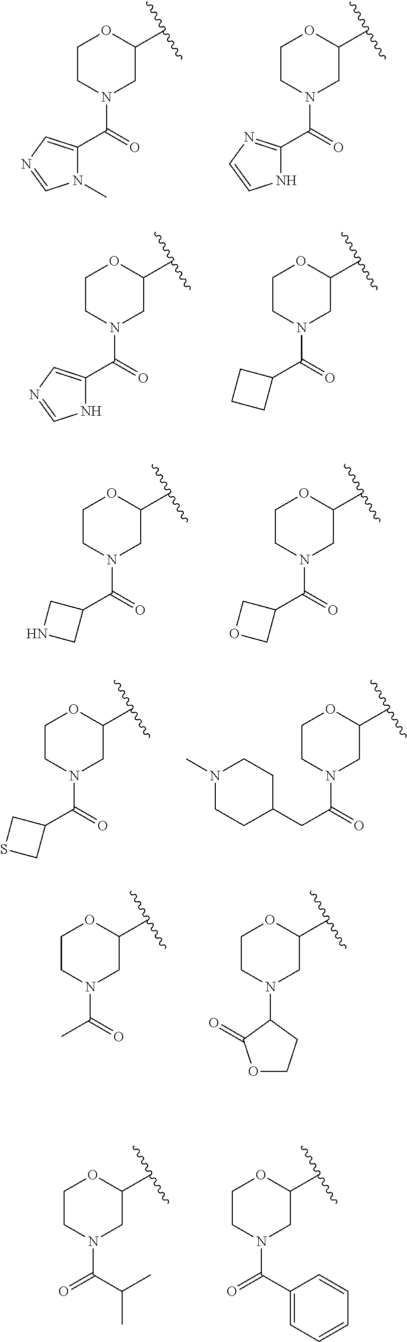

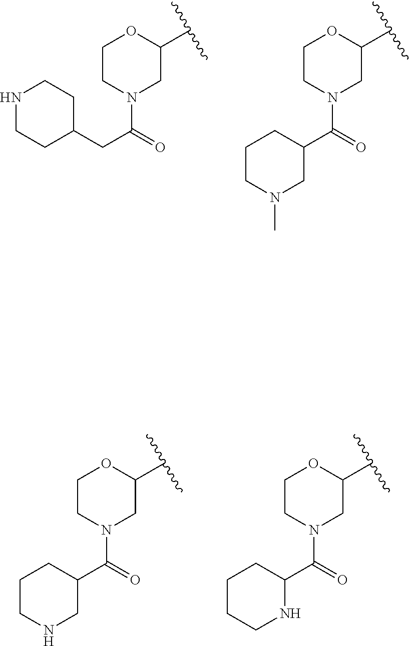 [SMILES: *C1CN(C(=O)CC2CCNCC2)CCO1 ; *C1CN(C(=O)C2CCCN(C)C2)CCO1 ; *C1CN(C(=O)C2CCCNC2)CCO1 ; *C1CN(C(=O)C2CCCCN2)CCO1]

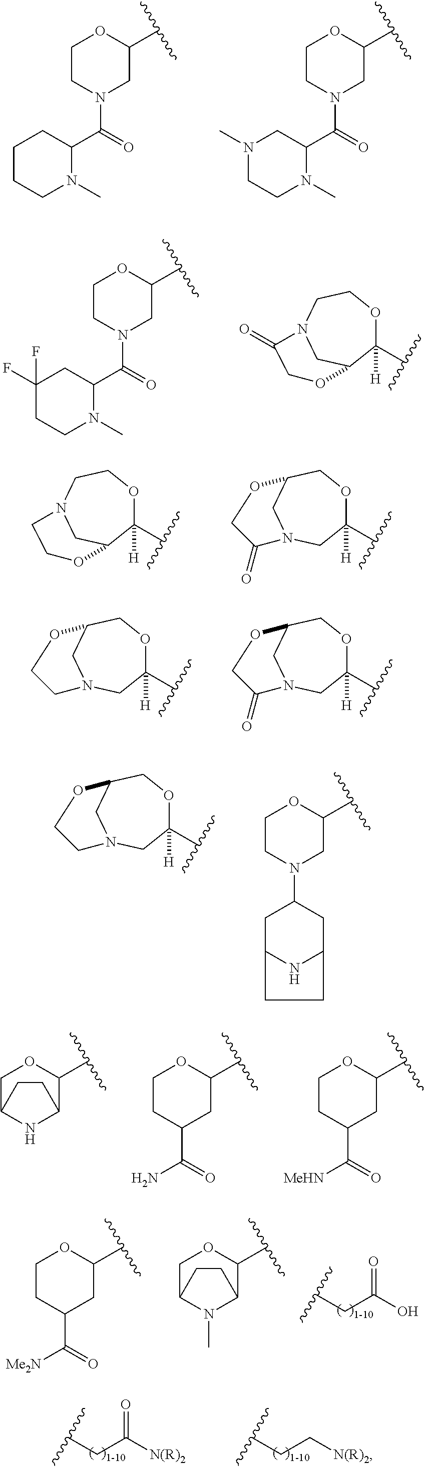

or wherein Q-R¹⁰ is as depicted below:

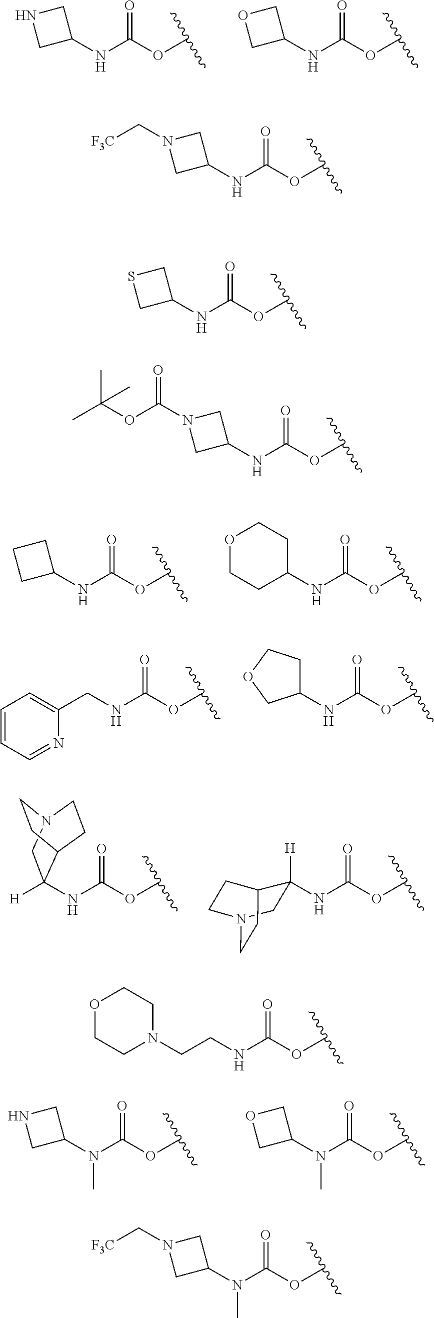

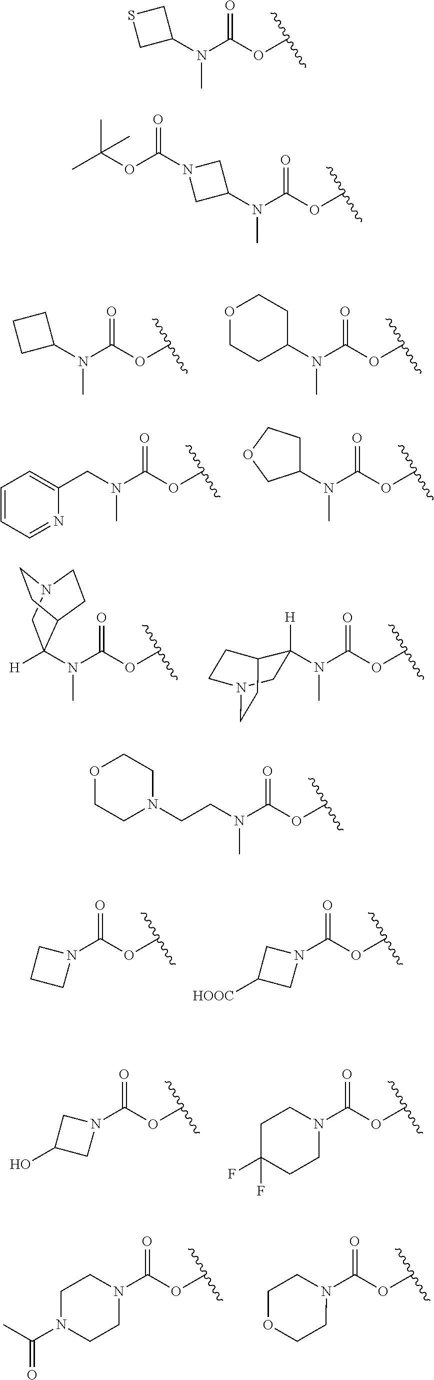

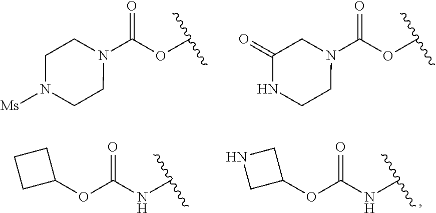 [SMILES: *OC(=O)N1CCN(S(C)(=O)=O)CC1 ; *OC(=O)N1CCNC(=O)C1 ; *NC(=O)OC1CCC1 ; *NC(=O)OC1CNC1]

and wherein R⁵ is as depicted below:

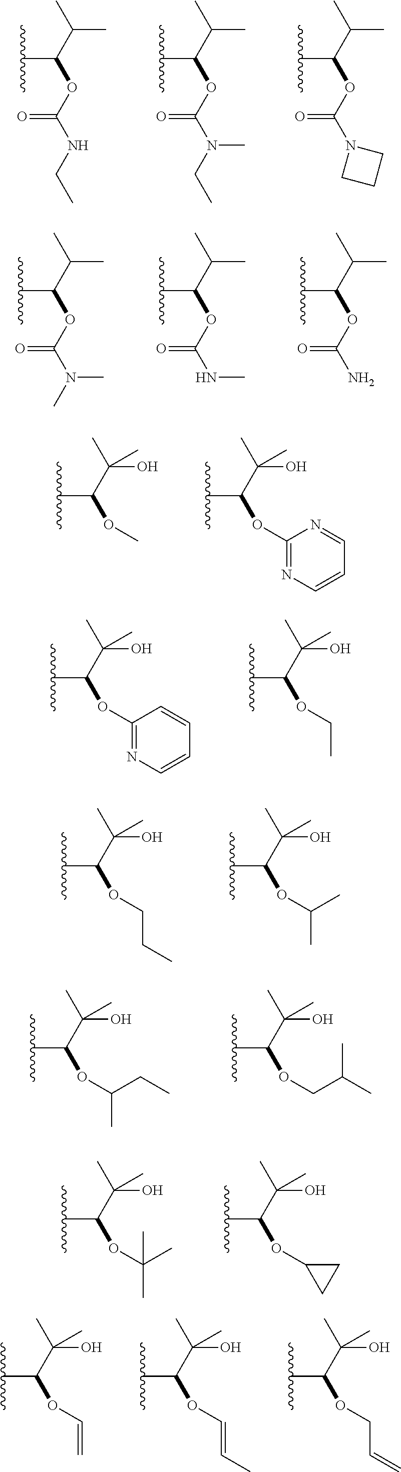

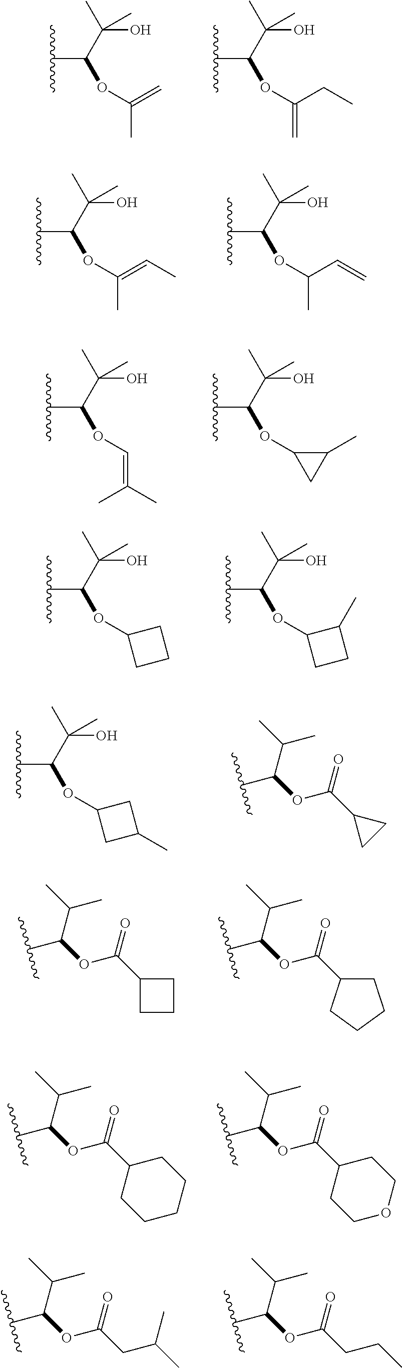

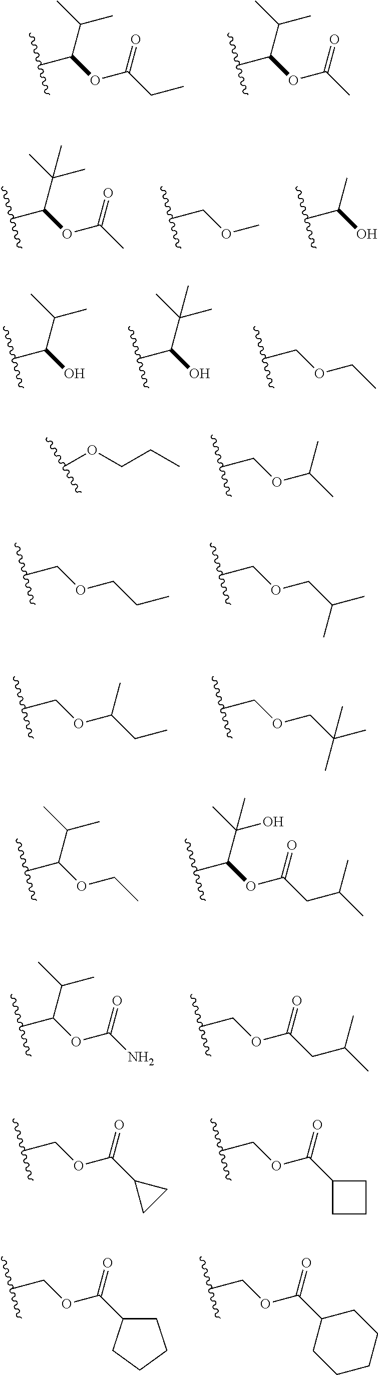

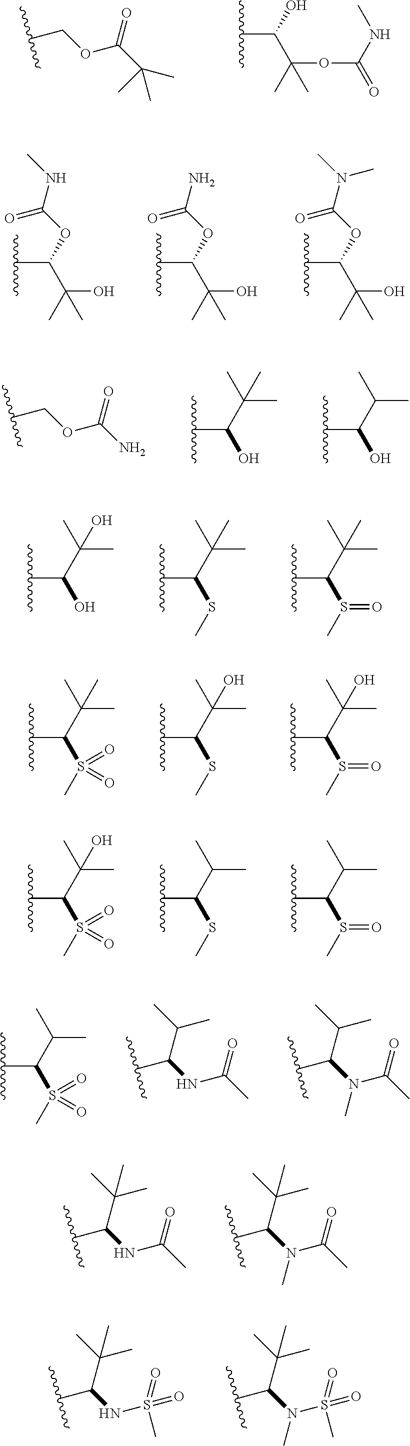

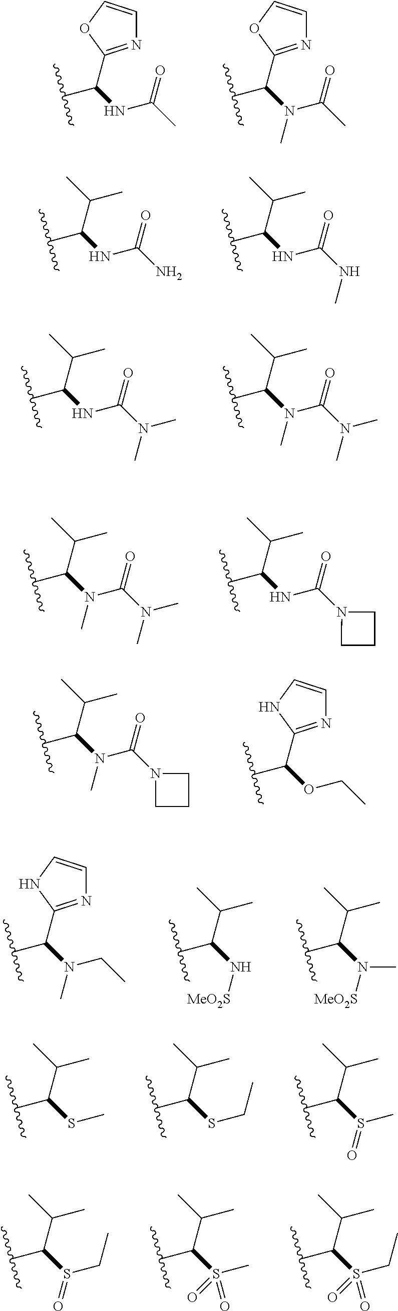

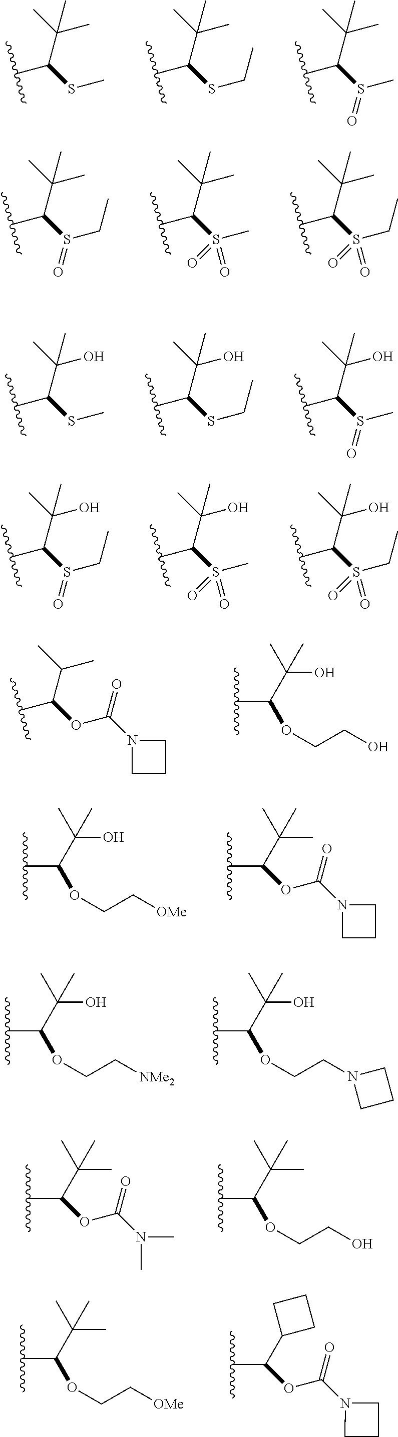

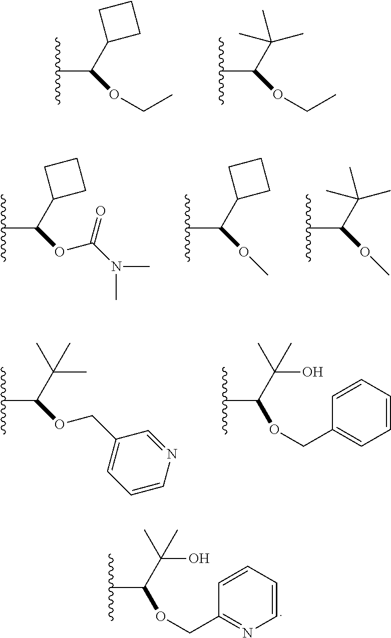

One of skill in the art would recognize that compounds containing Q-R¹⁰ and R⁵ moieties can be synthesized via certain common synthetic intermediates described above and herein and that the scope of combinations of Q-R¹⁰ and R⁵, and thus the scope of compounds contemplated and described herein, is extensive.

### 13. General Methods of Providing the Present Compounds

The compounds of this invention may be prepared or isolated in general by synthetic and/or semi-synthetic methods known to those skilled in the art for analogous compounds and by methods described in detail in the Examples, below.

Provided compounds are prepared by methods known to one of ordinary skill in the art and including methods illustrated in Schemes 1-6, below. Unless otherwise noted, all variables are as defined above and in classes and subclasses herein.

In the Schemes below, where a particular protecting group, leaving group, or transformation condition is depicted, one of ordinary skill in the art will appreciate that other protecting groups, leaving groups, and transformation conditions are also suitable and are contemplated. Such groups and transformations are described in detail in March's Advanced Organic Chemistry Reactions, Mechanisms, and Structure, M. B. Smith and J. March, 5^{th} Edition, John Wiley & Sons, 2001, Comprehensive Organic Transformations, R. C. Larock, 2^{nd} Edition, John Wiley & Sons, 1999, and Protecting Groups in Organic Synthesis, T. W. Greene and P. G. M. Wuts, 3^{rd} edition, John Wiley & Sons, 1999, the entirety of each of which is hereby incorporated herein by reference.

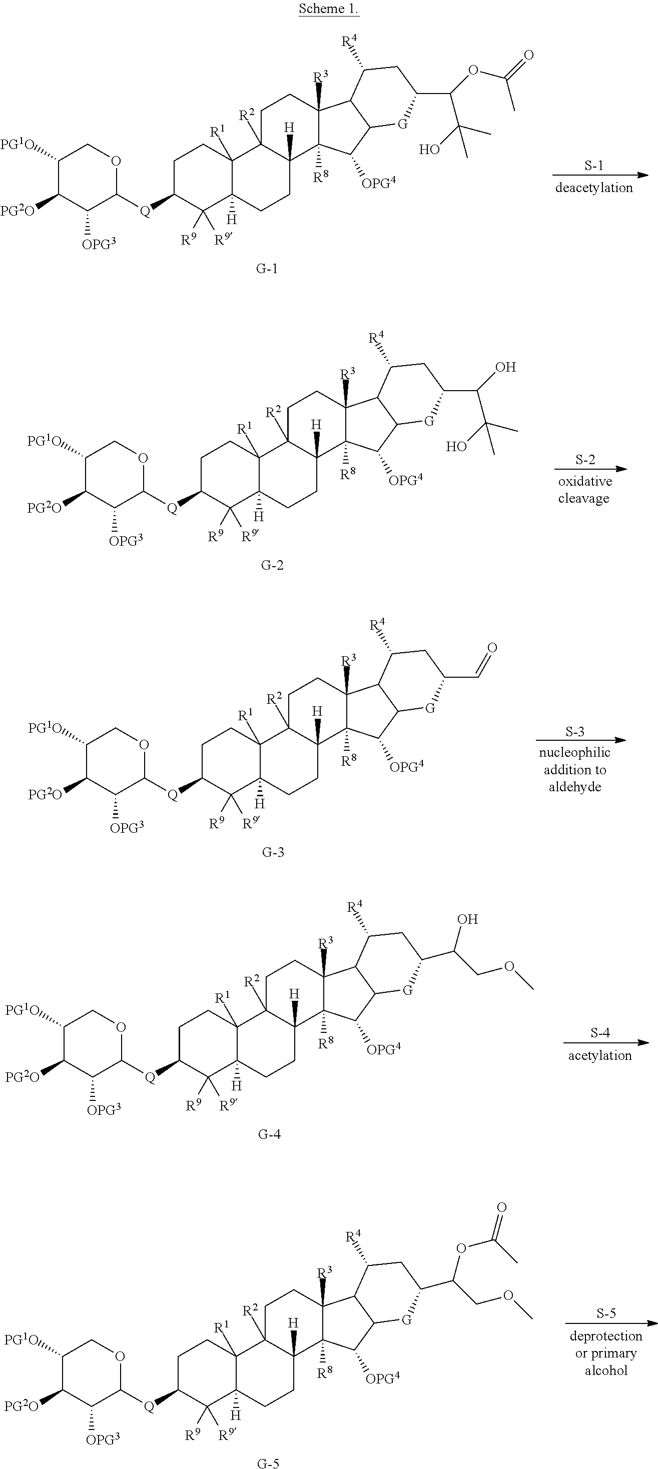

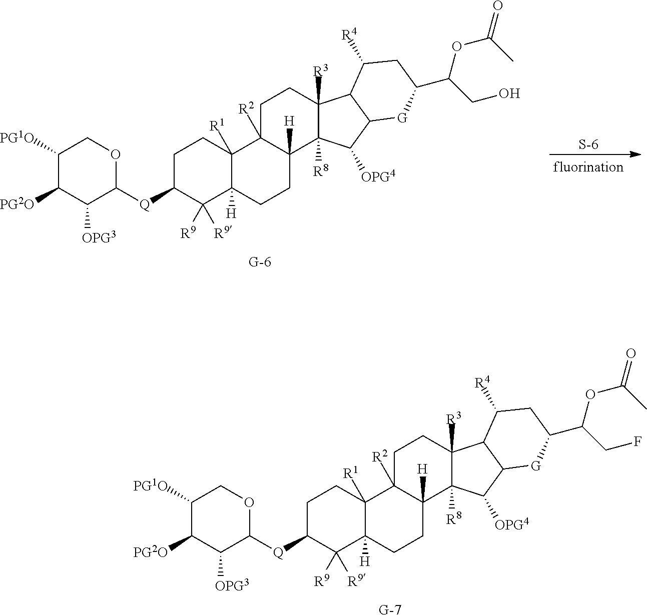

In some embodiments, compounds are synthesized as depicted in Scheme 1 above, wherein PG¹, PG², PG³, and PG⁴ are each independently hydroxy protecting groups. In some embodiments, G-7 is synthesized from G-1. S-1 illustrates the deacetylation of polyol G-1 to afford the corresponding free alcohol G-2. In some embodiments, G-1 is deacetylated under basic conditions in a protic solvent. In certain embodiments, the base is a carbonate base such as, for instance, potassium carbonate, and the protic solvent is an alcoholic solvent such as methanol. One of ordinary skill in the art would recognize that alternative carbonate bases (e.g., sodium, cesium) and alternative alcoholic solvents (ethanol, isopropanol) are also contemplated herein. Work-up and purification of the reaction affords des-acetate G-2.

In step S-2 above, oxidative cleavage of the diol moiety of G-2 using an appropriate oxidant furnishes aldehyde G-3. In some embodiments, the oxidant is a hypervalent iodide and oxidation takes place in protic media. In certain embodiments, exposure of G-2 to sodium periodate in water provides aldehyde G-3.

As shown in step S-3 above, aldehyde G-3 undergoes nucleophilic addition to install the ether-containing side chain and afford the corresponding alcohol G-4. In some embodiments, the nucleophile is a stannane premixed in an ethereal solvent (e.g., tetrahydrofuran (THF)) with an organolithium reagent (e.g., n-butyllithium) to form the active nucleophile. In certain embodiments, the stannane contains a desired transferable group such as, for instance methoxy methyl. Dropwise addition of the preformed nucleophile (e.g., lithiomethoxymethane) to aldehyde G-3 furnishes the corresponding alcohol G-4.

As shown in step S-4 above, alcohol G-4 is then acetylated to produce acetate G-5. In some embodiments, acetylation occurs in a polar aprotic solvent. In certain embodiments, the solvent is a halogenated solvent such as dichloromethane. Exposure of alcohol G-4 to an acetylating reagent affords acetate G-5. In certain embodiments, the acetylating reagent is acetic anhydride and an additional amine catalyst (e.g., dimethylaminopyridine (DMAP)) is used to facilitate the transformation. In other embodiments, an alternative acetylating reagent may be used with or without an additional catalyst. Exemplary such other reagents include, for example, acetyl halides such as acetyl chloride.

As shown in step S-5 above, selective cleavage of the newly installed pendant ether-containing side chain of G-5 reveals primary alcohol intermediate G-6. In some embodiments, cleavage of the G-5 ether occurs upon exposure to acid at room temperature. In certain embodiments, alcohol G-6 is generated using a Bronsted acid (e.g., hydrochloric acid (HCl)).

As shown in step S-6 above, fluorination of G-6 via displacement of the primary alcohol affords G-7. In some embodiments, displacement of the primary alcohol occurs upon exposure of G-6 to a nucleophilic fluorinating agent (e.g., CsF, KF, tetraalkylammonium fluorides, HF-amine complexes, fluoroborates and analogs thereof) in an aprotic solvent (e.g., n-methylpyrrolidine (NMP), dimethylformamide (DMF), dimethylacetamide (DMA), sulpholane, glyme, acetonitrile, or dichloromethane). In certain embodiments, fluorination occurs in the presence of a suitable crown ether and/or is preceeded by first transforming the alcohol into a more reactive leaving group. In other embodiments, fluorination occurs using sulfur tetrafluoride/HF, or an equivalent thereof (e.g., diethylaminosulfur trifluoride (DAST) or bis(2-methoxyethyl)aminosulfur trifluoride (BAST)). In certain embodiments, the fluorinated intermediate is subsequently subjected to the appropriate conditions for removal of the hydroxyl protecting groups on the sugar moiety (i.e., PG¹, PG², and PG³) to provide fluoride G-7. In certain embodiments, deprotection of all three protecting groups may comprise a single step. In other embodiments, deprotection of all three protecting groups may comprise more than one step. It would be apparent to one of skill in the art that any suitable protecting groups and corresponding deprotection reactions are contemplated herein.

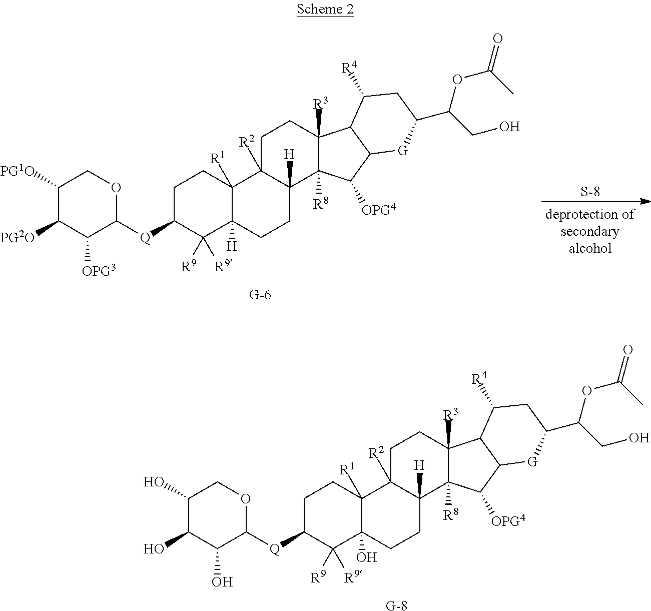

Alternatively, and as shown in step S-8 above, intermediate G-6 can be deprotected under suitable conditions to afford G-8. As discussed above, in some embodiments, deprotection of all three protecting groups may comprise a single step. In other embodiments, deprotection of all three protecting groups may comprise more than one step.

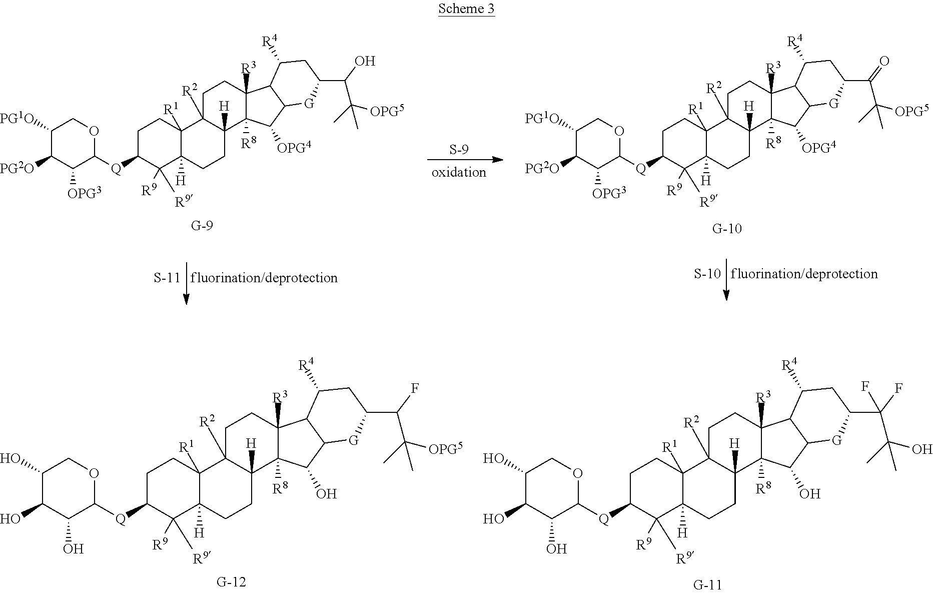

As depicted in step S-9 of Scheme 3, oxidation of G-9 produces ketone G-10. In some embodiments, oxidation occurs using a periodinane in a polar aprotic solvent capable of facilitating the oxidation. In certain embodiments, the preferred periodinane is iodosobenzoic acid in dimethylsulfoxide (DMSO).

As shown in step S-10, fluorination of G-10 via transformation of the ketone moiety into a gem-difluoro methylene unit followed by in situ deprotection of each of the alcohol moieties affords gem-difluoro polyol G-11. In some embodiments, difluoride G-11 is generated using a fluorinating agent (e.g., SF₄/HF or DAST) in an aprotic solvent (e.g., dichloromethane). Alternatively, as shown in step S-11, fluorination of G-9 can occur via displacement of the side chain alcohol followed by in situ deprotection of the alcohol moieties to furnish flourinated polyol G-12. In some embodiments, fluorination occurs as described above for step S-6 of Scheme 1.

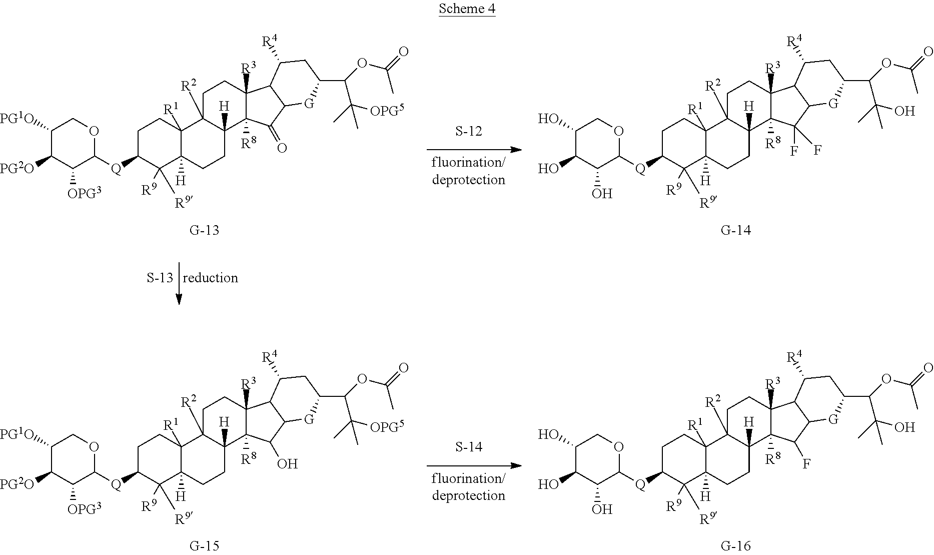

As shown in step S-12 of Scheme 4 above, fluorination of the G-13 ketone carbonyl followed by in situ deprotection provides gem-difluoro polyol G-14. Exemplary such protocols are as described above in step S-10.

As depicted in step S-13 above, G-13 can alternatively be reduced to the corresponding alcohol G-15 in preparation for subsequent fluorination via nucleophilic displacement, described above step S-6 of Scheme 1. In some embodiments, G-13 is reduced to G-15 using a suitable borohydride reducing agent such as, for instance, sodium borohydride stirred in dichloromethane.

As shown in step S-14 above, fluorination of G-15 can occur via displacement of the C-15 D ring alcohol, as described above for step S-6 of Scheme 1. In situ deprotection of the remaining protected alcohol moieties to furnish flourinated polyol G-16.

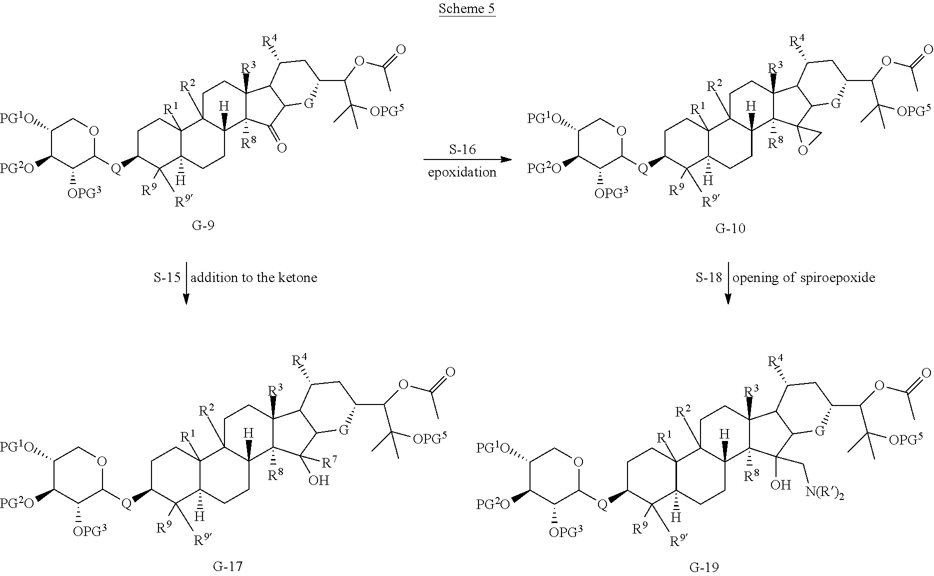

As shown in step S-15 above, addition of a suitable nucleophile to the ketone moiety of G-13 installs R⁷ and provides the corresponding alcohol G-17. In some embodiments, a polar aprotic solvent (e.g., dimethylformamide (DMF)) is used to dissolve G-13 and a solution of nucleophile is added dropwise to provide G-17. If required due to concomitant but undesired deacetylation, deacetylated G-17 can be exposed as the crude residue to acetylating conditions (e.g., acetic anhydride and DMAP as described above for step S-4 in Scheme 1).

Alternatively, and as depicted in step S-16 above, G-13 can be transformed into spiroepoxide G-18. In some embodiments, G-13 is exposed to trimethylsulfoxonium bromide in a polar aprotic solvent (e.g., DMSO) in the presence of a base, such as an alkoxide base (e.g., potassium tert-butoxide) to generate epoxide G-18.

As shown in step S-18 above, exposure of the spiroepoxide G-18 to a sufficiently basic amine opens the ring to afford amino alcohol G-19. Exemplary such amines include any amines capable of undergoing nucleophilic addition (e.g., dimethylamine, diethylamine, etc.).

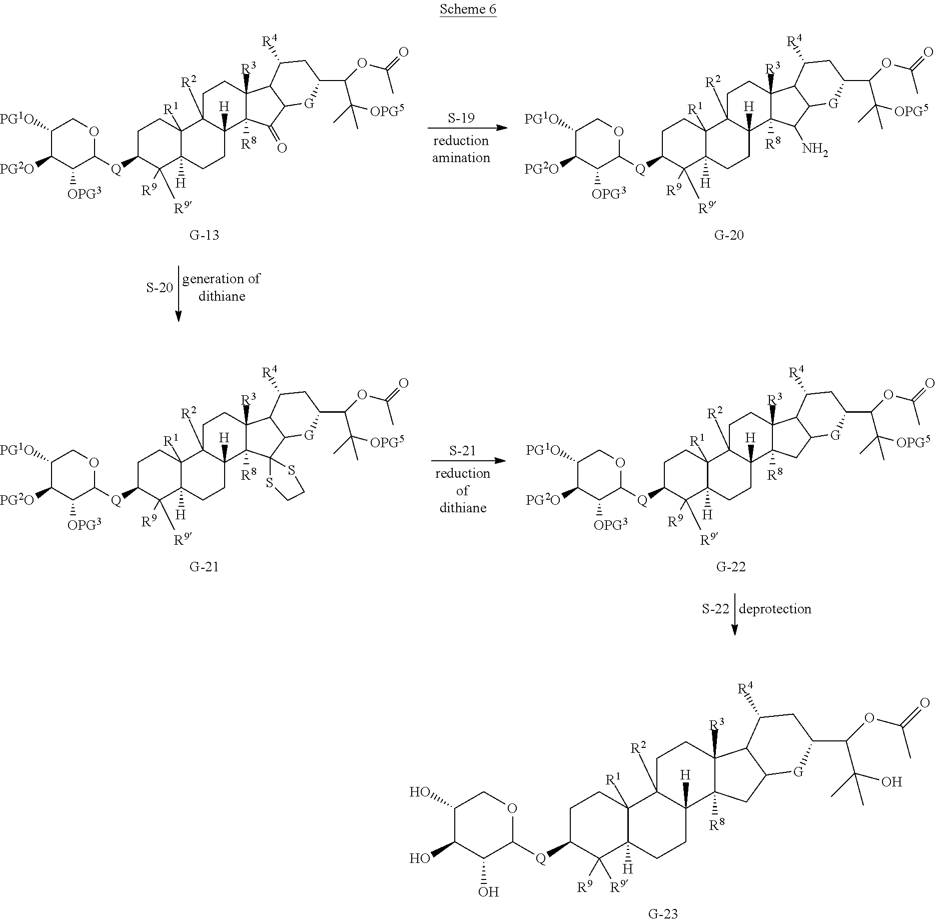

As shown in step S-19 above, reductive amination of ketone G-13 provides amine G-20. In some embodiments, a suitable amine is dissolved in an ethereal solvent (e.g., THF) in the presence of a suitable reducing agent (e.g., sodium cyanoborohydride) to furnish amine G-20.

Alternatively, and as depicted in step S-20 above, exposure of G-13 to a dithiol generates dithiane G-21. In some embodiments, a dithiol is added to G-13 under acidic conditions at reduced temperatures to furnish the desired dithiane. In certain embodiments, the acid is a Lewis acid (e.g., BF₃-Et₂O) added at temperatures of 0° C. or lower.

As shown in step S-21, dithiane G-21 can then be reduced to the corresponding methylene to afford G-22. In some embodiments, the reducing agent is Raney nickel. In some embodiments, if concomitant but undesired deacetylation occurs, the crude residue can be exposed to suitable acetylation conditions to afford acetate G-22.

As shown in step S-22, deprotection of the protected alcohol moieties of G-22 affords polyol G-23.

For each of the aforementioned Schemes, it will be readily apparent to one of ordinary skill in the art that a variety of suitable reagents and reaction conditions may be employed to carry out the described syntheses.

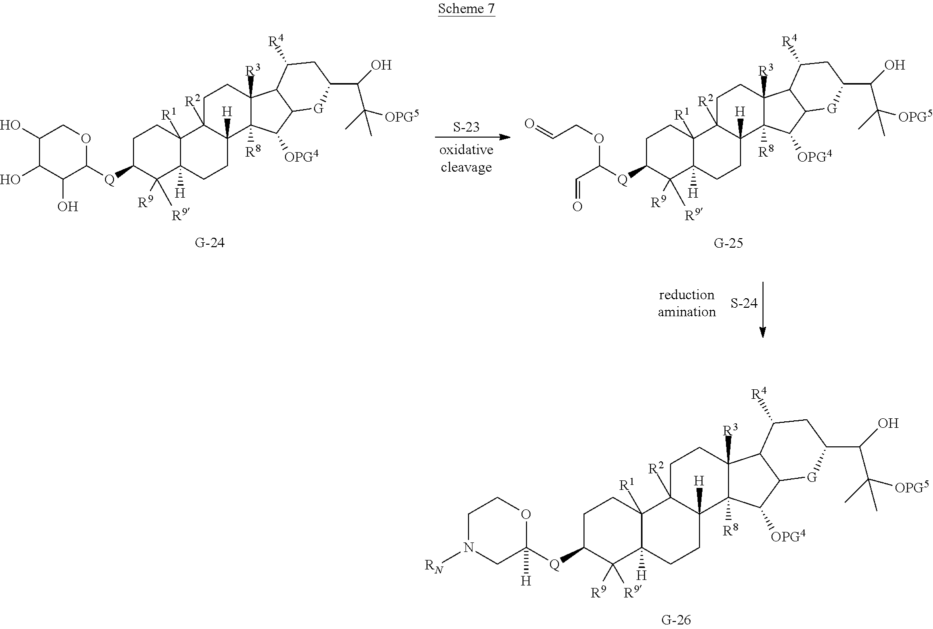

As shown in step S-23 above, oxidative cleavage of G-24 provides dialdehyde G-25. In some embodiments, a oxidative cleavage occurs in the presence of an oxidizing reagent such as a metal oxidant (e.g., Pb(OAc)₄) or a hypervalent iodide (e.g., NaIO₄). In certain embodiments, G-24 is dissolved in an alcoholic solvent (e.g., methanol) and the oxidant (e.g., NaIO₄) is added in dropwise as a solution. In certain embodiments, G-24 is dissolved in an ethereal solvent (e.g., THF) and the oxidant (e.g., NaIO₄) is added in dropwise as a solution. In some embodiments, the solution of oxidant is a solution of NaIO₄ in water. In certain embodiments, a third solvent is added to the reaction mixture. Exemplary such solvents include, but are not limited to, chlorinated solvents such as methylene chloride. Alternatively, and as mentioned above, oxidative cleavage may occur in the presence of a metal oxidant such as Pb(OAc)₄.

As shown in step S-24 above, dialdehyde G-25 can subsequently undergo a reductive amination to afford compound G-26. In some embodiments, reductive amination occurs in the presence of a primary amine or primary amine salt and an appropriate reducing agent (e.g., NaCNBH₃) in an alcoholic solvent (e.g., methanol). In some embodiments, the reaction takes from about 0.5 to about 12 hours. In some embodiments, the reaction takes from about 1 to about 9 hours. In some embodiments, the reaction takes about 3, 4, 5, 6, 7, or 8 hours.

### 14. Uses, Formulation and Administration

### Applications in Molecular Imaging: Contrast Agents

Although bones are easily visualized using x-ray imaging, many other organs and tissues cannot be easily imaged without contrast enhancement. Contrast agents, also known as contrast media or diagnostic agents, are often used during medical imaging examinations to highlight specific parts of the body (e.g tissues and organs) and make them easier to visualize and improve disease diagnosis. Contrast agents can be used with many types of imaging examinations, including ultrasound (US), x-ray exams, computed tomography scans (CT), magnetic resonance imaging (MRI), positron emission tomography (PET), and single photon emission computed tomography (SPECT) to name but a few.

As described herein, compounds of the present invention can be used to enhance the visualization of tissues and organs. Such visualization is useful for diagnosing various diseases and injuries.

In certain embodiments, the present invention provides a method for imaging one or more tissue in a patient said method comprising administering to said patient a provided compound, or composition thereof, and detecting the compound. One of ordinary skill in the art will recognize that various imaging methods are useful for the detecting step. Exemplary imaging methods are discussed further below and include x-ray, magnetic resonance, ultrasound, optical imaging, sonoluminescence, photoacoustic imaging, nuclear imaging, positron emission tomography, absorption, light scattering, and computed tomography.

In certain embodiments, the present invention provides a diagnostic imaging method comprising the steps of: (a) administering to a patient a provided compound, or composition thereof; and (b) imaging the compound after administration to the patient. In some embodiments, the present invention provides a diagnostic imaging method comprising the steps of: (a) administering to a patient a provided compound conjugated to a targeting group, or composition thereof; and (b) imaging the compound after administration to the patient.

In certain embodiments, the imaging step is selected from magnetic resonance imaging, ultrasound imaging, optical imaging, sonoluminescence imaging, photoacoustic imaging, or nuclear imaging.

In certain embodiments, the present invention provides a method of imaging one or more tissue in a patient comprising administering a provided compound, or composition thereof, and performing an imaging procedure. In some embodiments, the present invention provides a compound of Formula I containing a radioactive isotope of any suitable atom. In some embodiments, the radioactive isotope is an isotope of hydrogen, carbon, fluorine, or iodine. In certain embodiments, the isotope is selected from the group consisting of ¹¹C, ¹⁸F, ¹⁹F, ¹²³I, ¹²⁵I, and ²H.

### Ultrasound

Ultrasound is a valuable diagnostic imaging technique for studying various areas of the body including, for example, the vasculature, such as tissue microvasculature. Ultrasound provides certain advantages relative to other diagnostic techniques. For example, diagnostic techniques involving nuclear medicine and X-rays generally results in exposure of the patient to ionizing electron radiation. Such radiation can cause damage to subcellular material, including deoxyribonucleic acid (DNA), ribonucleic acid (RNA) and proteins. Ultrasound does not involve such potentially damaging radiation. In addition, ultrasound is relatively inexpensive as compared, for example, to computed tomography (CT) and magnetic resonance imaging (MRI), which require elaborate and expensive equipment.

Ultrasound involves the exposure of a patient to sound waves. Generally, the sound waves dissipate due to absorption by body tissue, penetrate through the tissue or reflect off of the tissue. The reflection of sound waves off of tissue, generally referred to as backscatter or reflectivity, forms the basis for developing an ultrasound image. In this connection, sound waves reflect differentially from different body tissues. This differential reflection is due to various factors, including, for example, the constituents and the density of the particular tissue being observed. The differentially reflected waves are detected, typically with a transducer that can detect sound waves having a frequency of one megahertz (MHz) to ten MHz. The detected waves can be integrated, quantitated and converted into an image of the tissue being studied.

Ultrasound imaging techniques typically involve the use of contrast agents to improve the quality and usefulness of images obtained. Exemplary contrast agents include, for example, suspensions of solid particles, emulsified liquid droplets, and gas-filled bubbles. See, e.g., Hilmann et al., U.S. Pat. No. 4,466,442, and published International Patent Applications WO 92/17212 and WO 92/21382.

The quality of images produced from ultrasound has improved significantly. Nevertheless, further improvement is needed, particularly with respect to images involving vasculature in tissues that are perfused with a vascular blood supply. Accordingly, there is a need for improved ultrasound techniques, including improved contrast agents, which are capable of providing medically useful images of the vasculature and vascular-related organs. In certain embodiments, the present invention provides compounds of Formula I that are useful contrast agents for ultrasound imaging techniques. In certain embodiments, said compounds are capable of providing useful images of the vasculature and vascular-related organs.

### Magnetic Resonance Imaging

MRI is in some respects it is similar to X-ray computer tomography (CT), in that it can provide (in some cases) cross-sectional images of organs with potentially excellent soft tissue resolution. In its current use, the images constitute a distribution map of protons in organs and tissues. However, unlike X-ray computer tomography, MRI does not use ionizing radiation. MRI is, therefore, a safe non-invasive technique for medical imaging.

Currently, MRI is widely used to aid in the diagnosis of many medical disorders. Examples include joint injuries, bone marrow disorders, soft tissue tumors, mediastinal invasion, lymphadenopathy, cavernous hemangioma, hemochromatosis, cirrhosis, renal cell carcinoma, uterine leiomyoma, adenomyosis, endometriosis, breast carcinomas, stenosis, coronary artery disease, aortic dissection, lipomatous hypertrophy, atrial septum, constrictive pericarditis, and the like.

Routinely employed magnetic resonance images are presently based on proton signals arising from the water molecules within cells. Consequently, it is often difficult to decipher the images and distinguish individual organs and cellular structures. There are two potential means to better differentiate proton signals. The first involves using a contrast agent that alters the T₁ or T₂ of the water molecules in one region compared to another. For example, gadolinium diethylenetriaminepentaacetic acid (Gd-DTPA) shortens the proton T₁ relaxation time of water molecules in near proximity thereto, thereby enhancing the obtained images.

Paramagnetic cations such as, for example, Gd, Mn, and Fe are excellent MRI contrast agents, as suggested above. Their ability to shorten the proton T₁ relaxation time of the surrounding water enables enhanced MRI images to be obtained which otherwise would be unreadable. The second route to differentiate individual organs and cellular structures is to introduce another nucleus for imaging (i.e., an imaging agent). Using this second approach, imaging can only occur where the contrast agent has been delivered. An advantage of this method is the fact that imaging is achieved free from interference from the surrounding water. Suitable contrast agents must be bio-compatible (i.e. non-toxic, chemically stable, not reactive with tissues) and of limited lifetime before elimination from the body.

Although hydrogen has typically been selected as the basis for MRI scanning (because of its abundance in the body) this can result in poorly imaged areas due to lack of contrast. Thus the use of other active MRI nuclei (such as fluorine) can be advantageous. The use of fluorine is advantageous because fluorine is not naturally found within the body.

A variety of specialized MRI scans have been developed for diagnostic purposes. For example, diffusion MRI measures the diffusion of water molecules in biological tissues and has enabled brain researchers to examine areas of neural degeneration and demyelination in diseases such as multiple sclerosis. Fluid Attenuated Inversion Recovery (FLAIR) is a type of specialized MRI scan used to suppress cerebrospinal fluid (CSF) so as to bring out certain types of lesions (e.g., multiple sclerosis plaques). Magnetic resonance angiography (MRA) is used to generate pictures of the arteries in order to evaluate them for stenosis (abnormal narrowing) or aneurysms. Magnetic resonance gated intracranial CSF dynamics (MR-GILD) is a method for analyzing CSF circulatory system dynamics in patients with CSF obstructuve lesions. Functional MRI (fMRI) measures signal changes in the brain due to changing neural activity.

In certain embodiments, the present invention provides compounds of Formula I that are useful contrast agents for magnetic resonance imaging techniques. In certain embodiments, said compounds are capable of providing useful images of individual organs and cellular structures. In some embodiments, provided compounds are useful in diffusion MRI techniques such as Fluid Attenuated Inversion Recovery (FLAIR). In certain embodiments, provided compounds are useful in magnetic resonance angiography (MRA) techniques. In certain embodiments, provided compounds are useful in magnetic resonance gated intracranial CSF dynamics (MR-GILD). In certain embodiments, provided compounds are useful in functional MRI techniques (fMRI).

### Positron Emission Tomography

Positron Emission Tomography (PET) is a nuclear medicine imagining technique which produces a three-dimensional image of functional processes in the body. The system detects pairs of gamma rays emitted indirectly by a positron-emitting radionuclide (tracer), which is introduced into the body on a biologically active molecule. Images of tracer concentration in 3-dimensional space within the body are then reconstructed by computer analysis. The metabolic activity observed with PET depends on the biologically active molecule administered to the subject. For instance, the fluorinated glucose analog fluorodeoxyglucose (FDG) is administered in order to image tissue metabolic activity in terms of regional glucose uptake. Other types of tracer molecules will allow imaging of other metabolic functions.

PET scans are conducted by injecting a short-lived radioactive tracer isotope into a subject. Typically, the tracer is chemically incorporated into a biologically active molecule. Once the molecule is incorporated in the tissue of interest in a sufficient concentration the subject is placed in the scanner and a record of tissue concentration is made as the tracer decays.

As mentioned above, radioisotopes used in conjunction with PET imaging, also called radionuclides, are typically isotopes with short half-lives such as carbon-11 (˜20 min) nitrogen-13 (˜10 min), oxygen-15 (˜2 min), and fluorine-18 (˜110 min). These radionuclides are incorporated either into compounds normally used by the body such as glucose or glucose analogs (e.g., FDG, described above), water, ammonia, or are incorporated into molecules that bind to receptors or other sites of drug action, called radiotracers. Thus, PET technology can be used to trace the biologic pathway of any compound in living humans provided that compound can be radiolabeled with a PET isotope. Such short-lived isotopes, while attractive because they help minimize the radiation dose received by the subject, present challenges in the manufacture of radiopharmaceuticals. In many instances, radiotracers must be produced in a radiochemistry laboratory in close proximity to the PET imaging facility.

In addition to its role as a diagnostic technique, PET has an expanding role as a method to assess the response to therapy, such as cancer therapy, where the risk to a patient from lack of knowledge about disease progression is much greater than the risk from the test radiation. PET imaging is also used for the clinical diagnosis of certain diffuse brain diseases (e.g., those causing various types of dementia) and for mapping normal human brain and heart function. PET scanning is capable of detecting areas of molecular biology detail using radiolabelled probes that have different rates of uptake depending on the type and function of tissue involved. Changing of regional blood flow as a measure of the injected positron emitter can be visualized and quantified using a PET scan.

PET scanning with the tracer fluorine-18 (F-18) fluorodeoxyglucose (FDG) is known as FDG-PET and is widely used in clinical oncology. FDG is a glucose analog that is taken up by cells and phosphorylated by hexokinase. The replacement of oxygen with fluorine prohibits metabolism of this compound and the presence of the phosphate prohibits FDG from exiting the cell. Thus, tissues with high glucose intake are intensely radiolabeled. As a result FDG-PET can be used for diagnosis, staging, and monitoring cancers.

PET scanning is also a very valuable technique for studying brain function. PET neuroimaging is based on the idea that areas of high radioactivity are associated with brain activity as indicated by glucose uptake. That is, increased blood flow to and glucose uptake in certain parts of the brain as measured using PET imaging is assumed to indicate increased activity in those parts. Conversely, brain pathologies such as Alzheimer's Disease can be screened by monitoring PET scans for areas of decreased metabolism of glucose. Several radiotracers have been developed for PET that comprise ligands for specific neuroreceptor subtypes. Examples include [¹¹C] raclopride and [¹⁸F] fallypride for dopamine D2/D3 receptors and [¹¹C] McN 5652 and [¹¹C] DASB for serotonin transporters. These tracers allow the visualization of certain neuroreceptor pools in the context of a plurality of neuropsychiatric and neurologic illnesses.

The uptake of radiolabelled drugs can also be observed using PET imaging to study biodistribution. The uptake, concentration, and elimination of a drug from a tissue can be monitored quickly and cost-effectively. Additionally, drug occupancy at a purported cite of action can be inferred using competition studies between unlabelled drugs and radiolabelled compounds thought to bind with specificity to the site.

In some embodiments, the present invention provides compounds of Formula I that are useful radiolabels and/or tracers for positron emission tomography (PET) techniques. In certain embodiments, provided compounds are capable of providing useful images of metabolic activity. In certain embodiments, provided compounds are administered in order to image tissue metabolic activity in terms of a particular chemical uptake, such as, for instance glucose uptake. In certain embodiments, provided compounds contain an isotope such as ¹¹C, ¹³N, ¹⁵O, or ¹⁸F. In certain embodiments, provided compounds contain any suitable isotope capable of being incorporated into a molecule and traced using PET techniques. In some embodiments, provided compounds may be used to monitor chemical activity in certain parts of the brain. For example, provided compounds may be used to monitor uptake, concentration, retention, and elimination of a drug. In certain embodiments, provided compounds are radiotracers developed to act as ligands for specific receptors in the brain such as, for instance, dopamine D2/D3 receptors and seratonin transporters.

### Computed Tomography

Computed tomography (CT) scanning is a medical imaging method employing tomography in order to generate a three dimensional image of the inside of an object from a large series of two dimensional X-ray images taken around a single axis of rotation. Tomography can be performed by moving the X-ray source and detector during an exposure, causing the anatomy at the target level to remain sharp, while structures at different levels are blurred. By varying the extent and path of motion, a variety of effects can be obtained, with variable depth of field and different degrees of blurring of ‘out of plane’ structures.

CT scanning of the head is typically used to detect bleeding, brain injury and skull fractures, bleeding due to a ruptured/leaking aneurysm in a patient with a sudden severe headache, a blood clot or bleeding within the brain shortly after a patient exhibits symptoms of a stroke, strokes, brain tumors, enlarged brain cavities in patients with hydrocephalus, diseases/malformations of the skull, bone and soft tissue damage in patients with facial trauma, diseases of the temporal bone on the side of the skull, which may be causing hearing problems, or inflammation or other changes present in the paranasal sinuses. CT scanning may also be used to plan radiation therapy for cancer of the brain or other tissues, guide the passage of a needle used to obtain a tissue sample (biopsy) from the brain, or assess aneurysms or arteriovenous malformations.

CT can be used for detecting both acute and chronic changes in the lung parenchyma, that is, the internals of the lungs. For detection of airspace disease (such as pneumonia) or cancer, relatively thick sections and general purpose image reconstruction techniques may be adequate. IV contrast may also be used as it clarifies the anatomy and boundaries of the vasculature.

CT angiography of the chest is also becoming the primary method for detecting pulmonary embolism (PE) and aortic dissection, and requires accurately timed rapid injections of contrast (Bolus Tracking) and high-speed helical scanners. CT is the standard method of evaluating abnormalities seen on chest X-ray and of following findings of uncertain acute significance. A CT pulmonary angiogram (CTPA) is a medical diagnostic test used to diagnose pulmonary embolism (PE). It employs computed tomography to obtain an image of the pulmonary arteries.

With the advent of subsecond rotation combined with multi-slice CT (up to 64-slice), high resolution and high speed can be obtained at the same time, allowing excellent imaging of the coronary arteries (cardiac CT angiography). Images with an even higher temporal resolution can be formed using retrospective ECG gating. In this technique, each portion of the heart is imaged more than once while an ECG trace is recorded. The ECG is then used to correlate the CT data with their corresponding phases of cardiac contraction. Once this correlation is complete, all data that were recorded while the heart was in motion (systole) can be ignored and images can be made from the remaining data that happened to be acquired while the heart was at rest (diastole). In this way, individual frames in a cardiac CT investigation have a better temporal resolution than the shortest tube rotation time.

CT is a sensitive method for diagnosis of abdominal diseases. It is used frequently to determine stage of cancer and to follow progress. It is also a useful test to investigate acute abdominal pain (especially of the lower quadrants, whereas ultrasound is the preferred first line investigation for right upper quadrant pain). Renal stones, appendicitis, pancreatitis, diverticulitis, abdominal aortic aneurysm, and bowel obstruction are conditions that are readily diagnosed and assessed with CT.

Oral and/or rectal contrast may be used depending on the indications for the scan. A dilute (2% w/v) suspension of barium sulfate is most commonly used. The concentrated barium sulfate preparations used for fluoroscopy e.g. barium enema are too dense and cause severe artifacts on CT. Iodinated contrast agents may be used if barium is contraindicated (for example, suspicion of bowel injury). Other agents may be required to optimize the imaging of specific organs, such as rectally administered gas (air or carbon dioxide) or fluid (water) for a colon study, or oral water for a stomach study.

CT is also used in osteoporosis studies and research alongside dual energy X-ray absorptiometry (DXA). Both CT and DXA can be used to assess bone mineral density (BMD) which is used to indicate bone strength, however CT results do not correlate exactly with DXA (the gold standard of BMD measurement). CT is far more expensive, and subjects patients to much higher levels of ionizing radiation, so it is used infrequently. CT is often used to image complex fractures, especially ones around joints, because of its ability to reconstruct the area of interest in multiple planes.

As mentioned above, in certain instances it is desirable to use a contrast agent when obtaining a CT scan. Contrast agents, also referred to as “dyes”, are used to highlight specific areas so that the organs, blood vessels, or tissues are more visible. Common contrast agents include iodine, barium, barium sufate, and gastrografin and may be administered via intravenous injection, oral administration, rectal administration, or in the case of xenon gas, via inhalation.

In some embodiments, the present invention provides compounds of Formula I that are useful contrast agents for CT scanning techniques. In certain embodiments, said provided compounds act as dyes similar, for instance, to iodine or barium as discussed above.

Provided compounds useful as imaging agents may be formulated and administered using any of the methods described herein and below.

### 15. Pharmaceutically Acceptable Compositions

According to another aspect of the present invention, pharmaceutically acceptable compositions are provided, wherein these compositions comprise any of the compounds as described herein, and optionally comprise a pharmaceutically acceptable carrier, adjuvant or vehicle. In certain embodiments, these compositions optionally further comprise one or more additional therapeutic agents.

It will also be appreciated that certain of the compounds of present invention can exist in free form for treatment, or where appropriate, as a pharmaceutically acceptable salt thereof.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. A “pharmaceutically acceptable salt” means any non-toxic salt or salt of an ester of a compound of this invention that, upon administration to a recipient, is capable of providing, either directly or indirectly, a compound of this invention or a pharmaceutically active metabolite or residue thereof. As used herein, the term “pharmaceutically active metabolite or residue thereof” means that a metabolite or residue thereof is also a pharmaceutically active compound in accordance with the present invention.

Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N⁺(C₁₋₄ alkyl)₄ salts. This invention also envisions the quaternization of any basic nitrogen-containing groups of the compounds disclosed herein. Water or oil-soluble or dispersable products may be obtained by such quaternization. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

In some cases, compounds of the present invention may contain one or more acidic functional groups and, thus, may be capable of forming pharmaceutically-acceptable salts with pharmaceutically-acceptable bases. The term “pharmaceutically-acceptable salts” in these instances refers to the relatively non-toxic, inorganic and organic base addition salts of compounds of the present invention. These salts can likewise be prepared in situ in the administration vehicle or the dosage form manufacturing process, or by separately reacting the purified compound in its free acid form with a suitable base, such as the hydroxide, carbonate or bicarbonate of a pharmaceutically-acceptable metal cation, with ammonia, or with a pharmaceutically-acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like. See, for example, Berge et al., supra.

The compositions of the present invention may additionally comprise a pharmaceutically acceptable carrier, adjuvant, or vehicle, which, as used herein, includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical Sciences, Sixteenth Edition, E. W. Martin (Mack Publishing Co., Easton, Pa., 1980) discloses various carriers used in formulating pharmaceutically acceptable compositions and known techniques for the preparation thereof. Except insofar as any conventional carrier medium is incompatible with the compounds of the invention, such as by producing any undesirable biological effect or otherwise interacting in a deleterious manner with any other component(s) of the pharmaceutically acceptable composition, its use is contemplated to be within the scope of this invention. Some examples of materials which can serve as pharmaceutically acceptable carriers include, but are not limited to, ion exchangers, alumina, aluminum stearate, lecithin, serum proteins, such as human serum albumin, buffer substances such as phosphates, glycine, sorbic acid, or potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, polyacrylates, waxes, polyethylene-polyoxypropylene-block polymers, wool fat, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such a propylene glycol or polyethylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

The compositions provided by the present invention can be employed in combination therapies, meaning that the present compositions can be administered concurrently with, prior to, or subsequent to, one or more other desired therapeutic agents or medical procedures. The particular combination of therapies (therapeutic agents or procedures) to employ in a combination regimen will take into account compatibility of the desired therapeutic agents and/or procedures and the desired therapeutic effect to be achieved. It will also be appreciated that the therapies employed may achieve a desired effect for the same disorder (for example, a compound described herein may be administered concurrently with another therapeutic agent used to treat the same disorder), or they may achieve different effects (e.g., control of any adverse effects).

For example, known agents useful for treating neurodegenerative disorders may be combined with the compositions of this invention to treat neurodegenerative disorders, such as Alzheimer's disease. Examples of such known agents useful for treating neurodegenerative disorders include, but are not limited to, treatments for Alzheimer's disease such as acetylcholinesterase inhibitors, including donepezil, Exelon® and others; memantine (and related compounds as NMDA inhibitors), treatments for Parkinson's disease such as L-DOPA/carbidopa, entacapone, ropinrole, pramipexole, bromocriptine, pergolide, trihexephendyl, and amantadine; agents for treating Multiple Sclerosis (MS) such as beta interferon (e.g., Avonex® and Rebif®), Copaxone®, and mitoxantrone; riluzole, and anti-Parkinsonian agents. For a more comprehensive discussion of updated therapies useful for treating neurodegenerative disorders, see, a list of the FDA approved drugs at http://www.fda.gov, and The Merck Manual, Seventeenth Ed. 1999, the entire contents of which are hereby incorporated by reference.

Additional examples of such known agents useful for treating neurodegenerative disorders include, but are not limited to, beta-secretase inhibitors/modulators, gamma-secretase inhibitors/modulators, HMG-CoA reductase inhibitors, NSAID's including ibuprofen, vitamin E, anti-amyloid antibodies, including humanized monoclonal antibodies, inhibitors/modulators of tau phosphorylation (such as GSK3 or CDK inhibitors/modulators) and/or aggregation, CB-1 receptor antagonists or CB-1 receptor inverse agonists, antibiotics such as doxycycline and rifampin, N-methyl-D-aspartate (NMDA) receptor antagonists, such as mematine, cholinesterase inhibitors such as galantamine, rivastigmnine, donepezil and tacrine, growth hormone secretagogues such as ibutamoren, ibutamoren mesylate and capromorelin, histamine H₃ antagonists, AMPA agonists, PDE-IV, -V, -VII, -VIII, and -IX inhibitors, GABA_{A} inverse agonists, and neuronal nicotinic agonists and partial agonists, serotonin receptor antagonists.

In certain embodiments, an additional therapeutic agent is selected from acetylcholinesterase inhibitors, NMDA inhibitors, agents for treating multiple sclerosis, anti-Parkinsonian agents, beta-secretase inhibitors/modulators, gamma-secretase inhibitors/modulators, HMG-CoA reductase inhibitors, NSAID's, anti-amyloid antibodies, including humanized monoclonal antibodies, CB-1 receptor antagonists or CB-1 receptor inverse agonists, antibiotics, cholinesterase inhibitors, growth hormone secretagogues, histamine H₃ antagonists, AMPA agonists, PDE inhibitors, including selective and mixed inhibitors of -II, -IV, -V, -VII, -VIII, -IX and X inhibitors, GABA_{A} inverse agonists, neuronal nicotinic agonists and partial agonists, serotonin receptor antagonists, inhibitors/modulators of tau phosphorylation and/or aggregation, GSK3 inhibitors/modulators, CDK inhibitors/modulators, N-methyl-D-aspartate (NMDA) receptor antagonists, m-TOR inhibitors, metal chelators, antioxidants, neuroprotectants, Exelon®, memantine, L-DOPA/carbidopa, entacapone, ropinrole, pramipexole, bromocriptine, pergolide, trihexephendyl, amantadine, beta interferon (e.g., Avonex® and Rebif®), Copaxone®, and mitoxantrone; riluzole, ibuprofen, vitamin E, doxycycline and rifampine, galantamine, rivastigmnine, donepezil and tacrine, ibutamoren, ibutamoren mesylate and capromorelin.

In other embodiments, the compounds of the present invention are combined with other agents useful for treating neurodegenerative disorders, such as Alzheimer's disease, wherein such agents include beta-secretase inhibitors/modulators, gamma-secretase inhibitors/modulators, anti-amyloid antibodies, including humanized monoclonal antibodies aggregation inhibitors, metal chelators, antioxidants, and neuroprotectants and inhibitors/modulators of tau phosphorylation (such as GSK3 or CDK inhibitors/modulators) and/or aggregation.

In some embodiments, compounds of the present invention are combined with gamma secretase modulators. In some embodiments, compounds of the present invention are gamma secretase modulators combined with gamma secretase modulators. Exemplary such gamma secretase modulators include, inter alia, certain NSAIDs and their analogs (see WO01/78721 and US 2002/0128319 and Weggen et al., Nature, 414 (2001) 212-16; Morihara et al., J. Neurochem., 83 (2002), 1009-12; and Takahashi et al., J. Biol. Chem., 278 (2003), 18644-70).

As used herein, the term “combination,” “combined,” and related terms refers to the simultaneous or sequential administration of therapeutic agents in accordance with this invention. For example, a compound of the present invention may be administered with another therapeutic agent simultaneously or sequentially in separate unit dosage forms or together in a single unit dosage form. Accordingly, the present invention provides a single unit dosage form comprising a provided compound, an additional therapeutic agent, and a pharmaceutically acceptable carrier, adjuvant, or vehicle.

Other examples of agents the compounds of this invention may also be combined with include, without limitation: treatments for asthma such as albuterol and Singulair®; agents for treating schizophrenia such as zyprexa, risperdal, seroquel, and haloperidol; anti-inflammatory agents such as corticosteroids, TNF blockers, IL-1 RA, azathioprine, cyclophosphamide, and sulfasalazine; immunomodulatory and immunosuppressive agents such as cyclosporin, tacrolimus, rapamycin, mycophenolate mofetil, interferons, corticosteroids, cyclophosphamide, azathioprine, and sulfasalazine; neurotrophic factors such as acetylcholinesterase inhibitors, MAO inhibitors, interferons, anti-convulsants, ion channel blockers, agents for treating cardiovascular disease such as beta-blockers, ACE inhibitors, diuretics, nitrates, calcium channel blockers, and statins; agents for treating liver disease such as corticosteroids, cholestyramine, interferons, and anti-viral agents; agents for treating blood disorders such as corticosteroids, anti-leukemic agents, and growth factors; and agents for treating immunodeficiency disorders such as gamma globulin.

The amount of additional therapeutic agent present in the compositions of this invention will be no more than the amount that would normally be administered in a composition comprising that therapeutic agent as the only active agent. In certain embodiments, the amount of additional therapeutic agent in the present compositions will range from about 50% to 100% of the amount normally present in a composition comprising that agent as the only therapeutically active agent.

In an alternate embodiment, the methods of this invention that utilize compositions that do not contain an additional therapeutic agent, comprise the additional step of separately administering to said patient an additional therapeutic agent. When these additional therapeutic agents are administered separately they may be administered to the patient prior to, sequentially with or following administration of the compositions of this invention.

The pharmaceutically acceptable compositions of this invention can be administered to humans and other animals orally, rectally, parenterally, intracisternally, intravaginally, intraperitoneally, topically (as by powders, ointments, or drops), bucally, as an oral or nasal spray, or the like, depending on the severity of the disorder being treated. In certain embodiments, the compounds of the invention may be administered orally or parenterally at dosage levels of about 0.01 mg/kg to about 50 mg/kg and preferably from about 1 mg/kg to about 25 mg/kg, of subject body weight per day, one or more times a day, to obtain the desired therapeutic effect.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound of the present invention, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with one or more inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with one or more inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of this invention. Additionally, the present invention contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

In some embodiments, the present invention provides a composition containing a provided compound in an amount of about 1 weight percent to about 99 weight percent. In other embodiments, the composition contains a provided compound wherein the composition contains no more than about 10.0 area percent HPLC of other components of black cohosh root relative to the total area of the HPLC chromatogram. In other embodiments, the composition containing a provided compound contains no more than about 8.0 area percent HPLC of other components of black cohosh root relative to the total area of the HPLC chromatogram, and in still other embodiments, no more than about 3 area percent.

### 16. Uses of Compounds and Pharmaceutically Acceptable Compositions

Alzheimer's Disease (AD) is believed to result from the deposition of quantities of a peptide, amyloid-beta (“A-beta”), within the brain. This peptide is produced by enzymatic cleavage of amyloid protein precursor (“APP”) protein. The C-terminus of A-beta is generated by an enzyme termed gamma-secretase. Cleavage occurs at more than one site on APP producing different length A-beta peptides, some of which are more prone to deposition, such as A-beta 42. It is believed that aberrant production A-beta 42 in the brain leads to AD.

A-beta, a 37-43 amino acid peptide derived by proteolytic cleavage of the amyloid precursor protein (APP), is the major component of amyloid plaques. APP is expressed and constitutively catabolized in most cells. APP has a short half-life and is metabolized rapidly down two pathways. In one pathway, cleavage by an enzyme known as alpha-secretase occurs while APP is still in the trans-Golgi secretory compartment. This cleavage by alpha-secretase occurs within the A-beta portion of APP, thus precluding the formation of A-beta.

In contrast to this non-amyloidogenic pathway involving alpha-secretase described above, proteolytic processing of APP by beta-secretase exposes the N-terminus of A-beta, which after gamma-secretase cleavage at the variable C-terminus, liberates A-beta. Peptides of 40 or 42 amino acids in length (A-beta 1-40 and A-beta 1-42, respectively) predominate among the C-termini generated by gamma-secretase, however, a recent report suggests 1-38 is a dominant species in cerebrospinal fluid. A-beta 1-42 is more prone to aggregation than A-beta 1-40, the major component of amyloid plaque, and its production is closely associated with the development of Alzheimer's disease. The bond cleaved by gamma-secretase appears to be situated within the transmembrane domain of APP. In the amyloidogenic pathway, APP is cleaved by beta-secretase to liberate sAPP-beta and CTF-beta, which CTF-beta is then cleaved by gamma-secretase to liberate the harmful A-beta peptide.

While abundant evidence suggests that extracellular accumulation and deposition of A-beta is a central event in the etiology of AD, recent studies have also proposed that increased intracellular accumulation of A-beta or amyloid containing C-terminal fragments may play a role in the pathophysiology of AD. For example, over-expression of APP harboring mutations which cause familial Alzheimer's disease (AD) results in the increased intracellular accumulation of CTF-beta in neuronal cultures and A-beta 42 in HEK 293 cells.

A-beta 42 is the 42 amino acid long form of A-beta that is believed to be more potent in forming amyloid plaques than the shorter forms of A-beta. Moreover, evidence suggests that intra- and extracellular A-beta are formed in distinct cellular pools in hippocampal neurons and that a common feature associated with two types of familial AD mutations in APP (“Swedish” and “London”) is an increased intracellular accumulation of A-beta 42.

Without wishing to be bound by theory, it is believed that of importance in this A-beta-producing pathway is the position of the gamma-secretase cleavage. If the gamma-secretase proteolytic cut is at residue or before 711-712, shorter A-beta. (A-beta 40 or shorter) is the result; if it is a proteolytic cut after residue 713, long A-beta (A-beta 42) is the result. Thus, the .gamma. secretase process is central to the production of A-beta peptide of 40 or 42 amino acids in length (A-beta 40 and A-beta 42, respectively). For a review that discusses APP and its processing, see Selkoe, 1998, Trends Cell. Biol. 8:447-453; Selkoe, 1994, Ann. Rev. Cell Biol. 10:373-403. See also, Esch et al., 1994, Science 248:1122.

Cleavage of APP can be detected in a number of convenient manners, including the detection of polypeptide or peptide fragments produced by proteolysis. Such fragments can be detected by any convenient means, such as by antibody binding. Another convenient method for detecting proteolytic cleavage is through the use of a chromogenic .beta. secretase substrate whereby cleavage of the substrate releases a chromogen, e.g., a colored or fluorescent, product. More detailed analyses can be performed including mass spectroscopy.

Much interest has focused on the possibility of inhibiting the development of amyloid plaques as a means of preventing or ameliorating the symptoms of Alzheimer's disease. To that end, a promising strategy is to inhibit the activity of beta- and/or gamma-secretase, the two enzymes that together are responsible for producing A-beta. This strategy is attractive because, if amyloid plaque formation as a result of A-beta deposition is a cause of Alzheimer's disease, then inhibiting the activity of one or both of the two secretases would intervene in the disease process at an early stage, before late-stage events such as inflammation or apoptosis occur.

Modulators of gamma-secretase may function in a variety of ways. They may block gamma-secretase completely, or they may alter the activity of the enzyme so that less A-beta 42 and more of the alternative, soluble, forms of A-beta, such as A-beta 37, 38 or 39 are produced. Such modulators may thereby retard or reverse the development of AD.

Compounds are known, such as indomethacin, ibuprofen and sulindac sulphide, which inhibit the production of A-beta 42 while increasing the production of A-beta 38 and leaving the production of A-beta 40 constant.

In some embodiments, compounds of the present invention are useful gamma-secretase modulators. In some embodiments, compounds of the present invention modulate the action of gamma-secretase such that amyloid-beta (1-42) peptide production in a patient is attenuated. In certain embodiments, compounds of the present invention modulate the action of gamma-secretase so as to selectively attentuate amyloid-beta (1-42) peptide production in a patient. In some embodiments, such selective attenuation occurs without significantly lowering production of the total pool of Abeta, or the specific shorter chain isoformamyloid-beta (1-40) peptide. In some embodiments, such selective attenuation results in secretion of amyloid beta which has less tendency to self-aggregate and form insoluble deposits, is more easily cleared from the brain, and/or is less neurotoxic. In some embodiments, the ability of compounds of the present invention to modulate gamma-secretase is beneficial in that there is a reduced risk of side effects with treatment resulting from, e.g., minimal disruption of other gamma-secretase controlled signaling pathways.

In some embodiments, compounds of the present invention are gamma-secretase modulators useful for treating a patient suffering from AD, cerebral amyloid angiopathy, HCHWA-D, multi-infarct dementia, dementia pugilistica or traumatic brain injury and/or Down syndrome.

In some embodiments, one or more compounds of the present invention are administered to a patient suffering from mild cognitive impairment or age-related cognitive decline or pre-symptomatic AD or prodromal or predementia AD (Dubois et al The Lancet Neurology 10 (2010) 70223-4 A favourable outcome of such treatment is prevention or delay of the onset of AD. Age related cognitive decline and mild cognitive impairment (MCI) are conditions in which a memory deficit is present, but other diagnostic criteria for dementia are absent (Santacruz and Swagerty, American Family Physician, 63 (2001), 703-13). As used herein, “age-related cognitive decline” implies a decline of at least six months' duration in at least one of: memory and learning; attention and concentration; thinking; language; and visuospatial functioning and a score of more than one standard deviation below the norm on standardized neuropsychologic testing such as the MMSE.

In some embodiments, compounds of the present invention are useful for modulating and/or inhibiting amyloid-beta (1-42) peptide production in a patient. Accordingly, compounds of the present invention are useful for treating, or lessening the severity of, disorders associated with amyloid-beta (1-42) peptide production in a patient.

In some embodiments, the compounds of the present invention are useful for modulating and/or inhibiting amyloid-beta (1-40) peptide production in a patient. Accordingly, the compounds of the present invention are useful for treating, or lessening the severity of, disorders associated with amyloid-beta (1-40) peptide production in a patient. In some embodiments, compounds of the present invention do not modulate and/or inhibit amyloid-beta (1-40) peptide production in a patient.

In some embodiments, the compounds of the present invention are useful for modulating and/or inhibiting amyloid-beta (1-38) peptide production in a patient. Accordingly, the compounds of the present invention are useful for treating, or lessening the severity of, disorders associated with amyloid-beta (1-38) peptide production in a patient.

In some embodiments, the compounds of the present invention are useful for reducing both amyloid-beta (1-42) and amyloid beta (1-38). In some embodiments, the compounds of the present invention are useful for reducing amyloid-beta (1-42) and raising amyloid beta (1-38).

The compounds, extracts, and compositions, according to the method of the present invention, may be administered using any amount and any route of administration effective for treating or lessening the severity of a neurodegenerative disorder. The exact amount required will vary from subject to subject, depending on the species, age, and general condition of the subject, the severity of the infection, the particular agent, its mode of administration, and the like.

In certain embodiments, the present invention provides a method for modulating and/or inhibiting amyloid-beta (1-42) peptide production in a patient, wherein said method comprises administering to said patient a provided compound, or a pharmaceutically acceptable composition comprising said compound. In other embodiments, the present invention provides a method of selectively modulating and/or inhibiting amyloid-beta (1-42) peptide production in a patient, wherein said method comprises administering to said patient a provided compound, or a pharmaceutically acceptable composition thereof. In still other embodiments, the present invention provides a method of reducing amyloid-beta (1-42) peptide levels in a patient, wherein said method comprises administering to said patient a provided compound, or a pharmaceutically acceptable composition thereof. In other embodiments, the present invention provides a method for reducing amyloid-beta (1-42) peptide levels in a cell, comprising contacting said cell with a provided compound. Another embodiment provides a method for reducing amyloid-beta (1-42) in a cell without substantially reducing amyloid-beta (1-40) peptide levels in the cell, comprising contacting said cell with a provided compound. Yet another embodiment provides a method for reducing amyloid-beta (1-42) in a cell and increasing one or more of amyloid-beta (1-37) and amyloid-beta (1-39) in the cell, comprising contacting said cell with a provided compound.

As used herein, the term “reducing” or “reduce” refers to the relative decrease in the amount of an amyloid-beta achieved by administering a provided compound as compared to the amount of that amyloid-beta in the absence of administering a provided compound. By way of example, a reduction of amyloid-beta (1-42) means that the amount of amyloid-beta (1-42) in the presence of a provided compound is lower than the amount of amyloid-beta (1-42) in the absence of a provided compound.

In still other embodiments, the present invention provides a method for selectively reducing amyloid-beta (1-42) peptide levels in a patient, wherein said method comprises administering to said patient a provided compound, or a pharmaceutically acceptable composition thereof. In certain embodiments, the present invention provides a method for reducing amyloid-beta (1-42) peptide levels in a patient without substantially reducing amyloid-beta (1-40) peptide levels, wherein said method comprises administering to said patient a provided compound, or a pharmaceutically acceptable composition thereof.

In certain embodiments, the present invention provides a method for reducing amyloid-beta (1-42) peptide levels in a patient and increasing one or more of amyloid-beta (1-37) and amyloid-beta (1-39), wherein said method comprises administering to said patient a provided compound, or a pharmaceutically acceptable composition thereof.

In certain embodiments, the present invention provides a method for reducing amyloid-beta (1-42) peptide levels in a patient and increasing amyloid-beta (1-38), wherein said method comprises administering to said patient a provided compound, or a pharmaceutically acceptable composition thereof. In certain embodiments, the present invention provides a method for reducing amyloid-beta (1-42) peptide levels in a patient and decreasing amyloid-beta (1-38), wherein said method comprises administering to said patient a provided compound, or a pharmaceutically acceptable composition thereof.

The term “increasing” or “increase,” as used herein in reference to an amount of an amyloid-beta, refers to the relative rise in the amount of an amyloid-beta achieved by administering a provided compound (or contacting a cell with a provided compound) as compared to the amount of that amyloid-beta in the absence of administering a provided compound (or contacting a cell with a provided compound). By way of example, an increase of amyloid-beta (1-37) means that the amount of amyloid-beta (1-37) in the presence of a provided compound is higher than the amount of amyloid-beta (1-37) in the absence of a provided compound. For instance, the relative amounts of either of amyloid-beta (1-37) and amyloid-beta (1-39) can be increased either by an increased production of either of amyloid-beta (1-37) and amyloid-beta (1-39) or by a decreased production of longer amyloid-beta peptides, e.g., amyloid-beta (1-40) and/or amyloid-beta (1-42). In addition, it will be appreciated that the term “increasing” or “increase,” as used herein in reference to an amount of an amyloid-beta, refers to the absolute rise in the amount of an amyloid-beta achieved by administering a provided compound. Thus, in certain embodiments, the present invention provides a method for increasing the absolute level of one or more of amyloid-beta (1-37) and amyloid-beta (1-39), wherein said method comprises administering to said patient a provided compound, or a pharmaceutically acceptable composition thereof. In other embodiments, the present invention provides a method for increasing the level of one or more of amyloid-beta (1-37) and amyloid-beta (1-39), wherein the increase is relative to the amount of longer amyloid-beta peptides, e.g., amyloid-beta (1-40) and/or amyloid-beta (1-42), or total amyloid-beta, wherein said method comprises administering to said patient a provided compound, or a pharmaceutically acceptable composition thereof.

One of ordinary skill in the art will appreciate that overall ratio of amyloid-beta peptides is significant where selective reduction of amyloid-beta (1-42) is especially advantageous. In certain embodiments, the present compounds reduce the overall ratio of amyloid-beta (1-42) peptide to amyloid-beta (1-40) peptide. Accordingly, another aspect of the present invention provides a method for reducing the ratio of amyloid-beta (1-42) peptide to amyloid-beta (1-40) peptide in a patient, comprising administering to said patient a provided compound, or a pharmaceutically acceptable composition thereof. In certain embodiments, the ratio of amyloid-beta (1-42) peptide to amyloid-beta (1-40) peptide is reduced from a range of about 0.1 to about 0.4 to a range of about 0.05 to about 0.08.

In other embodiments, the present invention provides a method for reducing the ratio of amyloid-beta (1-42) peptide to amyloid-beta (1-40) peptide in a cell, comprising contacting the cell with a provided compound. In certain embodiments, the ratio of amyloid-beta (1-42) peptide to amyloid-beta (1-40) peptide is reduced from a range of about 0.1 to about 0.4 to a range of about 0.05 to about 0.08.

According to one aspect, the present invention provides a method for treating or lessening the severity of a disorder associated with amyloid-beta (1-42) peptide, wherein said method comprises administering to said patient a provided compound, or a pharmaceutically acceptable composition thereof. Such disorders include neurodegenerative disorders such as Alzheimer's disease, Parkinson's disease, and Down's syndrome.

Such disorders also include inclusion body myositis (deposition of A-beta in peripheral muscle, resulting in peripheral neuropathy), cerebral amyloid angiopathy (amyloid in the blood vessels in the brain), and mild cognitive impairment and pre-symptomatic, prodromal or predementia AD.

“High A-beta42” is a measurable condition that precedes symptomatic disease, especially in familial patients, based on plasma, CSF measurements, and/or genetic screening or brain imaging. This concept is analogous to the relationship between elevated cholesterol and heart disease. Thus, another aspect of the present invention provides a method for preventing a disorder associated with elevated amyloid-beta (1-42) peptide, wherein said method comprises administering to said patient a provided compound or a pharmaceutically acceptable composition thereof.

In other embodiments, the present invention provides a method for treating diseases where A-beta amyloidosis may be an underlying aspect or a co-existing and exacerbating factor, wherein said method comprises administering to said patient a provided compound, or a pharmaceutically acceptable composition thereof.

In still other embodiments, the present invention provides a method for treating a disorder in a patient, wherein said method comprises administering to said patient a provided compound, or a pharmaceutically acceptable composition thereof, and wherein said disorder is Lewy body dementia (associated with deposition of alpha-synuclein into Lewy bodies in cognitive neurons; a-synuclein is more commonly associated with deposits in motor neurons and the etiology of Parkinson's disease), Parkinson's disease, cataract (where a-beta is aggregating in the eye lens), age-related macular degeneration, Tauopathies (e.g. frontotemporal dementia), Huntington's disease, ALS/Lou Gerhig's disease, Type 2 diabetes (IAPP aggregates in pancreatic islets, is similar in size and sequence to A-beta and having type 2 diabetes increases risk of dementia), transthyretin amyloid disease (TTR, an example of this disease is in heart muscle contributing to cardiomyopathy), prion disease (including Creutzfeldt-Jakob disease, Gerstmann-Sträussler-Scheinker syndrome, fatal familial insomnia, and kuru), and CJD.

In some embodiments, the present invention provides a method for treating a disorder in a patient, wherein said method comprises administering to said patient a provided compound, or a pharmaceutically acceptable composition thereof, and wherein said disorder is mild cognitive impairment, pre-symptomatic AD, prodromal or predementia AD, Trisomy 21 (Down Syndrome), cerebral amyloid angiopathy, degenerative dementia, Hereditary Cerebral Hemorrhage with Amyloidosis of the Dutch-Type (HCHWA-D), Creutzfeld-Jakob disease, prion disorders, amyotrophic lateral sclerosis, progressive supranuclear palsy, head trauma, stroke, Down syndrome, pancreatitis, inclusion body myositis, other peripheral amyloidoses, diabetes and atherosclerosis, cerebral amyloid angiopathy, HCHWA-D, multi-infarct dementia, and/or dementia pugilistica, or traumatic brain injury.

In other embodiments, the present invention provides a method for treating or lessening the severity of Alzheimer's disease in a patient, wherein said method comprises administering to said patient a provided compound, or a pharmaceutically acceptable composition thereof.

Without wishing to be bound by any particular theory, it is believed that the present compounds are modulators of gamma-secretase which selectively reduce levels of amyloid-beta (1-42). Accordingly, another embodiment of the present invention provides a method of modulating gamma-secretase in a patient, comprising administering to said patient a provided compound, or pharmaceutically acceptable composition thereof. In certain embodiments, the present compounds are inhibitors of gamma-secretase. Said method is useful for treating or lessening the severity of any disorder associated with gamma-secretase. Such disorders include, without limitation, neurodegenerative disorders, e.g. Alzheimer's disease. In some embodiments, such disorders include cerebral amyloid angiopathy, HCHWA-D, multi-infarct dementia, dementia pugilistica, traumatic brain injury and/or Down syndrome.

The Notch/Delta signaling pathway is highly conserved across species and is widely used during both vertebrate and invertebrate development to regulate cell fate in the developing embryo. See Gaiano and Fishell, “The Role of Notch in Promoting Glial and Neural Stem Cell Fates” Annu. Rev. Neurosci. 2002, 25:471-90. Notch interacts with the gamma-secretase complex and has interactions with a variety of other proteins and signaling pathways. Notchl competes with the amyloid precursor protein for gamma-secretase and activation of the Notch signaling pathway down-regulates PS-1 gene expression. See Lleo et al, “Notchl Competes with the Amyloid Precursor Protein for γ-Secretase and Down-regulates Presenilin-1 Gene Expression” Journal of Biological Chemistry 2003, 48:47370-47375. Notch receptors are processed by gamma-secretase acting in synergy with T cell receptor signaling and thereby sustain peripheral T cell activation. Notchl can directly regulate Tbx21 through complexes formed on the Tbx21 promoter. See Minter et al., “Inhibitors of γ-secretase block in vivo and in vitro T helper type 1 polarization by preventing Notch upregulation of Tbx21,” Nature Immunology 2005, 7:680-688. In vitro, gamma-secretase inhibitors extinguished expression of Notch, interferon-gamma and Tbx21 in TH1-polarized CD4+ cells. In vivo, administration of gamma-secretase inhibitors substantially impeded TH1-mediated disease progression in the mouse experimental autoimmune encephalomyelitis model of multiple sclerosis suggesting the possibility of using such compounds to treat TH1-mediated autoimmunity See Id. Inhibition of gamma-secretase can alter lymphopoiesis and intestinal cell differentiation (Wong et al., “Chronic Treatment with the γ-Secretase Inhibitor LY-411,575 Inhibits β-Amyloid Peptide Production and Alters Lymphopoiesis and Intestinal Cell Differentiation” Journal of Biological Chemistry 2004, 26:12876-12882), including the induction of goblet cell metaplasia. See Milano et al., “Modulation of Notch Processing by g-Secretase Inhibitors Causes Intestinal Goblet Cell Metaplasia and Induction of Genes Known to Specify Gut Secretory Lineage Differentiation” Toxicological Sciences 2004, 82:341-358.

Strategies that can alter amyloid precursor protein (“APP”) processing and reduce the production of pathogenic forms of amyloid-beta without significantly affecting Notch processing are highly desirable. Moreover, as described above, the inhibition of gamma-secretase has been shown in vitro and in vivo to inhibit the polarization of Th cells and is therefore useful for treating disorders associated with Th1 cells. Th1 cells are involved in the pathogenesis of a variety of organ-specific autoimmune disorders, Crohn's disease, Helicobacter pylori-induced peptic ulcer, acute kidney allograft rejection, and unexplained recurrent abortions, to name a few.

According to one embodiment, the invention relates to a method of inhibiting the formation of Th1 cells in a patient comprising the step of administering to said patient a compound of the present invention, or a composition comprising said compound. In certain embodiments, the present invention provides a method for treating one or more autoimmune disorders, including irritable bowel disorder, Crohn's disease, rheumatoid arthritis, psoriasis, Helicobacter pylori-induced peptic ulcer, acute kidney allograft rejection, multiple sclerosis, or systemic lupus erythematosus, wherein said method comprises administering to said patient a provided compound, prepared according to methods of the present invention, or a pharmaceutically acceptable composition comprising said compound.

In certain embodiments, the present invention provides a method for modulating and/or inhibiting amyloid-beta peptide production, without affecting Notch processing, in a patient, wherein said method comprises administering to said patient a provided compound, or a pharmaceutically acceptable composition comprising said compound. In certain embodiments, the present invention provides a method for modulating and/or inhibiting amyloid-beta peptide production, wherein said method does not substantially reduce processing of Notch and other substrates of gamma secretase.

In certain embodiments, the present invention provides a method for inhibiting amyloid-beta (1-42) peptide production, without affecting the release of Notch intracellular domain (NICD) following the processing of Notch, in a patient, wherein said method comprises administering to said patient a provided compound, or a pharmaceutically acceptable composition comprising said compound.

In certain embodiments, the present invention provides a method for reducing amyloid-beta (1-42) peptide levels in a patient and increasing one or more of amyloid-beta (1-37) and amyloid-beta (1-39), without affecting the release of Notch intracellular domain (NICD) following the processing of Notch, wherein said method comprises administering to said patient a provided compound, or a pharmaceutically acceptable composition thereof.

Accordingly, another aspect of the present invention provides a method for reducing the ratio of amyloid-beta (1-42) peptide to amyloid-beta (1-40) peptide in a patient, without affecting the release of Notch intracellular domain (NICD) following the processing of Notch, comprising administering to said patient a provided compound, or a pharmaceutically acceptable composition thereof. In certain embodiments, the ratio of amyloid-beta (1-42) peptide to amyloid-beta (1-40) peptide is reduced from a range of about 0.1 to about 0.4 to a range of about 0.05 to about 0.08

In some embodiments, the present invention provides a method of treating a disease or disorder in a subject, comprising administering to the subject a compound of formula I, wherein the disease or disorder is Alzheimer's Disease (AD), cerebral amyloid angiopathy, Hereditary Cerebral Hemorrhage with Amyloidosis of the Dutch-Type (HCHWA-D), multi-infarct dementia, dementia pugilistica, traumatic brain injury, Down syndrome, Parkinson's disease, mild cognitive impairment, age-related cognitive decline, pre-symptomatic AD, prodromal or predementia AD, Lewy body dementia, cataract (where a-beta is aggregating in the eye lens), age-related macular degeneration, Tauopathies, Huntington's disease, ALS/Lou Gerhig's disease, Type 2 diabetes (IAPP aggregates in pancreatic islets, is similar in size and sequence to A-beta and having type 2 diabetes increases risk of dementia), transthyretin amyloid disease (TTR, an example of this disease is in heart muscle contributing to cardiomyopathy), prion disease, Gerstmann-Sträussler-Scheinker syndrome, fatal familial insomnia, kuru, CJD, degenerative dementia, Creutzfeld-Jakob disease, amyotrophic lateral sclerosis, progressive supranuclear palsy, head trauma, stroke, pancreatitis, inclusion body myositis, other peripheral amyloidoses, diabetes or atherosclerosis.

In certain embodiments, the disease is an autoimmune disease or disorder. For instance, in certain embodiments, the disease or disorder is irritable bowel disorder, Crohn's disease, rheumatoid arthritis, psoriasis, Helicobacter pylori-induced peptic ulcer, acute kidney allograft rejection, multiple sclerosis, or systemic lupus.

The compounds of the invention are preferably formulated in dosage unit form for ease of administration and uniformity of dosage. The expression “dosage unit form” as used herein refers to a physically discrete unit of agent appropriate for the patient to be treated. It will be understood, however, that the total daily usage of the compounds and compositions of the present invention will be decided by the attending physician within the scope of sound medical judgment. The specific effective dose level for any particular patient or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed, and like factors well known in the medical arts. The term “patient,” as used herein, means an animal, preferably a mammal, and most preferably a human.

Various functions and advantages of these and other embodiments of the present invention will be more fully understood from the examples described below. The following examples are intended to illustrate the benefits of the present invention, but do not exemplify the full scope of the invention.

### EXEMPLIFICATION

The black cohosh extract, utilized in the separation protocol described below, was obtained as a custom order from Boehringer Ingelheim Nutriceuticals. This extract is substantially equivalent to the USP preparation of black cohosh extract, in which about 50% aqueous ethanol is used to extract powdered root and rhizome and then concentrated to near dryness.

The following experimentals describe the isolation of compounds for use in methods of the present invention. Melting points are uncorrected. ¹H and ¹³C NMR spectra were measured at 400 and 100 MHz respectively in CDCl₃, CD₃OD, or pyridine-d5. Chemical shifts are downfield from trimethylsilane (TMS) as internal standards, and J values are in hertz. Mass spectra were obtained on API-2000, or Hewlett Parkard series 1100 MSD with ESI technique. All solvents used were reagent grade. Gamma-oryzanol was purchased from ChemPacific Corporation (Baltimore, Md., USA). The black cohosh extract was obtained as a custom order from Hauser Pharmaceuticals. This extract is substantially equivalent to the USP preparation of black cohosh extract, in which about 50% aqueous ethanol is used to extract powdered root and then concentrated to near dryness. Other abbreviations include: Ac₂O (acetic anhydride), DMAP (dimethylaminopyridine), PhI(OAc)₂ (iodosobenzene diacetate), PDC (pyridinium dichromate), TFAA (trifluoroacetic acid), DMDO (dimethyldioxirane), DIPEA (N,N-Diisopropylethylamine), RB (round-bottom), TLC (thin layer chromatography), MeOH (methanol), CD₃OD (methanol d-4), /-PrOH (isopropanol), TBDMS (tert-butyldimethylsilyl-), TBS (tert-butyldimethylsilyl-), DHEA (dehydroepiandrosterone), TBHP (tert-butylhydroperoxide), DMSO (dimethylsulfoxide), KOt-Bu (potassium tert-butoxide), MS (mass spectrometry), Mom-Cl (Chloromethyl methyl ether), EtOAc (ethyl acetate), M.P. (melting point), EtPPh₃I (ethyltriphenylphosphonium iodide), Et₃N (triethyl amine), mCPBA (met[alpha]-chloroperbenzoic acid), BF₃OEt₂ (trifluoroborane etherate), EtOH (ethanol), HPLC (high performance liquid chromatography), LCMS (liquid chromatography mass spectrometry), NMR (nuclear magnetic resonance).

As used herein, the compound numbers recited below correspond to the following compounds:

Compound 1: 24-O-Acetylhydroshengmanol 3-[beta]-D-xylopyranoside. C₃₇H₆₀O₁₁, Mol. Wt. 680.87; Registry 78213-32-8.

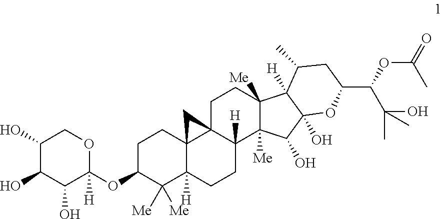

Compound 2: 24-O-Acetylhydroshengmanol 3-[alpha]-L-arabinopyranoside. C₃₇H₆₀O₁₁, Mol. Wt.: 680.87; Registry 915277-93-9.

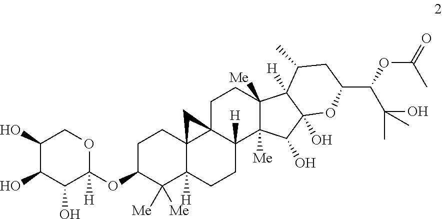

Compound 3: 24-O-Acetylhydroshengmanol 3-[beta]-D-xylopyranoside (delta-16,17)-enol ether. C₃₇H₅₈O₁₀, Mol. Wt.: 662.85; Registry 915277-86-0.

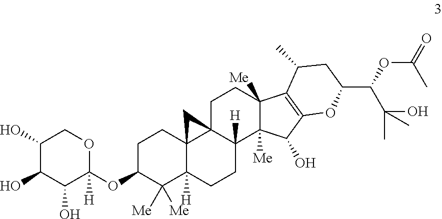

Compound 4: 24-O-Acetylhydroshengmanol 3-[alpha]-L-arabinopyranoside (delta-16,17)-enol ether. C₃₇H₅₈O₁₀, Mol. Wt.: 662.85; 915277-87-1.

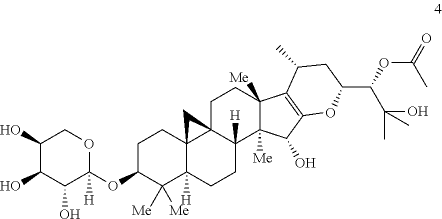

Compound 5: 9,19-Cyclolanostan-15-one, 24-(acetyloxy)-16,23-epoxy-25-hydroxy-3-β3-D-xylopyranosyloxy)-, (3β,16α,17R,23R,24S)-. C₃₇H₅₈O₁₀, Mol. Wt.: 662.85.

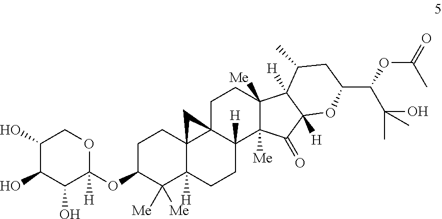

Compound 6: 9,19-Cyclolanostan-15-one, 24-(acetyloxy)-16,23-epoxy-25-hydroxy-3-(α-L-arabinopyranosyloxy)-, (3β,16α,17R,23R,24S)-. C₃₇H₅₈O₁₀, Mol. Wt.: 662.85.

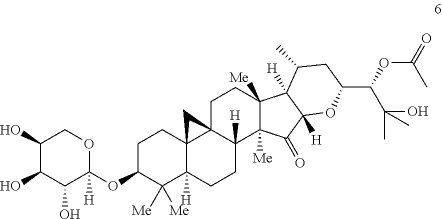

Compound 7: 9,19-Cyclolanostan-15-ol, 24-(acetyloxy)-16,23-epoxy-15,25-hydroxy-3-(β-D-xylopyranosyloxy)-, (3β,15α,16α,17R,23R,24S)-. C₃₇H₆₀O₁₀, Mol. Wt.: 664.87.

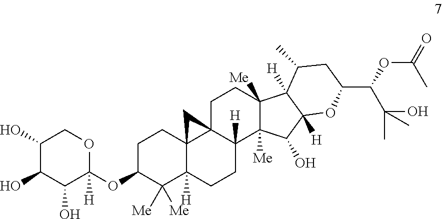

Compound 8: 9,19-Cyclolanostan-15-ol, 24-(acetyloxy)-16,23-epoxy-15,25-hydroxy-3-(α-L-arabinopyranosyloxy)-, (3β,15α,16α,17R,23R,24S)-. C₃₇H₆₀O₁₀, Mol. Wt.:

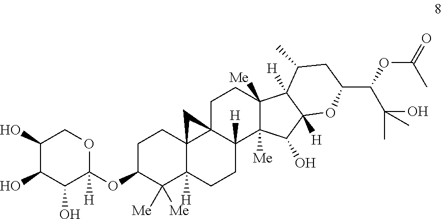

Compound 9: β-D-Xylopyranoside, [Also known as Cimigenoside, 25-acetate; 25-O-Acetylcimigenol 3-O-β-D-xyloside, and 25-O-Acetylcimigenol-3-O-β-D-xylopyranoside. C₃₇H₅₈O₁₀, Mol. Wt.: 662.85; Registry 27994-12-3].

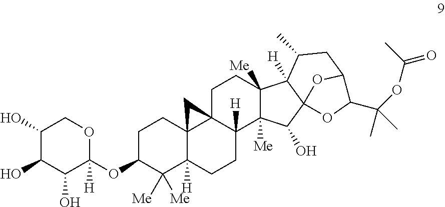

Compound 10: 9,19-Cyclolanostan-15-ol, 24-(acetyloxy)-16,23-epoxy-15,25-hydroxy-3-[2-hydroxy-1S-(2-hydroxyethoxy)ethoxy]-, (3β,15α,16α,17R,23R,24S)-. C₃₆H₆₀O₉, Mol. Wt.: 636.87.

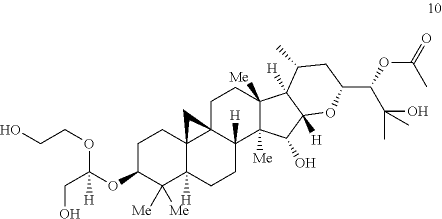

### Example 1

Compound 12 was prepared according to Scheme 8 below.

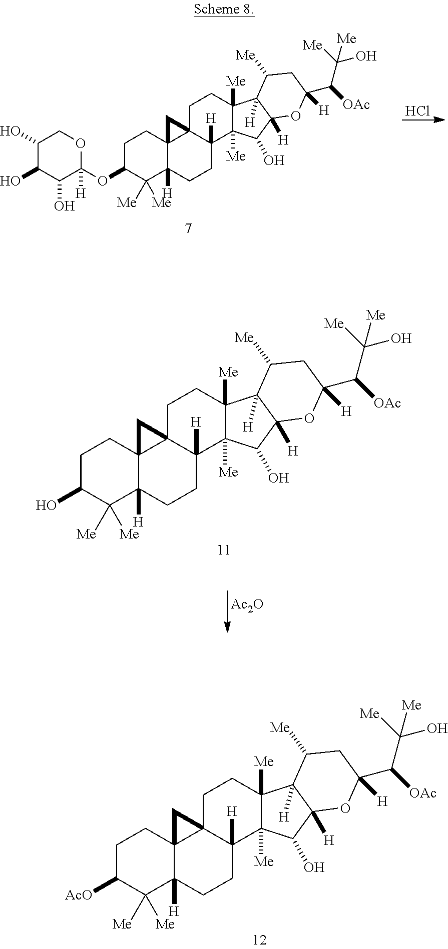

11: Concentrated HCl (0.5 mL) was added to a suspension of 7 (25 mg, 0.038 mmol) in 2 mL of CH₃CN. The mixture was sonicated for 2 minutes to help dissolve 7 then the solution was allowed to stir for 1 h. The solution was then diluted with 50 mL CH₂Cl₂, washed with 50 mL of saturated NaHCO₃, and dried over Na₂SO₄. The crude product was purified by Biotage MPLC eluting with 50-100% ethyl acetate/hexanes to give 14 mg (67%) compound 11. MS (m/z) 555.4 (M+Na)⁺.

12: Acetic anhydride (3.7 μL, 0.039 mmol) was added to a solution of 11 (20 mg, 0.038 mmol) and DMAP (4.8 mg, 0.039 mmol) in anhydrous CH₂Cl₂ (0.4 mL). The solution was allowed to stir for 1 h then purified by Biotage MPLC eluting with 0-100% ethyl acetate/hexanes to give 5.5 mg (25%) compound 12. MS (m/z) 597.4 (M+Na)⁺.

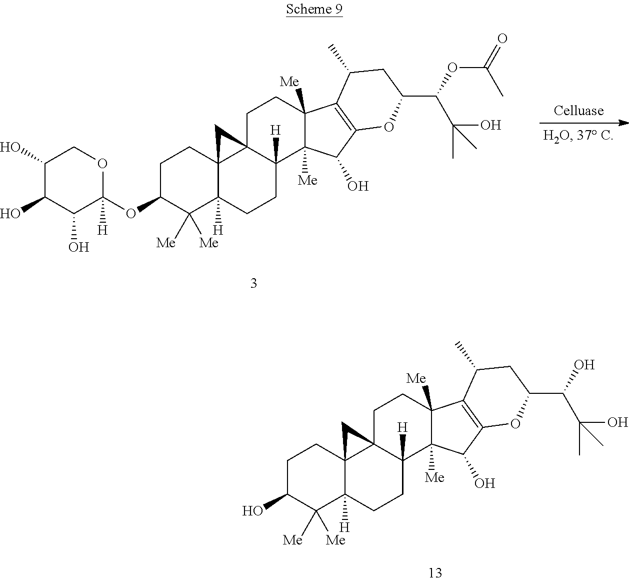

Compound 3 (100 mg) was dissolved in MeOH (50 mL) and added to aqueous K₃PO₄ (pH 6.0, 100 mL). Cellulase (200 mg) dissolved in aqueous KH₂PO₄ (pH 6.0, 100 mL) was then added to the solution containing compound 3 and the combined mixture was allowed to stir at 37° C. for 3 days. Upon completion of the reaction as determined by HPLC analysis, the solvent was reduced in vacuo and the resulting residue was subjected to silica gel chromatography (0-5% MeOH/CH₂Cl₂) to give compound 13 (54 mg, 70%). m/z=511 (M⁺+Na).

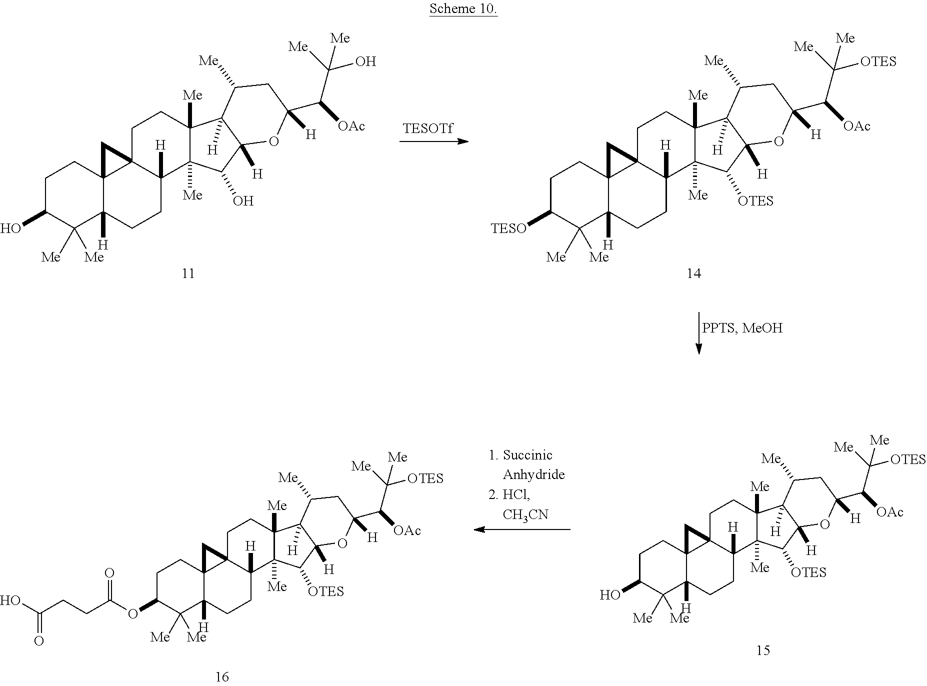

TES-protected compound 16 was prepared according to Scheme 10 above. TESOTf (0.165 mL) was added to a solution of 11 (50 mg, 0.094 mmol) and 2,6-lutidine (0.110 mL) in 1 mL of CH₂Cl₂ at 0° C. After 1 hour the solution was warmed to room temperature and stirred for an additional 1 h then purified by Biotage MPLC eluting with 0-10% ethyl acetate/hexanes to give 113 mg (contains TESOH) 14.

Compound 14 was dissolved in 1 mL of MeOH and 1 mL of CH₂Cl₂, then PPTS (10 mg) was added and the solution was allowed to stir for 5 min. The solution was diluted with 50 mL CH₂Cl₂ and washed with 50 mL of saturated NaHCO₃, and dried over Na₂SO₄. The crude product was purified by Biotage MPLC eluting with 0-50% ethyl acetate/hexanes to give compound 15 (7.0 mg).

16: Succinic anhydride (25 mg, 0.20 mmol) was added to a solution of compound 15 (7.0 mg, 0.0091 mmol) and DMAP (30 mg, 0.20 mmol) in CH₂Cl₂ (0.5 mL). The solution was allowed to stir for 1 h then the solution was then diluted with 50 mL CH₂Cl₂, washed with 50 mL of 1 N HCl, and dried over Na₂SO₄. The crude product in 4 mL of CH₃CN was treated with 1 mL of concentrated HCl, and the solution was allowed to stir for 10 minutes. The solution was then diluted with 50 mL CH₂Cl₂, washed with 50 mL of water, and dried over Na₂SO₄. The crude product was purified by Biotage MPLC eluting with 0-100% ethyl acetate (1% added formic acid)/hexanes to give compound 16. MS (m/z) 655.4 (M+Na)⁺.

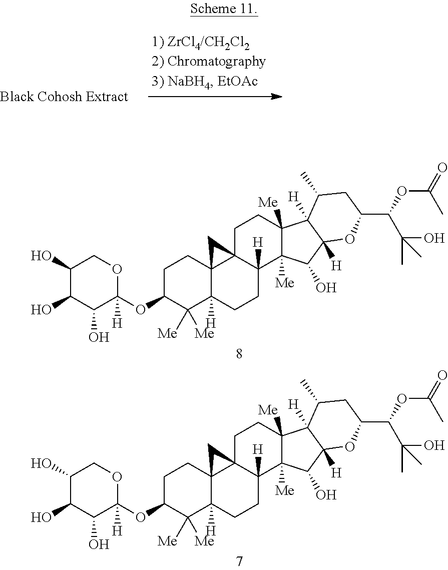

Black cohosh extract (49 g) was ground to a fine powder with a mortar and pestle and suspended in 10% MeOH/CH₂Cl₂ (200 mL). The suspension was stirred at room temperature for 2 h and then vacuum filtered through a pad of celite. The resulting clear solution was evaporated in vacuo to give an orange/brown solid. The material was dissolved in CH₂Cl₂ (800 mL) and ZrCl₄ (660 mg) was added. The solution was stirred at room temperature for 2 h whereupon the solvent was reduced in vacuo. The orange/brown solid was then subjected to column chromatography on silica gel using 5-8% MeOH/CH₂Cl₂. All fractions corresponding to reference standards of compounds 5 and 6 by TLC analysis (2×7% MeOH/CH₂Cl₂) were combined and the solvent was reduced in vacuo. The resulting solid was dried under high vacuum and residue was then dissolved in EtOAc (7 mL) and NaBH₄ (50 mg) was added. The suspension was stirred at room temperature overnight and the solvent was then removed in vacuo. The solid was dissolved in CH₂Cl₂ (7 mL) and cooled to 4° C. The chilled solution was then added drop wise to an ice chilled aqueous solution of 10% citric acid (3 mL) in a separating funnel which caused vigorous bubbling. Once all of the solution had been added and bubbling had ceased, the organic layer was separated. The solvent was then removed in vacuo and the residue was purified by silica gel chromatography (5-10% MeOH in CH₂Cl₂) to give compounds 8 and 7 as a combined sample (2.11 g). m/z=687 (M⁺+Na).

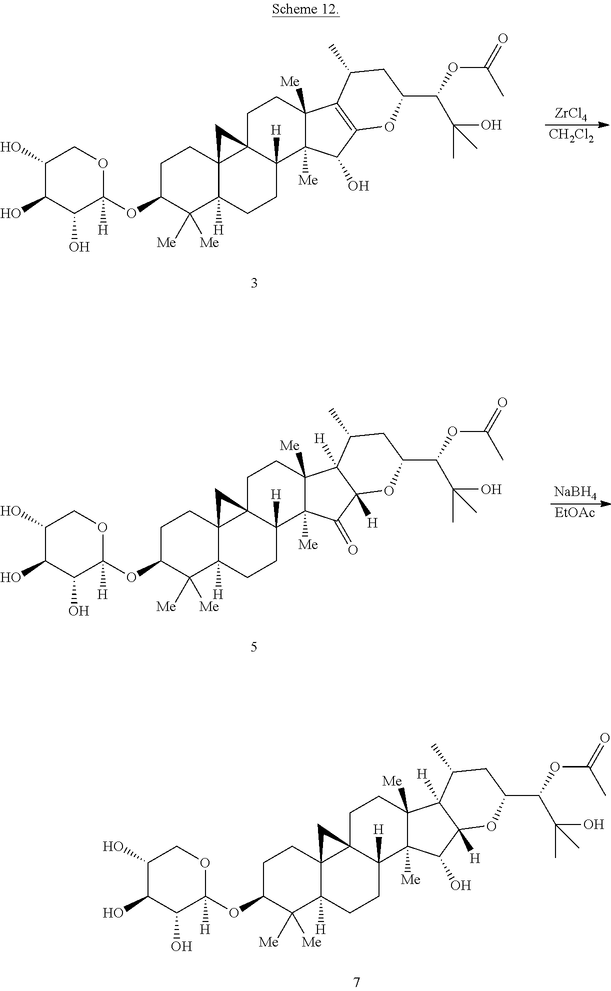

To compound 3 (0.03 g) and ZrCl₄ (1.4 mg) was added CH₂Cl₂ (4 mL). The solution was ultra-sonicated for 2 minutes and then stirred vigorously for 1 hour. The solvent was then removed in vacuo and redissolved in EtOAc (6 mL). NaBH₄ (0.05 g) was then added and the solution was ultra-sonicated for at least 2 minutes and the reaction mixture was allowed to stir overnight at room temperature. The solvent was removed in vacuo and the residue was redissolved in CH₂Cl₂ (4 mL). The solution was then added drop wise to an ice chilled aqueous solution of 5% citric acid (2 mL) in a separating funnel which caused vigorous bubbling. Once all of the solution had been added and bubbling had ceased, the organic layer was separated. The solvent was then removed in vacuo and the residue was purified by silica gel chromatography (5-10% MeOH in CH₂Cl₂) to give compound 7. m/z=687 (M⁺+Na).

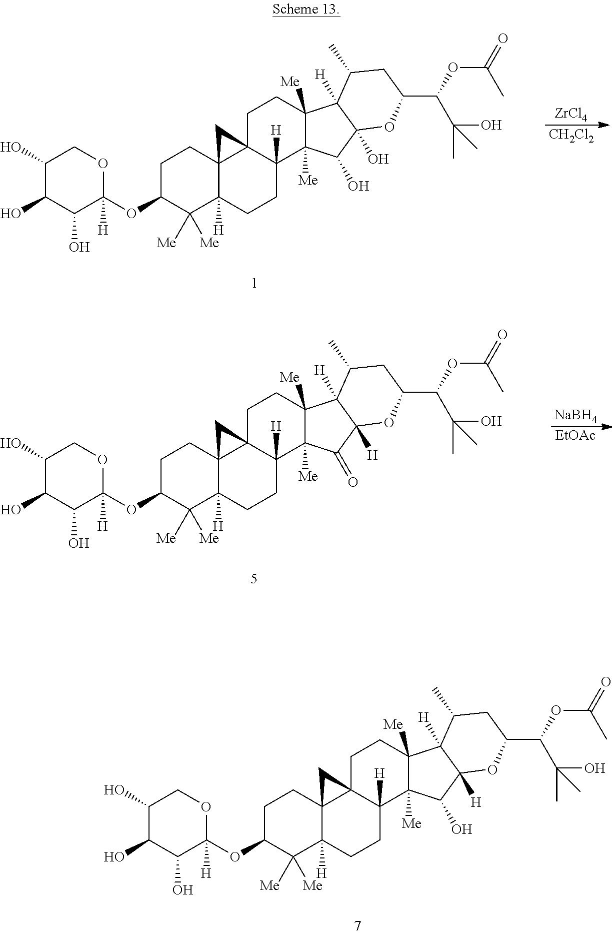

Compound 7 may also be synthesized from compound 1 under the same procedure outlined in Scheme 12. Similarly, compound 8 may be synthesized from compounds 2 or 4 using the procedure from Scheme 12.

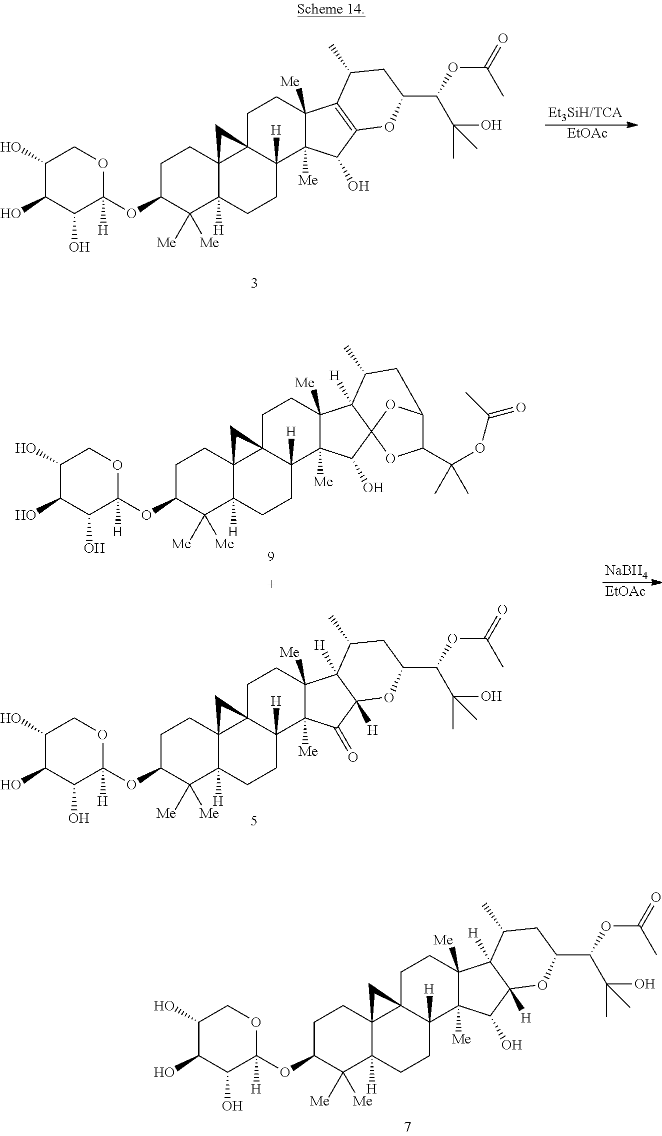

To compound 3 (0.1 g) in EtOAc (50 mL) was added triethylsilane (100 μL) followed by dry trichloroacetic acid (TCA) (55 mg). The cloudy solution was stirred at room temperature over night under nitrogen. TLC analysis indicated a 1:1 mixture of compounds 9 and 5. The solvent was removed in vacuo and the residue was purified by silica gel chromatography (5-10% MeOH in CH₂Cl₂) to give compounds 5 and 9 as a single sample. The collected residue was redissolved in EtOAc (60 mL). NaBH₄ (0.5 g) was then added and the solution was then ultra-sonicated for at least 2 minutes and the reaction mixture was allowed to stir overnight at room temperature. The solvent was removed in vacuo and the residue was redissolved in CH₂Cl₂ (40 mL). The solution was then added drop wise to an ice chilled aqueous solution of 5% citric acid (20 mL) in a separating funnel which caused vigorous bubbling. Once all of the solution had been added and bubbling had ceased, the organic layer was separated. The solvent was then removed in vacuo and the residue was purified by silica gel chromatography (5-10% MeOH in CH₂Cl₂) to give compound 7 (0.04 g, 40%). m/z=687 (M⁺+Na).

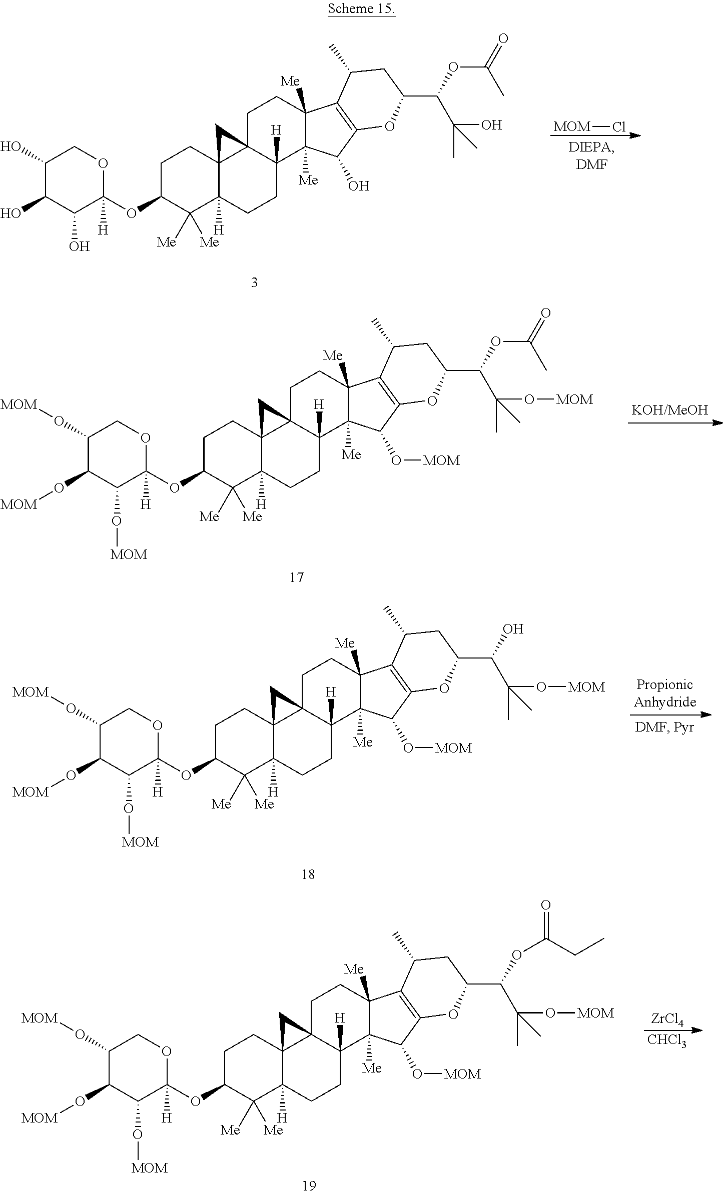

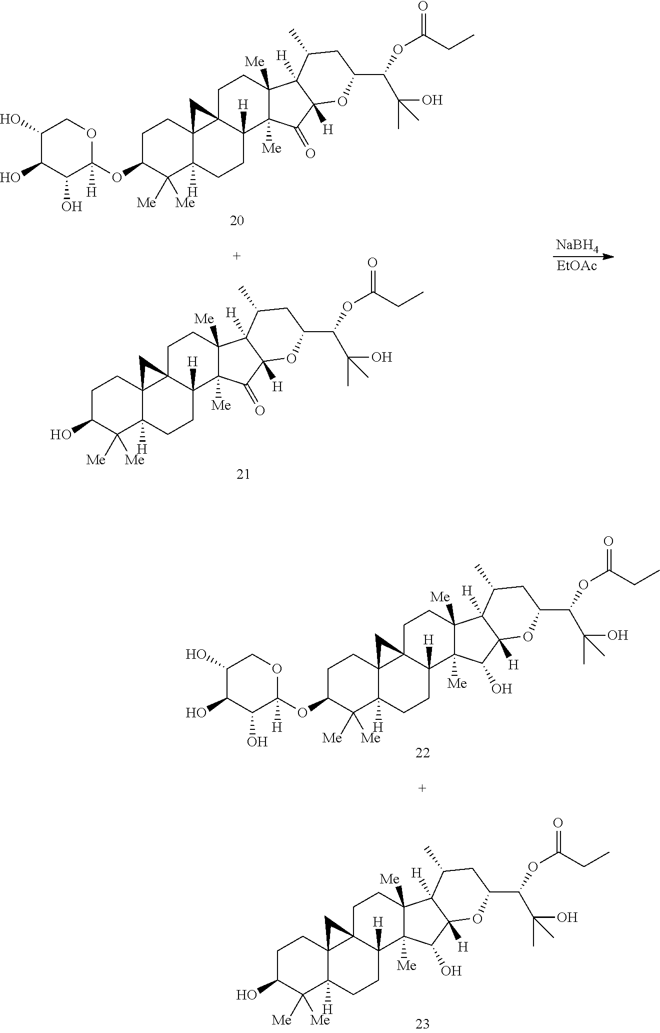

Compound 3 (151 mg) and DMAP (4.8 mg) was dissolved in DMF (3 mL) with stirring under argon. DIPEA (750 μl) was then added and the solution was stirred for 10 minutes. Mom-Cl (210 μL) was added to the reaction mixture and the solution was allowed to stir at room temperature for 4 days. Additional MOM-Cl (105 μL) was added and the reaction mixture was stirred for a further 2 days. The solvent was removed in vacuo and the residue was dissolved in CH₂Cl₂ (40 mL) and washed sequentially with H₂O (30 mL) and 10% Na₂CO₃ (30 mL). Removal of the solvent gave a residue that was subjected to silica gel chromatography (5-10% MeOH in CH₂Cl₂) to give compound 17 as a single product [m/z=906 (M⁺+Na)]. Compound 17 was dissolved in methanol (30 mL) and treated with solid KOH at room temperature until the complete removal of the acetate, as indicated by TLC analysis, gave compound 18. The solvent was then removed and the residue dissolved in CH₂Cl₂ and washed twice with H₂O. The organic solvent was then removed and the residue was dried under high vacuum. Approximately 50% of the residue was then dissolved in DMF (2 mL) and treated with pyridine (300 μL), propionic anhydride (300 μL) and DMAP (15 mg) and the reaction was left to stir for 3 days. The solvent was then removed in vacuo and the residue was dissolved in CH₂Cl₂ (20 mL) and washed with 5% citric acid (20 mL). The removal of the solvent in vacuo gave compound 19 (m/z=919, M⁺+Na). This material was dissolved in CHCl₃ (30 mL) and ZrCl₄ (50 mg) was added. The solution was allowed to stir at 50° C. overnight or until the complete removal of the mom-protecting groups as indicated by TLC analysis. Both compounds 20 and 21 were isolated following silica gel chromatography (0-8% MeOH/CH₂Cl₂). Each compound was individually redissolved in EtOAc (15 mL) and NaBH₄ (0.15 g) was added. The solutions were ultra-sonicated for at least 2 minutes and the reaction mixtures were allowed to stir overnight at room temperature. The solvent was removed in vacuo and each residue was redissolved in CH₂Cl₂ (15 mL). Each solution was then added drop wise to an ice chilled aqueous solutions of 10% citric acid (20 mL) in separating funnels which caused vigorous bubbling. The organic layers were each separated and the solvent was then removed in vacuo. Silica gel chromatography of each product separately gave compounds 22 [m/z=701 (M⁺+Na)] and 23 [m/z=569 (M⁺+Na)].

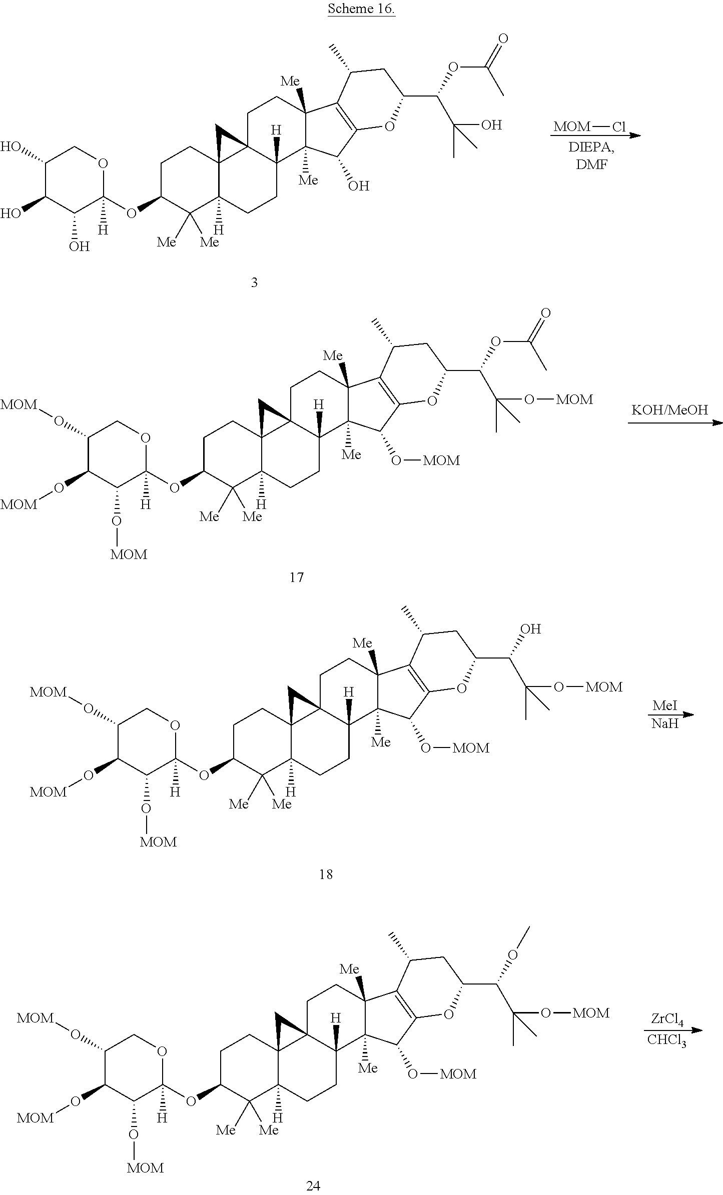

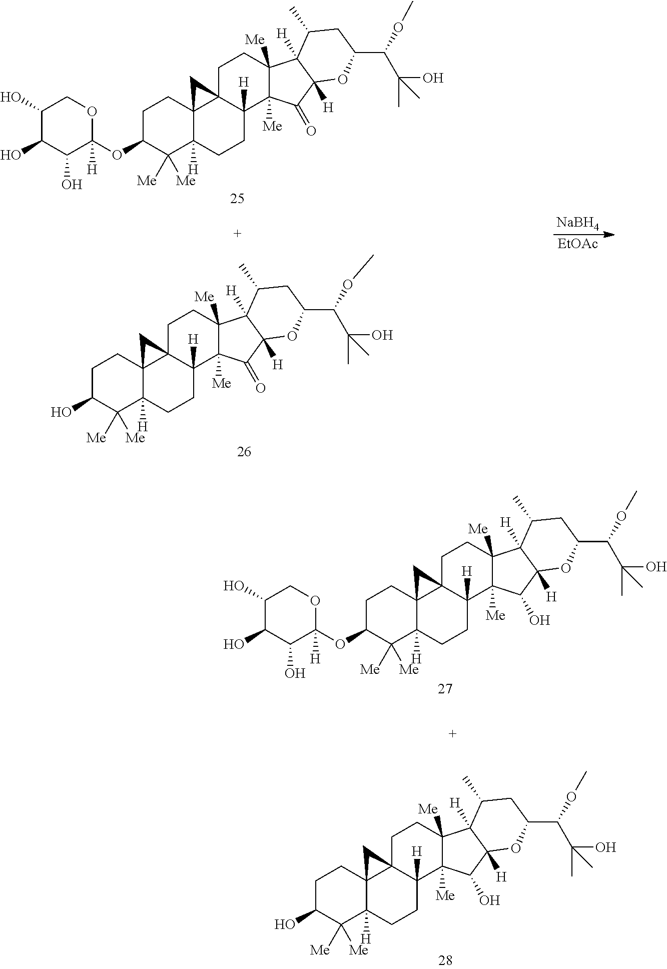

Compound 3 (151 mg) and DMAP (4.8 mg) was dissolved in DMF (3 mL) with stirring under argon. DIPEA (750 μl) was then added and the solution was stirred for 10 minutes. MOM-Cl (210 μL) was added to the reaction mixture and the solution was allowed to stir at room temperature for 4 days. Additional MOM-Cl (105 μL) was added and the reaction mixture was stirred for a further 2 days. The solvent was removed in vacuo and the residue was dissolved in CH₂Cl₂ (40 mL) and washed sequentially with H₂O (30 mL) and 10% Na₂CO₃ (30 mL). Removal of the solvent gave a residue that was subjected to silica gel chromatography (5-10% MeOH in CH₂Cl₂) to give compound 17 as a single product [m/z=906 (M⁺+Na)]. Compound 17 was dissolved in methanol (30 mL) and treated with solid KOH at room temperature until the complete removal of the acetate, as indicated by TLC analysis, gave compound 18. The solvent was then removed and the residue dissolved in CH₂Cl₂ and washed twice with H₂O. The organic solvent was then removed and the residue was dried under high vacuum. Approximately 50% of the residue was dissolved in MeI (3 mL) and treated with NaH. The reaction mixture was allowed to stir for 3 days whereupon the solvent was removed in vacuo and the residue was dissolved in CH₂Cl₂ (20 mL) and washed with 5% citric acid (20 mL). The removal of the solvent in vacuo gave compound 24 (m/z=877, M⁺+Na). This material was dissolved in CHCl₃ (30 mL) and ZrCl₄ (50 mg) was added. The solution was allowed to stir at 50° C. overnight or until the complete removal of the Mom-protecting groups as indicated by TLC analysis. Both compounds 25 and 26 were isolated following silica gel chromatography (0-8% MeOH/CH₂Cl₂). Each compound was individually redissolved in EtOAc (15 mL) and NaBH₄ (0.15 g) was added. The solutions were ultra-sonicated for at least 2 minutes and the reaction mixtures were allowed to stir overnight at room temperature. The solvent was removed in vacuo and each residue was redissolved in CH₂Cl₂ (15 mL). Each solution was then added drop wise to an ice chilled aqueous solutions of 10% citric acid (20 mL) in separating funnels which caused vigorous bubbling. The organic layers were each separated and the solvent was then removed in vacuo. Silica gel chromatography of each product separately gave compounds 27 [m/z=660 (M⁺+Na)] and 28 [m/z=527 (M⁺+Na)].

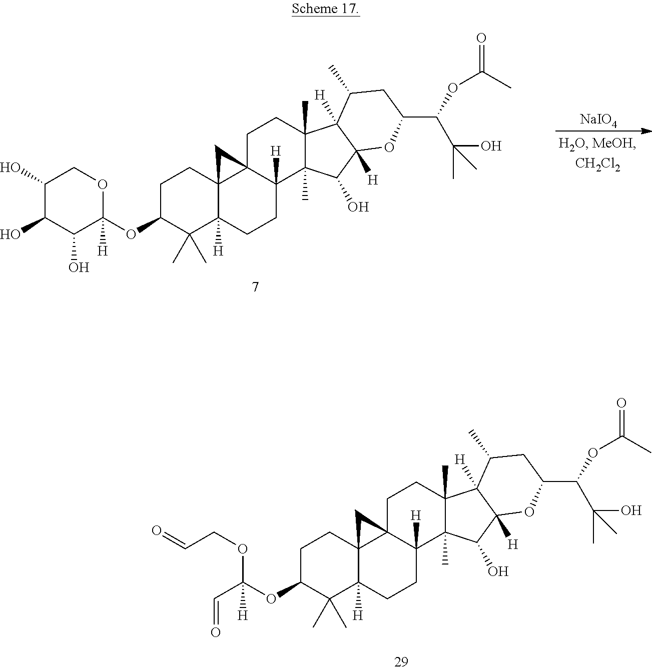

Compound 7 (0.084 g) was dissolved in MeOH (10 mL) and 0.05 mL of an aqueous solution of NaIO₄ (0.02 g in 0.09 mL H₂O) was added drop wise with vigorous stirring and the solution was allowed to stir overnight. An additional 3 mL of aqueous NaIO₄ solution was added, followed by CH₂Cl₂ (0.05 mL) and the solution was stirred for an additional 2 days. The solvent was then removed in vacuo and the resulting residue was dissolved in a minimal amount of 2% methanol/CH₂Cl₂ and purified by silica gel chromatography (2-8% MeOH/CH₂Cl₂) to give compound 29.

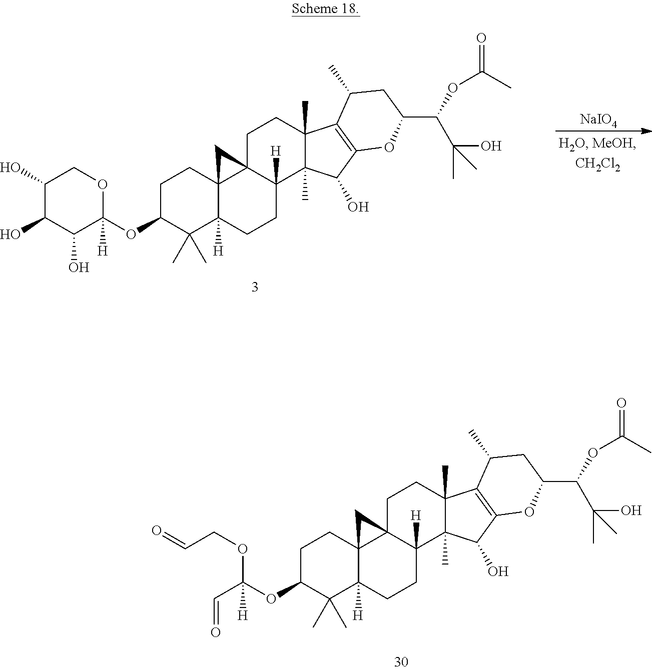

Compound 30 may also be synthesized from compound 3 under the same conditions outlined in Scheme 18.

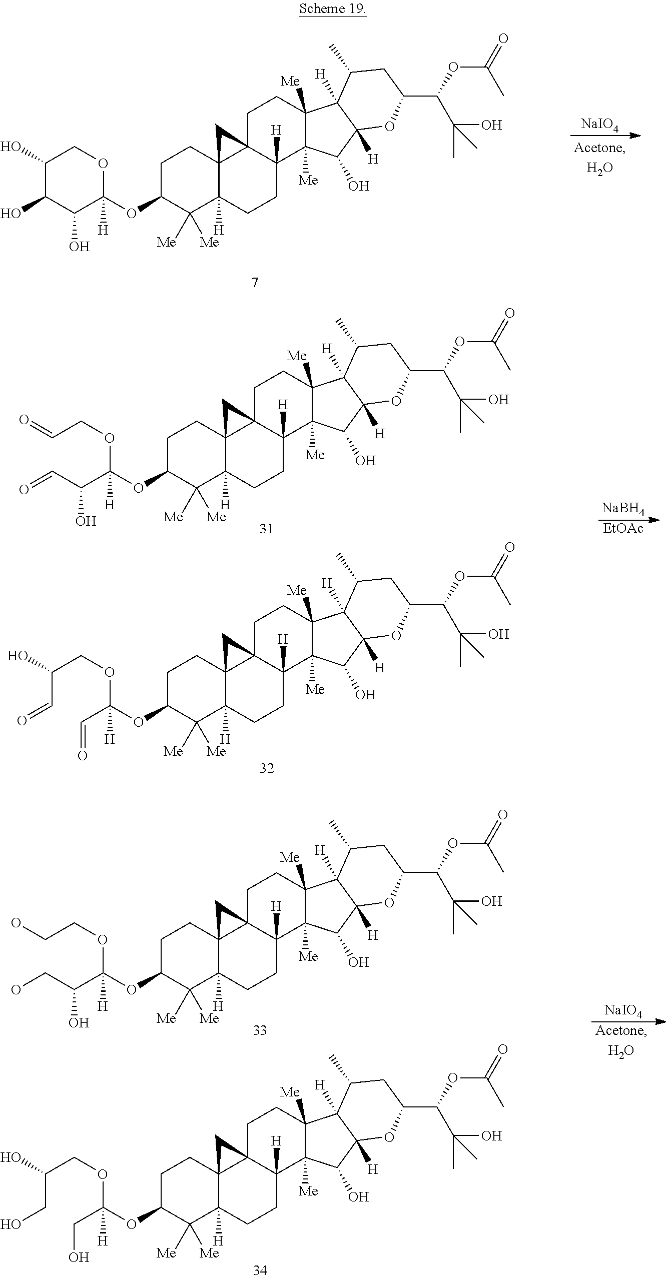

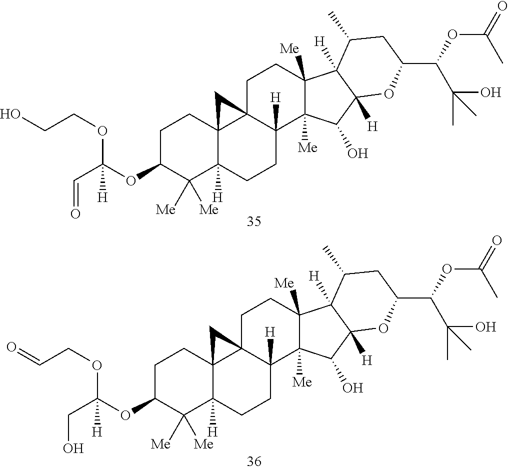

NaIO₄ (0.3 g) was dissolved in H₂O (2 mL) with heating and added drop wise to a stirred solution of compound 7 in acetone (20 mL). The solution was then heated at 60° C. for 4 hours whereupon the solvent was removed in vacuo. The residue was then suspended in 10% MeOH/CH₂Cl₂ and passed through a pad of celite. The solvent was removed in vacuo and the solution was dissolved in EtOAc (20 mL). NaBH₄ (0.33 g) was then added and the solution was ultra-sonicated for 3 minutes and the reaction mixture was allowed to stir overnight at room temperature. The solvent was removed in vacuo and the residue was redissolved in CH₂Cl₂ (20 mL). The solution was then added drop wise to an ice chilled aqueous solution of 10% citric acid (10 mL) in a separating funnel which caused vigorous bubbling. Once all of the solution had been added and bubbling had ceased, the organic layer was separated. The solvent was then removed in vacuo and redissolved in acetone. NaIO₄ (0.3 g) was dissolved in H₂O (2 mL) with heating and added drop wise to the solution. The solution was allowed to stir at room temperature overnight. The solvent was then removed and the residue was subjected to silica gel chromatography (2-8% MeOH/CH₂Cl₂) to give compounds 35 and 36 as a single sample.

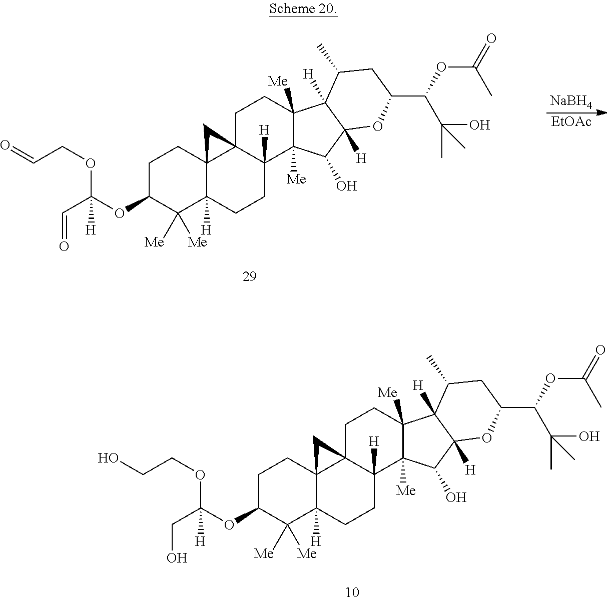

Compound 29 was dissolved in EtOAc (20 mL). NaBH₄ (0.33 g) was then added, the solution was ultra-sonicated for 3 minutes, and the reaction mixture was allowed to stir overnight at room temperature. The solvent was removed in vacuo and the residue was re-dissolved in CH₂Cl₂ (20 mL). The solution was then added drop wise to an ice chilled aqueous solution of 5% citric acid (10 mL) in a separating funnel which caused vigorous bubbling. Once all of the solution had been added and bubbling had ceased, the organic layer was separated. The solvent was then removed in vacuo and the residue was subjected to silica gel chromatography (2-8% MeOH/CH₂Cl₂) to give compound 10. m/z=659 (M⁺+Na).

General Procedure for Reductive Aminations.

A compound containing an aldehyde or di-aldehyde dissolved in MeOH may be treated with an amine (3 mol equivalents), acetic acid (4 mol equivalents) and NaCNBH₃ (3 mol equivalents) as described by Du and Hindsgaul, Synlett, 1997, 395-397 and Anderluh, Tetrahedron Lett., 2006, 47, 9203-9206. The reactions are stirred at room temperature or 80° C. for 3-15 hours and or until complete by LCMS analysis. The solvent is then reduced in vacuo and the resulting amines can be separated by silica gel chromatography or HPLC.

### Scheme 21.

### General Procedure for Reductive Aminations Using Compounds 29 or 30.

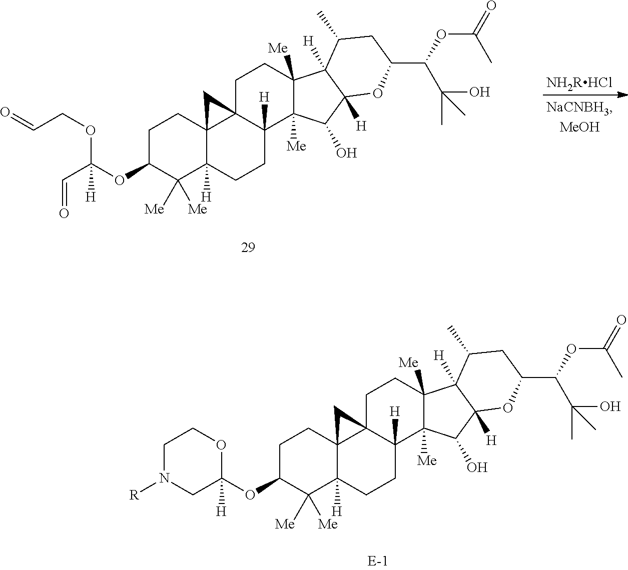

Compound 29, a hydrochloride salt of a primary amine (2 mol equivalents), and NaCNBH₃ (4 mol equivalents) were dissolved in MeOH and stirred at room temperature for 3-8 hours. The solvent was then removed in vacuo and residue was purified by silica gel chromatography (2-5% MeOH/CH₂Cl₂) to give a morpholine-containing product E-1.

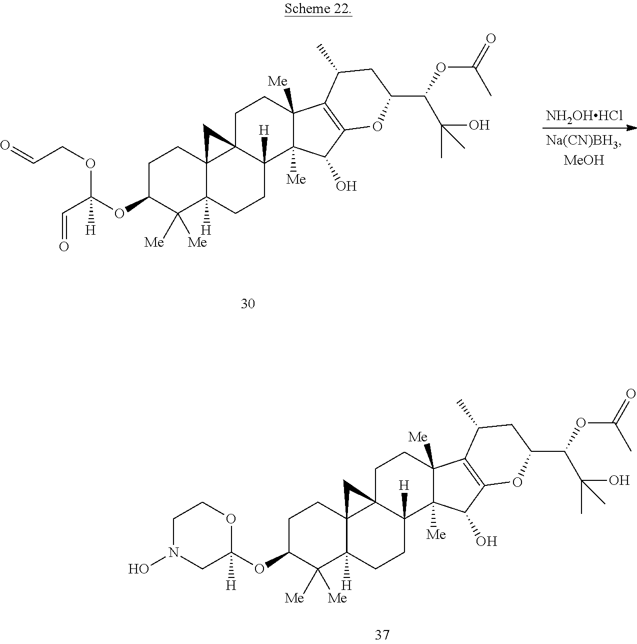

Compound 30 (6.8 mg), hydroxylamine hydrochloride (3.7 mg), and NaCNBH₃ (2.0 mg) were dissolved in MeOH (0.4 mL) and stirred at room temperature for 3 hours. The solvent was then removed in vacuo and the residue was purified by silica gel chromatography (2-5% MeOH/CH₂Cl₂) to give compound 37 (3.0 mg). m/z=654 (M⁺+Na).

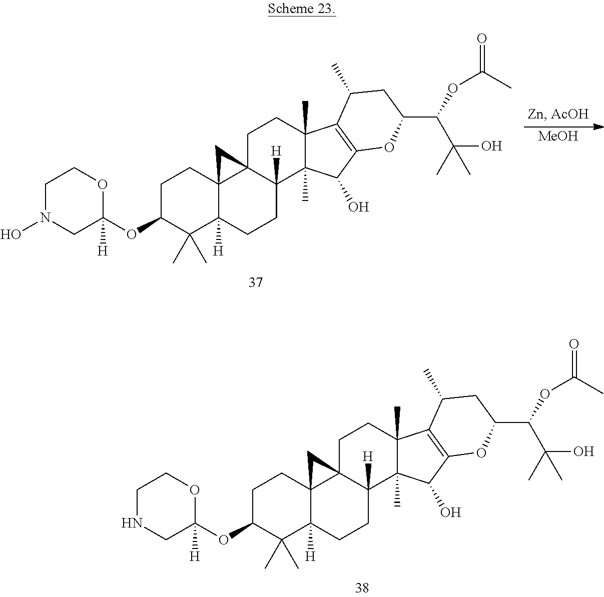

Compound 37 (2.0 mg) was dissolved in MeOH (0.2 mL) and glacial acetic acid (1 μL) and zinc powder (6.5 mg) was added. The solution was then ultra-sonicated for 1 hour at room temperature. The solution was then filtered through a plug of celite and the solvent was removed in vacuo. The resulting residue was purified by silica gel chromatography (0-5% MeOH/CH₂Cl₂) to give compound 38. m/z=618 (M⁺+H).

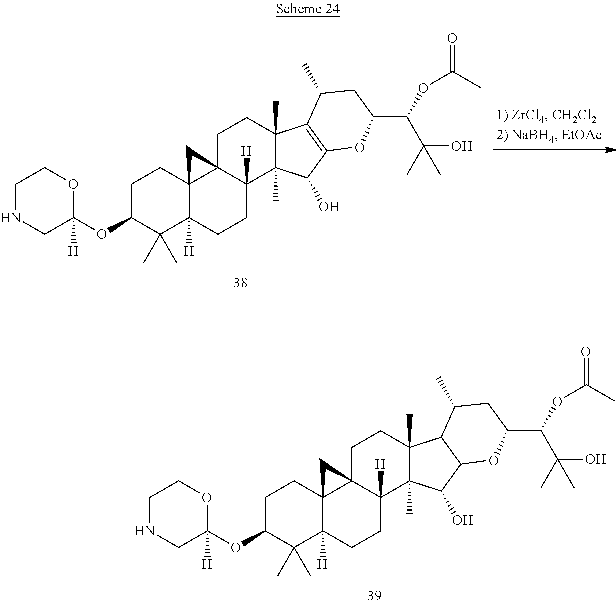

Compound 39 may be synthesized from compound 38 under the same procedure outlined in Scheme 12.

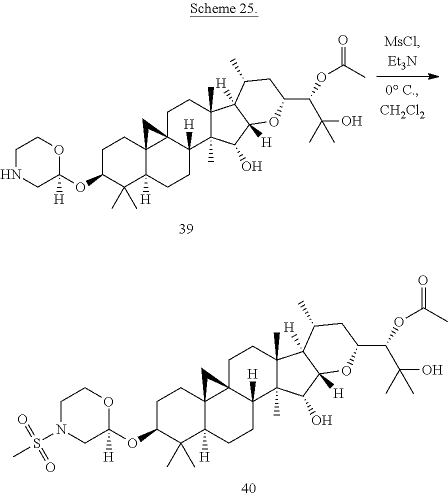

Compound 39 (29 mg) was dissolved in CH₂Cl₂ (5 mL) with triethylamine (24 μL) and stirred at 0° C. under argon. Mesyl chloride (3.6 μL) was then added and the solution temperature was allowed to rise to room temperature over one hour. The solvent was then removed in vacuo and the resulting residue was purified by silica gel chromatography (0-5% MeOH/CH₂Cl₂) to give compound 40. m/z=718 (M⁺+Na).

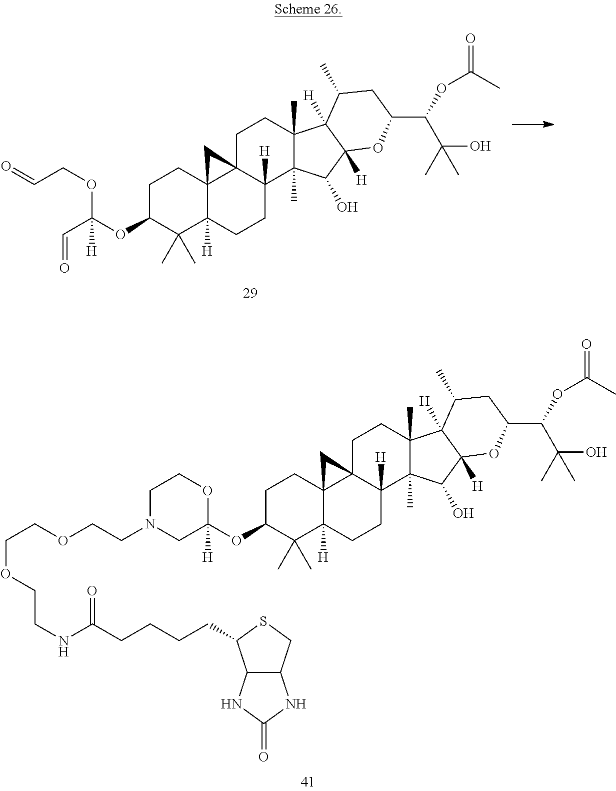

To compound 29 (40 mg) dissolved in MeOH (600 μL) with stirring was added N-biotinyl-3,6-dioxaoctane-1,8-diamine trifluoroacetate salt solution (25 mg/mL in DMSO). NaCNBH₃ (13 mg) was then added and the mixture was stirred at room temperature for 3 hours. The solvents were then removed in vacuo and the resulting residue was purified by silica gel chromatography (0-5% MeOH/CH₂Cl₂) to give compound 41. m/z=997 (M⁺+Na).

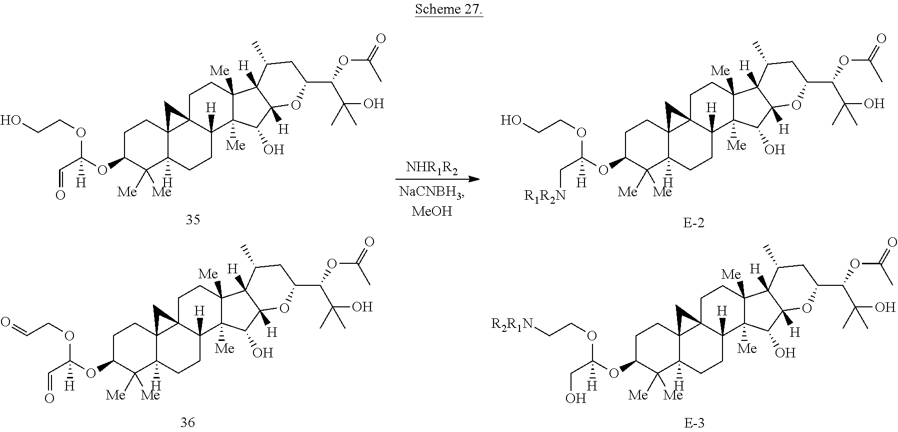

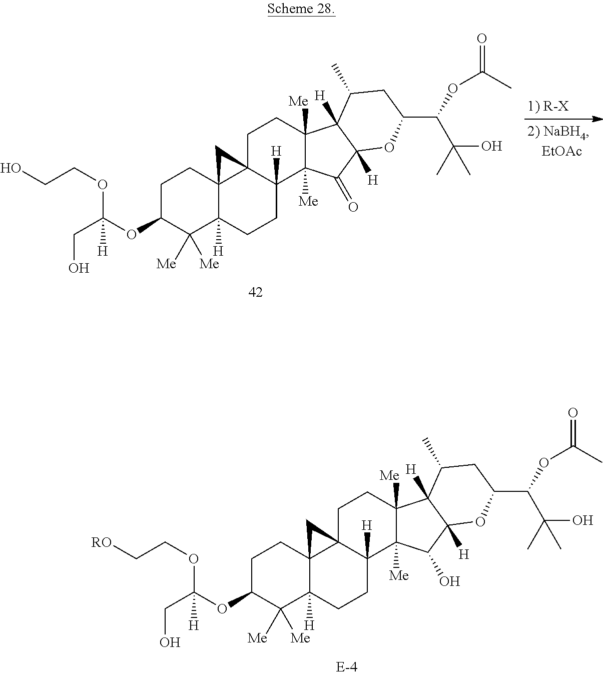

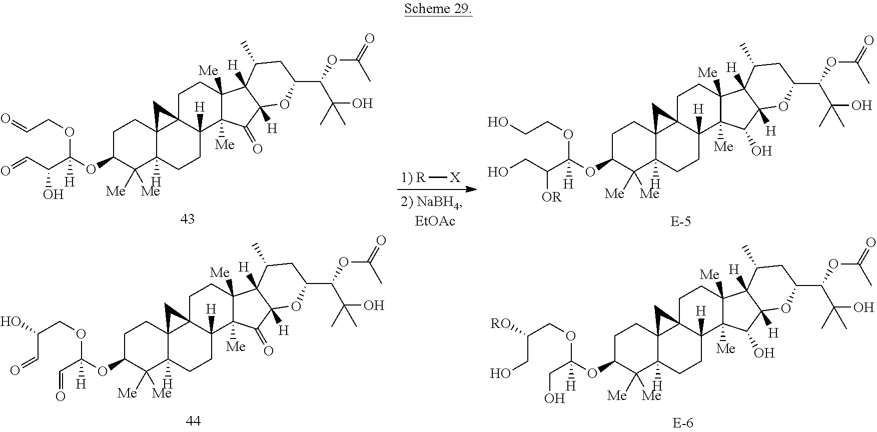

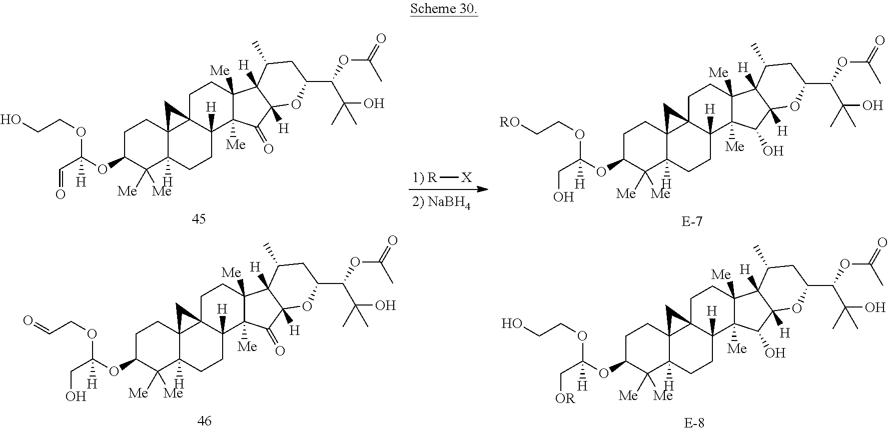

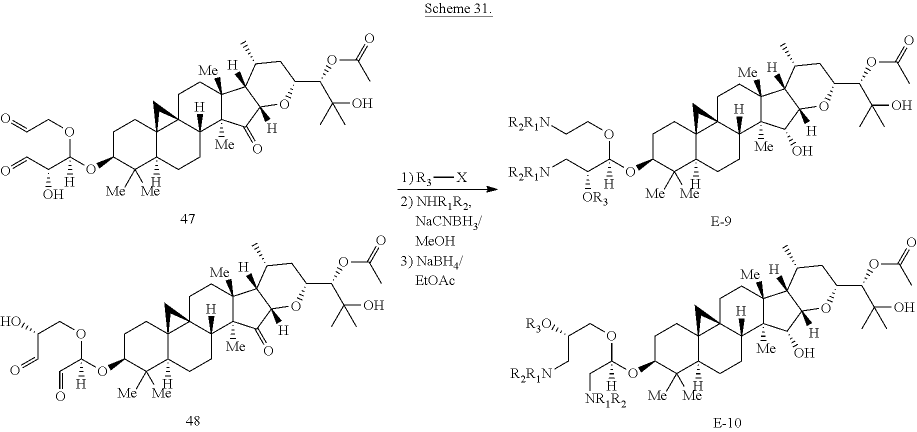

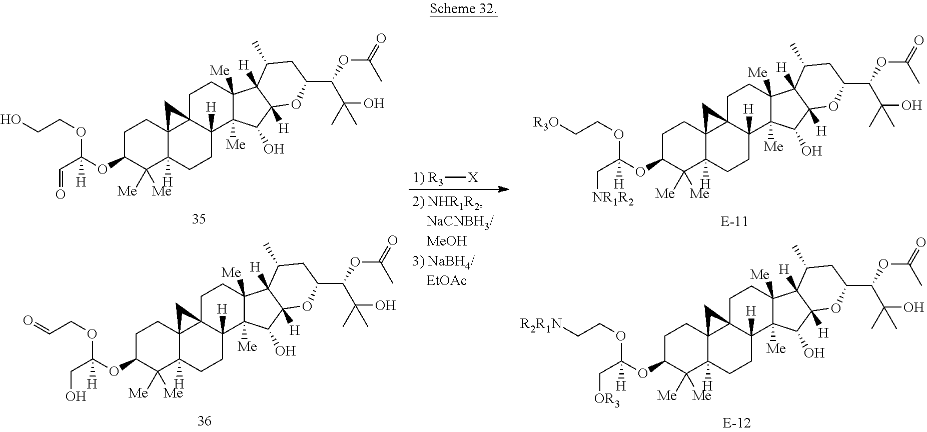

### Example 2

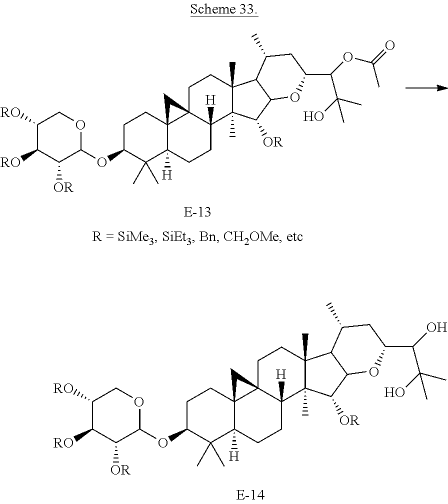

A 25-mL flask is charged with protected polyol E-13 (1 mmol) dissolved in 10 mL of methanol. Potassium carbonate (0.5 g, 3.6 mmol, 3.6 equiv) is added and the resulting mixture is stirred at room temperature until TLC indicates complete consumption of the starting material. The resulting mixture is concentrated under reduced pressure and the residue partitioned between water and organic solvent. The organic phase is concentrated and purified by column chromatography on silica gel to provide the des-acetate E-14.

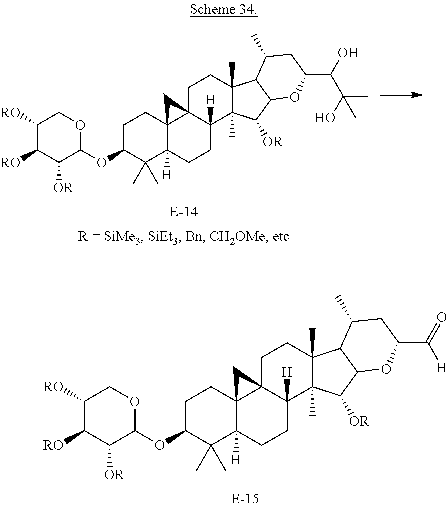

A 250-mL flask is charged with diol E-14 (1 mmol) dissolved in 80 mL of methanol and 20 mL of water. Sodium periodate (2.0 g, 9.3 mmol, 9.3 equiv) is added and the resulting mixture is stirred at room temperature until TLC indicates complete consumption of the starting material. The reaction mixture is concentrated under reduced pressure and the residue partitioned between water and organic solvent. The organic phase is concentrated and purified by column chromatography on silica gel to provide the aldehyde E-15.

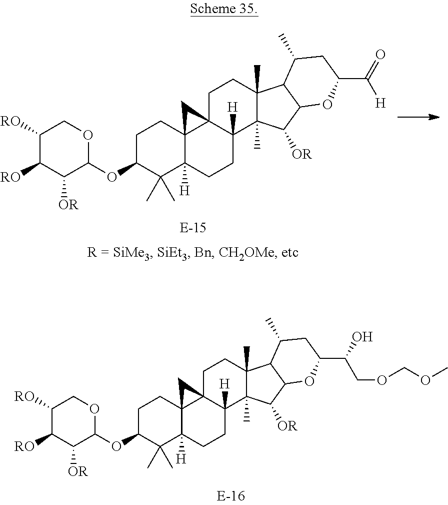

A 10 mL flask is charged with tributyl[(methoxymethoxy)methyl]stannane (0.43 g, 1.2 mmol, 1.2 equiv) in 5 mL of THF and cooled at −78° C. while a solution of n-butyllithium in hexanes (1.1 mmol, 1.1 equiv) is added dropwise. The resulting mixture is stirred at −78° C. for 30 minutes. A separate 50-mL flask is charged with aldehyde E-15 (1 mmol) dissolved in 10 mL of THF and cooled at −78° C. The (methoxymethyoxy)methyl lithium solution is added dropwise, and the reaction mixture is stirred while warming slowly to 0° C. Stirring is continued until TLC indicates complete consumption of the starting material. The resulting mixture is partitioned between water and ether. The organic phase is concentrated and purified by column chromatography on silica gel to provide the alcohol E-16.

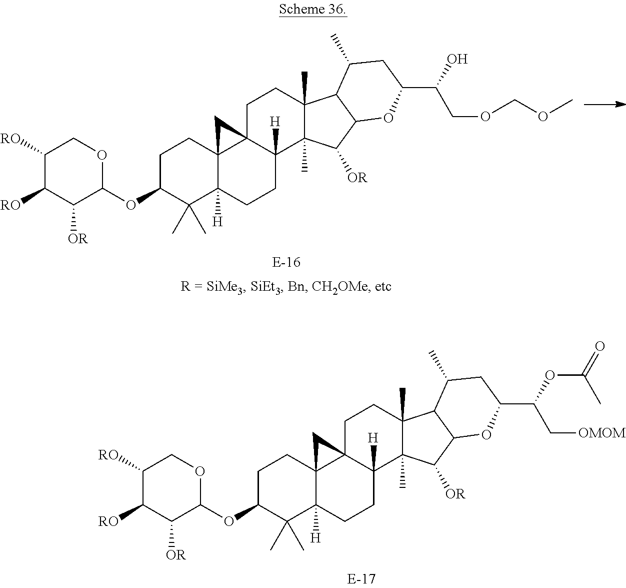

A 25-mL flask is charged with alcohol E-16 (1 mmol) dissolved in 10 mL of dichloromethane and cooled at 0° C. DMAP (0.18 g, 1.5 mmol) is added, followed by 0.14 mL acetic anhydride (150 mg, 1.5 mmol, 1.5 equiv) and the reaction mixture is stirred at room temperature until TLC indicates complete consumption of the starting material. The resulting mixture is partitioned between water and dichloromethane. The organic phase is concentrated and purified by column chromatography on silica gel to provide the acetate E-17.

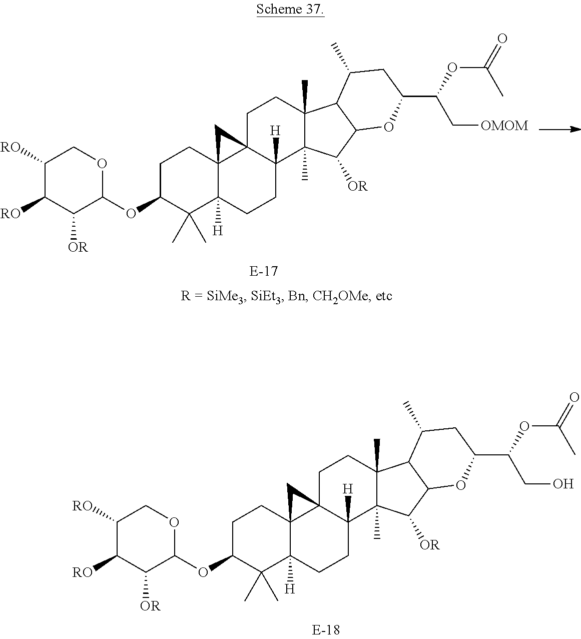

A 25-mL flask is charged with alcohol E-17 (1 mmol) dissolved in 10 mL of THF and stirred at room temperature while 1 mL of 6 M HCl solution is added, and the resulting mixture is stirred at room temperature until TLC indicates complete consumption of the starting material. The reaction mixture is partitioned between water and ether and concentrated. The residue is purified by silica gel chromatography to provide the alcohol E-18.

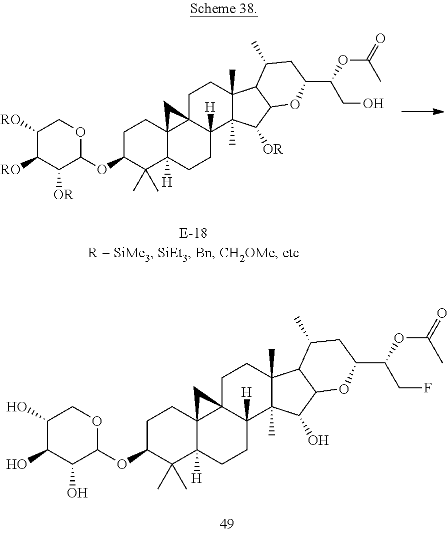

A 25-mL flask is charged with protected polyol E-18 (1 mmol) dissolved in 10 mL of dichloromethane and cooled to −20° C. A nucleophilic fluorinating agent (1.1 mmol) is added and the reaction mixture is allowed to warm to room temperature and stirred until TLC indicates complete consumption of the starting material. The resulting mixture is partitioned between water and dichloromethane and concentrated under reduced pressure. The residue is subjected to the appropriate conditions for removal of the hydroxyl protecting groups and purified by column chromatography to provide the fluoride 49.

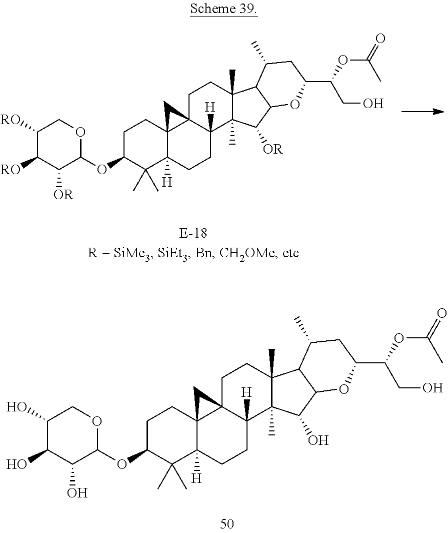

A 25-mL flask is charged with protected polyol E-18 (1 mmol) and subjected to the appropriate conditions for the removal of the hydroxyl protecting groups. The resulting mixture is partitioned between water and organic solvent, the organic phase is concentrated and the residue is purified by column chromatography on silica gel to provide the polyol 50.

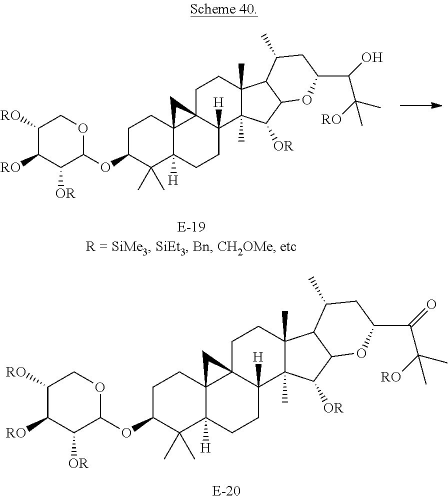

A 25-mL flask is charged with steroid E-19 (1 mmol) dissolved in 10 mL of DMSO and cooled at 0° C. Iodosobenzoic acid (0.40 g, 1.5 mmol) is added and the resulting mixture is stirred at room temperature until TLC indicates complete consumption of the starting material. The reaction mixture is partitioned between water and dichloromethane. The organic phase is concentrated and purified by column chromatography on silica gel to provide the ketone E-20.

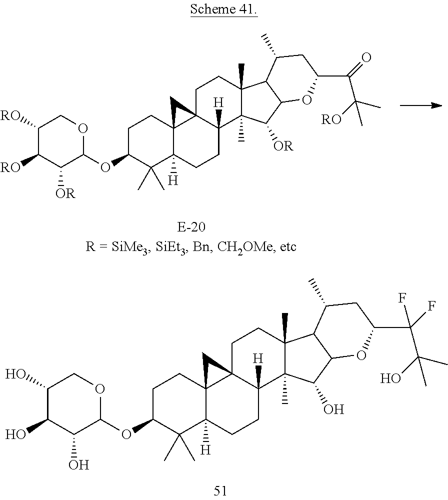

A 25-mL flask is charged with protected polyol E-20 (1 mmol) dissolved in 10 mL of dichloromethane. A nucleophilic fluorinating agent (3 mmol) is added and the resulting mixture is stirred at room temperature until TLC indicates complete consumption of the starting material. The reaction mixture is partitioned between water and dichloromethane and concentrated under reduced pressure. The residue is subjected to the appropriate conditions for removal of the hydroxyl protecting groups and purified by column chromatography to provide the difluoride 51.

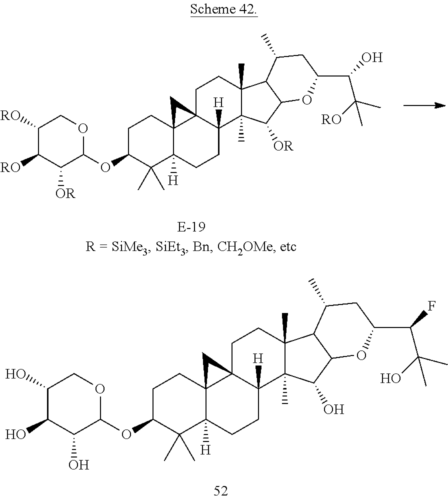

A 25-mL flask is charged with protected polyol E-19 (1 mmol) dissolved in 10 mL of dichloromethane and cooled to −20° C. A nucleophilic fluorinating agent (1.5 mmol) is added and the reaction mixture is allowed to warm to room temperature and stirred until TLC indicates complete consumption of the starting material. The resulting mixture is partitioned between water and dichloromethane and concentrated under reduced pressure. The residue is subjected to the appropriate conditions for removal of the hydroxyl protecting groups and purified by column chromatography to provide the difluoride 52.

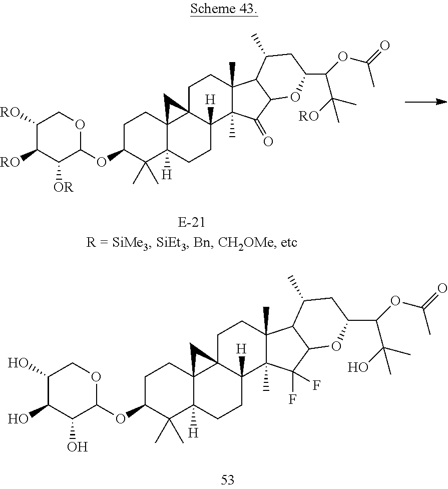

A 25-mL flask is charged with protected polyol E-21 (1 mmol) dissolved in 10 mL of dichloromethane. A nucleophilic fluorinating agent (3 mmol) is added and the reaction mixture is stirred at room temperature until TLC indicates complete consumption of the starting material. The resulting mixture is partitioned between water and dichloromethane and concentrated under reduced pressure. The residue is subjected to the appropriate conditions for removal of the hydroxyl protecting groups and purified by column chromatography to provide the difluoride 53.

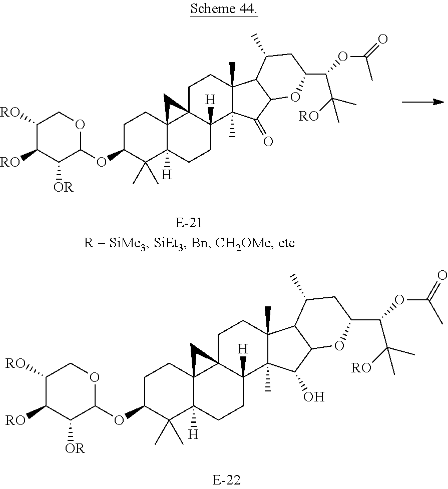

A 25-mL flask is charged with protected polyol E-22 (1 mmol) dissolved in 10 mL of ethyl acetate. Sodium borohydride (0.38 g, 1 mmol) is added and the resulting mixture is stirred at room temperature until TLC indicates complete consumption of the starting material and then concentrated under reduced pressure. The residue is diluted with dichloromethane and added dropwise to a 0° C. solution of 5% aqueous citric acid. The organic phase is separated and concentrated and the residue purified by column chromatography to provide the alcohol E-23.

### Example 3

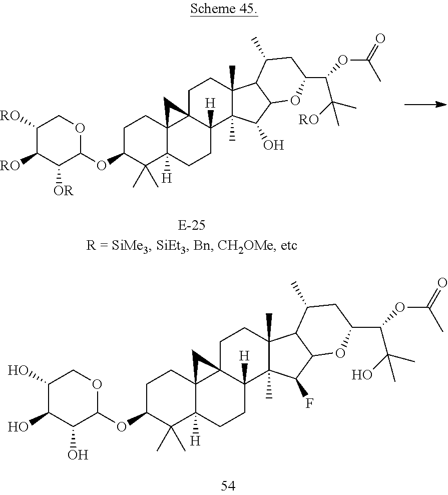

A 25-mL flask is charged with protected polyol E-22 (1 mmol) dissolved in 10 mL of dichloromethane and cooled to −20° C. A nucleophilic fluorinating agent (1.5 mmol) is added and the reaction mixture is allowed to warm to room temperature and stirred until TLC indicates complete consumption of the starting material. The resulting mixture is partitioned between water and dichloromethane and concentrated under reduced pressure. The residue is subjected to the appropriate conditions for removal of the hydroxyl protecting groups and purified by column chromatography to provide the difluoride 54.

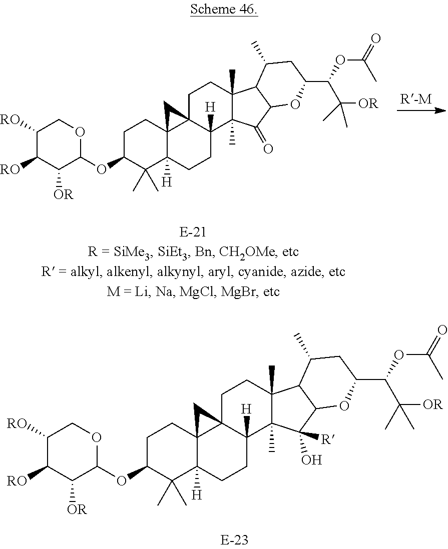

A 25-mL flask is charged with protected polyol E-21 (1 mmol) in 10 mL of DMF or other polar aprotic solvent and cooled at −50° C. A solution of the nucleophile (3 mmol) is added dropwise and the reaction mixture is allowed to warm to room temperature and stirred until TLC indicates complete consumption of the starting material and partitioned between water and organic solvent. The organic phase is separated and concentrated. If required due to concomitant deacetylation, the residue is dissolved in 10 mL of dichloromethane DMAP (0.18 g, 1.1 mmol) is added, followed by 0.10 mL acetic anhydride (110 mg, 1.1 mmol, 1.1 equiv) and the reaction mixture is stirred at room temperature until TLC indicates complete consumption of the starting material. The resulting mixture is partitioned between water and dichloromethane and the organic phase is concentrated. In either event the crude product is purified by column chromatography on silica gel to provide the acetate E-23.

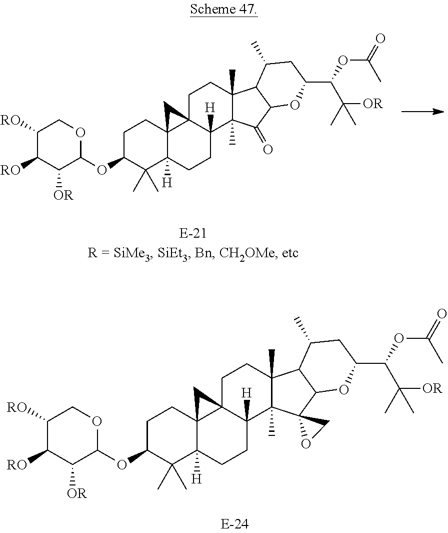

A 10 mL flask is charged with trimethylsulfoxonium bromide (0.210 g, 1.2 mmol, 1.2 equiv) and the protected ketone E-21 in 10 mL of DMSO and cooled at 0° C. while a potassium tert-butoxide (0.130 g, 1.2 mmol, 1.2 equiv) was added. The resulting mixture is stirred at while warming slowly to room temperature. Stirring is continued until TLC indicates complete consumption of the starting material. The resulting mixture is partitioned between water and ether. The organic phase is concentrated and purified by column chromatography on silica gel to provide the epoxide E-24.

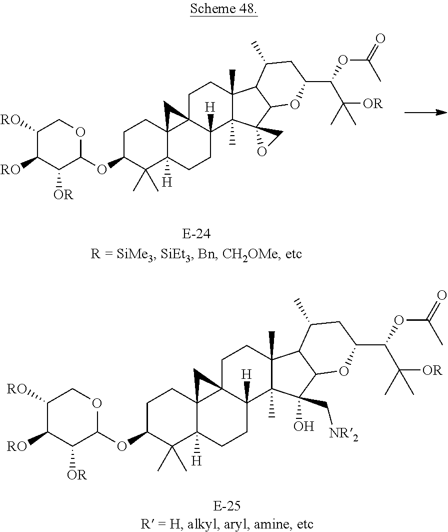

A 10-mL flask is charged with epoxide E-24 (1 mmol) in 1 mL of DMF and an amine (2 mmol) is added. The reaction mixture is heated at reflux until TLC indicates complete consumption of the starting material, and then partitioned between dichloromethane and water. The organic phase is concentrated and the residue is purified by silica gel chromatography to provide the amino alcohol E-25.

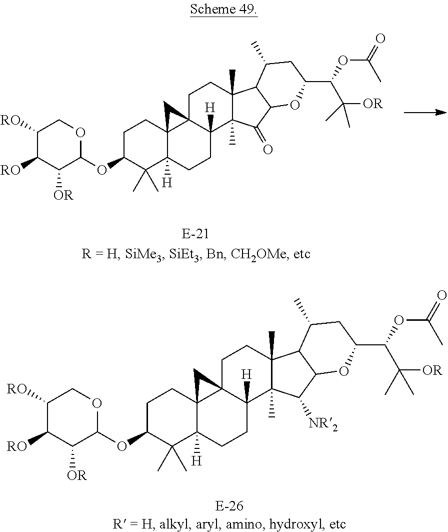

A 25-mL flask is charged with a solution of the polyol E-21 (1 mmol) in 8 mL of THF and 2 mL of THF and the mixture is stirred at room temperature while amine (20 mmol) and sodium cyanoborohydride (154 mg, 2 mmol, 2 equiv) is added. Stirring is continued and additional sodium cyanoborohydride (77 mg, 1 mmol) is added daily until TLC indicates complete consumption of the starting material. The reaction mixture is partitioned between ether and water, the organic phase is concentrated, and the residue is purified by column chromatography on silica gel to provide the amine E-26.

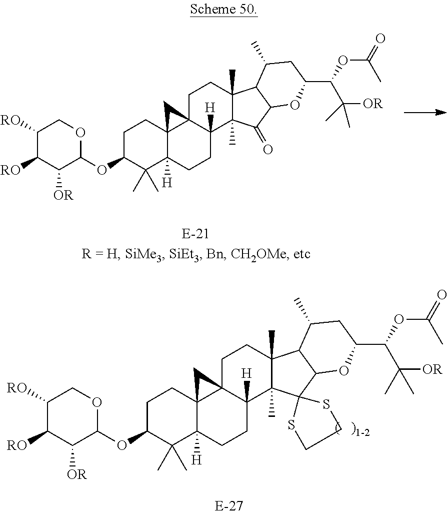

A 25-mL flask is charged with a solution of the polyol E-21 (1 mmol) and dithiol (10 mmol) in 10 mL of dichloromethane and cooled at 0° C. while a solution of boron trifluoride etherate (1 mmol) was added. The resulting mixture was stirred at room temperature until TLC indicates the complete consumption of starting material. The reaction mixture is partitioned between ether and water, the organic phase is concentrated, and the residue is purified by column chromatography on silica gel to provide the amine E-27.

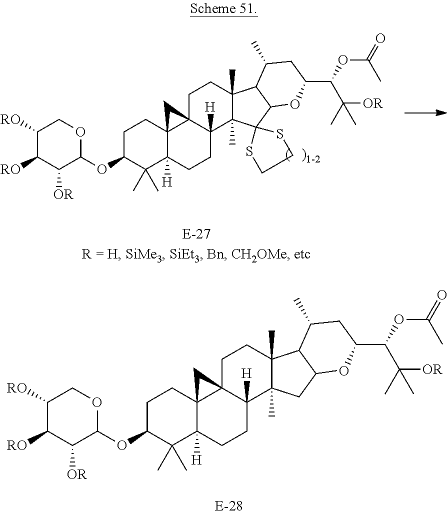

A 25-mL flask is charged with protected polyol E-27 (1 mmol) in 10 mL of ethanol, and Raney Nickel (220 mg, 4 mmol, 4 equiv) is added. The resulting mixture is heated at reflux until TLC indicates complete consumption of starting material, and is poured into 5% aqueous citric acid. The resulting mixture is partitioned between water and ether and the organic phase is concentrated. If required due to concomitant deacetylation, the residue is dissolved in 10 mL of dichloromethane, DMAP (0.18 g, 1.1 mmol) is added followed by 0.10 mL acetic anhydride (110 mg, 1.1 mmol, 1.1 equiv) and the reaction mixture is stirred at room temperature until TLC indicates complete consumption of the starting material. The resulting mixture is partitioned between water and dichloromethane and the organic phase is concentrated. In either event the crude product is purified by column chromatography on silica gel to provide the acetate E-28.

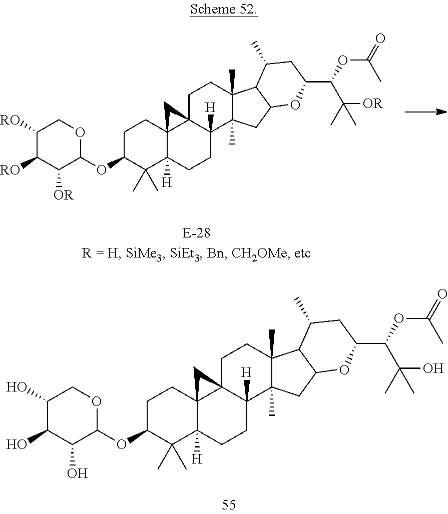

A 25-mL flask is charged with protected polyol E-28 (1 mmol) and subjected to the appropriate conditions for the removal of the hydroxyl protecting groups. The resulting mixture is partitioned between water and organic solvent, the organic phase is concentrated and the residue is purified by column chromatography on silica gel to provide the polyol 55.

### Example 4

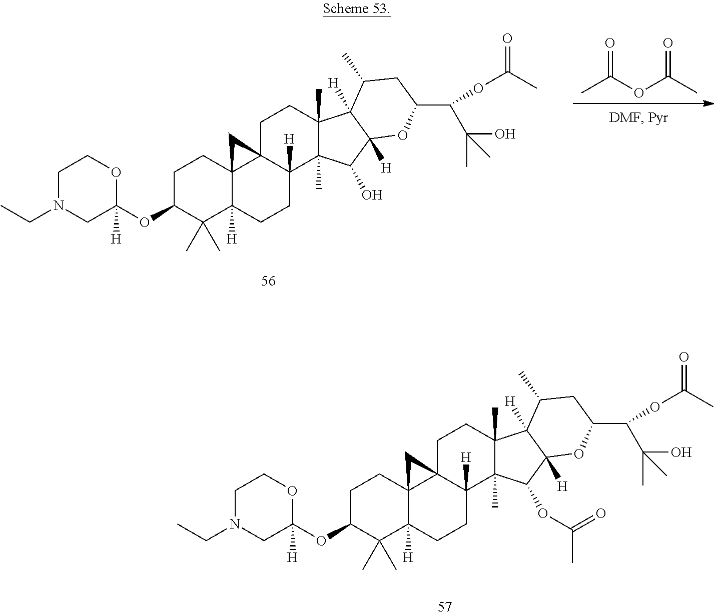

Compound 56 (23 mg) and DMAP (1 mg) were dissolved in dry DMF (1 mL) under argon. To this solution was added pyridine (200 μL) and acetic anhydride (100 μL) and the mixture was allowed to stir for 2 hours at room temperature. The solvent was then removed in vacuo and the resulting residue was purified by silica gel chromatography (0-5% MeOH/CH₂Cl₂) to give compound 57. m/z=688 (M⁺+H).

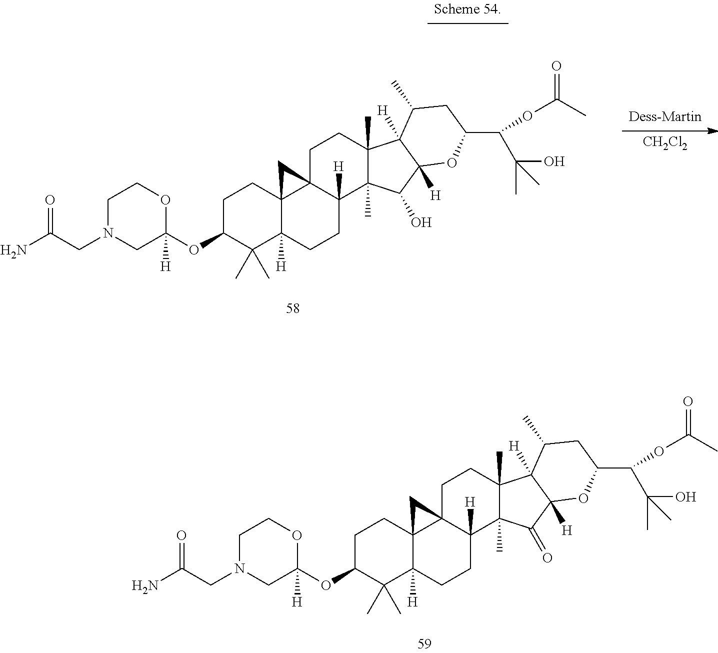

To compound 58 (4 mg) and Dess-Martin periodinane (4 mg) was added CH₂Cl₂ (1 mL) and stirred at room temperature for 3 hours. The solution was then passed through a plug of celite and washed with excess CH₂Cl₂. The solvent was then removed in vacuo and the resulting residue was purified by silica gel chromatography (0-5% MeOH/CH₂Cl₂) to give compound 59. m/z=695 (M⁺+Na).

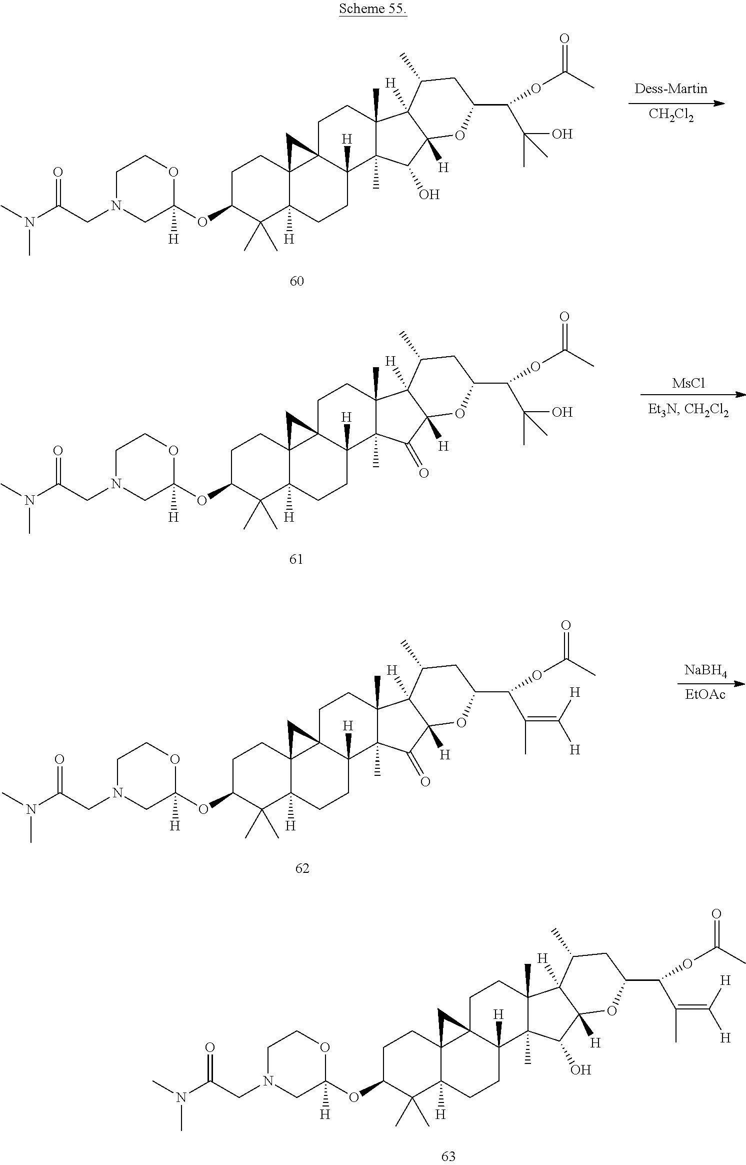

To compound 60 (25 mg) and Dess-Martin periodinane (49 mg) was added CH₂Cl₂ (10 mL) and the solution was stirred at room temperature for 1 hour. The solution was then passed through a plug of celite and washed with excess CH₂Cl₂. The solvent was then removed in vacuo and the resulting residue was purified by silica gel chromatography (5% MeOH/CH₂Cl₂) to give compound 61 as a white solid (m/z=724 (M⁺+Na)). To this was added DMAP (1 mg) and the solids were dissolved in CH₂Cl₂ (25 mL). Triethylamine (124 μL) was then added and the solution was cooled to 0° C. under an argon atmosphere. Next, mesyl chloride (33 μL) was added to the solution and the temperature was slowly raised to room temperature over 1 hour and the reaction was then allowed to stir overnight. The solvent was then removed in vacuo and the residue was purified by silica gel chromatography. The purified intermediate was then dissolved in EtOAc (6 mL). NaBH₄ (25 mg) was then added and the solution was ultra-sonicated for 3 minutes and the reaction mixture was allowed to stir overnight at room temperature. The solvent was removed in vacuo and the residue was re-dissolved in CH₂Cl₂ (15 mL). The solution was then added drop wise to an ice chilled aqueous solution of 5% citric acid (10 mL) in a separating funnel which caused vigorous bubbling. Once all of the solution had been added and bubbling had ceased, the organic layer was separated. The resulting residue was purified by silica gel chromatography (0-5% MeOH/CH₂Cl₂) to give compound 63. m/z=707 (M⁺+Na).

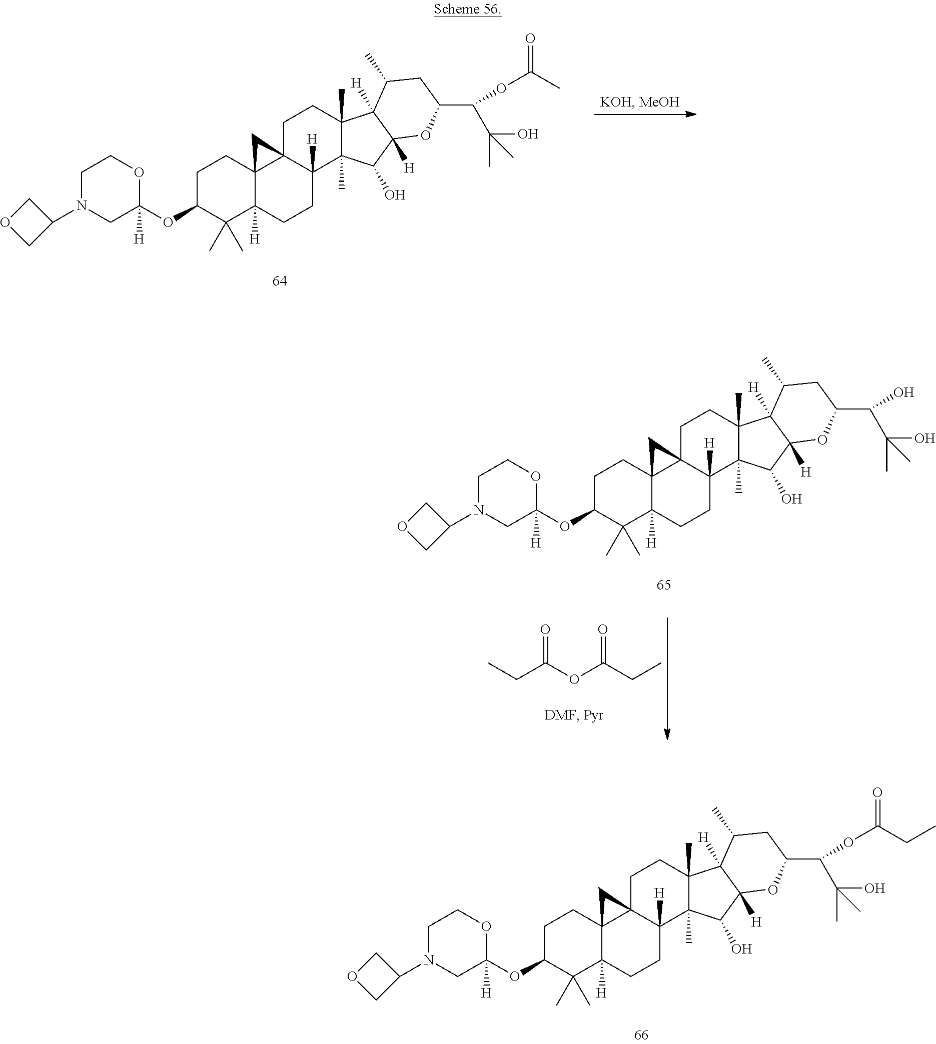

Compound 64 (31 mg) was dissolved in MeOH (3 mL) and treated with 60 μL of methanolic KOH (0.5 g in MeOH (2 mL)) for 2 hours. The solvent was then removed in vacuo and the residue was purified by silica gel chromatography (5% MeOH/CH₂Cl₂) to give compound 65 (m/z=655 (M⁺+Na). The product was then dissolved in dry DMF (4 mL) and DMAP (3 mg) and pyridine (15 μL) were added. To this solution was added propionic anhydride (6 μL) and the reaction was allowed to stir for 2 days at room temperature. Additional 10 μL amounts of pyridine and propionic anhydride were added for 3 consecutive days until the reaction was complete as indicated by LCMS analysis. The solvent was then removed in vacuo and the resulting residue was purified by silica gel chromatography (0-5% MeOH/CH₂Cl₂) to give compound 66. m/z=710 (M⁺+Na).

### Example 5

### General Procedure for Reductive Aminations Using Compound 67

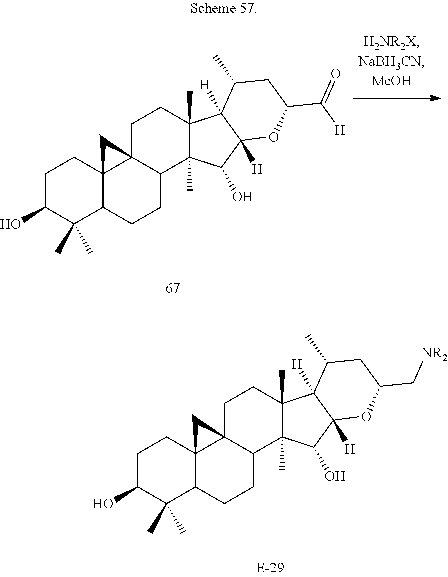

A 25-mL flask is charged with aldehyde 67 (1 mmol) in 10 mL of methanol or other polar protic solvent and stirred at 25° C. while the amine salt (2 mmol) is added. The resulting mixture is stirred at room temperature for 16 h. NaBH₃CN is added in small portions (1-2 mmol each) spaced 8-16 h apart until TLC or LCMS indicates complete consumption of the starting material. The reaction mixture is concentrated and filtered through a plug of silica gel. Purification by chromatography on silica gel yields the desired amine E-29.

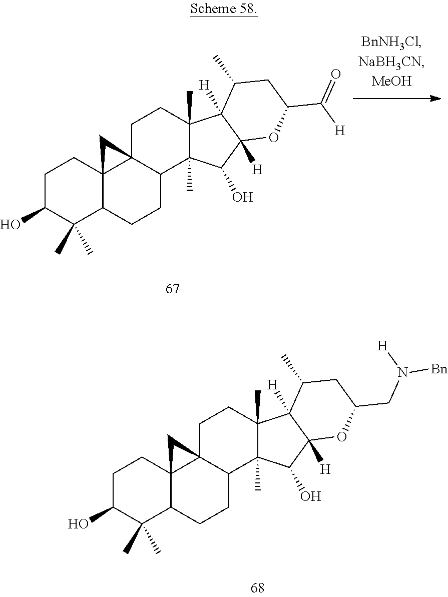

A 25-mL flask was charged with aldehyde 67 (50 mg, 0.12 mmol) in 4 mL of methanol and stirred at 25° C. while the benzylamine hydrochloride (30 mg, 0.21 mmol) was added. The resulting mixture was stirred at room temperature for 16 h. NaBH₃CN (10 mg, 0.16 mmol) was added, the mixture was stirred for 8 h, then additional NaBH₃CN (10 mg, 0.16 mmol) was added, and the mixture was stirred for 16 h. A final batch of NaBH₃CN (10 mg, 0.16 mmol) was added and the mixture was stirred for 8 h, then concentrated and filtered through a plug of silica gel. Purification by chromatography on silica gel yielded 44 mg of the desired amine 68.

LCMS (m/z): [M+H]⁺ 522.

Representative other amines prepared by this method include:

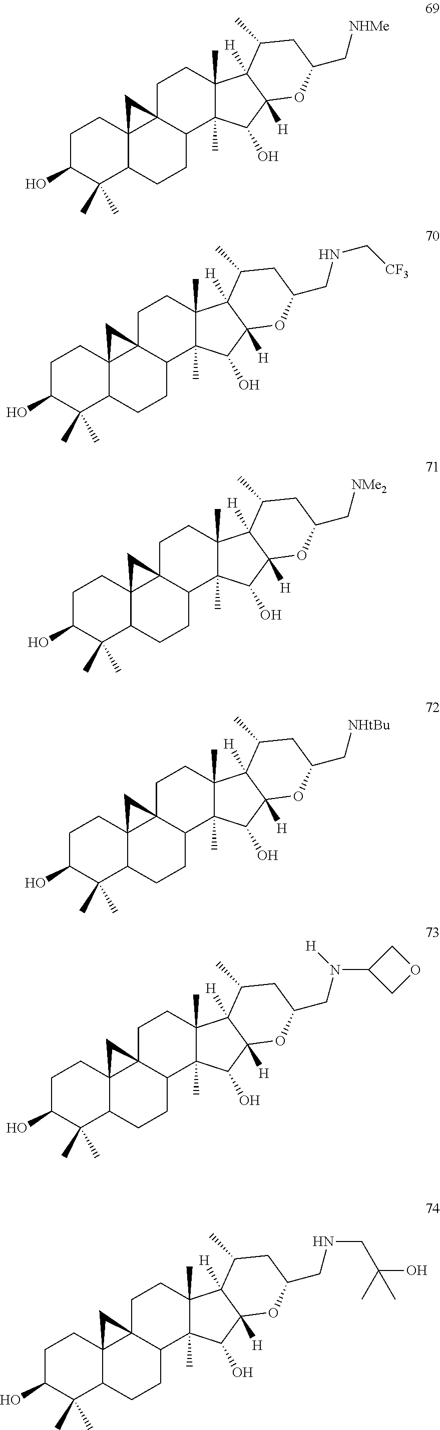

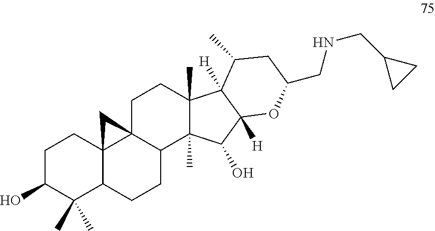

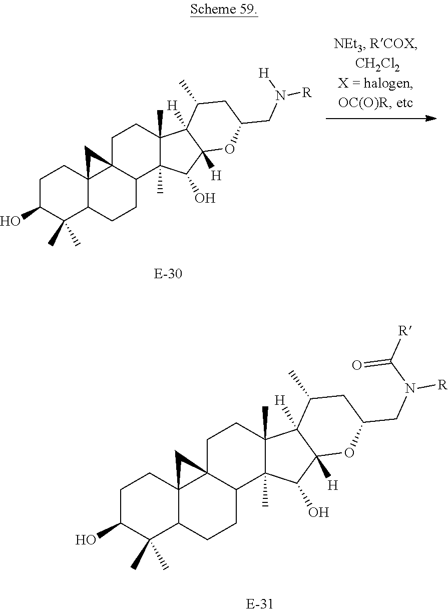

A 25-mL flask is charged with amine E-30 (1 mmol) and triethylamine (10 mmol) in 10 mL of CH₂Cl₂ and stirred at room temperature while an acylating agent (1.1 mmol) is added. The resultant mixture is stirred at 0-40° C. until TLC or LCMS indicates complete consumption of the starting material and then partitioned between water and organic solvent and concentrated. Purification by column chromatography on silica gel yields the desired amide E-31.

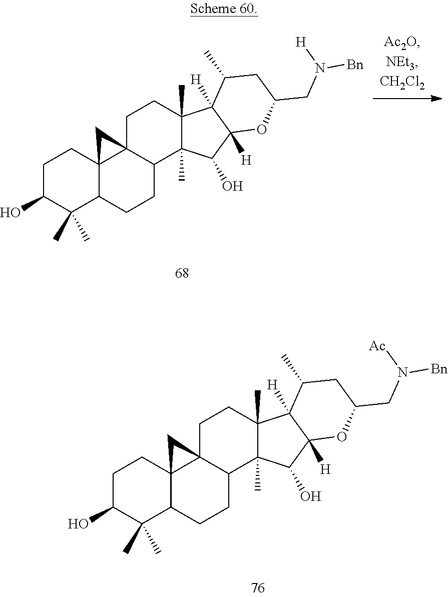

A 25-mL flask was charged with a benzyl amine 68 (32 mg, 0.061 mmol) and triethylamine (40 μL, 29 mg, 0.29 mmol) in 1 mL of CH₂Cl₂ and stirred at room temperature while an acetic anhydride (11 μL, 12 mg, 012 mmol) was added. The resulting mixture was stirred at room temperature for five hours and then partitioned between water and CH₂Cl₂ and concentrated. Purification by column chromatography on silica gel yields 35 mg of the desired amide 76. LCMS (m/z): [M+Na]⁺ 586.

Representative Amides Prepared in this Fashion Include:

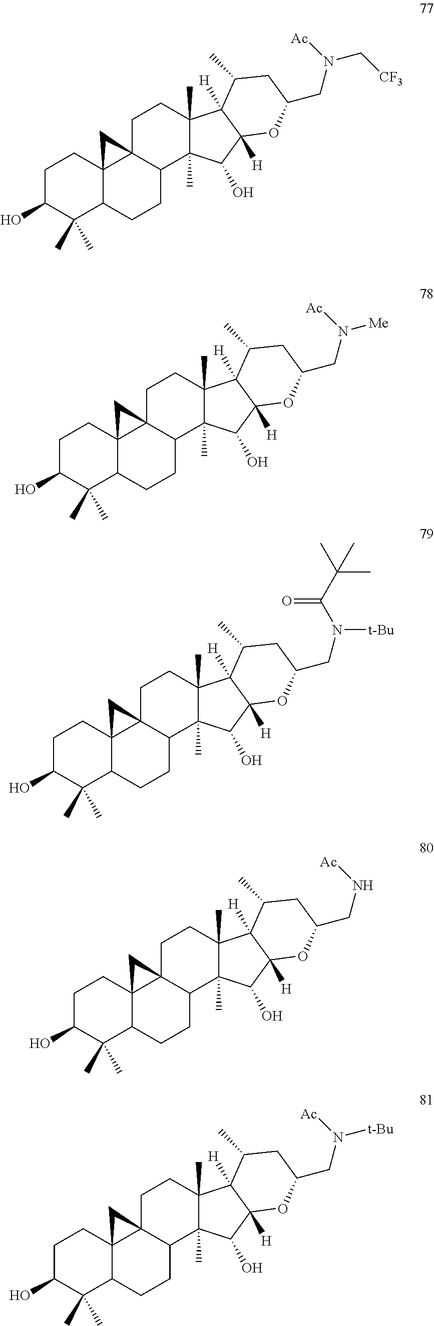

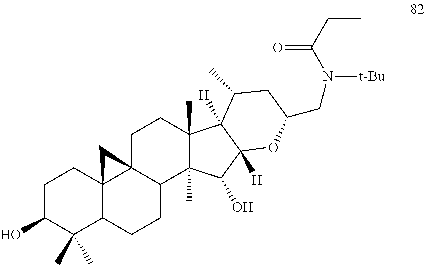

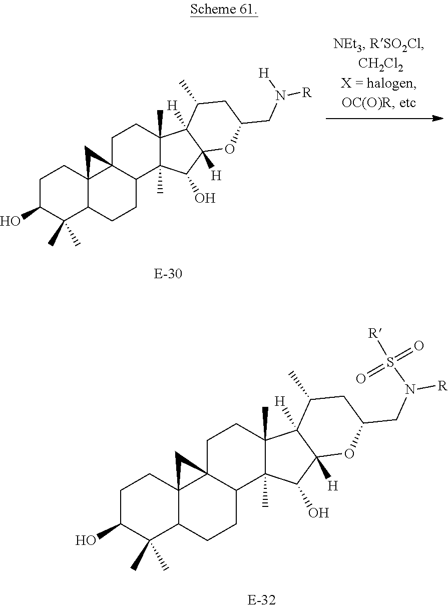

A 25-mL flask is charged with amine E-30 (1 mmol) and triethylamine (10 mmol) in 10 mL of CH₂Cl₂ and stirred at 0° C. while an sulfonyl chloride (1.1 mmol) is added. The resulting mixture is stirred at 0-40° C. until TLC or LCMS indicates complete consumption of the starting material and then partitioned between water and organic solvent. Purification by column chromatography on silica gel yields the desired sulfonamide E-32.

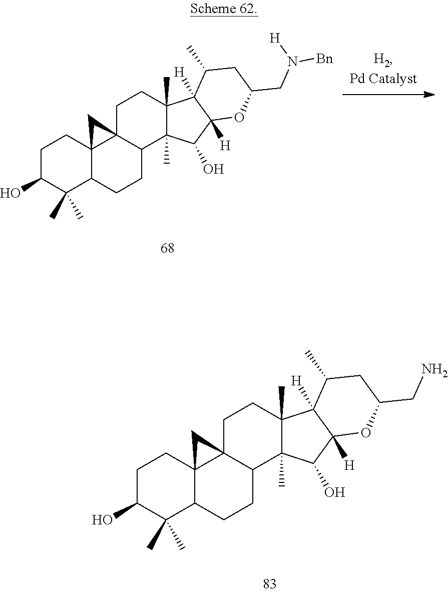

A 25-mL flask is charged with benzylamine 68 (111 mg, 0.213 mmol) in 10 mL of methanol and 1 mL of trifluoroacetic acid. Palladium hydroxide on carbon (40 mg, 10 wt % Pd, 0.038 mmol) is added and the reaction mixture is stirred under a hydrogen atmosphere (1 atm) for 7 days, with additional palladium hydroxide (40 mg, 10 wt % Pd, 0.038 mmol) added daily. The resulting mixture is filtered through a plug of celite and concentrated. Purification by column chromatography provides the desired amine 83. LCMS (m/z): [M+H]⁺ 432.

### Example 6

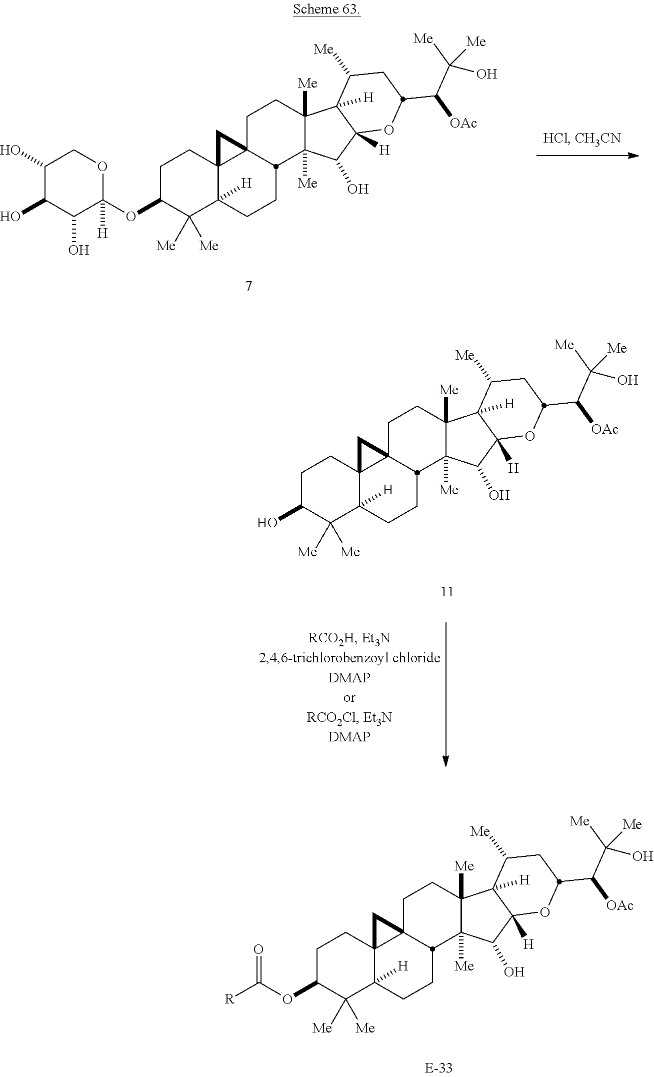

Compound 7 (627 mg, 0.942 mmol) was suspended in 40 mL CH₃CN and 10 mL conc. HCl was added. The solution was stirred for 1 h then carefully poured into 200 mL NaHCO₃ (saturated aq). The aqueous layer was extracted twice with CH₂Cl₂, the combined extracts dried with Na₂CO₃, and the solvent removed. The residue was purified by flash chromatography (25 g column, 10-100% ethyl acetate in hexanes) to afford 352 mg (70 aglycone 11 as a white solid.

Procedure 1

2,4,6-Trichlorobenzoyl chloride (2.00 equiv) was added to a solution of 11 (1.00 equiv), carboxylic acid (1.05 equiv) and triethylamine (5.00 equiv) in CH₂Cl₂ at room temperature. The solution was allowed to stir for 1 h then DMAP (1.20 equiv) was added and the solution as allowed to stir for an additional 30 minutes. The resulting ester solution was purified by Biotage flash chromatography.

Procedure 2

Acid chloride (1.05 equiv) was added to a solution of 11 (1.00 equiv) and triethylamine (5.00 equiv) in CH₂Cl₂ room temperature. DMAP (1.20 equiv) was added and the solution was allowed to stir for 30 minutes. Additional acid chloride was added if TLC or LC/MS indicated that a significant amount of starting material remained. The resulting ester E-33 solution was purified by Biotage flash chromatography.

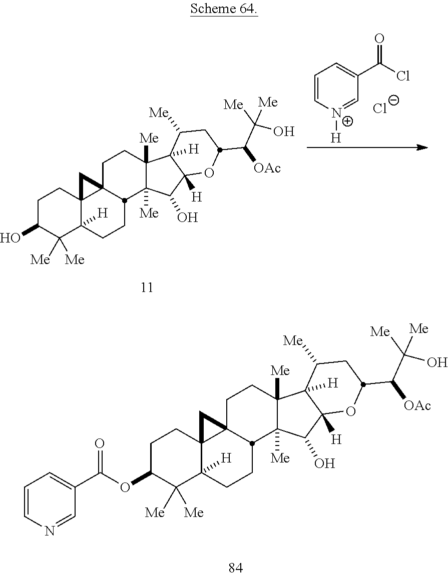

Nicotinyl chloride hydrochloride (23.3 mg, 0.131 mmol) was added to a solution of 11 (60 mg, 0.113 mmol) and triethylamine (79 μL, 0.565 mmol) in CH₂Cl₂ at room temperature. DMAP (17 mg, 0.136 mmol) was added and the solution was allowed to stir for 30 minutes. Additional nicotinyl chloride (6.0 mg, 0.034 mmol) was added, the solution stirred an additional 18 h, and again additional nicotinyl chloride (13.0 mg, 0.073 mmol) was added and the solution stirred 30 minutes. One last portion of nicotinyl chloride (5.0 mg, 0.028 mmol) was added and the solution stirred 30 minutes. The resulting ester solution was purified by Biotage flash chromatography (0-100% ethyl acetate/hexanes) to give ester 84 as a white solid (59 mg, 82%). MS (m/z) 598.4 (M+Na)⁺.

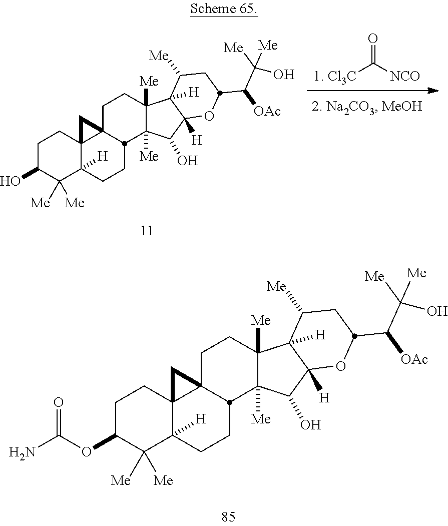

Trichloroacetylisocyanate (10.7 μL, 0.0900 mmol) was added to a solution of alcohol 11 (0.0750 mmol) at room temperature in CH₂Cl₂ (1 mL) under nitrogen and allowed to stir for 10 minutes. The resulting solution was purified by Biotage flash chromatography (10 g column, 15-100% ethyl acetate/hexanes) to give the trichloroacetyl carbamate. The carbamate was dissolved in 5 mL of methanol and 10 mg of Na₂CO₃ was added. The solution was stirred for 25 minutes then partitioned between CH₂Cl₂ and 1 N HCl. The organic layer was dried over Na₂SO₄ and the solvent was removed. The residue was purified by Biotage flash chromatography (10 g column) to give the desired primary carbamate 85. MS (m/z) 598.4 (M+Na)⁺.

### Example 7

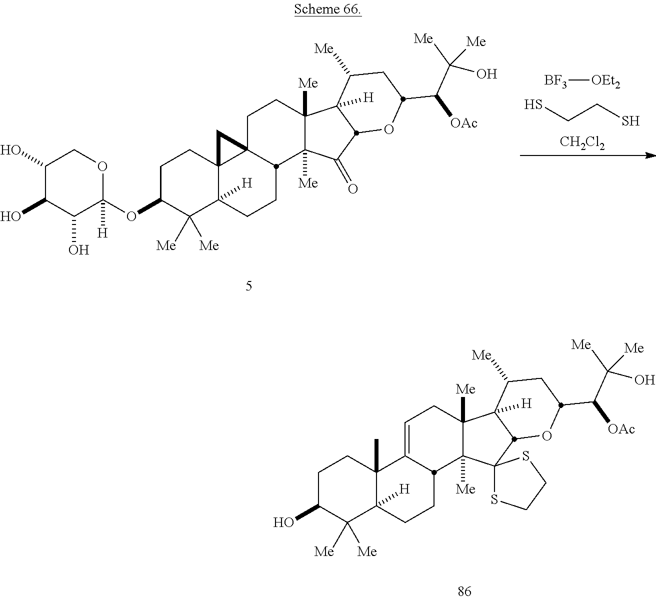

A 10-mL flask is charged with glycoside 5 (0.150 g, 0.227 mmol) and ethanedithiol (0.4 mL, 0.45 g, 4.8 mmol) in 4 mL CH₂Cl₂ and stirred at room temperature while was boron trifluoride etherate (0.2 mL, 0.23 g, 1.62 mmol) is added. The resulting mixture is stirred for 48 h, then partitioned between water and ether and concentrated. Purification by column chromatography on silica gel yields the desired alkene 86.

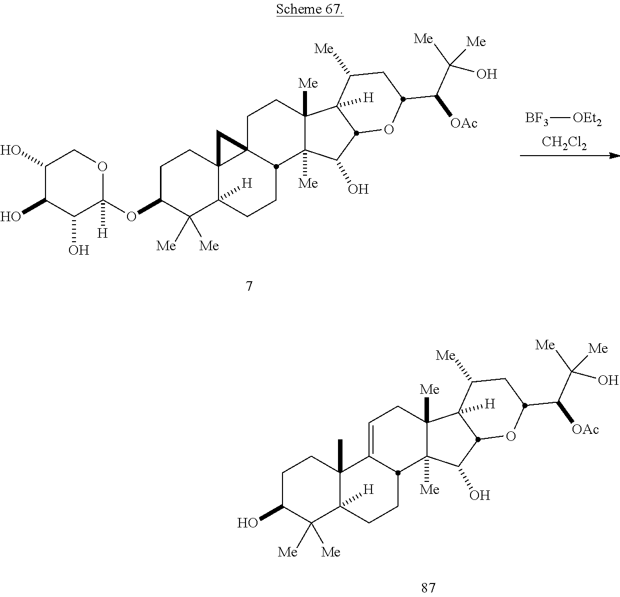

A 10-mL flask is charged with glycoside 7 (0.100 g, 0.150 mmol) in 3 mL CH₂Cl₂ and stirred at room temperature while was boron trifluoride etherate (0.1 mL, 0.12 g, 0.81 mmol) is added. The resulting mixture is stirred for 24 h, then partitioned between water and ether and concentrated. Purification by column chromatography on silica gel yields the desired alkene 87.

### Example 8

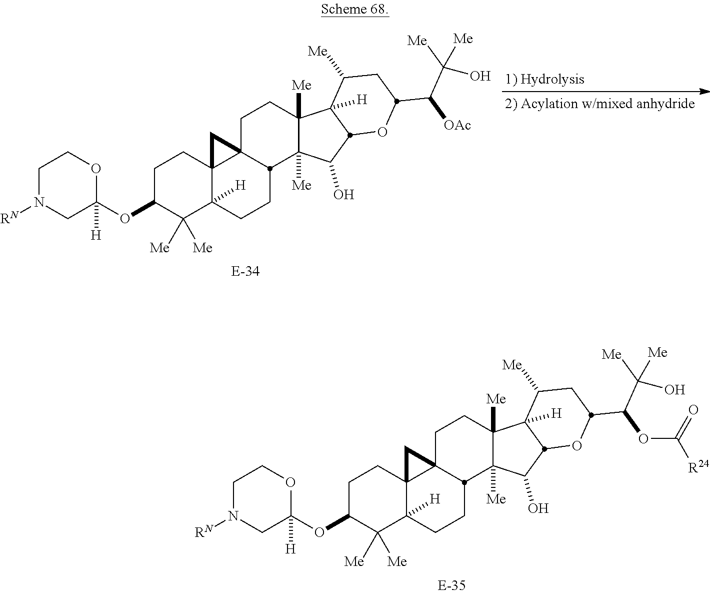

Depicted in Scheme 68 above is the transformation of acetate E-34 at C-24 to an analog thereof, accessible via hydrolysis of the C-24 acetate followed by acylation with an appropriate mixed anhydride. Exemplary R²⁴ groups include, but are not limited to alkyl groups (e.g., methyl, ethyl, propyl, butyl, pentyl, hexyl, etc.) and cycloalkyl groups (e.g., cyclopropyl, cyclobutyl, cyclopentyl, cyclohexyl, etc.). Exemplary R^{N} substitutents include, but are not limited to, optionally substituted cyclic and acyclic alkyl and heteroalkyl groups (e.g., THF, THP, oxetanes, alkyl amides, etc.). Specific conditions are as described in examples above and herein.

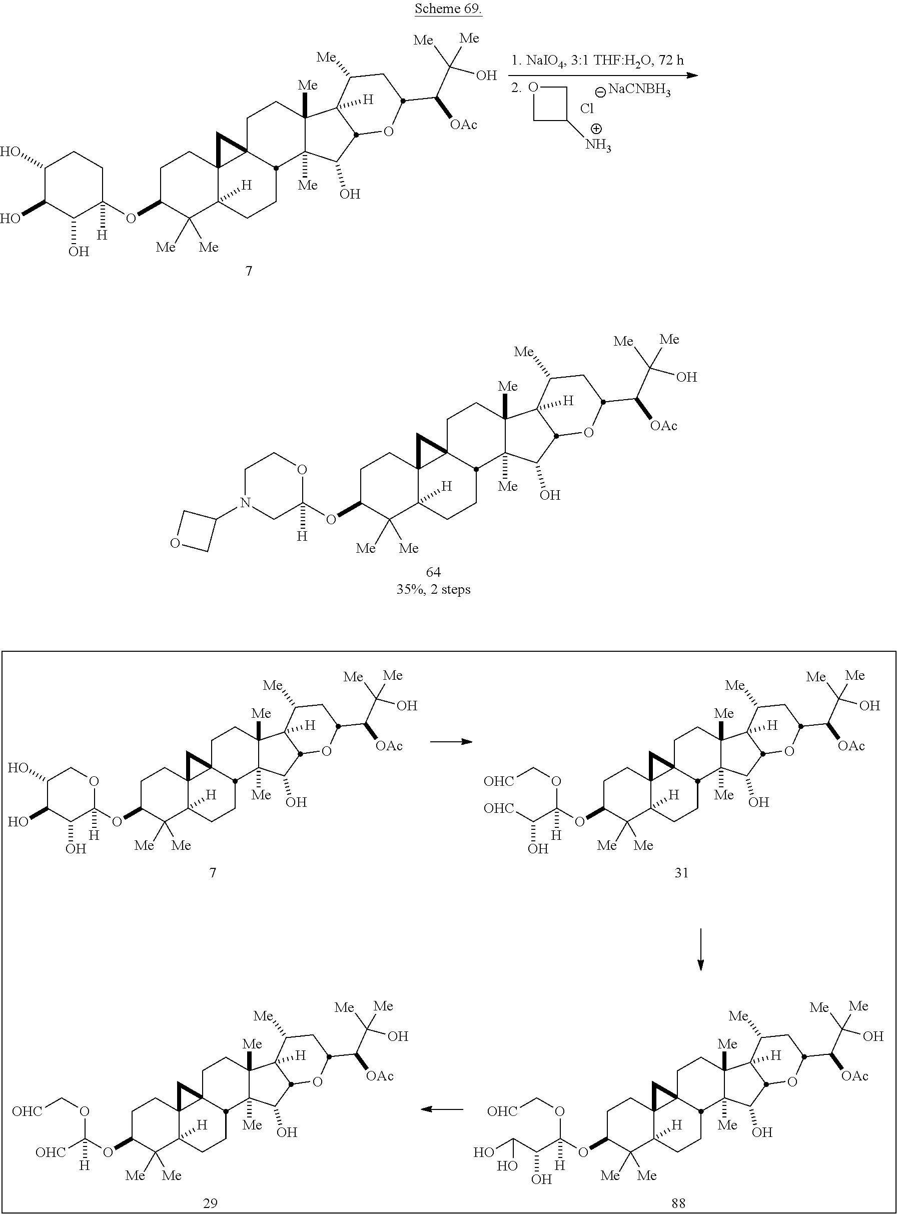

Scheme 69 above depicts an exemplary synthesis of compound 64 from compound 7. Compound 7 undergoes oxidative cleavage using sodium periodate in a 3:1 solution of THF:H₂O for 72 h to afford dialdehyde 29. Reductive amination of dialdehyde 29 affords the oxetane-bearing morpholino analog 64 in a 35% yield over two steps.

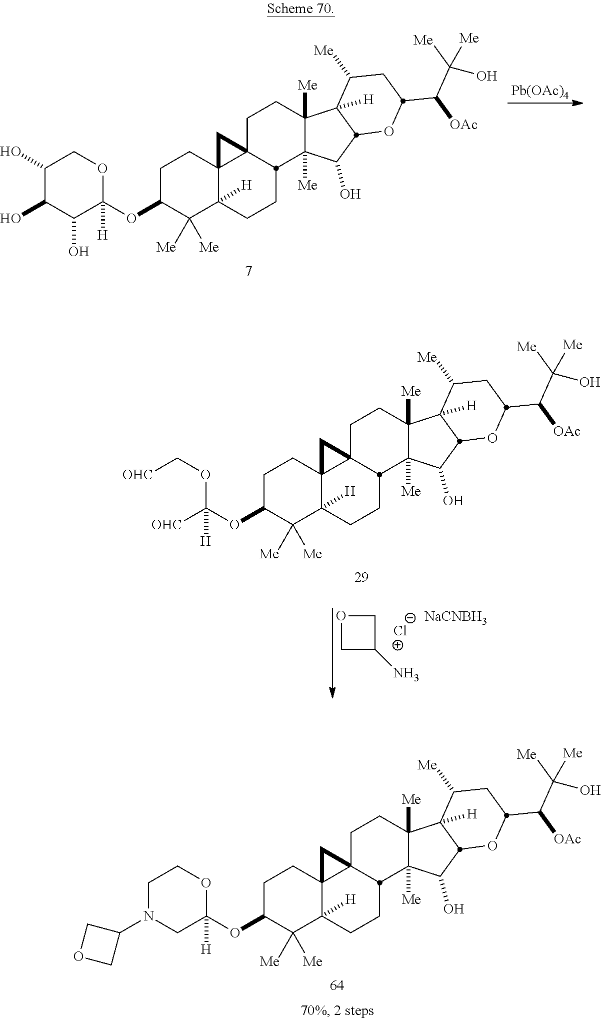

Alternatively, and as depicted in Scheme 70 above, compound 64 can be synthesized from compound 7 via oxidative cleavage of the diol moiety of 7 using lead tetraacetate to yield dialdehyde 29. Reductive amination of dialdehyde 29 affords oxetane-bearing morpholine analog 64 in a 70% yield over two steps.

### Example 9

TABLE 1
Compounds

| | |
|---|---|
| 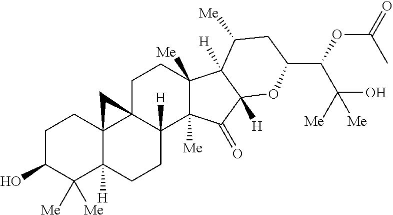 | 89 |
| 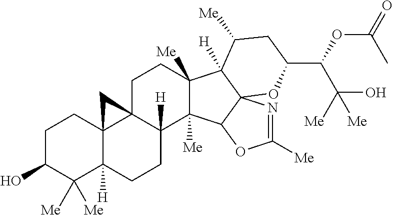 | 90 |
| 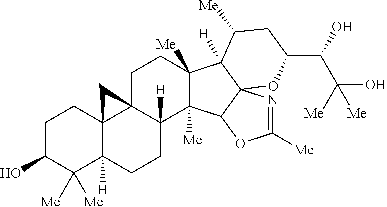 | 91 |
| 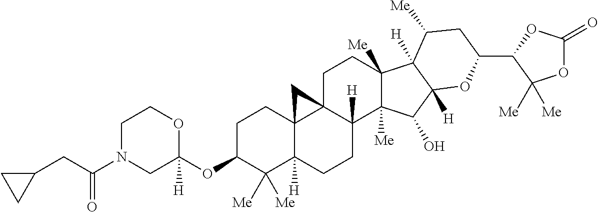 | 92 |
| 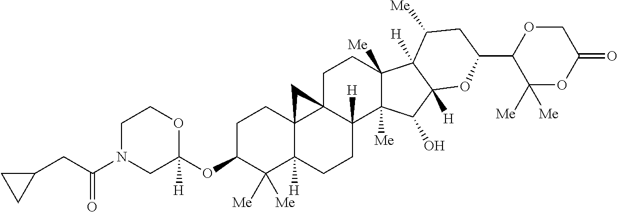 | 93 |
| 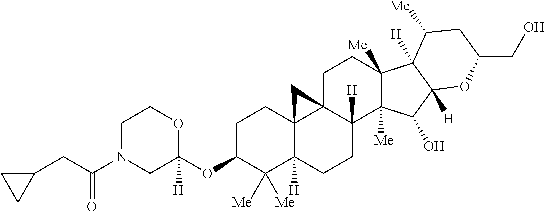 | 94 |
| 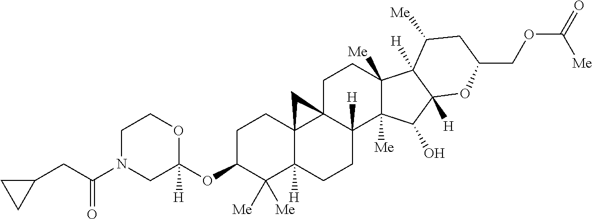 | 95 |
| 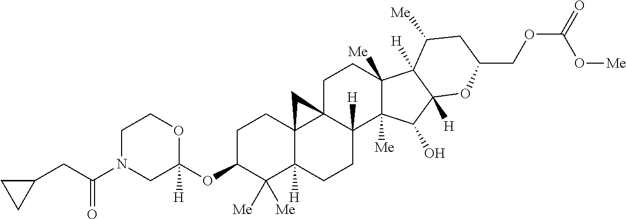 | 96 |
| 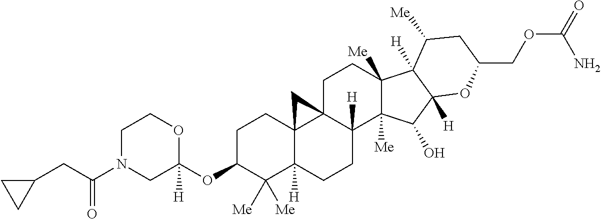 | 97 |
| 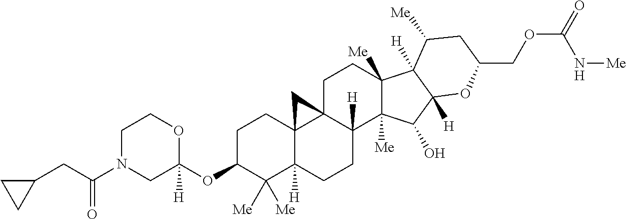 | 98 |
| 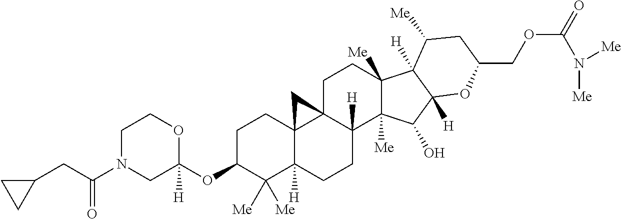 | 99 |
| 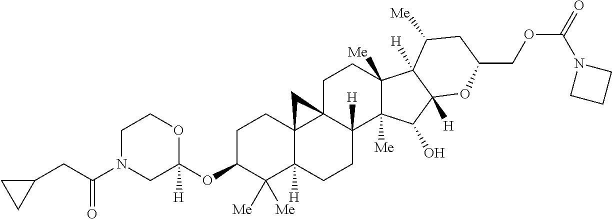 | 100 |
| 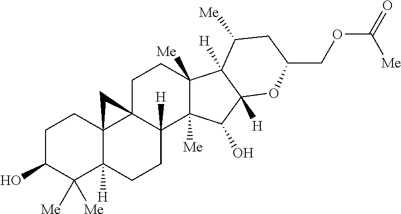 | 101 |
| 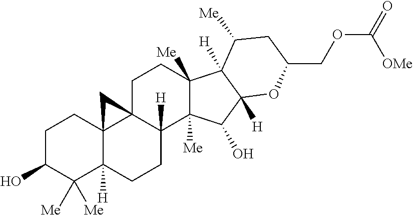 | 102 |
| 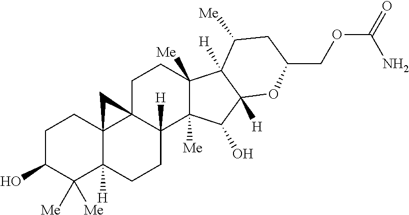 | 103 |
| 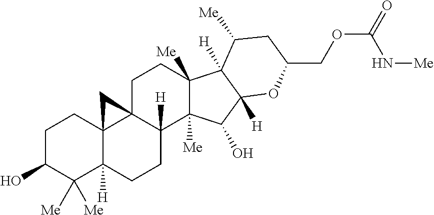 | 104 |
| 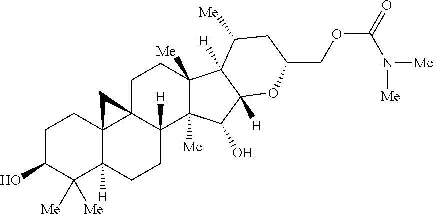 | 105 |
| 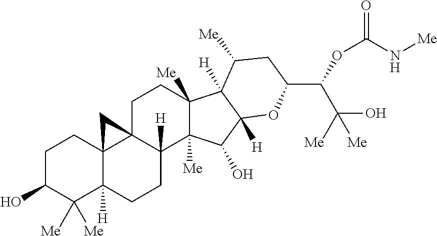 | 106 |
| 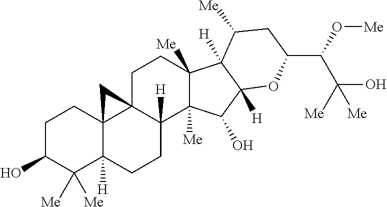 | 28 |
| 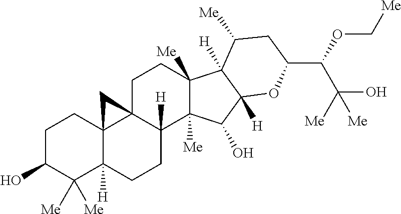 | 107 |
| 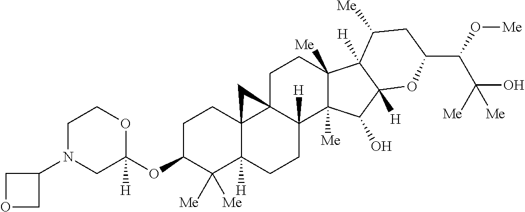 | 108 |
| 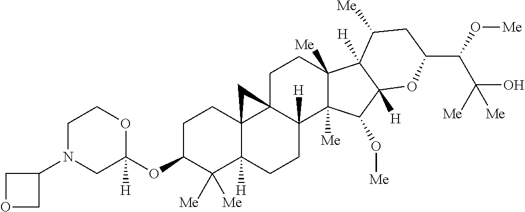 | 109 |
| 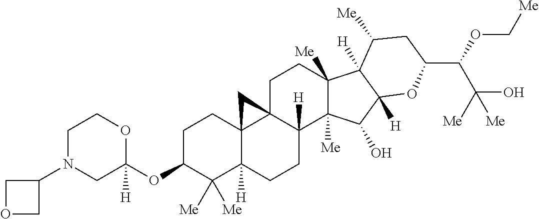 | 110 |
| 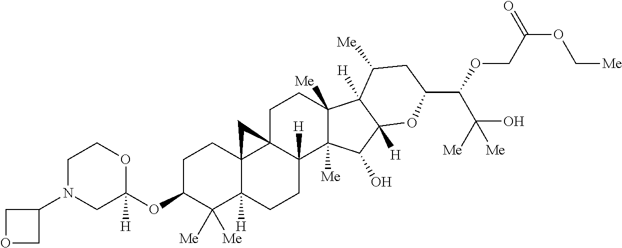 | 111 |
| 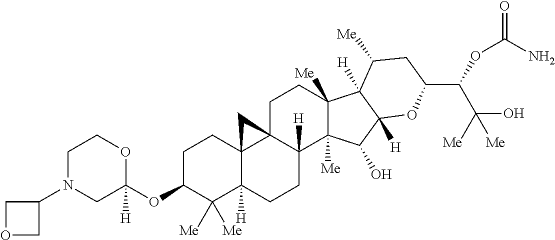 | 112 |

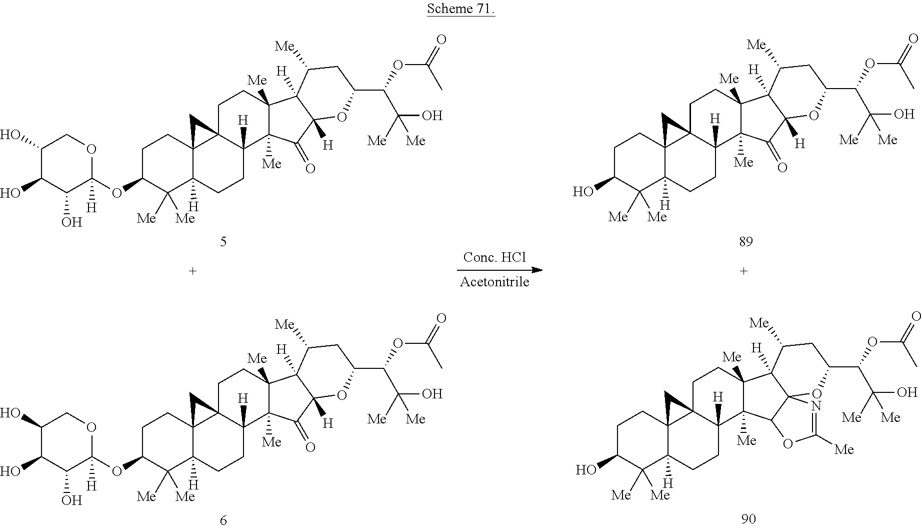

A mixture of compounds 5 and 6 (2.99 g) was dissolved in ACN (40 mL) and conc. HCl (10 mL) and stirred at RT for 1.5 h, whereupon it was diluted in CH₂Cl₂ (150 mL) and washed with aqueous NaHCO₃ until the aqueous phase remained basic. The organic layer was separated, the solvent was removed in vacuo, and the residue was purified by silica gel chromatography (2-7% MeOH/CH₂Cl₂) to separately give compounds 89 [1.75 g, m/z=553 (M⁺+Na)] and 90 [0.17 g, m/z=572 (M⁺+H)].

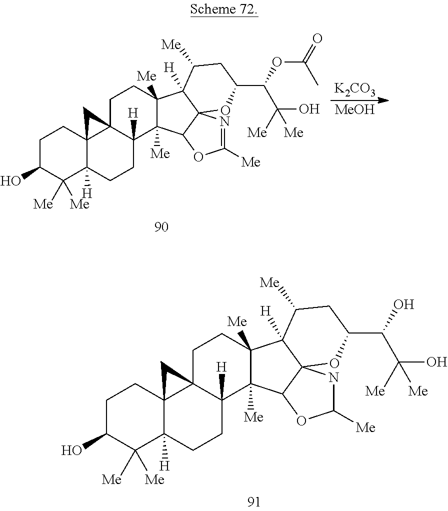

Compound 90 (33 mg) was dissolved in MeOH (10 mL) and K₂CO₃ (40 mg) was added. The solution was allowed to stir overnight and the solvent was removed in vacuo and the residue was dissolved in CH₂Cl₂ (20 mL) and washed twice with H₂O (5 mL). The organic layer was removed in vacuo and the product was purified by silica gel chromatography (5% MeOH/CH₂Cl₂) to give compound 91 [m/z=530, (M⁺+H)].

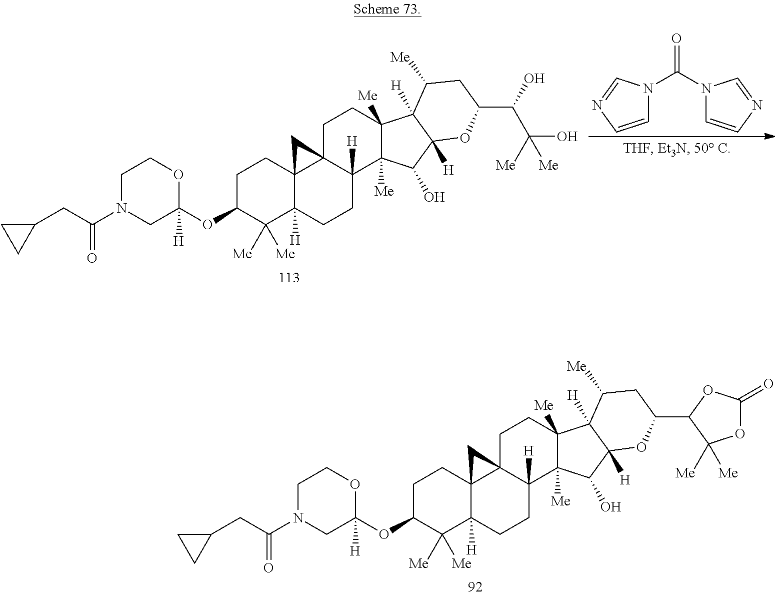

Compound 113 (25 mg) and N,N-carbonyldiimidazole (7.2 mg) was dissolved in THF (3 mL). Et₃N (52 μL) was then added and the solution was stirred overnight at 50° C. The solvent was removed in vacuo and the product was purified by C18 chromatography (40-90% ACN/H₂O (0.1% HCO₂H)) to give compound 92 [m/z=706 (M⁺+Na)].

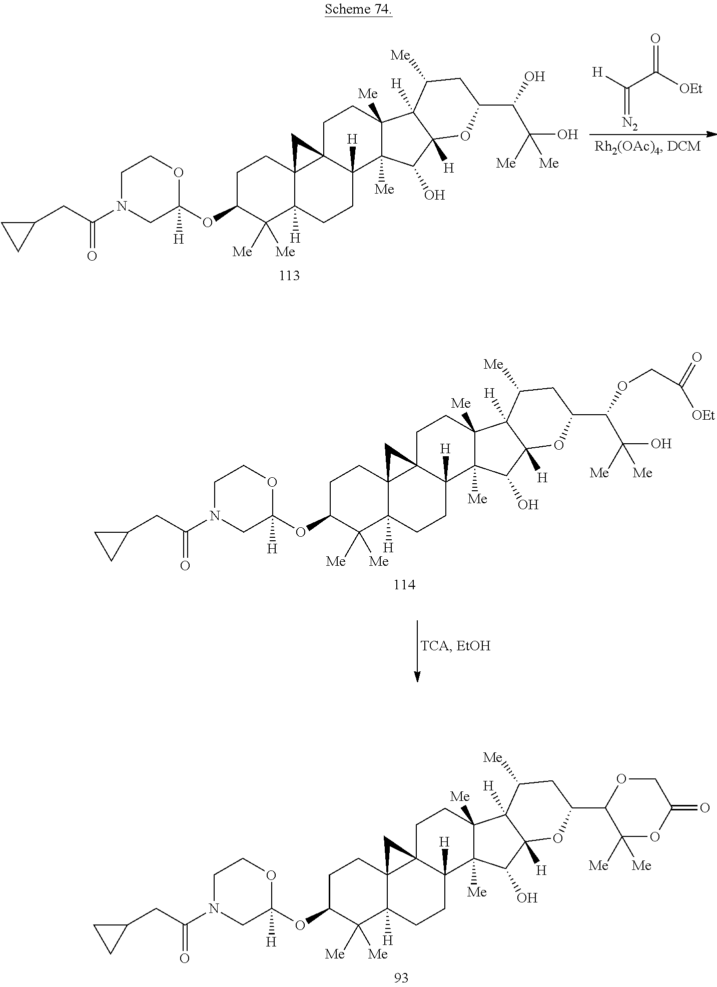

Compound 113 was dissolved in CH₂Cl₂ (3 mL) and ethyl diazoacetone (3.5 μL) was added. To the stirred solution was added rhodium (II) acetate (1.4 mg) and the solution was allowed to stir for 3 h. The solution was then diluted in CH₂Cl₂ (10 mL) and washed with H₂O (5 mL). The solvent was then removed and the residue was dissolved in EtOH (20 mL) and TCA (2 mg) was added. The solution was stirred for 30 min and the solvent was removed in vacuo. The product was then purified by C18 chromatography (40-90% ACN/H₂O (0.1% HCO₂H)) to give compound 93 [m/z=720 (M⁺+Na)].

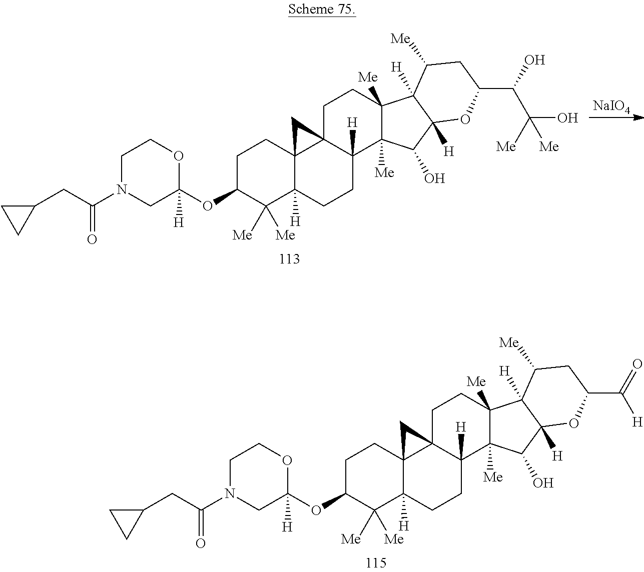

Compound 113 (260 mg) was dissolved in THF (12 mL) and H₂O (4 mL) and NaIO₄ (337 mg) was added. The solution was stirred overnight at RT and the THF was removed in vacuo. The remaining solution was diluted in CH₂Cl₂ (15 mL) and washed with H₂O (10 mL). The organic layer was then separated and removal of the solvent in vacuo gave compound 115 (233 mg).

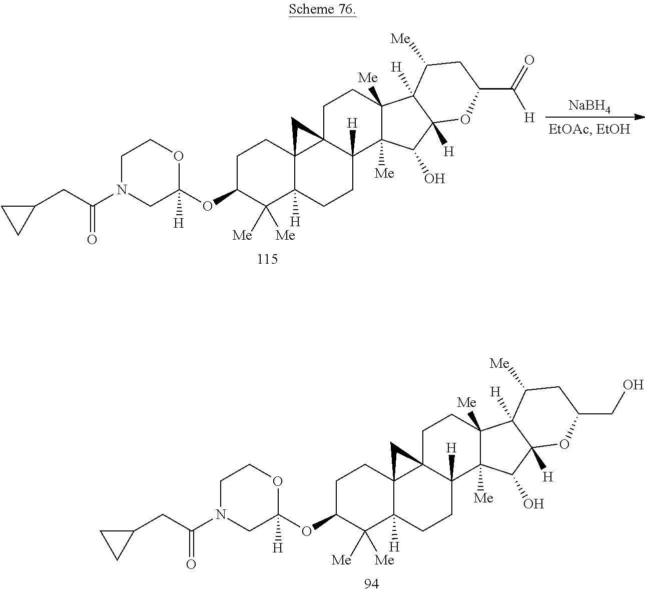

Compound 115 (23 mg) was dissolved in EtOAc (10 mL) and a solution of NaBH₄ (15 mg) in EtOH (2 mL) was added. The solution was stirred for 2 h before quenching with AcOH (100 μL) in MeOH (2 mL). The solvent was then removed in vacuo and the material was purified by silica gel chromatography (50% EtOAc/Hex to 100% EtOAc) to give compound 94 [m/z=622 (M⁺+Na)].

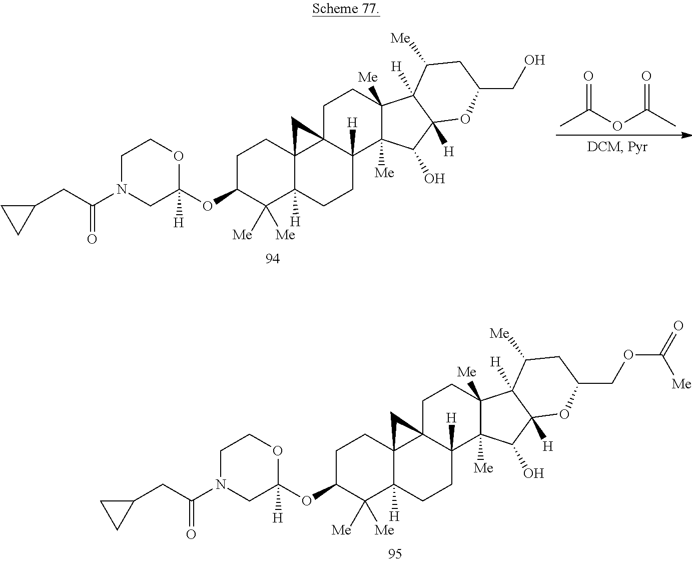

Compound 94 (13 mg) was dissolved in CH₂Cl₂ (5 mL) and pyridine (10 μL) was added. Acetic anhydride (4 μL) was added and the solution was allowed to stir for 4 h. The solution was then diluted in CH₂Cl₂ (15 mL) and washed with 1 M aqueous HCl (5 mL). The solvent was then removed in vacuo and the residue was purified by C18 column chromatography (40% ACN/H₂O to 100% ACN (0.1% HCO₂H)) to give compound 95 [m/z=664 (M⁺+Na)].

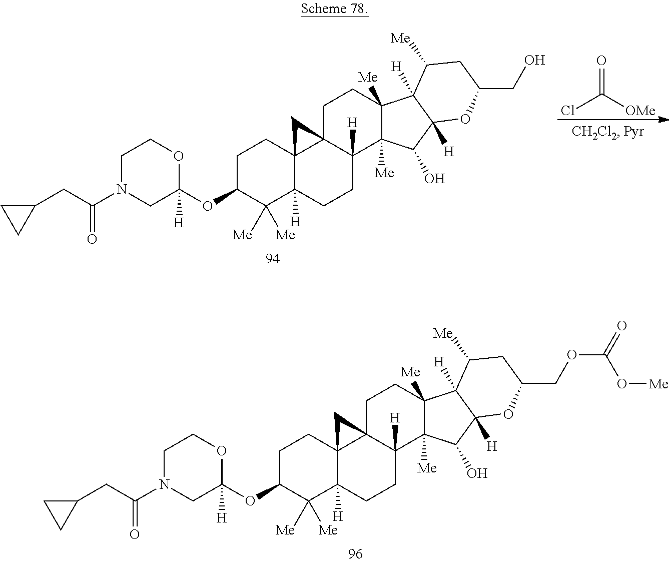

Compound 94 (13 mg) was dissolved in CH₂Cl₂ (3 mL) with DMAP (1 mg) and pyridine (30 μL). Methyl chloroformate (17 μL) was added and the solution was allowed to stir overnight. The reaction mixture was diluted in CH₂Cl₂ (15 mL) and washed with 1 M HCl (5 mL). The solvent was then removed in vacuo and the residue was purified by C18 column chromatography (40% ACN/H₂O to 100% ACN (0.1% HCO₂H)) to give compound 96 [m/z=680 (M⁺+Na)].

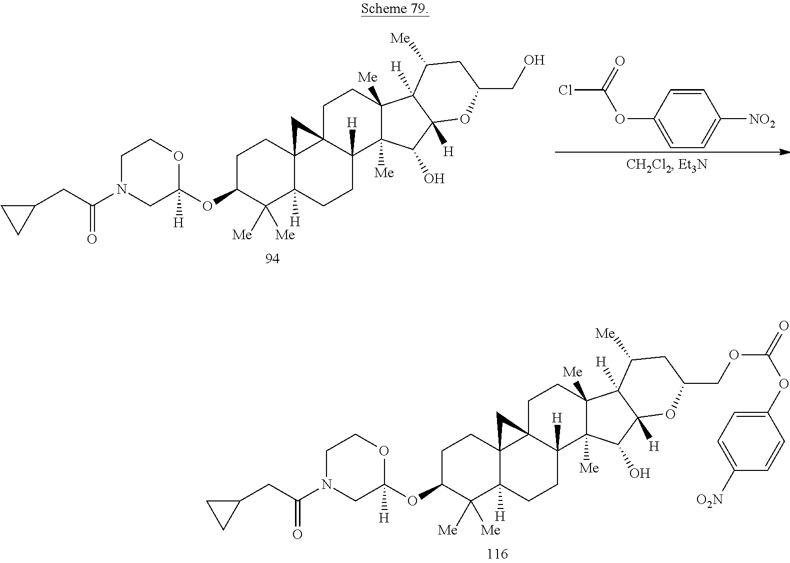

Compound 94 (22 mg) was dissolved in CH₂Cl₂ (10 mL) and DMAP (1.8 mg) and Et₃N (512 μL) was added. 4-Nitrophenyl chloroformate (28 mg) was added and the solution was allowed to stir overnight. The solution was then diluted in CH₂Cl₂ (30 mL) and washed with 1 M HCl (10 mL). The organic layer was separated and the solvent was removed in vacuo. The residue was purified by silica gel chromatography (20% EtOAc/Hex to 100% EtOAc) to give compound 116 [m/z=787 (M⁺+Na)].

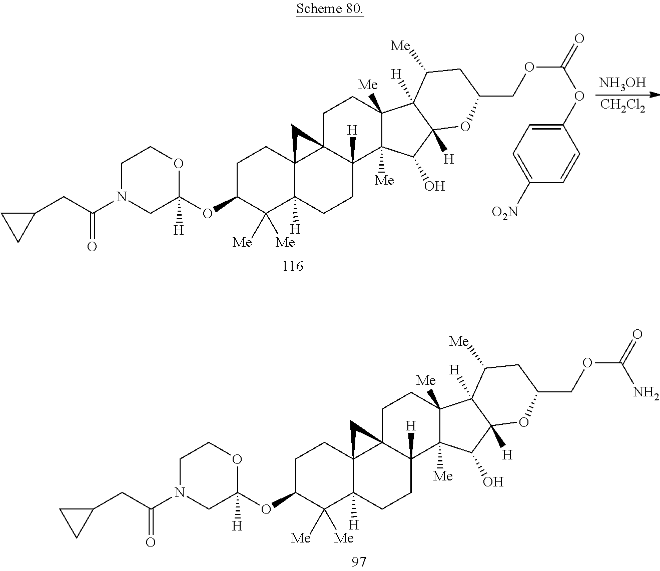

Compound 116 (11 mg) was dissolved in CH₂Cl₂ (5 mL) and 28% NH₄OH solution (500 μL) was added. The solution was stirred vigorously for 3 h. The solution was diluted in CH₂Cl₂ (15 mL) and washed twice with 10% NaHCO₃ (5 mL). The organic layer was then removed in vacuo and the residue was purified by silica gel chromatography (5-8% MeOH/CH₂Cl₂) to give compound 97 [m/z=665 (M⁺+Na)].

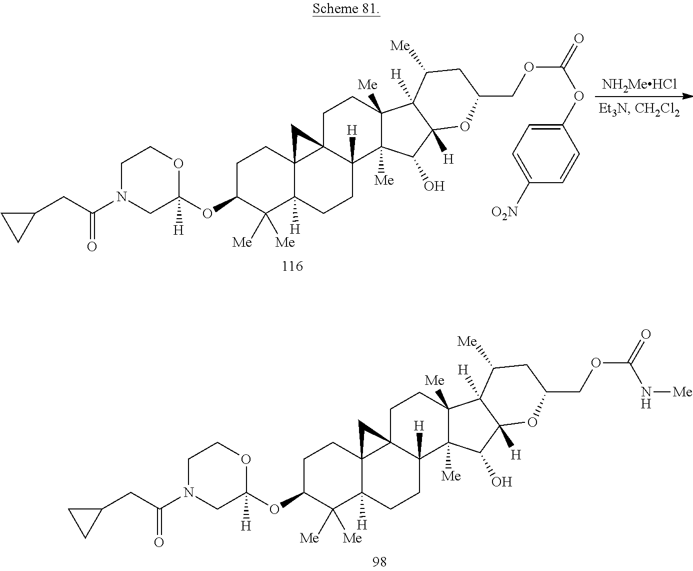

Compound 116 (11 mg) was dissolved in CH₂Cl₂ (5 mL) and a solution of methylamine hydrochloride (7 mg) and Et₃N (20 μL) in EtOH (1 mL) was added. The solution was stirred vigorously for 3 h. The solution was diluted in CH₂Cl₂ (15 mL) and washed twice with 10% NaHCO₃ (5 mL). The organic layer was then removed in vacuo and the residue was purified by silica gel chromatography (2-10% MeOH/CH₂Cl₂) to give compound 98 [m/z=679 (M⁺+Na)].

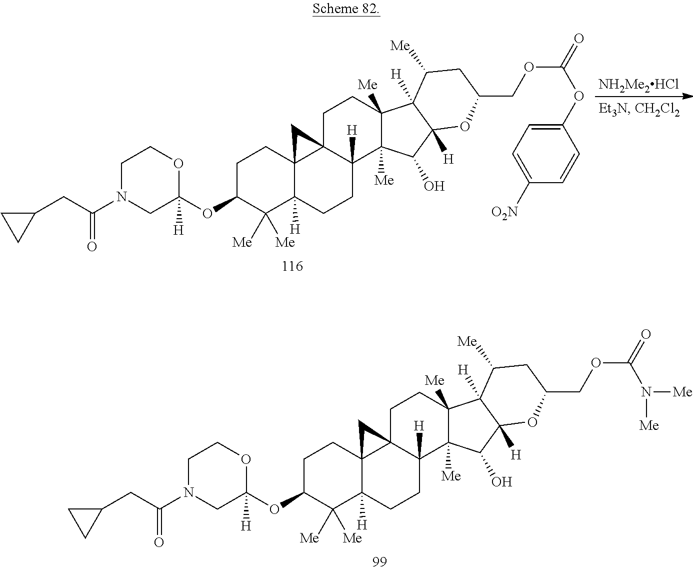

Compound 116 (11 mg) was dissolved in CH₂Cl₂ (5 mL) and a solution of dimethylamine hydrochloride (8 mg) and Et₃N (20 μL) in EtOH (1 mL) was added. The solution was stirred vigorously for 3 h. The solution was diluted in CH₂Cl₂ (15 mL) and washed twice with 10% NaHCO₃ (5 mL). The organic layer was then removed in vacuo and the residue was purified by silica gel chromatography (2-10% MeOH/CH₂Cl₂) to give compound 99 [m/z=693 (M⁺+Na)].

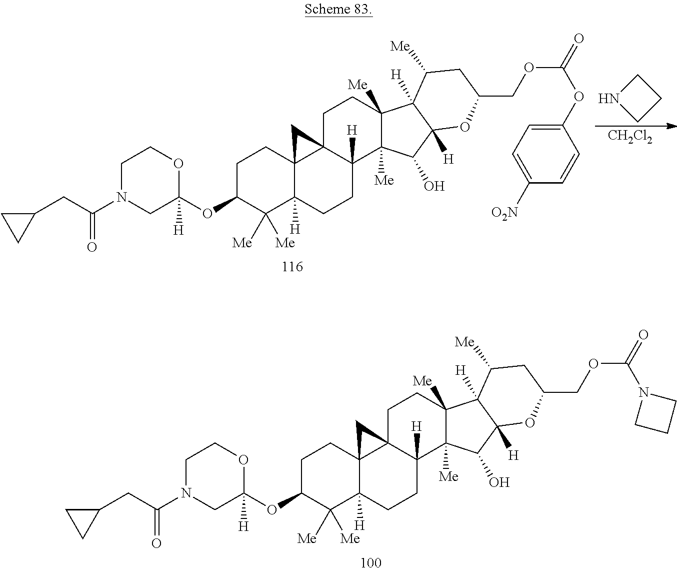

Compound 116 (11 mg) was dissolved in CH₂Cl₂ (5 mL) and azetidine (16 mg) was added. The solution was stirred vigorously for 3 h. The solution was diluted in CH₂Cl₂ (15 mL) and washed twice with 10% NaHCO₃ (5 mL). The organic layer was then removed in vacuo and the residue was purified by silica gel chromatography (2-10% MeOH/CH₂Cl₂) to give compound 100 [m/z=705 (M⁺+Na)].

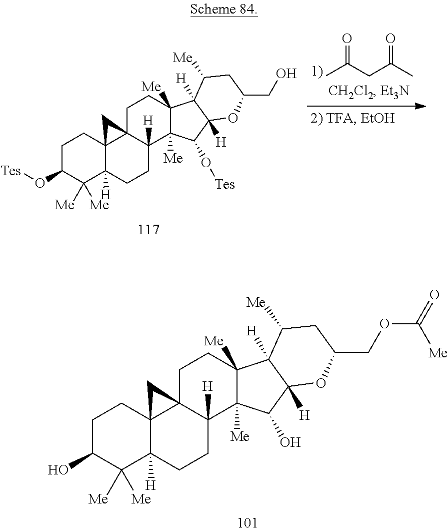

Compound 117 (23 mg) was dissolved in CH₂Cl₂ (5 mL) and DMAP and Et₃N (107 μL) was added. Acetic anhydride (33 μL) was added and the solution was stirred for 5 h. The solution was then diluted in CH₂Cl₂ (10 mL) and washed with 1 M HCl (5 ml). The solvent was removed and the residue was purified by silica gel chromatography using CH₂Cl₂. The isolated material was then dissolved in EtOH (5 mL) and TFA (10 μL) was added. The solvent was removed in vacuo and the residue was purified by silica gel chromatography (0-5% MeOH/CH₂Cl₂) to give compound 101 [m/z=497 (M⁺+Na)].

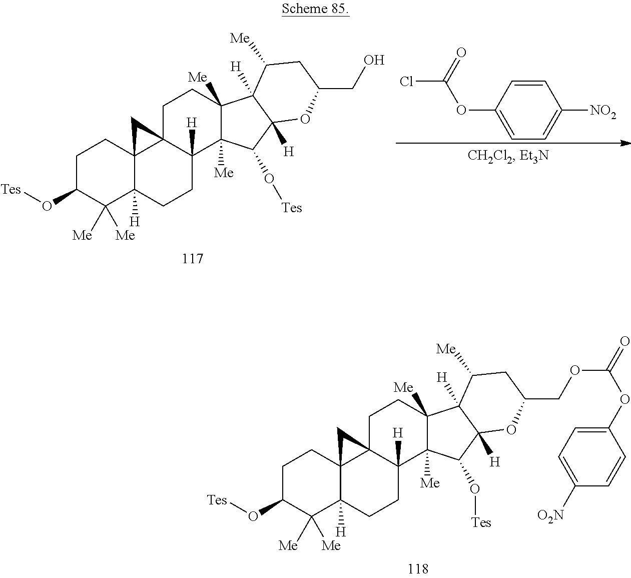

Compound 117 (60 mg) was dissolved in CH₂Cl₂ (15 mL) and DMAP (5 mg) and Et₃N (226 μL) was added. 4-Nitrophenyl chloroformate (148 mg) was then added and the solution was stirred overnight at RT. The solution was then washed with 1 M HCl (5 mL) and the organic layer was separated and the solvent removed in vacuo. The residue was purified by silica gel chromatography (0-5% MeOH/CH₂Cl₂) to give compound 118.

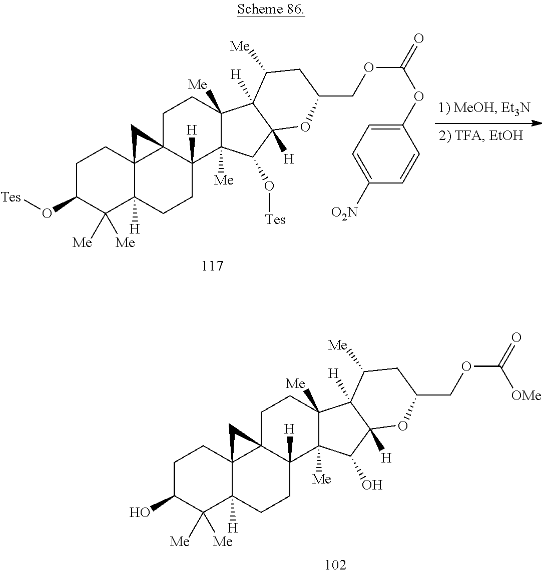

Compound 117 (20 mg) was dissolved in MeOH (15 mL) and Et₃N (107 μL) was added. The solution was allowed to stir overnight and the solvent was then removed in vacuo. The residue was dissolved in CH₂Cl₂ (15 mL) and washed with 1 M HCl (5 mL). The organic layer was separated and the solvent was removed in vacuo. The residue was dissolved in EtOH (5 ml) and TFA (10 μl) was added. The solvent was removed in vacuo and the residue was purified by silica gel chromatography (0-7% MeOH/CH₂Cl₂) to give compound 102 [m/z=513 (M⁺+Na)].

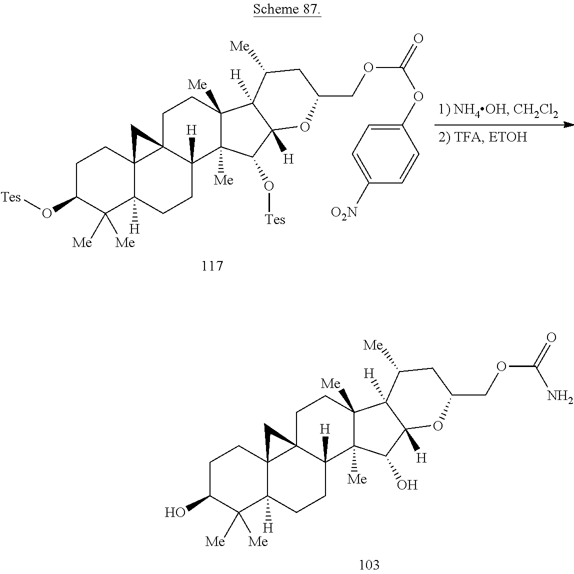

Compound 117 (20 mg) was dissolved in EtOH (1 mL) and THF (1 mL) and 28% NH₄OH (500 μL) and stirred vigorously overnight. The solution was diluted in CH₂Cl₂ (15 mL) and washed twice with NaHCO₃ (5 mL) followed by 1 M HCl (5 mL). The solvent was removed in vacuo and the residue was purified by silica gel chromatography (0-5% MeOH/CH₂Cl₂). The isolated material was then dissolved in EtOH (5 mL) and TFA (10 μL) was added. The solvent was removed in vacuo and the residue was purified by silica gel chromatography (10% EtOAc/Hex to 100% EtOAc) to give compound 103 [m/z=498 (M⁺+Na)].

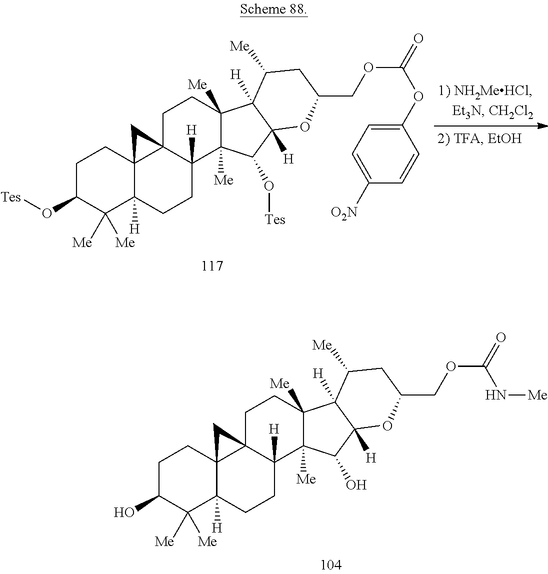

Compound 117 (11 mg) was dissolved in CH₂Cl₂ (5 mL) and a solution of methylamine hydrochloride (7 mg) and Et₃N (20 μL) in EtOH (1 mL) was added. The solution was stirred vigorously for 3 h. The solution was diluted in CH₂Cl₂ (15 mL) and washed twice with 10% NaHCO₃ (5 mL). The organic layer was then removed in vacuo and the residue was purified by silica gel chromatography (2-10% MeOH/CH₂Cl₂). The isolated material was then dissolved in EtOH (5 mL) and TFA (10 μL) was added. The solvent was removed in vacuo and the residue was purified by silica gel chromatography (10-80% EtOAc/Hex) to give compound 104 [m/z=512 (M⁺+Na)].

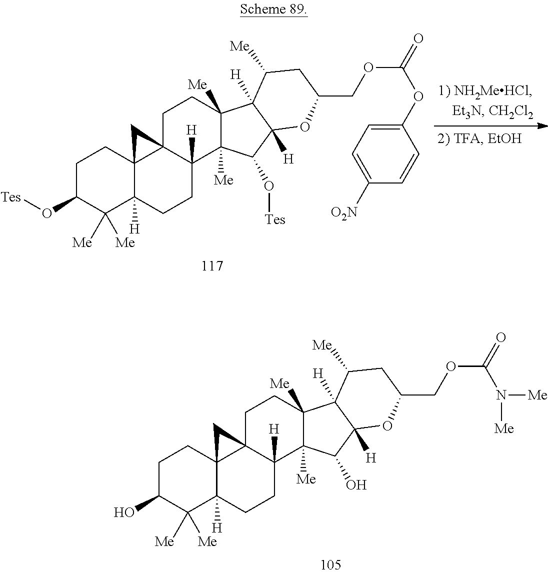

Compound 117 (20 mg) was dissolved in CH₂Cl₂ (5 mL) and a solution of dimethylamine hydrochloride (14 mg) and Et₃N (33 μL) in EtOH (1 mL) was added. The solution was stirred vigorously for 3 h. The solution was diluted in CH₂Cl₂ (15 mL) and washed twice with 10% NaHCO₃ (5 mL). The organic layer was then removed in vacuo and the residue was purified by silica gel chromatography (0-5% MeOH/CH₂Cl₂). The isolated material was then dissolved in EtOH (5 mL) and TFA (10 μL) was added. The solvent was removed in vacuo and the residue was purified by silica gel chromatography (10-80% EtOAc/Hex) to give compound 105 [m/z=526 (M⁺+Na)].

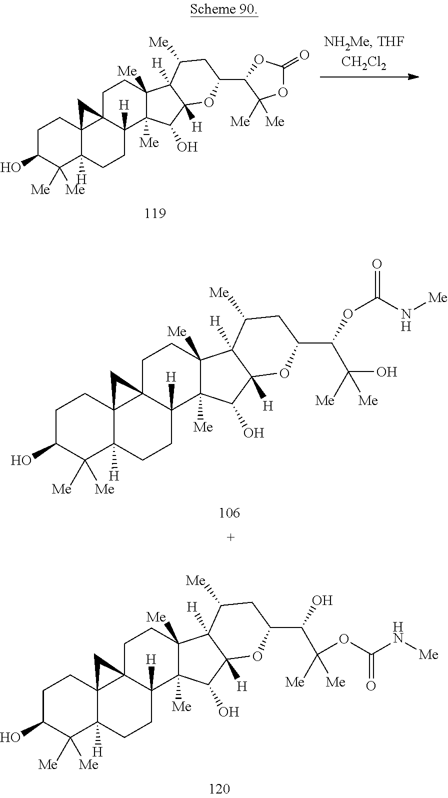

Compound 119 was dissolved in CH₂Cl₂ (15 mL) and 2M methylamine in THF (800 μL) was added and the solution was stirred at RT for 3 days. The solvent was removed in vacuo and the residue was purified by C18 column chromatography (10-60% ACN/H₂O (0.1% HCO₂H)) to separately give compounds 106 [m/z=570 (M⁺+Na)] and 120 [m/z=570 (M⁺+Na)].

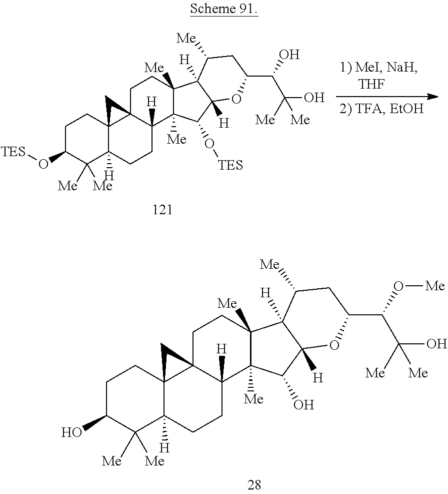

Compound 121 (40 mg) and NaH (57-63% oil dispersion, 10 mg) was dissolved in THF (3 mL) and stirred for 30 min. Iodomethane (9 μL) in THF (0.5 mL) was then added dropwise and the solution was allowed to stir overnight. The solution was then diluted in CH₂Cl₂ (30 mL) and washed with 10% aqueous NaHCO₃ and the organic layer was separated. The solvent was removed in vacuo and the residue was subjected to silica gel chromatography (0-5% MeOH in CH₂Cl₂). The alkylated product was then dissolved in EtOH (10 mL) and treated with TFA (10 μL). The solvent was then removed in vacuo and purified via silica gel chromatography (20% EtOAc/Hex to 100% EtOAc) to give compound 28 [m/z=527 (M⁺+Na)].

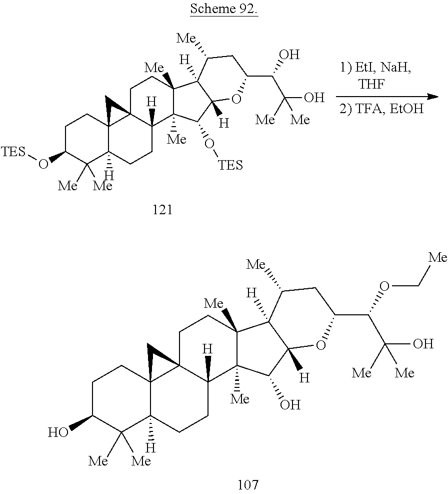

Compound 121 (40 mg) and NaH (57-63% oil dispersion, 10 mg) was dissolved in THF (3 mL) and stirred for 30 min. A solution of iodoethane (9 μL) in THF (0.5 mL) was added dropwise and the solution was stirred overnight. The solution was added to CH₂Cl₂ (15 mL) and washed with 1 M HCl. The organic layer was removed in vacuo and the residue was purified by silica gel chromatography (0-5% MeOH/CH₂Cl₂). The alkylated product was then dissolved in EtOH (10 mL) and treated with TFA (10 μL). The solvent was then removed in vacuo and purified via silica gel chromatography (20% EtOAc/Hex to 100% EtOAc) to give compound 107 [m/z=541 (M⁺+Na)].

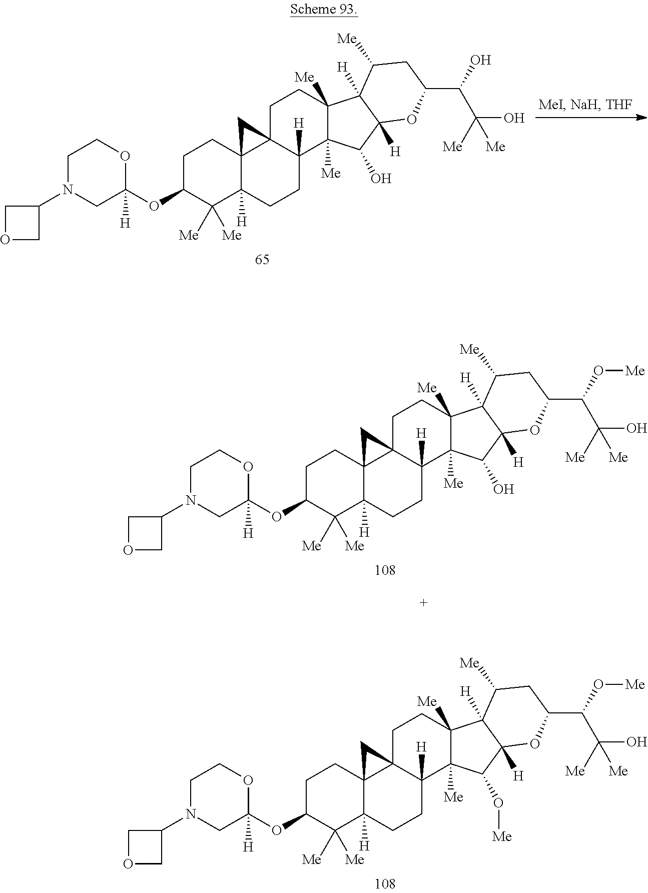

Compound 65 (20 mg) and NaH (57-63% oil dispersion, 5.3 mg) was dissolved in THF (1 mL) and stirred for 30 min. A solution of iodomethane (3 μL) in THF (1 mL) was added dropwise and the solution was stirred for 3 days. The solution was added to CH₂Cl₂ (15 mL) and washed with 1 M HCl followed by 10% NaHCO₃ until the aqueous phase remained basic. The organic layer was removed in vacuo and the residue was purified by C18 chromatography (20-55% ACN/H₂O (0.1% HCO₂H)) to separately give compounds 108 [m/z=668 (M⁺+Na)] and 109 [m/z=682 (M⁺+Na)].

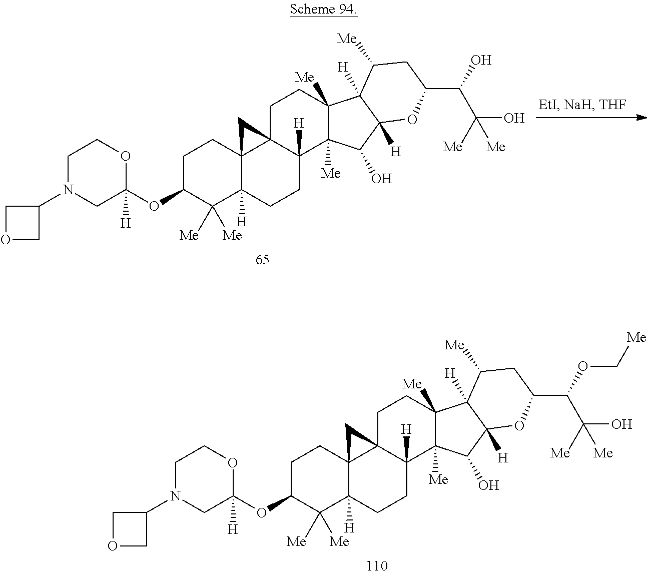

Compound 65 (19.4 mg) and NaH (57-63% oil dispersion, 18 mg) was dissolved in THF (1 mL) and stirred for 30 min. A solution of iodoethane (10 μL) in THF (1 mL) was added dropwise and the solution was stirred for 3 days. The solution was added to CH₂Cl₂ (15 mL) and washed with 1 M HCl followed by 10% NaHCO₃ until the aqueous phase remained basic. The organic layer was removed in vacuo and the residue was purified by C18 chromatography (20-55% ACN/H₂O (0.1% HCO₂H)) to give compound 110 [m/z=682 (M⁺+Na)].

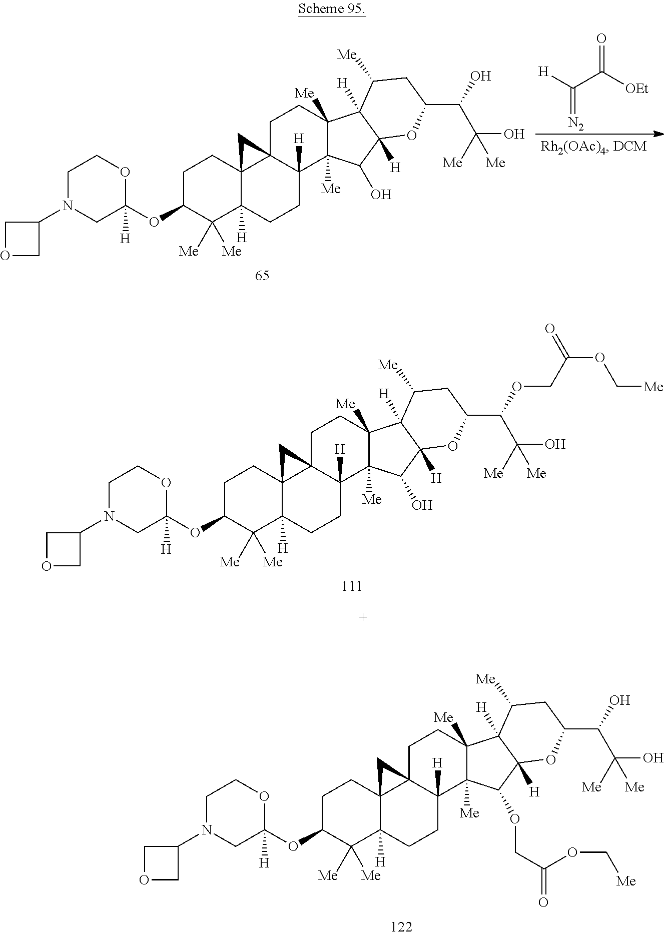

Compound 65 (20 mg) was dissolved in CH₂Cl₂ (4 mL) and ethyl diazoacetone (26 μL) in CH₂Cl₂ (1 mL) was added. To the stirred solution was added rhodium (II) acetate (10 mg) and the solution was allowed to stir for 3 h. The solution was then dissolved in CH₂Cl₂ (10 mL) and washed with H₂O (5 mL). The solvent was then removed and the residue was purified by C18 chromatography (20-60% ACN/H₂O (0.1% HCO₂H)) to separately give compounds 111 [m/z=740 (M⁺+Na)] and 122 [m/z=740 (M⁺+Na)].

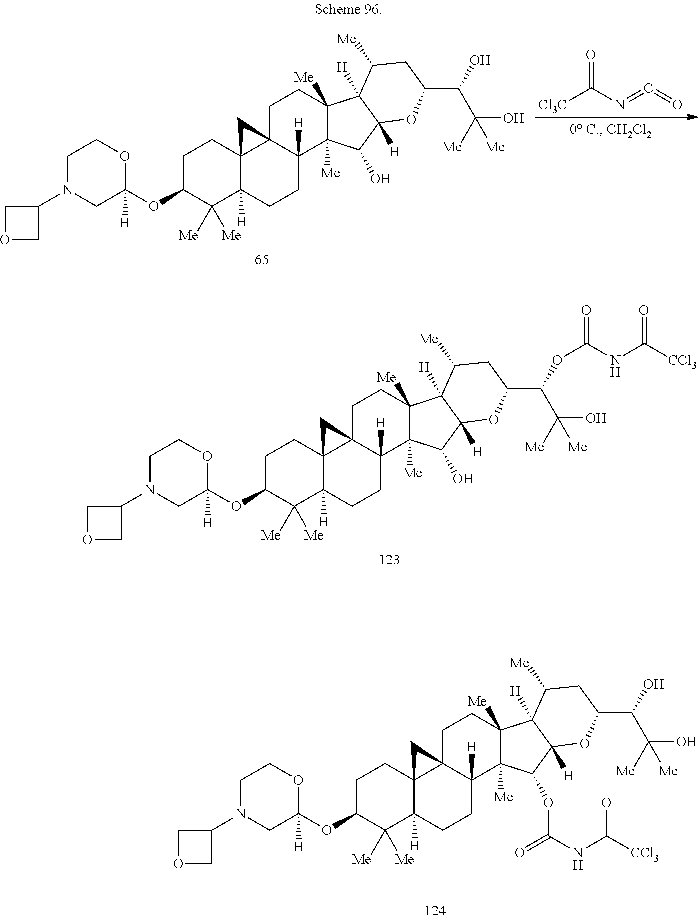

Compound 65 (20 mg) was dissolved in THF (3 mL) and cooled to 0° C. under nitrogen. Trichloroacetyl isocyanate (3.7 μL) in THF (1 mL) was added dropwise and the solution was allowed to stir at RT for 2 h. The solution was diluted in CH₂Cl₂ (15 mL) and washed with H₂O (5 mL). The organic layer was separated and the solvent was removed in vacuo. The residue was purified by C18 chromatography (20-70% ACN/H₂O (0.1% HCO₂H)) to separately give compounds 123 [m/z=843 (M⁺+Na)] and 124 [m/z=843 (M⁺+Na)].

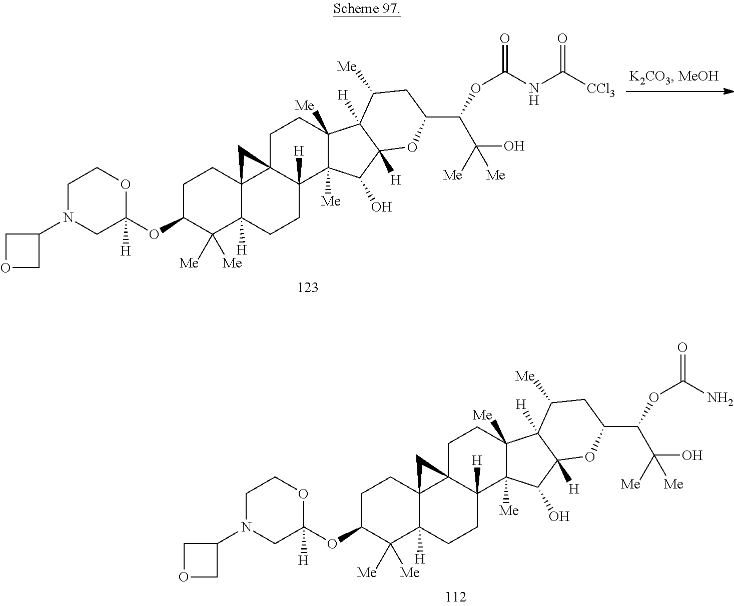

Compound 123 (6.5 mg) was dissolved in MeOH (10 mL) and K₂CO₃ (8 mg) was added. The solution was stirred overnight at RT and the solvent was removed in vacuo. The residue was dissolved in CH₂Cl₂ (15 mL) and washed with H₂O (5 mL). The organic layer was separated and the solvent removed in vacuo to give compound 112 [m/z=697 (M⁺+Na)].

### Example 10

TABLE 2
Compounds

| | |
|---|---|
| 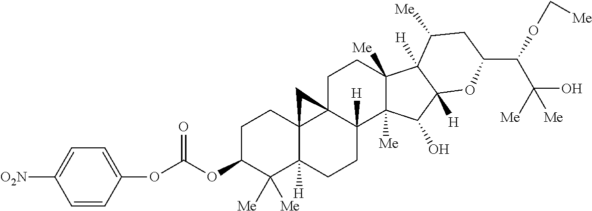 | 124 |
| 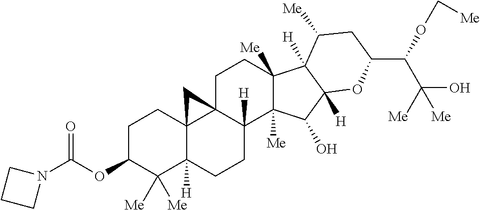 | 125 |
| 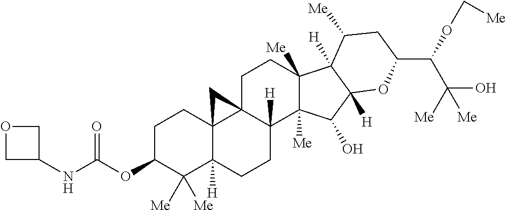 | 126 |
| 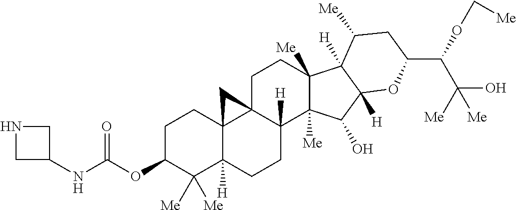 | 127 |

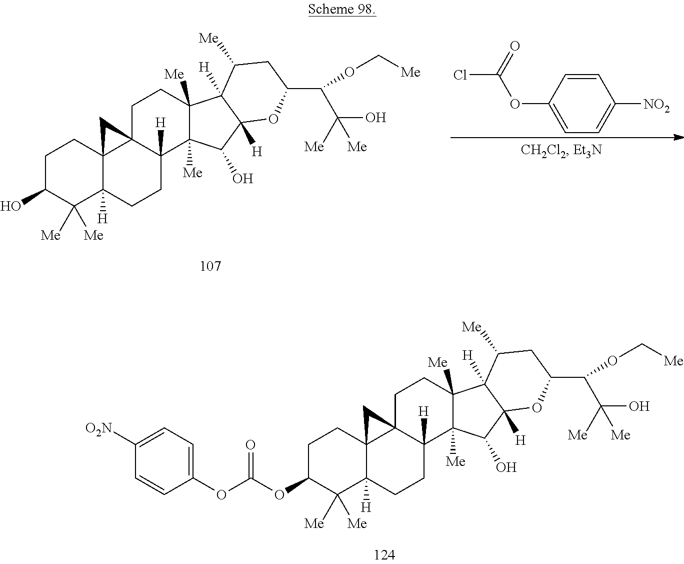

Compound 107 (46 mg) was dissolved in CH₂Cl₂ (4 mL) and DMAP (13 mg) and Et₃N (180 μL) was added. 4-Nitrophenyl chloroformate (104 mg) was then added and the solution was stirred overnight at RT. The solution was then diluted in CH₂Cl₂ (20 mL) and washed with aq. 1 M HCl (5 mL) and the organic layer was separated and the solvent removed in vacuo. The residue was purified by silica gel chromatography (20% EtOAc/Hexane to 100% EtOAc) to give compound 124 [m/z=706 (M⁺+Na)].

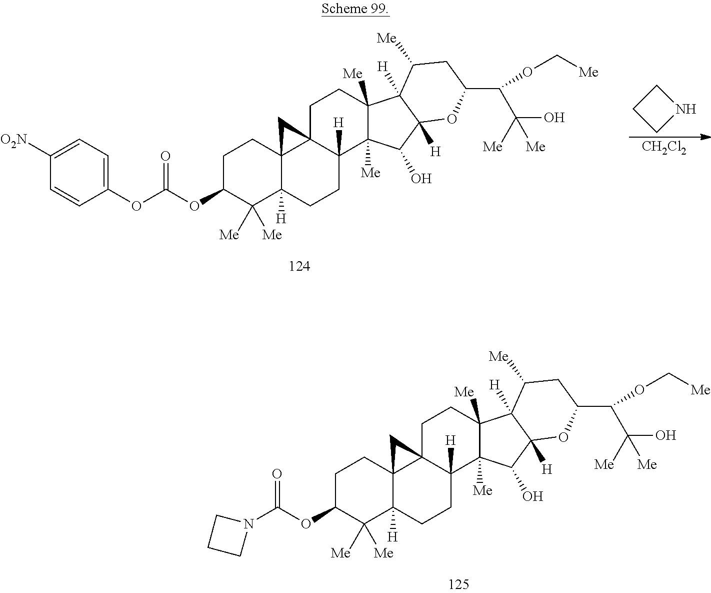

Compound 124 (10 mg) was dissolved in CH₂Cl₂ (3 mL) and azitidine (4 mg) was added. The solution was stirred vigorously for 5 h. The solution was diluted in CH₂Cl₂ (15 mL) and washed with aq. conc. HCl (5 mL) and then twice with 10% NaHCO₃ (5 mL). The organic layer was then removed in vacuo and the residue was purified by silica gel chromatography (3-10% MeOH/CH₂Cl₂) to give compound 125 [m/z=624 (M⁺+Na)].

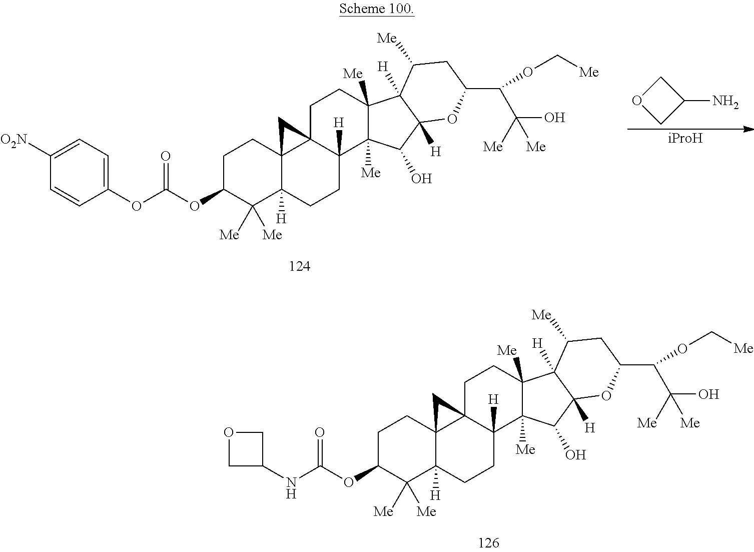

Compound 124 (10 mg) was dissolved in iPrOH (3 mL) and 3-oxetanamine (5 mg) was added. The solution was stirred vigorously for 2 days. The solvent was removed and the residue was dissolved in CH₂Cl₂ (15 mL) and washed with aq. conc. HCl (5 mL) and then twice with 10% NaHCO₃ (5 mL). The organic layer was then removed in vacuo and the residue was purified by silica gel chromatography (3-10% MeOH/CH₂Cl₂) to give compound 126 [m/z=640 (M⁺+Na)].

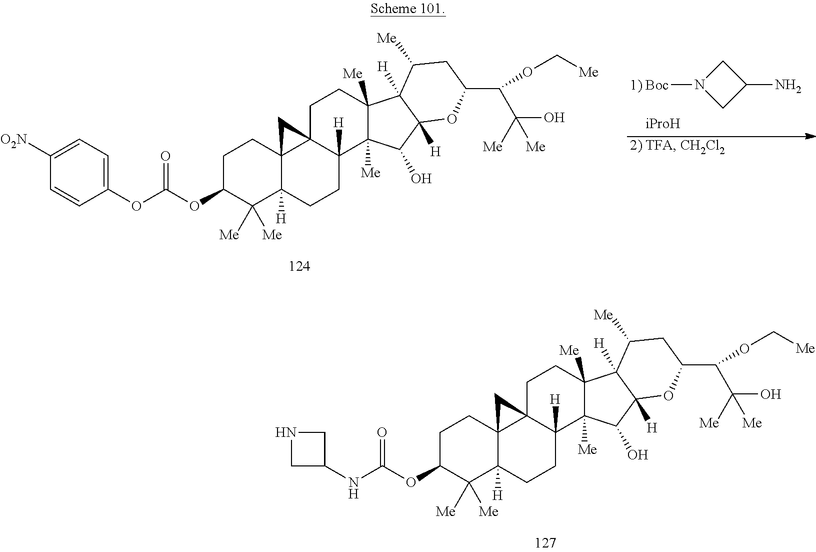

Compound 124 (10 mg) was dissolved in i-PrOH (3 mL) and 1-Boc-3-aminoazetidine (12 mg) was added. The solution was stirred vigorously for 2 days. The solvent was removed and the residue was dissolved in CH₂Cl₂ (15 mL) and washed with aq. conc. HCl (5 mL) and then twice with 10% NaHCO₃ (5 mL). The organic layer was then removed in vacuo and the residue was dissolved in CH₂Cl₂ (15 mL) and TFA (1 mL) was added. The solution was stirred at RT for 3 h and the organic layer was washed twice with NaHCO₃. The organic layer was removed in vacuo and the residue was purified by C18 chromatography (20-70% ACN/H₂O (0.1% HCO₂H)) to give compound 127 [m/z=617 (M⁺+H)].

### Example 11

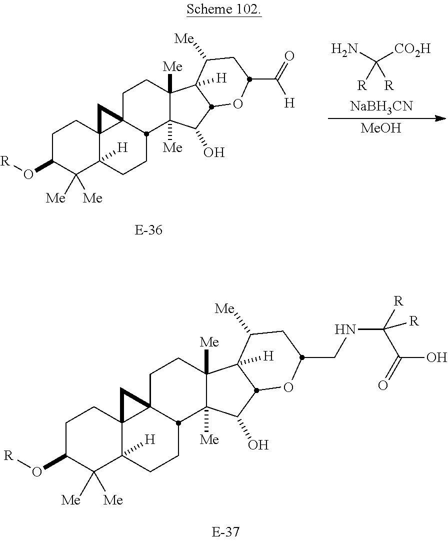

Amino Acid E-37.

A 10-mL, one-necked round-bottomed flask was charged with a solution of amino acid (100 μmol, 3 equiv) in 2 mL of MeOH and stirred at room temperature while aqueous HCl (42 μL, 2.4 M, 100 μmol, 3 equiv) was added, followed by aldehyde E-36 (33 μmol). NaBH₃CN (4.2 mg, 66 μmol, 2 equiv) was added and the resulting mixture stirred at room temperature until complete consumption of starting material was observed by LC/MS. The reaction solution was partitioned between CH₂Cl₂ (10 mL) and water (10 mL) and the aqueous phase was extracted with additional CH₂Cl₂ (2×5 mL). The combined organic phases were dried over Na₂SO₄, and purified by chromatography. Sample compounds prepared in this fashion are depicted below, along with the respective masses observed by LC/MS.

TABLE 3
Compounds

| | |
|---|---|
| 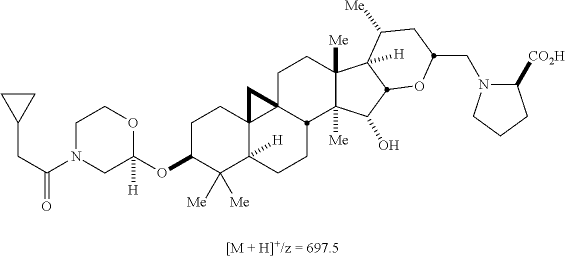 | 128 |
| 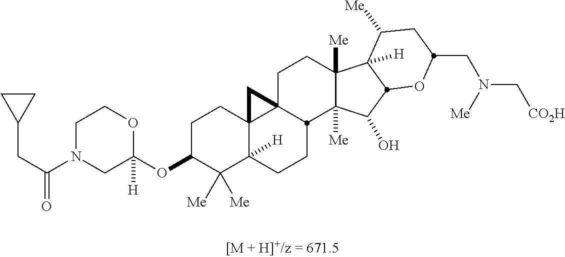 | 129 |
| 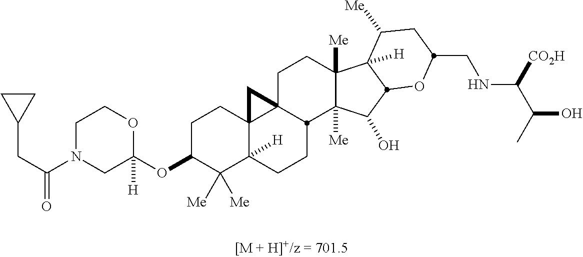 | 130 |
| 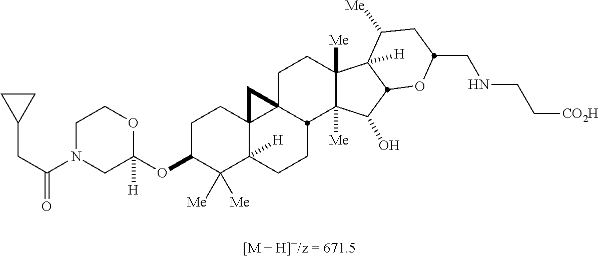 | 131 |
| 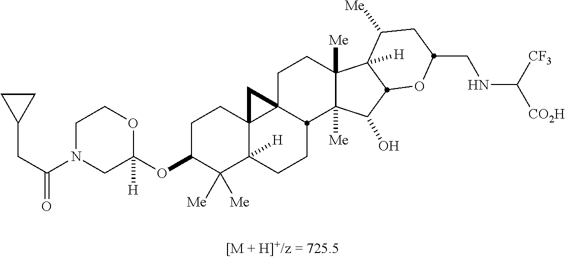 | 132 |
| 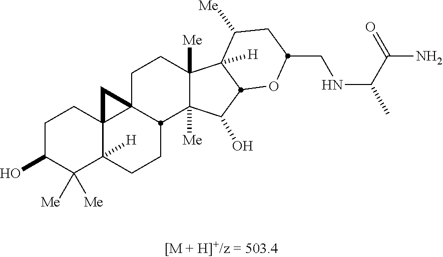 | 133 |

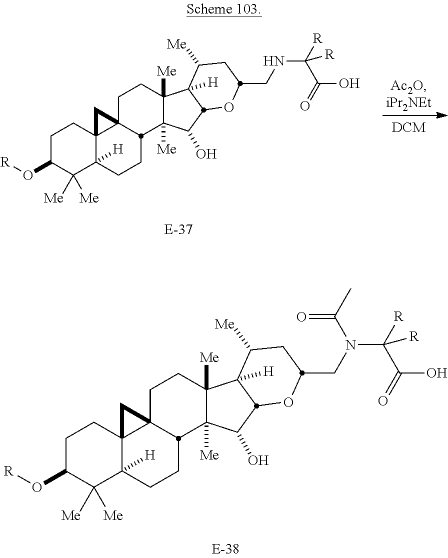

Acetamide E-38.

A 10-mL, one-necked round-bottomed flask was charged with a solution of amino acid E-37 (30 μmol, 1 equiv) in 2 mL of CH₂Cl₂ and stirred at room temperature while diisopropylethylamine (41 μL, 30.5 mg, 240 mmol, 8 equiv) was added followed by acetic anhydride (3.4 μL, 3.7 mg, 36 μmol, 1.2 equiv). The resulting mixture stirred at room temperature until complete consumption of starting material was observed by LC/MS. The reaction solution was partitioned between CH₂Cl₂ (10 mL) and water (10 mL) and the aqueous phase was extracted with additional CH₂Cl₂ (2×5 mL). The combined organic phases were dried over Na₂SO₄, and purified by chromatography. Sample compounds prepared in this fashion are depicted below, along with the respective masses observed by LC/MS.

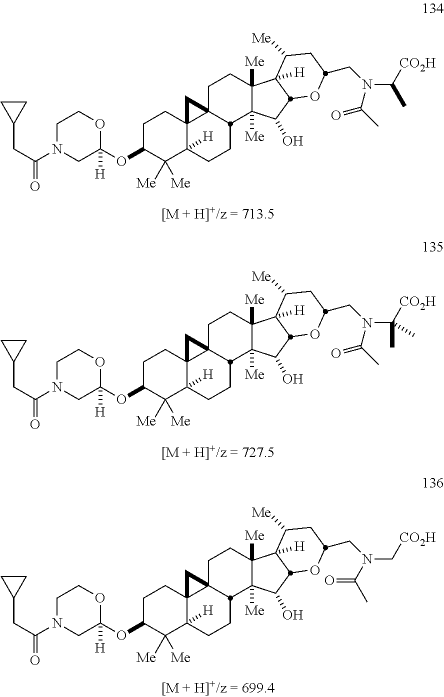

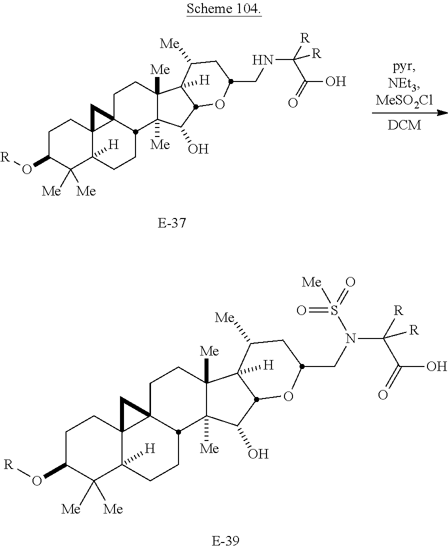

Sulfonamide E-39.

A 10-mL, one-necked round-bottomed flask was charged with a solution of amino acid E-37 (30 mmol, 1 equiv) in 2 mL of CH₂Cl₂ and stirred at room temperature while triethylamine (41 μL, 30.5 mg, 240 mmol, 8 equiv) was added followed by methanesulfonyl chloride (3.3 μL, 5.0 mg, 44 mmol, 1.4 equiv) was added. The resulting mixture stirred at room temperature for 5 h, then pyridine (10 μL, 9.8 mg, 123 mmol, 4 equiv) was added followed by methanesulfonyl chloride (5.0 μL, 7.3 mg, 66 mmol, 2.1 equiv). The resulting solution was stirred until complete consumption of starting material was observed by LC/MS. The reaction solution was partitioned between CH₂Cl₂ (10 mL) and water (10 mL) and the aqueous phase was extracted with additional CH₂Cl₂ (2×5 mL). The combined organic phases were dried over Na₂SO₄, and purified by chromatography. Sample compounds prepared in this fashion are depicted below, along with the respective masses observed by LC/MS.

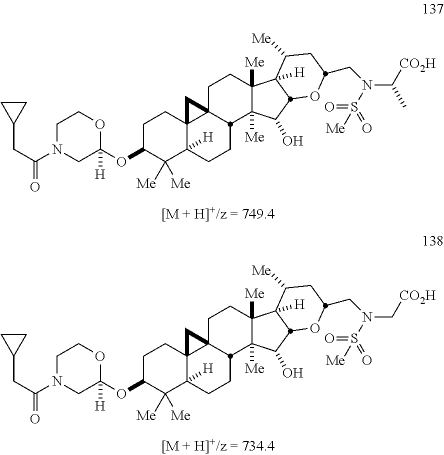

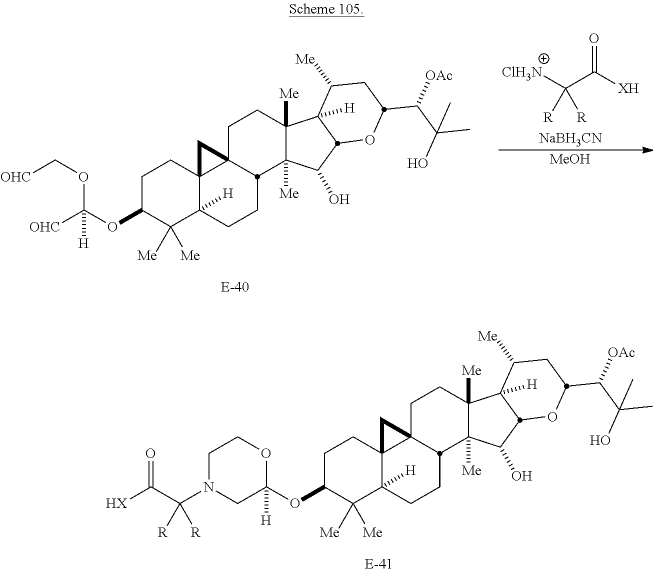

Morpholine E-41.

A 10-mL, one-necked round-bottomed flask was charged with a solution of amino acid hydrochloride salt (300 μmol, 4 equiv) and dialdehyde E-40 (49 mg, 75 iμmol) in 2 mL of MeOH and stirred at room temperature while NaBH₃CN was added in three batches (5 mg, 79 μmol, 1.0 equiv, each) spaced 45 minutes apart. The resulting mixture was stirred at room temperature for 2 additional hours then applied to a C18 reverse phase chromatography column and eluted with MeCN—H₂O containing 0.1% formic acid. Sample compounds prepared in this fashion are depicted below, along with the respective masses observed by LC/MS.

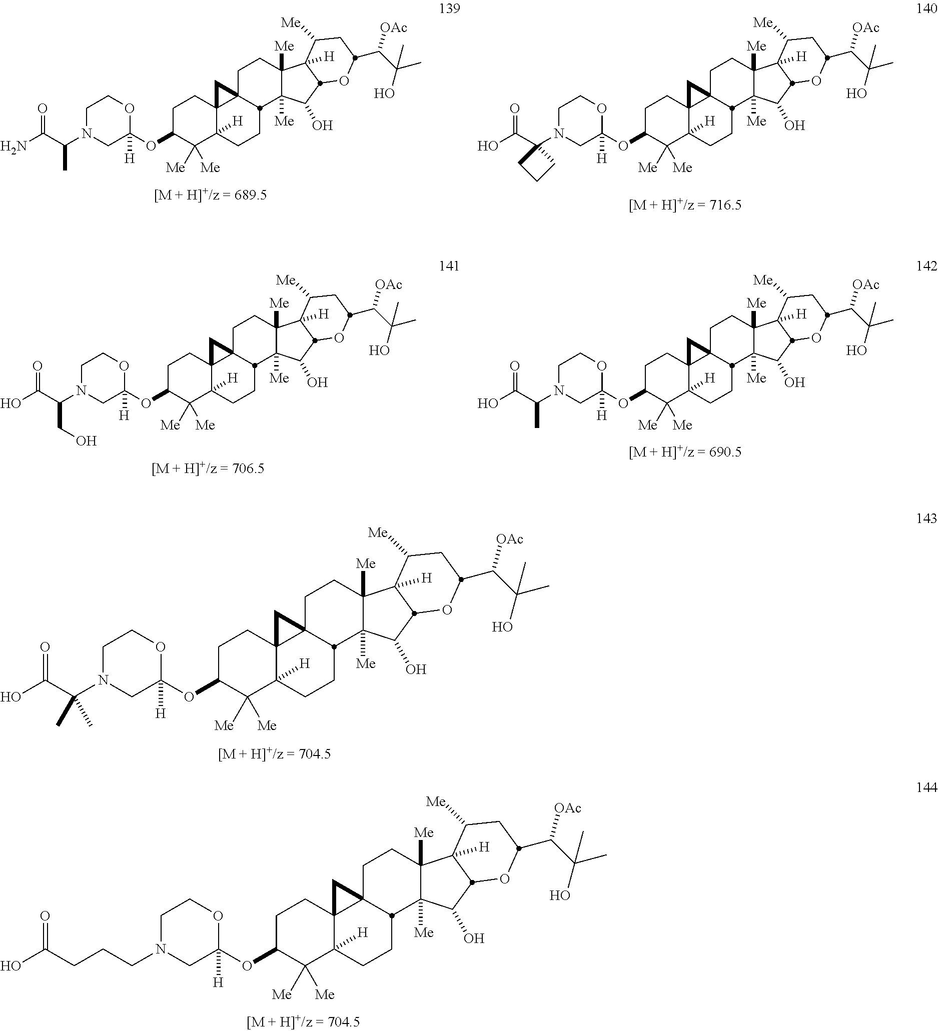

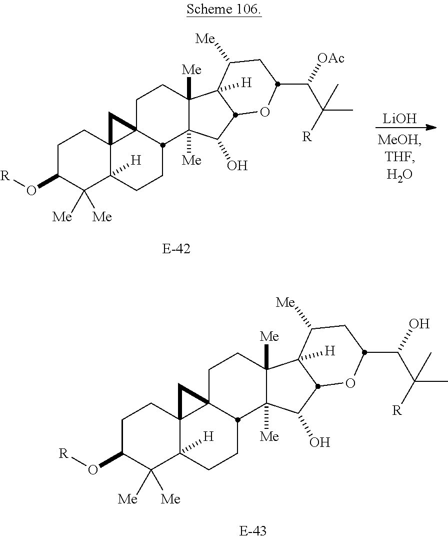

Diol E-43.

A 50-mL, one-necked, round-bottomed flask was charged with a solution of acetate E-42 (1 mmol) in THF (16 mL) and stirred at room temperature while a solution of LiOH (96 mg, 4 mmol) in water (4 mL) was added followed by THF (4 mL). The resulting mixture was stirred at room temperature until LC/MS indicated complete consumption of starting material. The reaction solution was partitioned between CH₂Cl₂ (100 mL) and saturated aqueous NaHCO₃ (100 mL) and the aqueous phase was extracted with additional CH₂Cl₂ (2×50 mL). The combined organic phases were dried over Na₂SO₄, and purified by chromatography. Sample compounds prepared in this fashion are depicted below, along with the respective masses observed by LC/MS.

TABLE 4
Compounds

| | |
|---|---|
| 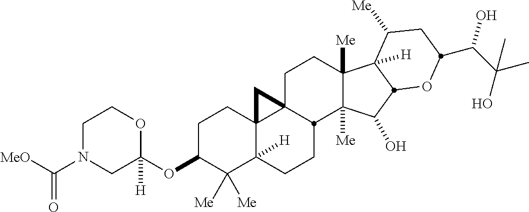 | 145 |
| 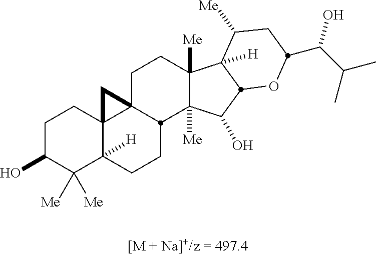 | 146 |
| 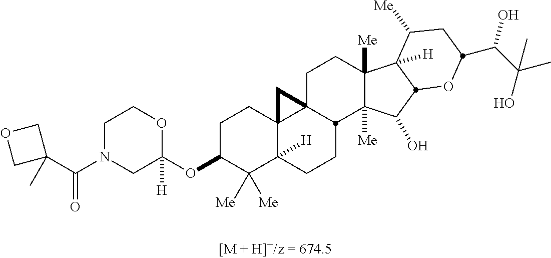 | 147 |
| 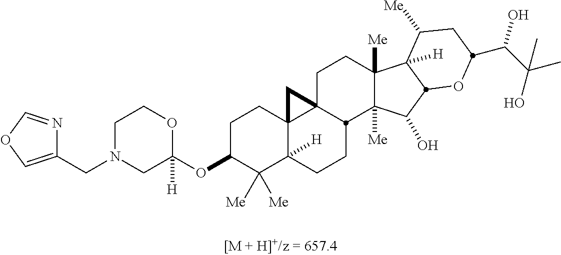 | 148 |
| 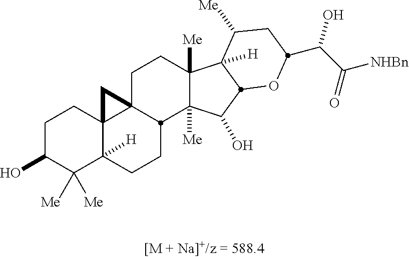 | 149 |
| 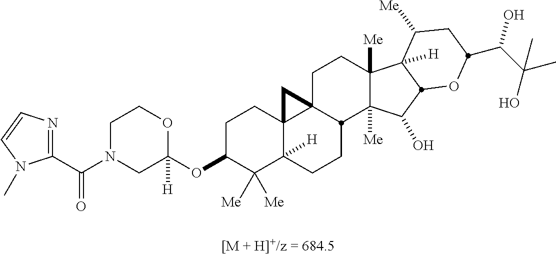 | 150 |

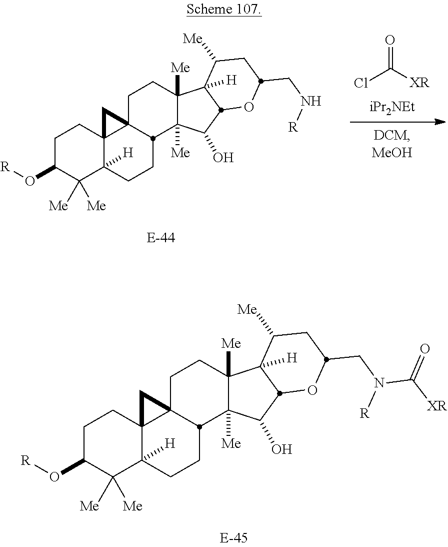

Carbonyl Compound E-45.

A 10-mL, one-necked, round-bottomed flask was charged with a solution of amine E-44 (35 μmol, 1 equiv) and diisopropylethylamine (70 μmol, 2 equiv) in DCM (2 mL) and MeOH (0.1 mL) and stirred at room temperature. An acyl chloride electrophile (38 μmol, 1.1 equiv) was added and the stirring was continued until LC/MS indicated complete consumption of starting material. The reaction solution was partitioned between CH₂Cl₂ (100 mL) and saturated aqueous NaHCO₃ (100 mL) and the aqueous phase was extracted with additional CH₂Cl₂ (2×50 mL). The combined organic phases were dried over Na₂SO₄, and purified by chromatography. Sample compounds prepared in this fashion are depicted below, along with the respective masses observed by LC/MS.

TABLE 5
Compounds

| | |
|---|---|
| 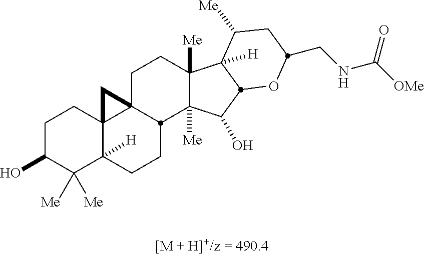 | 151 |
| 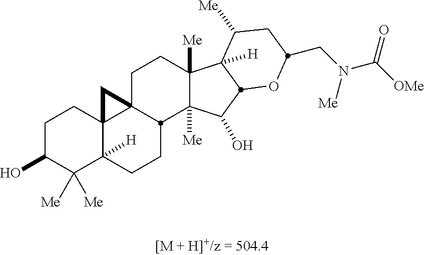 | 152 |
| 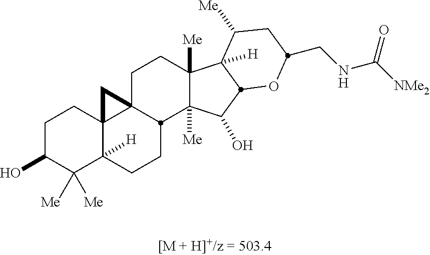 | 153 |
| 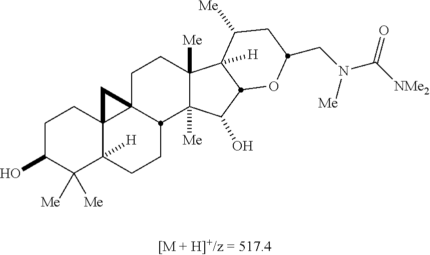 | 154 |

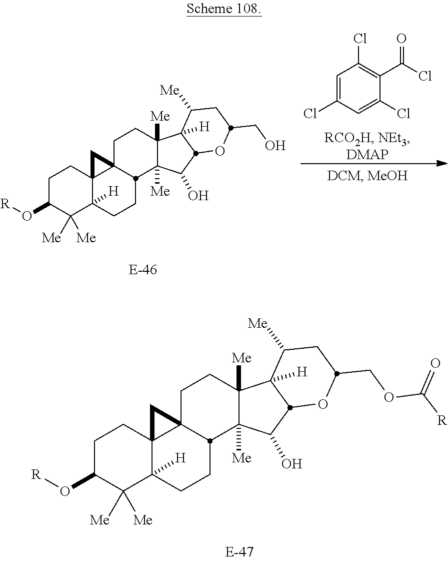

Ester E-47.

A 20-mL scinitillation vial was charged with acid (39 μmol, 1.1 equiv) and triethylamine (14.5 μL, 10.5 mg, 104 μmol, 3.0 equiv) in CH₂Cl₂ (1 mL) and stirred at rt while trichlorobenzoyl chloride (6.5 μL, 10.1 mg, 42 μmol, 1.2 equiv) was added. The resulting mixture was stirred 1 h, then alcohol E-46 (35 μmol, 1.1 equiv) was added followed by DMAP (5 mg, 41 μmol, 1.2 equiv), and stirring was continued until LC/MS indicated all starting material was consumed. The reaction solution was partitioned between CH₂Cl₂ (10 mL) and water (10 mL) and the aqueous phase was extracted with additional CH₂Cl₂ (2×5 mL). The combined organic phases were dried over Na₂SO₄, and purified by chromatography on silica gel (elution with EtOAc-hex). Sample compounds prepared in this fashion are depicted below, along with the respective masses observed by LC/MS.

TABLE 6
Compounds

| | |
|---|---|
| 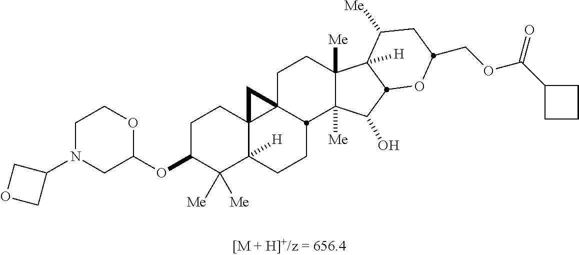 | 155 |
| 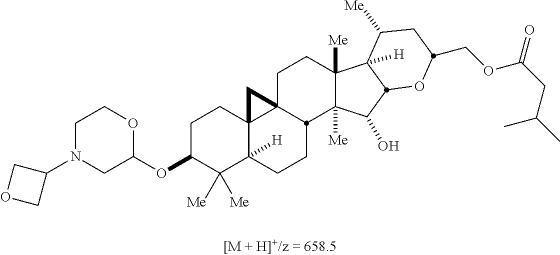 | 156 |
| 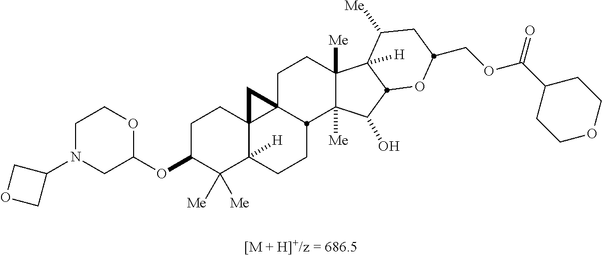 | 157 |

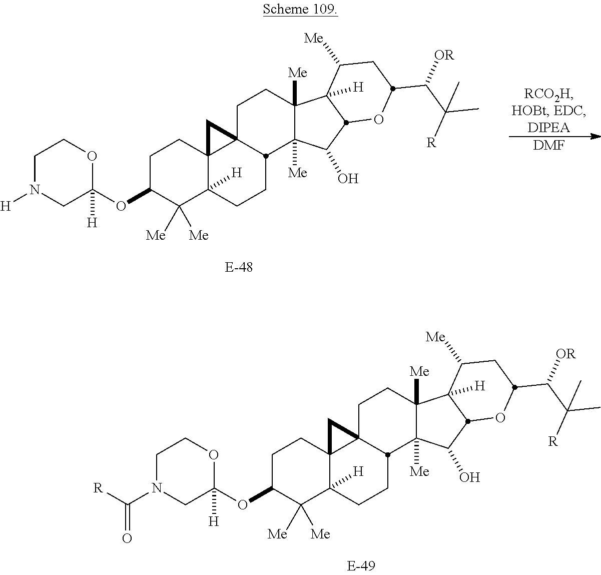

Acylmorpholine E-49.

A 20-mL scinitillation vial was charged with carboxylic acid (150 μmol, 1.5 equiv) in DMF (2 mL) and stirred at room temperature while hydroxybenzotriazole monohydrate (150 μmol, 1.5 equiv), morpholine E-48 (100 μmol, 1.0 equiv) and diispropylethylamine (500 μmol, 5 equiv) were added sequentially. 1-Ethyl-3-(3-dimethylaminopropyl)carbodiimide hydrochloride (200 μmol, 2 equiv) was added and the resulting mixture was stirred at room temperature until LC/MS indicated complete consumption of starting material then applied to a C18 reverse phase chromatography column and eluted with MeCN—H₂O containing 0.1% formic acid. Sample compounds prepared in this fashion are depicted below, along with the respective masses observed by LC/MS.

TABLE 7
Compounds

| | |
|---|---|
| 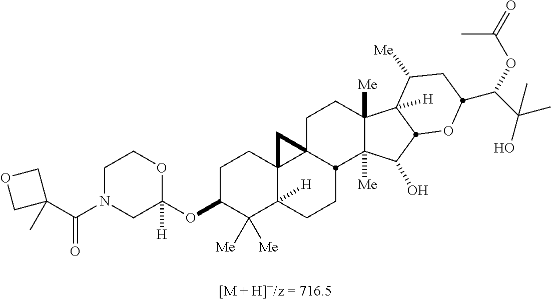 | 158 |
| 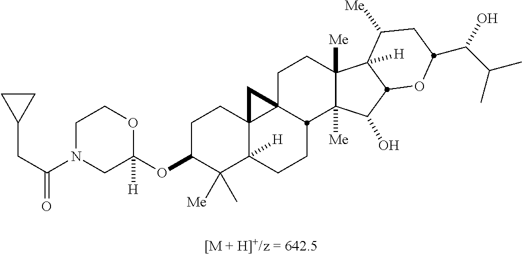 | 159 |
| 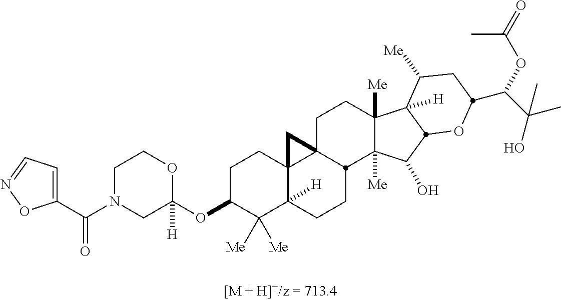 | 160 |
| 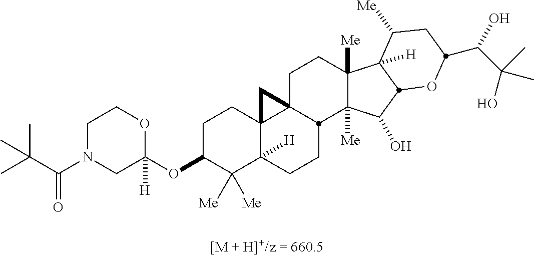 | 161 |
| 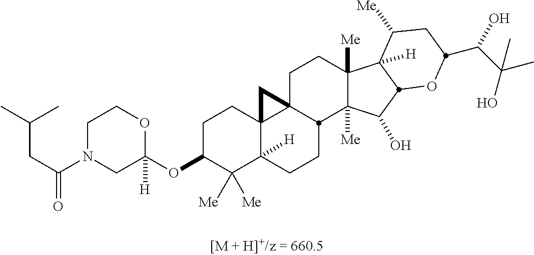 | 162 |

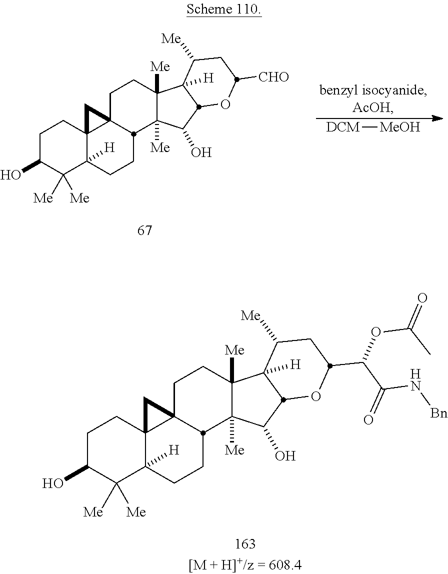

Amide 163.

A 2-mL vial was charged with aldehyde 67 (25 mg, 58 μmol, 1.0 equiv) in a solution of DCM (0.18 mL) and MeOH (0.02 mL). Benzyl isocyanide (19.2 mg, 20 μL, 164 μmol, 2.8 equiv) and AcOH (6.3 mg, 6 μL, 105 μmol, 1.8 equiv) were added and the resulting mixture was stirred overnight at room temperature and concentrated. The crude product was purified by chromatography on silica gel (elution with EtOAc-hexanes) to provide 29 mg of the desired product.

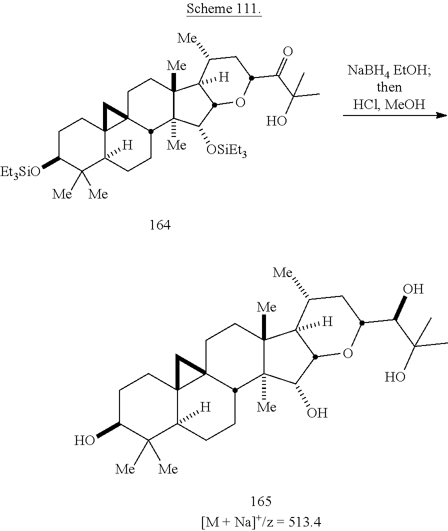

Tetraol 165.

A 10-mL flask was charged with a solution of ketone 164 (43 mg, 60 μmol, 1.0 equiv) in EtOH (2 mL) and stirred at room temperature while NaBH₄ (3 mg, 79 μmol, 1.3 equiv) was added and stirring was continued for 2 h. The reaction solution was partitioned between CH₂Cl₂ (20 mL) and 5% aqueous citric acid (20 mL) and the aqueous phase was extracted with additional CH₂Cl₂ (2×10 mL). The crude product was dissolved in MeOH (2 mL) and 2.4 M HCl (100 μL) was added. The reaction mixture was stirred for 1 h, then partitioned between CH₂Cl₂ (20 mL) and saturated aqueous NaHCO₃ (20 mL) and the aqueous phase was extracted with additional CH₂Cl₂ (2×10 mL). The crude product was purified by chromatography on silica gel (elution with DCM-MeOH) to provide 11 mg of the desired product.

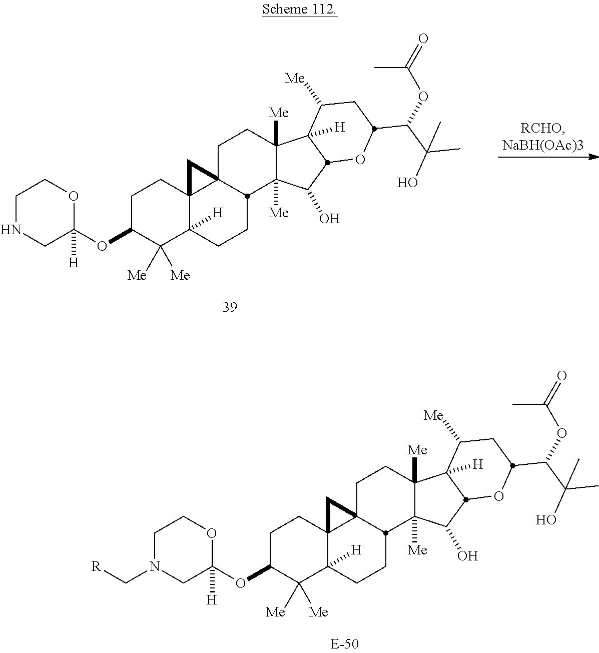

Morpholine E-50.

A 10-mL flask was charged with a solution of morpholine 39 (100 μmol, 1.0 equiv) and aldehyde (140 μmol, 1.4 equiv) in EtOH (0.9 mL), AcOH (0.1 mL), and DCM (0.1 mL) and stirred at room temperature while NaBH(OAc)₃ (120 μmol, 1.2 equiv) was added. The reaction was stirred at room temperature until LC/MS indicated complete consumption of starting material, and was then applied to a C18 reverse phase chromatography column and eluted with MeCN—H₂O containing 0.1% formic acid. Sample compounds prepared in this fashion are depicted below, along with the respective masses observed by LC/MS.

TABLE 8
Compounds

| | |
|---|---|
| 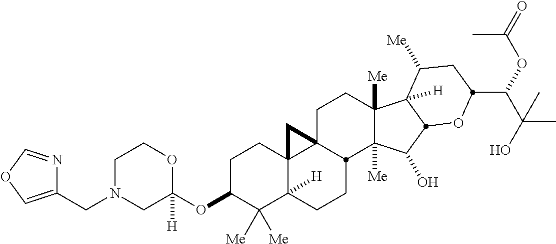 | 166 |
| [M + H]⁺/z = 699.5 | |
| 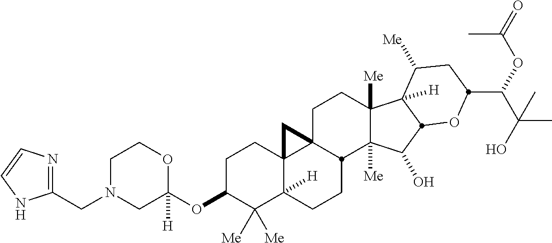 | 167 |
| [M + H]⁺/z = 698.5 | |
| 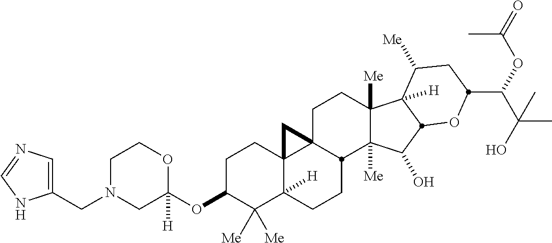 | 168 |
| [M + H]⁺/z = 698.5 | |

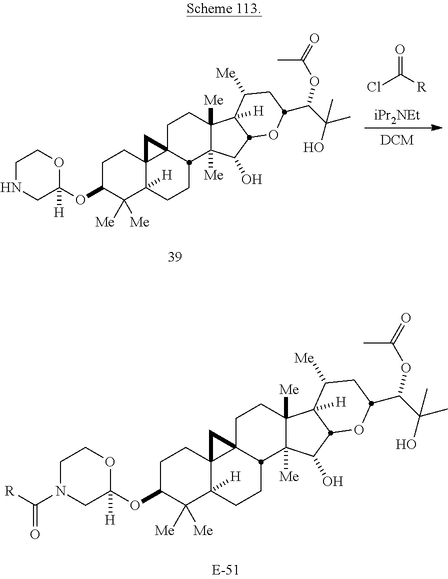

Acylmorpholine E-51.

A 10-mL flask was charged with morpholine 39 (100 iμmol, 1.0 equiv) in DCM (1 mL) and stirred at room temperature while diisopropylethylamine (63.5 mg, 87 μL, 500 μmol, 5.0 equiv) was added followed by an acyl chloride (120 μmol, 1.2 equiv). Stirring was continued until LC/MS indicated all starting material was consumed. The reaction solution was partitioned between CH₂Cl₂ (10 mL) and water (10 mL) and the aqueous phase was extracted with additional CH₂Cl₂ (2×5 mL). The combined organic phases were dried over Na₂SO₄, and purified by chromatography on silica gel (elution with EtOAc-hex). Sample compounds prepared in this fashion are depicted below, along with the respective masses observed by LC/MS.

TABLE 9
Compounds

| | |
|---|---|
| 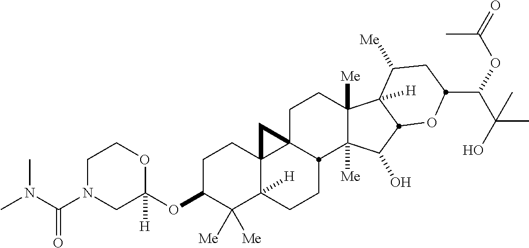 | 169 |
| [M + H]⁺/z = 689.5 | |
| 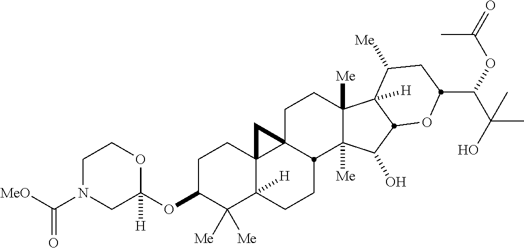 | 170 |
| [M + H]⁺/z = 676.4 | |
| 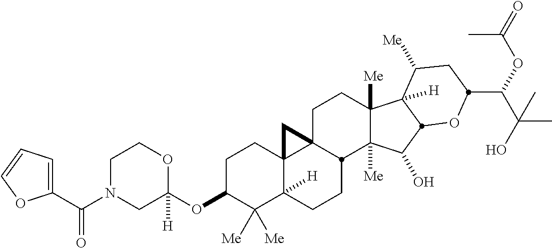 | 171 |
| [M + H]⁺/z = 711.4 | |

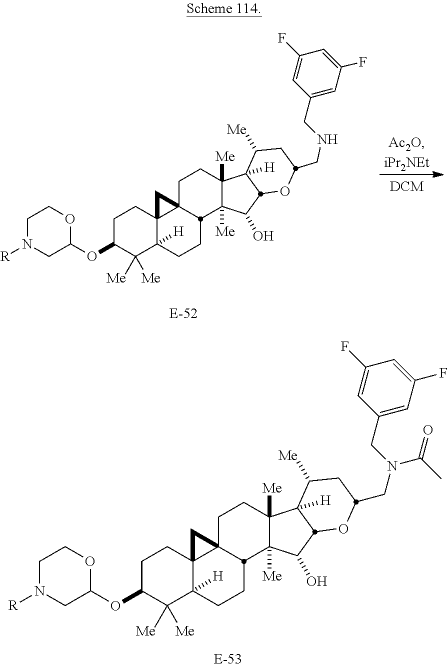

Acetamide E-53.

A 10-mL, one-necked round-bottomed flask was charged with a solution of amino acid E-52 (100 μmol, 1 equiv) in 2 mL of CH₂Cl₂ and stirred at room temperature while triethylamine (70 L, 50.5 mg, 500 μmol, 5 equiv) was added followed by acetic anhydride (11.3 μL, 12.2 mg, 120 μmol, 1.2 equiv) was added. The resulting mixture was stirred at room temperature until complete consumption of starting material was observed by LC/MS. The reaction solution was partitioned between CH₂Cl₂ (10 mL) and water (10 mL) and the aqueous phase was extracted with additional CH₂Cl₂ (2×5 mL). The combined organic phases were dried over Na₂SO₄, and purified by chromatography (elution with EtOAc-hexanes). Sample compounds prepared in this fashion are depicted below, along with the respective masses observed by LC/MS.

TABLE 10
Compounds

| | |
|---|---|
| 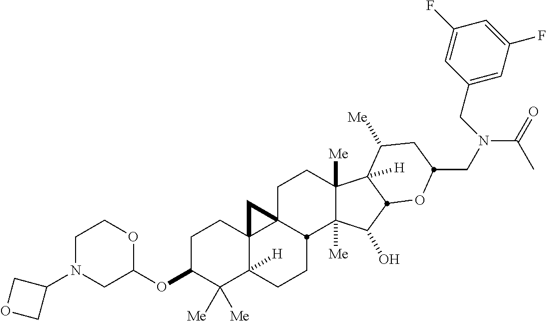 | 172 |
| [M + H]⁺/z = 712.5 | |
| 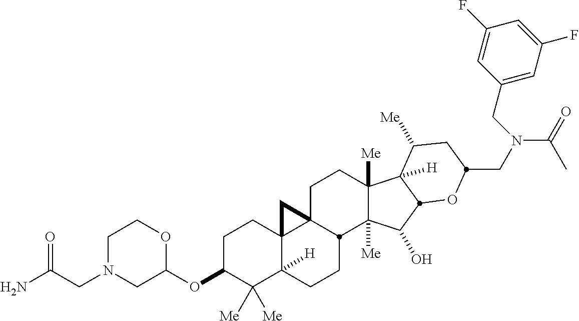 | 173 |
| [M + H]⁺/z = 742.5 | |

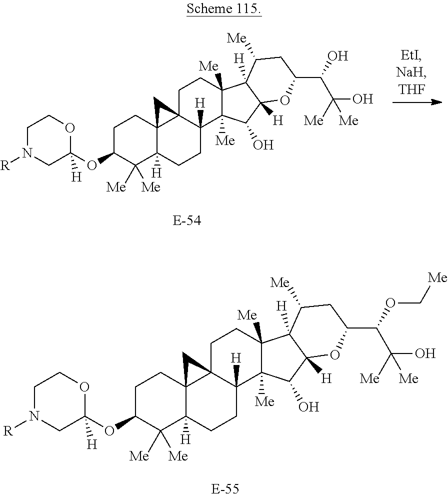

For synthetic procedures see Example 9 (Methods of making compounds of Table 1)

TABLE 11
Compounds

| | |
|---|---|
| 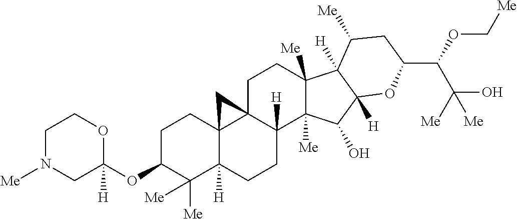 | 174 |
| [M + H]⁺/z = 618.5 | |
| 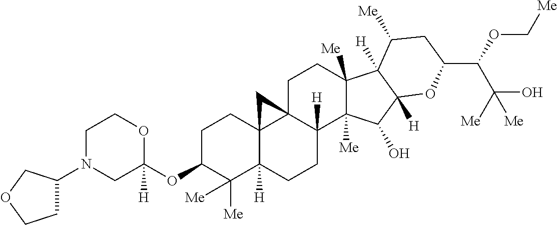 | 175 |
| [M + H]⁺/z = 674.5 | |
| 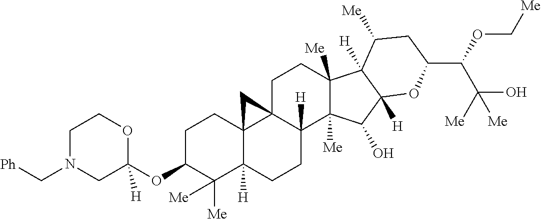 | 176 |
| [M + H]⁺/z = 694.5 | |
| 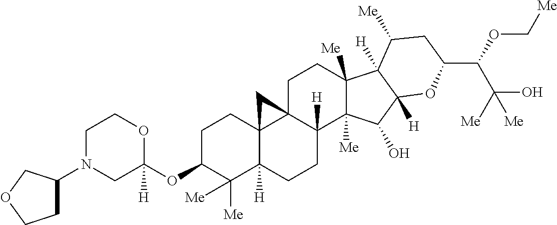 | 177 |
| [M + H]⁺/z = 674.5 | |
| 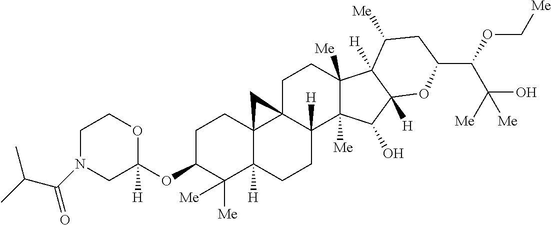 | 178 |
| [M + H]⁺/z = 674.5 | |

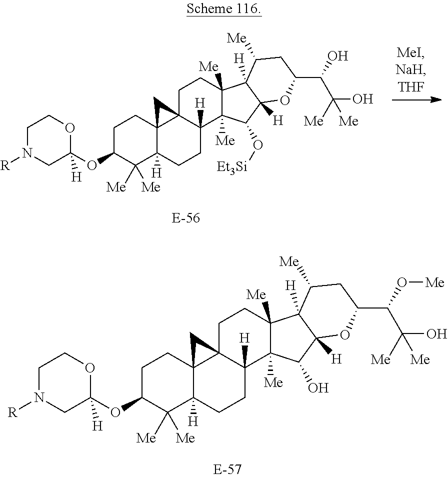

For synthetic procedures see Example 9 (Methods of making compounds of Table 1)

TABLE 12
Compounds

| | |
|---|---|
| 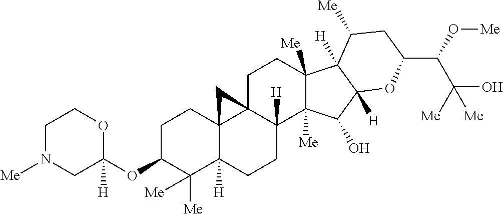 | 179 |
| [M + H]⁺/z = 604.5 | |
| 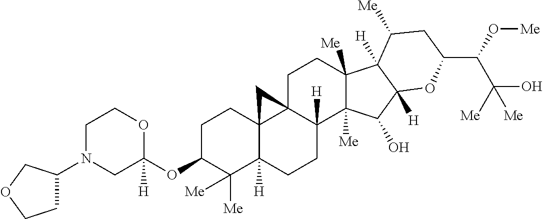 | 180 |
| [M + H]⁺/z = 660.5 | |
| 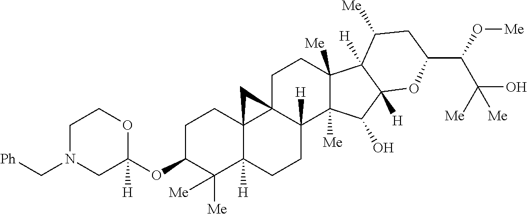 | 181 |
| [M + H]⁺/z = 680.5 | |
| 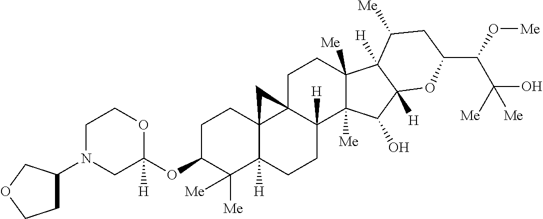 | 182 |
| [M + H]⁺/z = 660.5 | |
| 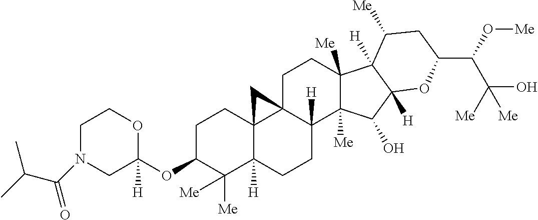 | 183 |
| [M + H]⁺/z = 660.5 | |

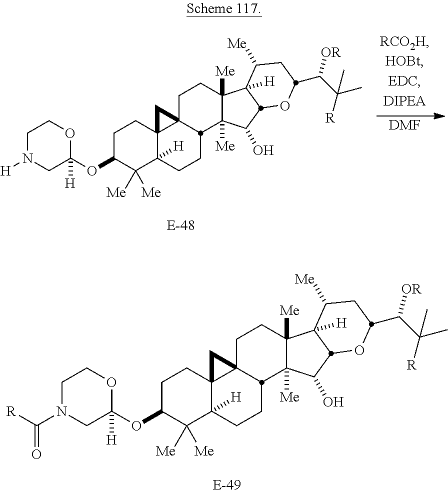

Acylmorpholine E-48.

A 20-mL scinitillation vial was charged with carboxylic acid (150 μmol, 1.5 equiv) in DMF (2 mL) and stirred at room temperature while hydroxybenzotriazole monohydrate (150 μmol, 1.5 equiv), morpholine E-48 (100 μmol, 1.0 equiv) and diispropylethylamine (500 μmol, 5 equiv) were added sequentially. 1-Ethyl-3-(3-dimethylaminopropyl)carbodiimide hydrochloride (200 μmol, 2 equiv) was added and the resulting mixture was stirred at room temperature until LC/MS indicated complete consumption of starting material then applied to a C18 reverse phase chromatography column and eluted with MeCN—H₂O containing 0.1% formic acid. Sample compounds prepared in this fashion are depicted below, along with the respective masses observed by LC/MS.

TABLE 13
Compounds

| | |
|---|---|
| 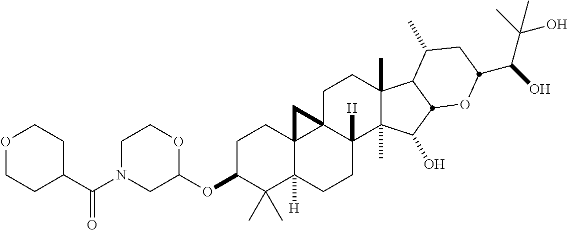 | 184 |
| 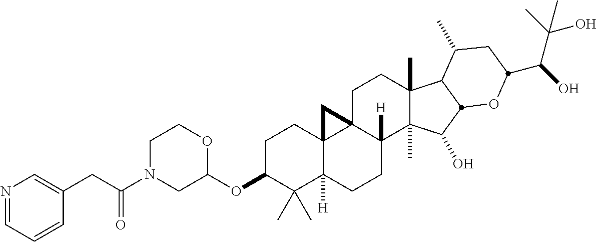 | 185 |
| 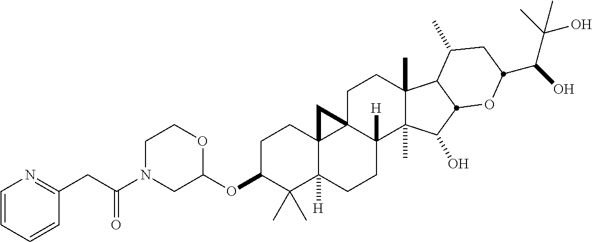 | 186 |
| 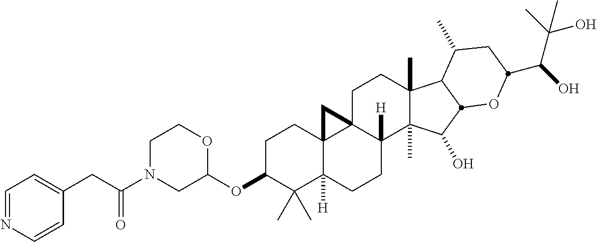 | 187 |
| 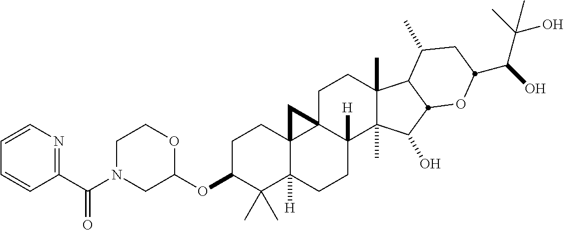 | 188 |
| 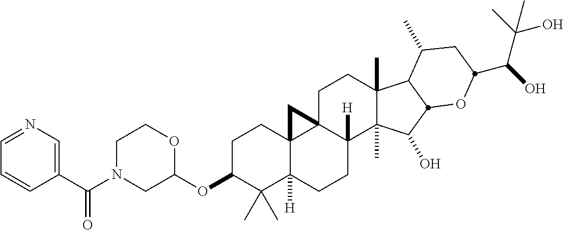 | 189 |
| 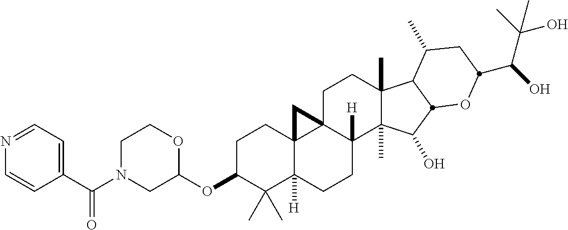 | 190 |
| 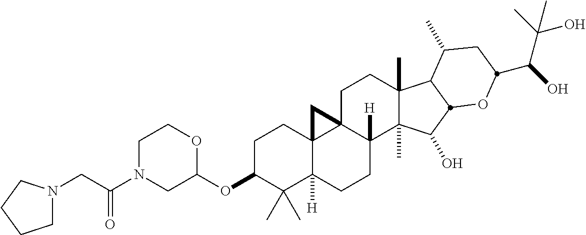 | 191 |
| 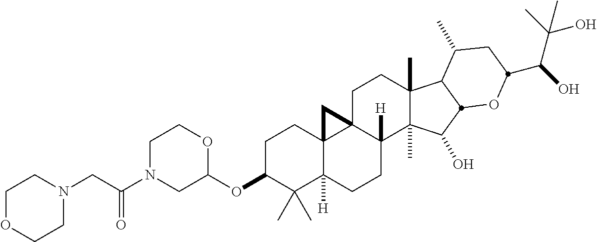 | 192 |
| 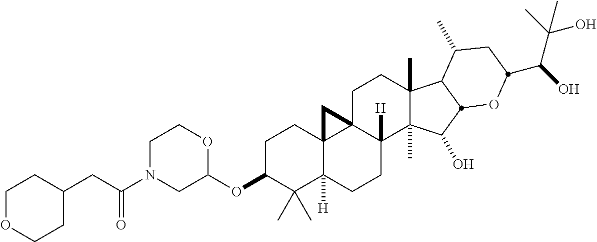 | 193 |
| 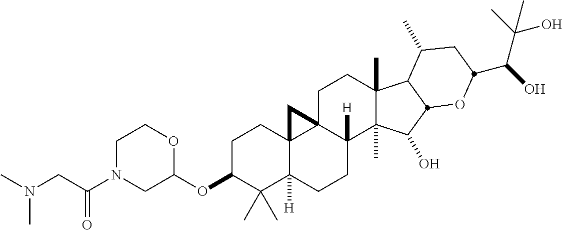 | 194 |
| 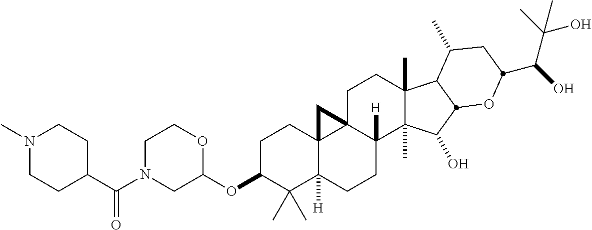 | 195 |
| 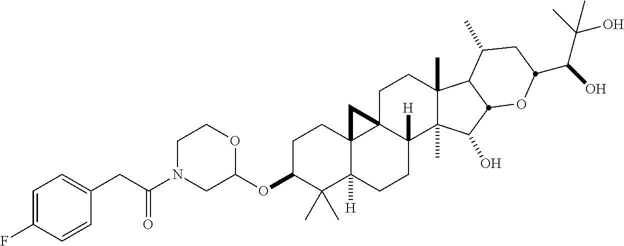 | 196 |
| 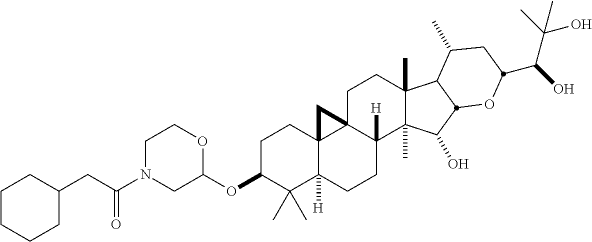 | 197 |
| 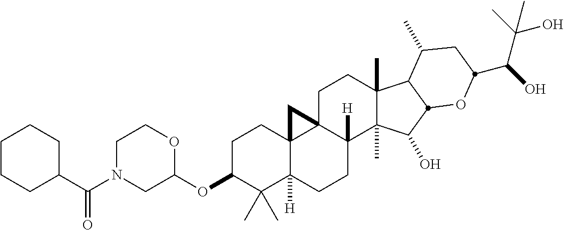 | 198 |
| 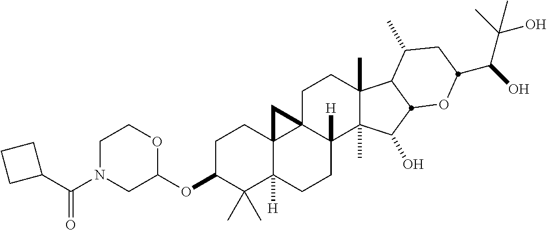 | 199 |
| 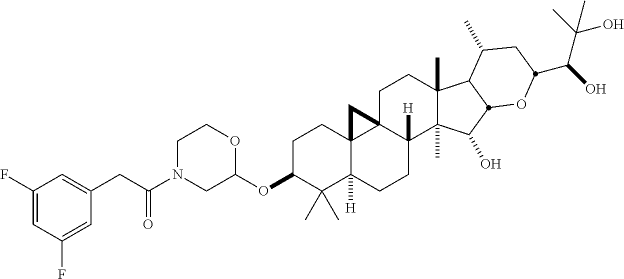 | 200 |
| 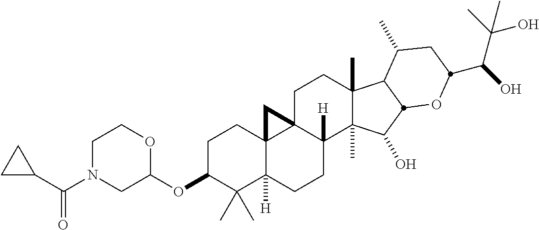 | 201 |
| 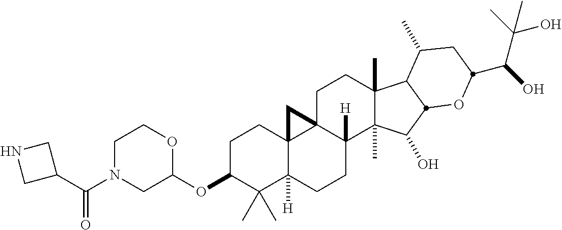 | 202 |
| 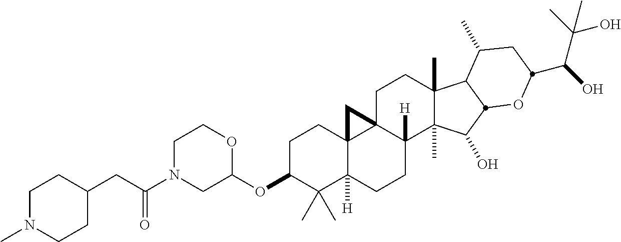 | 203 |
| 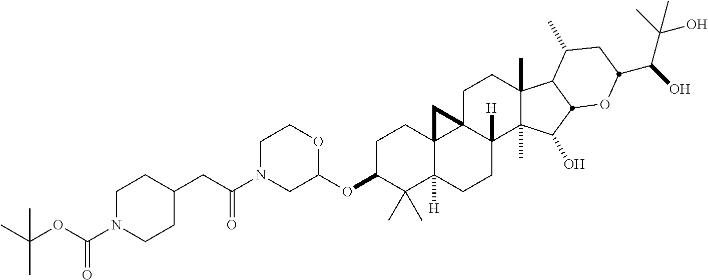 | 204 |
| 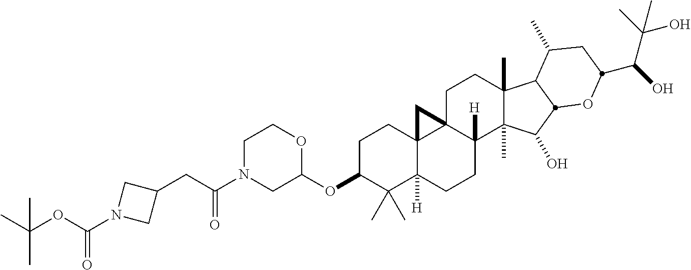 | 205 |
| 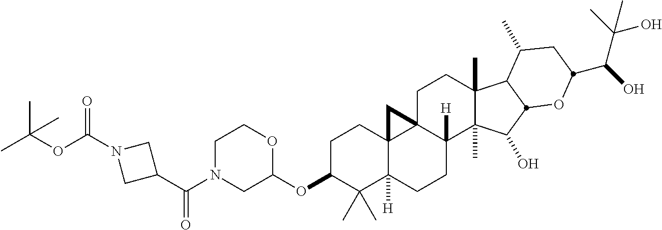 | 206 |
| 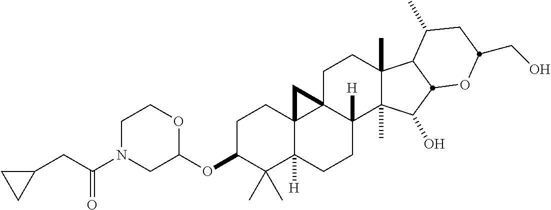 | 207 |

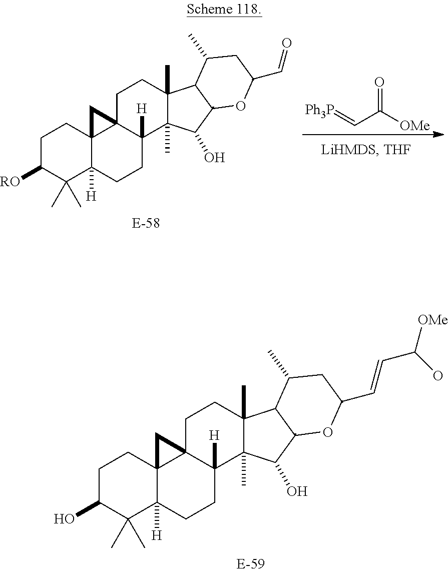

Procedure:

A slurry of (carbomethoxymethyl)triphenylphosphonium bromide (0.101 g, 0.243 mmol, 4.2 equiv.) in THF (3 mL) was cooled to 0° C. and LiHMDS (0.23 mL, 1 M in THF, 0.232 mmol, 4 equiv.) was added dropwise. The reaction was allowed to slowly warm to rt and the solids all went into solution. After 1 h, a solution of the aldehyde (0.025 g, 0.058 mmol) in THF (3 mL) was transferred to the ylide via syringe. The reaction was stirred at rt, monitoring progress by LC/MS. After 48 h, poured into CH₂Cl₂/H₂O, and separated layers. The aqueous layer was extracted with CH₂Cl₂, and then the combined organic layers were washed with brine and concentrated. The crude residue was purified via silica gel flash column chromatography, eluting with hexanes/ethyl acetate.

TABLE 14
Compounds

| | |
|---|---|
| 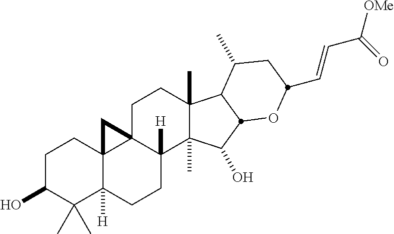 | 208 |
| 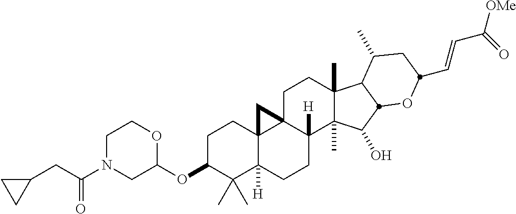 | 209 |

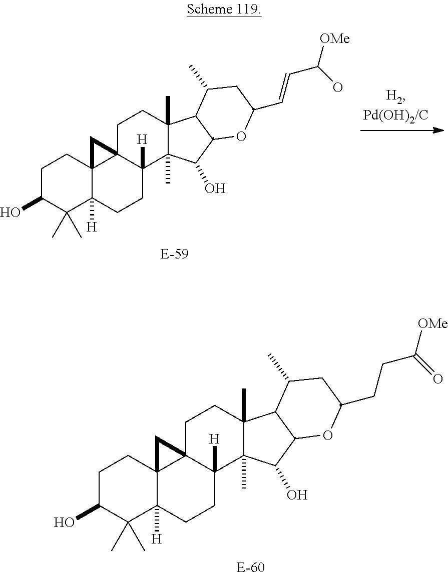

Procedure:

A solution of the α,β-unsaturated ester (0.1728 g, 0.355 mmol) in EtOAc (15 mL) and MeOH (1 mL) was degassed by bubbling N₂ through the solution, then was treated with Pd(OH)₂ (0.015 mg, 20% on carbon, wet). The reaction mixture was degassed again by bubbling N₂ through, then H₂ was bubbled through to saturate the solvent with H₂, and the solution was stirred at rt under an atmosphere of H₂. Stirred for 17 h, then filtered through Celite and concentrated the filtrate. The crude residue was purified via silica gel flash column chromatography eluting with CH₂Cl₂/MeOH.

TABLE 15
Compounds

| | |
|---|---|
| 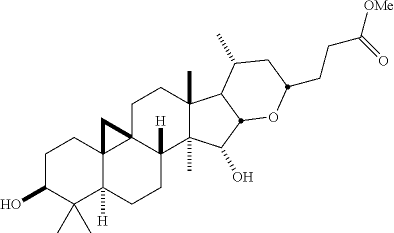 | 210 |
| 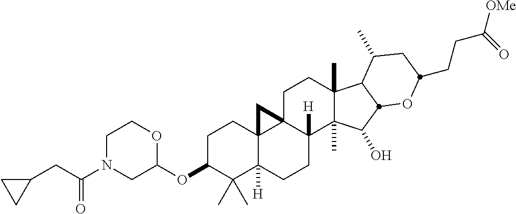 | 211 |

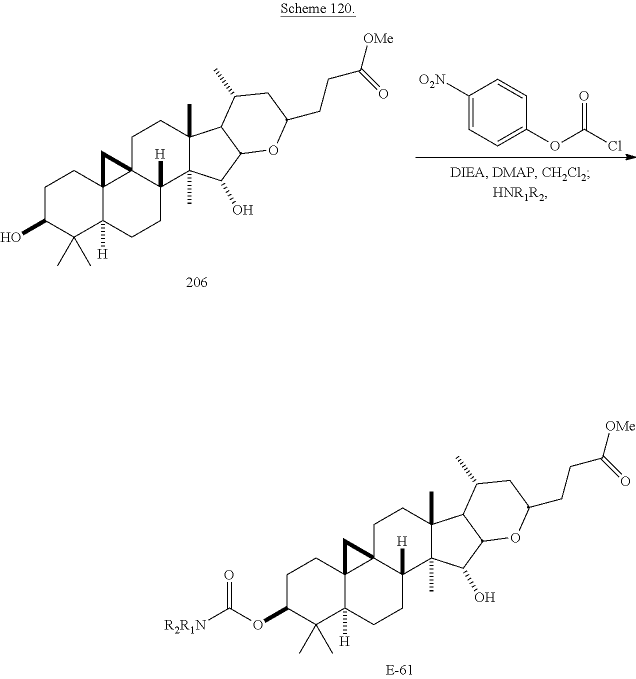

Procedure:

A solution of the C3-alcohol (0.050 g, 0.102 mmol) in CH₂Cl₂ (3 mL) was treated with diisopropylethylamine (0.11 mL, 0.614 mmol) followed by DMAP (0.013 g, 0.107 mmol) and 4-nitrophenylchloroformate (0.022 g, 0.107 mmol). The reaction was stirred at rt and monitored by TLC for disappearance of starting material. Once the majority of starting material was seen to be converted to product by TLC, the amine was added (0.204 mmol) and the reaction was monitored by LC/MS. After 1 h, the reaction mixture was loaded directly onto a silica gel column for flash purification, eluting with CH₂Cl₂/MeOH.

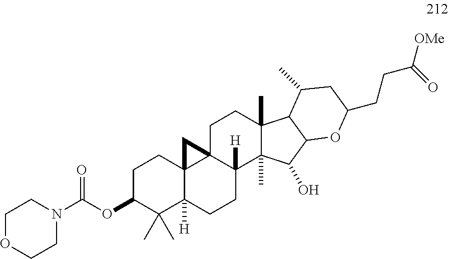

Compound:

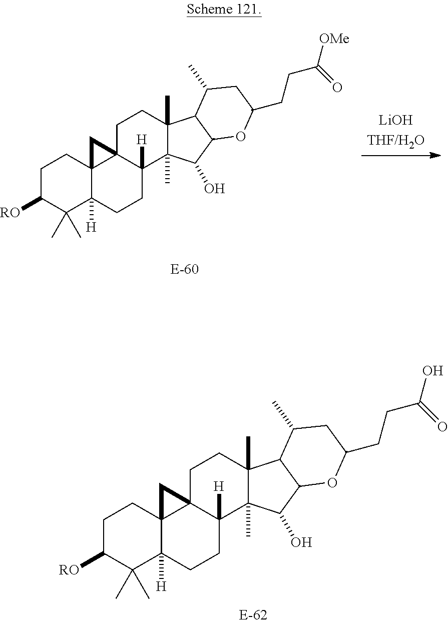

Procedure:

A solution of the methyl ester (0.017 g, 0.026 mmol) in THF (1.5 mL) and H₂O (0.5 mL) was treated with LiOH (0.0062 g, 0.26 mmol) and the reaction was stirred at rt, monitoring by LC/MS. After 2 h, full conversion of starting material to desired product was observed by LC/MS, so the reaction mixture was poured into Et₂O/H₂O and the layers were separated. The aqueous layer was acidified to pH z 2 with 1 M HCl (aq.), and was then extracted with Et₂O (×3). The combined organic layers were dried (MgSO₄), filtered, and concentrated to provide the crude product, which was purified using C18 reverse phase chromatography using CH₃CN/H₂O with 0.1% formic acid.

TABLE 16
Compounds

| | |
|---|---|
| 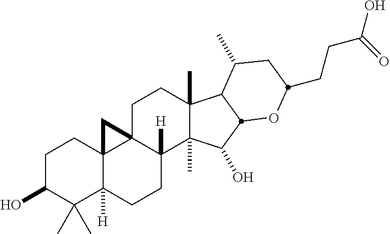 | 213 |
| 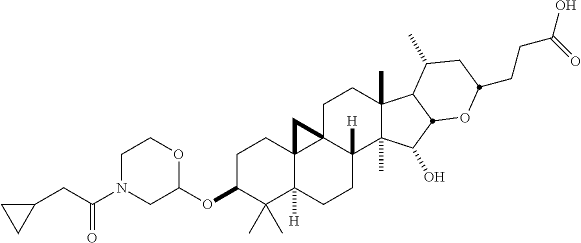 | 214 |
| 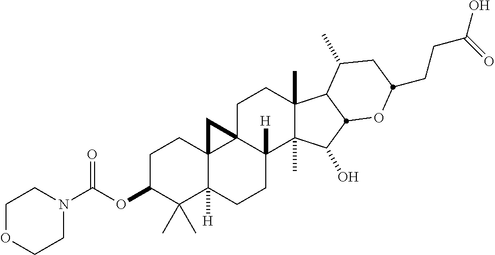 | 215 |

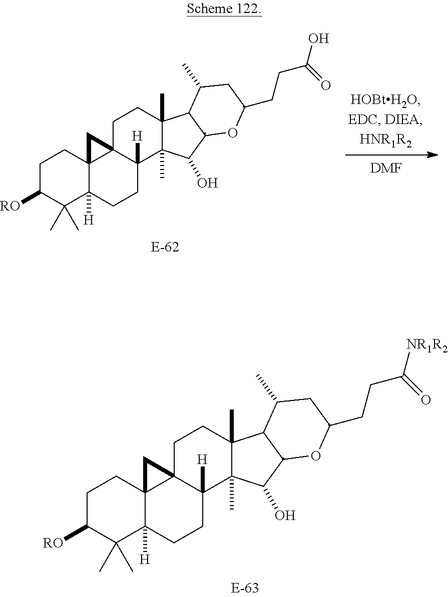

Procedure:

A solution of the carboxylic acid (0.0033 g, 0.0051 mmol) in DMF (2 mL) was treated sequentially with 1-hydroxybenzotriazolehydrate (HOBt-H₂O) (0.001 g, 0.0077 mmol), diisopropylethylamine (DIEA) (9 μL, 0.051 mmol), amine (0.0102 mmol), and 1-(3-dimethylaminopropyl)-3-ethylcarbodiimidehydrochloride (EDC) (0.0015 g, 0.0077 mmol). The reaction was stirred at rt, monitoring progress by LC/MS. When full conversion of starting material to desired product was observed, the reaction mixture was purified using C18 reverse phase chromatography, eluting with CH₃CN/H₂O with 0.1% formic acid.

TABLE 17
Compounds

| | |
|---|---|
| 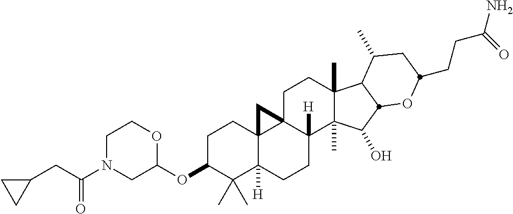 | 216 |
| 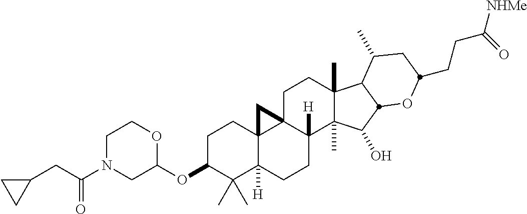 | 217 |
| 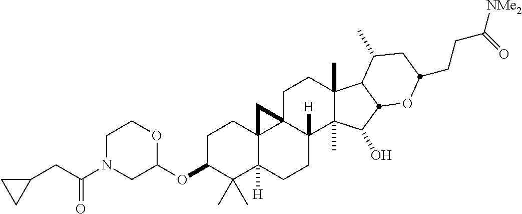 | 218 |
| 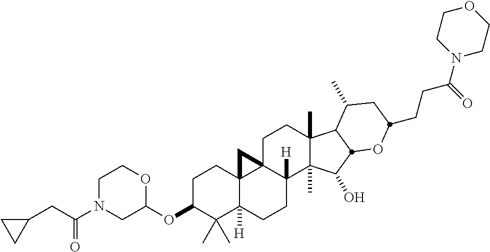 | 219 |

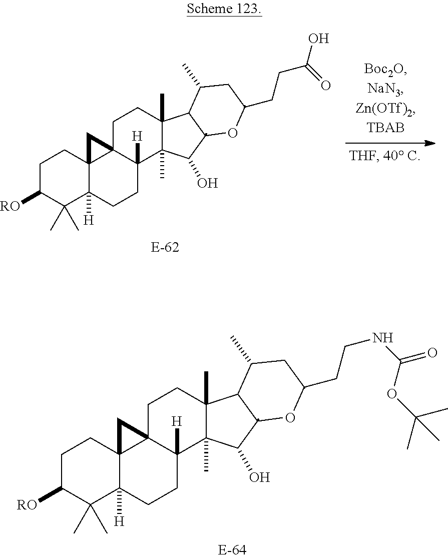

Procedure:

A solution of the carboxylic acid 209 (0.104 g, 0.162 mmol), NaN₃(0.037 g, 0.567 mmol), tetrabutylammonium bromide (0.008 g, 0.024 mmol), and Zn(OTf)₂ (0.009 g, 0.024 mmol) in THF (18 mL) was heated to 40° C., and then di-t-butyldicarbonate (0.06 mL, 0.243 mmol) was added, and the reaction was stirred at 40° C. overnight. Monitoring the reaction progress after 18 h by LC/MS showed a roughly 1:1 ratio of desired product to starting material, so added more di-t-butyldicarbonate (0.06 mL, 0.243 mmol) and continued stirring at 40° C. After 3 h, LC/MS shows no change, so proceeded with work up, adding 10% NaNO₂, stirring 20 min, then partitioning between EtOAc/H₂O, Separated layers, extracted the aqueous layer with EtOAc (×3), then washed the combined organic layers with saturated NH₄Cl (aq.), saturated NaHCO₃ (aq.), brine, and concentrated. Purified via silica gel flash column chromatography, eluting with hexanes/EtOAc.

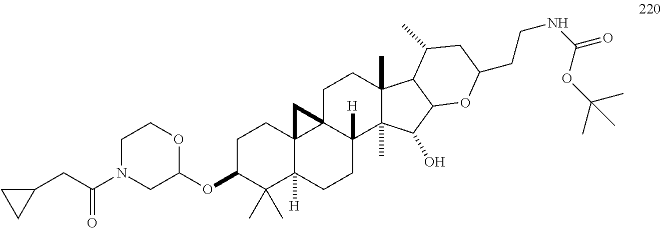

Compound:

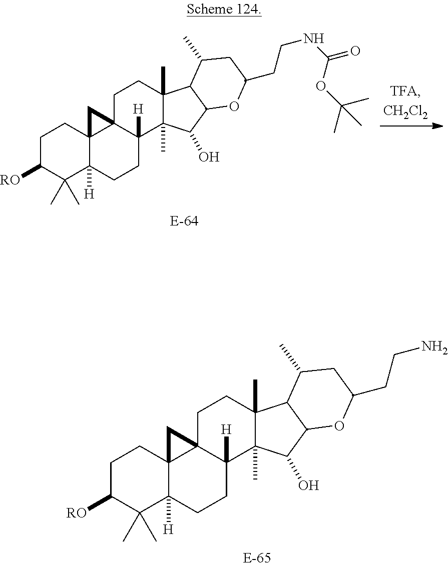

Procedure:

A solution of the N-Boc-carbamate (0.0245 g, 0.034 mmol) in CH₂Cl₂ (3 mL) was stirred at rt and treated with trifluoroacetic acid (0.5 mL). The reaction was stirred at rt, monitoring by LC/MS and TLC. After 5 min, the starting material has been consumed according to TLC and LC/MS, so poured into CH₂Cl₂/satd. NaHCO₃ (aq.) and separated layers. Extracted the aqueous layer with CH₂Cl₂, then washed the combined organic layers with satd. NaHCO₃ (aq.), brine, and concentrated. The crude product was purified using C18 reverse phase chromatography, eluting with CH₃CN/H₂O.

TABLE 18
Compounds

| | |
|---|---|
| 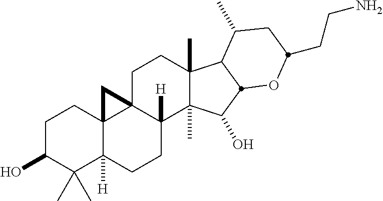 | 221 |
| 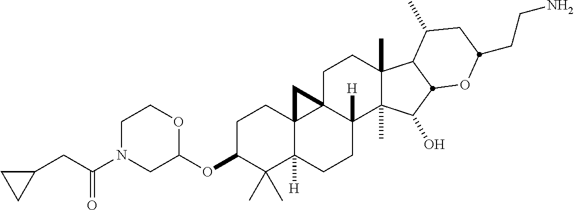 | 222 |

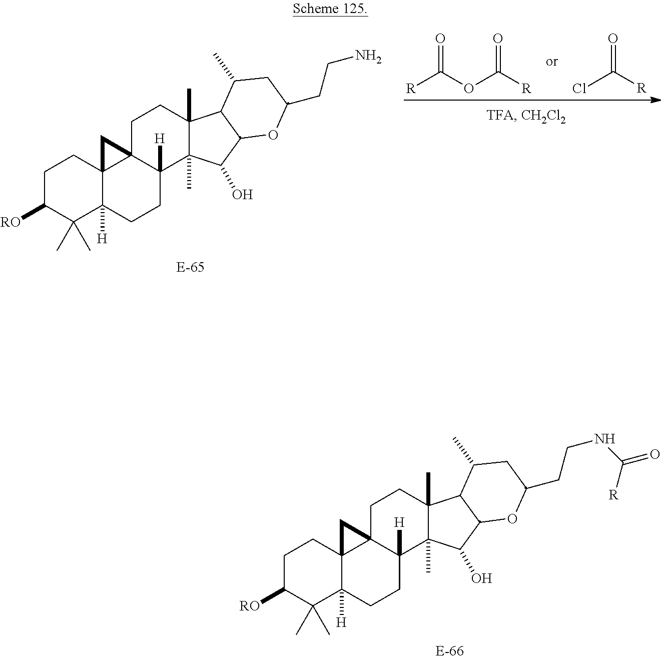

Procedure:

A solution of the primary amine (0.0077 mg) in CH₂Cl₂ (2.5 mL) was treated with triethylamine (10 μL, 0.075 mmol) followed by Ac₂O (1.2 μL, 0.012 mmol), and the reaction was stirred at RT, monitoring by LC/Ms. After 30 min, still see starting material by LC/MS, so added more Ac₂O (1.2 μL, 0.012 mmol), stirred overnight. Still a small amount of starting material after 10 h, so added more Ac₂O (1.2 μL, 0.012 mmol). After 30 min, no starting material detected by LC/MS, so poured into CH₂Cl₂/H₂O and separated layers. The organic layer was washed with 1 M HCl (aq.), brine, and concentrated. The crude residue was purified via C18 reverse phase chromatography, eluting with CH₃CN/H₂O with 0.1% formic acid.

TABLE 19
Compounds

| | |
|---|---|
| 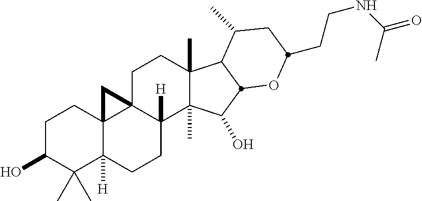 | 223 |
| 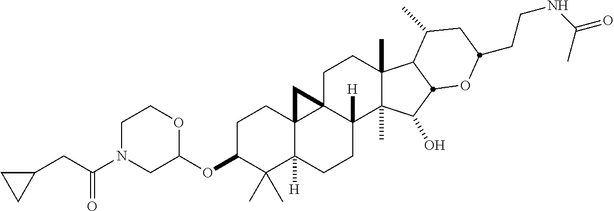 | 224 |
| 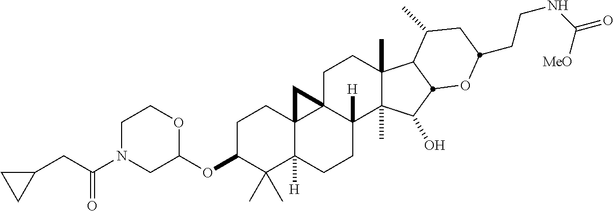 | 225 |

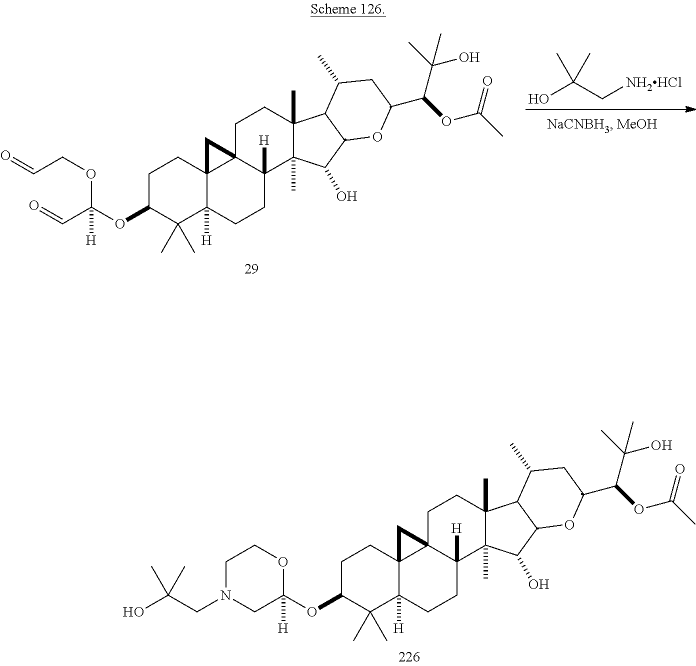

Procedure:

A solution of dialdehyde 29 (0.030 g, 0.047 mmol) in MeOH (3 mL) was treated with 1-amino-2-methyl propanol hydrochloride (0.015 g, 0.118 mmol) followed by sodium cyanoborohydride (0.009 g, 0.142 mmol). The reaction was stirred at RT and the progress was monitored by LC/MS. After 4 h, the LC/MS showed complete consumption of starting material, so the reaction mixture was loaded directly onto a 12 g C18-Biotage column and was purified using reverse phase chromatography, eluting with 10% to 100% CH₃CN in H₂O, to isolate 0.0129 g (39% yield) of the pure product. m/z [M+H]=690, m/z [M+Na]=712.

TABLE 20
Compounds

| | |
|---|---|
| 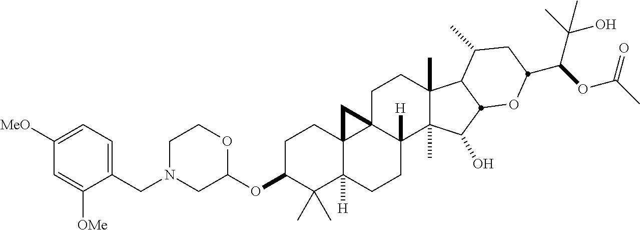 | 227 |
| 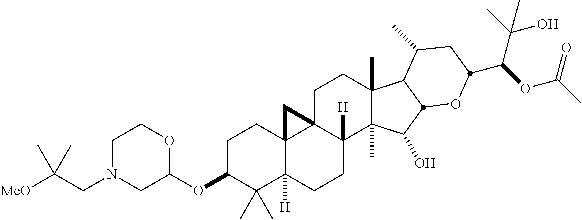 | 228 |
| 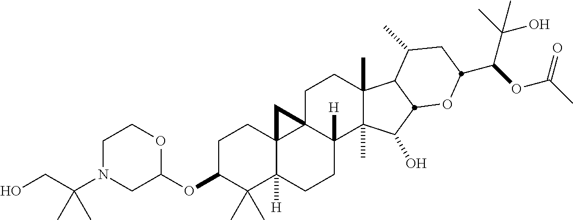 | 229 |
| 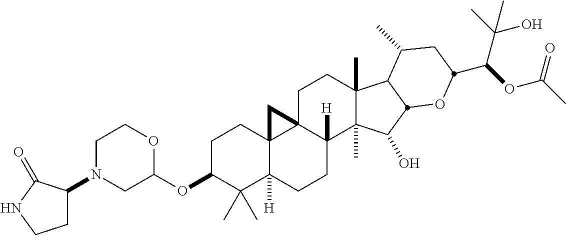 | 230 |
| 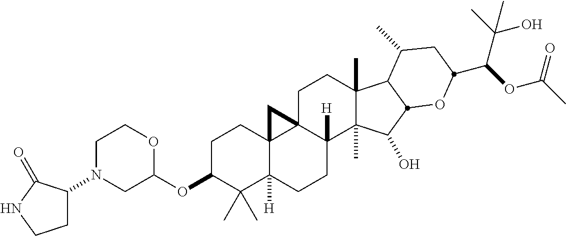 | 231 |
| 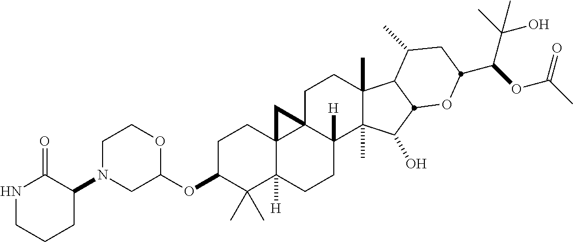 | 232 |
| 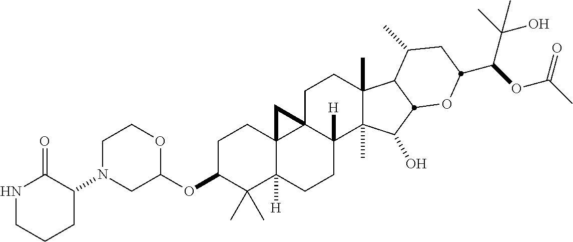 | 233 |
| 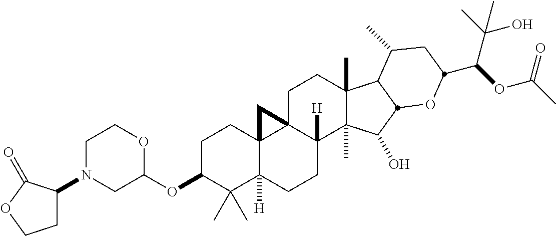 | 234 |
| 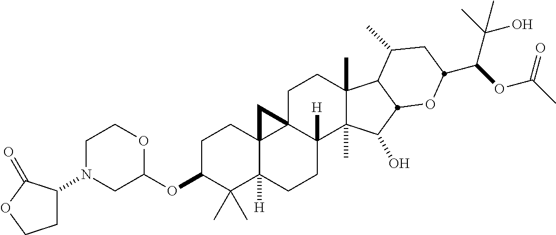 | 235 |
| 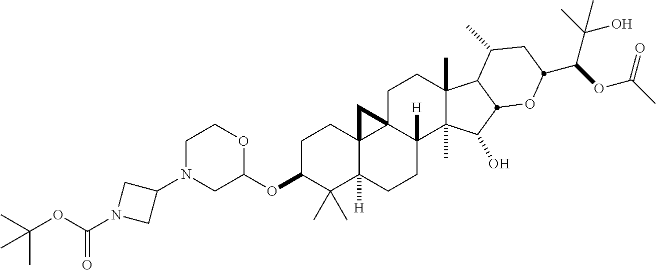 | 236 |
| 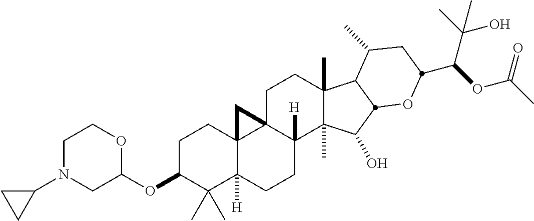 | 237 |
| 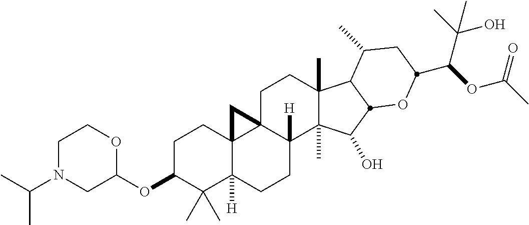 | 238 |
| 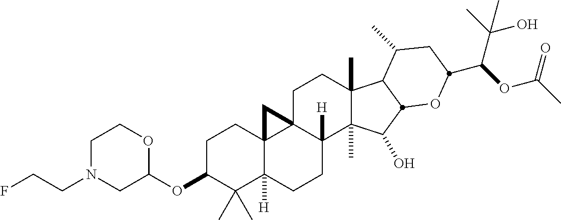 | 239 |
| 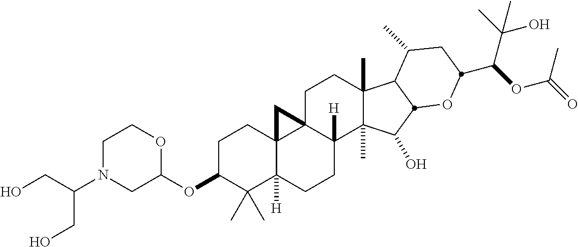 | 240 |

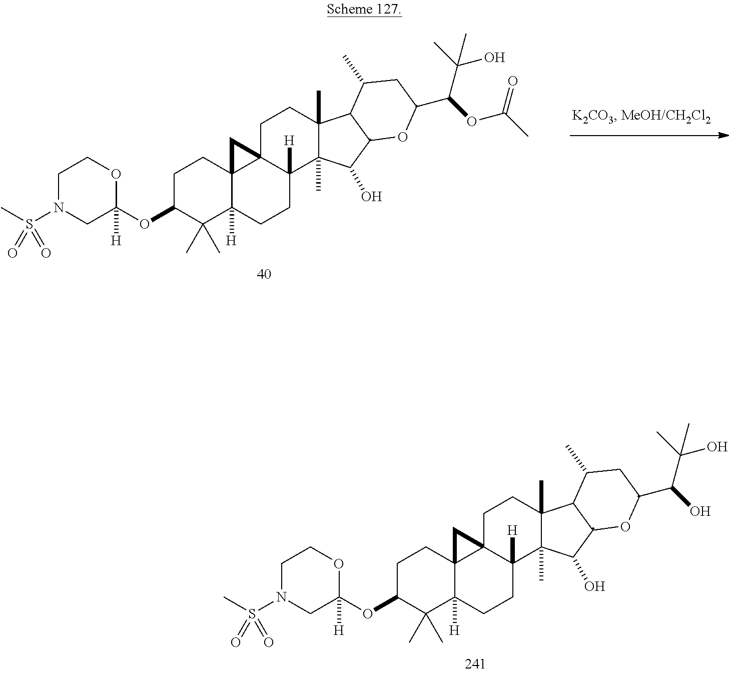

Procedure:

To a slurry of the acetate 40 (0.024 g, 0.034 mmol) in MeOH (3 mL) was added K₂CO₃ (0.024 g, 0.172 mmol), and the reaction was stirred at rt, monitoring progress by LC/MS. A small amount of CH₂Cl₂ (0.5 mL) was added to help solubilize the substrate. The reaction was stirred overnight (14 h), at which point the LC/MS showed full conversion to the desired product. The reaction mixture was poured into CH₂Cl₂ and saturated NaHCO₃ (aq.), and the layers were separated. The aqueous layer was extracted with CH₂Cl₂, and then the combined organic layers were washed with brine and concentrated. The crude residue was purified via flash column chromatography on a 10 g Biotage column, eluting with CH₂Cl₂/MeOH to isolate the desired product as a white solid m/z [M+H]=654, m/z [M+Na]=676.

TABLE 22
Compounds

| | |
|---|---|
| 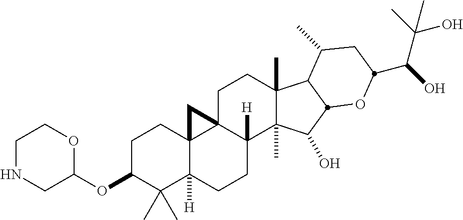 | 242 |
| 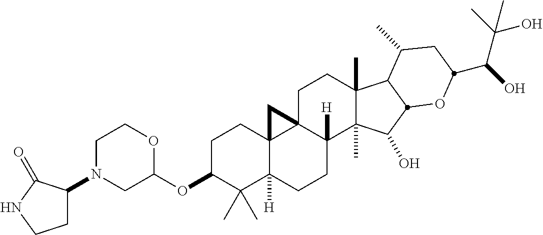 | 243 |
| 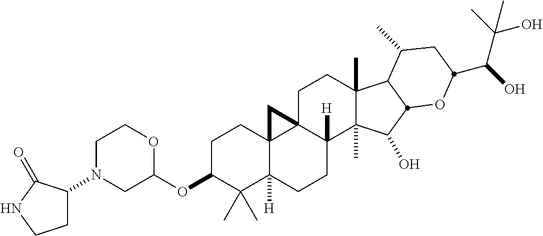 | 244 |
| 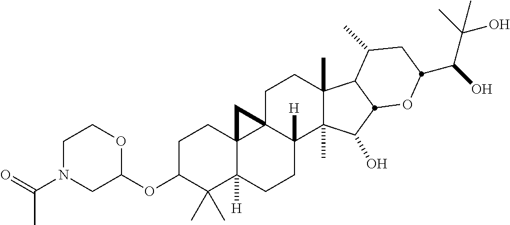 | 245 |
| 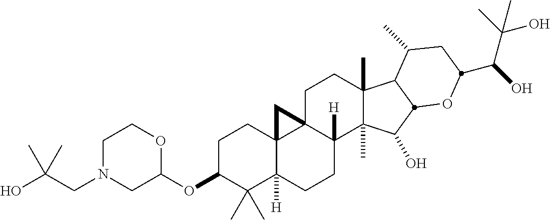 | 246 |
| 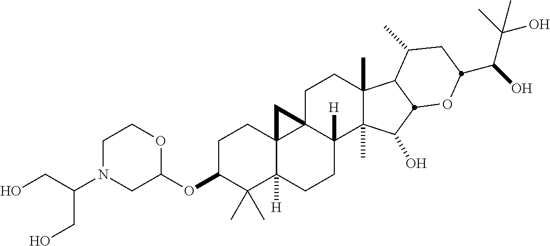 | 247 |
| 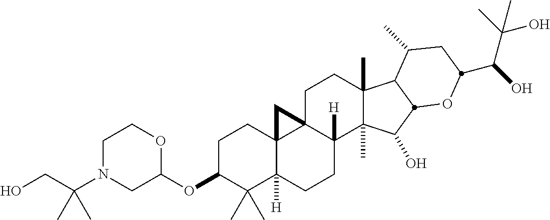 | 248 |
| 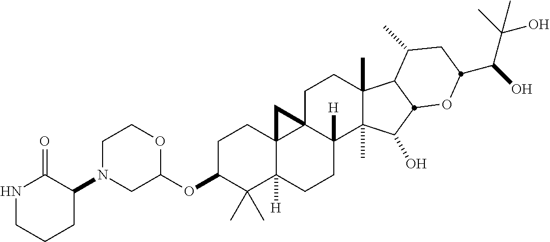 | 249 |
| 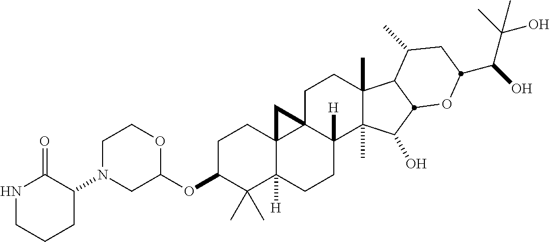 | 250 |
| 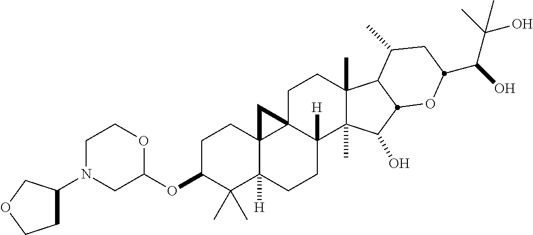 | 251 |
| 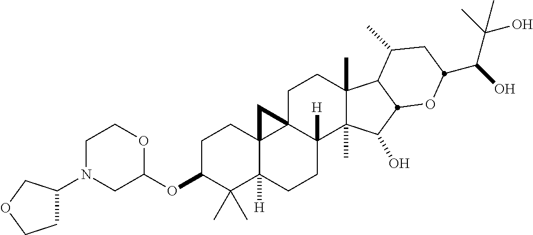 | 252 |
| 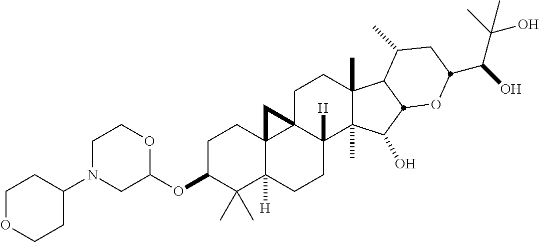 | 253 |
| 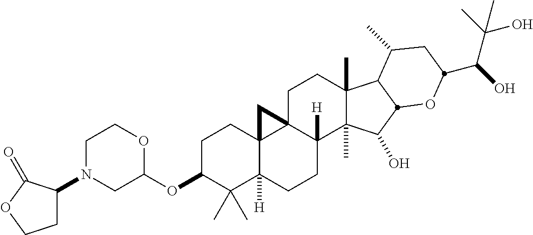 | 254 |
| 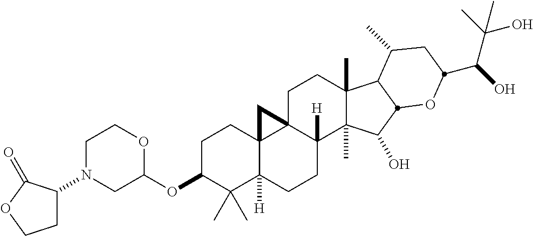 | 255 |
| 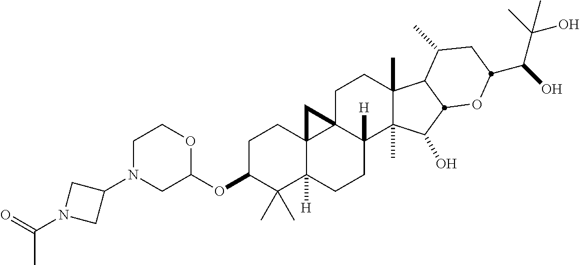 | 256 |
| 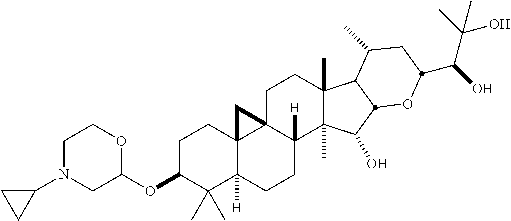 | 257 |
| 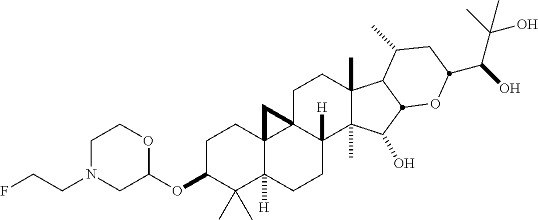 | 258 |

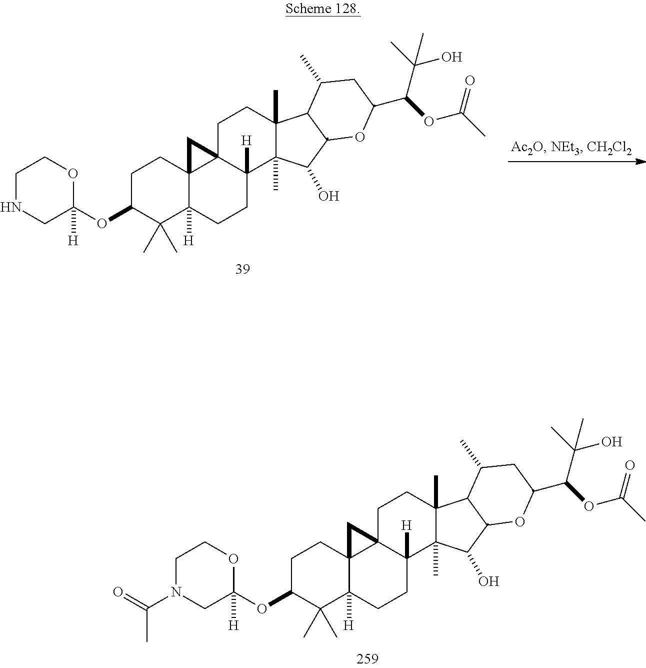

Procedure:

A solution of the amine 259 (0.0063 g, 0.010 mmol) in CH₂Cl₂ (3 mL) was treated with triethylamine (10 μL, 0.071 mmol) followed by Ac₂O (1 μL, 0.010 mmol). The reaction was stirred at rt, monitoring progress by LC/MS. After 20 min, LC/MS shows complete conversion to desired product, whereupon the reaction mixture was poured into CH₂Cl₂ and H₂O. After separating the layers, the organic layer was washed with 1 M HCl (aq.) and brine, then was concentrated under reduced pressure. The residue was taken up in MeOH and loaded onto a 12 g C-18 Biotage column and purified via reverse phase chromatography, eluting with 10% to 100% CH₃CN in H₂O. The desired acetamide was isolated as a pure white solid (4.9 mg, 73% yield) m/z [M+H]=660, m/z [M+Na]=682.

TABLE 23
Compounds

| | |
|---|---|
| 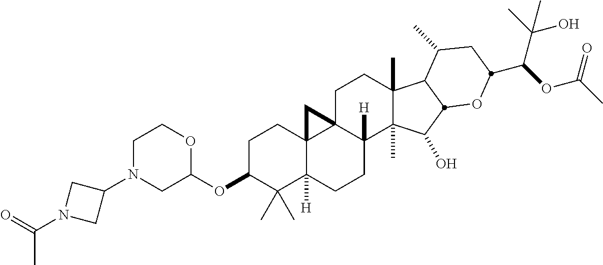 | 260 |
| 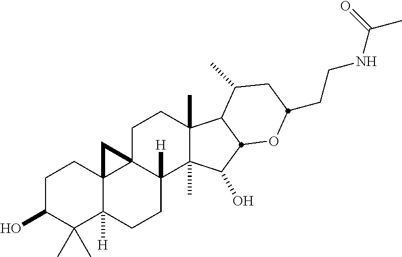 | 261 |
| 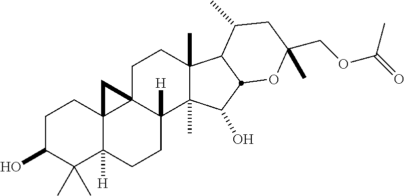 | 262 |

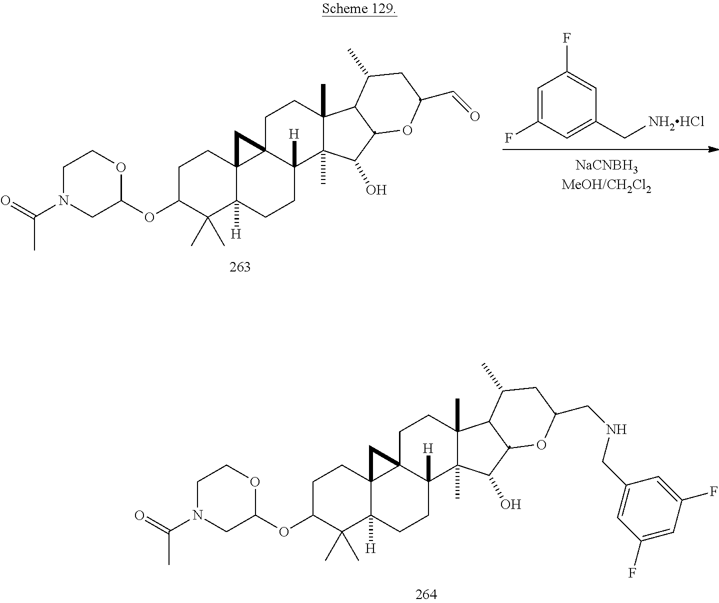

Procedure:

A solution of aldehyde 263 (0.016 g, 0.029 mmol) in MeOH (3 mL) and CH₂Cl₂ (1 mL) was treated with 3,5-difluorobenzylamine hydrochloride (0.013 g, 0.072 mmol) and the reaction was stirred for 90 min at rt, whereupon it was treated with sodium cyanoborohydride (0.0072 g, 0.115 mmol). The reaction was stirred at rt, monitoring progress via LC/MS. After 1 h, the LC/MS showed that the major component of the reaction mixture was the desired product, so the reaction mixture was concentrated down to about 1 mL of solvent and was loaded onto a 12 g C-18 Biotage column, purifying via reverse phase chromatography eluting with 10% to 100% CH₃CN in H₂O. The pure product was isolated as a white solid (5.0 mg, 26% yield) m/z [M+H]=685, m/z [M+Na]=707.

Compound:

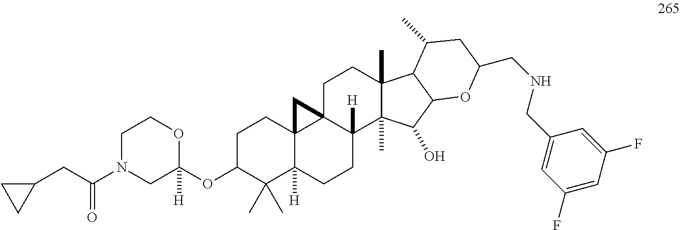

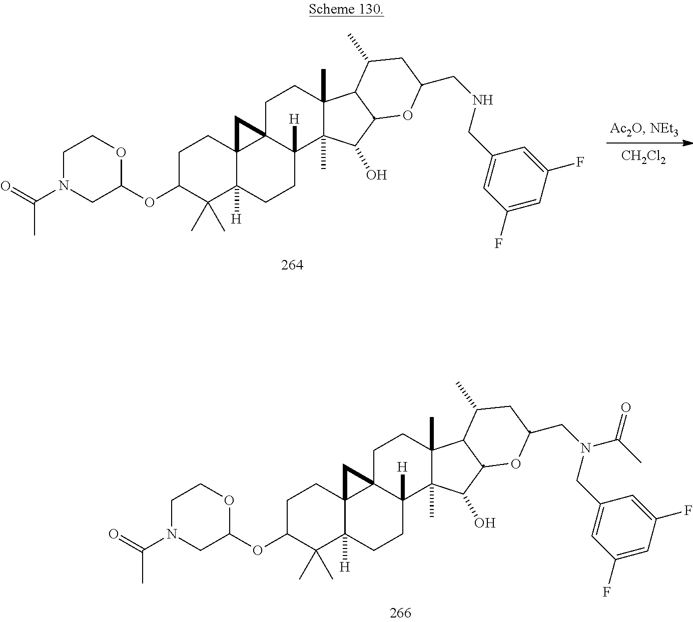

Procedure:

A solution of the amine (0.004 g, 0.006 mmol) in CH₂Cl₂ (3 mL) was treated with triethylamine (3.2 μL, 0.023 mmol) followed by Ac₂O (1.1 μL, 0.012 mmol) and the reaction was stirred at rt, monitoring progress via LC/MS. After 90 min, the starting material was consumed, so the reaction was poured into CH₂Cl₂ and H₂O and the layers were separated. The organic layer was washed with brine and concentrated under reduced pressure. The crude residue was purified via flash column chromatography using a 10 g Biotage column, eluting with CH₂Cl₂ and MeOH, providing the pure acetamide product as a waxy white solid m/z [M+H]=727, m/z [M+Na]=749.

Compound:

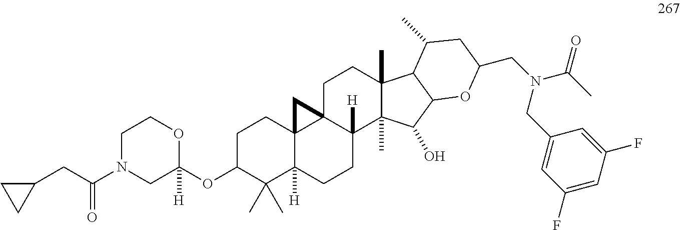

### Example 12

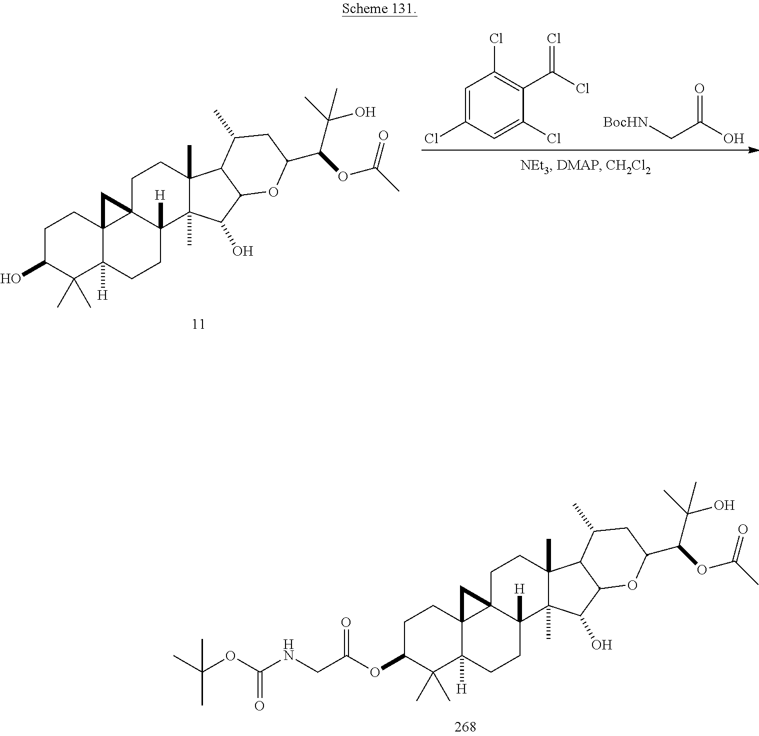

Procedure:

The triol (0.143 g, 0.268 mmol) was concentrated from toluene to ensure dryness, then was dissolved in CH₂Cl₂ (11 mL) under an atmosphere of N₂. The solution was treated in sequential order with N-Boc-glycine (0.049 g, 0.282 mmol), triethylamine (0.22 mL, 1.61 mmol), and 2,4,6-trichlorobenzoylchloride (84 μL, 0.537 mmol) and was stirred at rt. After 30 min, DMAP (0.039 g, 0.322 mmol) was added, causing the reaction to turn from yellow to orange. The reaction was stirred for 19 h, then was poured into CH₂Cl₂ and H₂O, and the layers were separated. The organic layer was washed with 1 M HCl (aq.), brine, dried (MgSO₄), filtered, and concentrated. The crude residue was purified via flash column chromatography eluting with CH₂Cl₂/MeOH, providing the pure acylated product (0.1402 g, 76% yield).

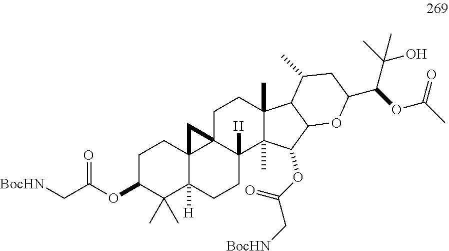

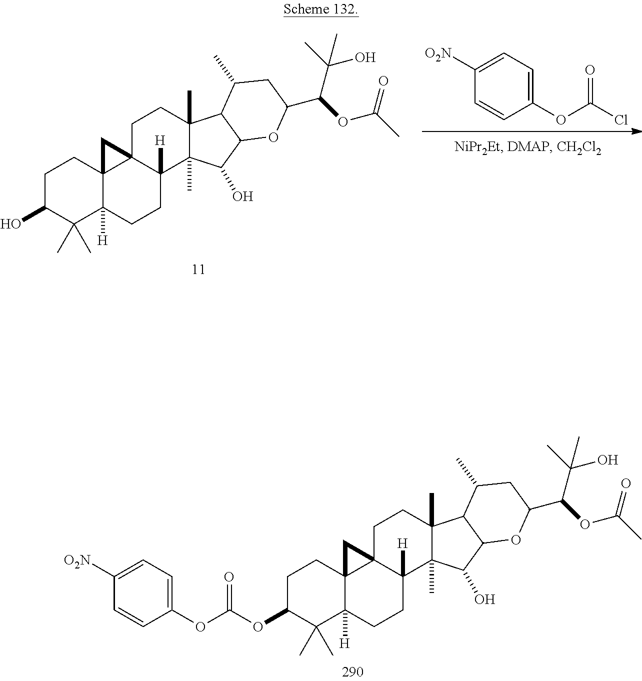

Procedure:

A solution of the triol (0.350 g, 0.657 mmol) in CH₂Cl₂ (7 mL) was treated sequentially with diisopropylethylamine (0.34 mL, 1.97 mmol), DMAP (0.084 g, 0.690 mmol), and then 4-nitrophenylchloroformate (0.139 g, 0.690 mmol) and the reaction was stirred at rt, following by TLC. After 90 min, loaded directly onto a 25 g Biotage flash column and purified, eluting with 20% to 100% EtOAc/Hex, providing 0.1989 g (43% yield) of the mixed carbonate product.

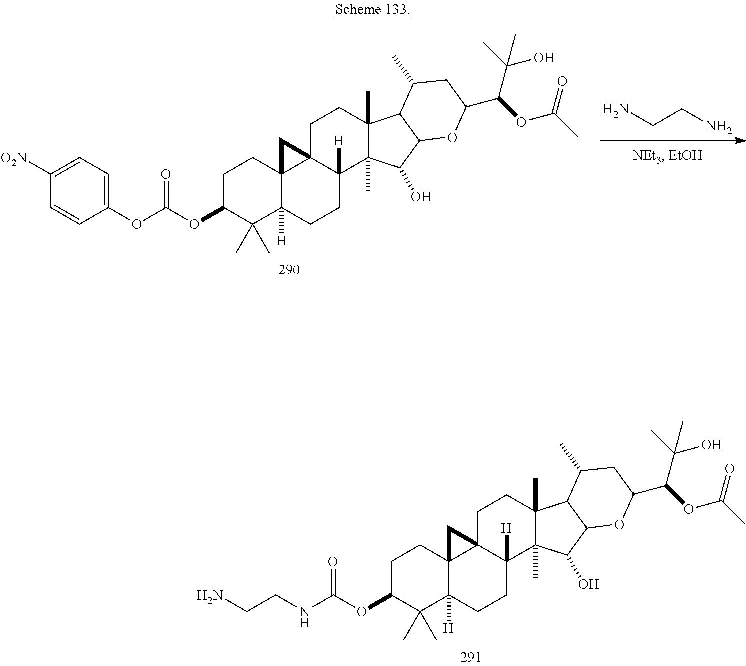

Procedure:

A solution of the mixed carbonate (0.037 g, 0.053 mmol) in EtOH (2 mL) was treated with ethylene diamine (35.4 μL, 0.53 mmol), followed by triethylamine (36.9 μL, 0.26 mmol), and the reaction was stirred at rt, monitoring progress by LC/MS. After 15 min, LC/MS shows complete conversion to desired product. The reaction mixture was filtered and then purified via reverse phase HPLC, eluting with 10% to 100% CH₃CN/H₂O with 0.1% formic acid.

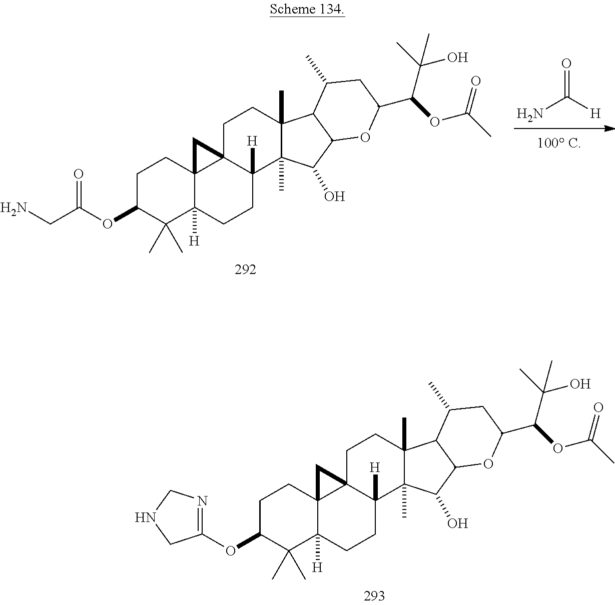

A solution of the amino ester (0.0147 g, 0.025 mmol) in formamide (1 mL) in a sealed tube under N₂ was heated to 100° C. and heated overnight. The reaction was then cooled to rt, filtered, and purified via reverse phase HPLC, eluting with 10% to 100% CH₃CN/H₂O with 0.1% formic acid.

TABLE 24
Compounds

| | |
|---|---|
| 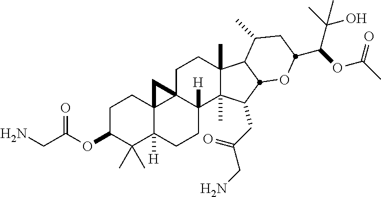 | 294 |
| 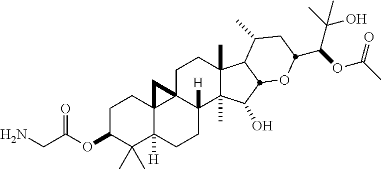 | 292 |

### Example 13

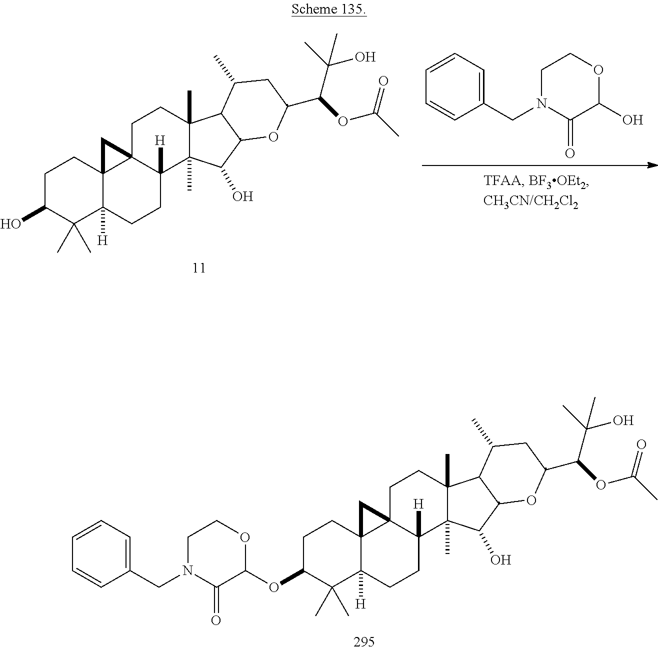

Procedure:

A slurry of benzyl lactam lactol (0.040 g, 0.197 mmol) in CH₃CN (2 mL) was cooled to 0° C. and trifluoroacetic anhydride (27.4 μL, 0.197 mmol) was added dropwise. After addition was completed, the cold bath was removed and the mixture was stirred at rt for 1 h. After 1 h, a solution of the triol (0.100 g, 0.188 mmol) in 1:1 CH₃CN:CH₂Cl₂ (4 mL) was then added dropwise, followed by BF₃OEt₂ (12.2 μL, 0.098 mmol). After observing no change in TLC after 90 min, an additional portion of BF₃OEt₂ (12.2 μL, 0.098 mmol) was added, and the reaction was stirred overnight. After 40 h total reaction time, the mixture was poured into CH₂Cl₂ and saturated NaHCO₃ (aq.), and the layers were separated. The organic layer was washed with brine, dried (MgSO₄), filtered, and concentrated. The pure product was isolated using reverse phase HPLC, eluting with 50% to 100% CH₃CN/H₂O with 0.1% formic acid m/z [M+H]=722, m/z [M+Na]=744.

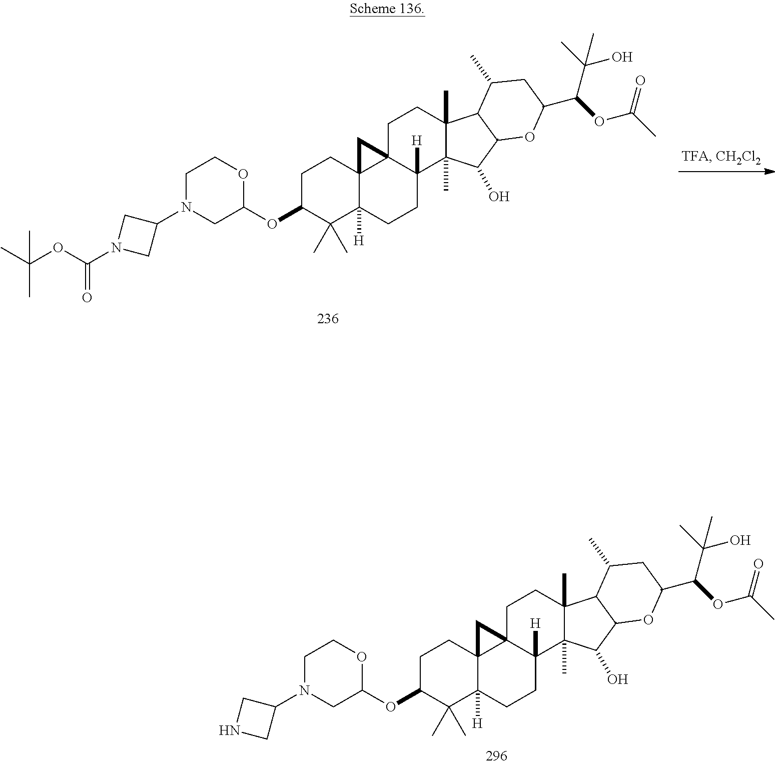

Procedure:

A solution of tert-butyl carbamate 236 (0.035 g, 0.045 mmol) in CH₂Cl₂ (2 mL) was treated with trifluoroacetic acid (1 mL) and the reaction was stirred at rt, monitoring by LC/MS. After 10 min, the LC/MS shows complete conversion of the starting material to the desired product. The reaction mixture was poured into CH₂Cl₂ and saturated NaHCO₃ (aq.), the layers were separated, and the organic layer was washed with saturated NaHCO₃ (aq.), brine, dried (MgSO₄), filtered, and concentrated. The crude residue was purified via flash column chromatography through a short plug of silica gel, eluting with 10% MeOH in CH₂Cl₂, providing 0.017 g of the pure amine (57% yield). The hydrochloride salt was prepared by treating a solution of the free amine (0.005 g) in EtOH (2 mL) and CH₂Cl₂ with 1 M HCl (1.7 μL, 1 equiv.) and concentrating under reduced pressure to provide the white hydrochloride salt in quantitative yield.

TABLE 25
Compounds

| | |
|---|---|
| 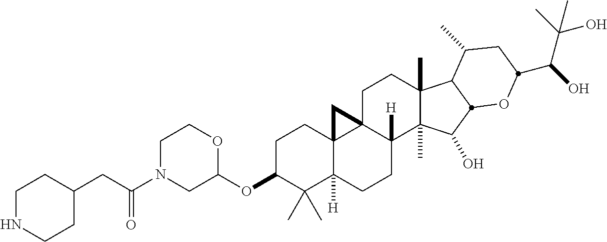 | 297 |
| 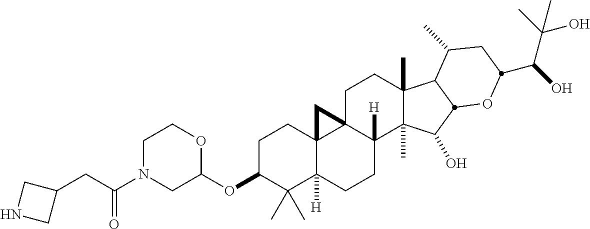 | 298 |
| 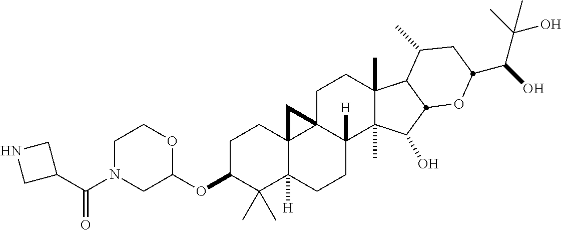 | 299 |
| 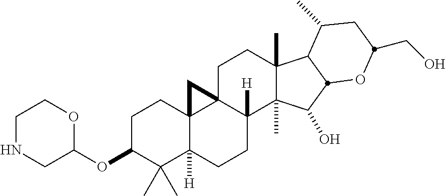 | 300 |

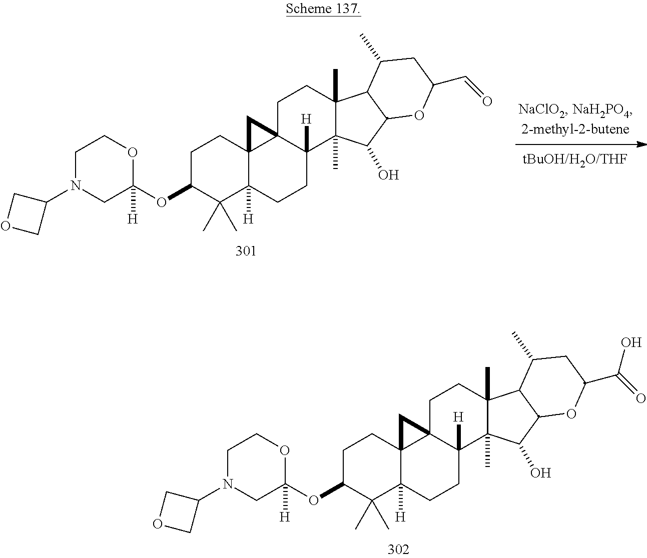

Procedure:

A solution of the aldehyde (0.100 g, 0.175 mmol) in tert-butanol (3 mL) and H₂O (1 mL) was treated with 2-methyl-2-butene (3 mL, 2 M in THF, 6.30 mmol), followed by NaH₂PO₄ (0.252 g, 2.10 mmol) and NaClO₂ (0.111 g, 1.22 mmol). The reaction was stirred at rt, monitoring progress by LC/MS. After 90 min, poured into CH₂Cl₂ and H₂O and separated layers. The aqueous layer was extracted with CH₂Cl₂, then the combined organic layers were washed with brine, dried (MgSO₄), filtered, and concentrated. Purification was carried out using a 30 g C-18 Biotage reverse phase column, eluting with 10% to 100% CH₃CN/H₂O to provide 0.041 g pure acid (40% yield) m/z [M+H]=588.

Compound:

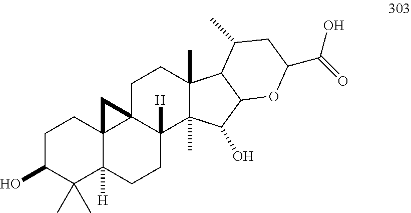

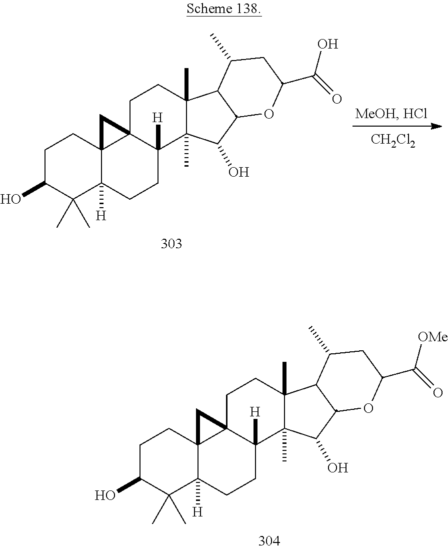

Procedure:

A solution of the carboxylic acid (0.110 g, 0.246 mmol) in MeOH (5 mL) and CH₂Cl₂ (2 mL) was treated with concentrated HCl (4 drops) and the reaction was stirred at rt, monitoring progress by LC/MS. After 90 min reaction is complete, so poured into CH₂Cl₂ and saturated NaHCO₃ (aq.) and separated layers. The aqueous layer was extracted with CH₂Cl₂, then the combined organic layers were washed with brine and concentrated. The crude residue was purified via Biotage flash column chromatography, eluting with 0% to 8% MeOH/CH₂Cl₂ to provide 0.101 g (89%) pure methyl ester m/z [M+Na]=483.

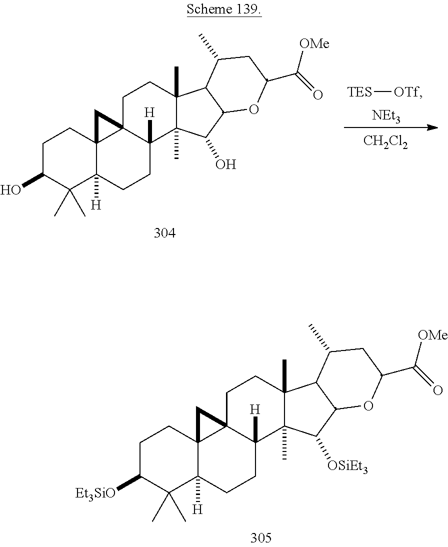

Procedure:

A solution of the diol (0.101 g, 0.219 mmol) in CH₂Cl₂ (8 mL) was cooled to 0° C. and treated with triethylamine (0.31 mL, 2.19 mmol) followed by triethylsilyltrifluoromethanesulfonate (TES-OTf) (0.12 mL, 0.548 mmol) and the reaction was allowed to slowly warm to rt, following progress by TLC. After 1 h, TLC showed starting material remaining, so additional TES-OTf (0.06 mL, 0.274 mmol) was added. After 30 min more, the reaction was complete, so the reaction was poured into CH₂Cl₂ and saturated NaHCO₃ (aq.) and the layers were separated. The organic layer was washed with saturated NaHCO₃ (aq.), water (×2), brine, and concentrated. The crude residue was purified via Biotage flash column chromatography, eluting with 10% to 15% EtOAc/Hex, providing a quantitative yield of the bis-silyl ether.

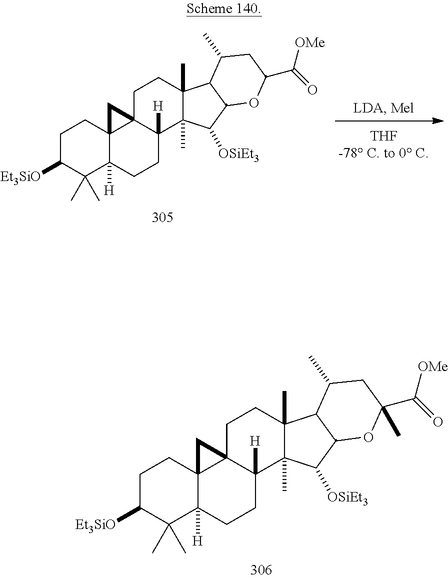

Procedure:

In a flame-dried flask under N₂, a solution of diisopropylamine (0.05 mL, 0.35 mmol) in THF (0.5 mL) was cooled to 0° C. and n-BuLi (0.13 mL, 2.5 M in hexanes, 0.33 mmol) was added dropwise. The reaction was stirred at 0° C. for 5 min, then at rt for 15 min, and then was cooled to −78° C. A solution of the methyl ester (0.150 g, 0.218 mmol) in THF (2 mL) was added dropwise over 5 min, the reaction was stirred at −78° C. for 1 h, and then iodomethane (68 μL, 1.09 mmol) was added dropwise. After stirring for 90 min at −78° C., the reaction was stirred at 0° C. for 30 min, whereupon the TLC showed complete consumption of starting material. The reaction was quenched with satd. NH₄Cl (aq.) and poured into Et₂O/H₂O and the layers were separated. The aqueous layer was extracted with Et₂O and the combined organic layers were washed with brine, dried (MgSO₄), filtered, and concentrated. The crude residue was purified via Biotage flash column chromatography, eluting with EtOAc/Hex to provide a quantitative yield of the alkylation product.

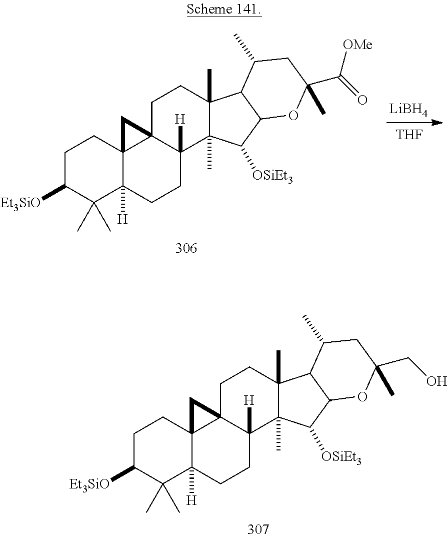

Procedure:

A solution of the ester (0.038 g, 0.054 mmol) in THF (3 mL) was treated with LiBH₄ (0.11 mL, 2 M in THF, 0.22 mmol) and the reaction was stirred at rt, monitoring progress by TLC. After 16 h, TLC shows roughly a ratio of 1:1 starting material:desired product, so added additional LiBH₄ (0.11 mL, 2 M in THF, 0.22 mmol), and then after 3 h more LiBH₄ (0.11 mL, 2 M in THF, 0.22 mmol) was used to push the reaction to completion. After 4 h more, the reaction was rendered complete and was poured into EtOAc/H₂O. The layers were separated, and the organic layer was washed with brine and concentrated. The crude residue was purified via flash column chromatography in EtOAc/Hex to provide 0.0295 g (81% yield) of the resulting alcohol.

TABLE 32
Compounds

| | |
|---|---|
| 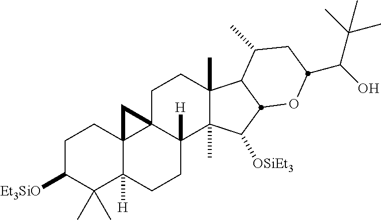 | 308 |
| 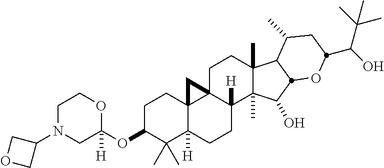 | 309 |

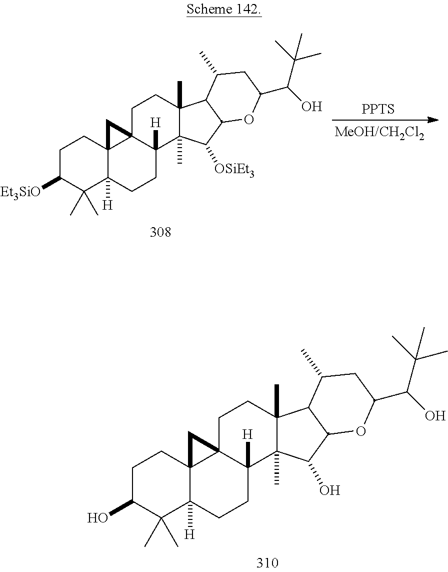

Procedure:

A solution of the bis-silyl ether 308 (0.009 g, 0.012 mmol) in CH₂Cl₂ (1 mL) and MeOH (1 mL) was treated with a catalytic amount of pyridinium p-toluensulfonate (PPTS) and the reaction was stirred at rt, monitoring by TLC. After 30 min, the starting material had been consumed, so concentrated under reduced pressure and purified via flash column chromatography, eluting with CH₂Cl₂/MeOH to provide 0.0056 g (92% yield) m/z [M+Na]=511.

TABLE 33
Compounds

| | |
|---|---|
| 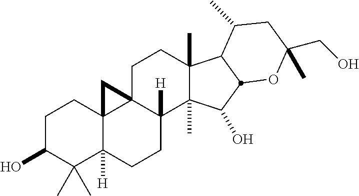 | 311 |
| 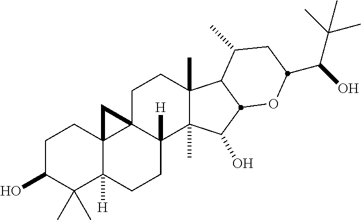 | 312 |
| 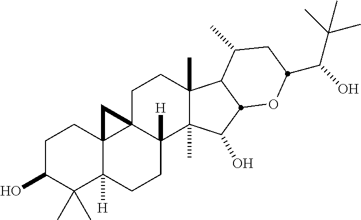 | 313 |
| 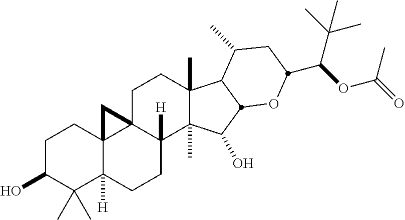 | 314 |

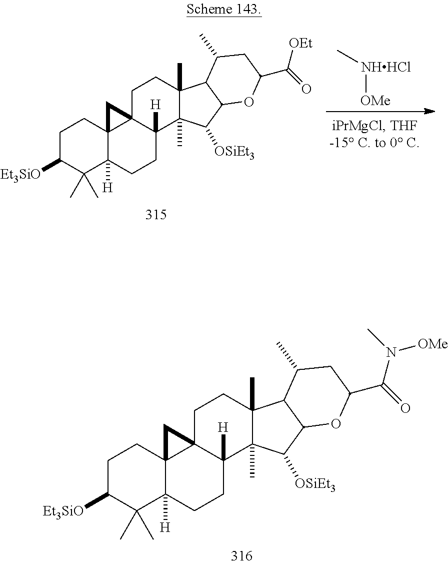

Procedure:

In a flame-dried flask under N₂, a solution of ester NF-14 (0.215 g, 0.306 mmol) in THF (6 mL) was cooled to −15° C. and N,O-dimethylhydroxylamine hydrochloride (0.119 g, 1.22 mmol) was added, followed by dropwise addition of iPrMgCl (1.8 mL, 2 M in Et₂O, 3.67 mmol) over 10 min. The reaction was slowly allowed to warm to 0° C. After 2 h the reaction was complete by TLC and was quenched with saturated NH₄Cl (aq.), then poured into EtOAc/H₂O. The layers were separated, the aqueous layer was extracted with EtOAc (×2), the combined organic layers were washed with brine, and concentrated. Purification was carried via flash column chromatography, eluting with 10% to 35% EtOAc/Hex to provide 0.1823 g (83% yield) of the Weinreb Amide product.

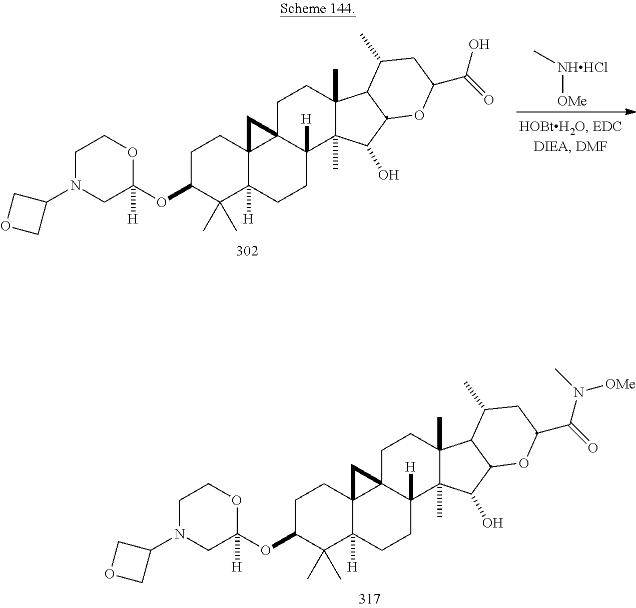

Procedure:

A solution of the carboxylic acid (0.147 g, 0.250 mmol) in DMF (4 mL) was treated sequentially with 1-hydroxybenzotriazolehydrate (HOBt-H₂O) (0.064 g, 0.50 mmol), diisopropylethylamine (DIEA) (0.6 mL, 3.0 mmol), amine (0.05 g, 0.50 mmol), and 1-(3-dimethylaminopropyl)-3-ethylcarbodiimidehydrochloride (EDC) (0.095 g, 0.500 mmol). The reaction was stirred at rt, monitoring progress by LC/MS. After 17 h, starting acid still remained, so additional HOBt-H₂O (0.032 g, 0.25 mmol), DIEA (0.3 mL, 1.0 mmol), and EDC (0.047 g, 0.25 mmol) were added. After 5 h, full conversion to product was observed, so poured into CH₂Cl₂/H₂O and separated layers. The organic layer was washed with 1 M HCl (aq.), saturated NaHCO₃ (aq.), brine, and concentrated. Purification was performed using a 30 g C-18 Biotage column via reverse phase chromatography, eluting with 15% to 100% CH₃CN/H₂O, providing 0.140 g (89% yield) of the Weinreb Amide product.

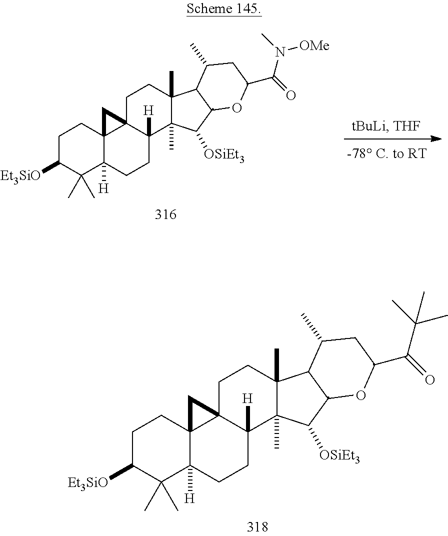

Procedure:

In a flame-dried flask under N₂, a solution of the Weinreb Amide 316 (0.050 g, 0.070 mmol) was cooled to −78° C. and treated with t-BuLi (0.12 mL, 1.7 M in pentane, 0.21 mmol), and the reaction was stirred at −78° C. and followed by TLC. After 70 min the reaction was nearly complete by TLC, so removed from cooling bath, stirred at rt for 15 min, then quenched with saturated NH₄Cl (aq.) and poured into Et₂O/H₂O. After separating layers, the organic layer was washed with brine, dried (MgSO₄), filtered, and concentrated. The crude residue was purified via flash column chromatography, eluting with 0% to 5% MeOH/CH₂Cl₂ to provide 0.041 g (82% yield) of the ketone product.

TABLE 34
Compounds

| | |
|---|---|
| 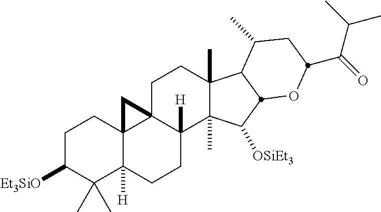 | 319 |
| 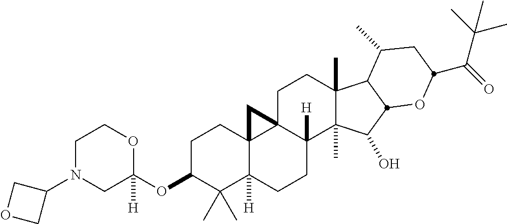 | 320 |

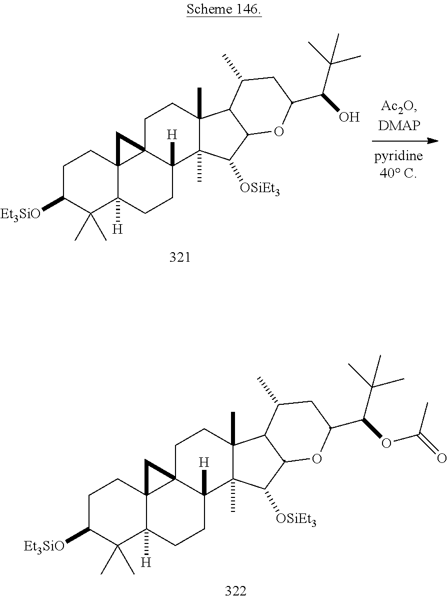

Procedure:

A solution of the alcohol (0.014 g, 0.020 mmol) in pyridine (1.5 mL) was treated with DMAP (0.0029 g, 0.023 mmol) and Ac₂O (3.7 μL, 0.040 mmol) and the reaction was heated to 40° C., monitoring by TLC. After 1 h, TLC showed only SM, so additional DMAP (0.002 g) and Ac₂O (5 μL) were added. After 5 h, TLC shows a new spot. Added more Ac₂O (2 μL) and heated at 40° C. overnight. TLC after 20 h showed the reaction to be nearly complete, so poured into CH₂Cl₂/H₂O and separated layers. The organic layer was washed with brine and concentrated. The crude residue was purified by flash column chromatography to provide the acetate product.

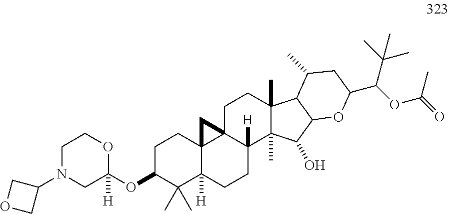

Compound:

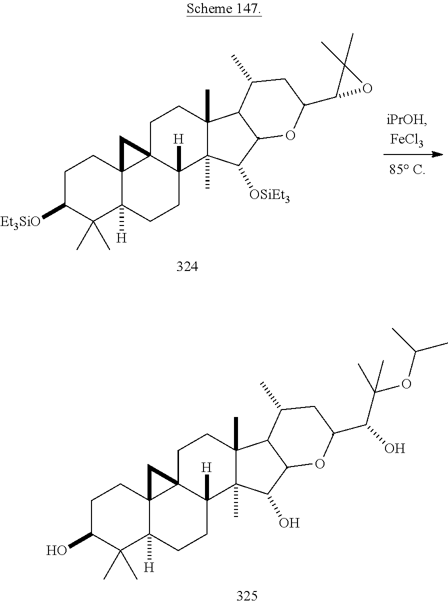

Procedure:

A slurry of epoxide (0.029 g, 0.041 mmol) in iPrOH (2.5 mL) was treated with FeCl₃ (1.3 mg, 0.008 mmol), and the reaction was heated to 85° C. and stirred 14 h. TLC analysis shows complete consumption of starting material and LC/MS shows ether formation along with silyl ether cleavage. Concentrated under reduced pressure, then purified via flash column chromatography in 10% to 90% EtOAc/Hex to provide 0.009 g (41% yield) of the ether.

### Compound:

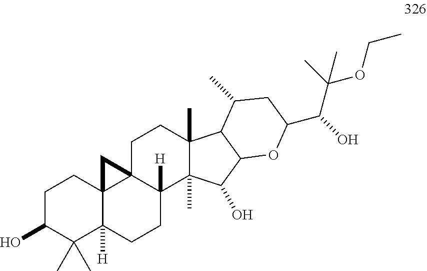

TABLE 35
Compounds

| | |
|---|---|
| 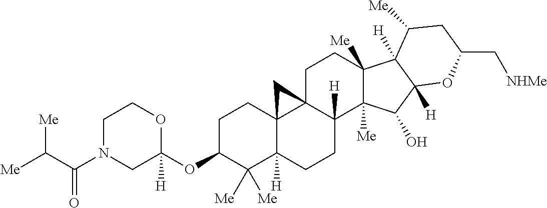 | 327 |
| 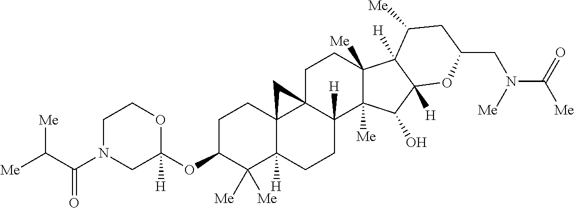 | 328 |
| 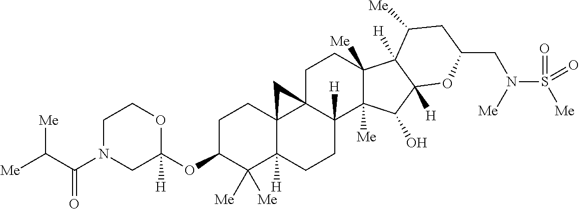 | 329 |
| 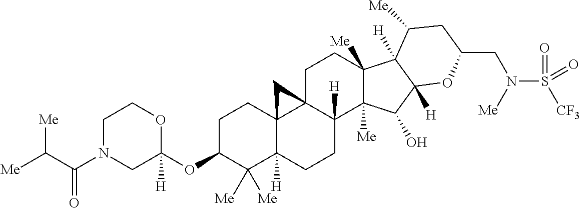 | 330 |
| 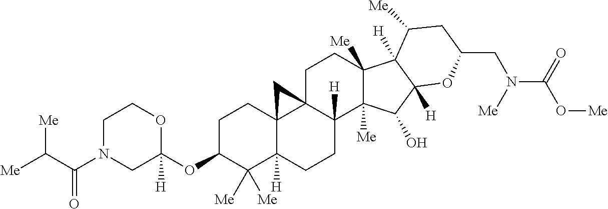 | 331 |
| 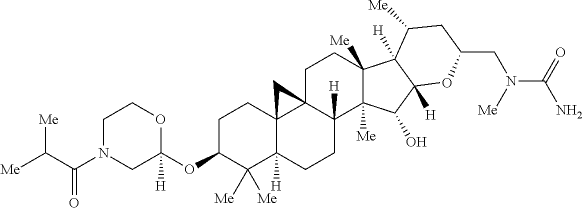 | 332 |
| 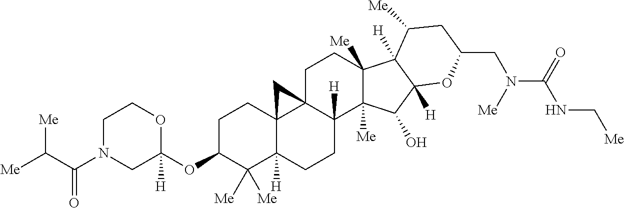 | 333 |
| 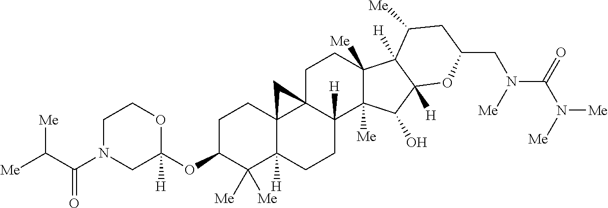 | 334 |
| 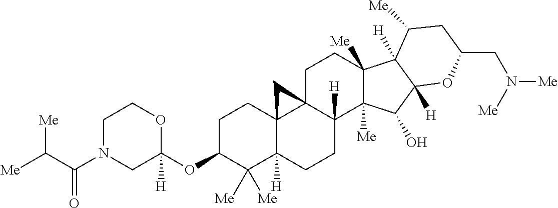 | 335 |
| 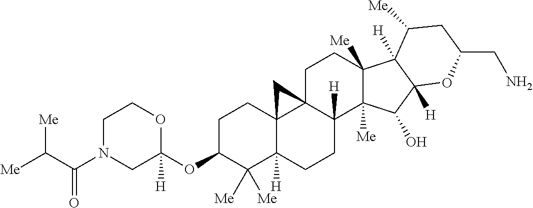 | 336 |
| 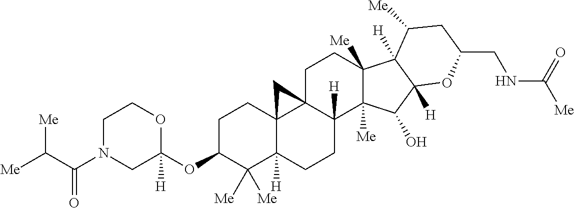 | 337 |
| 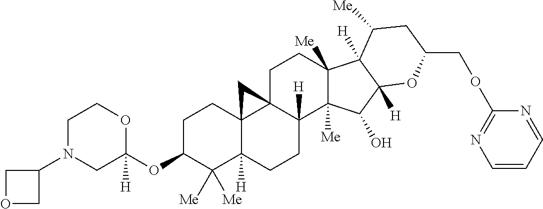 | 338 |
| 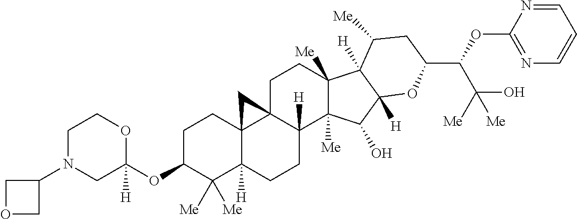 | 339 |
| 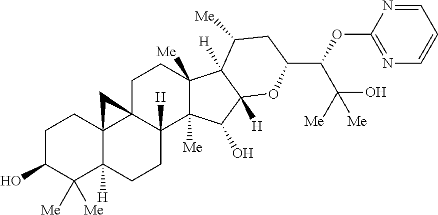 | 340 |
| 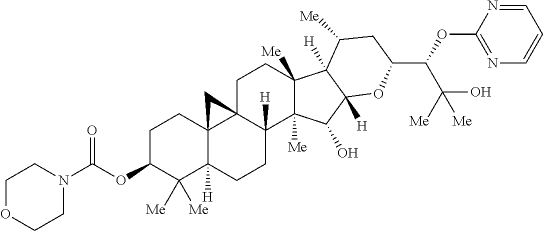 | 341 |
| 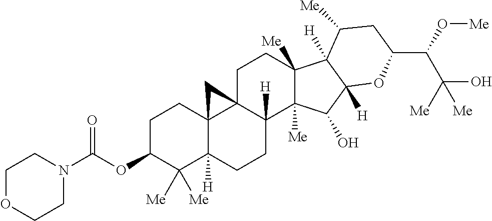 | 342 |
| 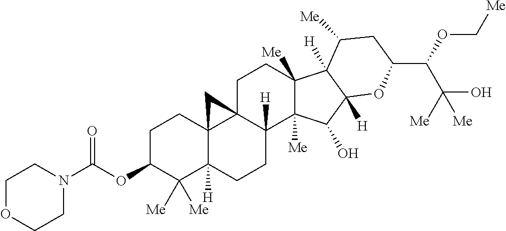 | 343 |

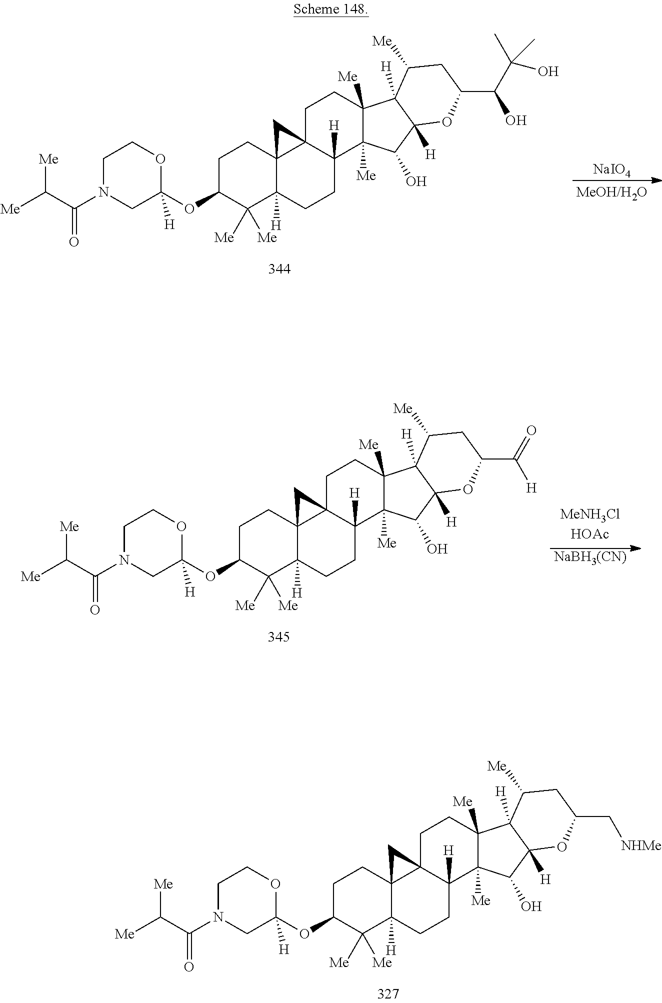

Procedure:

To a solution of compound 344 (0.26 mmol, 169 mg) in MeOH (3.4 mL) and H₂O (1.7 mL) was added NaIO₄ (1.05 mmol, 224 mg). The mixture was stirred at rt for 17 h, quenched with 1.0 M HCl (3 mL), and extracted with CH₂Cl₂. The organic layers were combined, washed with 10% NaOAc, and dried over Na₂SO₄. Removal of the solvent in vacuo provided compound 345 (145 mg) which was used for next step.

Procedure:

To a solution of 345 (0.084 mmol, 49 mg) in MeOH (0.4 mL) and CH₂Cl₂ (0.4 mL) was added methylamine hydrochloride (0.168 mmol, 11 mg), acetic acid (0.168 mmol, 10 μL), and a 1.0 M solution of NaBH₃(CN) in THF (0.084 mmol, 84 μL). The reaction was stirred at rt for 3.5 h and then quenched with sat. NaHCO₃. The mixture was extracted with CH₂Cl₂, dried over Na₂SO₄, and concentrated in vacuo. Purification of the residue by silica gel chromatography with 10% MeOH/CH₂Cl₂ (with 1% Et₃N) provided compound 327 [39 mg, m/z=601.7 (M+H⁺)].

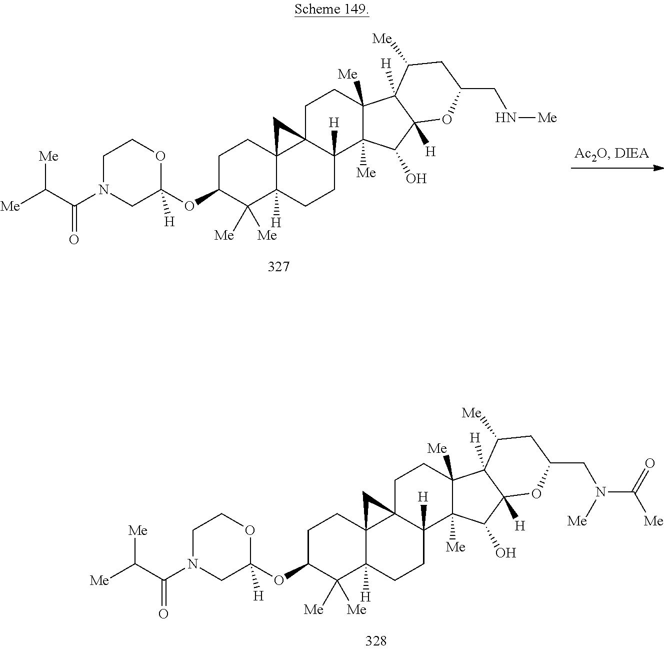

Procedure:

To a solution of compound 327 (0.011 mmol, 6.5 mg) in CH₂Cl₂ (0.5 mL) was added N,N-diisopropylethylamine (0.096 mmol, 16.8 μL) and acetic anhydride (0.011 mmol, 1.1 mg). The resulting solution was stirred at rt for 1 h, quenched with 5% NaHCO₃, extracted with CH₂Cl₂, dried over Na₂SO₄, and concentrated in vacuo. Purification of the residue by silica gel chromatography with 5% MeOH/CH₂Cl₂ provided compound 328 [3.2 mg, m/z=643.7 (M+H⁺)].

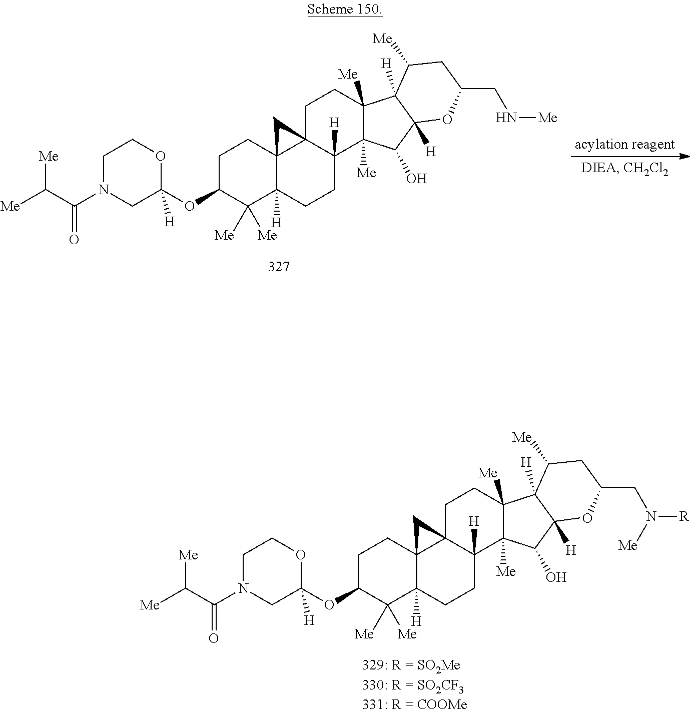

Procedure:

General procedure for preparation of compounds 329, 330, and 331: To a solution of compound 327 (0.025 mmol, 15 mg) in CH₂Cl₂ (0.5 mL) was added N,N-diisopropylethylamine (0.075 mmol, 13 μL) and an acylating agent (0.025 mmol). The resulting solution was stirred at rt for 1 h, quenched with MeOH, and concentrated in vacuo. Purification of the residue by C18 column chromatography (10-100% MeCN/H₂O with 0.1% HCO₂H) provided the desired products. Compound 329 [m/z=679.7 (M+H⁺)]; Compound 330 [m/z=733.4 (M+H⁺)]; Compound 331 [m/z=659.7 (M+H⁺)].

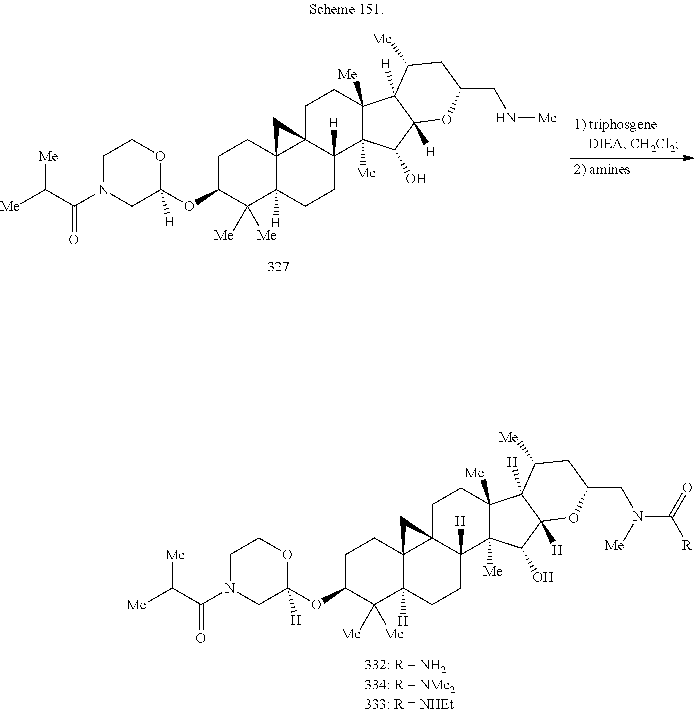

Procedure:

To a solution of compound 327 (0.018 mmol, 11 mg) in CH₂Cl₂ (0.36 mL) was added N,N-diisopropylethylamine (0.036 mmol, 6.3 μL) and triphosgene (0.018 mmol, 5.3 mg). The resulting solution was stirred at rt for 30 min followed by addition of 0.5 mL of an amine (NH₄OH, NHMe₂, or EtNH₂) The mixture was stirred at rt for 15 min and concentrated in vacuo. Purification of the residue by C18 column chromatography (40-100% MeCN/H₂O with 0.1% HCO₂H) provided the desired products. Compound 332 [m/z=644.4 (M+H⁺)]; Compound 334 [m/z=672.5 (M+H⁺)]; Compound 333 [m/z=672.7 (M+H⁺)]

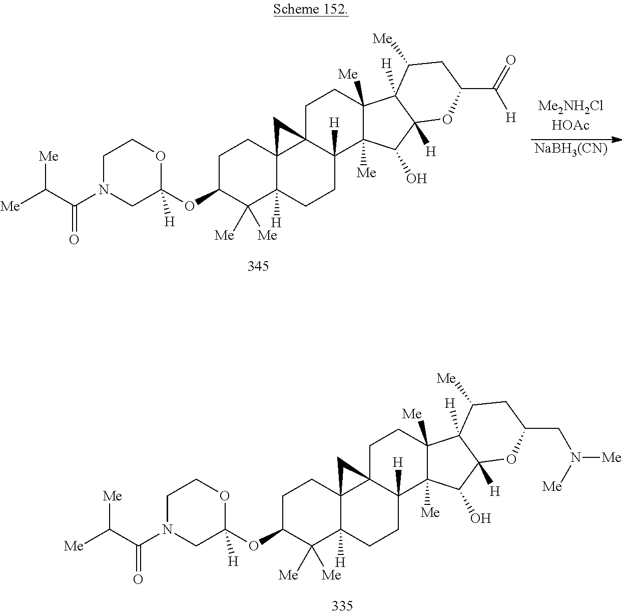

Procedure:

To a solution of compound 345 (0.038 mmol, 22 mg) in MeOH (0.25 mL) and CH₂Cl₂ (0.25 mL) was added dimethylamine hydrochloride (0.056 mmol, 4.6 mg), acetic acid (0.11 mmol, 6.4 μL), and a 1.0 M solution of NaBH₃(CN) in THF (0.026 mmol, 26 μL). The reaction was stirred at rt for 2.5 h and then quenched with sat. NaHCO₃. The mixture was extracted with CH₂Cl₂, dried over Na₂SO₄, and concentrated in vacuo. Purification of the residue by silica gel chromatography with 10-15% MeOH/CH₂Cl₂ (with 1% Et₃N) provided compound 335 [8.2 mg, m/z=615.5 (M+H⁺)].

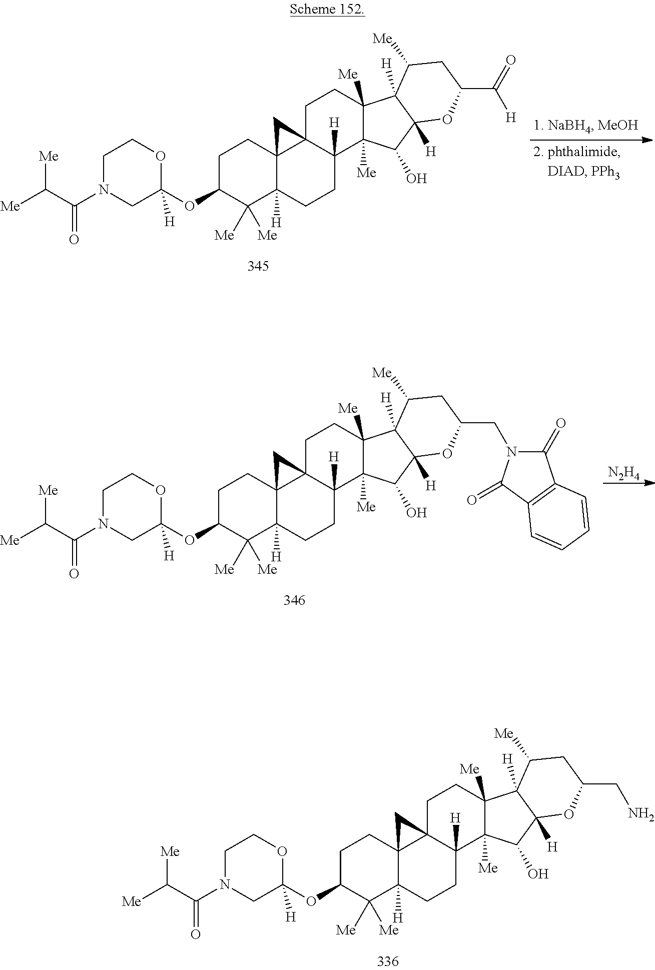

Procedure:

To a solution of compound 345 (0.064 mmol, 37.4 mg) in MeOH (1.0 mL) was added NaBH₄ (0.064 mmol, 2.4 mg). The reaction was stirred at rt for 0.5 h and then quenched with H₂O. The mixture was extracted with CH₂Cl₂ and dried over Na₂SO₄. Removal of the solvent in vacuo provided the alcohol product which was used for the next step without purification.

The alcohol prepared as above (0.034 mmol, 20 mg) was dissolved in CH₂Cl₂ (0.4 mL). Triphenylphosphine (0.085 mmol, 22 mg), phthalimide (0.051 mmol, 7.5 mg) and diisopropyl azodicarboxylate (0.085 mmol, 17 μL) were added in order. The reaction solution was stirred at 25° C. for 4.5 h and quenched with MeOH. Removal of the solvent in vacuo and purification of the residue by silica gel chromatography with 20-50% EtOAc/hexane provided compound 346 (23 mg).

Compound 346 (0.032 mmol, 23 mg) was dissolved THF (0.32 mL). A 1.0 M solution of hydrazine (0.064 mmol, 64 μL) was added. The solution was stirred at rt for 15 h. To the mixture was added 1M HCl (128 μL) and the resulting solution was stirred at rt for 3 h. The reaction was quenched with sat. NaHCO₃, extracted with CH₂Cl₂, dried over Na₂SO₄, and concentrated in vacuo. Purification of the residue by silica gel chromatography with 15-20% MeOH/CH₂Cl₂ (with 1% Et₃N) provided compound 336 [9 mg, m/z=587.5 (M+H⁺)].

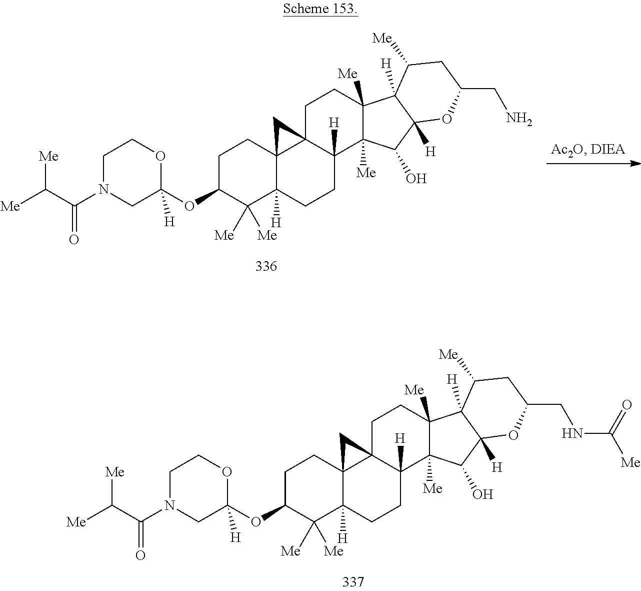

Compound 337 [m/z=629.5 (M+H⁺)] was prepared using the same protocol described in Scheme 149.

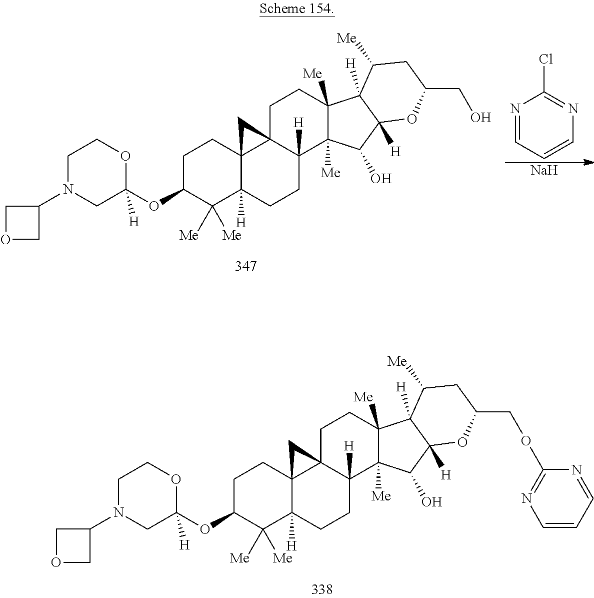

Procedure:

Compound 347 (0.0075 mmol, 4.3 mg) was dissolved in DMF (0.2 mL) and CH₂Cl₂ (0.1 mL). NaH (60%, 0.015 mmol, 0.6 mg) was added. The mixture was stirred for 5 min followed by addition of 2-chloropyrimidine (0.0075 mmol, 0.9 mg). The resulting solution was stirred at rt for 15 h and quenched with MeOH. Removal of the solvent in vacuo and purification of the residue by C18 column chromatography (40-100% MeCN/H₂O with 0.1% HCO₂H) provided compound 338 [m/z=652.4 (M+H⁺)].

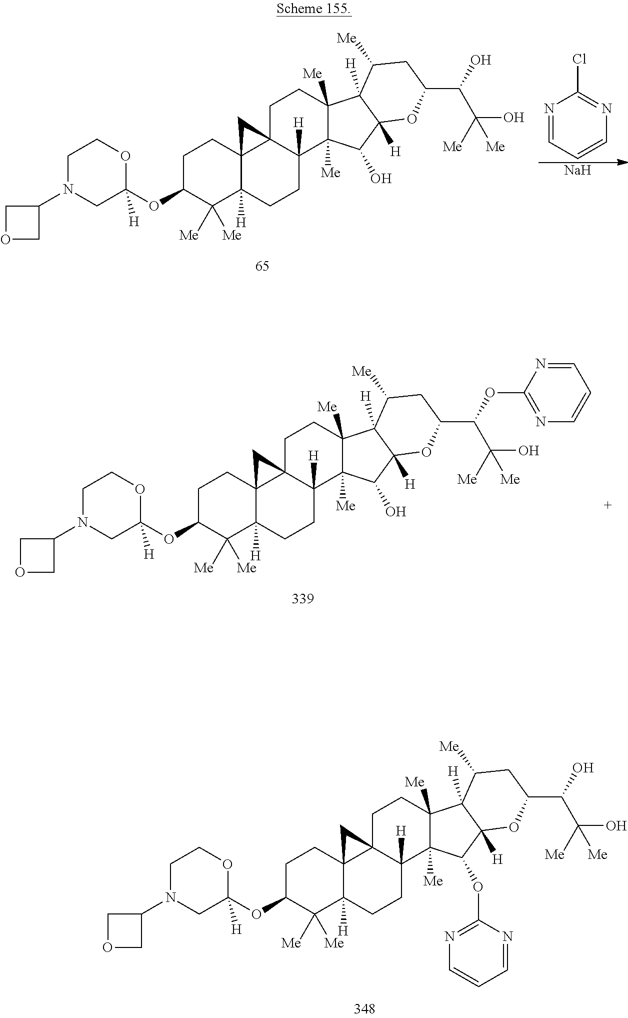

Compounds 339 [m/z=710.5 (M+H⁺)] and 348 [m/z=710.5 (M+H⁺)] were prepared from 65 employing the same protocol described in Scheme 154.

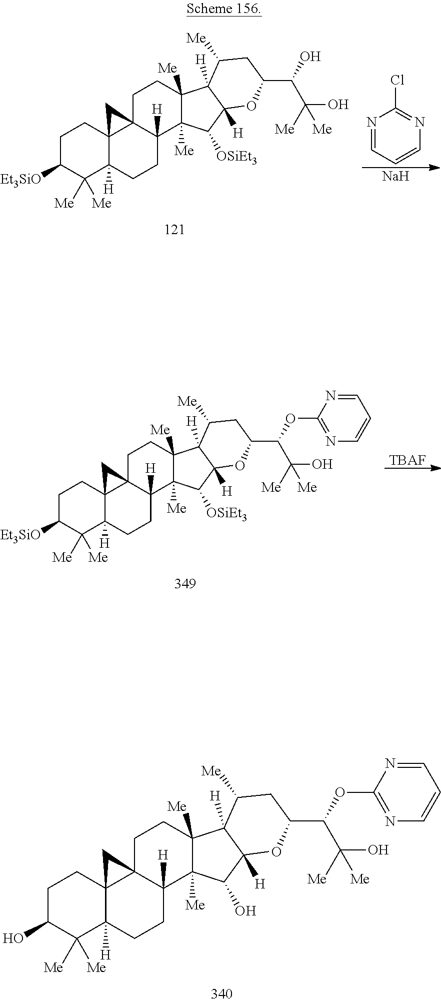

Procedure:

To a solution of compound 121 (0.03 mmol, 22 mg) in THF (0.5 mL) at 0° C. was added NaH (60%, 0.066 mmol, 2.7 mg). The mixture was stirred at 0° C. for 5 min followed by addition of 2-chloropyrimidine (0.033 mmol, 3.8 mg). The resulting solution was stirred at rt for 20 h, quenched with MeOH, and concentrated in vacuo. Purification of the residue by silica gel chromatography with 20-30% EtOAc/hexane provided compound 349 (8.6 mg).

Procedure:

Compound 349 (0.011 mmol, 8.6 mg) was dissolved in THF (0.5 mL). Tetrabutylammonium fluoride (1.0 M/THF, 0.033 mmol) was added. The resulting solution was stirred at rt for 1.5 h and quenched with water. The mixture was extracted with CH₂Cl₂, dried over Na₂SO₄, and concentrated in vacuo. Purification of the residue by silica gel chromatography with 5% EtOH/EtOAc provided compound 340 [m/z=569.4 (M+H⁺)].

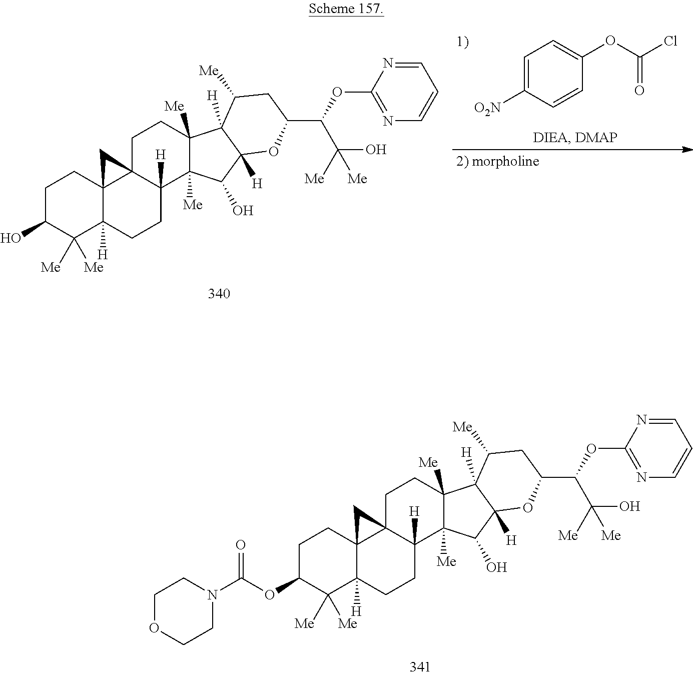

Procedure:

To a solution of compound 340 (0.011 mmol, 6 mg) in CH₂Cl₂ (0.3 mL) was added diisopropylethylamine (0.033 mmol, 5.7 μL), 4-nitrophenyl chloroformate (0.022 mmol, 4 mg), and 4-dimethylaminopyridine (0.011 mmol, 1.3 mg). The resulting solution was stirred at rt for 3 h and then quenched with morpholine (20 μL). Removal of the solvent in vacuo and purification of the residue by C18 column chromatography (10-100% MeCN/H₂O with 0.1% HCO₂H) provided compound 341 [m/z=682.5 (M+H⁺)].

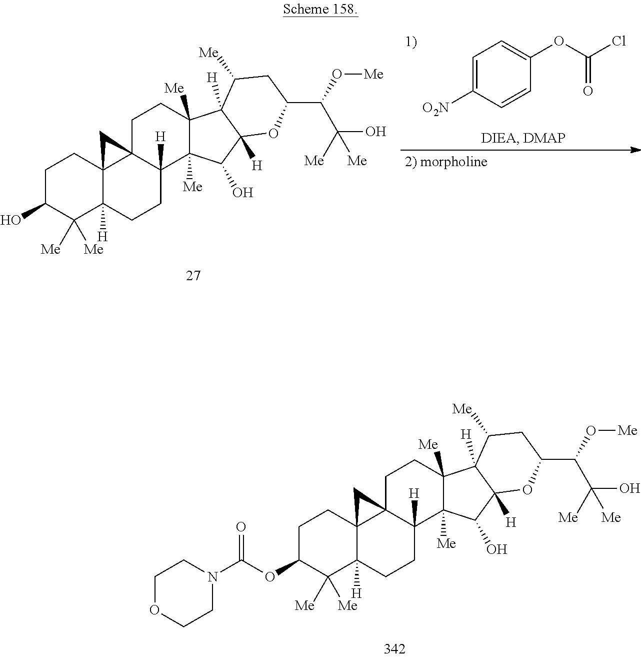

Procedure:

Compound 342 [m/z=640.5 (M+Na⁺)] was prepared from 27 employing the same protocol described in Scheme XX.

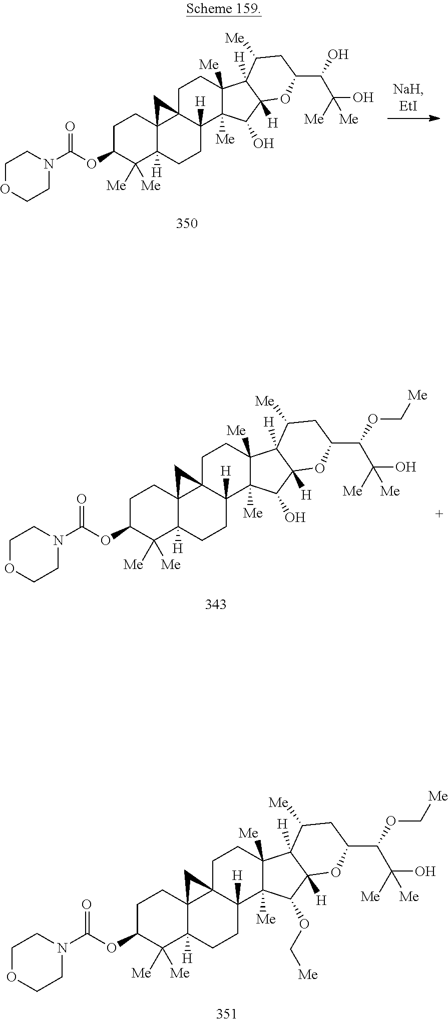

Procedure:

To a solution of compound 350 (0.023 mmol, 14 mg) in THF (0.5 mL) was added NaH (60%, 0.093 mmol, 3.7 mg). The mixture was stirred at rt for 5 min followed by addition of EtI (0.035 mmol, 2.8 μL). The resulting solution was stirred at 40° C. for 15 h, quenched with MeOH, and concentrated in vacuo. The crude product was taken up in DMSO/MeCN (3/1) and filtered. Purification of the filtrate by C18 column chromatography (50-90% MeCN/H₂O with 0.1% HCO₂H) provided compound 343 [m/z=654.5 (M+NO] and 351 [m/z=682.5 (M+Na⁺)]

TABLE 36
Compounds

| | |
|---|---|
| 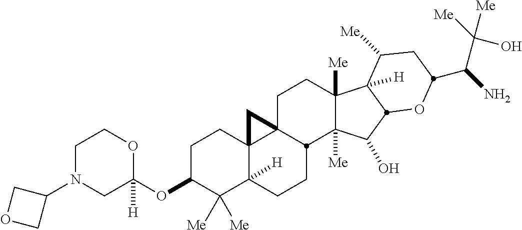 | 352 |
| 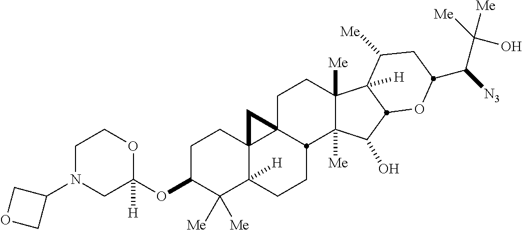 | 353 |
| 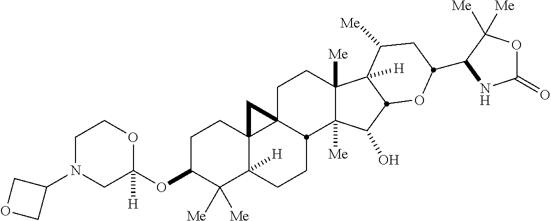 | 354 |
| 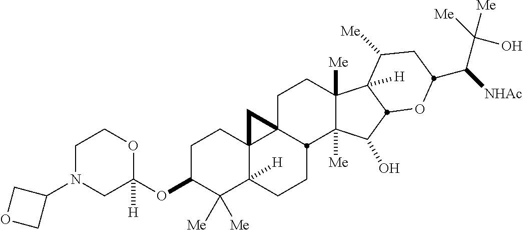 | 355 |
| 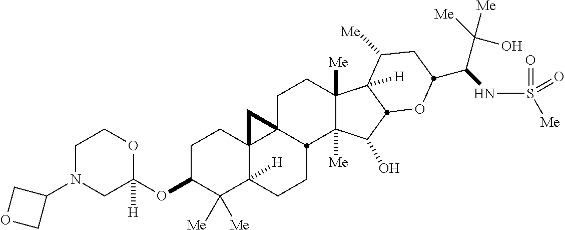 | 356 |
| 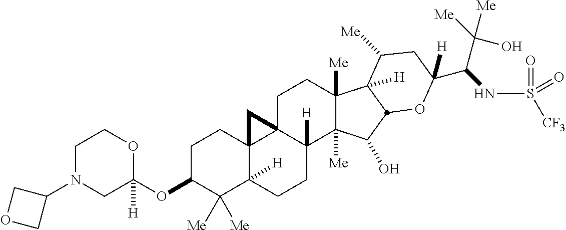 | 357 |

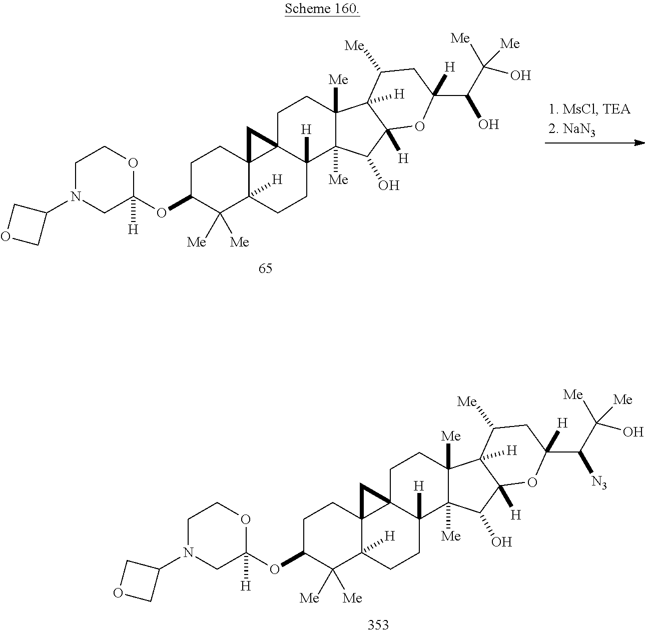

Step 1:

Methanesulfonyl chloride (41.3 uL, 0.532 mmol) was added to diol 65 (280 mg, 0.443 mmol) and Et₃N (308 uL, 2.22 mmol) in DCM (5 mL). Three additional 10 uL portions MsCl were added followed by a 5 uL portion and LC/MS showed 67% M+1=710 (desired product), 17% M+1=632 (starting material) and 16% M+1=788 (bis mesylation). Another peak M/Z=678 was observed. The solution was partitioned between DCM and water, the organic layer dried, and the solvent removed to give an oil that was used without further purification.

Step 2: Sodium azide (200 mg) was added to the mesylate from the previous step and the solution was heated at 140° C. overnight. LC/MS shows major peak m/z=657.4, this mass is consistent with the azide (M+1). m/z=614.4 was also observed as a minor peak, this mass is consistent with an intermediate epoxide (M+1). This peak was the major peak at the 1 h time point. The crude product was purified by biotage chromatography (10 g column, 40-100% EA/Hex) to give 140 mg of azide 353.

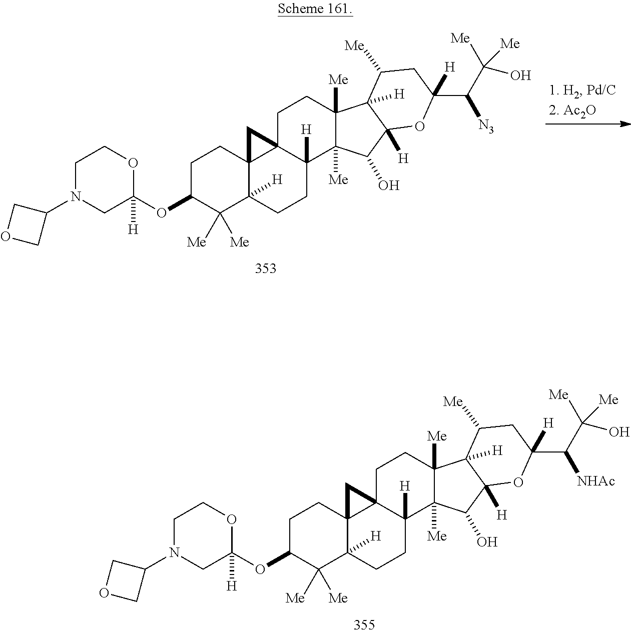

Step 1:

Pd/C (20 mg) was added to the azide 353 (20 mg) in MeOH under N₂. The solution was then purged by bubbling H₂ throught the solution with a needle attached to a balloon. The needle was then raised above the solvent level and the mixture was stirred vigouously overnight. LC/MS shows complete conversion of M+1=578 to M+1=631, consistent with amine 352. The reaction product was used crude without further purification in amide formation.

Step 2:

Ac₂O (3.1 uL) was added to the amine 352 (19 mg) and Et₃N (12.5 uL) in 1 mL DCM. LC/MS after 5 minutes showed good conversion to the acetamide (M+1). Purified by reverse phase HPLC (10-100% ACN/H₂O) to give 14 mg amide 355.

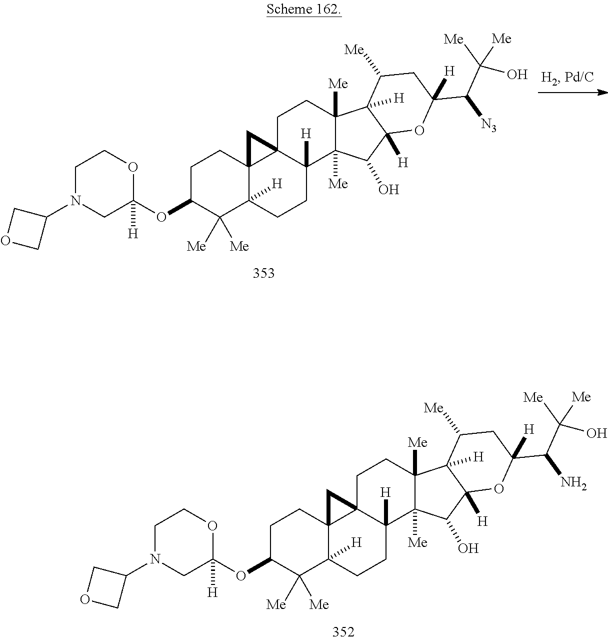

Procedure:

Pd/C (40 mg) was added to the azide 353 (75 mg) in MeOH under N₂. The solution was then purged by bubbling H₂ throught the solution with a needle attached to a balloon. The needle was then raised above the solvent level and the mixture was stirred vigouously overnight. LC/MS shows complete conversion of M+1=578 to M+1=631. Amine 352 was submitted to the assay without further purification.

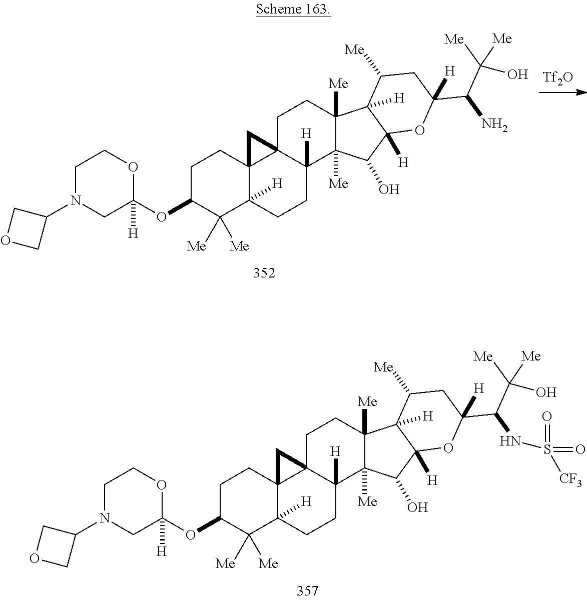

Procedure:

Tf₂O solution (33 uL, 1M in DCM) was added to amine 352 (19 mg) and Hunig's base (10.7 uL) in DCM (1 mL) at rt under N₂. After 5 min LC/MS indicated good conversion to the triflamide. The solvent was removed and the residue was purified by reverse phase HPLC to give 5 mg trifilamide 357.

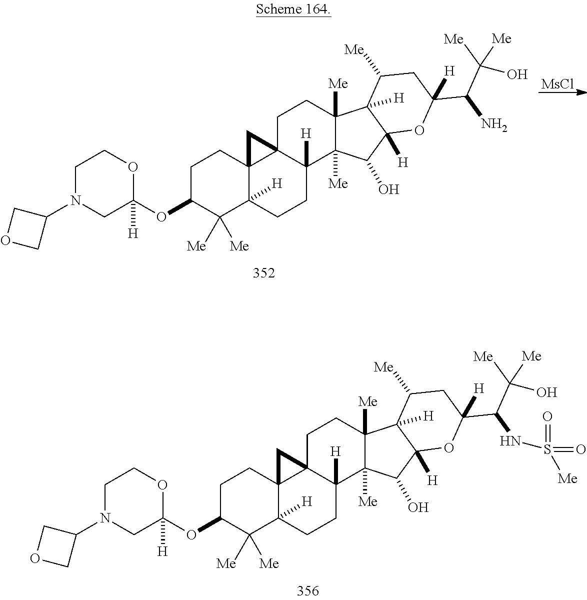

Procedure:

Mesyl chloride (2.8 uL) was added to the amine (19 mg) and Hunig's base (10.8 uL) in DCM (1 mL) at rt under N₂. After 5 min LC/MS indicated good conversion to the sulfonamide. The solvent was removed and the residue was purified by reverse phase HPLC to give 2.5 mg sulfonamide 356.

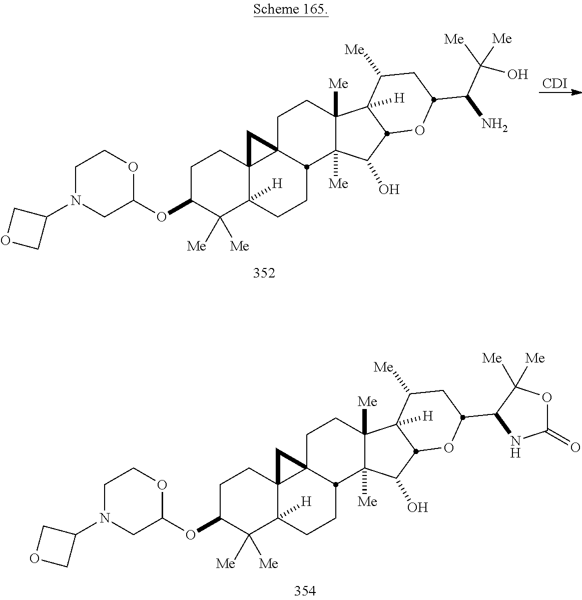

Procedure:

Carbonyldiimidazole (5.1 mg) was added to amino alcohol 352 (20 mg) in DCM (1 mL) and stirred for 1 h. LC/MS shows complete conversion of M+1=631 to M+1=657. Purified by C18 HPLC 10-100% ACN/H₂O to give 3.9 mg cyclic carbamate 354.

TABLE 37
Compounds

| | |
|---|---|
| 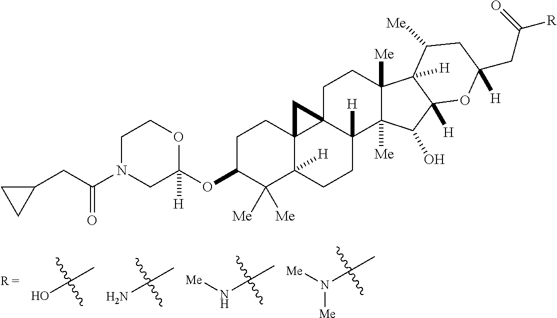 | E-67 |

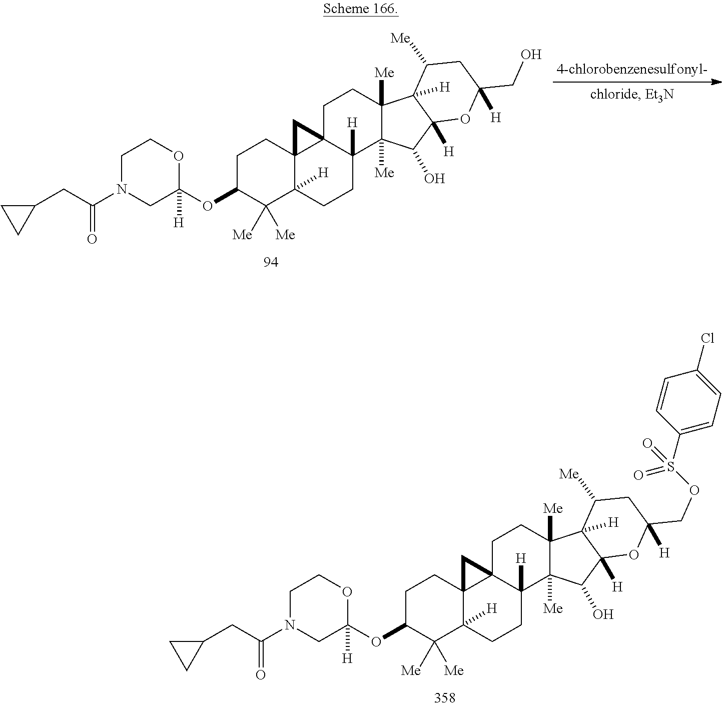

Procedure:

Sulfonyl chloride (633 mg, 3.00 mmol) was added to triethylamine (560 uL, 4.00 mmol) and diol 94 (293 mg, 0.488 mmol) in DCM (1 mL), and allowed to stir for 1 h. LC/MS and TLC indicates no remaining starting material, TLC shows one less polar spot has formed. 500 uL N,N-dimethylethanolamine was added to quench the sulfonyl chloride and the mixture was partitioned between DCM and 1 M KH₂SO₄. Organic layer was dried and concentrated and then purified by biotage (20-100% EA/Hex), 25 g column to give sulfonate 358 (386 mg). LC/MS shows M+1 peak, NMR consistent with product.

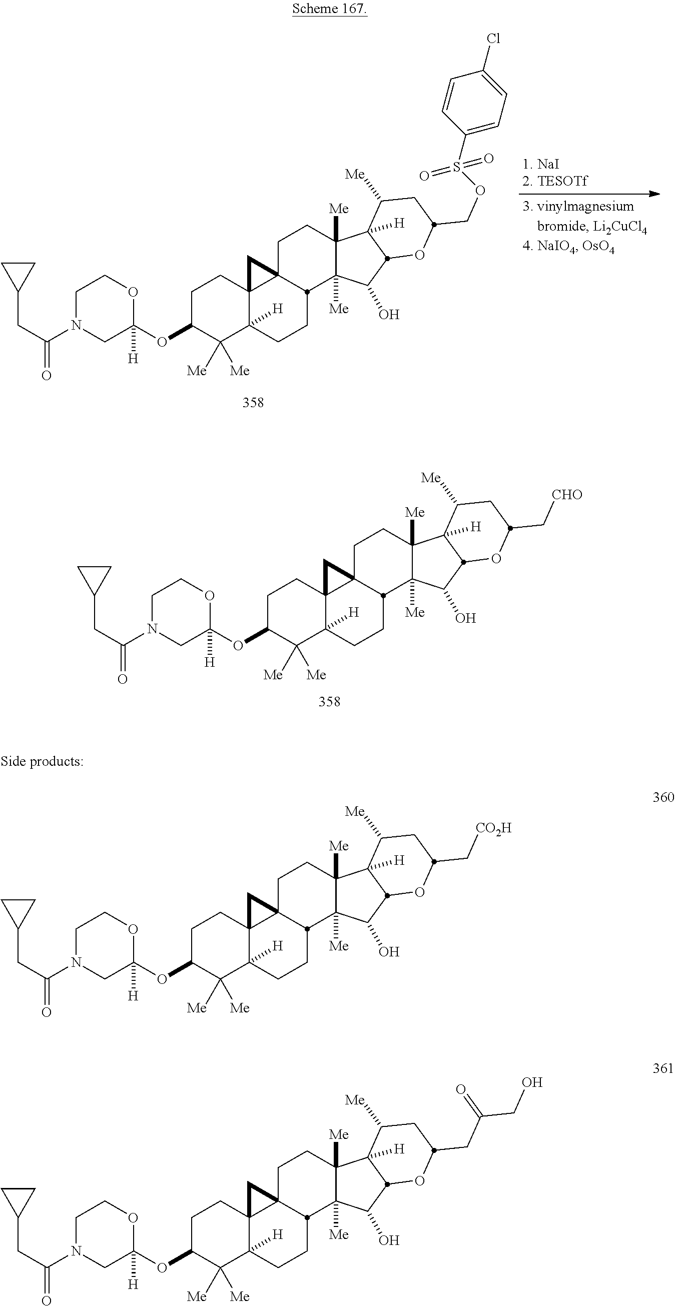

Step 1:

NaI (600 mg, 4.00 mmol), NaHCO₃ (42 mg, 0.50 mmol) and sodium sulfite (63 mg, 0.50 mmol) were added to sulfonate 358 (386 mg, 0.499 mmol) in MEK (2.0 mL) then heated in a closed vessel for 30 min at 90° C. plate temperature. Partition between DCM and 1 M Na₂SO₃, dry organic Na₂SO₄. Solvent was removed under reduced pressure. LC/MS shows M+1, TLC similar R_{f} to starting material, NMR is consistent with product. Use without further purification.

Step 2:

Triethylsilyltrifluoromethanesulfonate (225 uL, 0.998 mmol) was added to crude iodide from previous step and 2,6-lutidine (290 uL, 2.50 mmol) in dry DCM (5 ml). TLC showed complete conversion to a less polar spot on TLC. Solution purified by biotage chromatography 25-100% EA/Hex, 25 g column. Solvent was removed and residue was used in the next step.

Step 3:

Li₂CuCl₄ was added to iodide in 400 uL THF, dissolved, then cooled to −78° C. Vinylmagnesium bromide was added and the solution was allowed to stir for 1 h. TLC indicated no change (30% EA/Hex), previous experience has showed that starting material and product have the same polarity. Saturated NH₄Cl was added and the mixture was allowed to warm to room temperature. Partitioned between MBTE and water and the organic layer was washed with brine. The solution was dried over Na₂SO₄ then concentrated. Purified 4-40% EA/Hex chromatography to give 330 mg. NMR shows 75% conversion.

Step 4:

HCl (1 mL, 1N) was added to the alkene in methanol to remove the TES ether. The solution was partitioned between water and DCM, dried (Na₂SO₄) and concentrated. OsO₄ (5.8 mg, 231 uL 2.5% solution in t-BuOH) was added to the alkene in THF (9 mL) and water (3 mL) followed by NaIO₄ (488 mg, 2.28 mmol). TLC showed formation of a more polar spot. The solution was stirred vigorously overnight then was partitioned between DCM and water. The organic layer was dried and concentrated. LC/MS shows several components in the mixture that were separated by reverse phase HPLC(C18, ACN/water). Major product is M+1=612, consistent with the aldehyde 359. M+1=628 corresponds to the C25 acid 360. M+1=642 corresponds to the ketoalcohol 361.

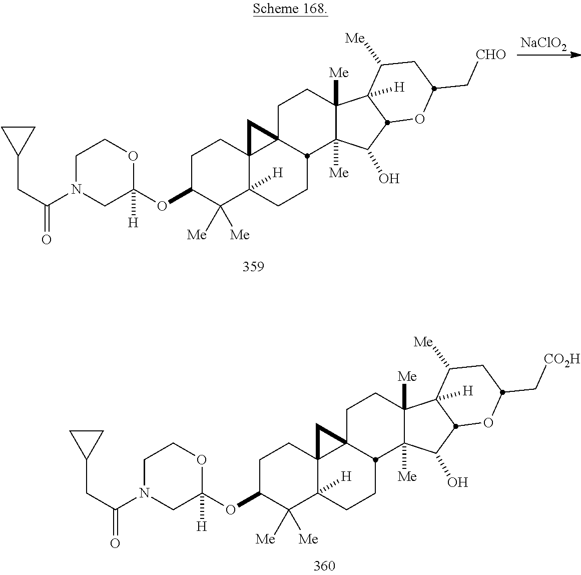

Procedure:

Aldehyde 359 (100 mg) was dissolved in 2-methyl-2-butene solution (2 M in THF, 1.5 mL), t-BuOH (1.5 mL), and H₂O (0.5 mL). Sodium phosphate monobasic (120 mg, 1.00 mmol) was added followed by sodium chlorite (54 mg, 0.597 mmol). The solution was allowed to stir for 3 hours, then partitioned between water and DCM. Wash organic layer with brine, dry Na₂SO₄ and remove solvent. C18 HPLC (10-100% ACN/water gave 39 mg acid 360.

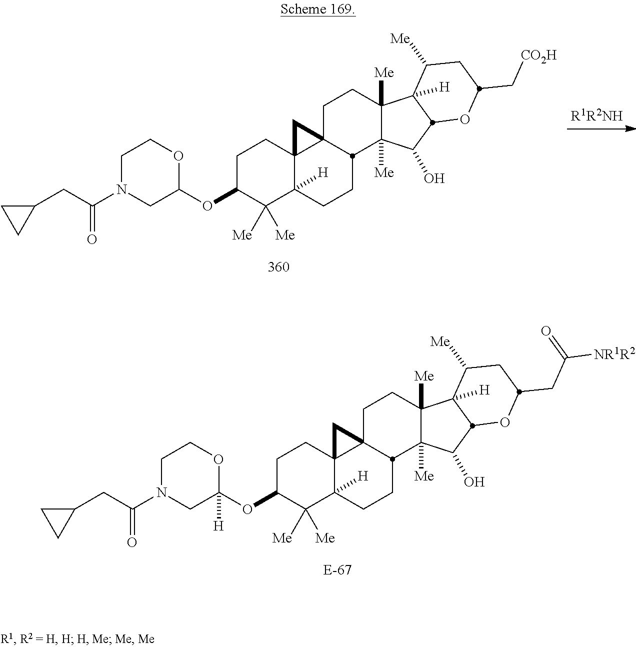

Procedure:

TEA (139 uL, 1.00 mmol), EDC (192 mg, 1.00 mmol), HOBt (153 mg, 1.00 mmol) were added to acid 360 in DMF (1 mL). The solution was then split into three equal parts and 0.33 mmol of either ammonium chloride, methylamine HCl, or dimethylamine HCl were added and the solutions were heated to 100° C. for 30 min. LC/MS shows complete conversion of each to the respective amides. Partition between MBTE/water, wash MBTE with water followed by brine. Dry MgSO₄. Reverse phase HPLC 20-100% ACN/water. Gave approximately 9 mg each product.

TABLE 38
Compounds

| | |
|---|---|
| 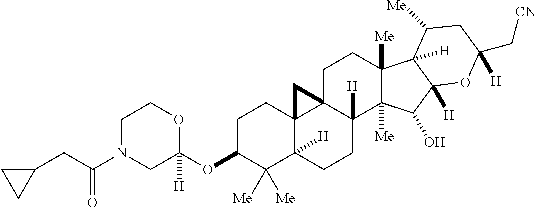 | 361 |
| 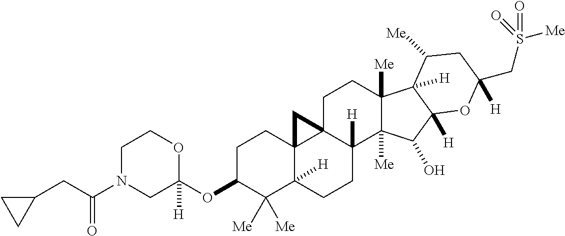 | 362 |
| 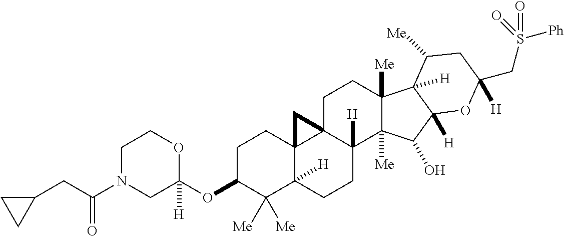 | 363 |

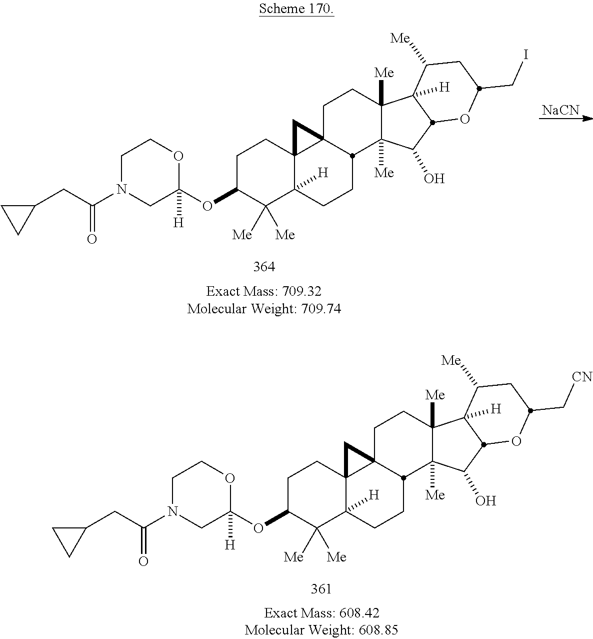

Procedure:

Sodium cyanide (98 mg, 2.00 mmol) was added to iodide (11 mg, 0.015 mmol) in DMF (1 ml) and then heated to 100° C. for 10 minutes. The solution was partitioned between MTBE and water. Organic layer was washed with water then brine. Dry Na₂SO₄ then remove solvent. Reverse phase HPLC (20-100% ACN/water) gave 7 mg cyanide 361.

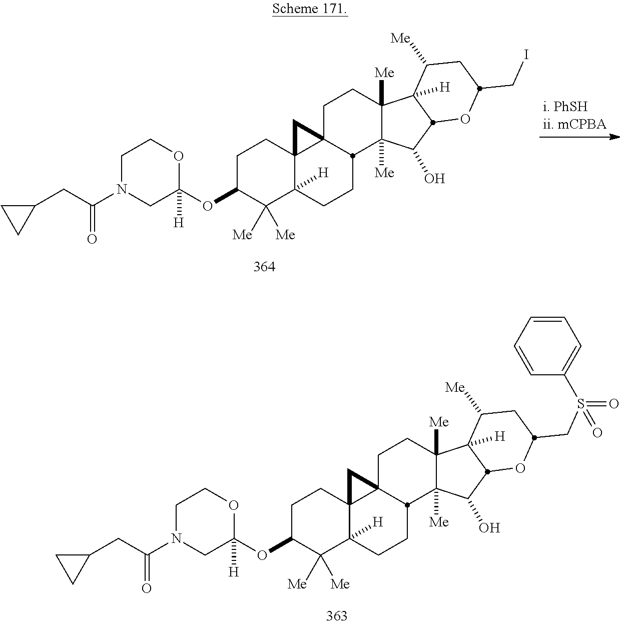

Procedure:

Thiophenol (27.5 mg, 0.250 mmol) was added to iodide 364 (11 mg, 0.015 mmol) in DMF (0.25 mL) and then heated to 100° C. for 10 minutes. The suspension was partitioned between MTBE and water. The organic layer was washed with water and then brine. Dried with Na₂SO₄ then removed solvent. The residue was dissolved in 1 mL DCM and 25 mg mCPBA was added and the solution was allowed to stand for 30 min at rt. Partitioned solution between MTBE and 1 M K₂CO₃. Washed organic layer with water then brine. Dry Na₂SO₄ then remove solvent. Reverse phase HPLC (20-100% ACN/water) gave 7 sulfone 363.

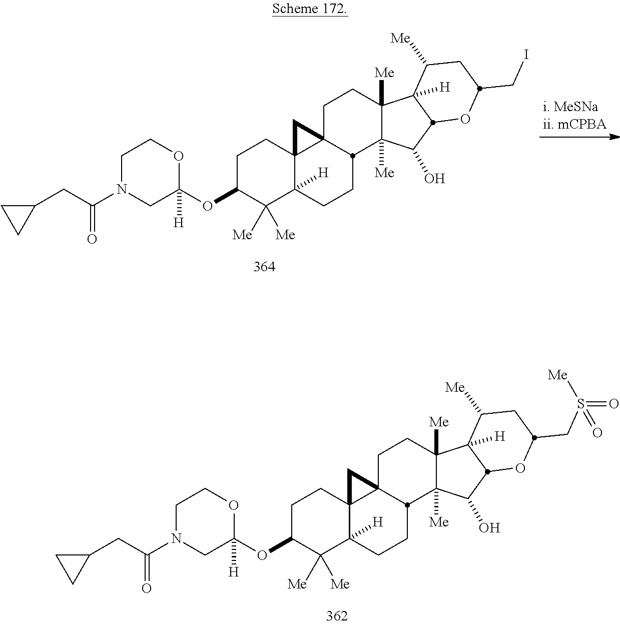

Procedure:

Sodium thiomethoxide (25 mg) was added to the iodide 364 (11 mg, 0.015 mmol) in DMF (0.25 mL) and then heated to 100° C. for 10 minutes. The solution was partitioned between MTBE and water. The organic layer was washed with water then brine, and then it was dried with Na₂SO₄ then the solvent was removed. The residue was dissolved in 1 mL DCM and 25 mg mCPBA was added and the solution was allowed to stand for 30 min at rt. The solution was partitioned between MTBE and 1 M K₂CO₃. The organic layer was washed with water then brine. The solution was dried with Na₂SO₄ then the solvent was removed. Reverse phase HPLC (20-100% ACN/water) gave 7 mg sulfone 362.

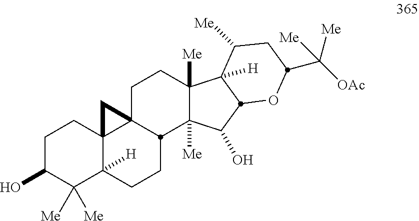

### Example 14

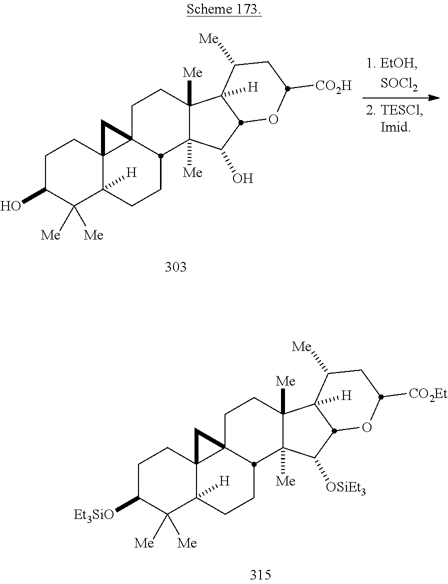

Step 1:

Thionyl chloride (1.69 mL, 23.3 mmol) was added to a solution of acid 303 in 25 mL ethanol. After 1 h, TLC indicated complete conversion to a less polar spot. The solution was partitioned between water and MTBE. The organic layer was washed with sodium bicarbonate (sat'd aq') then water. The organic layer was then dried and concentrated to give the ester that was used without further purification.

Step 2:

TESCl (5.32 g, 31.6 mmol) was added to the diol 303 (5.00 g, 10.5 mmol) and imidazole (4.31 g, 63.3 mmol) In DMF (30 mL) and allowed to stir overnight. The solution was partition between MBTE and water. The organic layer was washed with water then brine and dried over Na₂SO₄. Solvent removed and the residue was purified by chromatography 1-10% EA/hexanes to give 3.0 g ester 315.

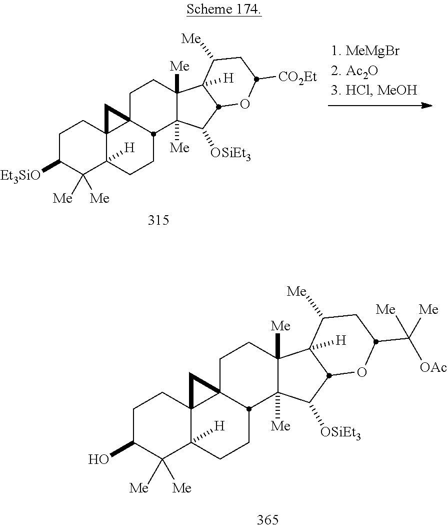

Step 1:

MeMgBr (130 uL, 3.2 M in MeTHF) was added to ester 315 (96 mg, 0.137 mmol) in THF (1 mL) at room temperature. After 15 min TLC (10% EA/Hex) showed some remaining starting material. An additional 130 uL MeMgBr was added, TLC after an additional 30 minutes is below. NH₄Cl was added and the reaction mixture was partitioned between MBTE/water. The organic layer was dried and concentrated to give an oil that was used without further purification.

Step 2:

Acetic anhydride (25.7 uL, 0.273 mmol) was added to the crude product of step 1 (94 mg, 0.136 mmol) in DCM (2 mL) at room temperature. Little reaction was observed after 1 h by TLC. An additional 330 mg DMAP then 250 uL Ac₂O was added and the reaction started to proceed to two less polar spots. The mixture was partitioned between 1 M KHSO₄ and MBTE, washed with Na₂CO₃ then brine, and then dried with Na₂SO₄. The solution was concentrated and purified 2-20% EA hexanes to give a mixture of the two less polar spots. The second contains the desired product and was used without further purification.

Step 3:

The TES-protected diol was dissolved in approximately 5 mL of ethanol and 200 uL 1 N HCl was added. The solvent was removed under reduced pressure. TLC indicated complete conversion of the TES ether spots to a baseline TLC spot. LC/MS shows two major peaks. One spot is consistent with the desired product (M+23=525) and the other consistent with peracetylation in the previous step. RP HPLC gave 9 mg of acetate 365.

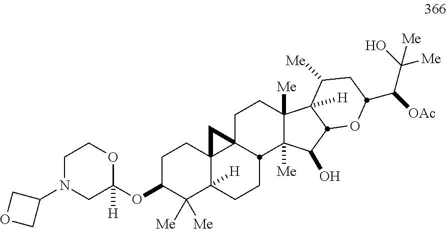

### Example 15

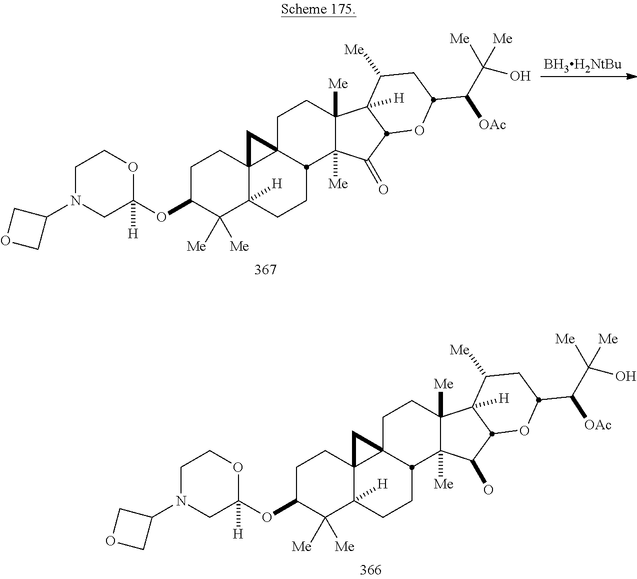

Procedure:

Borane-tButylamine (409 mg, 4.71 mmol) was added to the ketone 367 (2.53 g, 3.77 mmol) in EtOH (15 mL) at room temperature and allowed to stir over the weekend (some gas evolution was observed). LC/MS shows a small amount of remaining ketone. HCl (1 mL of 1N solution was added and the solution was partitioned between 50 mL each CH₂Cl₂ and water. NaOAc (5% w/v, 5 mL) was added and the layers were separated, then the aqueous layer extracted with 50 mL CH₂Cl₂ and the combined organic layers were dried over Na₂SO₄, filtered, and the solvent removed under reduced pressure. Crude NMR shows impurity, and what appears to be C15 isomeric compound (d, 0.7 ppm; reduced integration of peak at 3.72 ppm). Reverse phase biotage (c18) followed by recrystallization twice from MBTE gave >95% pure alcohol major isomer (alcohol down, R), 500 mg. The mother liquors from the recrystallizations were concentrated to give 500 mg 3:1 mixture favoring the alcohol down isomer (R). The mixture was purified by isocratic chromatography (EtOAc over silica, biotage 50 g). (S)-isomer is less polar and purity was enhanced by this first purification. That material was repurified (25 g biotage) to give material that was >90% pure.

TABLE 39
Compounds

| | |
|---|---|
| 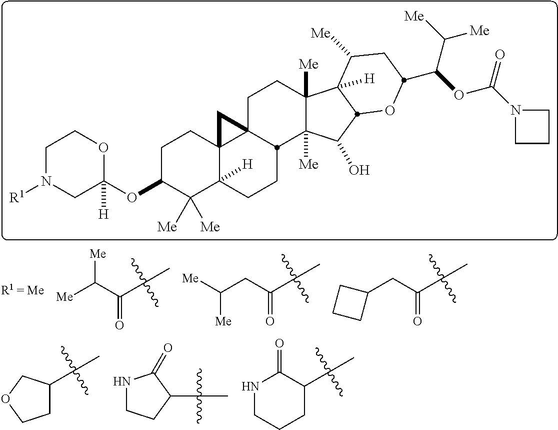 | E-68 |

TABLE 40
Compounds

| | |
|---|---|
| 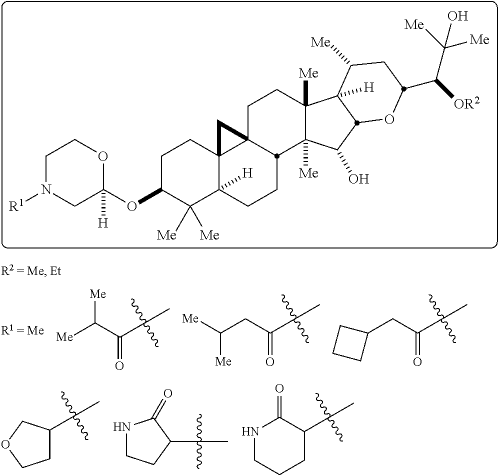 | E-69 |

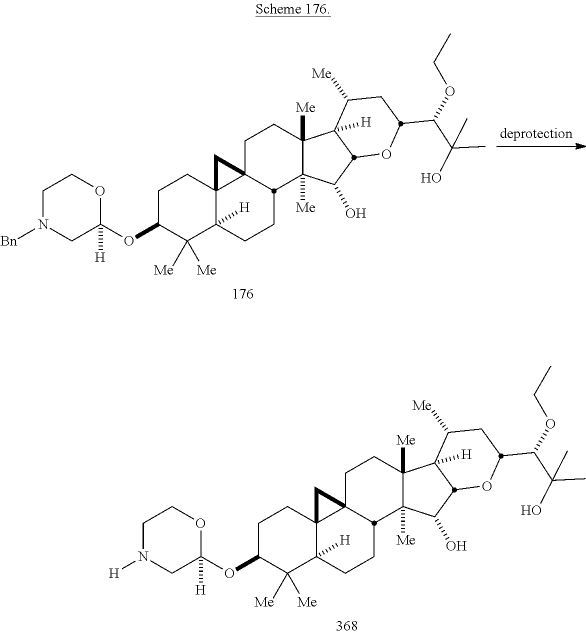

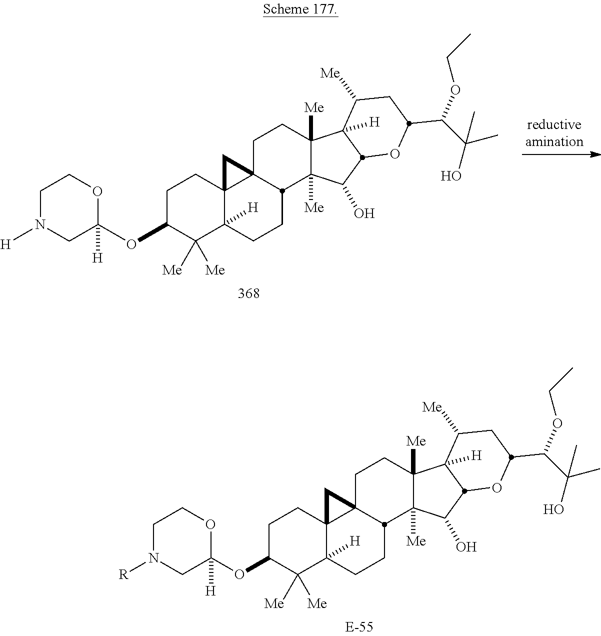

TABLE 41
Compounds
R =

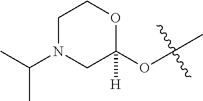 [SMILES: *O[C@H]1CN(C(C)C)CCO1]

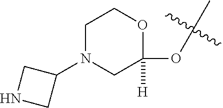 [SMILES: *O[C@H]1CN(C2CNC2)CCO1]

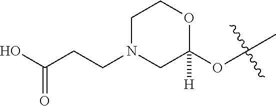 [SMILES: *O[C@H]1CN(CCC(=O)O)CCO1]

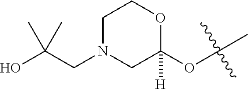 [SMILES: *O[C@H]1CN(CC(C)(C)O)CCO1]

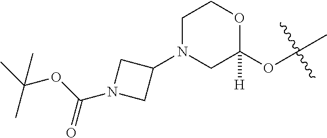 [SMILES: *O[C@H]1CN(C2CN(C(=O)OC(C)(C)C)C2)CCO1]

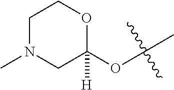 [SMILES: *O[C@H]1CN(C)CCO1]

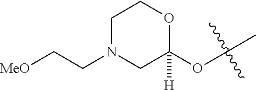 [SMILES: *O[C@H]1CN(CCOC)CCO1]

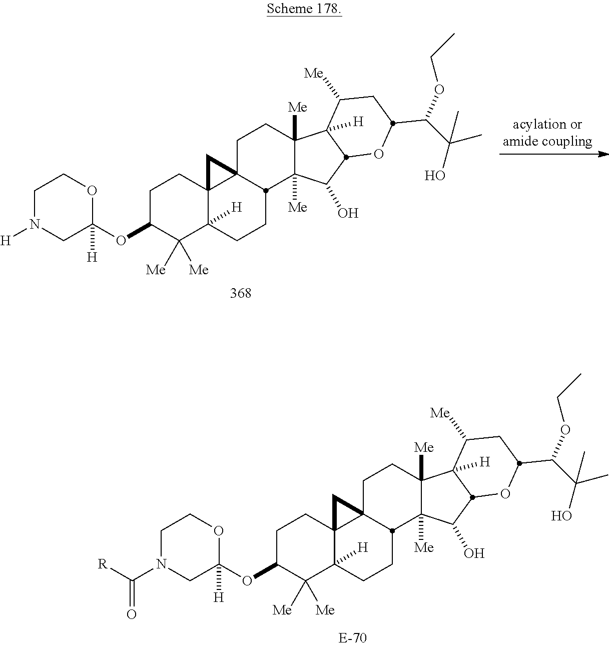

TABLE 42
Compounds
R =

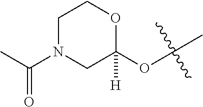 [SMILES: *O[C@H]1CN(C(C)=O)CCO1]

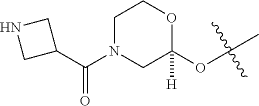 [SMILES: *O[C@H]1CN(C(=O)C2CNC2)CCO1]

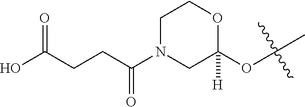 [SMILES: *O[C@H]1CN(C(=O)CCC(=O)O)CCO1]

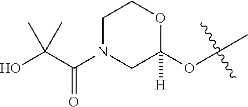 [SMILES: *O[C@H]1CN(C(=O)C(C)(C)O)CCO1]

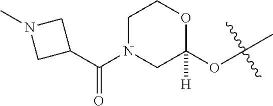 [SMILES: *O[C@H]1CN(C(=O)C2CN(C)C2)CCO1]

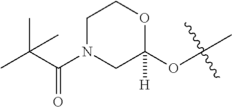 [SMILES: *O[C@H]1CN(C(=O)C(C)(C)C)CCO1]

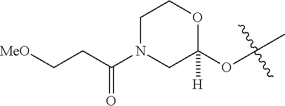 [SMILES: *O[C@H]1CN(C(=O)CCOC)CCO1]

TABLE 43
Compounds

| | |
|---|---|
| 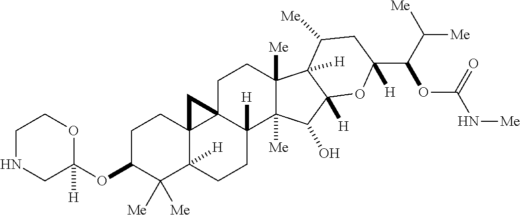 | 369 |
| 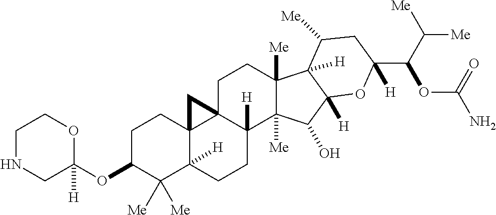 | 370 |
| 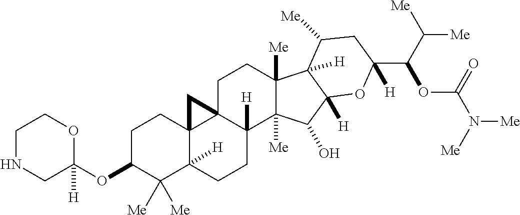 | 371 |
| 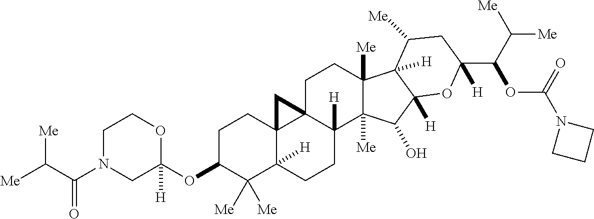 | 372 |
| 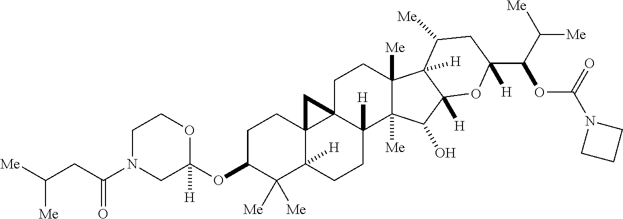 | 373 |

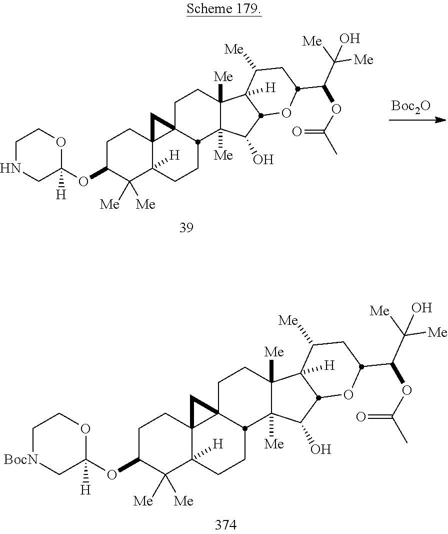

Procedure:

A 50-mL flask was charged with amine 39 (400 mg, 0.65 mmol) and Et₃N (197 mg, 1.95 mmol) in 5 mL of DCM. Then (Boc)₂O (212 mg, 0.97 mmol) was added. The resulting mixture was stirred at room temperature for 2 hours. TLC showed the reaction was completed. Then the mixture was diluted with DCM (50 mL). The organic layer was washed with water (15 mL), brine, dried over Na₂SO₄, filtered and concentrated to give carbamate 374, which was purified by chromatography on silica gel (450 mg, 97%).

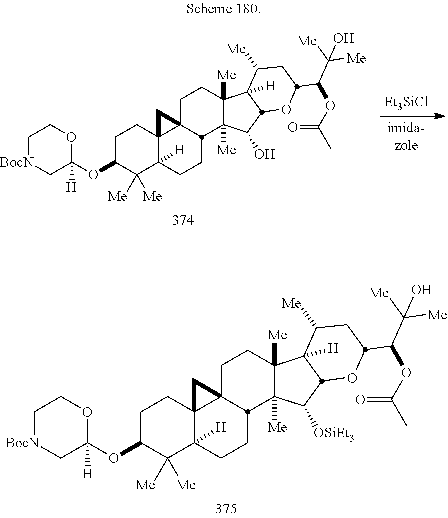

Procedure:

TESCl (150 mg, 1 mmol) was added to alcohol 374 (450 mg, 0.63 mmol) followed by imidazole (214 mg, 3.15 mmol) in DCM (2 mL). TLC showed good balance of conversion after 1 hour. Then the mixture was diluted with DCM (30 mL). The organic layer was washed with water (10 mL×2), brine, dried over Na₂SO₄, filtered and concentrated to give silyl ether 375, which was purified by chromatography (330 mg, 63%).

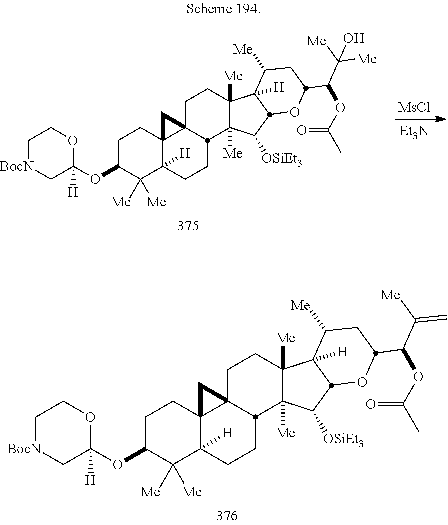

Procedure:

To a solution of alcohol 375 (330 mg, 0.4 mmol) and Et₃N (727 mg, 7.2 mmol) in dry DCM (5 mL) was added MsCl (340 mg, 3 mmol) dropwise slowly in ice water bath. Then the mixture was stirred at room temperature for 1 hour. TLC showed the reaction was completed. Water (30 mL) was added. Then the aqueous layer was extracted with DCM (15 mL×3). The organic layers were combined, washed with brine, dried over Na₂SO₄, filtered and concentrated to give alkene 376 which was purified by chromatography (270 mg, 83%).

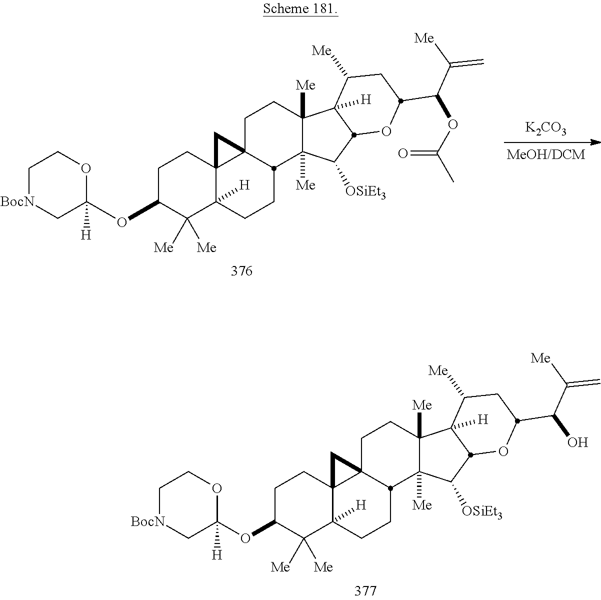

Procedure:

To a solution of acetate 376 (200 mg, 0.25 mmol) in DCM (10 mL) and MeOH (10 mL) was added K₂CO₃ (271 mg, 1.9 mmol). Then the mixture was stirred at room temperature for 14 hours. TLC showed the reaction was completed. The mixture was concentrated to remove MeOH and water (60 mL) was added. The aqueous layer was extracted with DCM (30 mL×3). The organic layers were combined, washed with brine, dried over Na₂SO₄, filtered and concentrated to give alcohol 377 which was used in the next step without purification (170 mg, 88%).

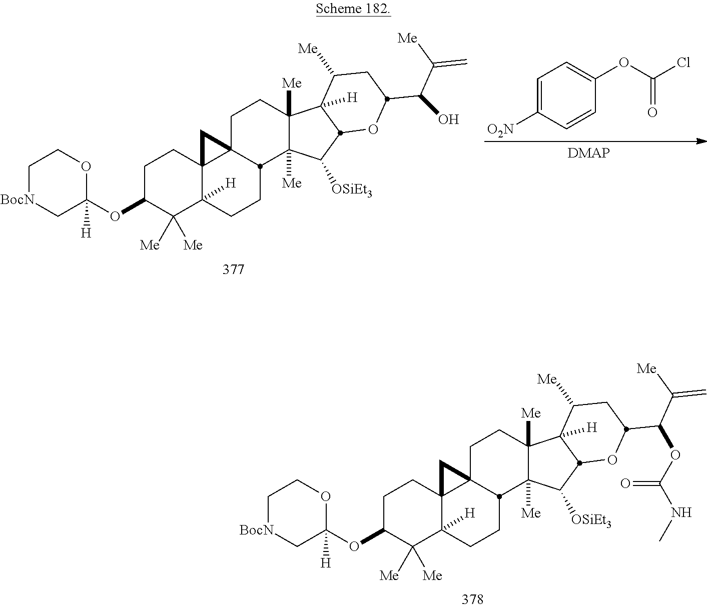

Procedure:

4-Nitrophenyl chloroformate (131 mg, 0.65 mmol) was added to DIEA (67 mg, 0.52 mmol), DMAP (79 mg, 0.65 mmol) and alcohol 377 (50 mg, 0.065 mmol) in dry CH₂Cl₂ (2 mL) under N₂ and allowed to stir for 12 hours. Then NH₂Me (22 mg, 0.33 mmol) was added and the mixture was stirred at rt for another 12 hours. Then the mixture was diluted with DCM (30 mL). The organic layer was washed with water (10 mL×3), brine, dried over Na₂SO₄, filtered and concentrated to give the residue, which was purified by chromatography to provide carbamate 378 (15 mg, 27.8%).

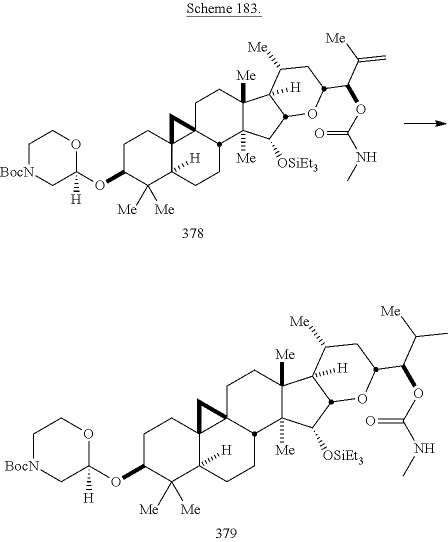

Procedure:

To a solution of alkene 378 (15 mg, 0.018 mmol) in EA (1 mL) and MeOH (5 mL) was added 20% Pd(OH)₂ on carbon (wet) (3 mg) and the flask was fit with a balloon of H₂. The reaction mixture was stirred under an atmosphere of H₂ at rt for 1 hour. The solid was filtered and solvent was removed in vacuo to give compound 379, which was purified by chromatography (10 mg, 67%).

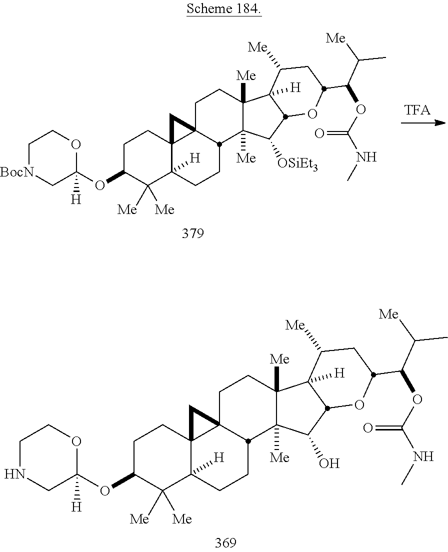

Procedure:

Carbamate 379 (10 mg, 0.012 mmol) was dissolved in TFA/DCM (3 mL) (V/V=20%). Then the mixture was stirred at room temperature for 30 minutes. TLC showed the reaction was completed. Solvent was removed in vacuo to give amine 369 (4.58 mg, 53%). LCMS (m/z): [M+H]⁺ 617

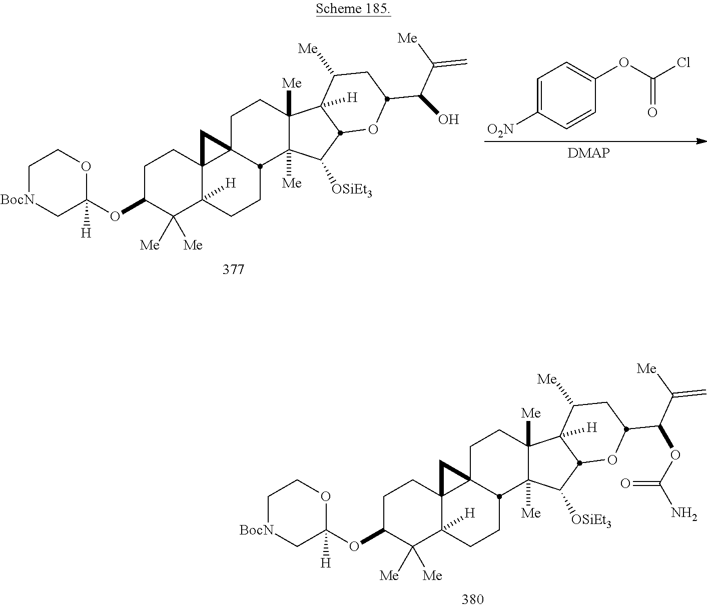

Procedure:

4-Nitrophenyl chloroformate (73 mg, 0.39 mmol) was added to Et₃N (79 mg, 0.78 mmol), DMAP (47 mg, 0.39 mmol) and alcohol 377 (30 mg, 0.039 mmol) in dry CH₂Cl₂ (1 mL) under N₂ and allowed to stir for 12 hours. Then the mixture was stirred at room temperature under NH₃ for another 12 hours. TLC showed the reaction was completed. Water (30 mL) was added. The aqueous layer was extracted with DCM (15 mL×3). The organic layers were combined, washed with brine, dried over Na₂SO₄, filtered and concentrated to give carbonate 380, which was purified by chromatography (20 mg, 63%).

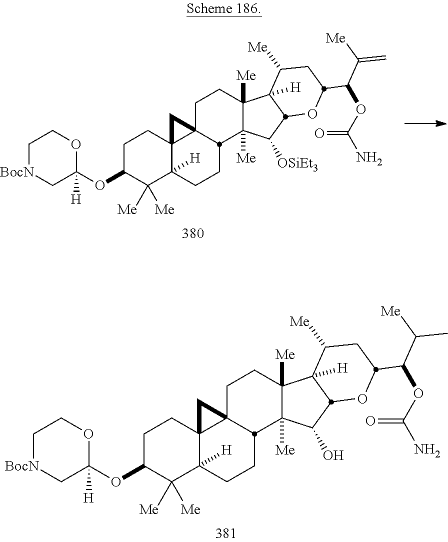

Procedure:

To a solution of carbonate 380 (20 mg, 0.025 mmol) in DCM (1 mL) and MeOH (1 mL) was added PPTS (19 mg, 0.075 mmol,). Then the mixture was stirred at rt for 30 minutes. TLC showed the reaction was completed. Solvent was removed in vacuo to give the crude product which was purified by chromatography (13 mg, 0.0186 mmol). The obtained product was then dissolved in MeOH (3 mL). The mixture was treated with 20% Pd(OH)₂ on carbon (wet) (10 mg) and the flask was fit with a balloon of H₂. The reaction mixture was stirred under an atmosphere of H₂ at rt for 30 min. The solid was filtered and solvent was removed in vacuo to give carbamate 381, which was used for the next step without purification (10 mg, 77%).

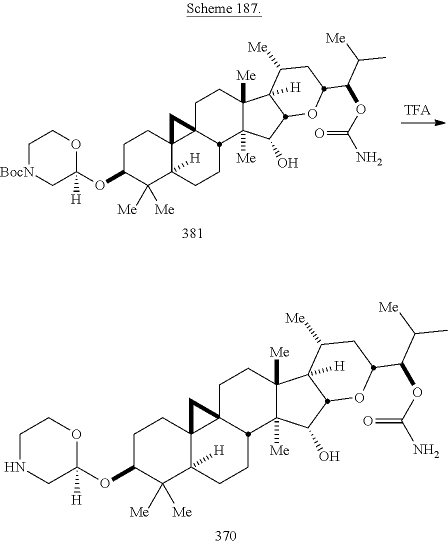

Carbamate 381 (10 mg, 0.014 mmol) was dissolved in TFA/DCM (1 mL) (V/V=20%). Then the mixture was stirred at room temperature for 30 minutes. TLC showed the reaction was completed. Solvent was removed in vacuo to give amine 370 (8.13 mg, 95%). LCMS (m/z): [M+H]⁺ 603

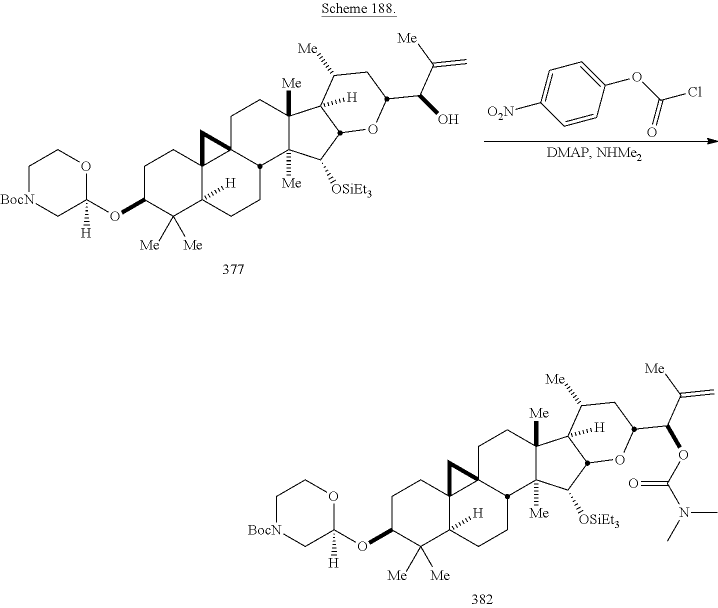

Procedure:

4-Nitrophenyl chloroformate (117 mg, 0.58 mmol) was added to Et₃N (59 mg, 0.58 mmol), DMAP (71 mg, 0.58 mmol) and alcohol 377 (45 mg, 0.058 mmol) in dry CH₂Cl₂ (1 mL) under N₂. After stirring at rt for 12 hours, NHMe₂.HCl (47 mg, 0.58 mmol) was added. Then the mixture was stirred at rt for another 12 hours. TLC showed the reaction was completed. Water (30 mL) was added. The aqueous layer was extracted with DCM (15 mL×3). The organic layers were combined, washed with brine, dried over Na₂SO₄, filtered and concentrated to give carbamate 384 which was purified by chromatography (25 mg, 51%).

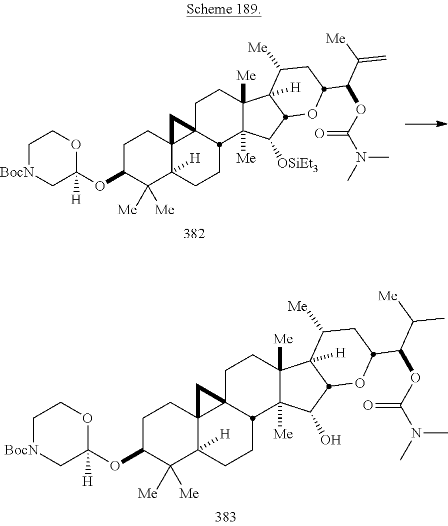

Procedure:

To a solution of alkene 382 (25 mg, 0.03 mmol) in DCM (1 mL) and MeOH (1 mL) was added PPTs (22 mg, 0.09 mmol). Then the mixture was stirred at room temperature for 30 minutes. TLC showed the reaction was completed. Solvent was removed in vacuo to give the crude product which was purified by chromatography (20 mg, 93%). The obtained product was then dissolved in MeOH (3 mL). The mixture was treated with 20% Pd(OH)₂ on carbon (wet) (10 mg) and the flask was fit with a balloon of H₂. The reaction mixture was stirred under an atmosphere of H₂ at rt for 30 min. The solid was filtered and solvent was removed in vacuo to give carbamate 383, which was used for the next step without purification (10 mg, 50%).

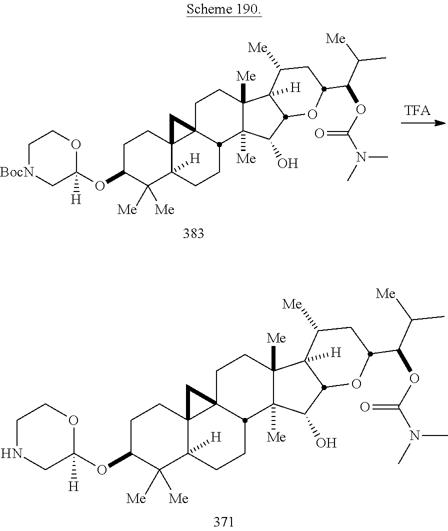

Procedure:

Carbamate 383 (10 mg, 0.014 mmol) was dissolved in TFA/DCM (1 mL) (V/V=20%). Then the mixture was stirred at room temperature for 30 minutes. TLC showed the reaction was completed. Solvent was removed in vacuo to give amine 371, which was purified by preparative HPLC (6.72 mg, 75%). LCMS (m/z): [M+H]⁺ 631.5

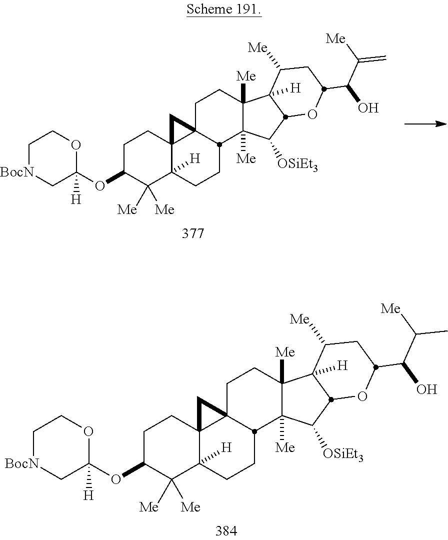

Procedure:

To a solution of alkene 377 (160 mg, 0.2 mmol) in MeOH (10 mL) and EA (2 mL) was treated with 20% Pd(OH)₂ on carbon (wet) (20 mg) and the flask was fit with a balloon of H₂. The reaction mixture was stirred under an atmosphere of H₂ at rt for 30 min. The solid was filtered and solvent was removed in vacuo to give carbamate 384, which was used for the next step without purification (100 mg, 63%).

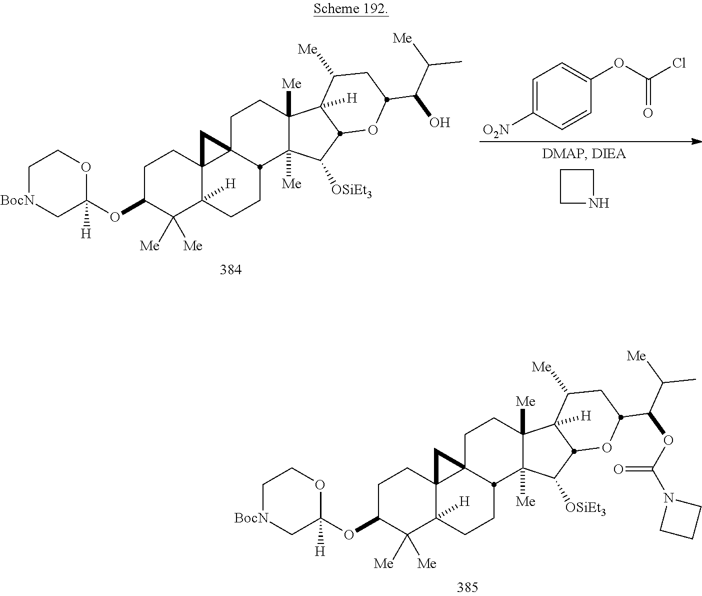

Procedure:

4-Nitrophenyl chloroformate (261 mg, 1.3 mmol) was added to DIEA (252 mg, 1.95 mmol), DMAP (159 mg, 1.3 mmol) and alcohol 384 (100 mg, 0.13 mmol) in dry CH₂Cl₂ (1 mL) under N₂. After stirring at room temperature for 12 hours, azetidine (74 mg, 1.3 mmol) was added. Then the mixture was stirred at room temperature for another 12 hours. TLC showed the reaction was completed. Water (30 mL) was added. The aqueous layer was extracted with DCM (15 mL×3). The organic layers were combined, washed with brine, dried over Na₂SO₄, filtered and concentrated to give carbamate 385, which was purified by chromatography (50 mg, 45%).

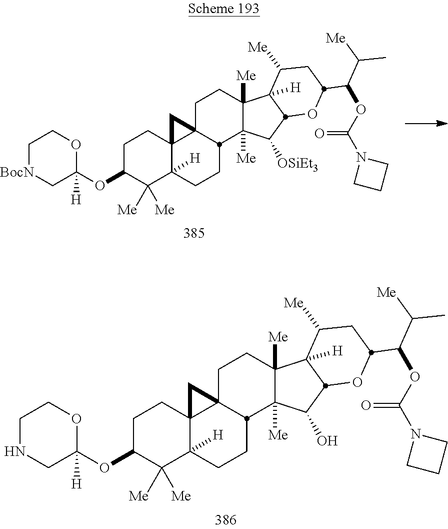

Procedure:

A 50 mL of flask was charged with carbamate 385 (50 mg, 0.058 mmol) dissolved in 20% TFA in DCM (5 mL). The mixture was stirred at room temperature for 30 min. LCMS showed the reaction was finished. Then the mixture was concentrated to give amine 386, which was used for the next step directly (50 mg, crude, 100%)

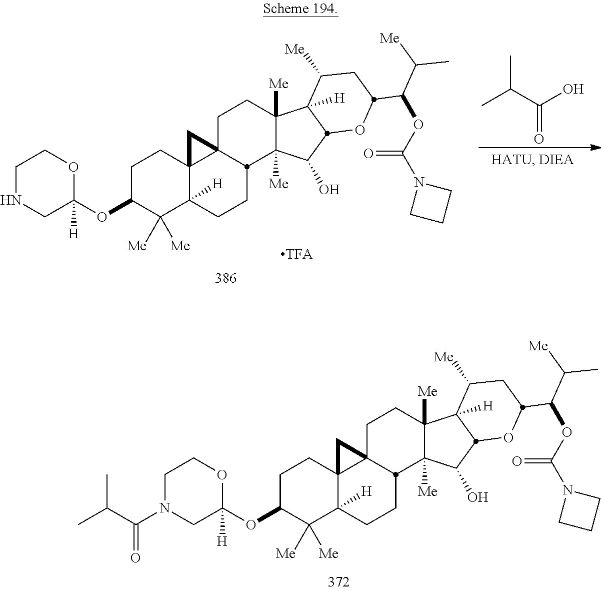

Procedure:

A mixture of amine 386 (10 mg, crude, 14 μmol), isobutyric acid (2 mg, 20 μmol), HATU (8 mg, 20 μmol), DIEA (6 mg, 42 μmol) in CH₂Cl₂ (0.5 mL) was stirred at room rt overnight. The reaction mixture was diluted with CH₂Cl₂ (5 mL) and washed with saturated citri acid (5 mL). The phases were separated and the aqueous phase was extracted with CH₂Cl₂ (2 mL×2). The combined organic phase was dried over Na₂SO₄ and concentrated in vacuo to give a residue which was purified by preparative HPLC to give amide 372 (4.38 mg, 53%). LCMS (m/z): [M/2+H]⁺ 357

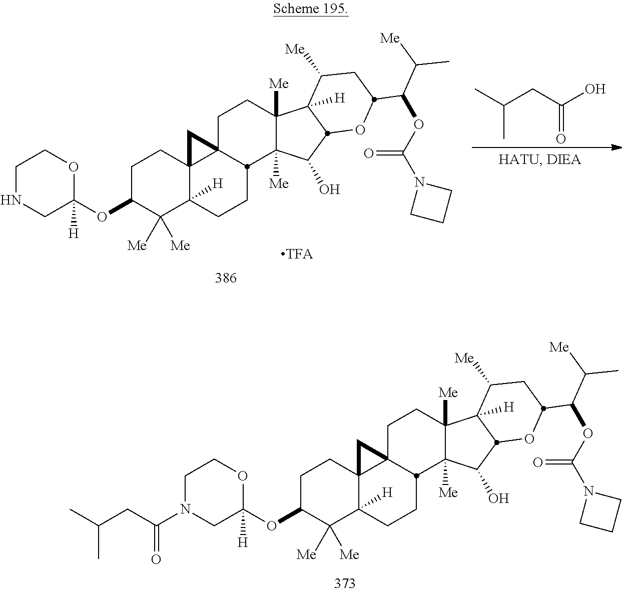

Procedure:

A mixture of amine 386 (10 mg, crude, 14 μmol), HATU (8 mg, 20 μmol), DIEA (6 mg, 42 μmol) and 3-methyl-butyric acid (3 mg, 20 μmol) in CH₂Cl₂ (0.5 mL) was stirred at room temperature overnight. The reaction mixture was diluted with CH₂Cl₂ (5 mL) and washed with saturated citri acid (5 mL). The phases were separated and the aqueous phase was extracted with CH₂Cl₂ (2 mL×2). The combined organic phase was dried over Na₂SO₄, filtered and concentrated in vacuo to give amide 373 (3.98 mg, 47%) which was purified by preparative HPLC. LCMS (m/z): [M/2+H]⁺ 364, [M+H]⁺ 727.5.

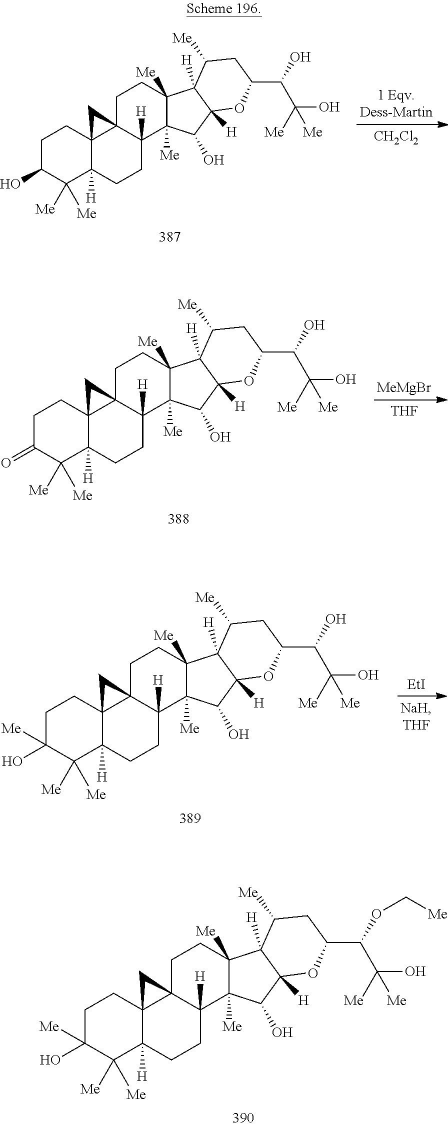

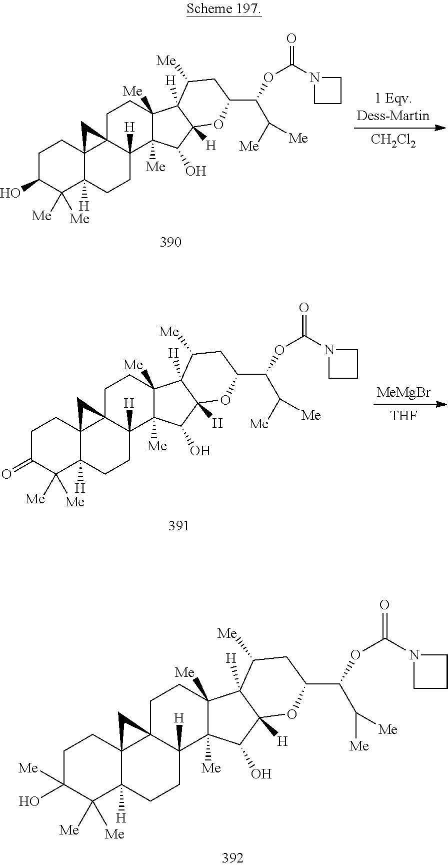

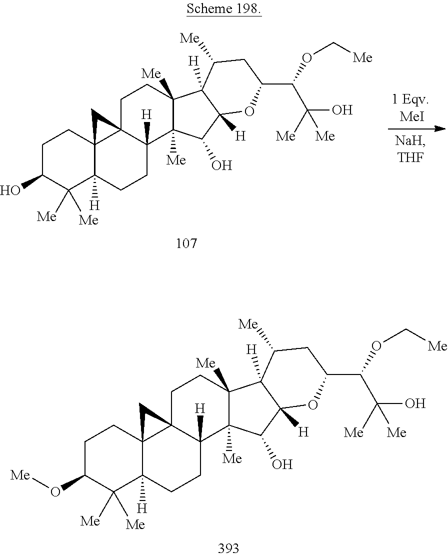

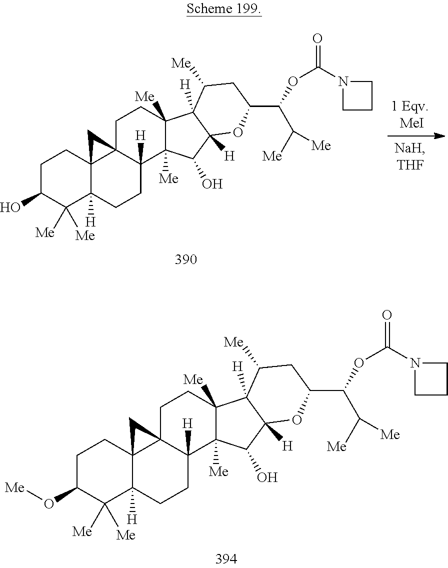

### Example 17

TABLE 44
Compounds

| | |
|---|---|
| 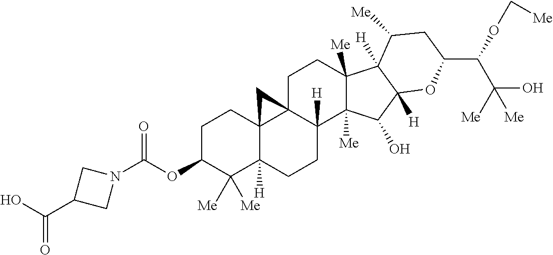 | 395 |
| 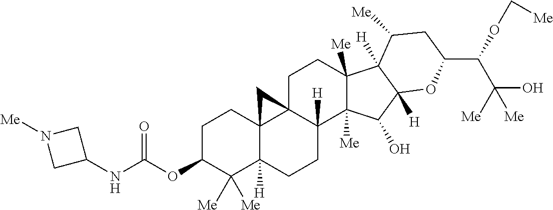 | 396 |

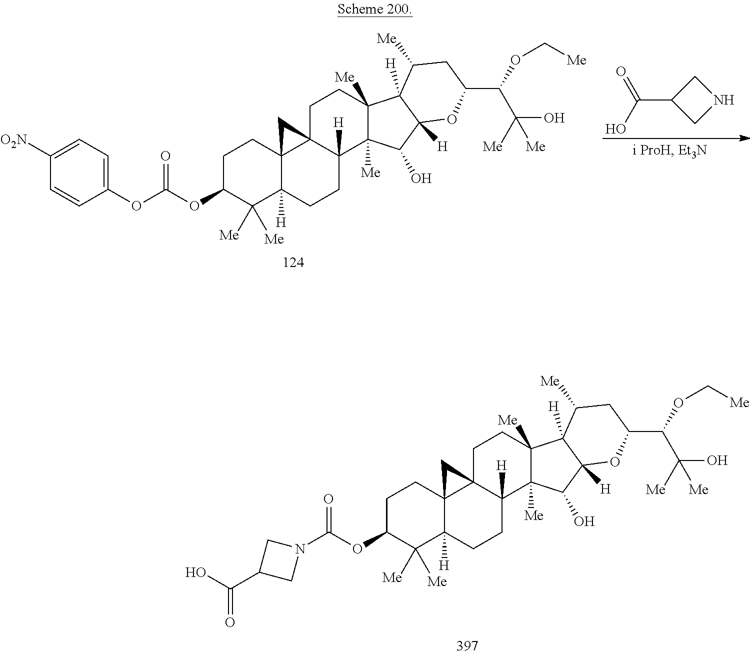

Compound 124 (8 mg) was dissolved in iPrOH (2 mL) and CH₂Cl₂ (1 mL) and 3-azetidinecarboxylic acid (7 mg) and Et₃N (12 μL) was added. The solution was stirred vigorously for 16 h. The solution was diluted in CH₂Cl₂ (15 mL) and washed with aq. conc. HCl (5 mL) and then twice with 10% NaHCO₃ (5 mL). The organic layer was removed in vacuo and the residue was purified by C18 chromatography (20-70% ACN/H₂O (0.1% HCO₂H)) to give compound 397 [m/z=668 (M⁺+Na)].

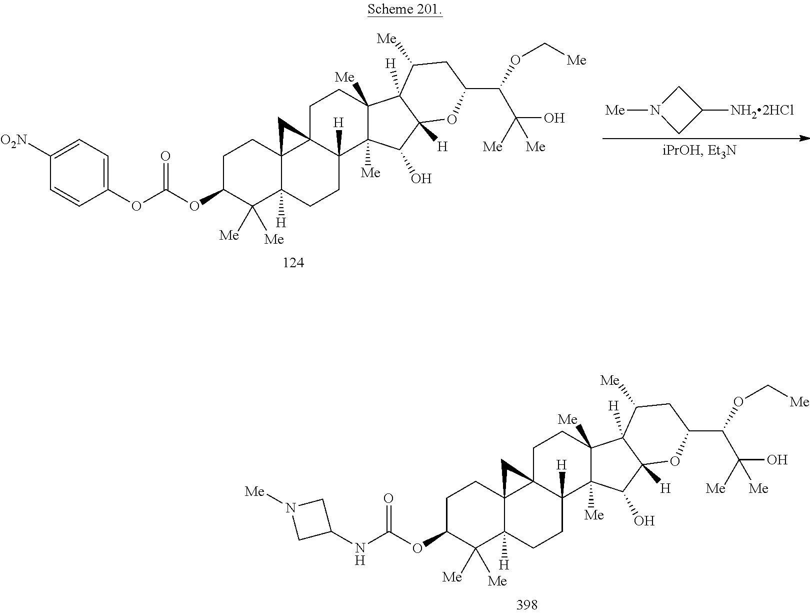

Compound 124 (8 mg) was dissolved in iPrOH (2 mL) and CH₂Cl₂ (1 mL) and 1-methyl-azetidin-3-ylamine dihydrochloride (10 mg) and Et₃N (18 μL) was added. The solution was stirred vigorously for 16 h. The solution was diluted in CH₂Cl₂ (15 mL) and washed with aq. conc. HCl (5 mL) and then twice with 10% NaHCO₃ (5 mL). The organic layer was removed in vacuo and the residue was purified by C18 chromatography (20-70% ACN/H₂O (0.1% HCO₂H)) to give compound 398 [m/z=632 (M⁺+Na)].

TABLE 45
Compounds
E-71

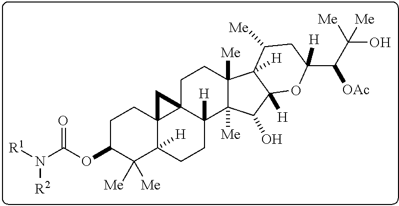

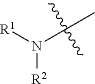

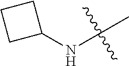 [SMILES: *NC1CCC1]

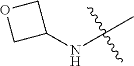 [SMILES: *NC1COC1]

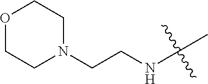 [SMILES: *NCCN1CCOCC1]

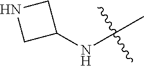 [SMILES: *NC1CNC1]

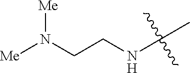 [SMILES: *NCCN(C)C]

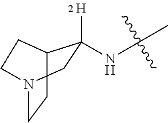 [SMILES: *NC1CN2CCC1CC2]

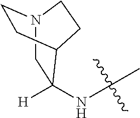 [SMILES: *NC1CN2CCC1CC2]

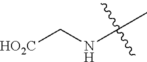 [SMILES: *NCC(=O)O]

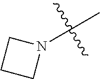 [SMILES: *N1CCC1]

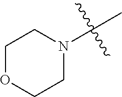 [SMILES: *N1CCOCC1]

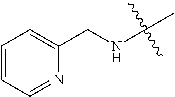 [SMILES: *NCc1ccccn1]

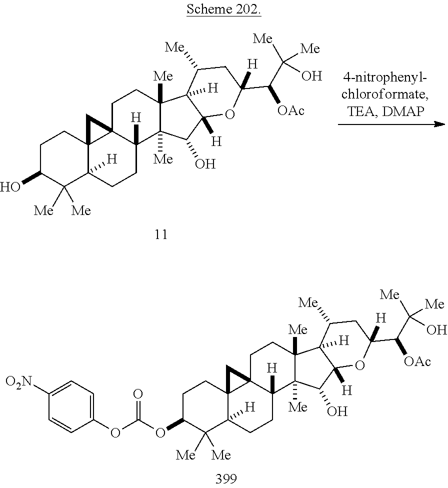

Procedure:

4-Nitrophenyl chloroformate (796 mg, 3.94 mmol) was added to Hunig's base (2.01 mL, 11.3 mmol), DMAP (480 mg, 3.94 mmol) and triol 11 (2.00 g, 3.75 mmol) in dry DCM (20 mL) under N₂ and allowed to stir for 1 h. TLC (EA) shows conversion to a less polar spot, >50%. The solution was loaded directly on a 50 g silica biotage column and eluted with 20-100% EA/Hex to give 305 mg pure 399. Repurification gave an additional 200 mg. Recovered triol 11 was resubjected to the reaction conditions to yield an addition 470 mg after purification.

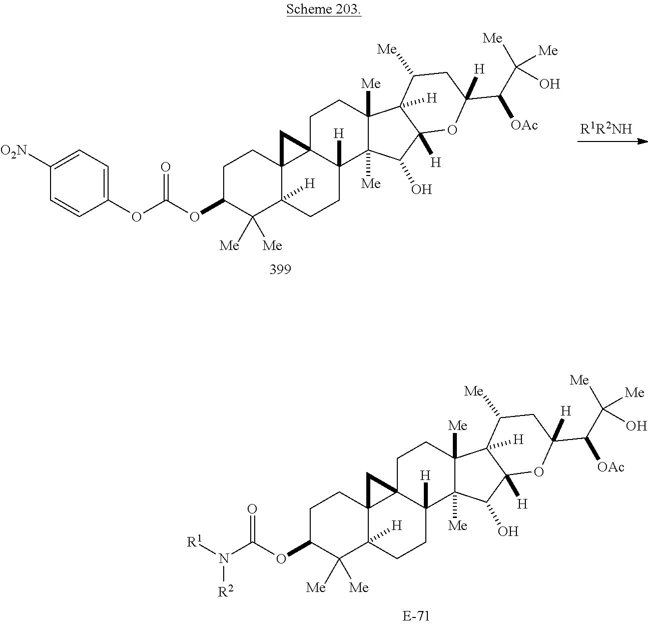

Sample Procedure A:

A solution of the mixed carbamate in DCM and/or ethanol was treated with 5 equiv. of amine (R¹R²NH) at between room temperature and 100° C. for between 1 h and 18 h. The solvent was removed and the product was purified by normal phase biotage chromatography, or by reverse phase HPLC.

Sample Procedure B:

1-Boc-3-aminoazetidine (61 mg) was added to the mixed carbonate (50 mg) at room temperature in EtOH and stirred overnight at 50° C. and TLC indicated complete conversion to a more polar spot. The intermediate was purified by chromatography (20-100% EA/Hex 10 g biotage column). The purified Boc carbamate was dissolved in 1 mL DCM then 1 mL TFA was added, and the solution was allowed to stir for 2 h. LC/MS shows no remaining starting material. The solution was partitioned between NaHCO₃ sat aq. and DCM. DCM solution was dried over Na₂SO₄, and concentrated. Gave 20 mg.

Sample Procedure C:

Glycine t-butyl ester (47 mg mg) was added to the mixed carbonate (50 mg) at room temperature in EtOH (1 mL) and stirred overnight at 50° C. and TLC indicated complete conversion to a more polar spot. The intermediate was purified by chromatography (20-100% EA/Hex 10 g biotage column). The purified tert-butyl ester was dissolved in 1 mL DCM then 1 mL TFA was added, and the solution was allowed to stir for 2 h. LC/MS shows no remaining starting material. The solution was partitioned between 1 N HCl and DCM. DCM solution was dried over Na₂SO₄, and concentrated. Gave 30 mg.

TABLE 46
Compounds
E-72

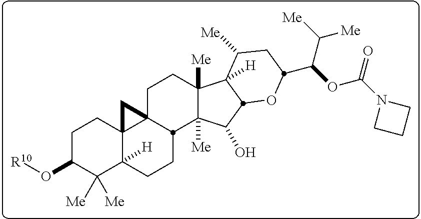

R¹⁰ =

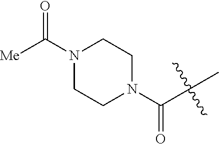 [SMILES: *C(=O)N1CCN(C(C)=O)CC1]

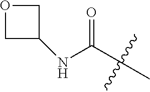 [SMILES: *C(=O)NC1COC1]

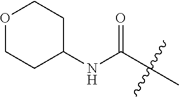 [SMILES: *C(=O)NC1CCOCC1]

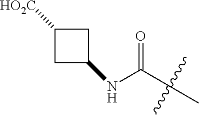 [SMILES: *C(=O)N[C@H]1C[C@H](C(=O)O)C1]

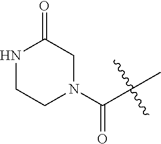 [SMILES: *C(=O)N1CCNC(=O)C1]

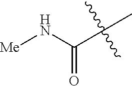 [SMILES: *C(=O)NC]

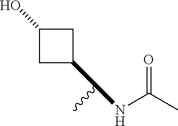

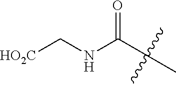 [SMILES: *C(=O)NCC(=O)O]

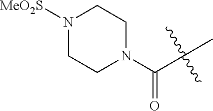 [SMILES: *C(=O)N1CCN(S(C)(=O)=O)CC1]

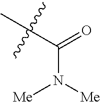 [SMILES: *C(=O)N(C)C]

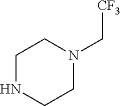 [SMILES: FC(F)(F)CN1CCNCC1]

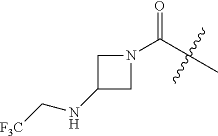 [SMILES: *C(=O)N1CC(NCC(F)(F)F)C1]

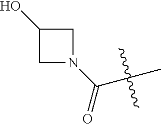 [SMILES: *C(=O)N1CC(O)C1]

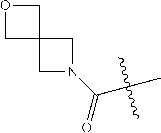 [SMILES: *C(=O)N1CC2(COC2)C1]

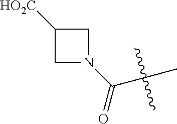 [SMILES: *C(=O)N1CC(C(=O)O)C1]

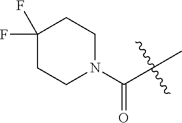 [SMILES: *C(=O)N1CCC(F)(F)CC1]

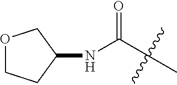 [SMILES: *C(=O)N[C@H]1CCOC1]

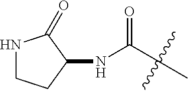 [SMILES: *C(=O)N[C@H]1CCNC1=O]

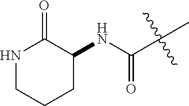 [SMILES: *C(=O)N[C@H]1CCCNC1=O]

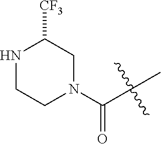 [SMILES: *C(=O)N1CCN[C@H](C(F)(F)F)C1]

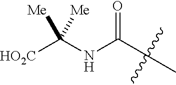

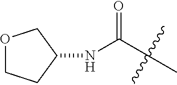 [SMILES: *C(=O)N[C@@H]1CCOC1]

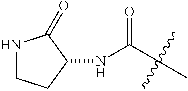 [SMILES: *C(=O)N[C@@H]1CCNC1=O]

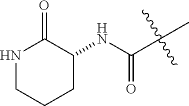 [SMILES: *C(=O)N[C@@H]1CCCNC1=O]

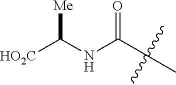 [SMILES: *C(=O)N[C@H](C)C(=O)O]

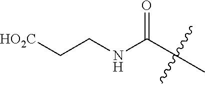 [SMILES: *C(=O)NCCC(=O)O]

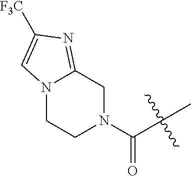 [SMILES: *C(=O)N1CCn2cc(C(F)(F)F)nc2C1]

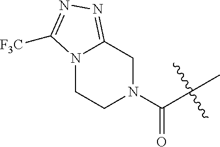 [SMILES: *C(=O)N1CCn2c(nnc2C(F)(F)F)C1]

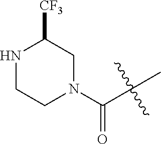 [SMILES: *C(=O)N1CCN[C@@H](C(F)(F)F)C1]

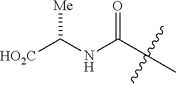 [SMILES: *C(=O)N[C@@H](C)C(=O)O]

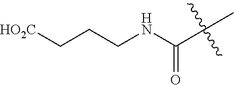 [SMILES: *C(=O)NCCCC(=O)O]

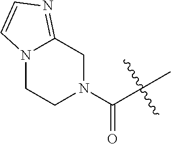 [SMILES: *C(=O)N1CCn2ccnc2C1]

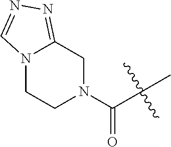 [SMILES: *C(=O)N1CCn2cnnc2C1]

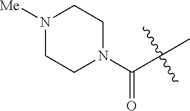 [SMILES: *C(=O)N1CCN(C)CC1]

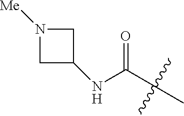 [SMILES: *C(=O)NC1CN(C)C1]

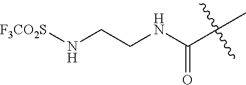

TABLE 47
Compounds
E-73

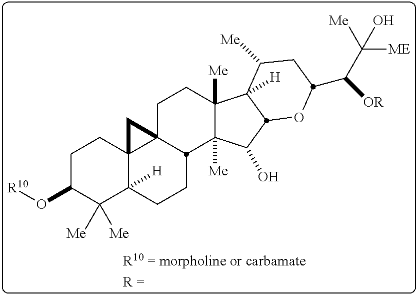

CH₂CF₃
iPr

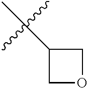 [SMILES: *C1COC1]

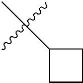 [SMILES: *C1CCC1]

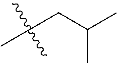 [SMILES: *CC(C)C]

### Exemplary Compounds:

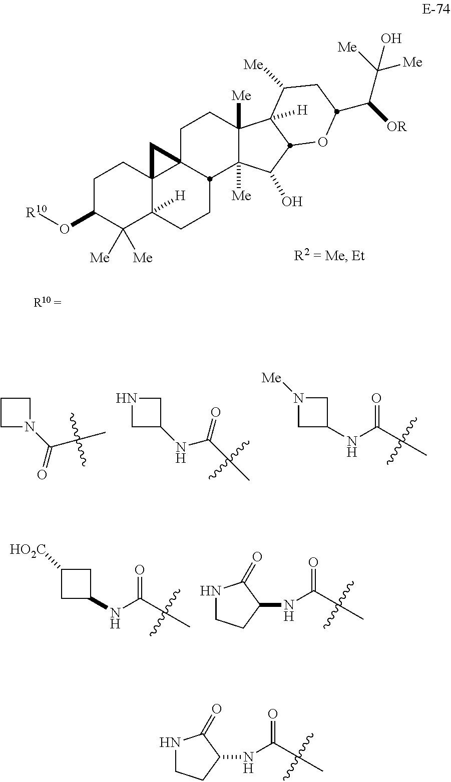

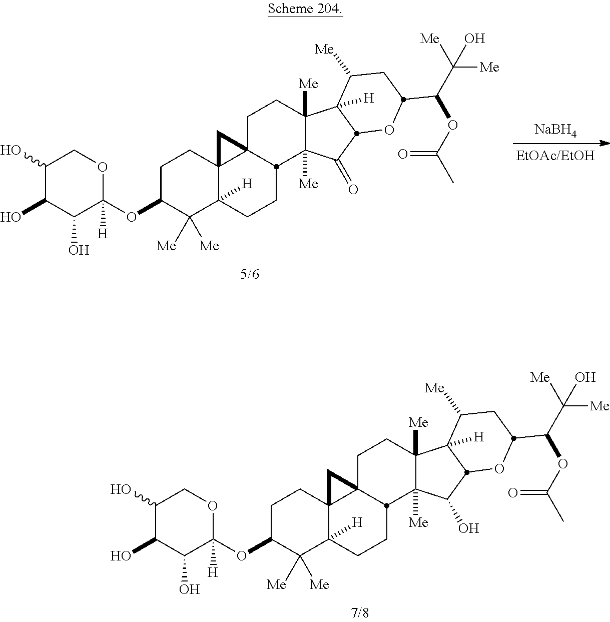

Procedure:

Sodium borohydride (856 mg, 0.023 mmol) was added to ethanol (20 mL) in a 500 mL RBF and allowed to stir for 10 min. EtOAc (100 mL) was added followed by compound 5 and 6 (10 g, 0.015 mmol) at room temperature. After 1 h LC/MS shows good conversion and some acetate cleavage. HCl was added carefully over several minutes with cooling in an ice bath (evolution of hydrogen!). The solution was stirred for 10 min and partitioned between 400 mL each CH₂Cl₂ and water. The layers were separated and the aqueous layer was extracted with CH₂Cl₂ (200 mL×2) and the combined organic layers were dried over Na₂SO₄, filtered and concentrated under reduced pressure to give the crude product (10 g) which was used for the next step without further purification

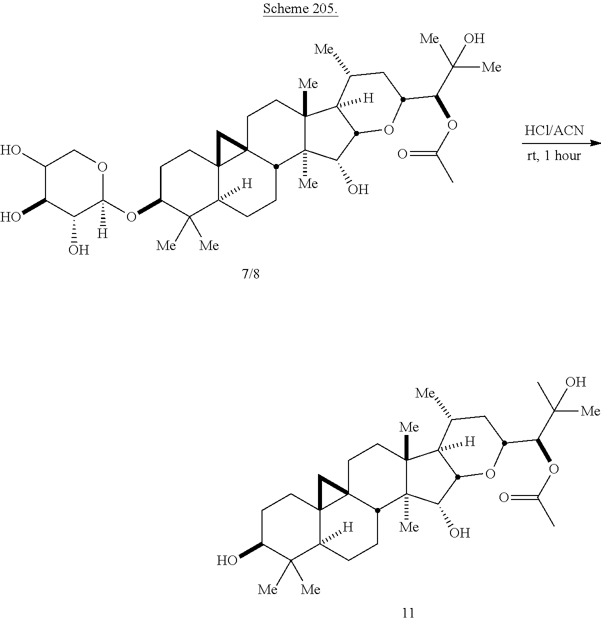

Procedure:

HCl (conc., 20 mL) was added to crude 7 and 8 (10 g, 0.015 mmol) in CH₃CN (80 mL) and allowed to stir for 1 h at room temperature. The reaction mixture was partitioned between 400 mL each CH₂CL₂ and water. The organic layer was washed with NaHCO₃, dried over Na₂SO₄, filtered and concentrated to give the residue, which was purified by biotage chromatography DCM:MeOH=100:1) to give pure acetate 11 (2.5 g, 31%).

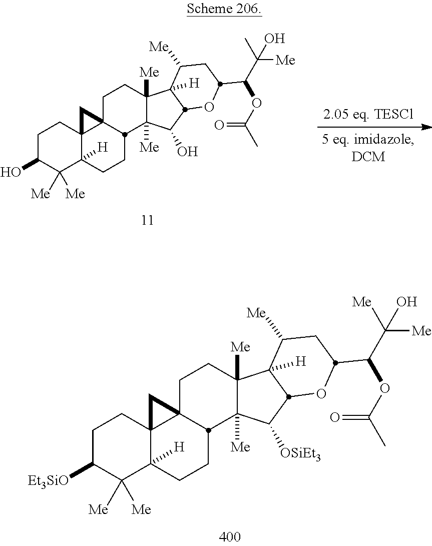

Procedure:

TESCl (1.45 g, 9.62 mmol) was added to triol 11 (2.5 g, 4.69 mmol) followed by imidazole (1.59 g, 23.45 mmol) in DCM (10 mL). TLC shows good balance of conversion and tri-protection after 1 hour. Water (50 mL) was added and the mixture was extracted with DCM (20 mL×3). The combined organic layers were washed with brine, dried over Na₂SO₄, filtered and concentrated to give 400, which was purified by chromatography (PE:EA=100:1) (2.3 g, 65%).

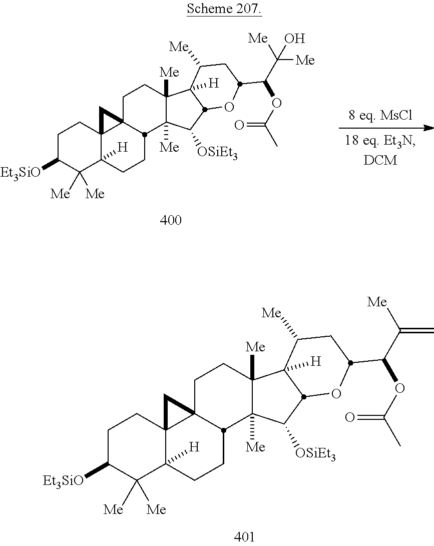

Procedure:

To a solution of alcohol 400 (2.3 g, 3.03 mmol) and Et₃N (5.51 g, 54.54 mmol) in dry DCM (10 mL) was added MsCl (2.71 g, 24.21 mmol) dropwise slowly in ice water bath. Then the mixture was stirred at room temperature for 1 hour. TLC showed the reaction was completed. Water (50 mL) was added and the mixture was extracted with DCM (20 mL×3). The combined organic layers was washed with brine, dried over Na₂SO₄, filtered and concentrated to give alkene 401 which was purified by column chromatography (PE:EA=200:1) (1.37 g, 61%).

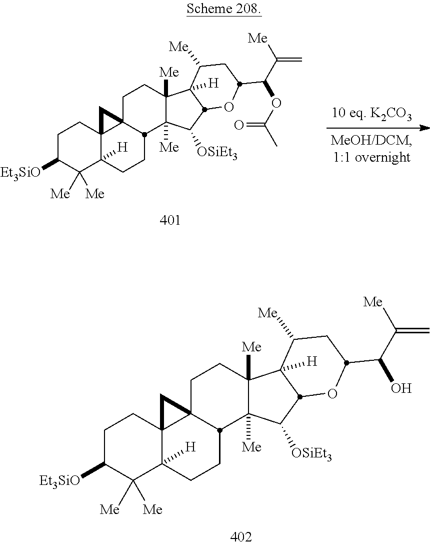

Procedure:

To a solution of alkene 401 (1.37 g, 1.85 mmol) in DCM (15 mL) and MeOH (15 mL) was added K₂CO₃ (2.49 g, 18.5 mmol). Then the mixture was stirred at room temperature for 12 hours. TLC showed the reaction was completed. The mixture was concentrated and dissolved in DCM (150 mL). The organic layer was washed with water (20 mL×3), brine, dried over Na₂SO₄, filtered and concentrated to give alcohol 402, which was used for the next step directly (1.28 g, 99%).

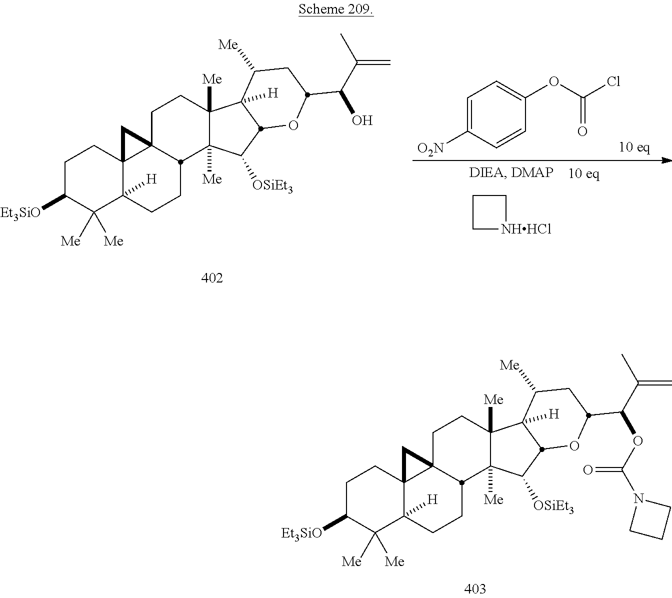

Procedure:

4-Nitrophenyl chloroformate (860 mg, 4.28 mmol) was added to DIEA (277 mg, 2.14 mmol), DMAP (523 mg, 4.28 mmol) and alcohol 402 (300 mg, 0.43 mmol) in dry CH₂Cl₂ (2 mL) under N₂ and allowed to stir for 12 hours. Azetidine hydrochloride salt (200 mg, 2.15 mmol) was added. Then the mixture was stirred at room temperature for another 2 hours. TLC showed the reaction was completed. Water (30 mL) was added and the mixture was extracted with DCM (15 mL×3). The organic layers were combined, washed with brine, dried over Na₂SO₄, filtered and concentrated to give carbamate 403 which was purified by column chromatography (180 mg, 54%).

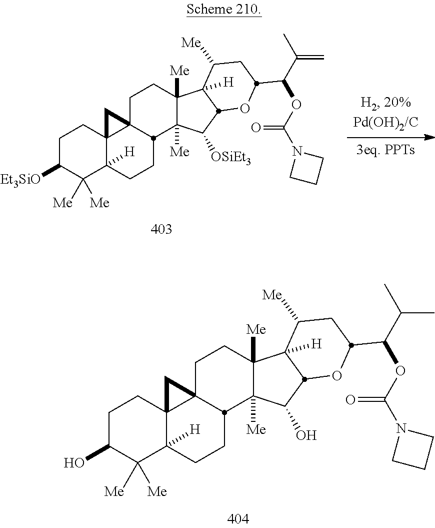

Procedure:

A solution of alkene 403 (180 mg, 0.23 mmol) in MeOH (10 mL) and EA (2 mL) was treated with 20% Pd(OH)₂ on carbon (wet) (36 mg). The reaction mixture was stirred under H₂ (1 atm) at room temperature for 30 min. TLC showed the reaction was finished. Then the mixture was filtered and solvent was removed in vacuo to give the residue. To the residue in DCM (2 mL) and MeOH (2 mL) was added PPT_{S} (173 mg, 0.69 mmol). Then the mixture was stirred at room temperature for 30 minutes. TLC showed the reaction was completed. Solvent was removed in vacuo and the obtained residue was purified by column chromatography to give carbamate 404 (120 mg, 94%).

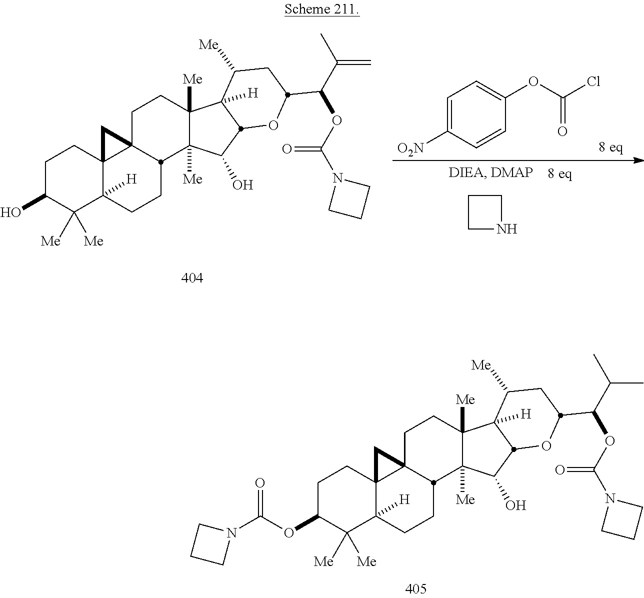

Procedure:

4-Nitrophenyl chloroformate (59 mg, 0.29 mmol) was added to DIEA (23 mg, 0.18 mmol), DMAP (35 mg, 0.29 mmol) and carbamate 404 (20 mg, 0.036 mmol) in dry CH₂Cl₂ (1 mL) under N₂ and allowed to stir for 12 hours. TLC showed that the starting material was disappeared. Azetidine hydrochloride salt (6 mg, 0.11 mmol) was added. Then the mixture was stirred at room temperature for another 2 hours. TLC showed the reaction was completed. Water (15 mL) was added and the mixture was extracted with DCM (15 mL×3). The organic layers were combined, washed with brine, dried over Na₂SO₄, filtered and concentrated to give bis-carbamate 405 which was purified by preparative HPLC (7.84 mg, 34%). LCMS (m/z): [M+H]⁺ 641.3

Using the same method, the following products were obtained:

TABLE 48
Compounds

| | |
|---|---|
| 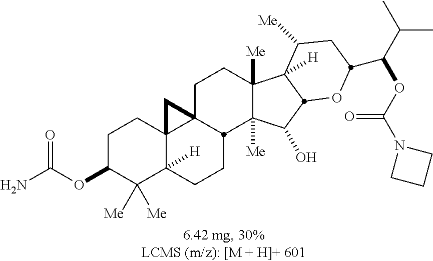 | 406 |
| 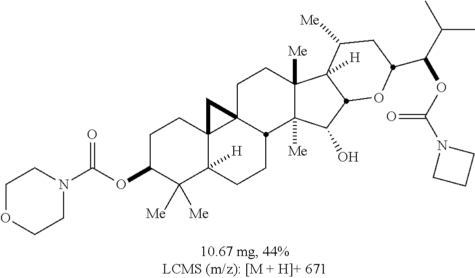 | 407 |
| 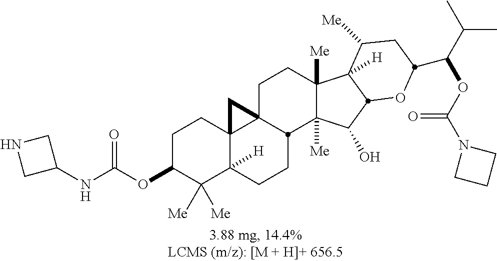 | 408 |
| 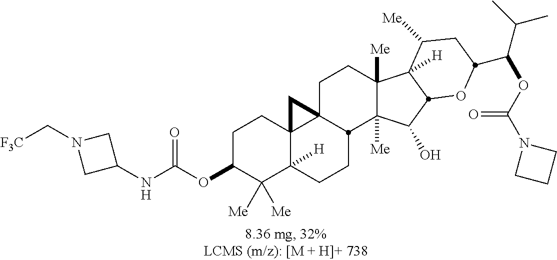 | 409 |

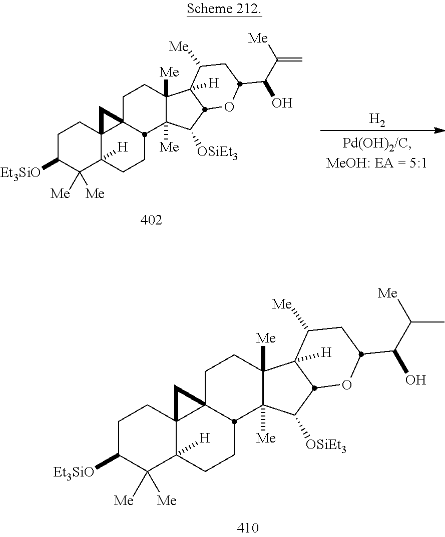

Procedure:

A solution of alkene 402 (500 mg, 0.71 mmol) in MeOH (20 mL) and EA (4 mL) was treated with 20% Pd(OH)₂ on carbon (wet) (50 mg). The reaction mixture was stirred under H₂ (1 atm) at room temperature for 30 min. TLC showed the reaction was finished. Then the mixture was filtered and solvent was removed in vacuo to give alcohol 410, which was used in the next step without purification (500 mg, crude).

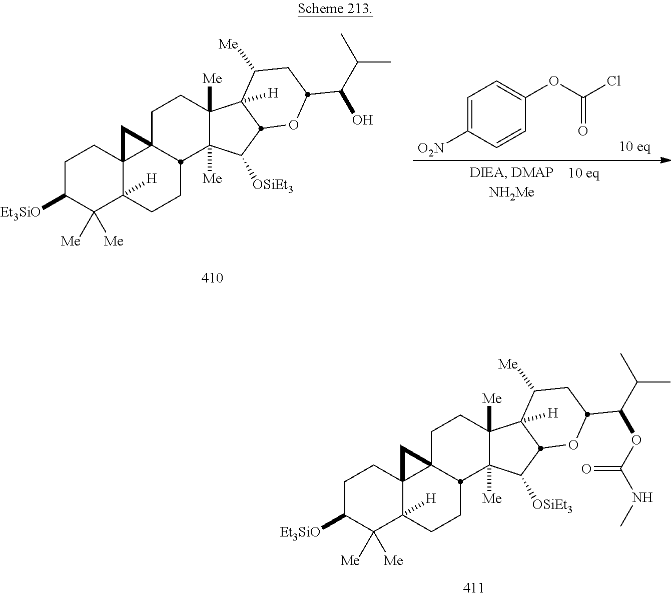

Procedure:

4-Nitrophenyl chloroformate (573 mg, 2.85 mmol) was added to Et₃N (571 mg, 5.7 mmol), DMAP (348 mg, 2.85 mmol) and alcohol 410 (200 mg, 0.285 mmol) in dry CH₂Cl₂ (2 mL) under N₂ and allowed to stir for 12 hours. MeNH₂.HCl (87 mg, 1.43 mmol) was added. Then the mixture was stirred at room temperature for another 2 hours. TLC showed the reaction was completed. Water (30 mL) was added. The aqueous layer was extracted with DCM (15 mL×3). The organic layers were combined, washed with brine, dried over Na₂SO₄, filtered and concentrated to give carbamate 411, which was purified by chromatography (150 mg, 74%).

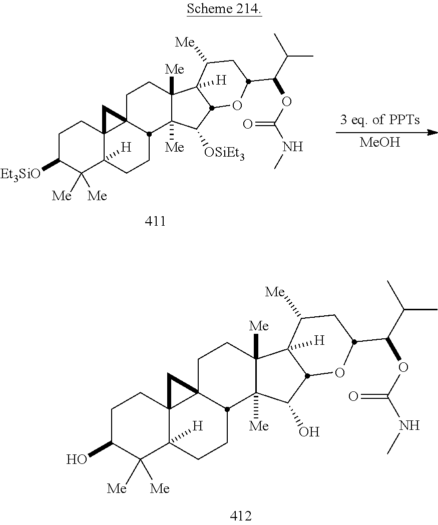

Procedure:

To a solution of carbamate 411 (150 mg, 0.198 mmol) in DCM (2 mL) and MeOH (2 mL) was added PPTS (149 mg, 0.592 mmol,). Then the mixture was stirred at room temperature for 30 minutes. TLC showed the reaction was completed. Solvent was removed in vacuo to give carbamate 412, which was purified by chromatography (100 mg, 95%).

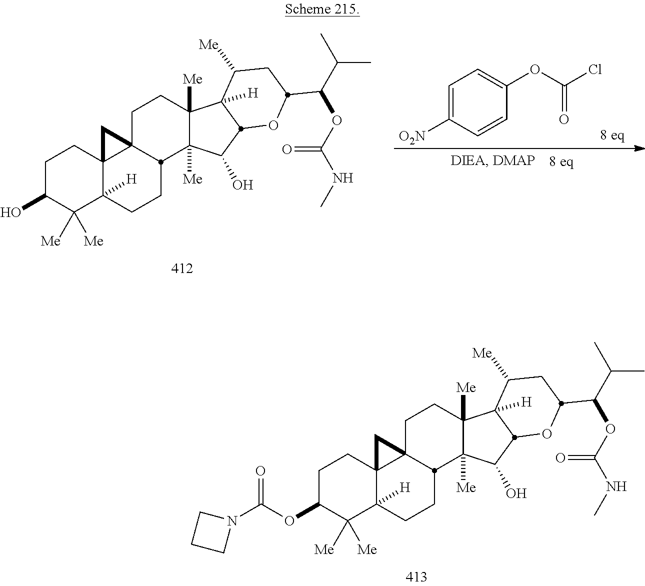

Procedure:

4-Nitrophenyl chloroformate (60 mg, 0.3 mmol) was added to DIEA (24 mg, 0.189 mmol), DMAP (37 mg, 0.3 mmol) and alcohol 412 (20 mg, 0.0377 mmol) in dry CH₂Cl₂ (1 mL) under N₂ and allowed to stir for 12 hours. TLC showed that the starting material was disappeared. Azetidine hydrochloride salt (11 mg, 0.113 mmol) was added. Then the mixture was stirred at room temperature for another 2 hours. TLC showed the reaction was completed. Water (30 mL) was added. The aqueous layer was extracted with DCM (15 mL×3). The organic layers were combined, washed with brine, dried over Na₂SO₄, filtered and concentrated to give the residue, which was purified by HPLC to give alcohol 413 (14.42 mg, 63%). LCMS (m/z): [M+H]⁺ 615

Using the same method, the following products were obtained:

TABLE 49
Compounds

| | |
|---|---|
| 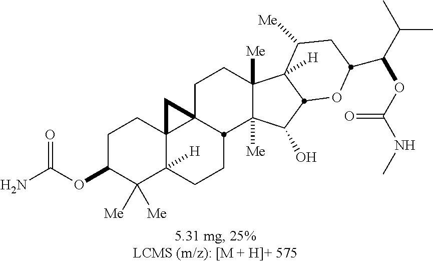 | 414 |
| 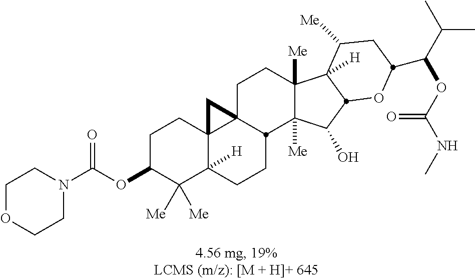 | 415 |
| 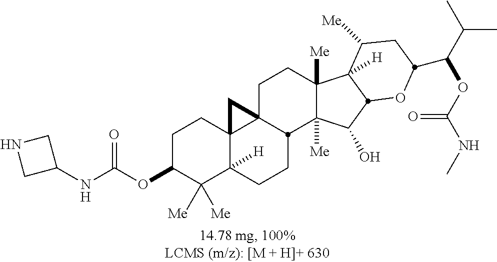 | 416 |
| 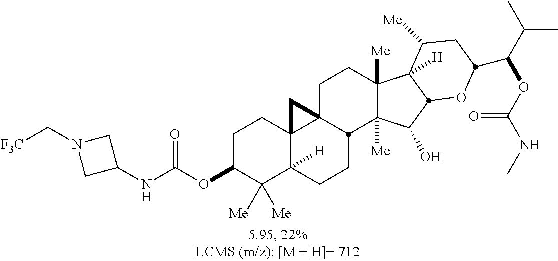 | 417 |

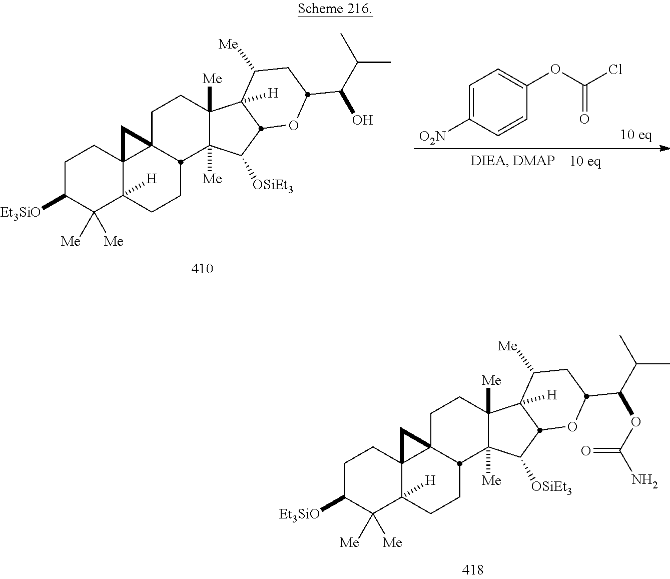

Procedure:

4-Nitrophenyl chloroformate (859 mg, 4.3 mmol) was added to DIEA (278 mg, 2.15 mmol), DMAP (525 mg, 4.3 mmol) and alcohol 410 (300 mg, 0.43 mmol) in dry CH₂Cl₂ (1 mL) under N₂ and allowed to stir for 12 hours. Then the mixture was stirred at room temperature under NH₃ for another 2 hours. TLC showed the reaction was completed. Water (30 mL) was added. The aqueous layer was extracted with DCM (15 mL×3). The organic layers were combined, washed with brine, dried over Na₂SO₄, filtered and concentrated to give the residue, which was purified by chromatography to give carbamate 418 (220 mg, 74%).

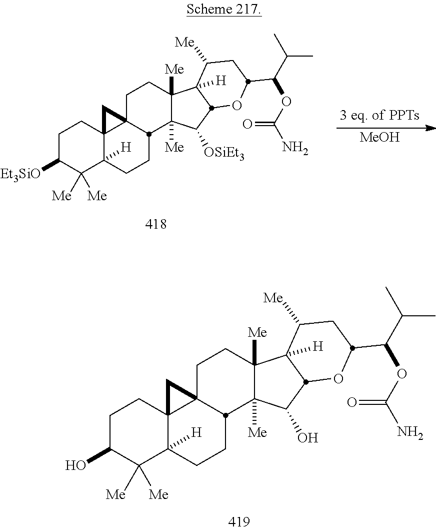

Procedure:

To a solution of silyl ether 418 (220 mg, 0.3 mmol) in DCM (2 mL) and MeOH (2 mL) was added PPTS (222 mg, 0.9 mmol,). Then the mixture was stirred at room temperature for 30 minutes. TLC showed the reaction was completed. Solvent was removed in vacuo to give diol 419 which was purified by chromatography (120 mg, 78%).

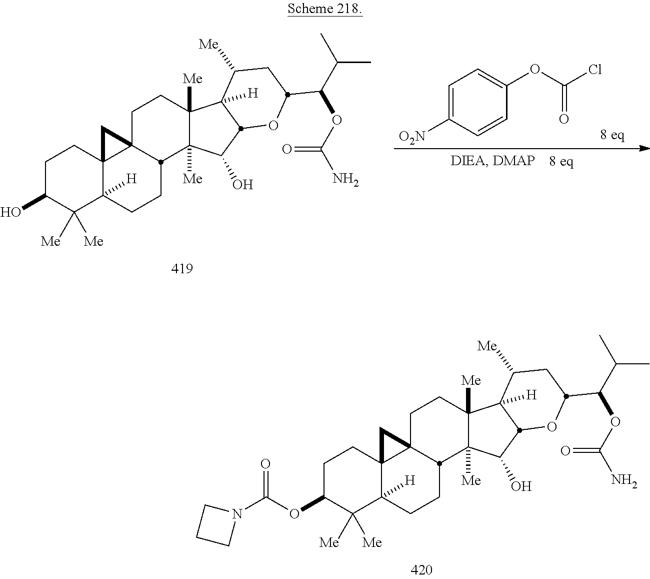

Procedure:

4-Nitrophenyl chloroformate (75 mg, 0.37 mmol) was added to DIEA (30 mg, 0.23 mmol), DMAP (45 mg, 0.37 mmol) and alcohol 419 (24 mg, 0.046 mmol) in dry CH₂Cl₂ (1 mL) under N₂ and allowed to stir for 12 hours. Azetidine hydrochloride salt (21 mg, 0.37 mmol) was added. Then the mixture was stirred at room temperature for another 2 hours. TLC showed the reaction was completed. Water (30 mL) was added. The aqueous layer was extracted with DCM (15 mL×3). The organic layers were combined, washed with brine, dried over Na₂SO₄, filtered and concentrated to give the residue, which was purified by HPLC to give alcohol 420 (4.34 mg, 12%). LCMS (m/z): [M+H]⁺601

Using the same method, the following products were obtained:

TABLE 50
Compounds

| | |
|---|---|
| 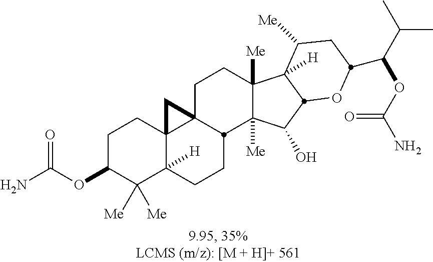 | 421 |
| 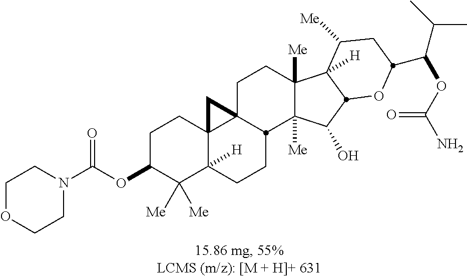 | 422 |
| 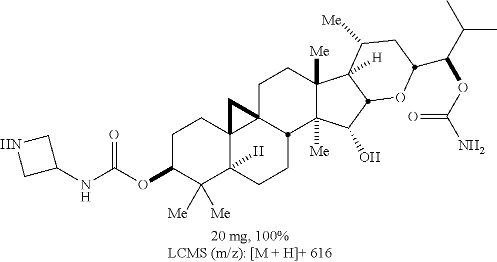 | 423 |
| 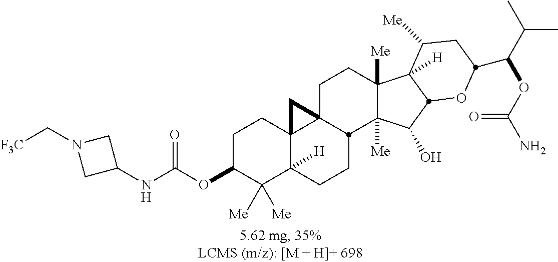 | 424 |

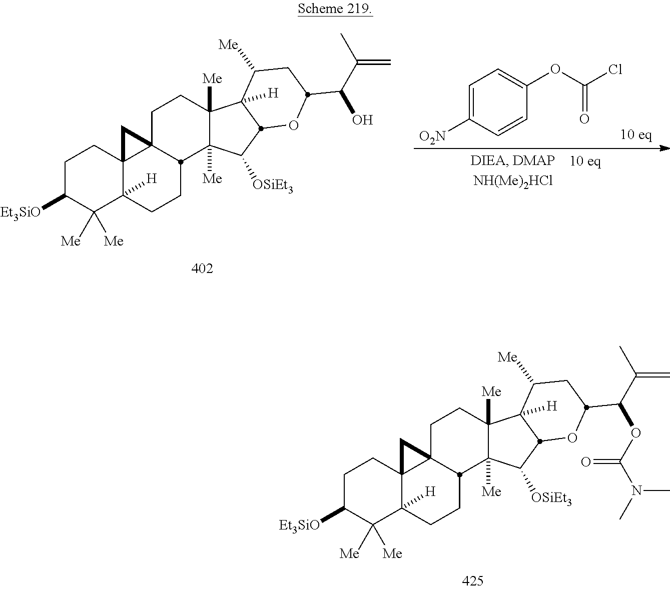

Procedure:

4-Nitrophenyl chloroformate (860 mg, 4.28 mmol) was added to DIEA (277 mg, 2.14 mmol), DMAP (523 mg, 4.28 mmol) and alkene 402 (300 mg, 0.43 mmol) in dry CH₂Cl₂ (2 mL) under N₂ and allowed to stir for 12 hours. (Me)₂NH.HCl (87 mg, 1.07 mmol) was added. Then the mixture was stirred at room temperature for another 2 hours. TLC showed the reaction was completed. Water (30 mL) was added and the mixture was extracted with DCM (15 mL×3). The organic layers were combined, washed with brine, dried over Na₂SO₄, filtered and concentrated to give carbamate 425 which was purified by column chromatography (170 mg, 51.5%).

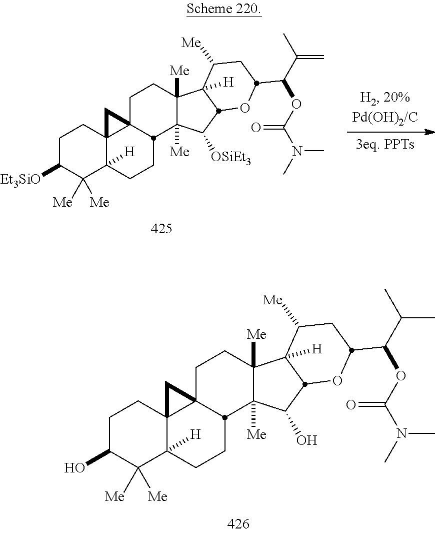

Procedure:

A solution of alkene 425 (170 mg, 0.22 mmol) in MeOH (10 mL) and EA (2 mL) was treated with 20% Pd(OH)₂ on carbon (wet) (34 mg). The reaction mixture was stirred under H₂ (1 atm) at room temperature for 30 min. TLC showed the reaction was finished. Then the mixture was filtered and solvent was removed in vacuo to give the residue. To the residue in DCM (2 mL) and MeOH (2 mL) was added PPTS (166 mg, 0.66 mmol). Then the mixture was stirred at room temperature for 30 minutes. TLC showed the reaction was completed. Solvent was removed in vacuo and the obtained residue was purified by column chromatography to give carbamate 426 (not pure enough, 140 mg, 100%).

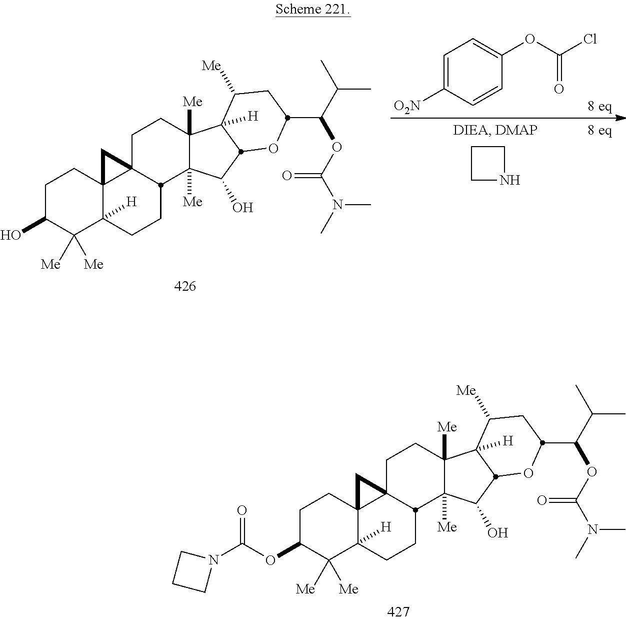

Procedure:

4-Nitrophenyl chloroformate (59 mg, 0.29 mmol) was added to DIEA (23 mg, 0.18 mmol), DMAP (35 mg, 0.29 mmol) and alcohol 426 (20 mg, 0.037 mmol) in dry CH₂Cl₂ (1 mL) under N₂ and allowed to stir for 12 hours. TLC showed that the starting material was disappeared. Azetidine hydrochloride salt (6 mg, 0.11 mmol) was added. Then the mixture was stirred at room temperature for another 2 hours. TLC showed the reaction was completed. Water (30 mL) was added and the mixture was extracted with DCM (15 mL×3). The organic layers were combined, washed with brine, dried over Na₂SO₄, filtered and concentrated to give carbamate 427 which was purified by preparative HPLC (10.67 mg, 46.4%). LCMS (m/z): [M+H]⁺ 629

Using the same method, the following products were obtained:

TABLE 51
Compounds

| | |
|---|---|
| 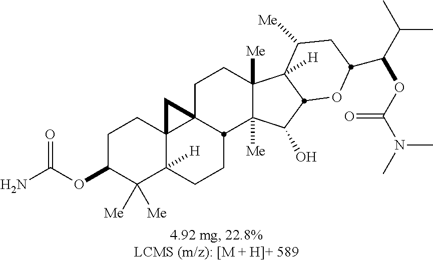 | 428 |
| 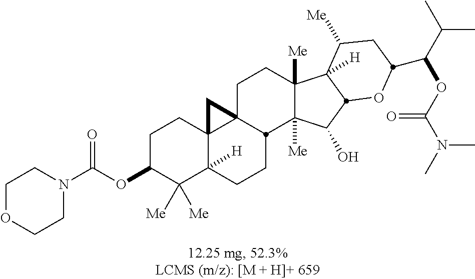 | 429 |
| 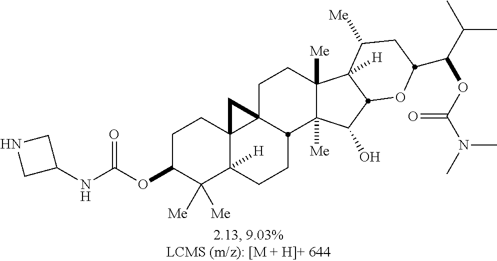 | 430 |
| 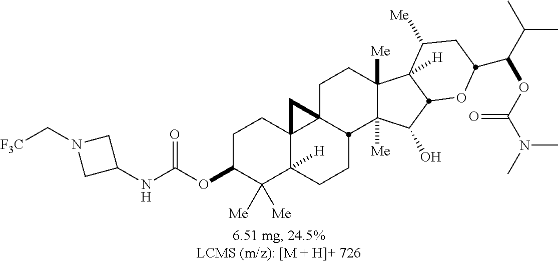 | 431 |

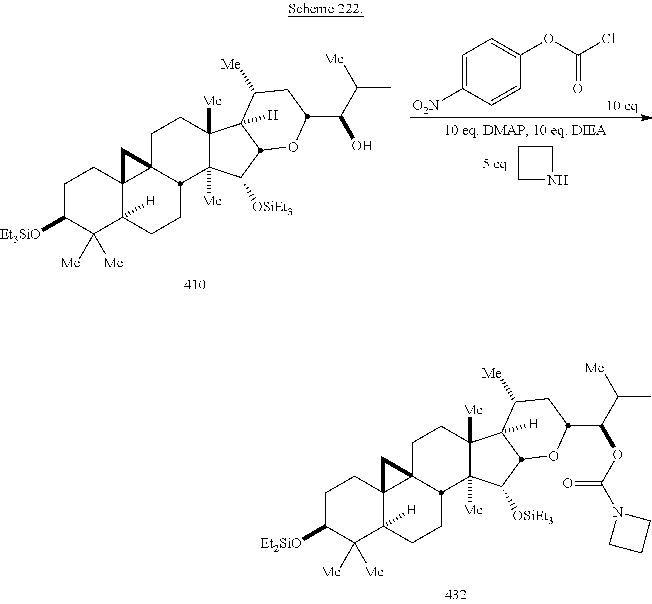

Procedure:

4-Nitrophenyl chloroformate (2.29 g, 11.4 mmol) was added to DIEA (1.47 mg, 11.4 mmol), DMAP (1.39 g, 11.4 mmol) and 410 (800 mg, 1.14 mmol) in dry CH₂Cl₂ (3 mL) under N₂ and allowed to stir for 12 hours. Azetidine hydrochloride salt (530 mg, 5.7 mmol) was added. Then the mixture was stirred at room temperature for another 1 hour. TLC showed the reaction was completed. Water (30 mL) was added and the mixture was extracted with DCM (15 mL×3). The organic layers were combined, washed with brine, dried over Na₂SO₄, filtered and concentrated to give carbamate 432, which was purified by column chromatography (410 mg, 46%).

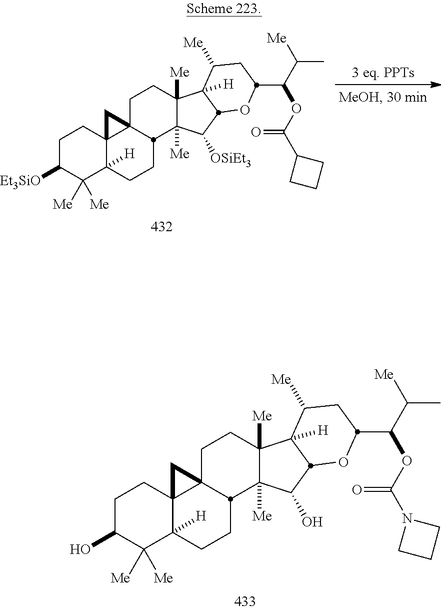

Procedure:

To a solution of carbamate 432 (410 mg, 0.52 mmol) in DCM (3 mL) and MeOH (3 mL) was added PPTS (393 mg, 1.57 mmol,). Then the mixture was stirred at room temperature for 30 minutes. TLC showed the reaction was completed. Solvent was removed in vacuo and the obtained residue was purified by column chromatography to give alcohol 433 (200 mg, 69%).

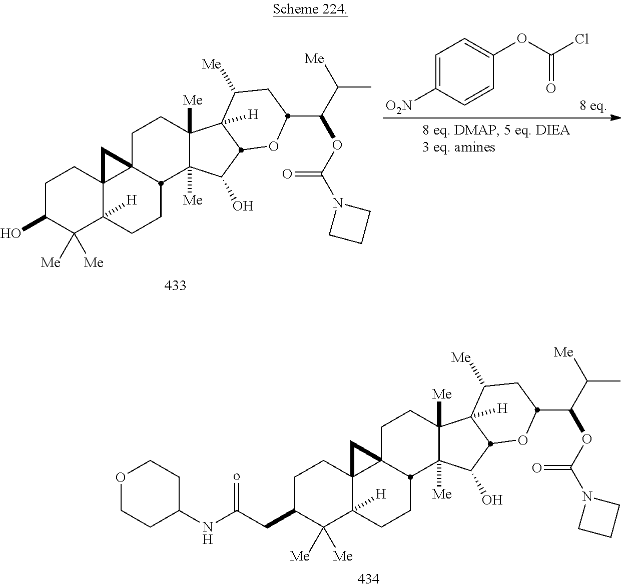

Procedure:

4-Nitrophenyl chloroformate (43 mg, 0.216 mmol) was added to DIEA (18 mg, 0.135 mmol), DMAP (26 mg, 0.216 mmol) and carbamate 433 (15 mg, 0.027 mmol) in dry CH₂Cl₂ (1 mL) under N₂ and allowed to stir for 12 hours. Tetrahydro-pyran-4-ylamine hydrochloride salt (11 mg, 0.081 mmol) was added. Then the mixture was stirred at room temperature for another 2 hours. TLC showed the reaction was completed. Water (15 mL) was added and the mixture was extracted with DCM (3×15 mL). The combined organic layers were dried over Na₂SO₄, filtered and concentrated to give SW-127 which was purified by preparative HPLC (9.07 mg, 49%). LCMS (m/z): [M+H]⁺ 685

Using the same method, the following products were obtained:

TABLE 52
Compounds

| | |
|---|---|
| 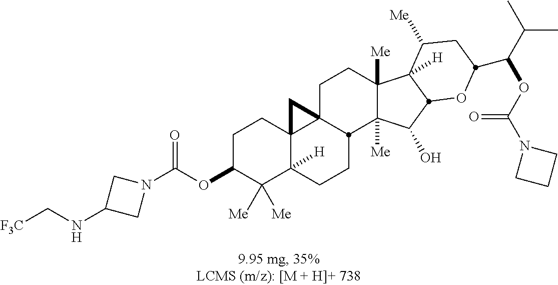 | 435 |
| 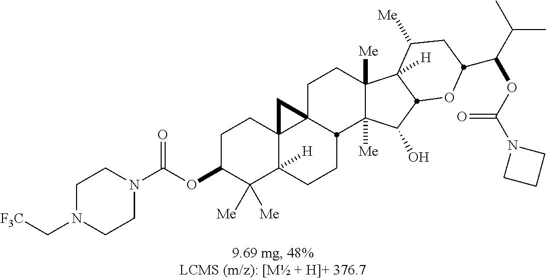 | 436 |
| 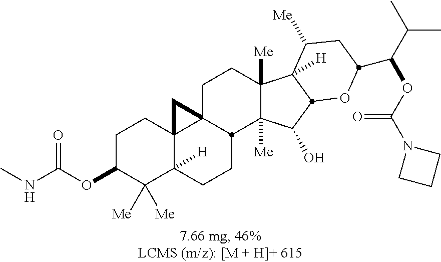 | 437 |
| 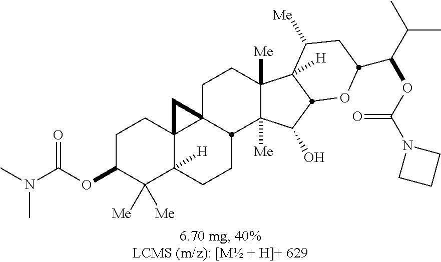 | 438 |
| 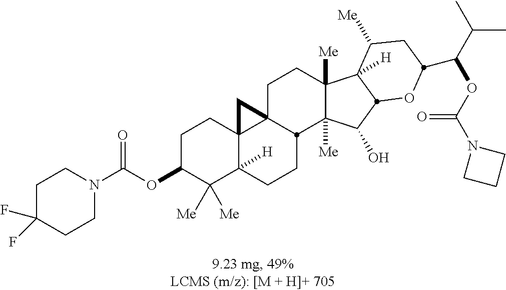 | 439 |
| 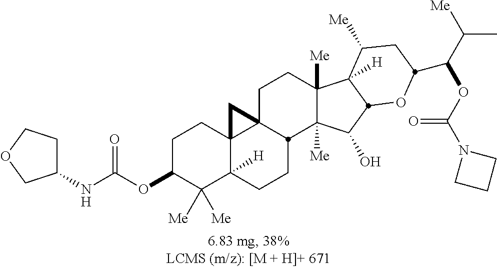 | 440 |
| 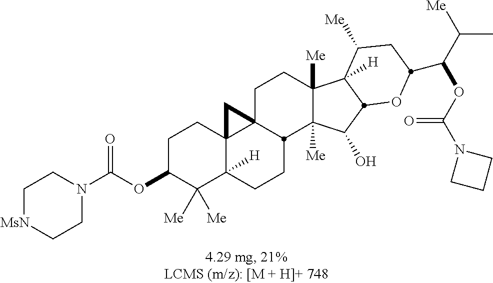 | 441 |
| 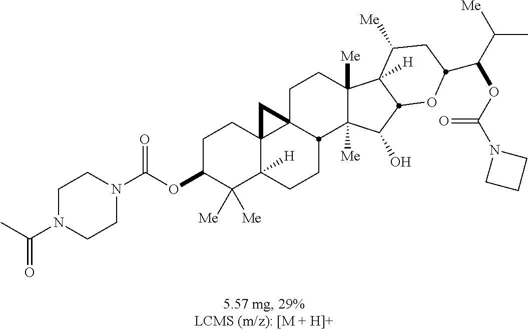 | 442 |
| 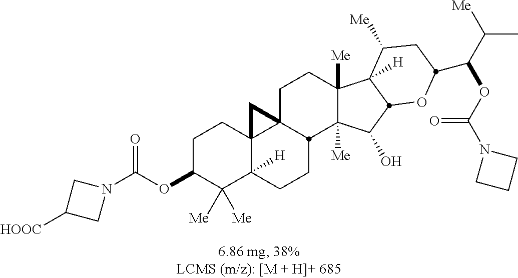 | 443 |
| 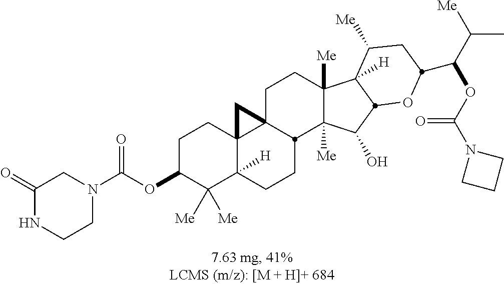 | 444 |
| 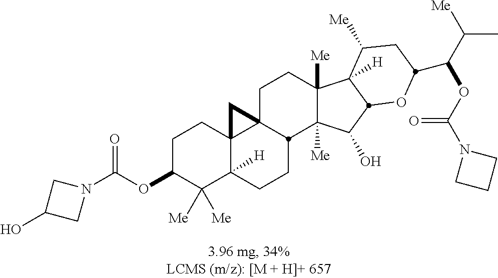 | 445 |

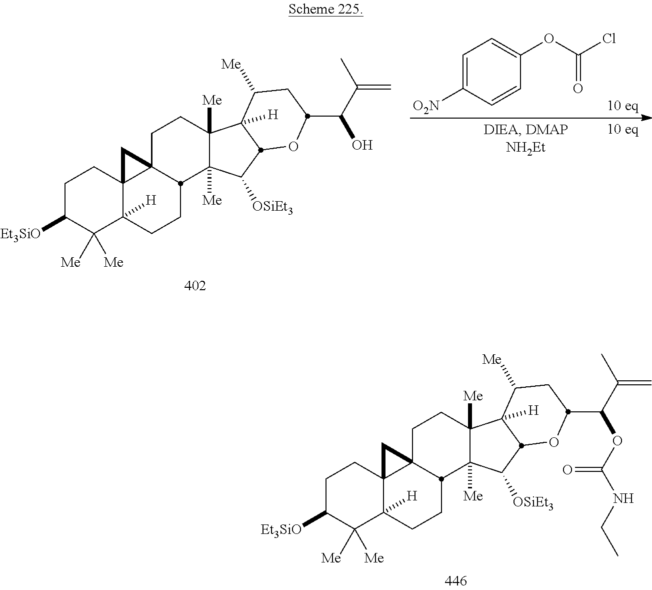

Procedure:

4-Nitrophenyl chloroformate (0.86 g, 4.3 mmol) was added to DIEA (0.275 g, 2.15 mmol), DMAP (0.52 g, 4.3 mmol) and alcohol 402 (300 mg, 0.43 mmol) in dry CH₂Cl₂ (2 mL) under N₂ and allowed to stir for 12 hours. EtNH₂.HCl (175 mg, 2.15 mmol) was added. Then the mixture was stirred at room temperature for another 2 hours. TLC showed the reaction was completed. Water (30 mL) and CH₂Cl₂ (30 mL) was added. The phases were separated. The aqueous phase was extracted with DCM (15 mL×3), dried over Na₂SO₄. The solvent was removed in vacuo to give carbamate 446 which was purified by chromatography (170 mg, 52%).

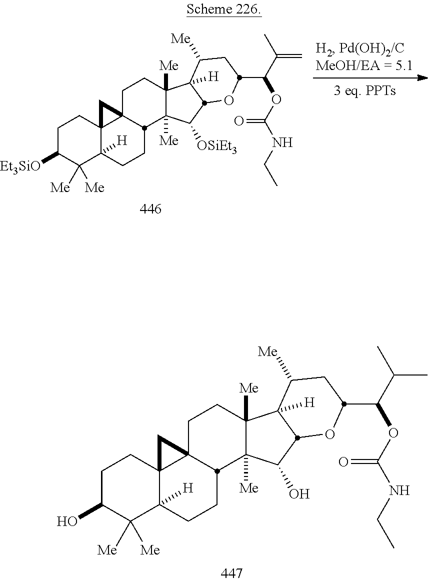

Procedure:

A solution of silyl ether 446 (170 mg, 0.22 mmol) in MeOH (10 mL) and EA (2 mL) was treated with 20% Pd(OH)₂ on carbon (wet) (34 mg). The reaction mixture was stirred under H₂ (1 atm) at room temperature for 30 min. TLC showed that the reaction was finished. Then the mixture was filtered and the filtrate was concentrated in vacuo to give a residue which was dissolved in DCM (2 mL) and MeOH (2 mL) was added PPTS (166 mg, 0.661 mmol,). Then the mixture was stirred at room temperature for 30 minutes. TLC showed the reaction was completed. Solvent was removed in vacuo to give diol 447 which was purified by chromatography (65 mg, 54%).

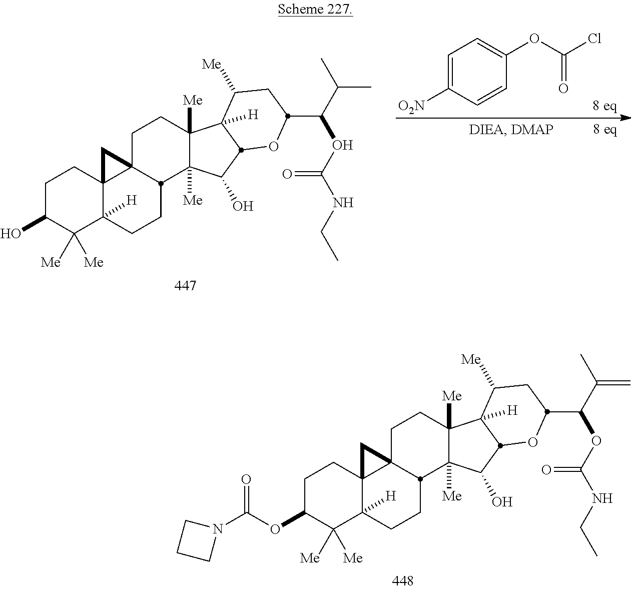

Procedure:

4-Nitrophenyl chloroformate (38 mg, 0.19 mmol) was added to DIEA (16 mg, 0.12 mmol), DMAP (23 mg, 0.19 mmol) and alcohol 447 (13 mg, 0.024 mmol) in dry CH₂Cl₂ (1 mL) under N₂ and allowed to stir for 2 hours. Azetidine hydrochloride (7 mg, 0.071 mmol) was added. Then the mixture was stirred at room temperature for another 2 hours. TLC showed the reaction was completed. Water (30 mL) and CH₂Cl₂ (30 mL) was added. The layers were separated and the aqueous phase was extracted with DCM (15 mL×3). The combined organic phase was dried over Na₂SO₄ and the solvent was removed in vacuo to give carbamate 448 which was purified by preparative HPLC (5.65 mg, 50%). LCMS (m/z): [M+H]⁺ 629

Using the same method, the following products were obtained:

TABLE 53
Compounds

| | |
|---|---|
| 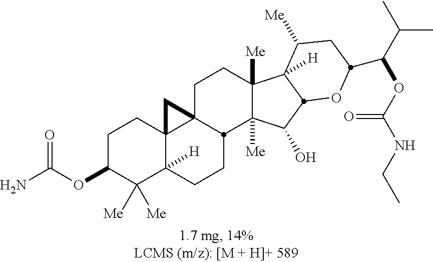 | 449 |
| 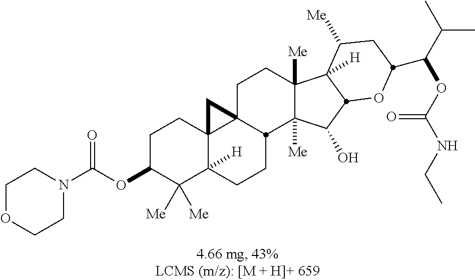 | 450 |
| 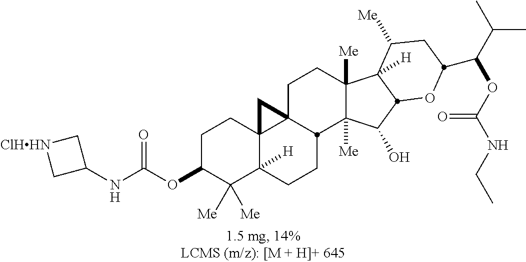 | 451 |
| 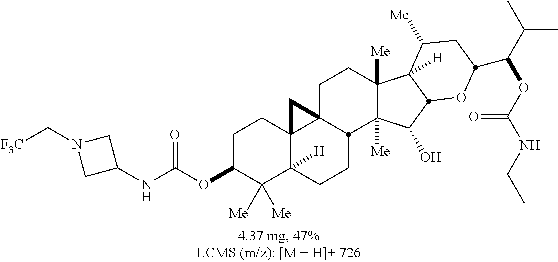 | 452 |

### Example 18

TABLE 54
Exemplary deuterated compounds

| | |
|---|---|
| 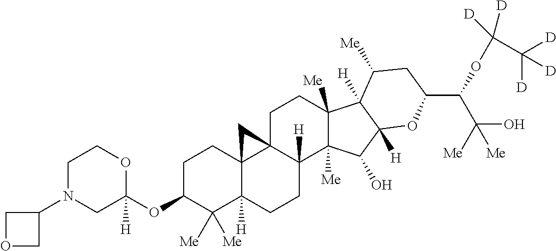 | 453 |
| 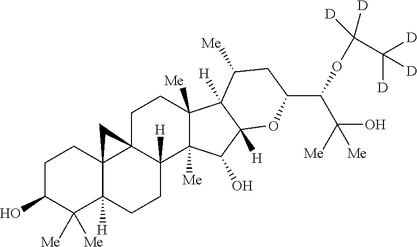 | 454 |

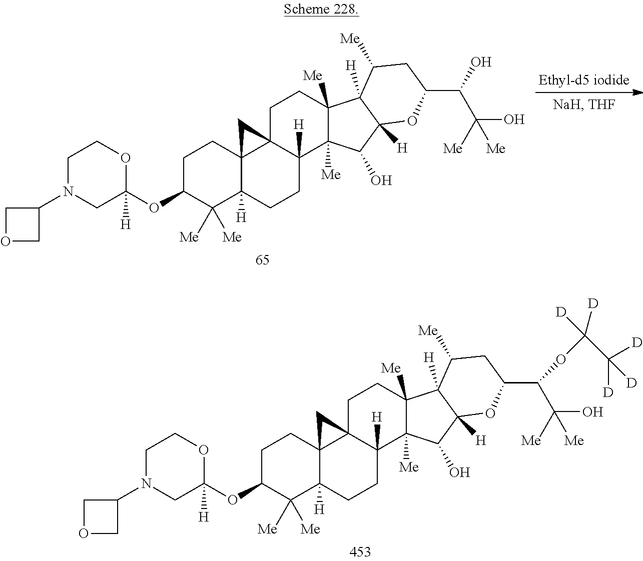

Procedure:

Compound 65 (40 mg) and NaH (10.6 mg, 56-63% dispersion in oil) were dissolved in THF (2 mL) and stirred at rt for 30 min under nitrogen. Ethyl-d5 iodide (35 n1) in THF (0.5 mL) was added and the solution was stirred for 2 days at rt. The solution was diluted in CH₂Cl₂ (15 mL) and washed with aq. 1 M HCl and the organic layer was then removed in vacuo. The residue was purified by C18 chromatography (20-70% ACN/H₂O (0.1% HCO₂H)) to give compound 453 [m/z=665 (M⁺+H)].

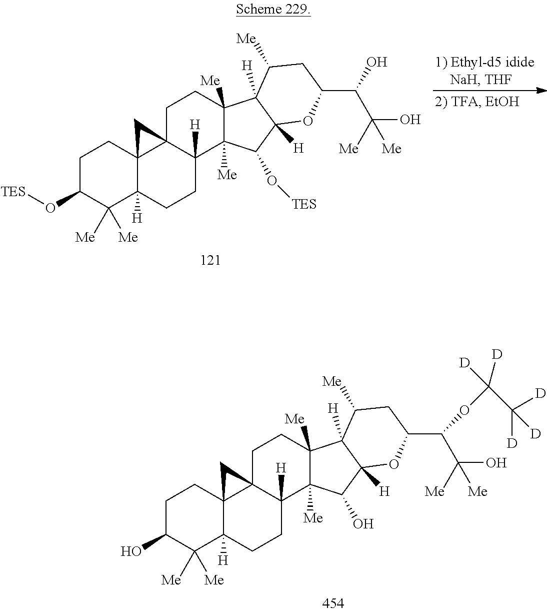

Procedure:

Compound 121 (22 mg) and NaH (5.9 mg, 56-63% dispersion in oil) were dissolved in THF (2 mL) and stirred at rt for 30 min under nitrogen. Ethyl-d5 iodide (39 n1) in THF (0.5 mL) was added and the solution was stirred for 2 days at rt. The solution was diluted in CH₂Cl₂ (15 mL) and washed with aq. 1 M HCl (5 mL) and the organic layer was then removed in vacuo. The residue was dissolved in EtOH (10 mL) and TFA (10 mL) was added and the solvent was then removed in vacuo. The residue was purified by C18 chromatography (30-80% ACN/H₂O (0.1% HCO₂H)) to give compound 454 [m/z=546 (M⁺+Na)].

The compounds of this invention may be prepared or isolated in general by synthetic and/or semisynthetic methods disclosed above and herein, and by synthetic and/or semisynthetic methods known to those of skill in the chemical arts. Where a particular protecting group, leaving group, or transformation condition is depicted above, one of ordinary skill in the art will appreciate that other protecting groups, leaving groups, and transformation conditions are also suitable and are contemplated. Furthermore, for each of the aforementioned Schemes, it will be readily apparent to one of ordinary skill in the art that a variety of suitable reagents and reaction conditions may be employed to synthesize a variety of compounds, for instance, the compounds depicted in Table 55, below.

### Example 19

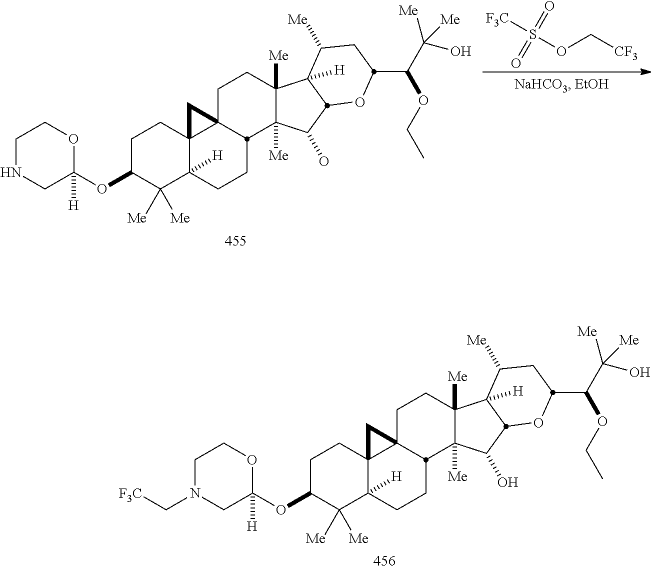

A mixture of 455 (20 mg, 0.033 mmol) in EtOH (5 mL) was added saturated NaHCO₃ solution (5 mL) and trifluoro-methanesulfonic acid 2,2,2-trifluoro-ethyl ester (15.39 mg, 0.066 mmol). The mixture was stirred at 80° C. overnight. The reaction mixture was cooled to room temperature and concentrated to remove EtOH. The aqueous layer extracted with CH₂Cl₂ (3×10 mL). The combined organic phase was dried over Na₂SO₄ and concentrated under reduced pressure to give 456 which was purified by preparative HPLC (15.92 mg, 70%). LCMS (m/z): [M+H]⁺ 686

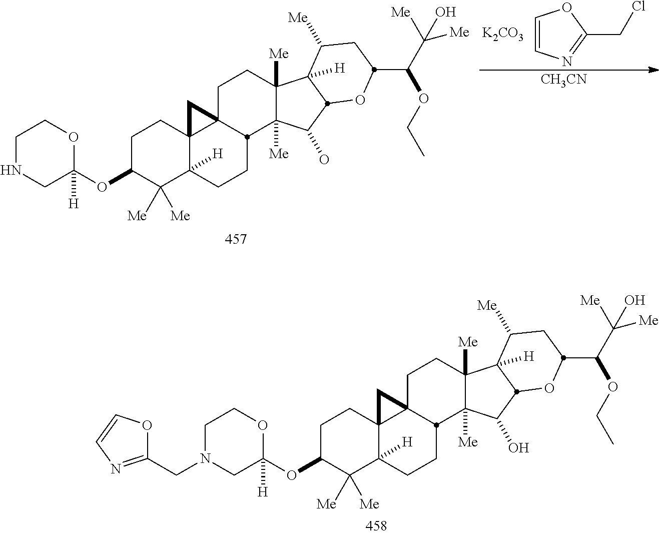

A mixture of compound 457 (15 mg, 0.025 mmol), 2-(chloromethyl)oxazole (2.92 mg, 0.025 mmol) and K₂CO₃ (6.87 mg, 0.050 mmol) in CH₃CN (1 mL) was stirred at 80° C. for 16 hours. LCMS showed that compound 1 was consumed completely. The mixture was purified by prep.HPLC to give pure compound 458 (5.62 mg). LCMS (m/z): [M+H]⁺ 685

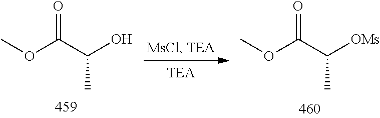

To a solution of compound 459 (300 mg, 2.88 mmol) and Et₃N (0.6 mL, 4.32 mmol) in dry DCM (10 mL) was added dropwise MsCl (264 umL, 3.46 mmol) in an ice water bath. Then the mixture was stirred at room temperature for 1 hour. TLC showed the reaction was completed. Water (30 mL) was added. Then the aqueous layer was extracted with DCM (2×15 mL) and dried over Na₂SO₄. The solvent was removed in vacuo to give compound 460 (505 mg, 96.4%).

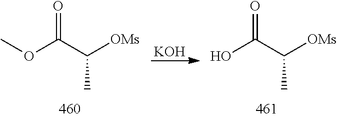

KOH (788 mg, 11.5 mmol) was dissolved in 6.5 mL of H₂O, compound 460 (1.8 g, 9.6 mmol) was added. The mixture was stirred for 4 h at rt. The reaction was treated with con.HCl. The pH was adjusted about 1. Then the mixture was concentrated in vacuo to dryness. The residue was treated with 20 mL of MeOH, and stirred for 30 min. The mixture was filtered and the filtrate was concentrated in vacuo to give compound 461 (1.6 g, 97.0%).

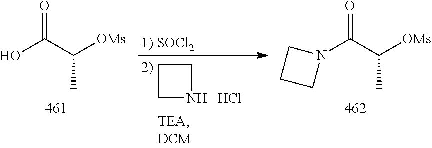

A mixture of compound 461 (300 mg, 1.74 mmol) and 0.5 mL of SOCl₂ was heated to refluxed for 3 h. The solvent was evaporated in vacuo to dryness. The residue was dissolved in 6 ml, of dry DCM. Then TEA (726 umL, 5.22 mmol) was added at 0° C., followed by azetidine HCl salt (180 mg, 1.92 mmol.). Then the mixture was stirred for 2 h at rt. The mixture was diluted with 10 mL of DCM, washed with 1 N HCl, sat aq NaHCO₃, dried over Na₂SO₄ and evaporated to give compound 462 (210 mg, 58.3%).

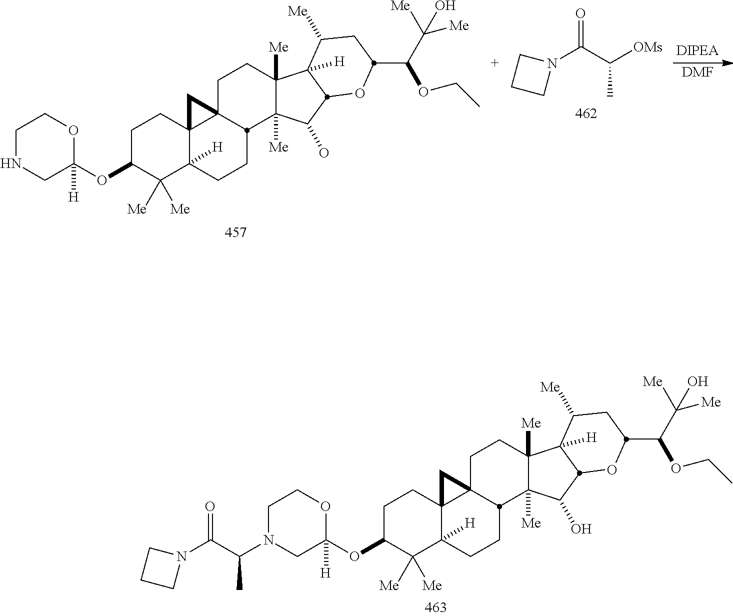

To a solution of 457 (50 mg, 0.083 mmol) and compound 462 (86 mg, 0.415 mmol) in DMF (1 mL) was added DIPEA (107 mg, 0.83). The reaction mixture was stirred at 80° C. for 18 h. LC-MS showed the reaction was completed. The mixture was purified by prep HPLC to give 463 (17.5 mg, 29.5%). LCMS (m/z): [M+H]⁺ 715

The same method was used to provide the following compounds:

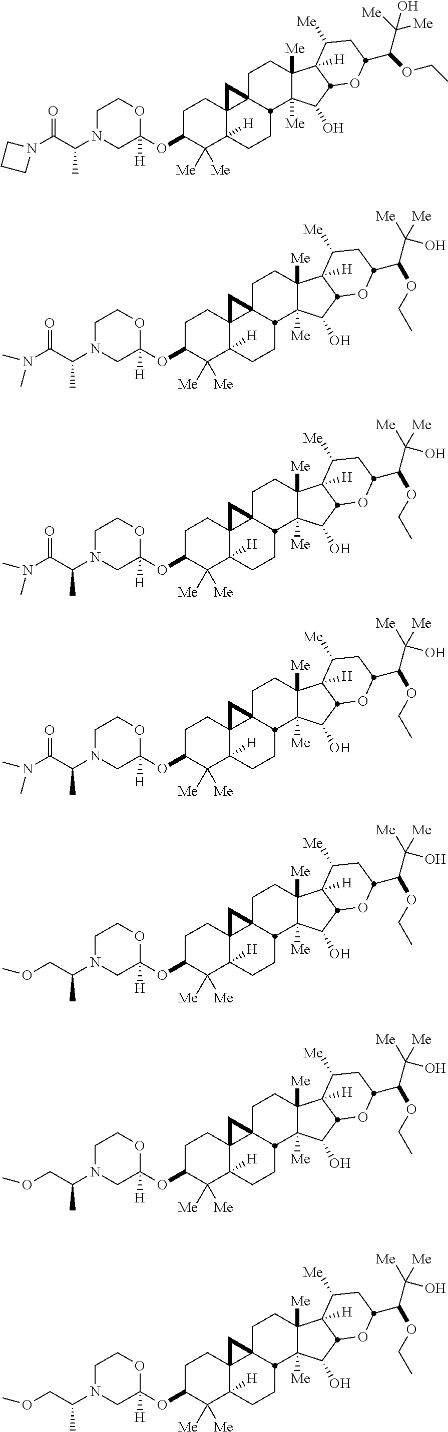

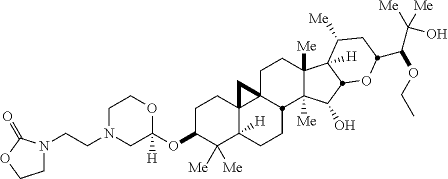

### Example 20

### Biological Assays: Aβ-42, Aβ-40, and Aβ-38

Assays were conducted to determine the ability of a Compound of Formula I to modulate Aβ-40, Aβ-40, and Aβ-38.

Procedure:

μELISA Plates:

Human (6E10) Ab 3-PLEX elisa kits were purchased from Meso Scale Discovery Labs, 9328 Gaither Road, Gaithersburg, Md. 20877 (Catalog Number K15148E-3). Plates with capture antibodies were blocked for 1-2 hours at room temperature with 150 μL of the manufactures blocking reagent.

Conditioned Media:

Cultured 2B7 cells in 96 well plate with 250 uL of media per well until confluent;

Prepared serial dilutions of compounds in DMSO at 100× the final desired concentration;

Washed wells with 2B7 cells 1× with 250 uL of media;

Diluted DMSO stocks 1:100 into media:

Added 250 μL of media containing compounds (1% DMSO) to wells with 2B7 cells for 5 hours at 37° C.

Elisa Sample Prep:

Diluted conditioned media: 1 part media with 1% DMSO and 1 part blocking buffer;

150 μL of the 250 μL of conditioned media were used.

Standard Curve Sample Prep:

Prepared per manufacturer's protocol (see above)

Seven point standard curve samples were prepared that contained Aβ-42, Aβ-40, and Aβ-38. The highest concentration of Aβ-42 and Aβ-38 was 3,000 pg/mL and the highest concentration of Aβ-40 was 10,000 pg/mL. Subsequent serial dilutions were 1:3 and the final composition of each sample was 1 part blocking buffer and 1 part cell medium containing 1% DMSO.

Overnight Sample Incubation:

Blocked plates are washed 5× with MSD wash buffer with a plate washer

25 uL of detection antibody and blocker G reagent in MSD blocking solution is added

25 uL of samples (1 part conditioned media containing 1% DMSO and 1 part MSD blocking buffer) are then added.

plates are incubated overnight at 4 degrees C. or 2 hours at room temp

Final Wash and Readout:

Wash wells 5× with MSD wash buffer

Added 150 μL 2×MSD read buffer

Read with MSD imager.

Buffers:

All reagents are in kit

Data Analysis

A-Beta peptide levels for each peptide are calculated from the standard curve using the MSD software provided with the MSD 2400 Imager. Percent vehicle values for each compound dosage were then calculated and fit to a 4 parameter curve generating IC50 values.

Cell Viability:

To the remaining 100 uL of conditioned media in the tissue culture plate is added 100 uL of CellTiter-Glo reagent from Promega. The plate is placed on an orbital rotator operating at 500 rpms for 2 minutes. The plate is left static for 10 minutes and then 150 uL of the lysates are transferred to a white plate and read in a luminometer.

Biological Activity Data (Table 55):

Compounds having an activity designated as “A” provided an IC₅₀≦1000 nM; compounds having an activity designated as “B” provided an IC₅₀ of 1000-10,000 nM; and compounds having an activity designated as “C” provided an IC₅₀ of >10,000 nM. In certain instances a compound was tested more than once and exhibited more than one IC₅₀ value. In such instances, if all IC₅₀ values fall within the same range then that range is indicated using the appropriate “A,” “B,” or “C” designation set forth above. In instances wherein values fall within two different ranges, the designations “A-B” or “B-C” are used.

TABLE 55

| | Aβ- | Aβ- | Aβ- |
|---|---|---|---|
| | 38 | 40 | 42 |
| 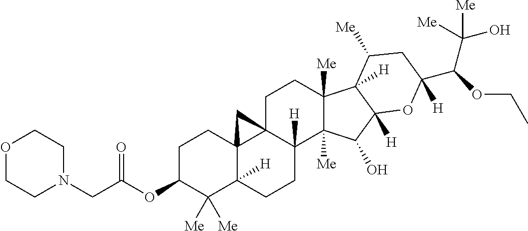 | A | B | A |
| 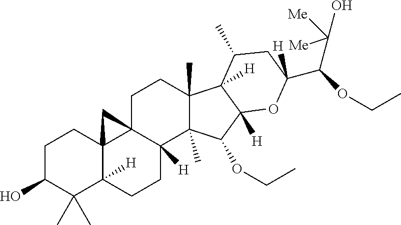 | B | B | B |
| 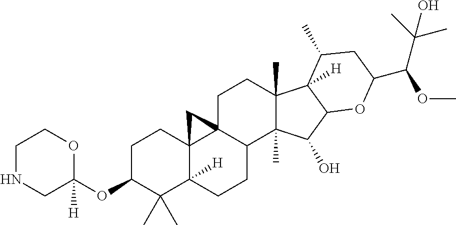 | A | B | A |
| 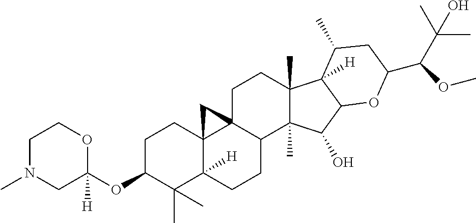 | B | B | B |
| 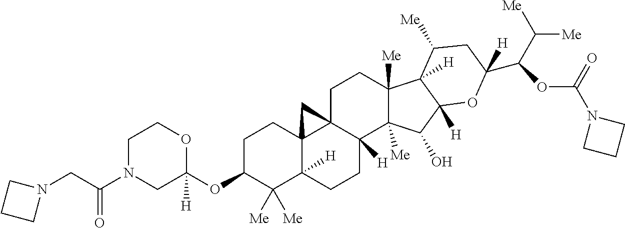 | A | B | A |
| 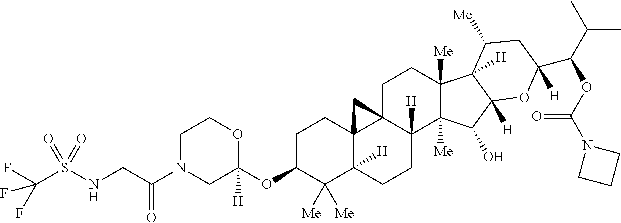 | A | — | B |
| 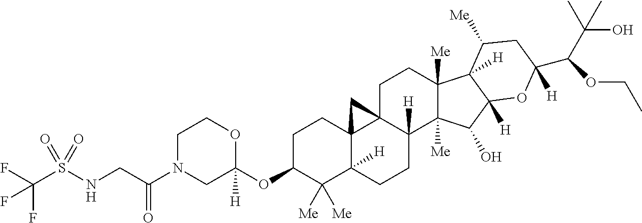 | A | B | A |
| 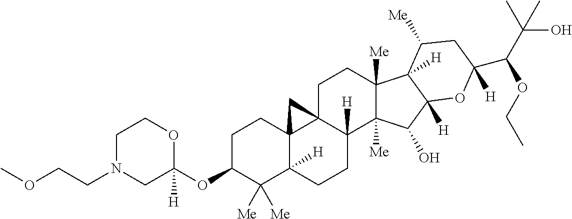 | — | — | — |
| 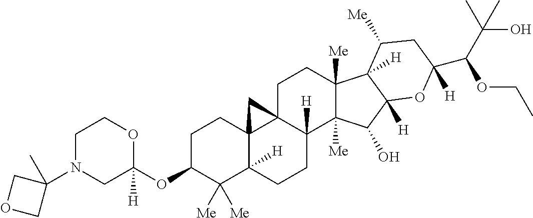 | A | B | A |
| 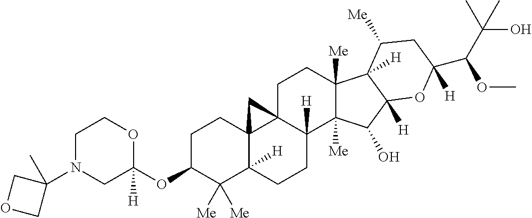 | B | — | B |
| 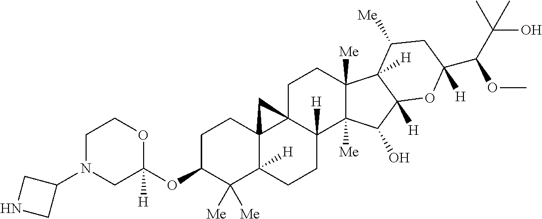 | A | B | A |
| 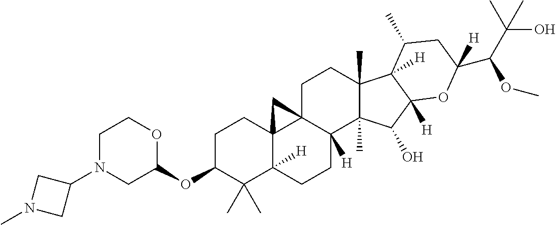 | A | B | A |
| 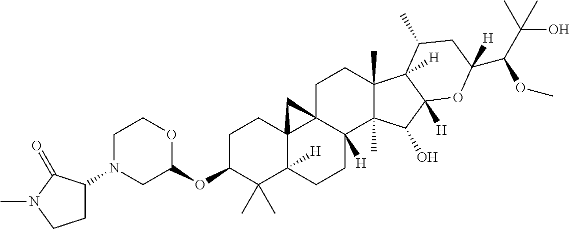 | A | C | A |
| 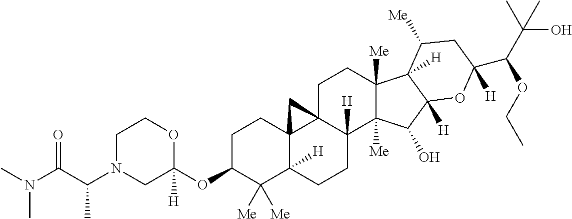 | A | — | A |
| 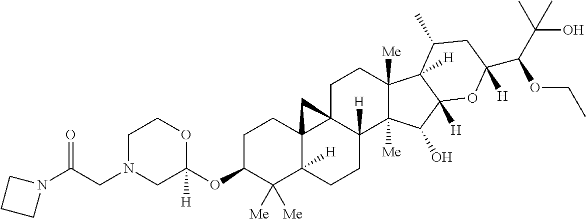 | A | B | A |
| 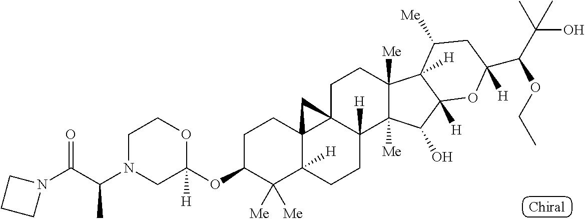 | B | C | A |
| 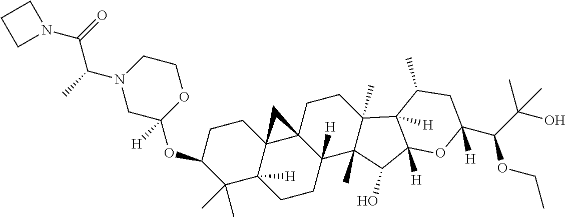 | A | — | A |
| 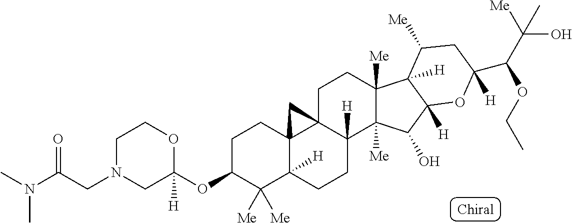 | A | B | A |
| 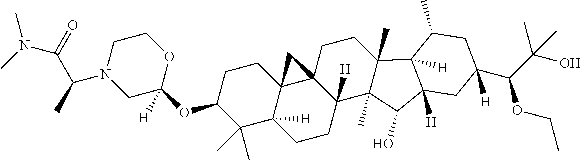 | — | — | A |
| 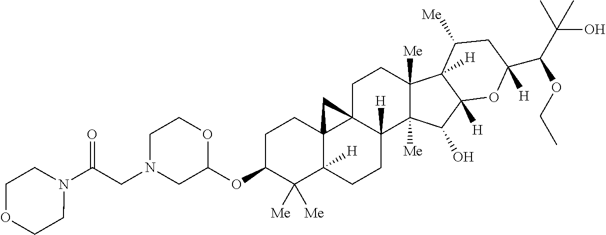 | A | C | A |
| 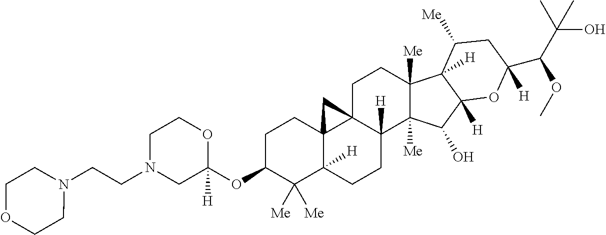 | A | B | A |
| 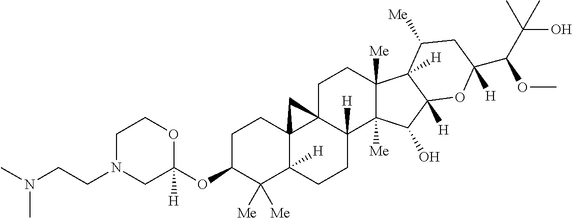 | B | C | A |
| 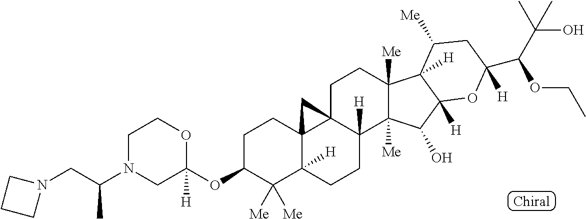 | A | B | A |
| 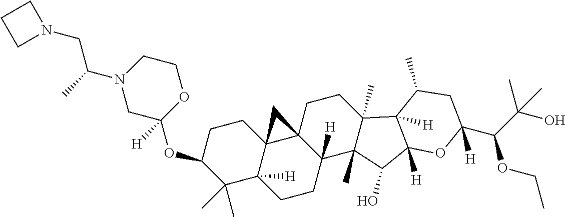 | A | — | A |
| 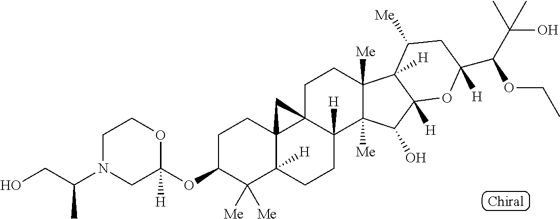 | A | B | A |
| 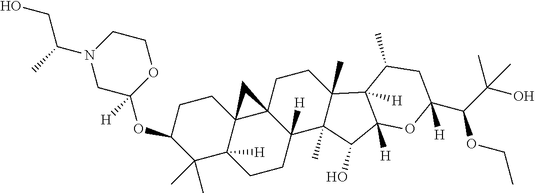 | B | B | A |
| 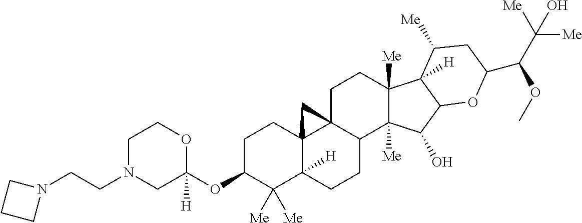 | — | — | A |
| 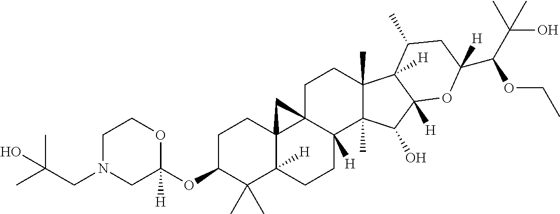 | A | B | A |
| 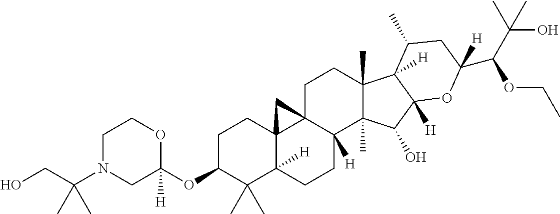 | A | B | A |
| 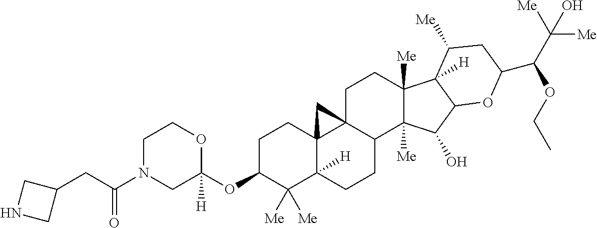 | A | B | A |
| 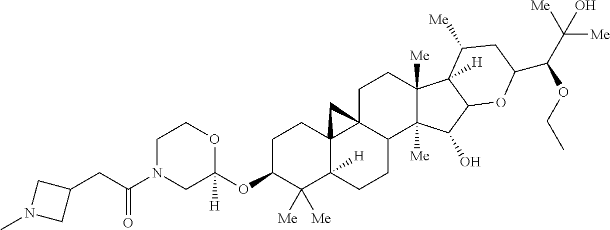 | A | B | A |
| 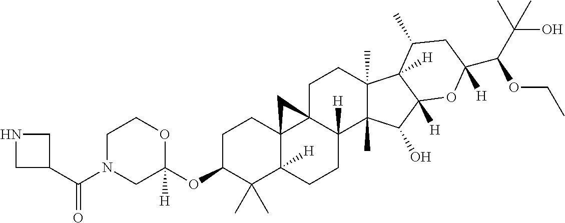 | A | B | A |
| 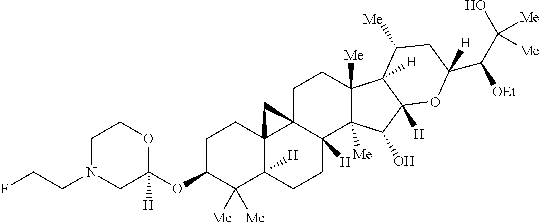 | A | B | A |
| 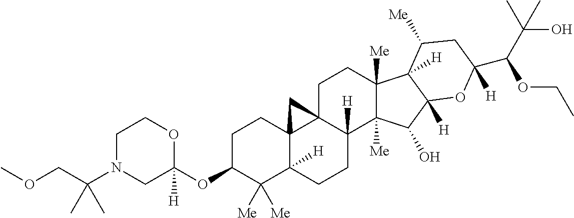 | A | C | A |
| 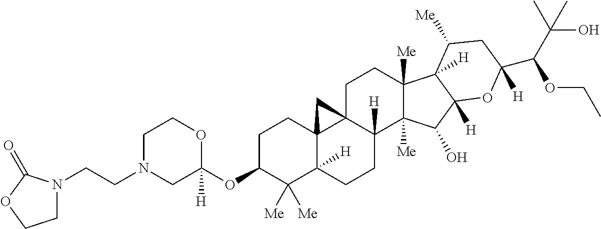 | A | B | A |
| 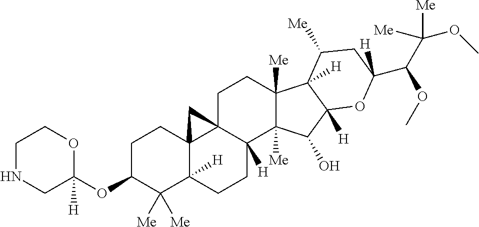 | A | — | A |
| 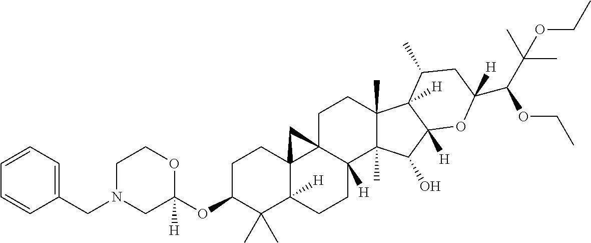 | — | — | — |
| 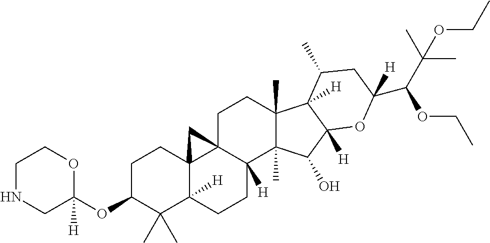 | B | — | B |
| 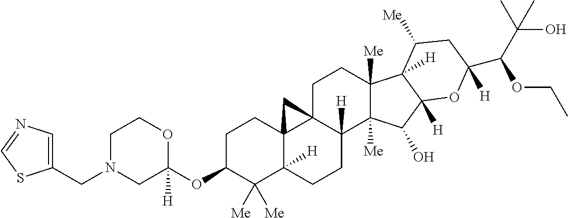 | B | — | A |
| 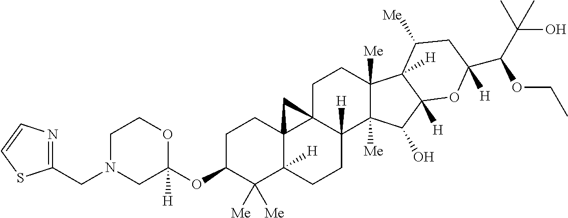 | B | — | A |
| 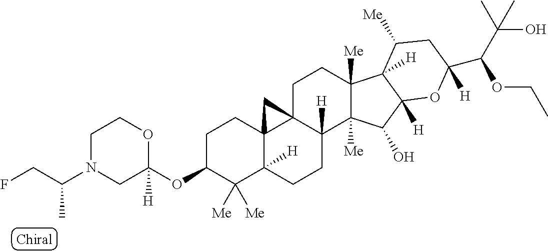 | B | C | B |
| 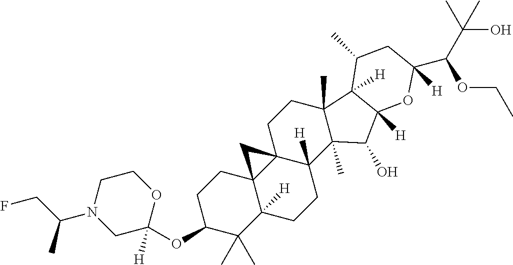 | B | C | B |
| 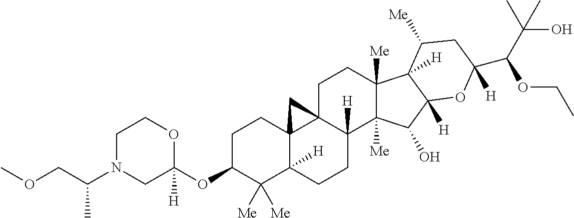 | B | C | A |
| 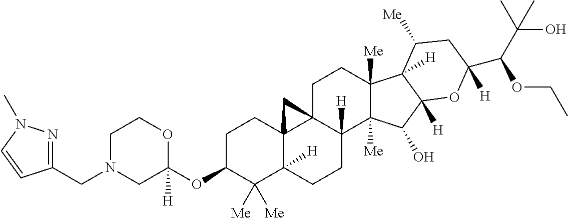 | B | C | A |
| 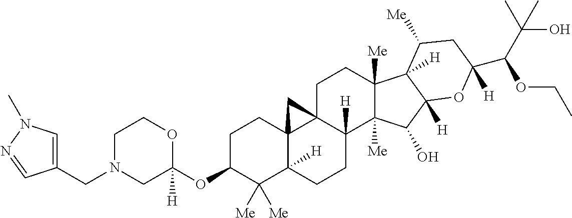 | B | — | A |
| 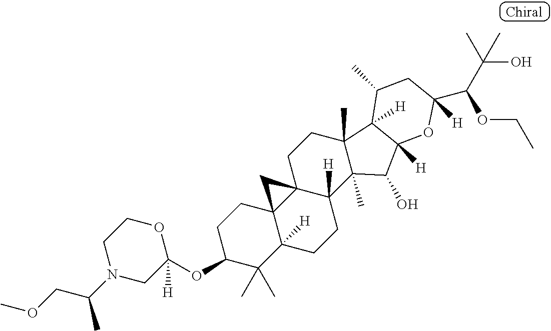 | B | C | A |
| 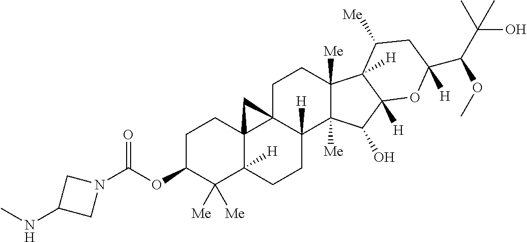 | B | — | A |
| 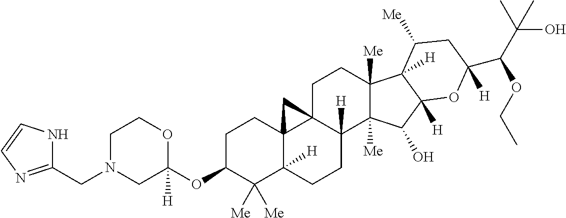 | A | B | A |
| 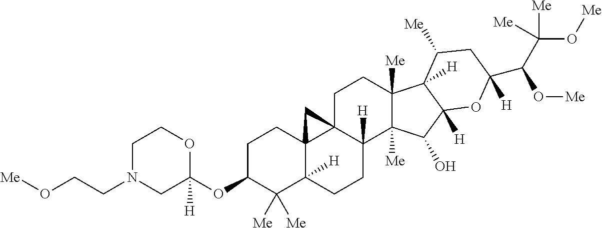 | B | C | A |
| 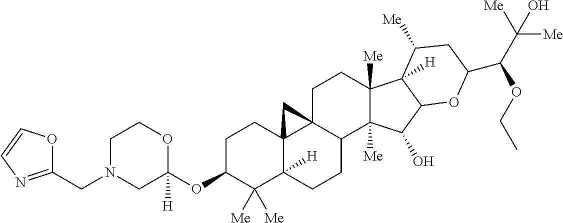 | B | — | A |
| 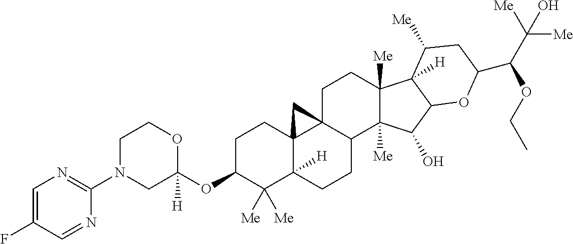 | B | — | B |
| 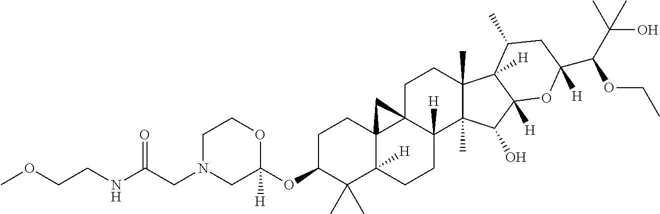 | B | B | A |
| 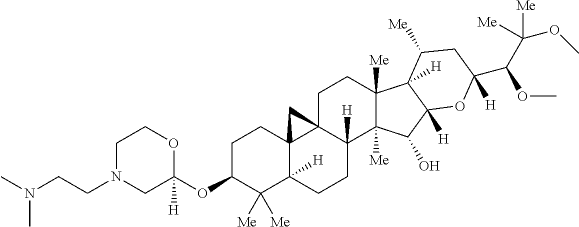 | A | B | A |
| 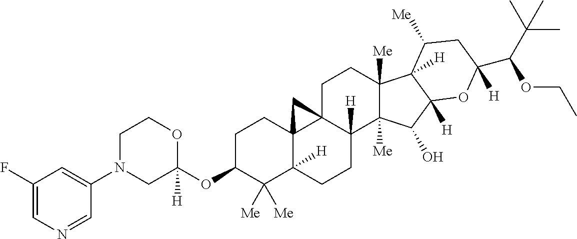 | B | C | B |
| 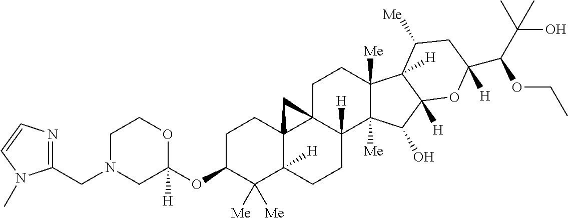 | B | — | A |
| 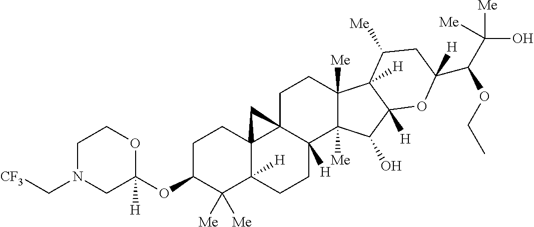 | B | — | B |
| 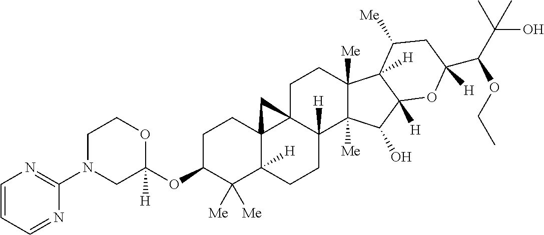 | B | — | B |
| 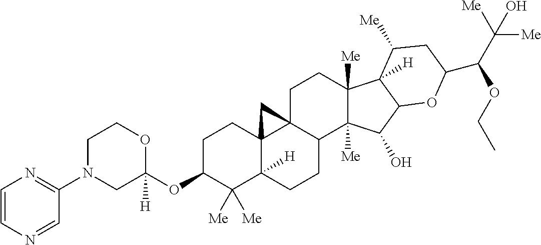 | A | C | A |
| 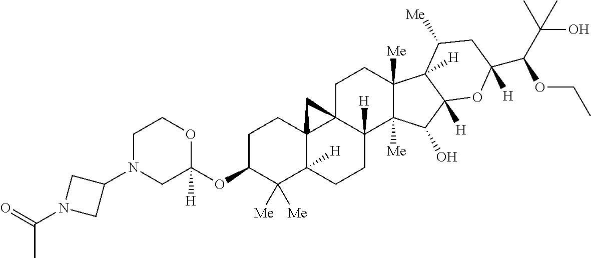 | B | B | A |
| 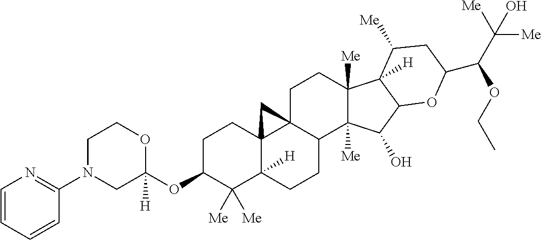 | B | B | B |
| 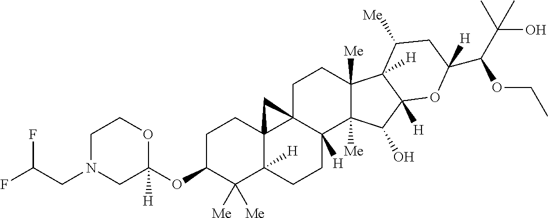 | B | — | A |
| 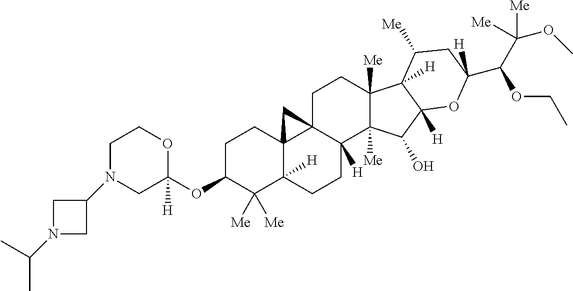 | A | B | A |
| 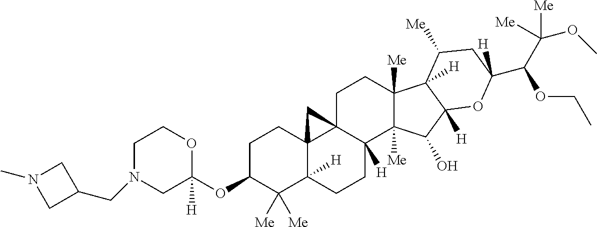 | A | B | A |
| 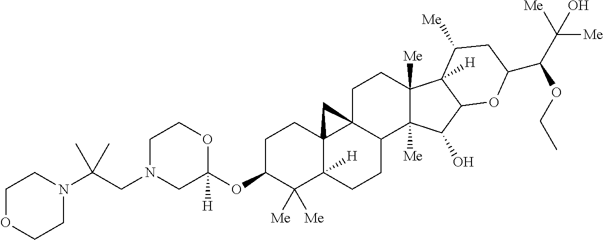 | A | B | A |
| 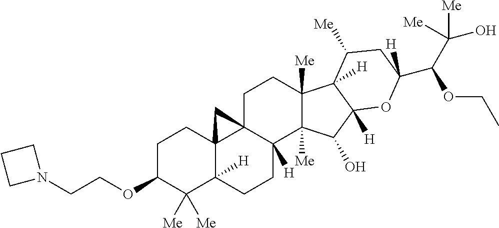 | — | — | — |
| 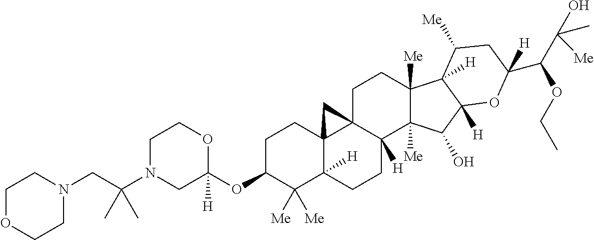 | A | B | A |
| 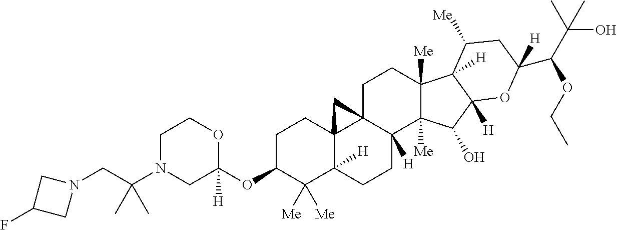 | A | B | A |
| 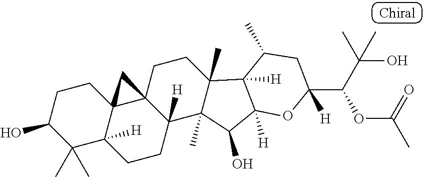 | — | B | B |
| 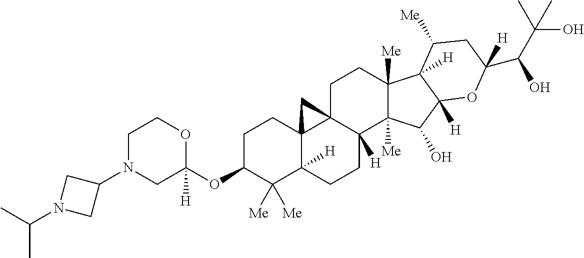 | — | — | — |
| 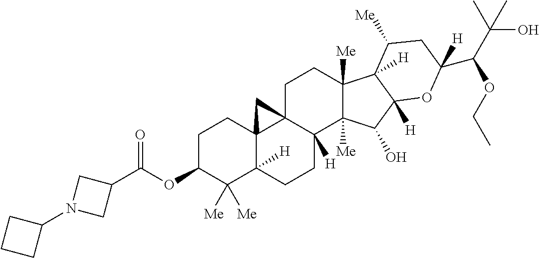 | A | B | A |
| 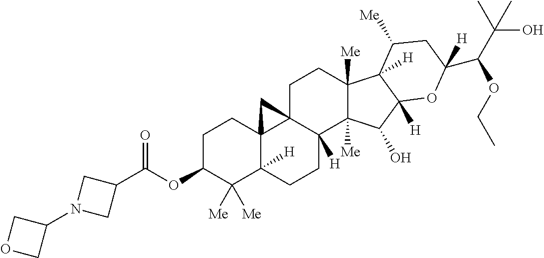 | B | — | A |
| 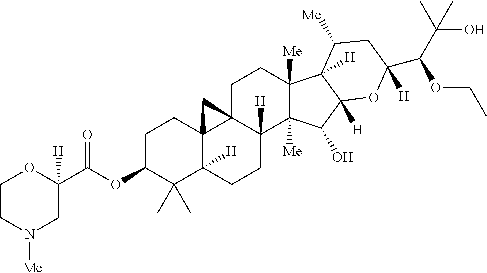 | B | — | A |
| 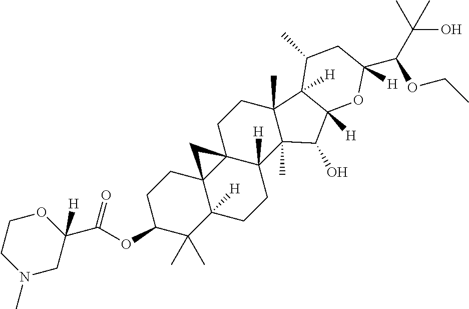 | B | — | A |
| 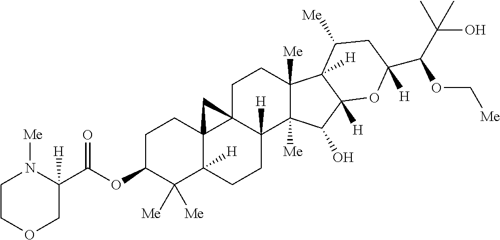 | A | C | A |
| 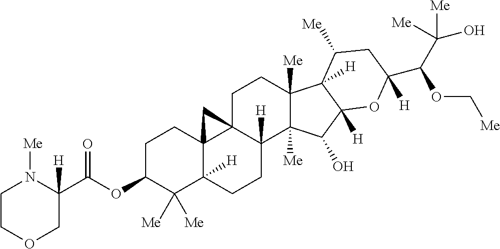 | A | B | A |
| 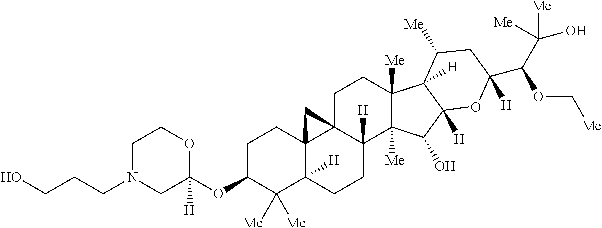 | B | B | A |
| 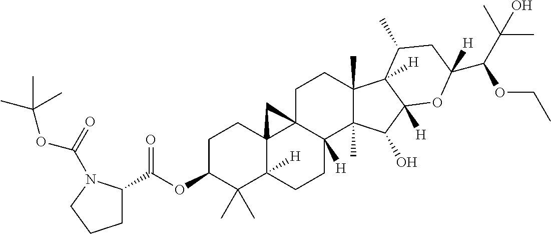 | B | — | B |
| 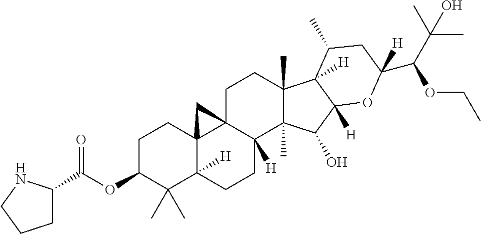 | A | B | A |
| 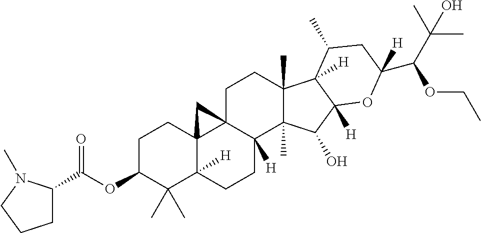 | A | B | A |
| 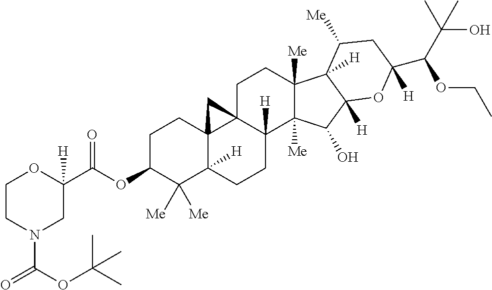 | B | — | B |
| 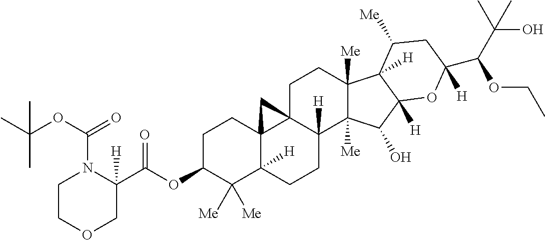 | B | — | B |
| 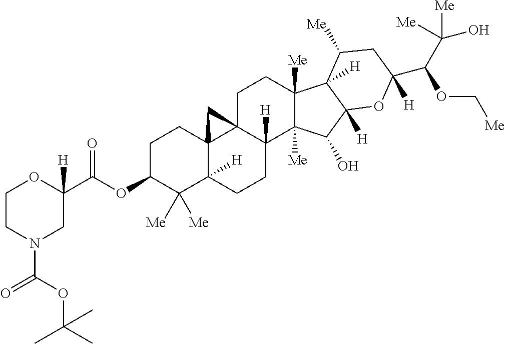 | A | — | A |
| 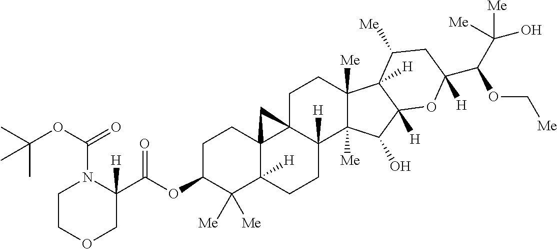 | B | — | B |
| 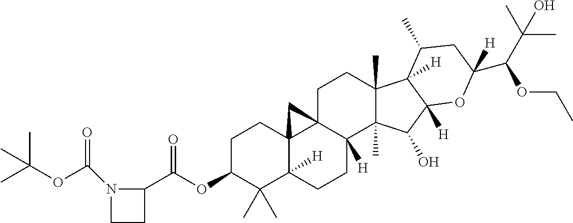 | B | B | A |
| 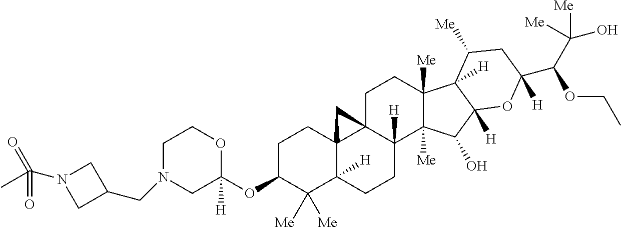 | A | — | A |
| 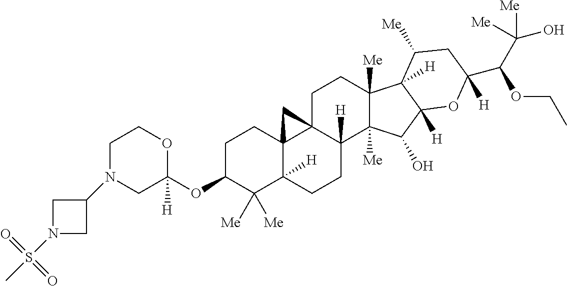 | A | — | A |
| 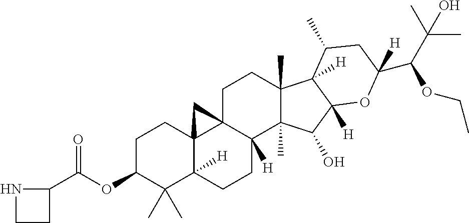 | A | B | A |
| 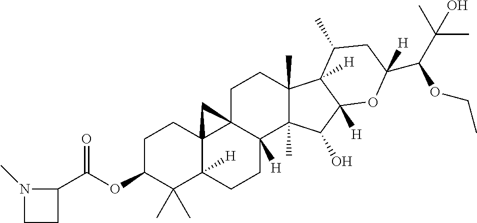 | B | C | A |
| 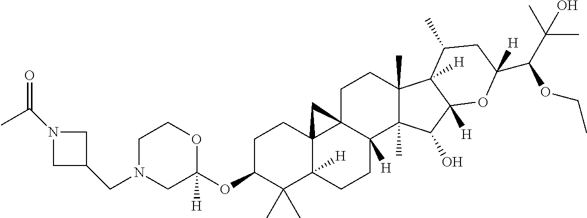 | B | B | A |
| 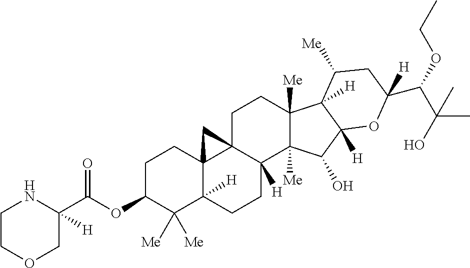 | A | B | A |
| 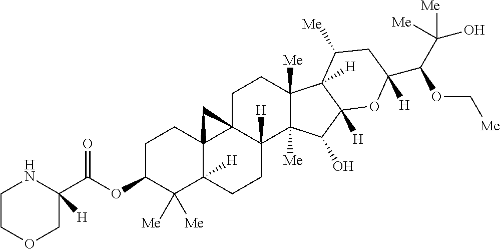 | A | B | A |
| 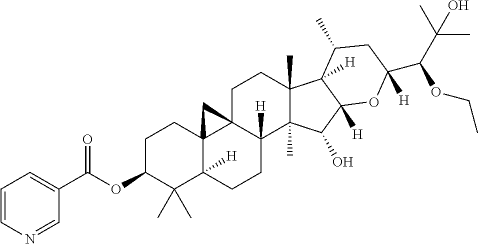 | A | C | A |
| 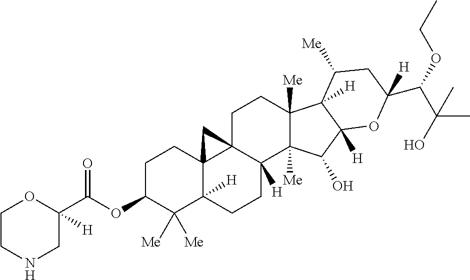 | A | B | A |
| 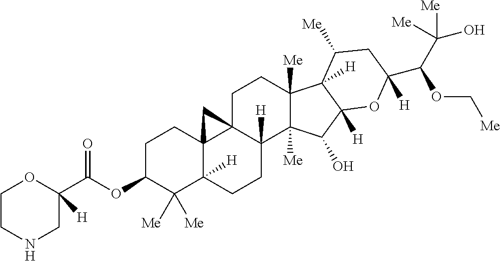 | A | — | A |
| 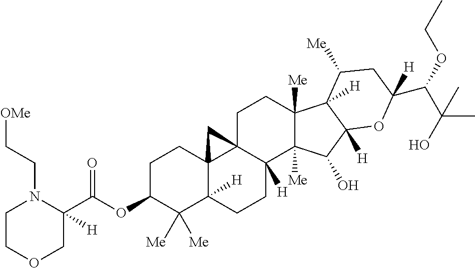 | A | B | A |
| 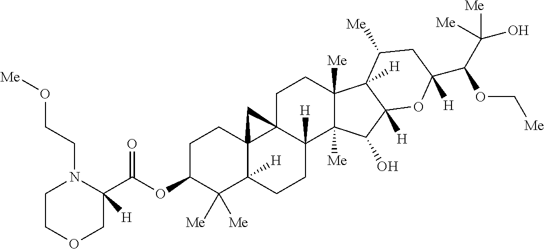 | B | — | A |
| 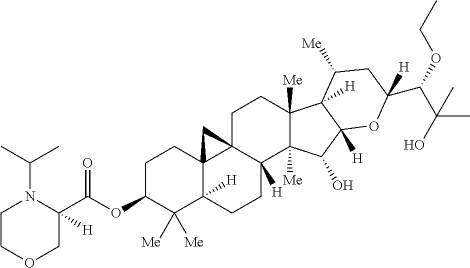 | B | — | A |
| 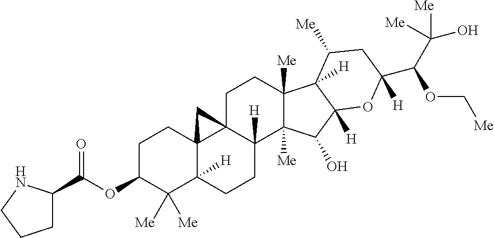 | A | B | A |
| 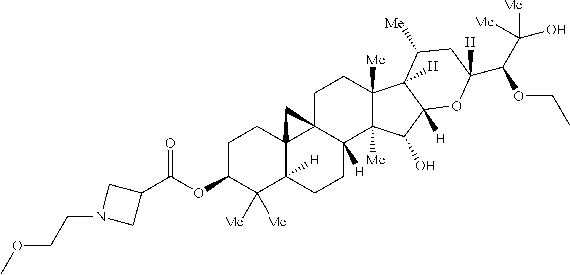 | A | B | A |
| 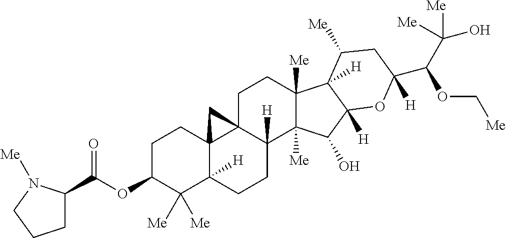 | A | B | A |
| 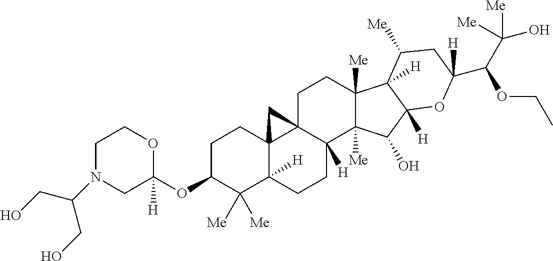 | A | — | A |
| 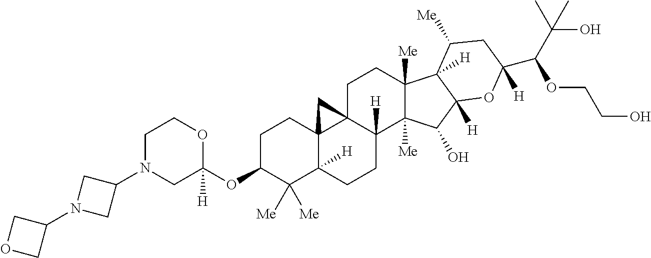 | B | — | B |
| 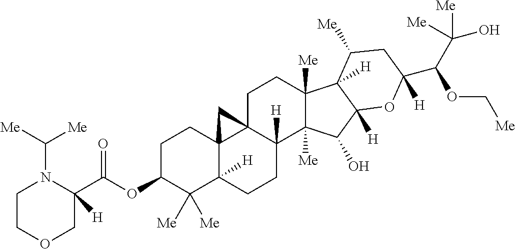 | B | C | A |
| 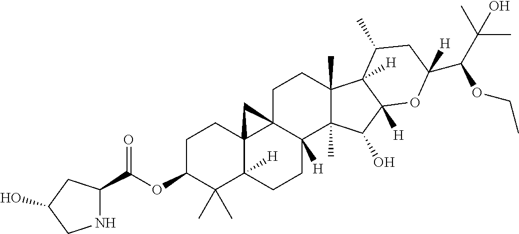 | A | B | A |
| 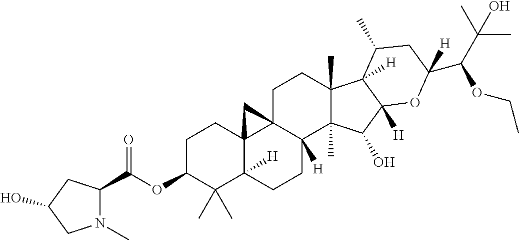 | A | B | A |
| 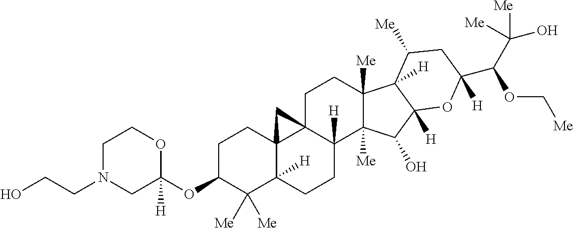 | A | B | A |
| 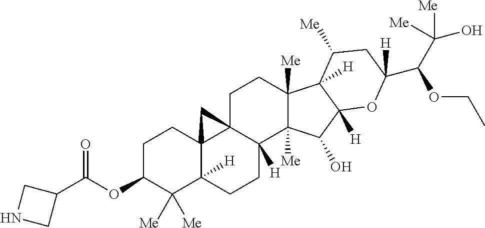 | A | B | A |
| 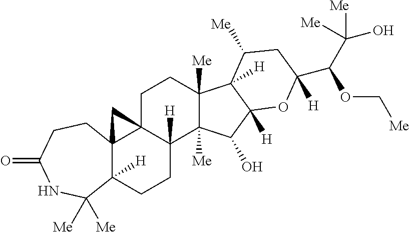 | — | — | — |
| 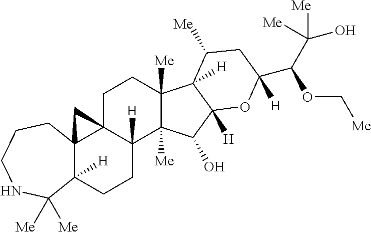 | — | — | — |
| 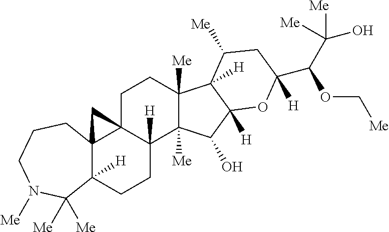 | — | — | — |
| 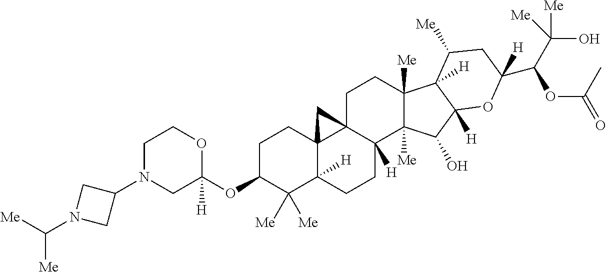 | — | — | — |
| 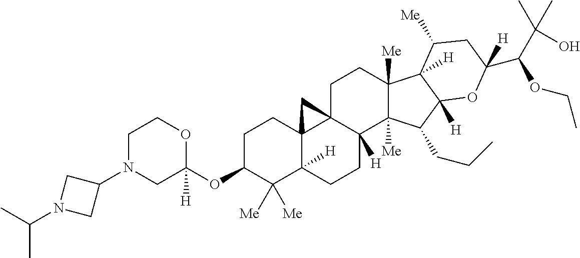 | — | — | — |
| 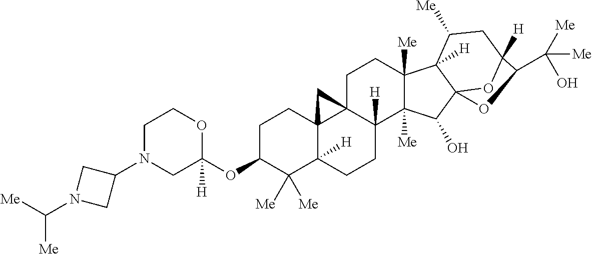 | — | — | — |
| 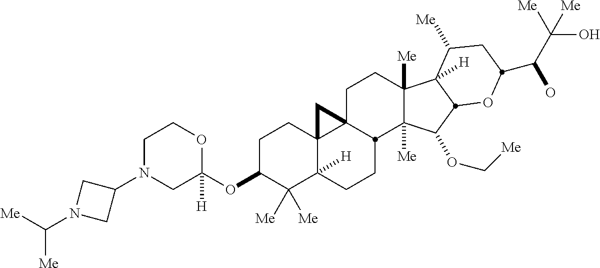 | — | — | — |
| 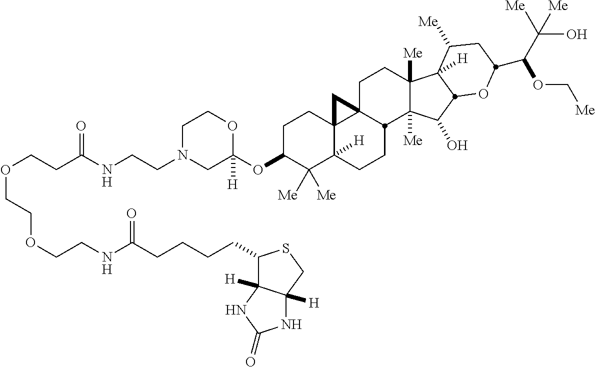 | B | C | B |
| 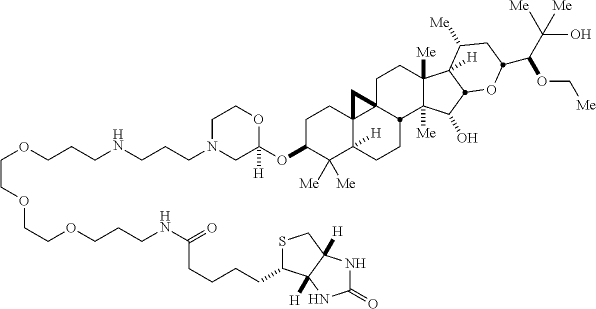 | A | B | A |
| 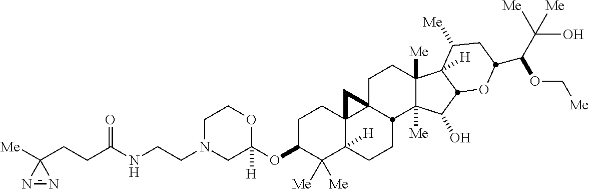 | A | C | A |
| 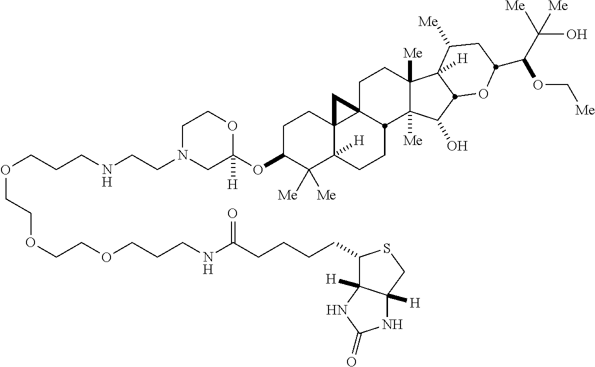 | A | B | A |
| 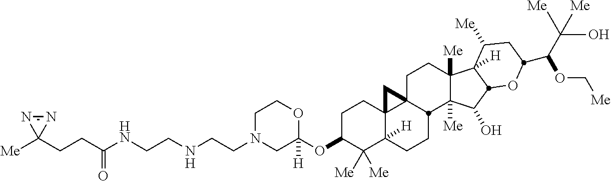 | B | — | A |
| 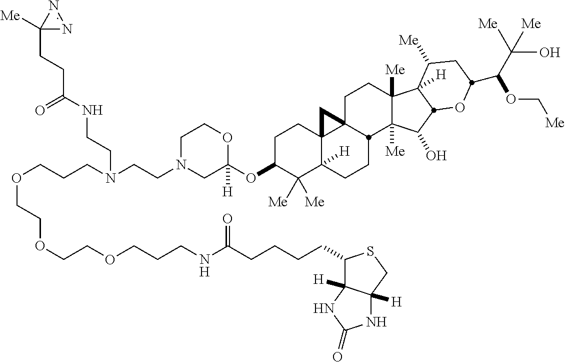 | — | B | A |
| 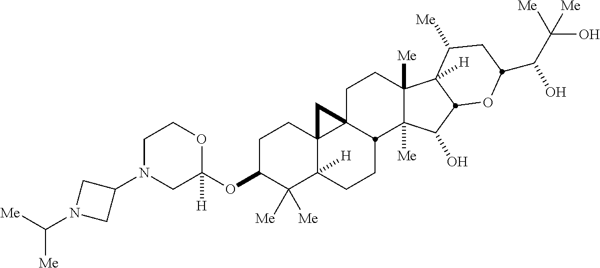 | — | — | — |
| 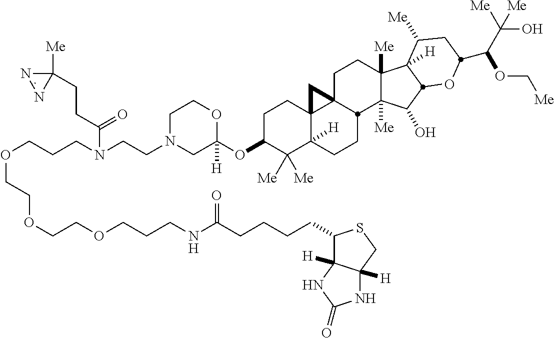 | — | — | — |
| 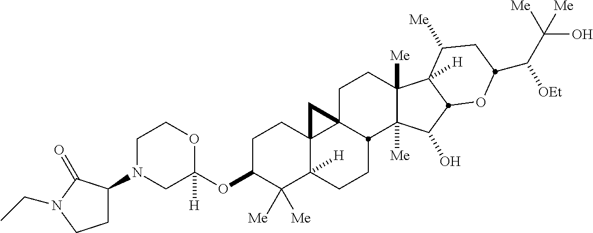 | — | — | — |

1. A compound of formula I: 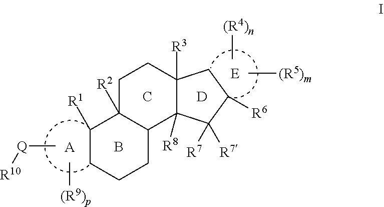
or a pharmaceutically acceptable salt thereof, wherein:
Ring A is a 4-7 membered saturated or partially unsaturated ring having 0-2 heteroatoms independently selected from nitrogen, oxygen, or sulfur;
each of Ring B, Ring C, and Ring D is independently saturated, partially unsaturated or aromatic, or a deuterated derivative thereof;
Ring E is a 4-7 membered saturated, partially unsaturated, or aromatic ring having 0-2 heteroatoms independently selected from nitrogen, oxygen, or sulfur;
R¹ and R² are each independently halogen, R, OR, a suitably protected hydroxyl group, SR, a suitably protected thiol group, N(R)₂, or a suitably protected amino group, or R¹ and R² are taken together to form a 3-7 membered saturated or partially unsaturated ring having 0-2 heteroatoms independently selected from nitrogen, oxygen, or sulfur;
each R is independently deuterium, hydrogen, an optionally substituted C₁₋₆ aliphatic group, or an optionally substituted 3-8 membered saturated, partially unsaturated, or aryl ring having 0-4 heteroatoms independently selected from nitrogen, oxygen, or sulfur, wherein:
two R on the same nitrogen atom are optionally taken together with said nitrogen atom to form an optionally substituted 3-8 membered, saturated, partially unsaturated, or aryl ring having 1-4 heteroatoms independently selected from nitrogen, oxygen, or sulfur;
n is 0-4;
R³, R⁴, and R⁸ are each independently selected from halogen, CN, R, OR, a suitably protected hydroxyl group, SR, a suitably protected thiol group, S(O)R, SO₂R, OSO₂R, N(R)₂, a suitably protected amino group, N(R)C(O)R, N(R)C(O)C(O)R, N(R)C(O)N(R)₂, N(R)C(O)OR, C(O)OR, OC(O)R, C(O)N(R)₂, or OC(O)N(R)₂, or:
two R⁴ on the same carbon are optionally taken together to form an optionally substituted 3-8 membered saturated or partially unsaturated spirofused ring having 0-4 heteroatoms independently selected from nitrogen, oxygen, or sulfur, or:
two R⁴ on the same carbon are optionally taken together to form an oxo moiety, an oxime, an optionally substituted hydrazone, an optionally substituted imine, or an optionally substituted C₂₋₆ alkylidene;
m is 0-4;
each R⁵ is independently T-C(R′)₃, T-C(R′)₂C(R″)₃, OR, a suitably protected hydroxyl group, SR, a suitably protected thiol group, S(O)R, SO₂R, OSO₂R, N(R)₂, a suitably protected amino group, N(R)C(O)R, N(R)C(O)C(O)R, N(R)C(O)N(R)₂, N(R)C(O)OR, C(O)OR, OC(O)R, C(O)N(R)₂, or OC(O)N(R)₂, an optionally substituted 3-8 membered saturated, partially unsaturated, or aryl monocyclic ring having 0-4 heteroatoms independently selected from nitrogen, oxygen, or sulfur, an optionally substituted 8-10 membered saturated, partially unsaturated, or aryl bicyclic ring having 0-4 heteroatoms independently selected from nitrogen, oxygen, or sulfur, or:
two R⁵ on the same carbon are optionally taken together to form an oxo moiety, an oxime, an optionally substituted hydrazone, an optionally substituted imine, an optionally substituted C₂₋₆ alkylidene, or an optionally substituted 3-8 membered saturated or partially unsaturated spirocycle having 0-4 heteroatoms independently selected from nitrogen, oxygen, or sulfur;
each T is independently a valence bond or an optionally substituted straight or branched, saturated or unsaturated, C₁₋₆ alkylene chain wherein up to two methylene units of T are optionally and independently replaced by —O—, —N(R)—, —S—, —C(O)—, —S(O)—, or —S(O)₂—;
each R′ and R″ is independently selected from halogen, R, OR, SR, S(O)R, SO₂R, OSO₂R, N(R)₂, N(R)C(O)R, N(R)C(O)C(O)R, N(R)C(O)N(R)₂, N(R)C(O)OR, N(R)S(O)R, N(R)SO₂R, N(R)SO₂OR C(O)OR, OC(O)R, C(O)N(R)₂, OC(O)N(R)₂, or an optionally substituted 3-8 membered saturated, partially unsaturated, or aryl monocyclic ring having 0-4 heteroatoms independently selected from nitrogen, oxygen, or sulfur, or an optionally substituted 8-10 membered saturated, partially unsaturated, or aryl bicyclic ring having 0-4 heteroatoms independently selected from nitrogen, oxygen, or sulfur, or:
two R′ are optionally taken together to form an oxo moiety, an oxime, an optionally substituted hydrazone, an optionally substituted imine, an optionally substituted C₂₋₆ alkylidene, or an optionally substituted 3-8 membered saturated or partially unsaturated ring having 0-4 heteroatoms independently selected from nitrogen, oxygen, or sulfur, or:
two R″ are optionally taken together to form an oxo moiety, an oxime, an optionally substituted hydrazone, an optionally substituted imine, an optionally substituted C₂₋₆ alkylidene, or an optionally substituted 3-8 membered saturated or partially unsaturated ring having 0-4 heteroatoms independently selected from nitrogen, oxygen, or sulfur;
R⁶ is halogen, R, OR, SR, S(O)R, SO₂R, OSO₂R, N(R)₂, N(R)C(O)R, N(R)C(O)C(O)R, N(R)C(O)N(R)₂, N(R)C(O)OR, C(O)OR, OC(O)R, C(O)N(R)₂, or OC(O)N(R)₂, or:
R⁶ and R⁵ are optionally taken together to form an optionally substituted 3-8 membered saturated, partially unsaturated, or aryl ring having 0-4 heteroatoms independently selected from nitrogen, oxygen, or sulfur;
each of R⁷ and R^{7′} is independently selected from halogen, CN, N₃, R, OR, a suitably protected hydroxyl group, SR, a suitably protected thiol group, S(O)R, SO₂R, OSO₂R, N(R)₂, a suitably protected amino group, NRC(O)R, NRC(O)C(O)R, N(R)C(O)N(R)₂, N(R)C(O)OR, C(O)OR, OC(O)R, C(O)N(R)₂, or OC(O)N(R)₂, or:
R⁷ and R^{7′} are taken together to form an oxo moiety, an oxime, an optionally substituted hydrazone, an optionally substituted imine, an optionally substituted C₂₋₆ alkylidene, or an optionally substituted 3-8 membered saturated or partially unsaturated spirocycle having 0-4 heteroatoms independently selected from nitrogen, oxygen, or sulfur, or:
R⁶ and R⁷ or R⁶ and R^{7′} are optionally taken together to form an optionally substituted 3-8 membered saturated or partially unsaturated ring having 0-4 heteroatoms selected from nitrogen, oxygen, or sulfur;
p is 0-4;
each R⁹ is independently selected from halogen, R, OR, SR, or N(R)₂, or:
two R⁹ on the same carbon are optionally taken together to form an optionally substituted 3-8 membered or partially unsaturated spirofused ring having 0-4 heteroatoms independently selected from nitrogen, oxygen, or sulfur, or:
two R⁹ on the same carbon atom are optionally taken together to form an oxo moiety, an oxime, an optionally substituted hydrazone, an optionally substituted imine, or an optionally substituted C₂₋₆ alkylidene;
Q is a valence bond or an optionally substituted C₁₋₁₀ alkylene chain wherein one, two, or three methylene units of Q are optionally and independently replaced by —O—, —N(R)—, —S—, —C(O)—, —OC(O)—, —C(O)O—, —OC(O)O—, —S(O)—, or —S(O)₂—, —OSO₂O—, —N(R)C(O)—, —C(O)N(R)—, —N(R)C(O)O—, —OC(O)NR—, —N(R)C(O)NR—, or —Cy-, wherein:
each —Cy- is independently a bivalent optionally substituted saturated, partially unsaturated, or aromatic monocyclic or bicyclic ring selected from a 6-10 membered arylene, a 5-10 membered heteroarylene having 1-4 heteroatoms independently selected from oxygen, nitrogen, or sulfur, a 3-8 membered carbocyclylene, or a 3-10 membered heterocyclylene having 1-4 heteroatoms independently selected from oxygen, nitrogen, or sulfur;
R¹⁰ is hydrogen, halogen, an optionally substituted C₁₋₁₀ aliphatic, a suitably protected hydroxyl group, a suitably protected thiol group, a suitably protected amino group, an optionally substituted 3-8 membered saturated, partially unsaturated, or aryl monocyclic ring having 0-4 heteroatoms independently selected from nitrogen, oxygen, or sulfur, an optionally substituted 8-10 membered saturated, partially unsaturated, or aryl bicyclic ring having 0-4 heteroatoms independently selected from nitrogen, oxygen, or sulfur, a detectable moiety, a polymer residue, a peptide, a sugar-containing or sugar-like moiety, or:
wherein when R¹⁰ is a ring, R¹⁰ is optionally substituted at any substitutable carbon with 1-7 R¹¹ and at any substitutable nitrogen with R¹²;
each R¹¹ is independently halogen, R, OR, SR, N(R)₂, N(R)C(O)R, N(R)C(O)OR, N(R)C(O)N(R)₂, N(R)SO₂R, N(R)SO₂OR, S(O)R, SO₂R, OSO₂R, C(O)R, CO₂R, OCO₂R, C(O)N(R)₂, or OC(O)N(R)₂, or wherein:
two R¹¹ are optionally taken together to form an oxo moiety, an oxime, an optionally substituted hydrazone, an optionally substituted imine, an optionally substituted C₂₋₆ alkylidene, or an optionally substituted 3-8 membered saturated or partially unsaturated fused or spirofused ring having 0-4 heteroatoms independently selected from nitrogen, oxygen, or sulfur; and
each R¹² is independently R, OR, S(O)R, SO₂R, OSO₂R, C(O)R, CO₂R, OCO₂R, C(O)N(R)₂, or OC(O)N(R)₂, an optionally substituted aliphatic group, a suitably protected amino group, an optionally substituted 3-8 membered saturated, partially unsaturated, or aryl monocyclic ring having 0-4 heteroatoms independently selected from nitrogen, oxygen, or sulfur, an optionally substituted 8-10 membered saturated, partially unsaturated, or aryl bicyclic ring having 0-4 heteroatoms independently selected from nitrogen, oxygen, or sulfur, or wherein:
R¹² and R¹¹ are optionally taken together to form an optionally substituted 3-8 membered saturated or partially unsaturated fused ring having 0-4 heteroatoms independently selected from nitrogen, oxygen, or sulfur.

2. The compound of claim 1, wherein Q is an optionally substituted C₁₋₁₀ alkylene chain wherein one, two, or three methylene units are independently replaced by —O—, —N(R)—, —S—, —C(O)—, —SO₂—, or —Cy-.

3. The compound of claim 2, wherein Q is —O—.

4. The compound of claim 1, wherein Q is an optionally substituted C₂₋₁₀ alkylene chain wherein two or three methylene units are independently replaced by —O— and —Cy-.

5. The compound of claim 1, wherein Q is a C₂ alkylene chain wherein one methylene unit is replaced by —O— and one methylene unit is replaced by —Cy-.

6. The compound of claim 1, wherein:
Q is an optionally substituted C₂₋₁₀ alkylene chain wherein two or three methylene units are independently replaced by —O— and —Cy-; and
each —Cy- is independently an optionally substituted 3-10 membered heterocyclylene having 1-4 heteroatoms independently selected from oxygen, nitrogen, or sulfur.

7. The compound of claim 6, wherein each —Cy- is independently an optionally substituted 5-7 membered heterocyclylene having 1-3 heteroatoms independently selected from oxygen, nitrogen, or sulfur.

8. The compound of claim 7, wherein —Cy- is selected from tetrahydropyranylene, tetrahydrofuranylene, morpholinylene, thiomorpholinylene, piperidinylene, piperazinylene, pyrrolidinylene, tetrahydrothiophenylene, and tetrahydrothiopyranylene, wherein each ring is optionally substituted.

9. The compound of claim 8, wherein —Cy- is optionally substituted morpholinylene.

10. The compound of claim 1, wherein R¹⁰ is an optionally substituted 3-8 membered heterocycle containing 1-2 heteroatoms selected from nitrogen, oxygen, or sulfur.

11. The compound of claim 1, wherein -Q-R¹⁰ is of the following formula:

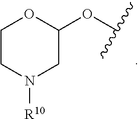

12. The compound of claim 11, wherein R¹⁰ a detectable moiety comprising biotin.

13. The compound of claim 11, wherein R¹⁰ is a protecting group selected from t-butyloxycarbonyl (BOC), ethyloxycarbonyl, methyloxycarbonyl, trichloroethyloxycarbonyl, allyloxycarbonyl (Alloc), benzyloxocarbonyl (CBZ), allyl, phthalimide, benzyl (Bn), fluorenylmethylcarbonyl (Fmoc), formyl, acetyl, chloroacetyl, dichloroacetyl, trichloroacetyl, phenylacetyl, trifluoroacetyl, benzoyl, mesyl, tosyl, and triflyl.

14. The compound according to claim 1, wherein said compound is of formula V-a-xi: 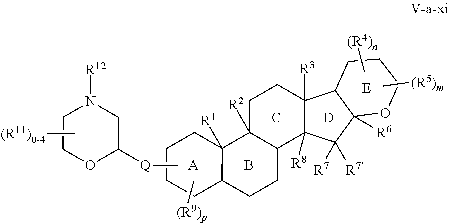
or a pharmaceutically acceptable salt thereof.

15. The compound of claim 1, wherein Q is an optionally substituted C₂₋₁₀ alkylene chain wherein one or two methylene units are independently replaced by —OC(O)NR— or —Cy-.

16. The compound of claim 15, wherein Q is an optionally substituted C₂₋₁₀ alkylene chain wherein two methylene units are independently replaced by —OC(O)NR— and —Cy-.

17. The compound of claim 16, wherein —Cy- is independently an optionally substituted 3-10 membered heterocyclylene having 1-4 heteroatoms independently selected from oxygen, nitrogen, or sulfur.

18. The compound of claim 17, wherein —Cy- is independently an optionally substituted 3-4 membered heterocyclylene having 1-4 heteroatoms independently selected from oxygen, nitrogen, or sulfur.

19. The compound of claim 18, wherein —Cy- is independently an optionally substituted 3-8 membered carbocyclylene.

20. The compound of claim 19, wherein —Cy- is independently an optionally substituted 4 membered carbocyclylene.

21-46. (canceled)
